(19) United States
(12) Patent Application Publication
(10) Pub. No.: US 2026/0096342 A1
(43) Pub. Date: Apr. 2, 2026

(54) HETEROCYCLIC COMPOUND, COMPOSITION FOR ORGANIC LAYER INCLUDING THE SAME AND LIGHT EMITTING DEVICE INCLUDING THE SAME

(71) Applicant: LT MATERIALS CO., LTD., Gyeonggi-do (KR)

(72) Inventors: SEUNG WOO LEE, Gyeonggi-do (KR); JUN TAE MO, Gyeonggi-do (KR); DONG JUN KIM, Gyeonggi-do (KR); DAE HYUK CHOI, Gyeonggi-do (KR)

(21) Appl. No.: 19/339,720

(22) Filed: Sep. 25, 2025

(30) Foreign Application Priority Data
Sep. 27, 2024 (KR) 10-2024-0132056

(51) Int. Cl. H10K 85/60; C07B 59/00; C07D 405/04; C07D 405/14; C07D 409/14; C09K 11/02; H10K 50/12

(52) U.S. Cl. CPC H10K 85/636; C07B 59/002; C07D 405/04; C07D 405/14; C07D 409/14; C09K 11/02; H10K 85/626; H10K 85/633; H10K 85/654; H10K 50/12; H10K 85/615; H10K 85/6572; H10K 85/6574; H10K 85/6576

(57) ABSTRACT

A heterocyclic compound is represented by Formula 1. A light-emitting device includes a first electrode, a second electrode, and an organic layer between the first electrode and the second electrode and including the heterocyclic compound. The organic layer may include an emission layer including a dopant material and a host material including the heterocyclic compound.

### CROSS-REFERENCE TO RELATED APPLICATION AND CLAIM OF PRIORITY

This application claims the benefit under 35 USC § 119 of Korean Patent Application No. 10-2024-0132056, filed on Sep. 27, 2024, in the Korean Intellectual Property Office, the entire disclosure of which is incorporated herein by reference for all purposes.

### BACKGROUND

### 1. Field

The present disclosure relates to a heterocyclic compound and a light-emitting device including the same.

### 2. Description of the Related Art

An organic light-emitting display (OLED) device includes organic light-emitting devices having self-luminescent properties. Since the organic light-emitting display device does not require a separate light source, it may provide a wide viewing angle and a fast response speed, and may enhance contrast and brightness.

In the organic light-emitting device, an organic emission layer is formed for each pixel, and the organic emission layer may be interposed between opposing electrodes. Holes and electrons injected from the respective electrodes recombine in the organic emission layer to generate excitons, and light may be generated through the energy release of the excitons.

Research is being conducted on materials applicable to the organic emission layer to achieve high efficiency and long life organic light-emitting devices.

### SUMMARY

An object of the present disclosure is to provide a heterocyclic compound.

Another object of the present disclosure is to provide a light-emitting device including the heterocyclic compound.

A heterocyclic compound according to the present disclosure may be represented by Chemical Formula 1 below.

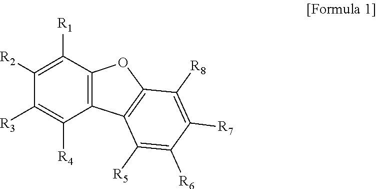

In Chemical Formula 1, two adjacent groups of R₁ to R₄ are linked to form an aromatic ring represented by

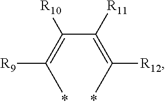

and
among the remaining two groups of R₁ to R₄ that do not form the aromatic ring and R₅ to R₁₂, one is a group represented by Chemical Formula 2 below,
another is a benzocarbazole group having a substituted or unsubstituted aryl group having 6 to 60 carbon atoms or a substituted or unsubstituted heteroaryl group having 2 to 60 carbon atoms bonded to a nitrogen atom, and
the remaining ones are each independently selected from the groups consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; or —P(═O)RR′, wherein two or more adjacent groups among the remaining onesmay optionally be linked to form a substituted or unsubstituted 5- to 9-membered saturated or unsaturated ring, and
R, R′ and R″ are each independently hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

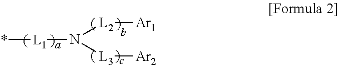

In Chemical Formula 2, Ar₁ and Ar₂ are each independently a substituted or unsubstituted aryl group having 6 to 60 carbon atoms or a substituted or unsubstituted heteroaryl group having 2 to 60 carbon atoms,
L₁, L₂ and L₃ are each independently a direct linkage, a substituted or unsubstituted arylene group having 6 to 60 carbon atoms, or a substituted or unsubstituted heteroarylene group having 2 to 60 carbon atoms,
a, b and c are each independently an integer of 0 to 5,
when a is an integer of 2 to 5, a plurality of L₁ groups are the same or different,
when b is an integer of 2 to 5, a plurality of L₂ groups are the same or different,
when c is an integer of 2 to 5, a plurality of L₃ groups are the same or different, and
* represents a bonding site.

A light-emitting device according to the present disclosure may include: a first electrode; a second electrode disposed on the first electrode; and one or more organic layers interposed between the first electrode and the second electrode. One or more of the organic layers may include the heterocyclic compound.

A composition for an organic layer of an organic light-emitting device according to the present disclosure may include: the heterocyclic compound represented by Chemical Formula 1; and a compound represented by Chemical Formula 4 below.

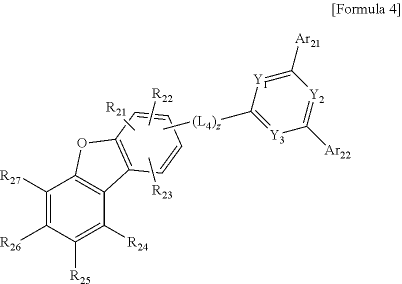

In Chemical Formula 4, any one of R₂₁ to R₂₇ is a substituted or unsubstituted aryl group having 6 to 60 carbon atoms, or a substituted or unsubstituted heteroaryl group having 2 to 60 carbon atoms,
the remaining groups of R₂₁ to R₂₇ are each independently selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a phosphine oxide group; and a silyl group; or two or more adjacent groups are linked to form a substituted or unsubstituted aromatic hydrocarbon ring having 6 to 60 carbon atoms or a substituted or unsubstituted heterocycle having 2 to 60 carbon atoms,
L₄ is a direct linkage, a substituted or unsubstituted arylene group having 6 to 60 carbon atoms, or a substituted or unsubstituted heteroarylene group having 2 to 60 carbon atoms;
z is an integer of 0 to 5;
when z is an integer of 2 to 5, a plurality of L₄ groups are the same or different;
Y₁ to Y₃ are each independently CH, nitrogen (N), oxygen (O), or sulfur(S); and at least two of Y₁ to Y₃ are nitrogen; and
Ar₂₁ and Ar₂₂ are each independently a substituted or unsubstituted aryl group having 6 to 60 carbon atoms, or a substituted or unsubstituted heteroaryl group having 2 to 60 carbon atoms.

According to exemplary embodiments, the light-emitting device may include a heterocyclic compound of one embodiment. The above-described heterocyclic compound may function, for example, as a host material to control the energy band gap and energy levels of the emission layer. Therefore, the light-emitting efficiency and lifetime characteristics of the light-emitting device may be improved by the above-described heterocyclic compound.

### BRIEF DESCRIPTION OF THE DRAWINGS

The above and other objects, features and other advantages of the present disclosure will be more clearly understood from the following detailed description taken in conjunction with the accompanying drawings, in which:

FIGS. 1 to 3 are schematic cross-sectional views of light-emitting devices according to embodiments of the present disclosure, respectively.

### DETAILED DESCRIPTION

Hereinafter, exemplary embodiments of the present disclosure will be described in detail with reference to the accompanying drawings. However, these embodiments are merely illustrative, and the present disclosure is not limited to the specific embodiments described as examples.

As used herein, when a portion is described to “include” a component, unless otherwise specified, it means that the portion does not exclude other components and may further include other components.

As used herein, the * symbol in a chemical formula represents a bonding site.

As used herein, “substitution” means that any hydrogen atom bonded to a carbon atom in a compound is replaced with another substituent. The position of substitution is not limited as long as the substituent is at a substitutable position. If two or more substitutions are made, the two or more substituents may be the same or different.

As used herein, “substituted or unsubstituted” means that at least one hydrogen atom of a compound is unsubstituted or substituted with one or more substituents selected from the following substituents, or is substituted with a substituent in which two or more of the following substituents are linked: deuterium; a halogen group; —CN; an alkyl group having 1 to 60 carbon atoms; an alkenyl group having 2 to 60 carbon atoms; an alkynyl group having 2 to 60 carbon atoms; a haloalkyl group having 1 to 60 carbon atoms; an alkoxy group having 1 to 60 carbon atoms; an aryloxy group having 6 to 60 carbon atoms; an alkylthio group having 1 to 60 carbon atoms; an arylthio group having 6 to 60 carbon atoms; an alkylsulfinyl group having 1 to 60 carbon atoms; an arylsulfinyl group having 6 to 60 carbon atoms; a cycloalkyl group having 3 to 60 carbon atoms; a heterocycloalkyl group having 2 to 60 carbon atoms; an aryl group having 6 to 60 carbon atoms; a heteroaryl group having 2 to 60 carbon atoms; —SiRR′R″; —P(═O)RR′; and —NRR′. R, R′ and R″ are each independently a substituent including at least one of hydrogen; deuterium; a halogen group; an alkyl group; an alkenyl group; an alkoxy group; a cycloalkyl group; a heterocycloalkyl group; an aryl group; and a heteroaryl group.

As used herein, when a substituent is not indicated in a chemical formula or compound structure, it means that a hydrogen atom is bonded to the carbon atom. However, since deuterium (2H) is an isotope of hydrogen, some hydrogen atoms may be deuterium.

As used herein, when a substituent is not indicated in a chemical formula or compound structure, it may mean that all positions that can be substituted are hydrogen or deuterium. In other words, deuterium is an isotope of hydrogen, and some of the hydrogen atoms may be deuterium atoms, in which case the deuterium content may be 0% to 100%.

When a substituent is not indicated in a chemical formula or compound structure, and the deuterium content is 0%, the hydrogen content is 100%, and all substituents are hydrogen, hydrogen and deuterium may nevertheless be present together in the compound unless deuterium is explicitly excluded.

Deuterium is an isotope of hydrogen, having a nucleus composed of one proton and one neutron (a deuteron). It may be represented as hydrogen-2 and its elemental symbol may be written as D or ²H.

Isotopes are atoms with the same atomic number (Z) but different mass numbers (A). Isotopes may also be defined as atoms with the same number of protons but different numbers of neutrons.

As used herein, the content T % of a specific substituent is defined as T2/T1×100=T (%), where the total number of substituents that a basic compound may have is defined as T1, and the number of the specific substituent among them is defined as T2.

In one example, a 20% deuterium content in a phenyl group represented by

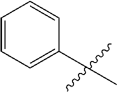 [SMILES: *c1ccccc1]

means that the total number of substituents that the phenyl group may have is 5 (T1 in the formula), and among them, the number of deuterium atoms is 1 (T2 in the formula), which may be represented as 20%. That is, cases where the deuterium content in the phenyl group is 20% may be represented by the structural formula below.

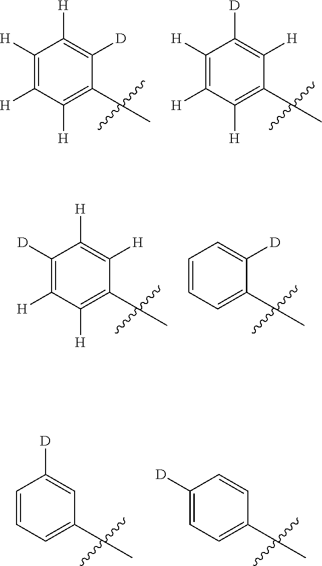 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, “a phenyl group having 0% deuterium content” may refer to a phenyl group that includes no deuterium atoms and has five hydrogen atoms.

As used herein, halogen may be fluorine, chlorine, bromine, or iodine.

As used herein, an alkyl group includes a straight or branched chain having 1 to 60 carbon atoms, and may be further substituted with one or more substituents. The alkyl group may have 1 to 60, 1 to 40, or 1 to 20 carbon atoms. Examples thereof may include a methyl group, an ethyl group, a propyl group, an n-propyl group, an isopropyl group, a butyl group, an n-butyl group, an isobutyl group, a tert-butyl group, a sec-butyl group, a 1-methylbutyl group, a 1-ethylbutyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethylpropyl group, a 1,1-dimethylpropyl group, an isohexyl group, a 2-methylpentyl group, a 4-methylhexyl group, and a 5-methylhexyl group, but are not limited thereto.

As used herein, the alkenyl group includes a straight or branched chain having 2 to 60 carbon atoms, and may be further substituted with one or more substituents. The alkenyl group may have 2 to 60, 2 to 40, or 2 to 20 carbon atoms. Examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl group, a 2-phenylvinyl group, a 2,2-diphenylvinyl group, a 2-phenyl-2-(naphthyl-1-yl) vinyl group, a 2,2-bis(diphenyl) vinyl group, a stilbenyl group, and a styrenyl group, but are not limited thereto.

As used herein, an alkynyl group includes a straight or branched chain having 2 to 60 carbon atoms, and may be further substituted with one or more substituents. The alkynyl group may have 2 to 60, 2 to 40, or 2 to 20 carbon atoms.

As used herein, a haloalkyl group refers to an alkyl group substituted with a halogen group. Examples thereof may include —CF₃ and —CF₂CF₃, but are not limited thereto.

As used herein, a cycloalkyl group includes a monocyclic or polycyclic group having 3 to 60 carbon atoms, and may be further substituted with one or more substituents. In this context, a polycyclic group refers to a group in which a cycloalkyl group is directly linked to or condensed with another cyclic group. The other cyclic group may be a cycloalkyl group, but may also be another type of cyclic group, such as a heterocycloalkyl group, an aryl group, or a heteroaryl group. The number of carbon atoms in the cycloalkyl group may be 3 to 60, 3 to 40, or 5 to 20. Examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, and a cyclooctyl group, but are not limited thereto.

As used herein, a heterocycloalkyl group includes a monocyclic or polycyclic group having 2 to 60 carbon atoms and including at least one heteroatom selected from O, S, Se, N and Si, and may be further substituted with one or more substituents. In this context, the term “polycyclic” refers to a group in which a heterocycloalkyl group is directly linked to or condensed with another ring group. The other ring group may be a heterocycloalkyl group, but may also be another type of ring group, such as a cycloalkyl group, an aryl group, or a heteroaryl group. The heterocycloalkyl group may have 2 to 60, 2 to 40, or 3 to 20 carbon atoms.

As used herein, an aryl group includes a monocyclic or polycyclic group having 6 to 60 carbon atoms, and may be further substituted with one or more substituents. In this context, the term “polycyclic” refers to a group in which an aryl group is directly linked to or condensed with another ring group. The other ring group may be an aryl group, but may also be another type of ring group, such as a cycloalkyl group, a heterocycloalkyl group, or a heteroaryl group. The aryl group may include a spiro ring structure. The aryl group may have 6 to 60, 6 to 40, or 6 to 25 carbon atoms. Specific examples of the aryl group may include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, and condensed ring groups thereof, but are not limited thereto.

As used herein, the terphenyl group may be selected from the following structures.

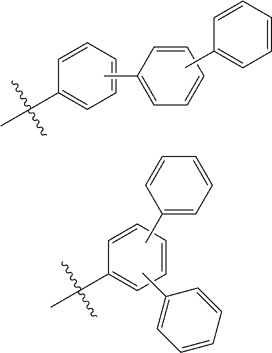

As used herein, the fluorenyl group may be substituted, and adjacent substituents may be bonded to form a ring.

The substituted fluorenyl group may be represented by the following structural formula, but is not limited thereto.

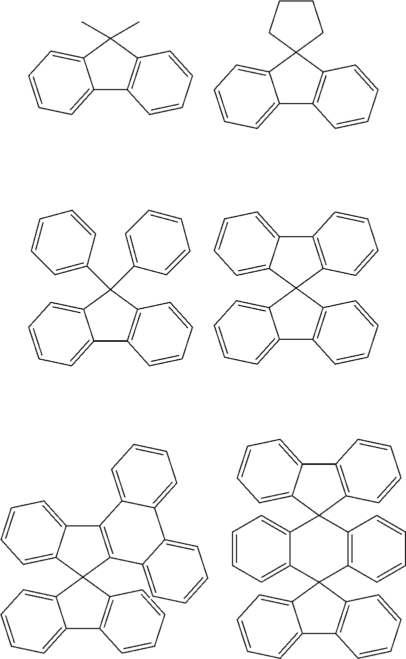

As used herein, an alkoxy group is represented by —O(R101), and R101 may be selected from the examples of the alkyl group described above.

As used herein, an aryloxy group is represented by —O(R102), and R102 may be selected from the examples of the aryl group described above.

As used herein, an alkylthio group is represented by —S(R103), and R103 may be selected from the examples of the alkyl group described above.

As used herein, an arylthio group is represented by —S(R104), and R104 may be selected from the examples of the aryl group described above.

As used herein, an alkylsulfinyl group is represented by —S(═O)₂(R105), and R105 may be selected from the examples of the alkyl group described above.

As used herein, an arylsulfinyl group is represented by —S(═O)₂(R106), and R106 may be selected from the examples of the aryl group described above.

As used herein, a heteroaryl group includes a monocyclic or polycyclic group having 2 to 60 carbon atoms and including at least one heteroatom selected from S, O, Se, N, and Si, and may be further substituted with one or more substituents. In this context, the term “polycyclic” refers to a group in which a heteroaryl group is directly linked to or condensed with another ring group. The other ring group may be a heteroaryl group, but may also be another type of ring group, such as a cycloalkyl group, a heterocycloalkyl group, or an aryl group. The heteroaryl group may have 2 to 60, 2 to 40, or 3 to 25 carbon atoms. Examples of the heteroaryl group may include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, a spirobi (dibenzosilole) group, a dihydrophenazine group, a phenoxazine group, a phenanthridine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiathiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azacillin group, a pyrazolo[1,5-c]quinazoline group, a pyrido[1,2-b]indazole group, a pyrido[1,2-a]imidazo[1,2-e]indolin group, and a 5,11-dihydroindeno[1,2-b]carbazole group, but are not limited thereto.

As used herein, when a substituent is a carbazole group, it means that the substituent is bonded to the nitrogen or carbon of the carbazole group.

As used herein, when a carbazole group is substituted, an additional substituent may be present on the nitrogen or carbon of the carbazole group.

As used herein, examples of a benzocarbazole group may include any one of the following structures.

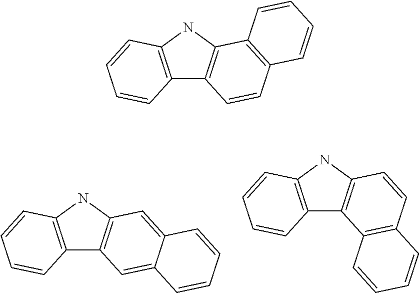

As used herein, examples of a dibenzocarbazole group may include any one of the following structures.

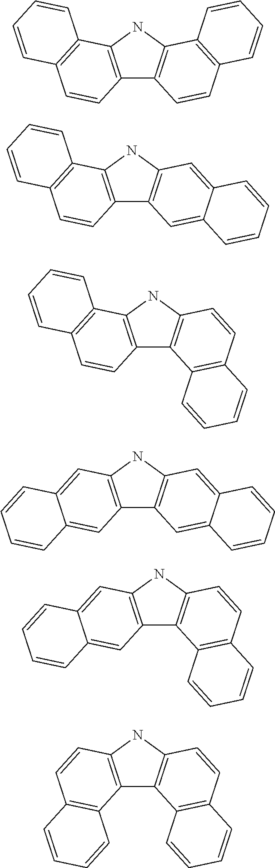

As used herein, examples of a naphthobenzofuran group may include any one of the following structures.

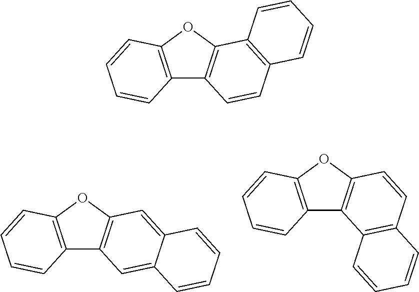

As used herein, examples of a naphthobenzothiophene group may include any one of the following structures.

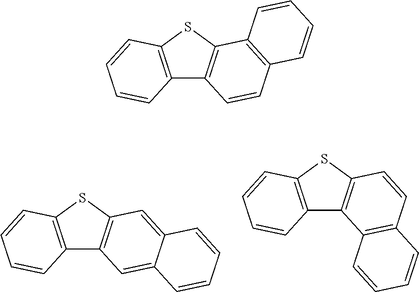

Among the substituents, —SiRR′R″ is a silyl group, which is a substituent that includes Si, and the Si atom is directly bonded as a radical. R, R′ and R″ may be the same or different, and each independently may be a substituent including at least one of hydrogen; deuterium; a halogen group; an alkyl group; an alkenyl group; an alkoxy group; a cycloalkyl group; a heterocycloalkyl group; an aryl group; and a heteroaryl group. Examples of the silyl group may include,

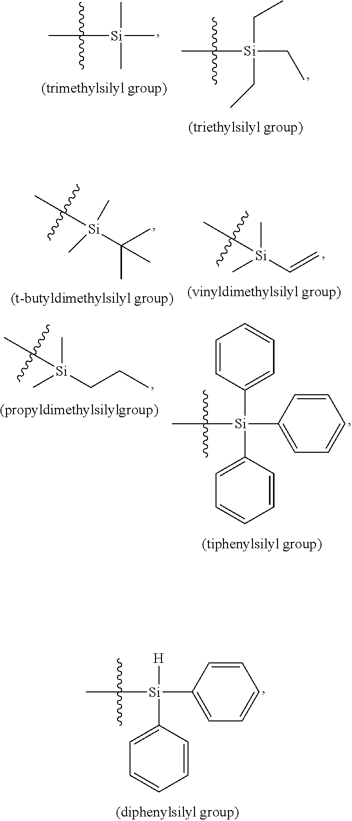

and

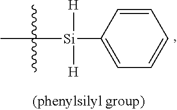 [SMILES: *[SiH2]c1ccccc1]

but are not limited thereto.

Among the substituents, —P(═O)RR′ is a phosphine oxide group, wherein R and R′ may be the same or different, and each independently may be a substituent including at least one of hydrogen; deuterium; a halogen group; an alkyl group; an alkenyl group; an alkoxy group; a cycloalkyl group; a heterocycloalkyl group; an aryl group; and a heteroaryl group, and in particular may be an alkyl group or an aryl group. The alkyl group and aryl group may be selected from the examples described above. For example, the phosphine oxide group may include a dimethylphosphine oxide group, a diphenylphosphine oxide group, and a dinaphthylphosphine oxide group, but is not limited thereto.

Among the substituents, —NRR′ is an amine group, wherein R and R′ may be the same or different, and each independently may be a substituent including at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂, a monoalkylamine group, a monoarylamine group, a monoheteroarylamine group, a dialkylamine group, a diarylamine group, a diheteroarylamine group, an alkylarylamine group, an alkylheteroarylamine group, and an arylheteroarylamine group, and the number of carbon atoms is not particularly limited, but is preferably 1 to 30. Examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methylanthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, and a biphenyltriphenylenylamine group, but are not limited thereto.

As used herein, an arylene group may be selected from the examples of the aryl group described above, except that it is divalent.

As used herein, a heteroarylene group may be selected from the examples of the heteroaryl group described above, except that it is divalent.

As used herein, the term “adjacent” group may refer to a substituent that is substituted on an atom directly linked to the atom substituted by the substituent, a substituent that is sterically closest to the substituent, or another substituent substituted on the atom substituted by the substituent. For example, two substituents substituted at the ortho positions of a benzene ring and two substituents substituted on the same carbon atom of an aliphatic ring may be interpreted as “adjacent” groups.

Hydrocarbon rings and heterocycles that can be formed by adjacent groups may include aliphatic hydrocarbon rings, aromatic hydrocarbon rings, aliphatic heterocycles, and aromatic heterocycles, and the structures exemplified by the above-described cycloalkyl groups, aryl groups, heterocycloalkyl groups, and heteroaryl groups may apply thereto, except that they are not monovalent.

Generally, compounds bonded to hydrogen and compounds substituted with deuterium exhibit different thermodynamic behaviors. The reason is that the mass of the deuterium atom is twice that of hydrogen. Due to this difference in atomic mass, deuterium has the characteristic of having lower vibrational energy.

In addition, the single bond dissociation energy (BDE) of carbon-deuterium is higher than that of carbon-hydrogen. Therefore, structures substituted with deuterium have increased thermal stability, resulting in improved lifetime of devices utilizing them.

When a compound is deposited on a silicon wafer, substances including deuterium tend to pack more tightly at intermolecular distance. In addition, observation of the thin film surface with an atomic force microscope (AFM) reveals that thin films formed from deuterium-containing compounds are deposited with a more uniform surface and without aggregation.

The heterocyclic compound of Formula 1 of the present disclosure has a deuterium substitution ratio of greater than 0% and not more than 100%. When deuterium is substituted, the ground state energy is lowered compared to hydrogen-substituted compounds. Furthermore, as the carbon-deuterium bond length decreases, the molecular hardcore volume decreases. This can reduce electrical polarizability and weaken intermolecular interactions, resulting in a more stable stacking structure during device fabrication.

These characteristics create an amorphous state in the thin film, thereby lowering the crystallinity. In other words, the heterocyclic compound of Chemical Formula 1 may be effective in improving the heat resistance of OLED devices, thereby enhancing their lifetime and operating characteristics.

The heterocyclic compound according to the present disclosure may be represented by Chemical Formula 1 below.

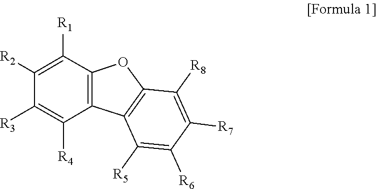

In Chemical Formula 1, two adjacent groups of R₁ to R₄ are linked to form an aromatic ring represented by

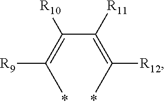

and
among the remaining two groups of R₁ to R₄ that do not form the aromatic ring and R₅ to R₁₂,
one may be a group represented by Chemical Formula 2 below, another may be a benzocarbazole group having a substituted or unsubstituted aryl group having 6 to 60 carbon atoms or a substituted or unsubstituted heteroaryl group having 2 to 60 carbon atoms bonded to a nitrogen atom, and the remaining ones may be each independently selected from the groups consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; or —P(═O)RR′, wherein two or more adjacent groups among the remaining ones may optionally be linked to form a substituted or unsubstituted 5- to 9-membered saturated or unsaturated ring, and R, R′ and R″ are each independently hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

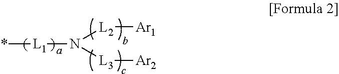

In Chemical Formula 2, Ar₁ and Ar₂ may each independently be a substituted or unsubstituted aryl group having 6 to 60 carbon atoms or a substituted or unsubstituted heteroaryl group having 2 to 60 carbon atoms.

L₁, L₂ and L₃ may each independently be a direct linkage, a substituted or unsubstituted arylene group having 6 to 60 carbon atoms, or a substituted or unsubstituted heteroarylene group having 2 to 60 carbon atoms. For example, L₁, L₂ and L₃ may each independently be a direct linkage or a substituted or unsubstituted arylene group having 6 to 20 carbon atoms.

a, b and c may each independently be 0 or an integer of 1 to 5. For example, a, b and c may each independently be 0, 1 or 2.

When a is an integer of 2 to 5, a plurality of L₁ groups may be the same or different. When bis an integer of 2 to 5, a plurality of L₂ groups may be the same or different. When c is an integer of 2 to 5, a plurality of L₃ groups may be the same or different.

* represents a bonding site.

According to exemplary embodiments, in Chemical Formula 1, R₁ and R₂ may be linked to form an aromatic ring represented by

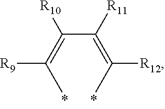

R₂ and R₃ may be linked to form an aromatic ring represented by

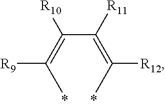

or R₃ and R₄ may be linked to form an aromatic ring represented by

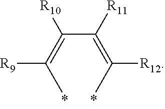

According to exemplary embodiments, the heterocyclic compound may be represented by any one selected from Chemical Formulae 1-1 to 1-3 below.

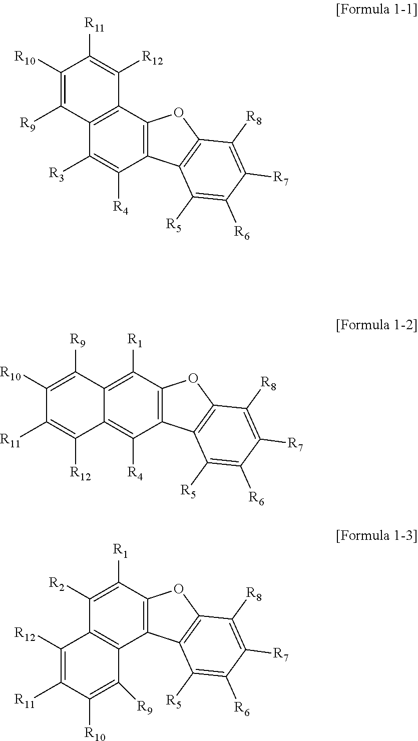

In Chemical Formula 1-1, one of R₃, R₄ and R₅ to R₁₂ may be represented by Chemical Formula 2, the other may be a benzocarbazole group having a substituted or unsubstituted aryl group having 6 to 60 carbon atoms or a substituted or unsubstituted heteroaryl group having 2 to 60 carbon atoms bonded to a nitrogen atom, and the others may be hydrogen or a substituent. The substituents may each independently be selected from deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, and two or more adjacent groups among the substituents may optionally be linked to form a substituted or unsubstituted 5- to 9-membered saturated or unsaturated ring.

In Chemical Formula 1-2, one of R₁, R₄ and R₅ to R₁₂ may be represented by Chemical Formula 2 described below, the other may be a benzocarbazole group having a substituted or unsubstituted aryl group having 6 to 60 carbon atoms or a substituted or unsubstituted heteroaryl group having 2 to 60 carbon atoms bonded to a nitrogen atom, and the others may be hydrogen or substituents, or may be linked to form a ring.

In Chemical Formula 1-3, one of R₁, R₄ and R₅ to R₁₂ may be represented by Chemical Formula 2 described below, the other may be a benzocarbazole group having a substituted or unsubstituted aryl group having 6 to 60 carbon atoms or a substituted or unsubstituted heteroaryl group having 2 to 60 carbon atoms bonded to a nitrogen atom, and the others may be hydrogen or substituents, or may be linked to form a ring.

According to exemplary embodiments, in Chemical Formulae 1-1 to 1-3, R₉ to R₁₂ may each independently be hydrogen or a substituent, or may be linked to form a ring. In some embodiments, two adjacent groups of R₉ to R₁₂, for example, R₉ and R₁₀, R₁₀ and R₁₁, or R₁₁ and R₁₂, may be linked to form a substituted or unsubstituted 5- to 9-membered saturated or unsaturated ring. For example, two adjacent groups of R₉ to R₁₂ may be linked to form a substituted or unsubstituted aromatic ring, a substituted or unsubstituted 5- to 9-membered heterocycle, or a substituted or unsubstituted 5- to 9-membered saturated hydrocarbon ring.

According to exemplary embodiments, in Chemical Formulae 1-1 to 1-3, R₉ to R₁₂ may each independently be hydrogen or deuterium.

According to exemplary embodiments, the heterocyclic compound may be represented by any one of Chemical Formulae 1-4 to 1-6 below.

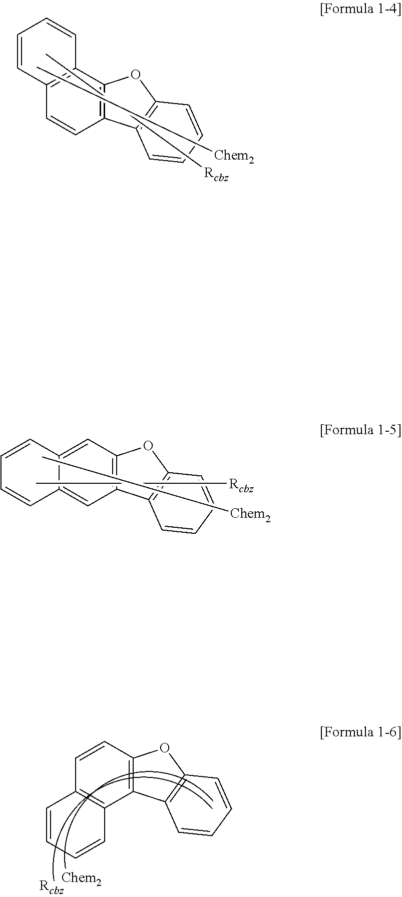

In Chemical Formulae 1-4 to 1-6, R_{cbz} may be a benzocarbazole group having a substituted or unsubstituted aryl group having 6 to 60 carbon atoms or a substituted or unsubstituted heteroaryl group having 2 to 60 carbon atoms bonded to a nitrogen atom, and Chem₂ may be a group represented by Chemical Formula 2 below. Carbon atoms not separately designated as substituted may include hydrogen or deuterium.

In exemplary embodiments, Ar₁ and Ar₂ may each independently be a substituted or unsubstituted aryl group having 6 to 40 carbon atoms or a substituted or unsubstituted heteroaryl group having 2 to 40 carbon atoms. In some embodiments, Ar₁ and Ar₂ may each independently be a substituted or unsubstituted aryl group having 6 to 20 carbon atoms or a substituted or unsubstituted heteroaryl group having 2 to 15 carbon atoms. Ar₁ and Ar₂ may be the same or different.

For example, in Chemical Formula 2, Ar₁ and Ar₂ may each independently be a substituted or unsubstituted phenyl group; a substituted or unsubstituted biphenyl group; a substituted or unsubstituted terphenyl group; a substituted or unsubstituted naphthyl group; a substituted or unsubstituted anthryl group; a substituted or unsubstituted dimethylfluorenyl group; a substituted or unsubstituted diethylfluorenyl group; a substituted or unsubstituted spirobifluorenyl group; a substituted or unsubstituted furan group; a substituted or unsubstituted thiophene group; a substituted or unsubstituted benzofuran group; a substituted or unsubstituted benzothiophene group; a substituted or unsubstituted dibenzofuran group; a substituted or unsubstituted dibenzothiophene group; a substituted or unsubstituted carbazole group; or a combination thereof.

For example, in Chemical Formula 2, Ar₁ and Ar₂ may each independently be a phenyl group substituted or unsubstituted with deuterium; a biphenyl group substituted or unsubstituted with deuterium; a terphenyl group substituted or unsubstituted with deuterium; a naphthyl group substituted or unsubstituted with deuterium; a dimethylfluorenyl group substituted or unsubstituted with deuterium; a diphenylfluorenyl group substituted or unsubstituted with deuterium; a spirobifluorenyl group substituted or unsubstituted with deuterium; a furan group substituted or unsubstituted with deuterium; a thiophene group substituted or unsubstituted with deuterium; a benzofuran group substituted or unsubstituted with deuterium; a benzothiophene group substituted or unsubstituted with deuterium; a dibenzofuran group substituted or unsubstituted with deuterium; a dibenzothiophene group substituted or unsubstituted with deuterium; an anthryl group substituted or unsubstituted with deuterium; a carbazole group substituted or unsubstituted with deuterium; or a combination thereof.

According to some embodiments, in Chemical Formula 2, L₁, L₂ and L₃ may each independently be a direct linkage, a substituted or unsubstituted arylene group having 6 to 40 carbon atoms, or a substituted or unsubstituted heteroarylene group having 2 to 40 carbon atoms. According to some embodiments, in Chemical Formula 2, L₁, L₂ and L₃ may each independently be a direct linkage, a substituted or unsubstituted arylene group having 6 to 20 carbon atoms, or a substituted or unsubstituted heteroarylene group having 2 to 15 carbon atoms. L₁, L₂ and L₃ may be the same or different.

According to some embodiments, in Chemical Formula 2, L₁, L₂ and L₃ may each independently be a direct linkage, a substituted or unsubstituted arylene group having 6 to 10 carbon atoms, for example, a substituted or unsubstituted phenylene group, or a substituted or unsubstituted naphthalene group.

According to exemplary embodiments, in Chemical Formula 2, L₁, L₂ and L₃ may each independently be a direct linkage or a substituted or unsubstituted arylene group having 6 to 20 carbon atoms and optionally substituted with deuterium. For example, L₁, L₂ and L₃ may each independently be a direct linkage or a substituted or unsubstituted phenylene group.

According to some embodiments, in Chemical Formula 2, a, b and c may each independently be 0 or an integer from 1 to 5.

In Chemical Formula 2, when a is an integer from 2 to 5, a plurality of L₁ groups may be the same or different. When b is an integer from 2 to 5, a plurality of L₂ groups may be the same or different. When c is an integer from 2 to 5, a plurality of L₃ groups may be the same or different.

According to some embodiments, in Chemical Formula 2, a, b and c may each independently be 0, 1 or 2.

According to some embodiments, Chemical Formula 2 may be represented by any one of Chemical Formulae 2-1 to 2-8 below.

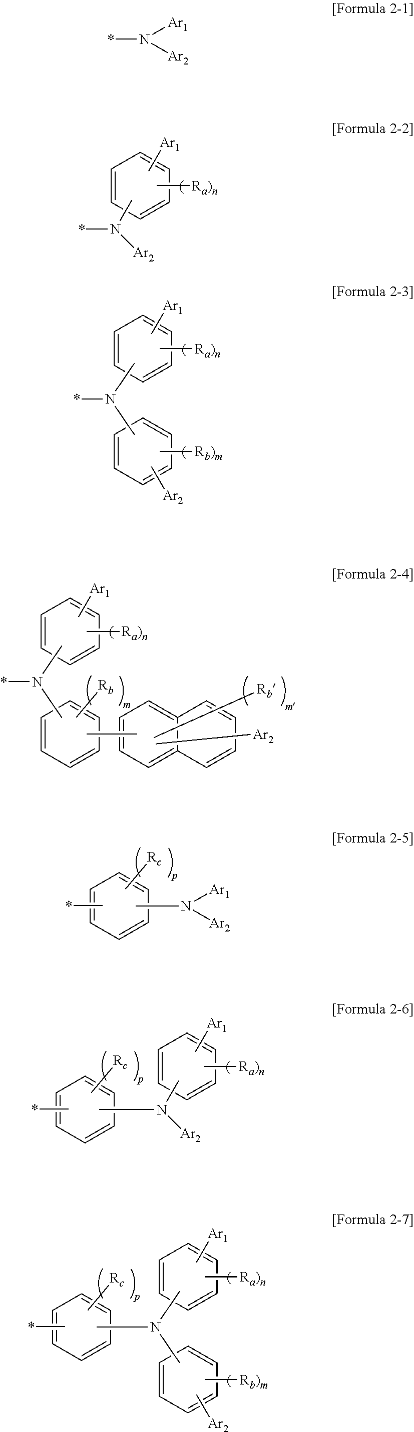

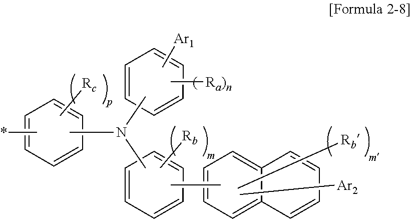

In Chemical Formulae 2-1 to 2-8. Ar₁ and Ar₂ may be the same as defined in Formula 2 above.

Rₐ, R_{b}, R_{b}′ and R_{c} may each independently be hydrogen or deuterium, n, m and p may each independently be 0 or an integer from 1 to 4, and m′ may be an integer of 0 to 6.

When n, m, m′ and p are each independently integers 2 or more, a plurality of Rₐ, R_{b}, R_{b}′ and R_{c} groups may each be the same or different.

In Chemical Formulae 2-1 to 2-8, Rₐ, R_{b}, R_{b}′ and R_{c} may each independently be hydrogen or deuterium, n, m and p may each independently be 0 or an integer from 1 to 4, and m′ may be 0 or an integer of 1 to 6.

In some embodiments, n may be 0, 3 or 4.

In some embodiments, m may be 0, 3 or 4.

In some embodiments, m′ may be 0, 1, 2, 3, 4 or 6.

In some embodiments, p may be 0, 3 or 4.

According to some embodiments, when n, m, m′ and p are each independently integers of 2 or more, a plurality of Rₐ, R_{b}, R_{b}′ and R_{c} groups may each be the same or different.

According to some embodiments, among the remaining two groups of R₁ to R₄ in Chemical Formula 1 that do not form the ring and R₅ to R₁₂, excluding the group represented by Chemical Formula 2, any one may be a benzocarbazole group having a substituted or unsubstituted aryl group having 6 to 60 carbon atoms or a substituted or unsubstituted heteroaryl group having 2 to 60 carbon atoms bonded to a nitrogen atom.

According to some embodiments, the benzocarbazole group may have a substituted or unsubstituted aryl group having 6 to 40 carbon atoms or a substituted or unsubstituted heteroaryl group having 2 to 40 carbon atoms bonded to a nitrogen atom. According to some embodiments, the benzocarbazole group may have a substituted or unsubstituted aryl group having 6 to 20 carbon atoms or a substituted or unsubstituted heteroaryl group having 2 to 15 carbon atoms bonded to a nitrogen atom.

According to some embodiments, the benzocarbazole group may be represented by any one of Chemical Formulae 3-1 to 3-4 below.

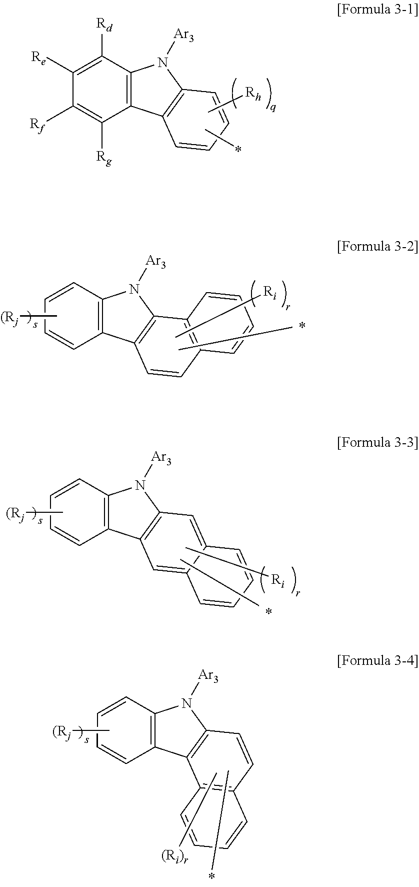

In Chemical Formulae 3-1 to 3-4, Ar₃ may be an aryl group having 6 to 60 carbon atoms or a heteroaryl group having 2 to 60 carbon atoms. In some embodiments, Ar₃ may be an aryl group having 6 to 40 carbon atoms or a heteroaryl group having 2 to 40 carbon atoms. For example, Ar₃ may be a substituted or unsubstituted phenyl group.

In Chemical Formula 3-1, two adjacent groups of R_{d}, Rₑ, R_{f} and R_{g} may be linked to form an aromatic ring represented by

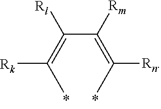

According to some embodiments, in Chemical Formula 3-1, R_{d} and Rₑ may be linked to form an aromatic ring represented by

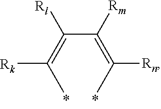

Rₑ and R_{f} may be linked to form an aromatic ring represented by

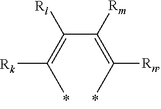

or R_{f} and R_{g} may be linked to form an aromatic ring represented by

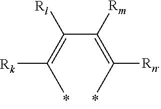

In Chemical Formula 3-1, the remaining two groups of R_{d}, Rₑ, R_{f} and R_{g} that do not form the ring, and Rₕ, Rₖ, Rₗ, Rₘ and Rₙ may each independently be selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein the remaining two groups of R_{d}, Rₑ, R_{f} and R_{g} that do not form the ring, and two or more adjacent groups among Rₕ, Rₖ, Rₗ, Rₘ and Rₙ may optionally be linked to form a substituted or unsubstituted 5- to 9-membered saturated or unsaturated ring.

According to some embodiments, in Chemical Formulae 3-1 to 3-4, Rₖ, Rₗ, Rₘ and Rₙ may each independently be hydrogen or a substituent, or may be linked to form a ring. According to some embodiments, adjacent two groups of Rₖ, Rₗ, Rₘ and Rₙ, for example, Rₖ and Rₗ, Rₗ and Rₘ, or Rₘ and Rₙ, may be linked to form a substituted or unsubstituted 5- to 9-membered saturated or unsaturated ring. For example, adjacent two groups of Rₖ, Rₗ, Rₘ and Rₙ may be linked to form a substituted or unsubstituted aromatic ring, a substituted or unsubstituted 5- to 9-membered heterocycle, or a substituted or unsubstituted 5- to 9-membered saturated hydrocarbon ring.

According to some embodiments, in Chemical Formula 3-1, the remaining two groups of R_{d}, Rₑ, R_{f} and R_{g} that do not form the ring, and Rₕ, Rₖ, Rₗ, Rₘ and Rₙ may each independently be hydrogen or deuterium.

In Chemical Formula 3-1, q is an integer from 0 to 3, and when q is 2 or 3, a plurality of Rₕ groups may be the same or different.

In Chemical Formulae 3-2 to 3-4, Rₕ and Rᵢ may each independently be selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, and two or more adjacent Rₕ and Rᵢ groups may optionally be linked to form a substituted or unsubstituted 5- to 9-membered saturated or unsaturated ring.

R, R′ and R″ may each independently be hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

According to some embodiments, in Chemical Formulae 3-2 to 3-4, Rₕ and Rᵢ may each independently be hydrogen or deuterium.

In Chemical Formulae 3-2 to 3-4, r is an integer from 0 to 5, and when r is an integer from 2 to 5, a plurality of Rᵢ groups may be the same or different. s is an integer from 0 to 4, and when s is an integer from 2 to 4, a plurality of Rⱼ groups may be the same or different.

According to some embodiments, the deuterium content of the compound of Chemical Formula 1 may be 0%, or greater than 0 and not more than 100%.

According to some embodiments, the deuterium content of the compound of Chemical Formula 1 may be 0%, or 5% to 100%.

According to some embodiments, the deuterium content of the compound of Chemical Formula 1 may be 0%, or 10% to 100%.

According to some embodiments, the deuterium content of the compound of Chemical Formula 1 may be 0%, or 15% to 100%.

According to some embodiments, the deuterium content of the compound of Chemical Formula 1 may be 0%, or 20% to 100%.

According to some embodiments, the deuterium content of the compound of Chemical Formula 1 may be 0%, or 25% to 100%.

According to some embodiments, the deuterium content of the compound of Chemical Formula 1 may be 0%, or 30% to 100%.

According to some embodiments, the deuterium content of the compound of Chemical Formula 1 may be 0%, or 50% to 100%.

According to some embodiments, the deuterium content of the compound of Chemical Formula 1 may be 0%, or 70% to 100%.

According to some embodiments, the deuterium content of the compound of Chemical Formula 1 may be 0%, or 90% to 100%.

According to some embodiments, the deuterium content of the compound of Chemical Formula 1 may be 0%.

According to some embodiments, the deuterium content of the compound of Chemical Formula 1 may be 30% to 100%.

According to some embodiments, the deuterium content of the compound of Chemical Formula 1 may be 50% to 100%.

According to some embodiments, the deuterium content of the compound of Chemical Formula 1 may be 70% to 100%.

According to some embodiments, the deuterium content of the compound of Chemical Formula 1 may be 90% to 100%.

The heterocyclic compound may be represented by any one of Chemical Formulae below.

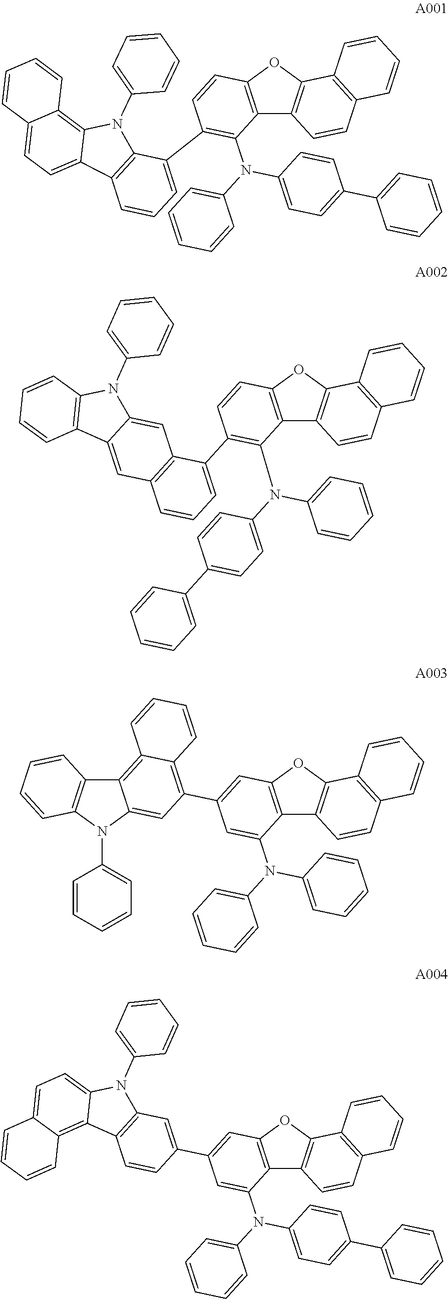 [SMILES: c1ccc(-c2ccc(N(c3ccccc3)c3c(-c4cccc5c6ccc7ccccc7c6n(-c6ccccc6)c45)ccc4oc5c6ccccc6ccc5c34)cc2)cc1 ; c1ccc(-c2ccc(N(c3ccccc3)c3c(-c4cccc5cc6c7ccccc7n(-c7ccccc7)c6cc45)ccc4oc5c6ccccc6ccc5c34)cc2)cc1 ; c1ccc(N(c2ccccc2)c2cc(-c3cc4c(c5ccccc35)c3ccccc3n4-c3ccccc3)cc3oc4c5ccccc5ccc4c23)cc1 ; c1ccc(-c2ccc(N(c3ccccc3)c3cc(-c4ccc5c6c7ccccc7ccc6n(-c6ccccc6)c5c4)cc4oc5c6ccccc6ccc5c34)cc2)cc1]

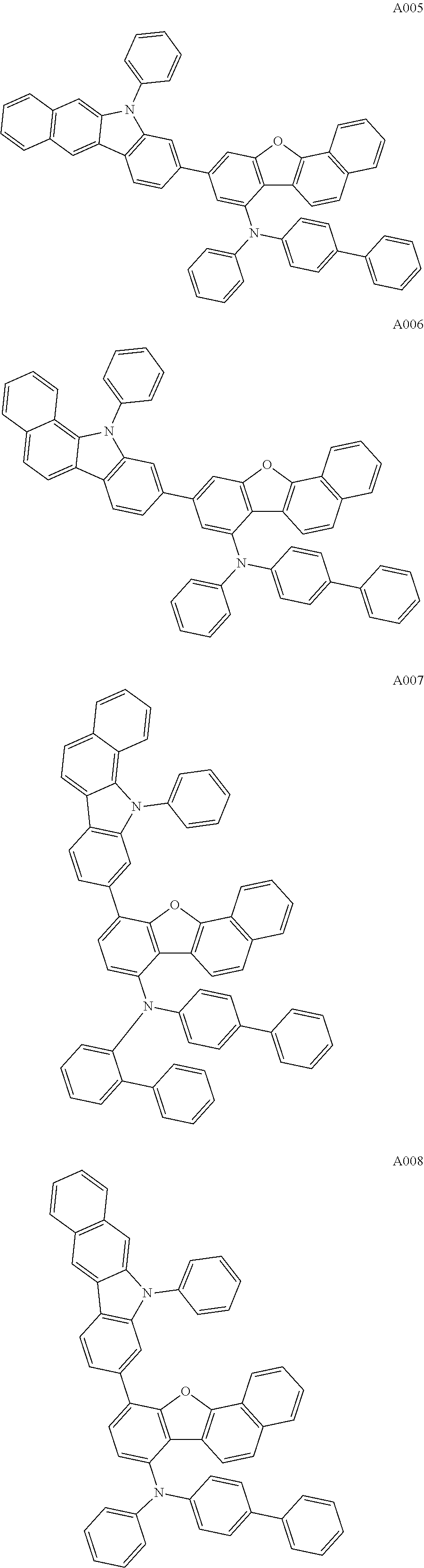 [SMILES: c1ccc(-c2ccc(N(c3ccccc3)c3cc(-c4ccc5c6cc7ccccc7cc6n(-c6ccccc6)c5c4)cc4oc5c6ccccc6ccc5c34)cc2)cc1 ; c1ccc(-c2ccc(N(c3ccccc3)c3cc(-c4ccc5c6ccc7ccccc7c6n(-c6ccccc6)c5c4)cc4oc5c6ccccc6ccc5c34)cc2)cc1 ; c1ccc(-c2ccc(N(c3ccccc3-c3ccccc3)c3ccc(-c4ccc5c6ccc7ccccc7c6n(-c6ccccc6)c5c4)c4oc5c6ccccc6ccc5c34)cc2)cc1 ; c1ccc(-c2ccc(N(c3ccccc3)c3ccc(-c4ccc5c6cc7ccccc7cc6n(-c6ccccc6)c5c4)c4oc5c6ccccc6ccc5c34)cc2)cc1]

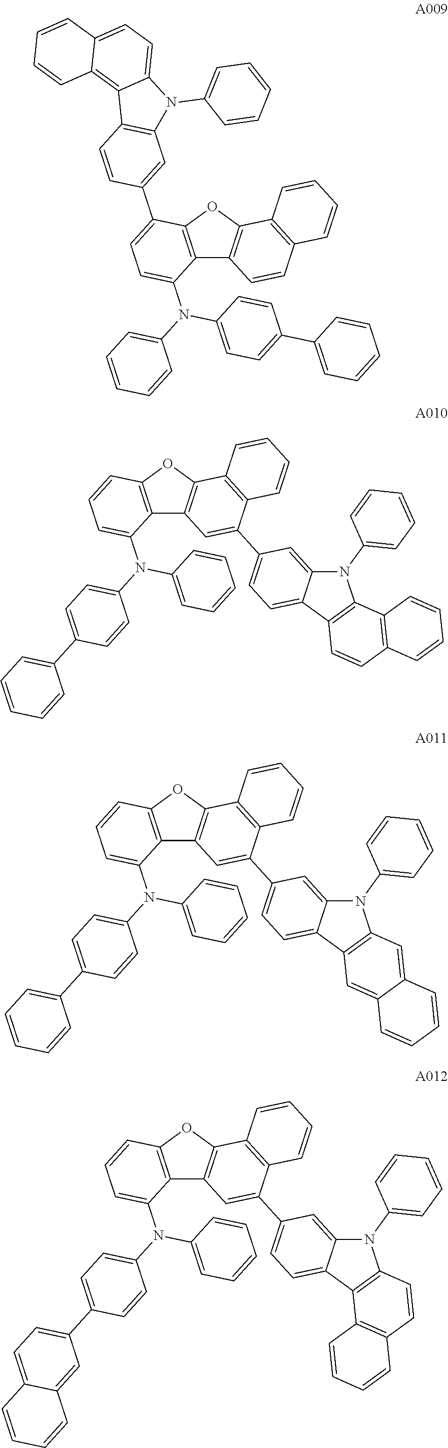 [SMILES: c1ccc(-c2ccc(N(c3ccccc3)c3ccc(-c4ccc5c6c7ccccc7ccc6n(-c6ccccc6)c5c4)c4oc5c6ccccc6ccc5c34)cc2)cc1 ; c1ccc(-c2ccc(N(c3ccccc3)c3cccc4oc5c6ccccc6c(-c6ccc7c8ccc9ccccc9c8n(-c8ccccc8)c7c6)cc5c34)cc2)cc1 ; c1ccc(-c2ccc(N(c3ccccc3)c3cccc4oc5c6ccccc6c(-c6ccc7c8cc9ccccc9cc8n(-c8ccccc8)c7c6)cc5c34)cc2)cc1 ; c1ccc(N(c2ccc(-c3ccc4ccccc4c3)cc2)c2cccc3oc4c5ccccc5c(-c5ccc6c7c8ccccc8ccc7n(-c7ccccc7)c6c5)cc4c23)cc1]

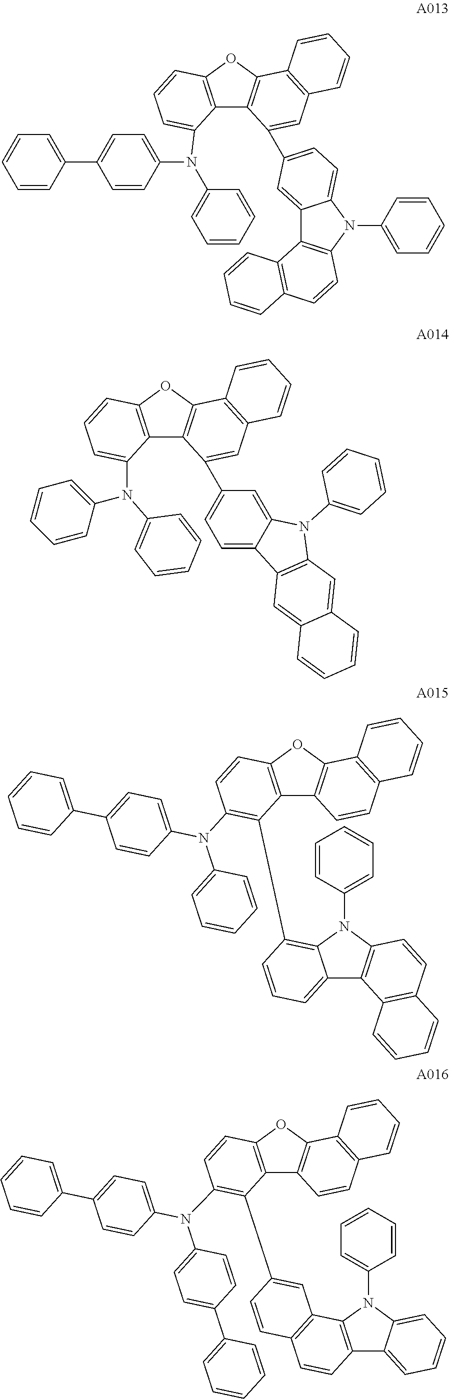

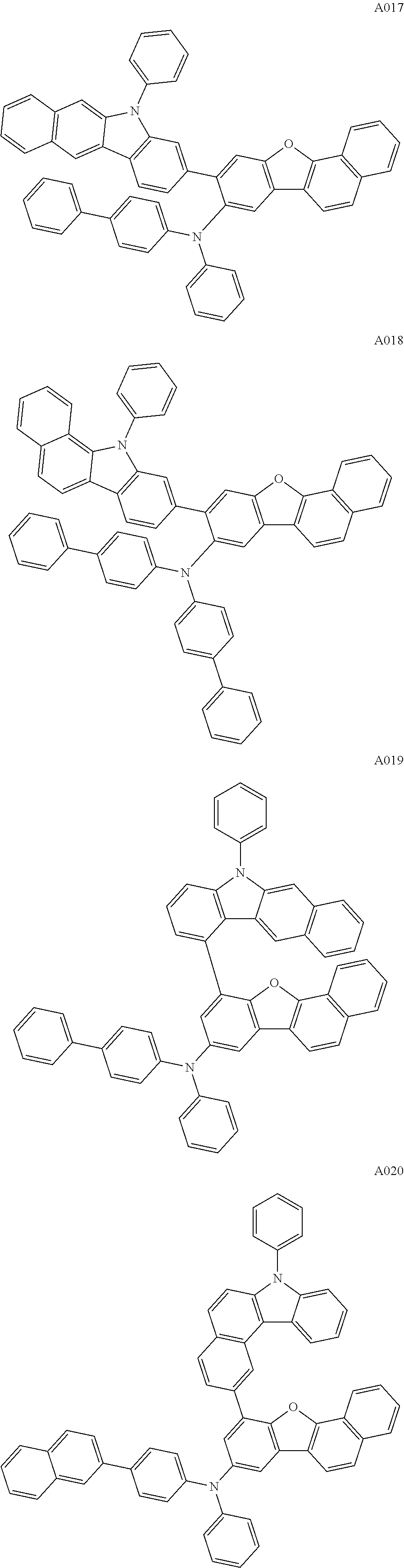

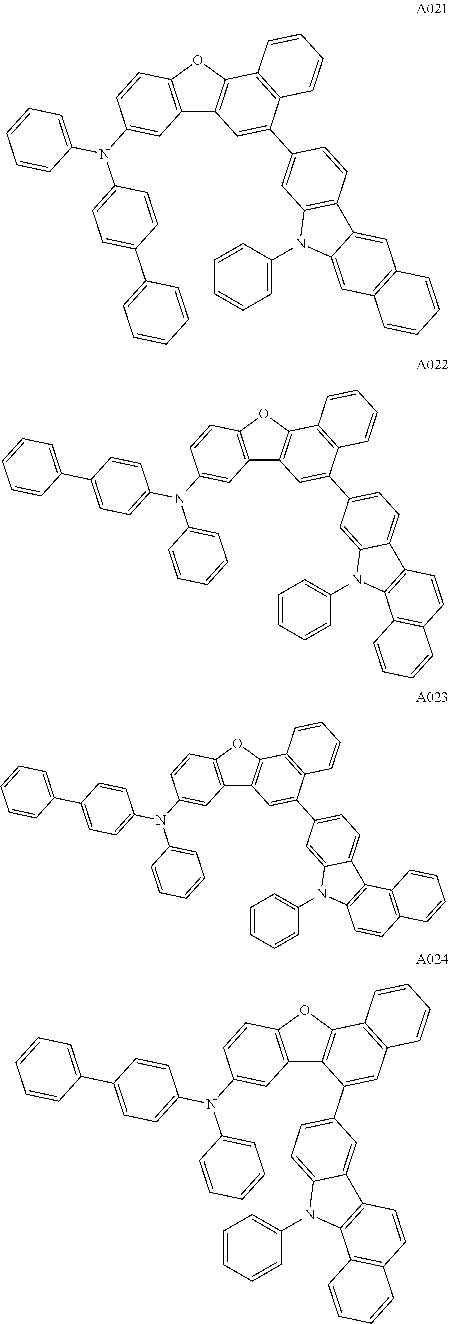 [SMILES: c1ccc(-c2ccc(N(c3ccccc3)c3ccc4oc5c6ccccc6c(-c6ccc7c8cc9ccccc9cc8n(-c8ccccc8)c7c6)cc5c4c3)cc2)cc1 ; c1ccc(-c2ccc(N(c3ccccc3)c3ccc4oc5c6ccccc6c(-c6ccc7c8ccc9ccccc9c8n(-c8ccccc8)c7c6)cc5c4c3)cc2)cc1 ; c1ccc(-c2ccc(N(c3ccccc3)c3ccc4oc5c6ccccc6c(-c6ccc7c8c9ccccc9ccc8n(-c8ccccc8)c7c6)cc5c4c3)cc2)cc1 ; c1ccc(-c2ccc(N(c3ccccc3)c3ccc4oc5c6ccccc6cc(-c6ccc7c(c6)c6ccc8ccccc8c6n7-c6ccccc6)c5c4c3)cc2)cc1]

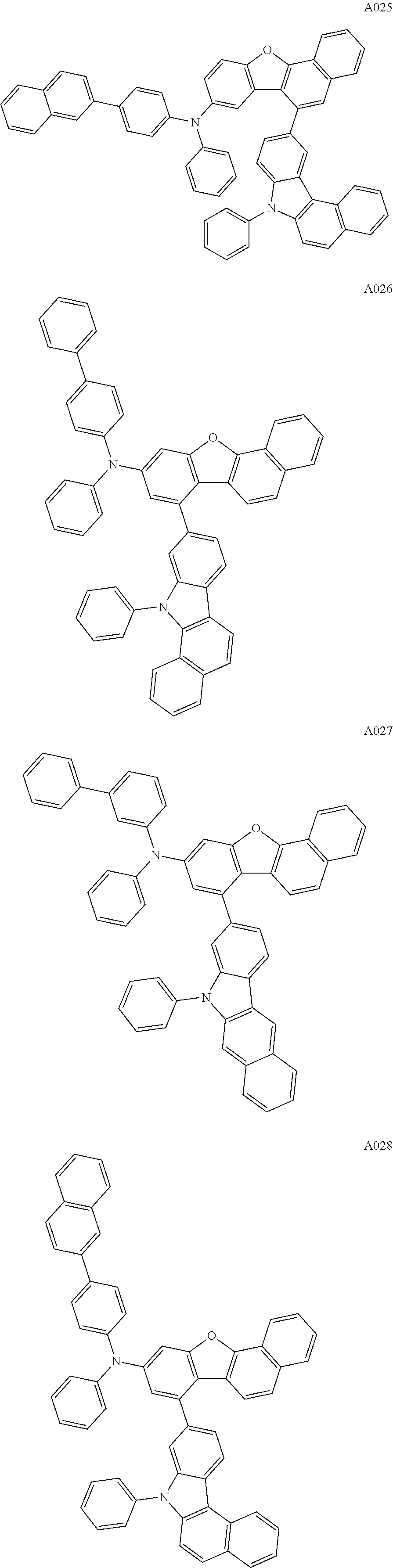 [SMILES: c1ccc(N(c2ccc(-c3ccc4ccccc4c3)cc2)c2ccc3oc4c5ccccc5cc(-c5ccc6c(c5)c5c7ccccc7ccc5n6-c5ccccc5)c4c3c2)cc1 ; c1ccc(-c2ccc(N(c3ccccc3)c3cc(-c4ccc5c6ccc7ccccc7c6n(-c6ccccc6)c5c4)c4c(c3)oc3c5ccccc5ccc34)cc2)cc1 ; c1ccc(-c2cccc(N(c3ccccc3)c3cc(-c4ccc5c6cc7ccccc7cc6n(-c6ccccc6)c5c4)c4c(c3)oc3c5ccccc5ccc34)c2)cc1 ; c1ccc(N(c2ccc(-c3ccc4ccccc4c3)cc2)c2cc(-c3ccc4c5c6ccccc6ccc5n(-c5ccccc5)c4c3)c3c(c2)oc2c4ccccc4ccc23)cc1]

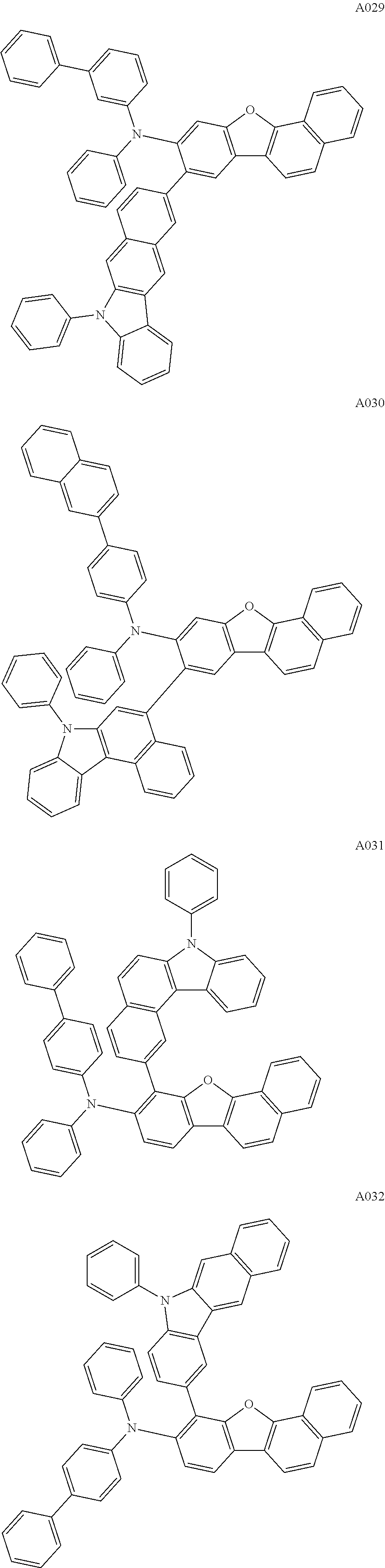 [SMILES: c1ccc(-c2cccc(N(c3ccccc3)c3cc4oc5c6ccccc6ccc5c4cc3-c3ccc4cc5c(cc4c3)c3ccccc3n5-c3ccccc3)c2)cc1 ; c1ccc(N(c2ccc(-c3ccc4ccccc4c3)cc2)c2cc3oc4c5ccccc5ccc4c3cc2-c2cc3c(c4ccccc24)c2ccccc2n3-c2ccccc2)cc1 ; c1ccc(-c2ccc(N(c3ccccc3)c3ccc4c(oc5c6ccccc6ccc45)c3-c3ccc4ccc5c(c4c3)c3ccccc3n5-c3ccccc3)cc2)cc1 ; c1ccc(-c2ccc(N(c3ccccc3)c3ccc4c(oc5c6ccccc6ccc45)c3-c3ccc4c(c3)c3cc5ccccc5cc3n4-c3ccccc3)cc2)cc1]

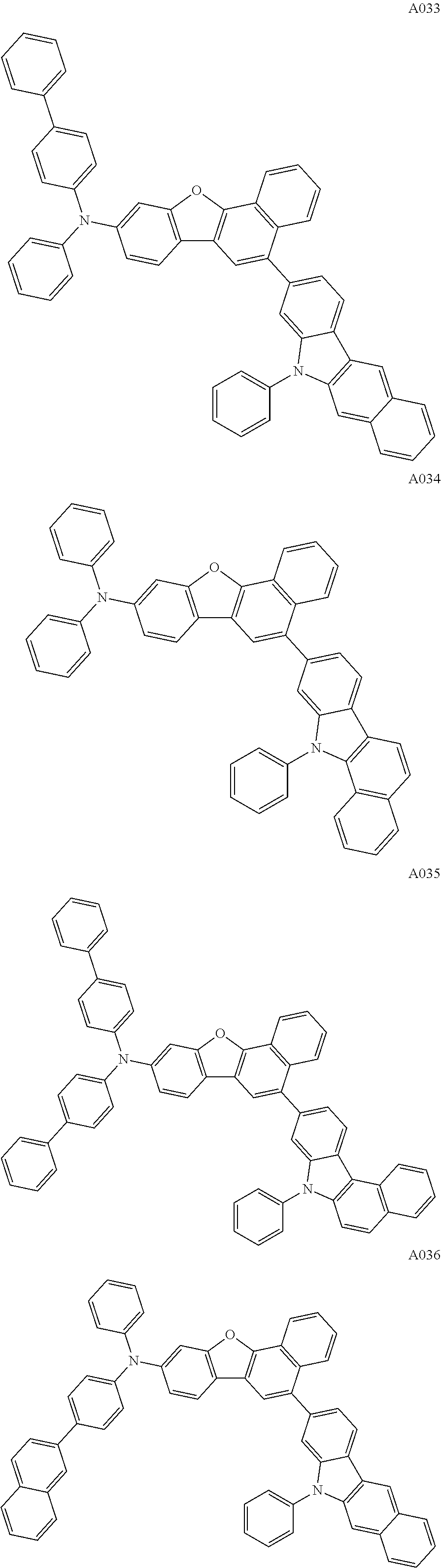 [SMILES: c1ccc(-c2ccc(N(c3ccccc3)c3ccc4c(c3)oc3c5ccccc5c(-c5ccc6c7cc8ccccc8cc7n(-c7ccccc7)c6c5)cc43)cc2)cc1 ; c1ccc(N(c2ccccc2)c2ccc3c(c2)oc2c4ccccc4c(-c4ccc5c6ccc7ccccc7c6n(-c6ccccc6)c5c4)cc32)cc1 ; c1ccc(-c2ccc(N(c3ccc(-c4ccccc4)cc3)c3ccc4c(c3)oc3c5ccccc5c(-c5ccc6c7c8ccccc8ccc7n(-c7ccccc7)c6c5)cc43)cc2)cc1 ; c1ccc(N(c2ccc(-c3ccc4ccccc4c3)cc2)c2ccc3c(c2)oc2c4ccccc4c(-c4ccc5c6cc7ccccc7cc6n(-c6ccccc6)c5c4)cc32)cc1]

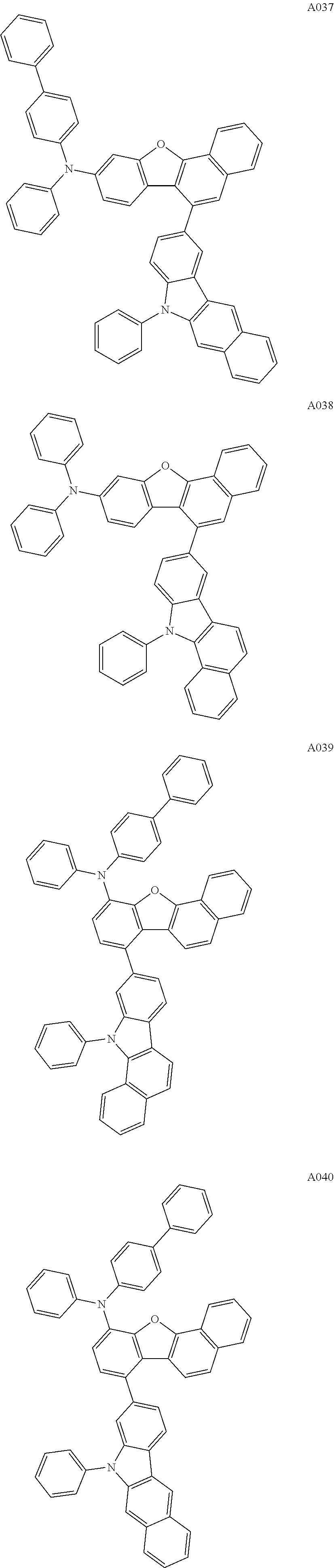 [SMILES: c1ccc(-c2ccc(N(c3ccccc3)c3ccc4c(c3)oc3c5ccccc5cc(-c5ccc6c(c5)c5cc7ccccc7cc5n6-c5ccccc5)c43)cc2)cc1 ; c1ccc(N(c2ccccc2)c2ccc3c(c2)oc2c4ccccc4cc(-c4ccc5c(c4)c4ccc6ccccc6c4n5-c4ccccc4)c32)cc1 ; c1ccc(-c2ccc(N(c3ccccc3)c3ccc(-c4ccc5c6ccc7ccccc7c6n(-c6ccccc6)c5c4)c4c3oc3c5ccccc5ccc34)cc2)cc1 ; c1ccc(-c2ccc(N(c3ccccc3)c3ccc(-c4ccc5c6cc7ccccc7cc6n(-c6ccccc6)c5c4)c4c3oc3c5ccccc5ccc34)cc2)cc1]

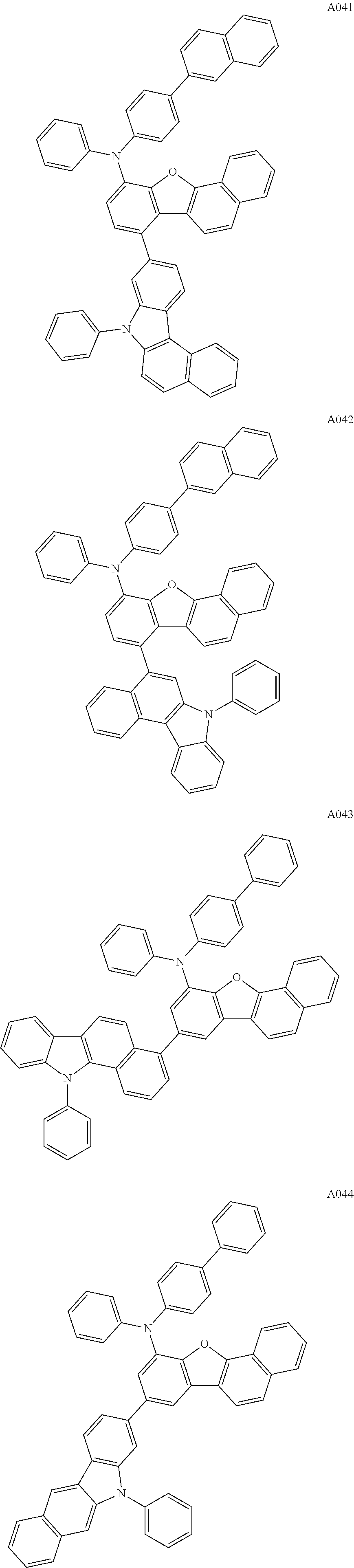 [SMILES: c1ccc(N(c2ccc(-c3ccc4ccccc4c3)cc2)c2ccc(-c3ccc4c5c6ccccc6ccc5n(-c5ccccc5)c4c3)c3c2oc2c4ccccc4ccc23)cc1 ; c1ccc(N(c2ccc(-c3ccc4ccccc4c3)cc2)c2ccc(-c3cc4c(c5ccccc35)c3ccccc3n4-c3ccccc3)c3c2oc2c4ccccc4ccc23)cc1 ; c1ccc(-c2ccc(N(c3ccccc3)c3cc(-c4cccc5c4ccc4c6ccccc6n(-c6ccccc6)c54)cc4c3oc3c5ccccc5ccc43)cc2)cc1 ; c1ccc(-c2ccc(N(c3ccccc3)c3cc(-c4ccc5c6cc7ccccc7cc6n(-c6ccccc6)c5c4)cc4c3oc3c5ccccc5ccc43)cc2)cc1]

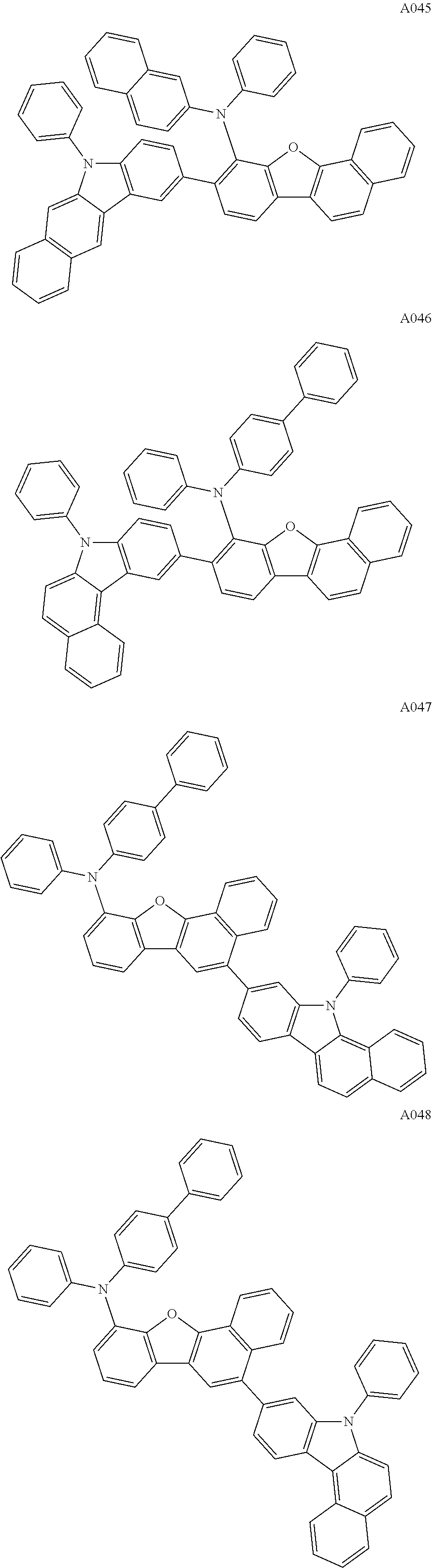 [SMILES: c1ccc(N(c2ccc3ccccc3c2)c2c(-c3ccc4c(c3)c3cc5ccccc5cc3n4-c3ccccc3)ccc3c2oc2c4ccccc4ccc32)cc1 ; c1ccc(-c2ccc(N(c3ccccc3)c3c(-c4ccc5c(c4)c4c6ccccc6ccc4n5-c4ccccc4)ccc4c3oc3c5ccccc5ccc43)cc2)cc1 ; c1ccc(-c2ccc(N(c3ccccc3)c3cccc4c3oc3c5ccccc5c(-c5ccc6c7ccc8ccccc8c7n(-c7ccccc7)c6c5)cc43)cc2)cc1 ; c1ccc(-c2ccc(N(c3ccccc3)c3cccc4c3oc3c5ccccc5c(-c5ccc6c7c8ccccc8ccc7n(-c7ccccc7)c6c5)cc43)cc2)cc1]

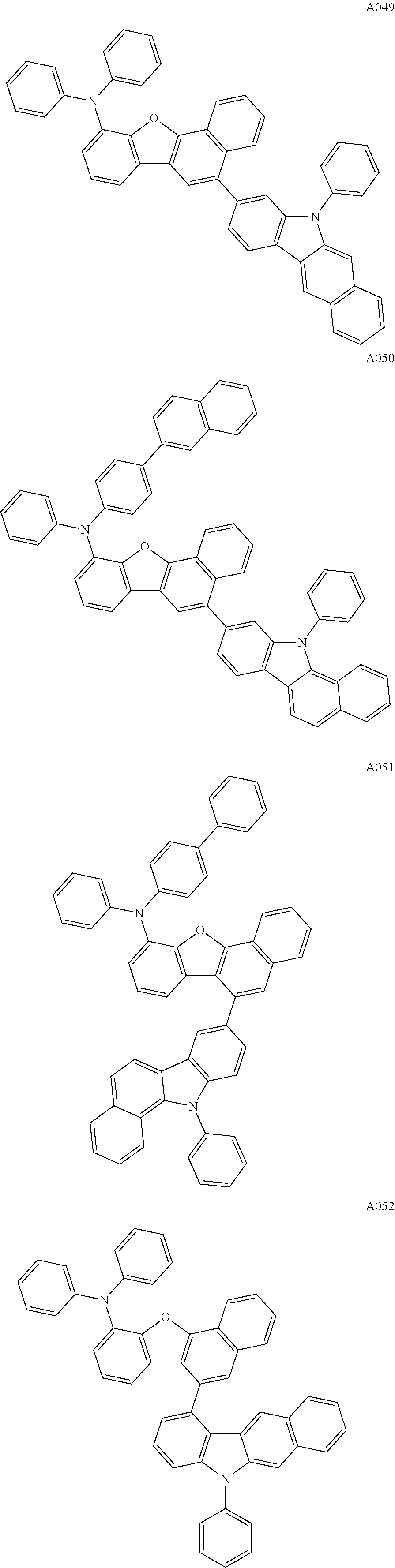 [SMILES: c1ccc(N(c2ccccc2)c2cccc3c2oc2c4ccccc4c(-c4ccc5c6cc7ccccc7cc6n(-c6ccccc6)c5c4)cc32)cc1 ; c1ccc(N(c2ccc(-c3ccc4ccccc4c3)cc2)c2cccc3c2oc2c4ccccc4c(-c4ccc5c6ccc7ccccc7c6n(-c6ccccc6)c5c4)cc32)cc1 ; c1ccc(-c2ccc(N(c3ccccc3)c3cccc4c3oc3c5ccccc5cc(-c5ccc6c(c5)c5ccc7ccccc7c5n6-c5ccccc5)c43)cc2)cc1 ; c1ccc(N(c2ccccc2)c2cccc3c2oc2c4ccccc4cc(-c4cccc5c4c4cc6ccccc6cc4n5-c4ccccc4)c32)cc1]

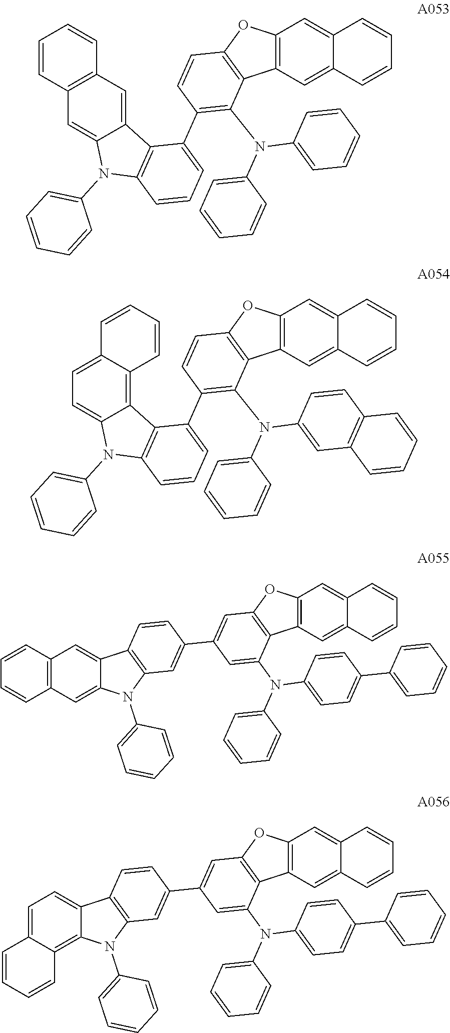 [SMILES: c1ccc(N(c2ccccc2)c2c(-c3cccc4c3c3cc5ccccc5cc3n4-c3ccccc3)ccc3oc4cc5ccccc5cc4c23)cc1 ; c1ccc(N(c2ccc3ccccc3c2)c2c(-c3cccc4c3c3c5ccccc5ccc3n4-c3ccccc3)ccc3oc4cc5ccccc5cc4c23)cc1 ; c1ccc(-c2ccc(N(c3ccccc3)c3cc(-c4ccc5c6cc7ccccc7cc6n(-c6ccccc6)c5c4)cc4oc5cc6ccccc6cc5c34)cc2)cc1 ; c1ccc(-c2ccc(N(c3ccccc3)c3cc(-c4ccc5c6ccc7ccccc7c6n(-c6ccccc6)c5c4)cc4oc5cc6ccccc6cc5c34)cc2)cc1]

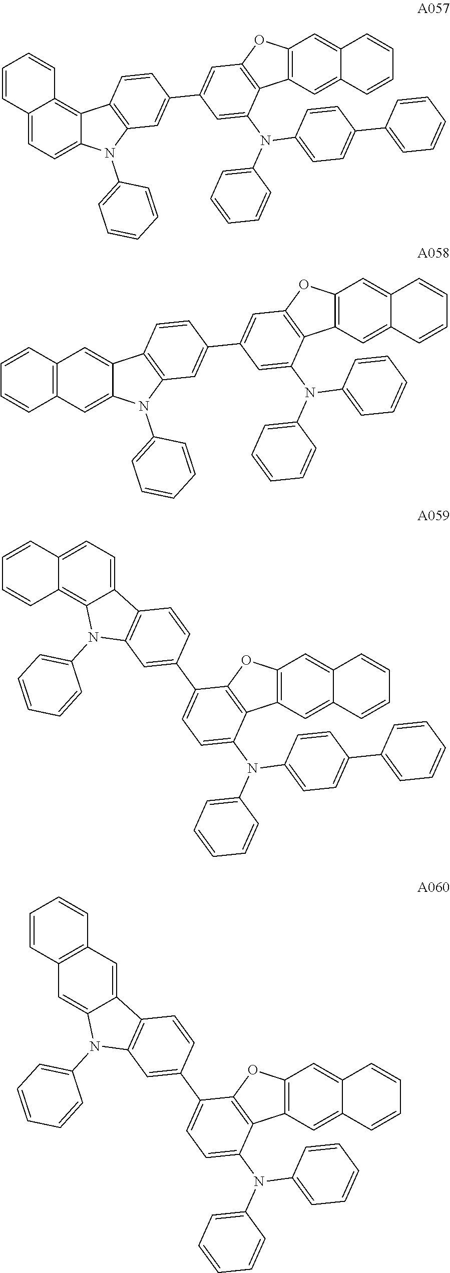 [SMILES: c1ccc(-c2ccc(N(c3ccccc3)c3cc(-c4ccc5c6c7ccccc7ccc6n(-c6ccccc6)c5c4)cc4oc5cc6ccccc6cc5c34)cc2)cc1 ; c1ccc(N(c2ccccc2)c2cc(-c3ccc4c5cc6ccccc6cc5n(-c5ccccc5)c4c3)cc3oc4cc5ccccc5cc4c23)cc1 ; c1ccc(-c2ccc(N(c3ccccc3)c3ccc(-c4ccc5c6ccc7ccccc7c6n(-c6ccccc6)c5c4)c4oc5cc6ccccc6cc5c34)cc2)cc1 ; c1ccc(N(c2ccccc2)c2ccc(-c3ccc4c5cc6ccccc6cc5n(-c5ccccc5)c4c3)c3oc4cc5ccccc5cc4c23)cc1]

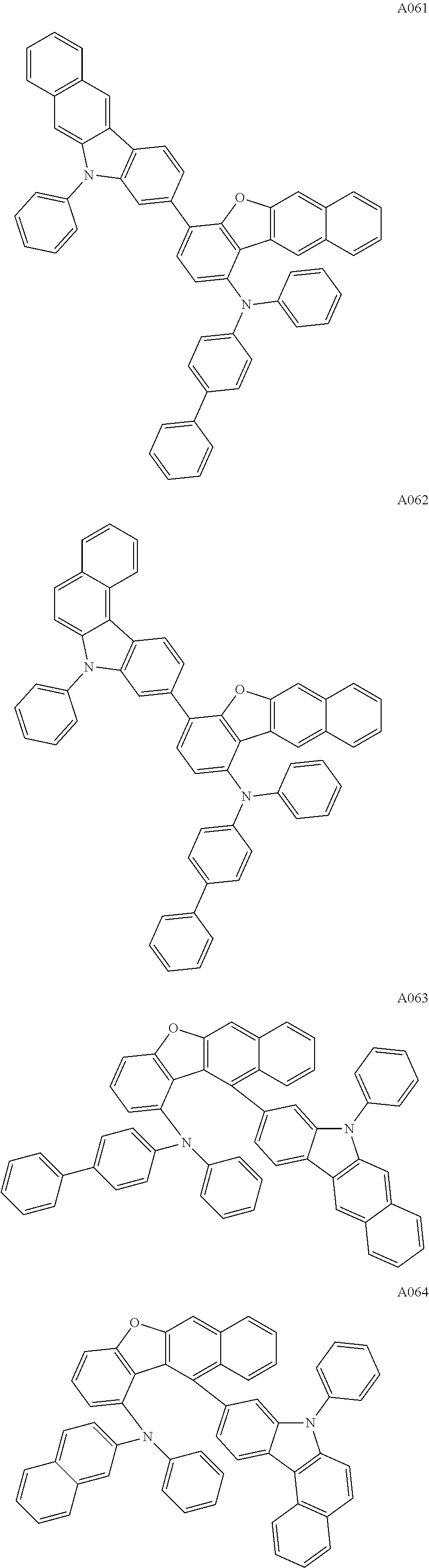 [SMILES: c1ccc(-c2ccc(N(c3ccccc3)c3ccc(-c4ccc5c6cc7ccccc7cc6n(-c6ccccc6)c5c4)c4oc5cc6ccccc6cc5c34)cc2)cc1 ; c1ccc(-c2ccc(N(c3ccccc3)c3ccc(-c4ccc5c6c7ccccc7ccc6n(-c6ccccc6)c5c4)c4oc5cc6ccccc6cc5c34)cc2)cc1 ; c1ccc(-c2ccc(N(c3ccccc3)c3cccc4oc5cc6ccccc6c(-c6ccc7c8cc9ccccc9cc8n(-c8ccccc8)c7c6)c5c34)cc2)cc1 ; c1ccc(N(c2ccc3ccccc3c2)c2cccc3oc4cc5ccccc5c(-c5ccc6c7c8ccccc8ccc7n(-c7ccccc7)c6c5)c4c23)cc1]

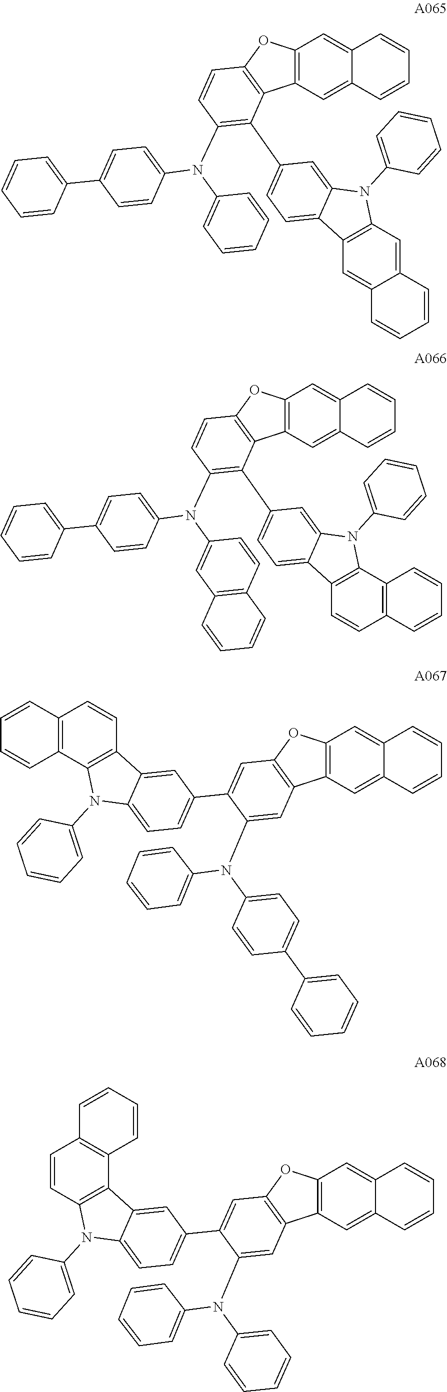 [SMILES: c1ccc(-c2ccc(N(c3ccccc3)c3ccc4oc5cc6ccccc6cc5c4c3-c3ccc4c5cc6ccccc6cc5n(-c5ccccc5)c4c3)cc2)cc1 ; c1ccc(-c2ccc(N(c3ccc4ccccc4c3)c3ccc4oc5cc6ccccc6cc5c4c3-c3ccc4c5ccc6ccccc6c5n(-c5ccccc5)c4c3)cc2)cc1 ; c1ccc(-c2ccc(N(c3ccccc3)c3cc4c(cc3-c3ccc5c(c3)c3ccc6ccccc6c3n5-c3ccccc3)oc3cc5ccccc5cc34)cc2)cc1 ; c1ccc(N(c2ccccc2)c2cc3c(cc2-c2ccc4c(c2)c2c5ccccc5ccc2n4-c2ccccc2)oc2cc4ccccc4cc23)cc1]

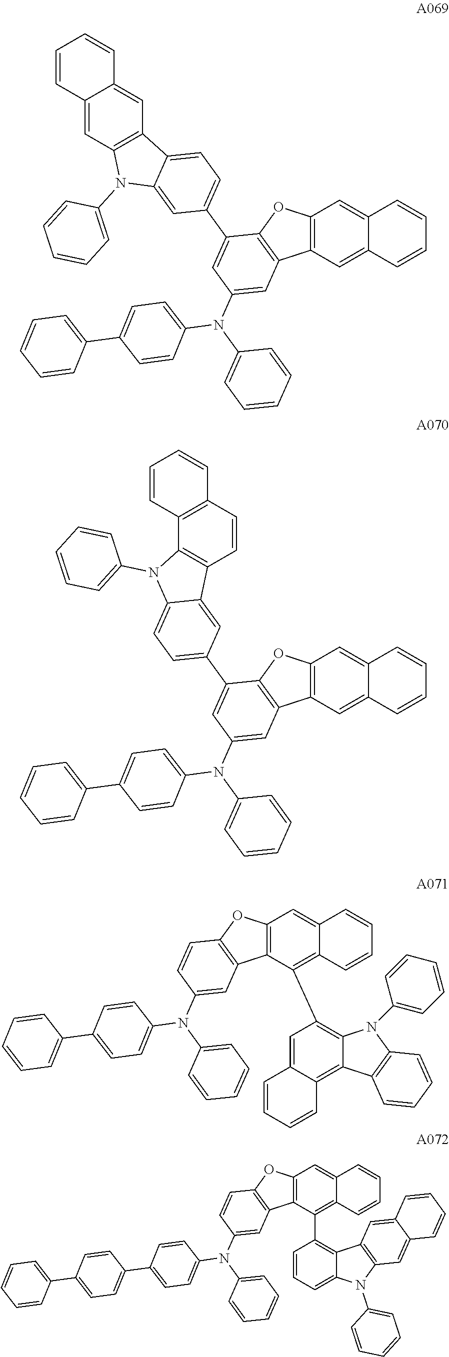 [SMILES: c1ccc(-c2ccc(N(c3ccccc3)c3cc(-c4ccc5c6cc7ccccc7cc6n(-c6ccccc6)c5c4)c4oc5cc6ccccc6cc5c4c3)cc2)cc1 ; c1ccc(-c2ccc(N(c3ccccc3)c3cc(-c4ccc5c(c4)c4ccc6ccccc6c4n5-c4ccccc4)c4oc5cc6ccccc6cc5c4c3)cc2)cc1 ; c1ccc(-c2ccc(N(c3ccccc3)c3ccc4oc5cc6ccccc6c(-c6cc7ccccc7c7c8ccccc8n(-c8ccccc8)c67)c5c4c3)cc2)cc1 ; c1ccc(-c2ccc(-c3ccc(N(c4ccccc4)c4ccc5oc6cc7ccccc7c(-c7cccc8c7c7cc9ccccc9cc7n8-c7ccccc7)c6c5c4)cc3)cc2)cc1]

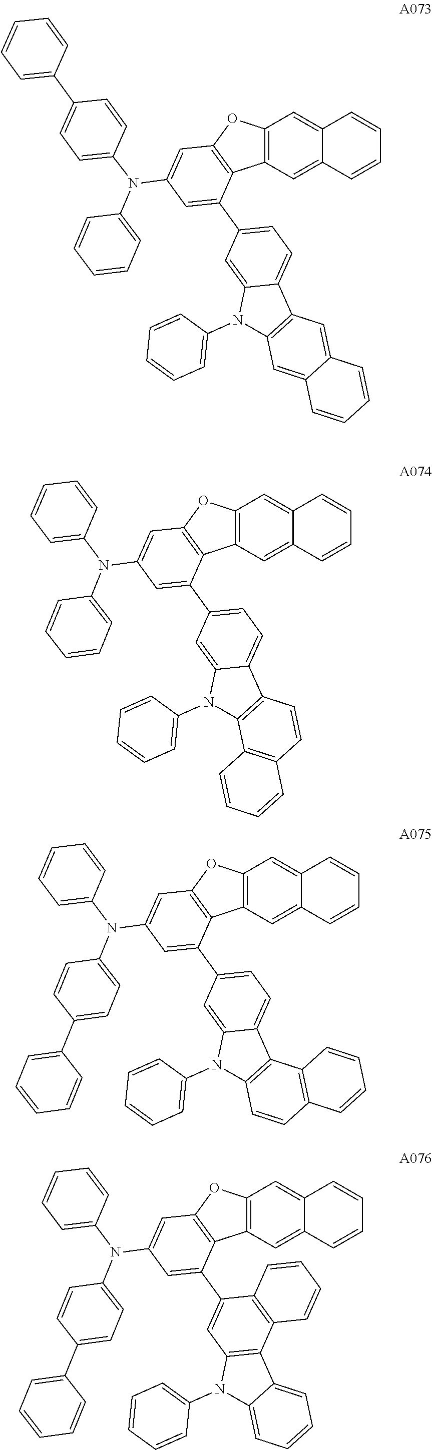

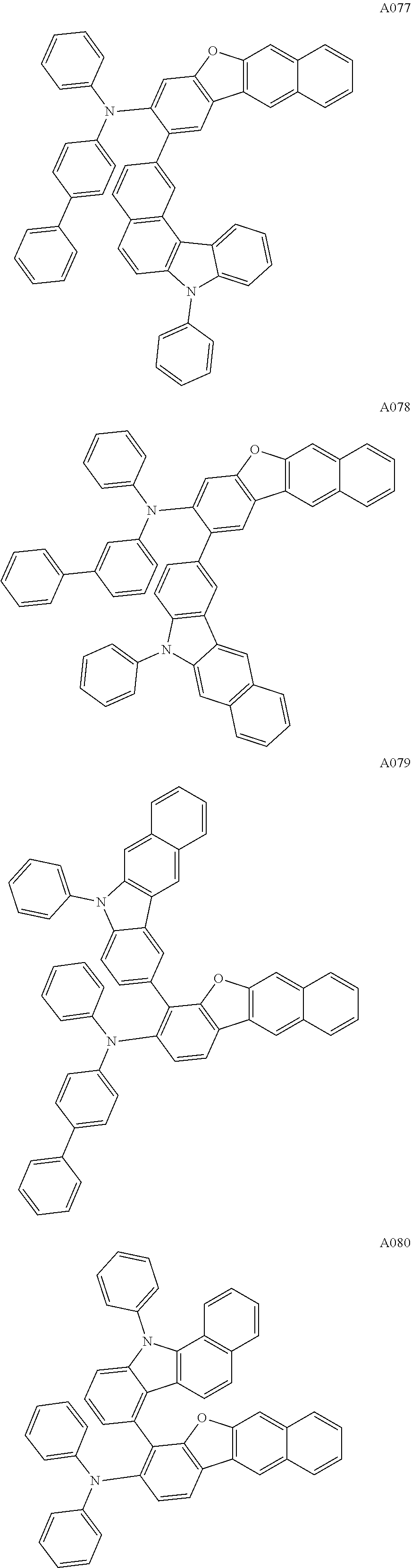 [SMILES: c1ccc(-c2ccc(N(c3ccccc3)c3cc4oc5cc6ccccc6cc5c4cc3-c3ccc4ccc5c(c4c3)c3ccccc3n5-c3ccccc3)cc2)cc1 ; c1ccc(-c2cccc(N(c3ccccc3)c3cc4oc5cc6ccccc6cc5c4cc3-c3ccc4c(c3)c3cc5ccccc5cc3n4-c3ccccc3)c2)cc1 ; c1ccc(-c2ccc(N(c3ccccc3)c3ccc4c(oc5cc6ccccc6cc54)c3-c3ccc4c(c3)c3cc5ccccc5cc3n4-c3ccccc3)cc2)cc1 ; c1ccc(N(c2ccccc2)c2ccc3c(oc4cc5ccccc5cc43)c2-c2cccc3c2c2ccc4ccccc4c2n3-c2ccccc2)cc1]

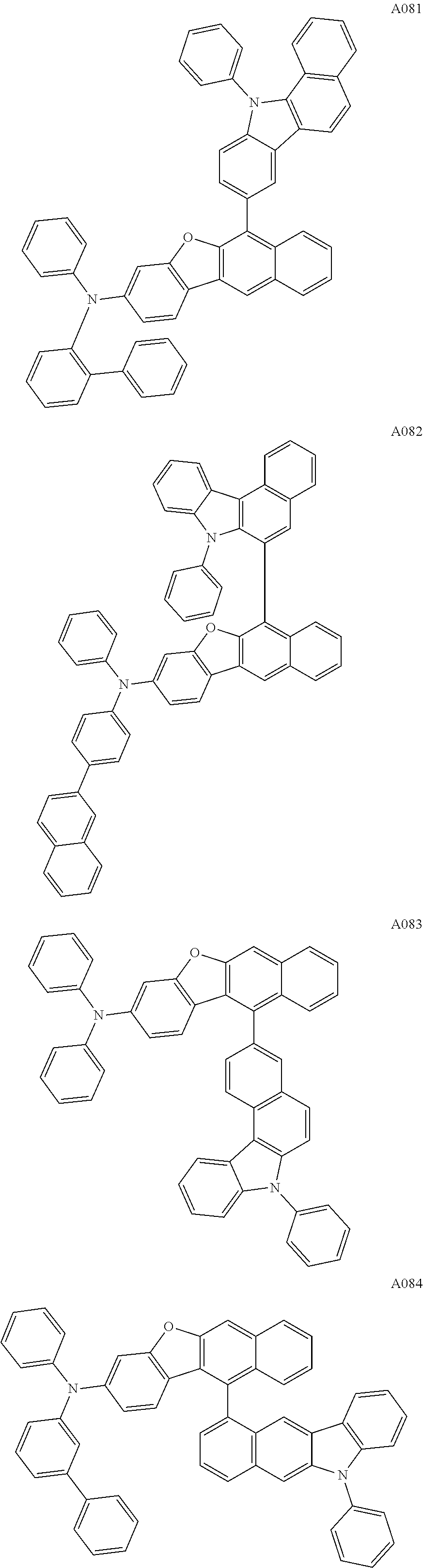

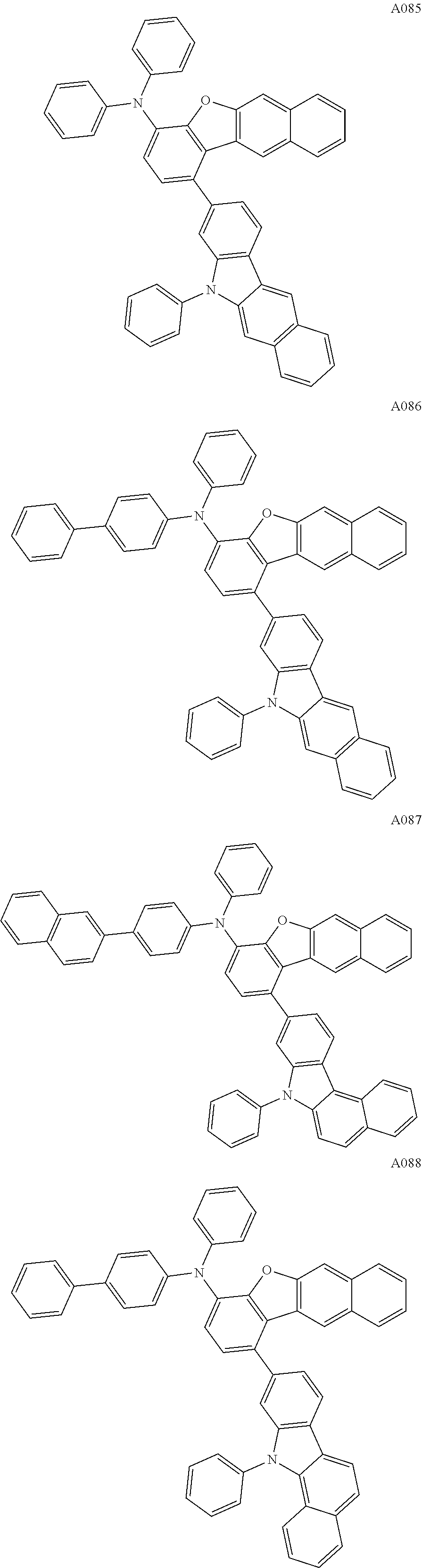

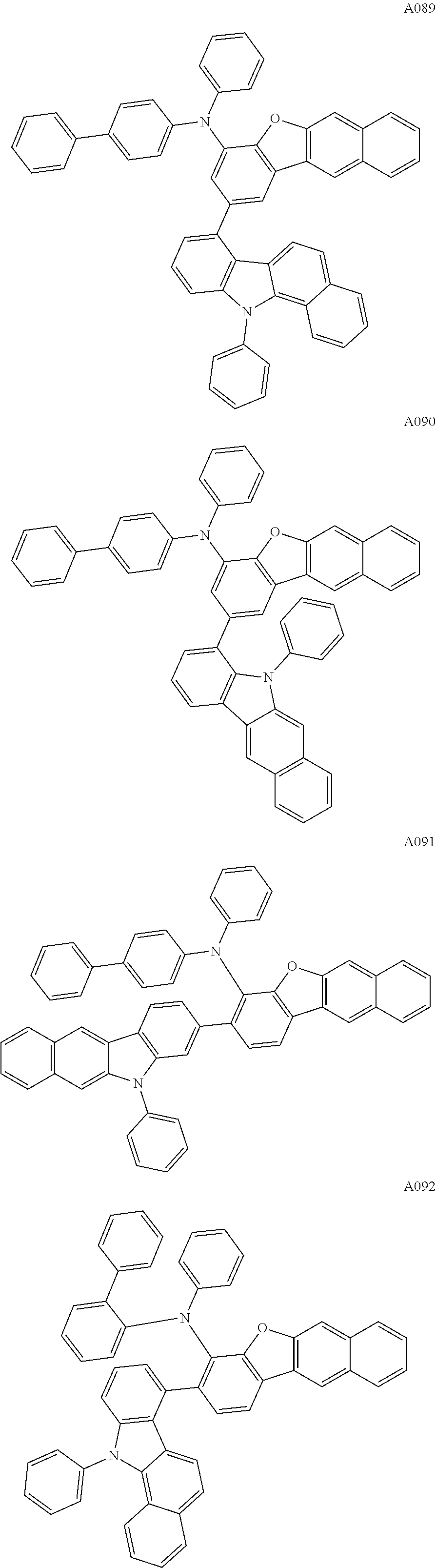

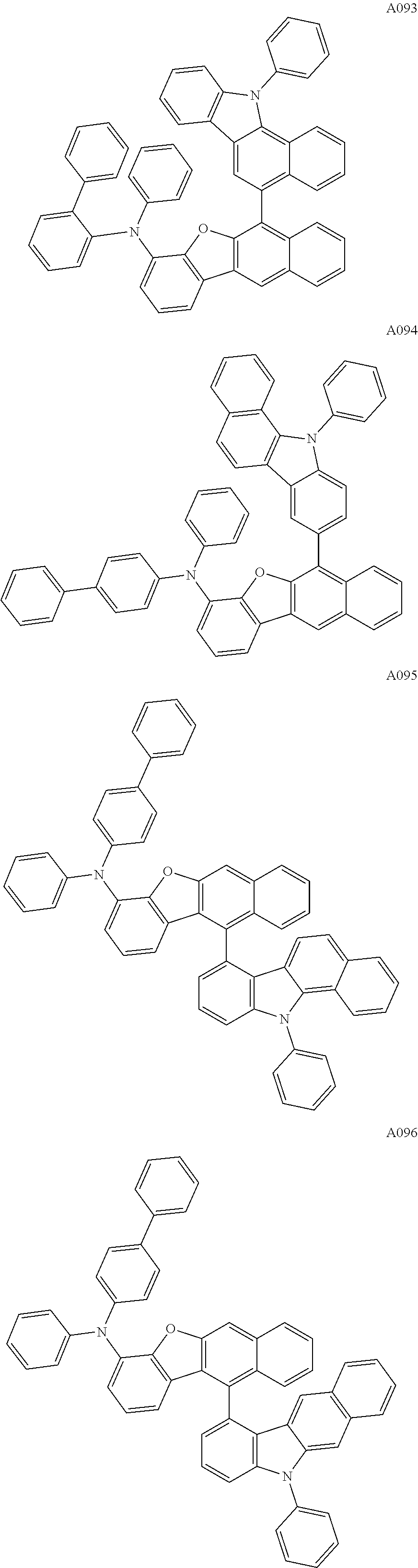 [SMILES: c1ccc(-c2ccccc2N(c2ccccc2)c2cccc3c2oc2c(-c4cc5c6ccccc6n(-c6ccccc6)c5c5ccccc45)c4ccccc4cc23)cc1 ; c1ccc(-c2ccc(N(c3ccccc3)c3cccc4c3oc3c(-c5ccc6c(c5)c5ccc7ccccc7c5n6-c5ccccc5)c5ccccc5cc34)cc2)cc1 ; c1ccc(-c2ccc(N(c3ccccc3)c3cccc4c3oc3cc5ccccc5c(-c5cccc6c5c5ccc7ccccc7c5n6-c5ccccc5)c34)cc2)cc1 ; c1ccc(-c2ccc(N(c3ccccc3)c3cccc4c3oc3cc5ccccc5c(-c5cccc6c5c5cc7ccccc7cc5n6-c5ccccc5)c34)cc2)cc1]

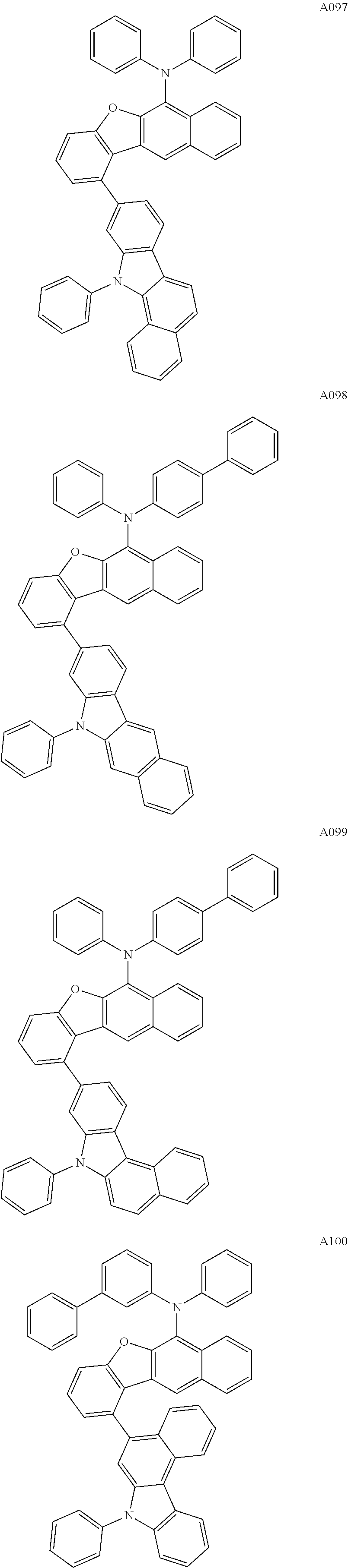

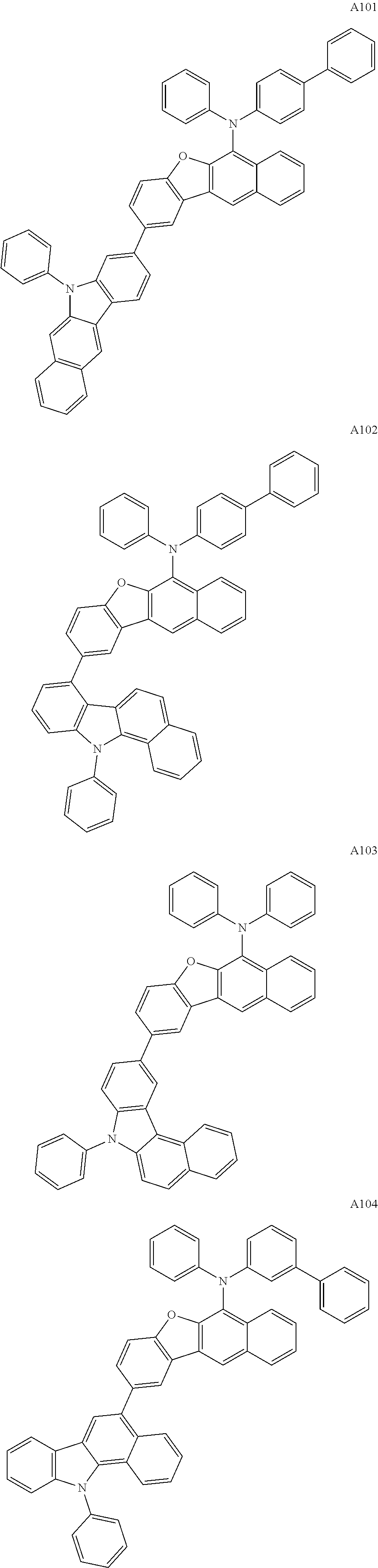

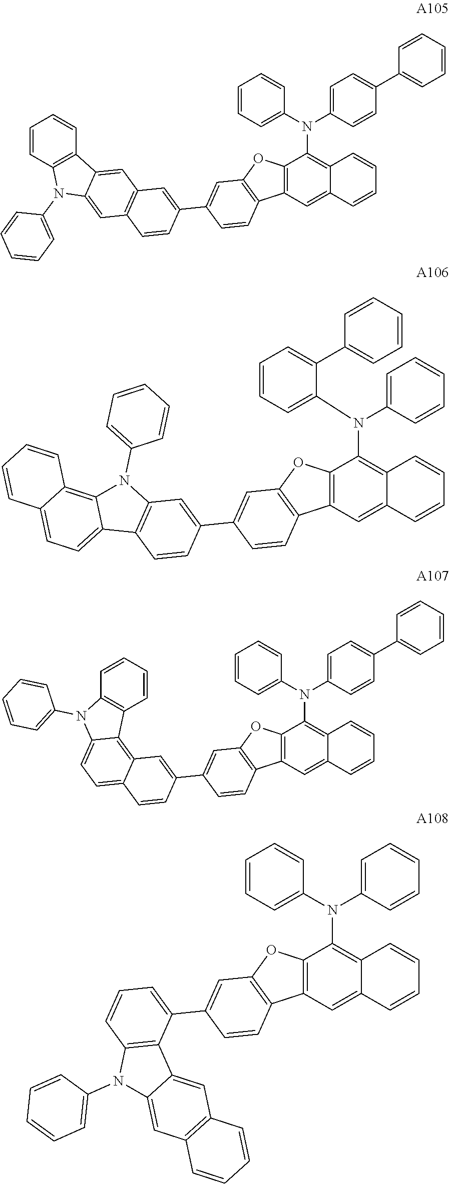 [SMILES: c1ccc(-c2ccc(N(c3ccccc3)c3c4ccccc4cc4c3oc3cc(-c5ccc6cc7c(cc6c5)c5ccccc5n7-c5ccccc5)ccc34)cc2)cc1 ; c1ccc(-c2ccccc2N(c2ccccc2)c2c3ccccc3cc3c2oc2cc(-c4ccc5c6ccc7ccccc7c6n(-c6ccccc6)c5c4)ccc23)cc1 ; c1ccc(-c2ccc(N(c3ccccc3)c3c4ccccc4cc4c3oc3cc(-c5ccc6ccc7c(c6c5)c5ccccc5n7-c5ccccc5)ccc34)cc2)cc1 ; c1ccc(N(c2ccccc2)c2c3ccccc3cc3c2oc2cc(-c4cccc5c4c4cc6ccccc6cc4n5-c4ccccc4)ccc23)cc1]

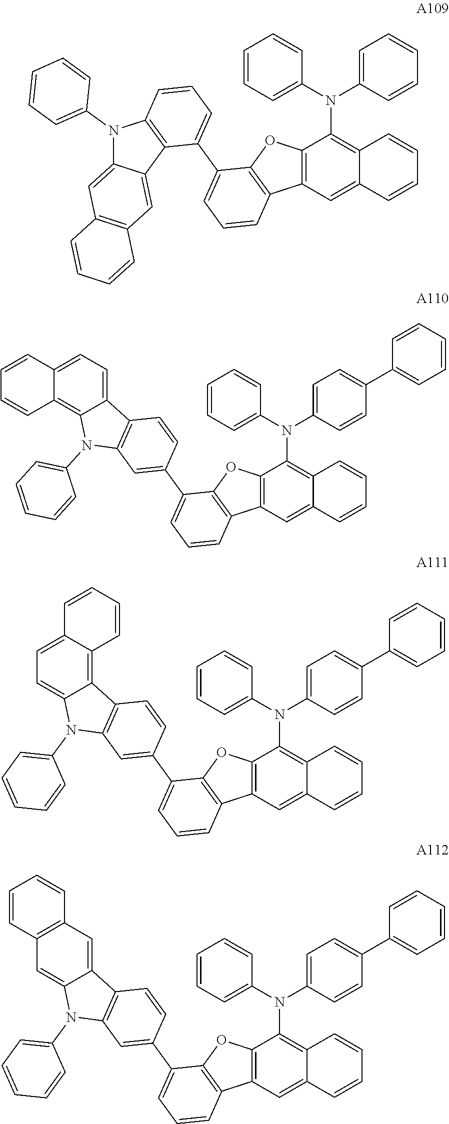 [SMILES: c1ccc(N(c2ccccc2)c2c3ccccc3cc3c2oc2c(-c4cccc5c4c4cc6ccccc6cc4n5-c4ccccc4)cccc23)cc1 ; c1ccc(-c2ccc(N(c3ccccc3)c3c4ccccc4cc4c3oc3c(-c5ccc6c7ccc8ccccc8c7n(-c7ccccc7)c6c5)cccc34)cc2)cc1 ; c1ccc(-c2ccc(N(c3ccccc3)c3c4ccccc4cc4c3oc3c(-c5ccc6c7c8ccccc8ccc7n(-c7ccccc7)c6c5)cccc34)cc2)cc1 ; c1ccc(-c2ccc(N(c3ccccc3)c3c4ccccc4cc4c3oc3c(-c5ccc6c7cc8ccccc8cc7n(-c7ccccc7)c6c5)cccc34)cc2)cc1]

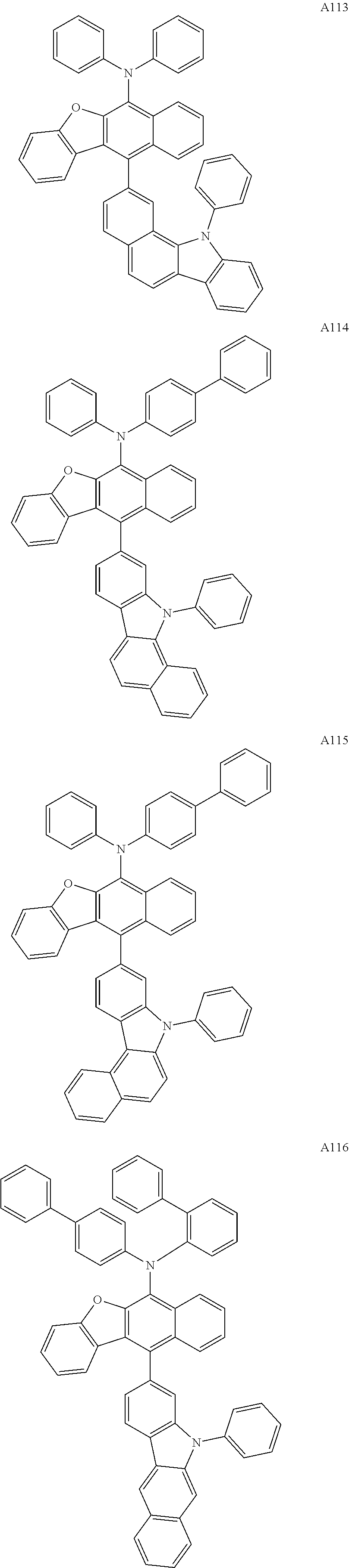

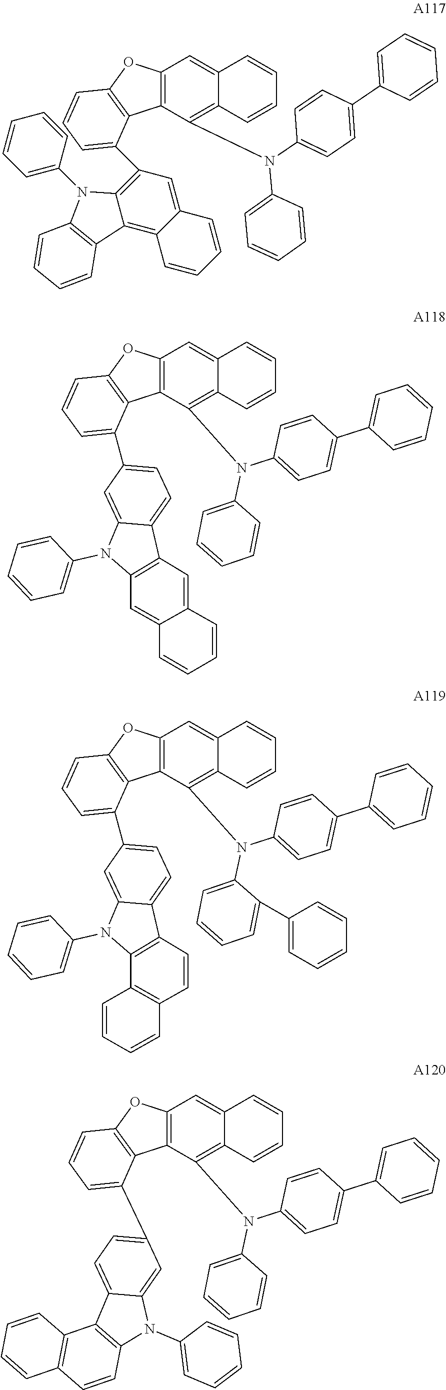 [SMILES: c1ccc(-c2ccc(N(c3ccccc3)c3c4ccccc4cc4oc5cccc(-c6cc7ccccc7c7c8ccccc8n(-c8ccccc8)c67)c5c34)cc2)cc1 ; c1ccc(-c2ccc(N(c3ccccc3)c3c4ccccc4cc4oc5cccc(-c6ccc7c8cc9ccccc9cc8n(-c8ccccc8)c7c6)c5c34)cc2)cc1 ; c1ccc(-c2ccc(N(c3ccccc3-c3ccccc3)c3c4ccccc4cc4oc5cccc(-c6ccc7c8ccc9ccccc9c8n(-c8ccccc8)c7c6)c5c34)cc2)cc1 ; c1ccc(-c2ccc(N(c3ccccc3)c3c4ccccc4cc4oc5cccc(-c6ccc7c8c9ccccc9ccc8n(-c8ccccc8)c7c6)c5c34)cc2)cc1]

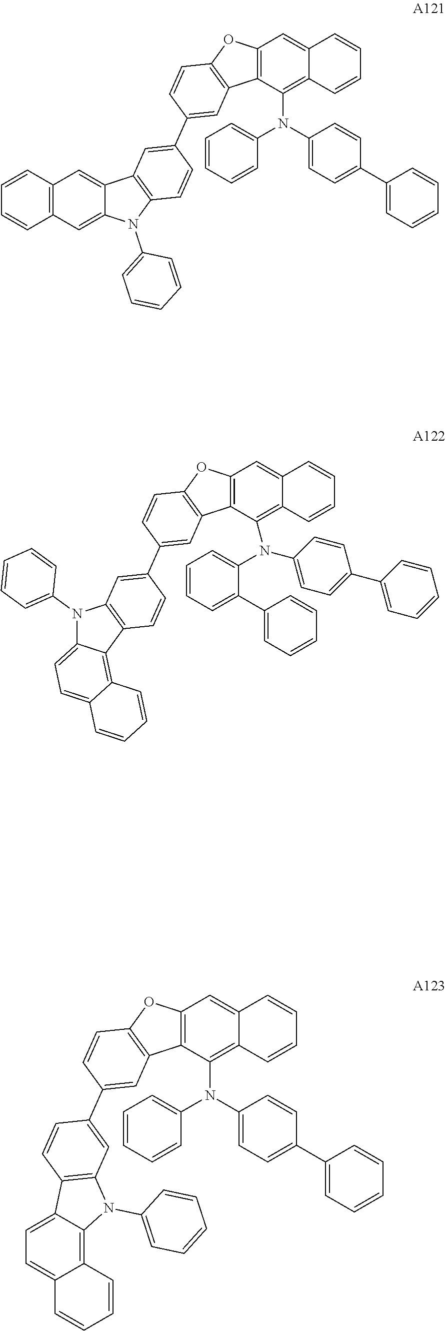 [SMILES: c1ccc(-c2ccc(N(c3ccccc3)c3c4ccccc4cc4oc5ccc(-c6ccc7c(c6)c6cc8ccccc8cc6n7-c6ccccc6)cc5c34)cc2)cc1 ; c1ccc(-c2ccc(N(c3ccccc3-c3ccccc3)c3c4ccccc4cc4oc5ccc(-c6ccc7c8c9ccccc9ccc8n(-c8ccccc8)c7c6)cc5c34)cc2)cc1 ; c1ccc(-c2ccc(N(c3ccccc3)c3c4ccccc4cc4oc5ccc(-c6ccc7c8ccc9ccccc9c8n(-c8ccccc8)c7c6)cc5c34)cc2)cc1]

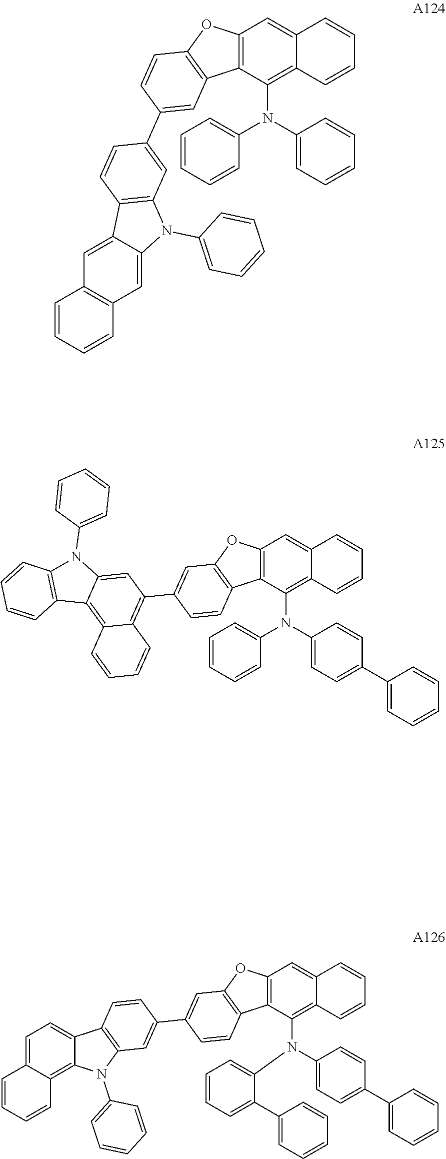 [SMILES: c1ccc(N(c2ccccc2)c2c3ccccc3cc3oc4ccc(-c5ccc6c7cc8ccccc8cc7n(-c7ccccc7)c6c5)cc4c23)cc1 ; c1ccc(-c2ccc(N(c3ccccc3)c3c4ccccc4cc4oc5cc(-c6cc7c(c8ccccc68)c6ccccc6n7-c6ccccc6)ccc5c34)cc2)cc1 ; c1ccc(-c2ccc(N(c3ccccc3-c3ccccc3)c3c4ccccc4cc4oc5cc(-c6ccc7c8ccc9ccccc9c8n(-c8ccccc8)c7c6)ccc5c34)cc2)cc1]

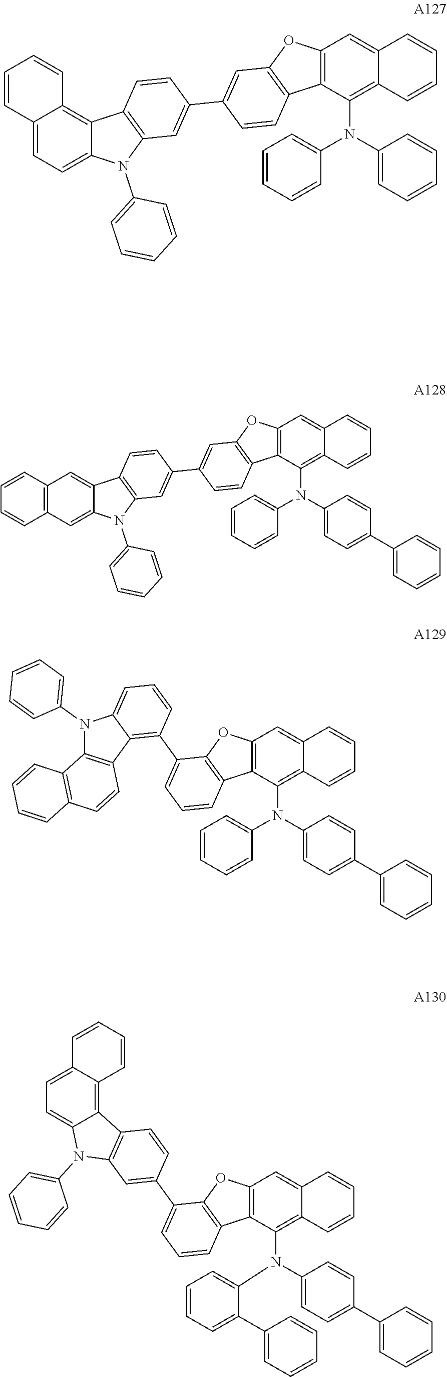 [SMILES: c1ccc(N(c2ccccc2)c2c3ccccc3cc3oc4cc(-c5ccc6c7c8ccccc8ccc7n(-c7ccccc7)c6c5)ccc4c23)cc1 ; c1ccc(-c2ccc(N(c3ccccc3)c3c4ccccc4cc4oc5cc(-c6ccc7c8cc9ccccc9cc8n(-c8ccccc8)c7c6)ccc5c34)cc2)cc1 ; c1ccc(-c2ccc(N(c3ccccc3)c3c4ccccc4cc4oc5c(-c6cccc7c6c6ccc8ccccc8c6n7-c6ccccc6)cccc5c34)cc2)cc1 ; c1ccc(-c2ccc(N(c3ccccc3-c3ccccc3)c3c4ccccc4cc4oc5c(-c6ccc7c8c9ccccc9ccc8n(-c8ccccc8)c7c6)cccc5c34)cc2)cc1]

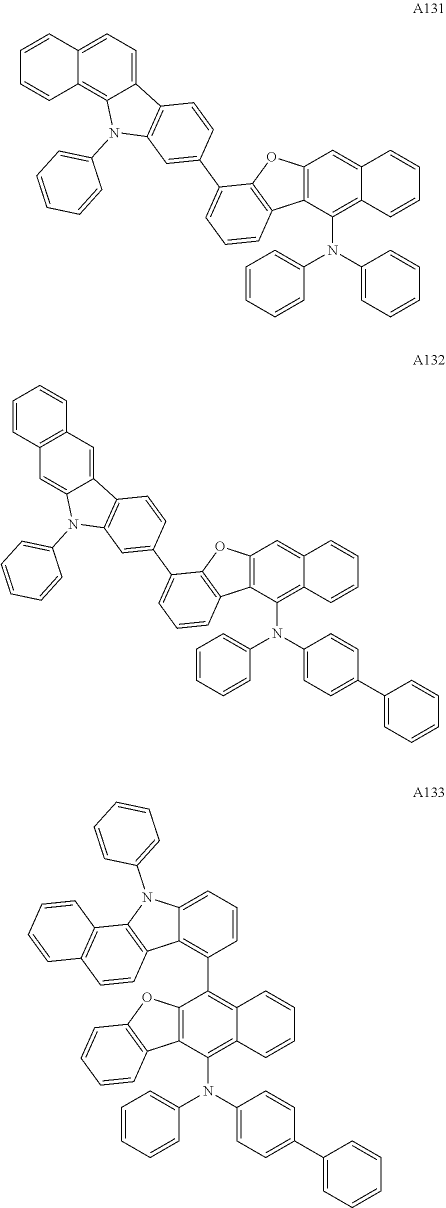 [SMILES: c1ccc(N(c2ccccc2)c2c3ccccc3cc3oc4c(-c5ccc6c7ccc8ccccc8c7n(-c7ccccc7)c6c5)cccc4c23)cc1 ; c1ccc(-c2ccc(N(c3ccccc3)c3c4ccccc4cc4oc5c(-c6ccc7c8cc9ccccc9cc8n(-c8ccccc8)c7c6)cccc5c34)cc2)cc1 ; c1ccc(-c2ccc(N(c3ccccc3)c3c4ccccc4c(-c4cccc5c4c4ccc6ccccc6c4n5-c4ccccc4)c4oc5ccccc5c34)cc2)cc1]

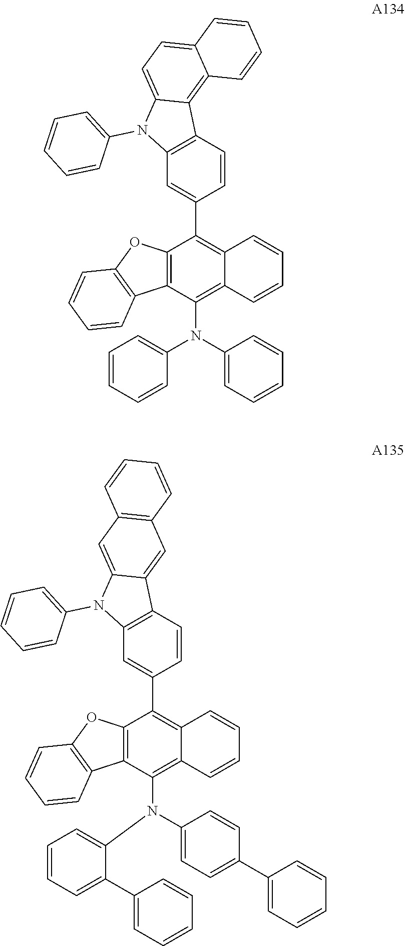 [SMILES: c1ccc(N(c2ccccc2)c2c3ccccc3c(-c3ccc4c5c6ccccc6ccc5n(-c5ccccc5)c4c3)c3oc4ccccc4c23)cc1 ; c1ccc(-c2ccc(N(c3ccccc3-c3ccccc3)c3c4ccccc4c(-c4ccc5c6cc7ccccc7cc6n(-c6ccccc6)c5c4)c4oc5ccccc5c34)cc2)cc1]

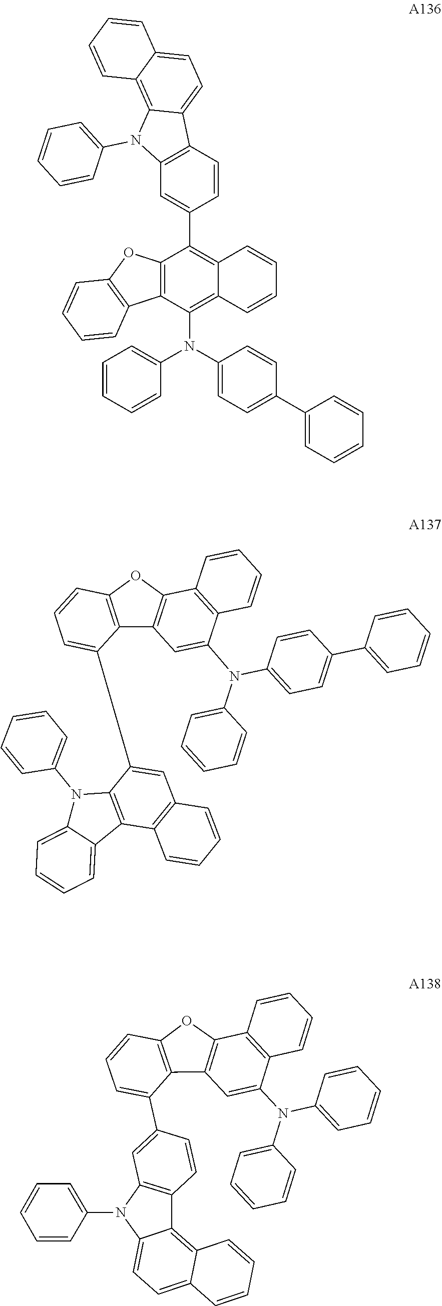 [SMILES: c1ccc(-c2ccc(N(c3ccccc3)c3c4ccccc4c(-c4ccc5c6ccc7ccccc7c6n(-c6ccccc6)c5c4)c4oc5ccccc5c34)cc2)cc1 ; c1ccc(-c2ccc(N(c3ccccc3)c3cc4c(oc5cccc(-c6cc7ccccc7c7c8ccccc8n(-c8ccccc8)c67)c54)c4ccccc34)cc2)cc1 ; c1ccc(N(c2ccccc2)c2cc3c(oc4cccc(-c5ccc6c7c8ccccc8ccc7n(-c7ccccc7)c6c5)c43)c3ccccc23)cc1]

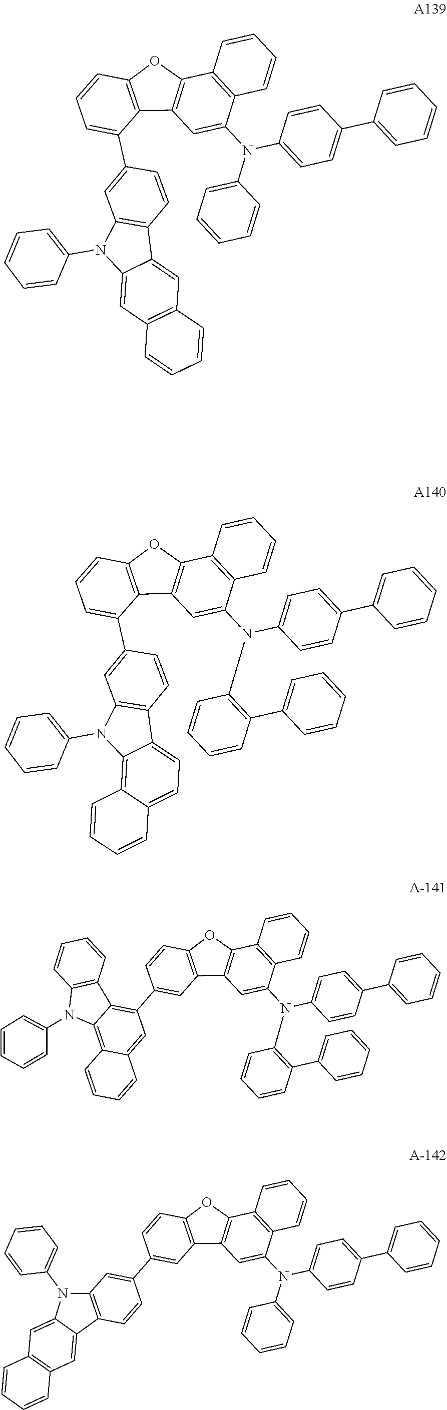 [SMILES: c1ccc(-c2ccc(N(c3ccccc3)c3cc4c(oc5cccc(-c6ccc7c8cc9ccccc9cc8n(-c8ccccc8)c7c6)c54)c4ccccc34)cc2)cc1 ; c1ccc(-c2ccc(N(c3ccccc3-c3ccccc3)c3cc4c(oc5cccc(-c6ccc7c8ccc9ccccc9c8n(-c8ccccc8)c7c6)c54)c4ccccc34)cc2)cc1 ; c1ccc(-c2ccc(N(c3ccccc3-c3ccccc3)c3cc4c5cc(-c6cc7ccccc7c7c6c6ccccc6n7-c6ccccc6)ccc5oc4c4ccccc34)cc2)cc1 ; c1ccc(-c2ccc(N(c3ccccc3)c3cc4c5cc(-c6ccc7c8cc9ccccc9cc8n(-c8ccccc8)c7c6)ccc5oc4c4ccccc34)cc2)cc1]

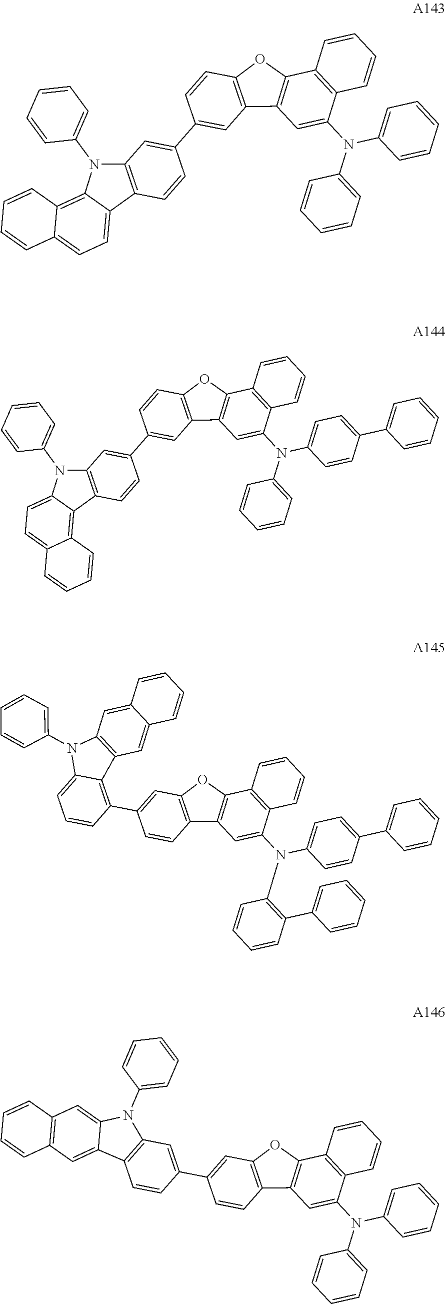 [SMILES: c1ccc(N(c2ccccc2)c2cc3c4cc(-c5ccc6c7ccc8ccccc8c7n(-c7ccccc7)c6c5)ccc4oc3c3ccccc23)cc1 ; c1ccc(-c2ccc(N(c3ccccc3)c3cc4c5cc(-c6ccc7c8c9ccccc9ccc8n(-c8ccccc8)c7c6)ccc5oc4c4ccccc34)cc2)cc1 ; c1ccc(-c2ccc(N(c3ccccc3-c3ccccc3)c3cc4c5ccc(-c6cccc7c6c6cc8ccccc8cc6n7-c6ccccc6)cc5oc4c4ccccc34)cc2)cc1 ; c1ccc(N(c2ccccc2)c2cc3c4ccc(-c5ccc6c7cc8ccccc8cc7n(-c7ccccc7)c6c5)cc4oc3c3ccccc23)cc1]

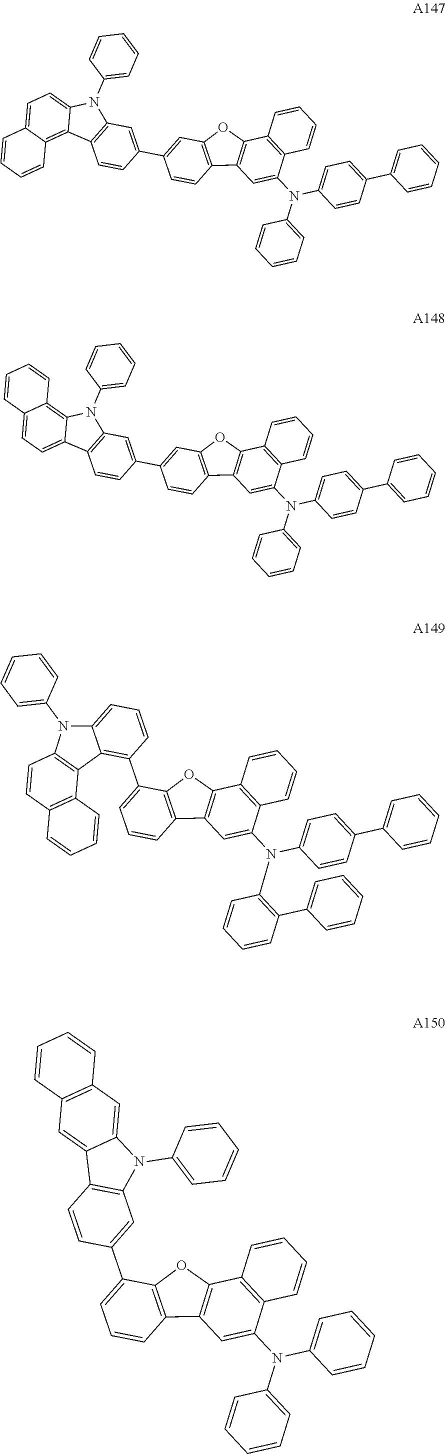 [SMILES: c1ccc(-c2ccc(N(c3ccccc3)c3cc4c5ccc(-c6ccc7c8c9ccccc9ccc8n(-c8ccccc8)c7c6)cc5oc4c4ccccc34)cc2)cc1 ; c1ccc(-c2ccc(N(c3ccccc3)c3cc4c5ccc(-c6ccc7c8ccc9ccccc9c8n(-c8ccccc8)c7c6)cc5oc4c4ccccc34)cc2)cc1 ; c1ccc(-c2ccc(N(c3ccccc3-c3ccccc3)c3cc4c5cccc(-c6cccc7c6c6c8ccccc8ccc6n7-c6ccccc6)c5oc4c4ccccc34)cc2)cc1 ; c1ccc(N(c2ccccc2)c2cc3c4cccc(-c5ccc6c7cc8ccccc8cc7n(-c7ccccc7)c6c5)c4oc3c3ccccc23)cc1]

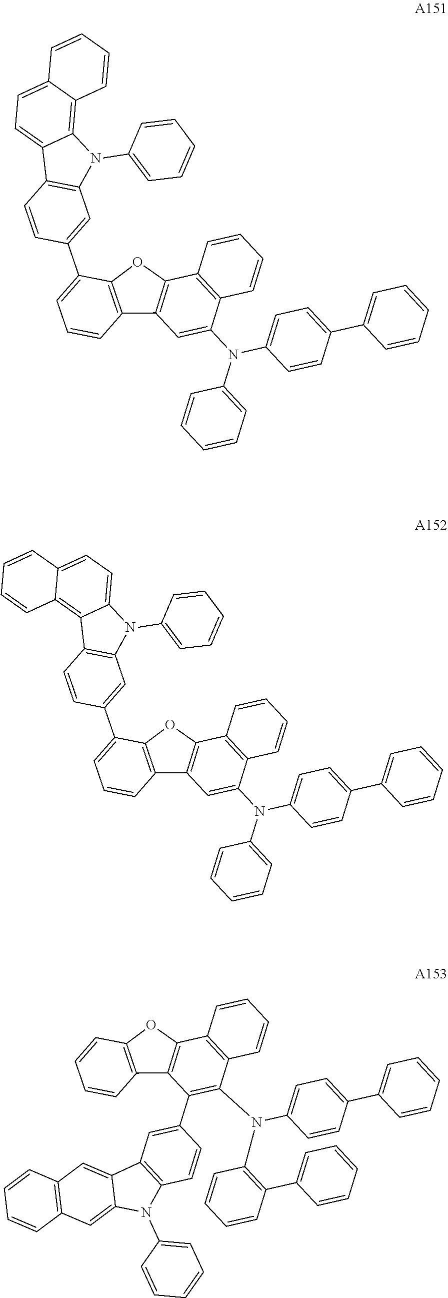 [SMILES: c1ccc(-c2ccc(N(c3ccccc3)c3cc4c5cccc(-c6ccc7c8ccc9ccccc9c8n(-c8ccccc8)c7c6)c5oc4c4ccccc34)cc2)cc1 ; c1ccc(-c2ccc(N(c3ccccc3)c3cc4c5cccc(-c6ccc7c8c9ccccc9ccc8n(-c8ccccc8)c7c6)c5oc4c4ccccc34)cc2)cc1 ; c1ccc(-c2ccc(N(c3ccccc3-c3ccccc3)c3c(-c4ccc5c(c4)c4cc6ccccc6cc4n5-c4ccccc4)c4c5ccccc5oc4c4ccccc34)cc2)cc1]

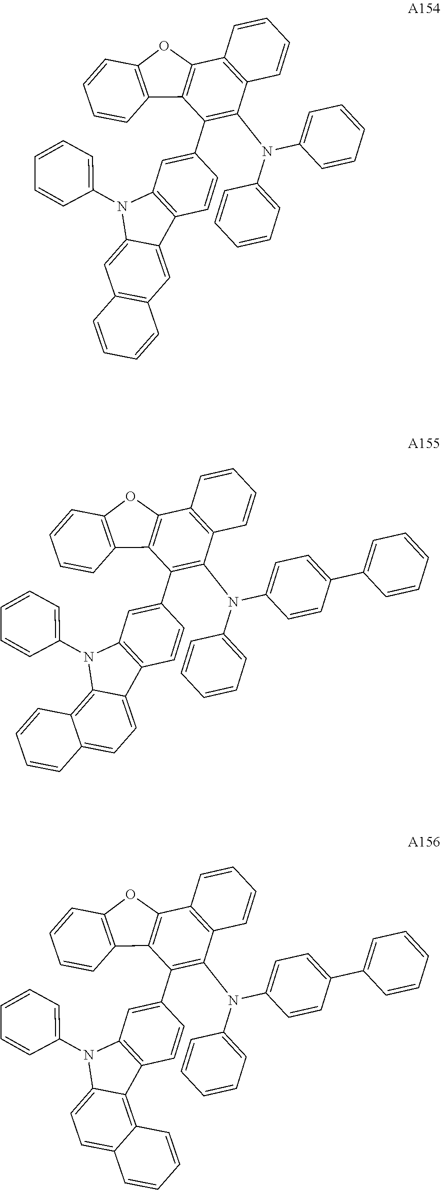 [SMILES: c1ccc(N(c2ccccc2)c2c(-c3ccc4c5cc6ccccc6cc5n(-c5ccccc5)c4c3)c3c4ccccc4oc3c3ccccc23)cc1 ; c1ccc(-c2ccc(N(c3ccccc3)c3c(-c4ccc5c6ccc7ccccc7c6n(-c6ccccc6)c5c4)c4c5ccccc5oc4c4ccccc34)cc2)cc1 ; c1ccc(-c2ccc(N(c3ccccc3)c3c(-c4ccc5c6c7ccccc7ccc6n(-c6ccccc6)c5c4)c4c5ccccc5oc4c4ccccc34)cc2)cc1]

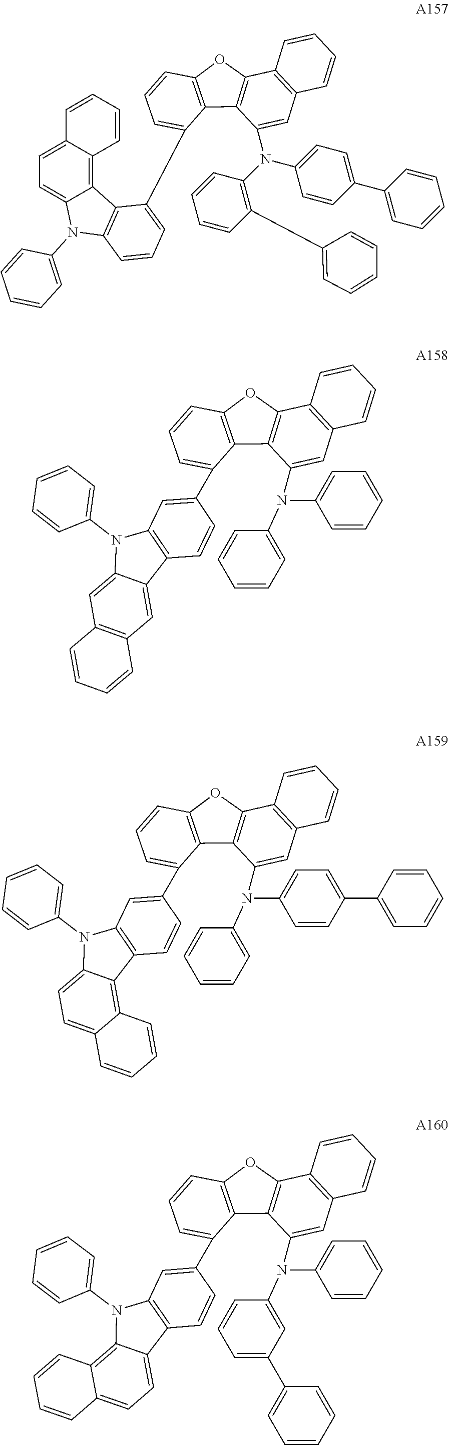 [SMILES: c1ccc(-c2ccc(N(c3ccccc3-c3ccccc3)c3cc4ccccc4c4oc5cccc(-c6cccc7c6c6c8ccccc8ccc6n7-c6ccccc6)c5c34)cc2)cc1 ; c1ccc(N(c2ccccc2)c2cc3ccccc3c3oc4cccc(-c5ccc6c7cc8ccccc8cc7n(-c7ccccc7)c6c5)c4c23)cc1 ; c1ccc(-c2ccc(N(c3ccccc3)c3cc4ccccc4c4oc5cccc(-c6ccc7c8c9ccccc9ccc8n(-c8ccccc8)c7c6)c5c34)cc2)cc1 ; c1ccc(-c2cccc(N(c3ccccc3)c3cc4ccccc4c4oc5cccc(-c6ccc7c8ccc9ccccc9c8n(-c8ccccc8)c7c6)c5c34)c2)cc1]

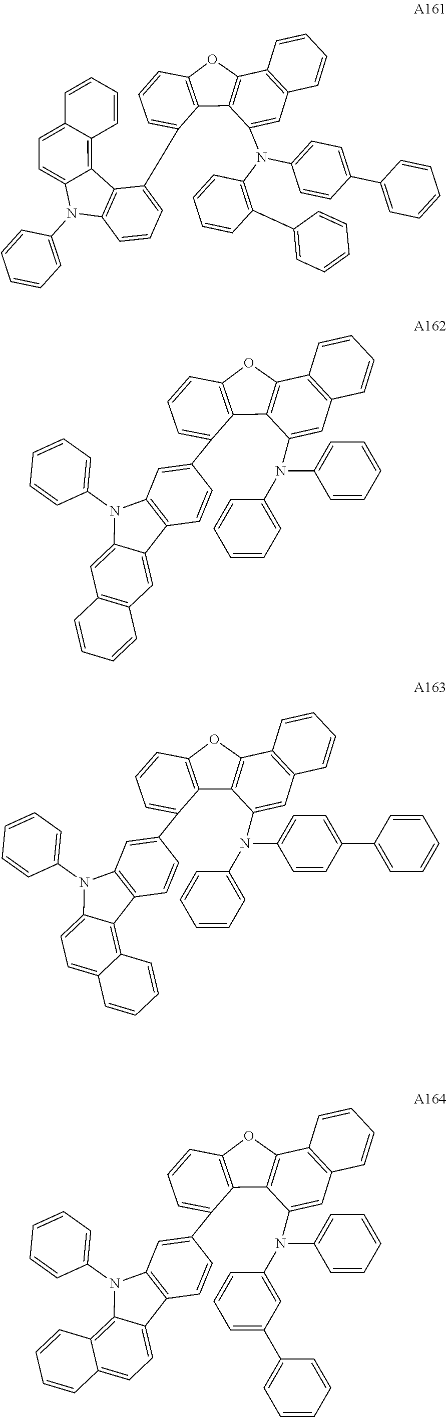 [SMILES: c1ccc(-c2ccc(N(c3ccccc3-c3ccccc3)c3cc4ccccc4c4oc5cccc(-c6cccc7c6c6c8ccccc8ccc6n7-c6ccccc6)c5c34)cc2)cc1 ; c1ccc(N(c2ccccc2)c2cc3ccccc3c3oc4cccc(-c5ccc6c7cc8ccccc8cc7n(-c7ccccc7)c6c5)c4c23)cc1 ; c1ccc(-c2ccc(N(c3ccccc3)c3cc4ccccc4c4oc5cccc(-c6ccc7c8c9ccccc9ccc8n(-c8ccccc8)c7c6)c5c34)cc2)cc1 ; c1ccc(-c2cccc(N(c3ccccc3)c3cc4ccccc4c4oc5cccc(-c6ccc7c8ccc9ccccc9c8n(-c8ccccc8)c7c6)c5c34)c2)cc1]

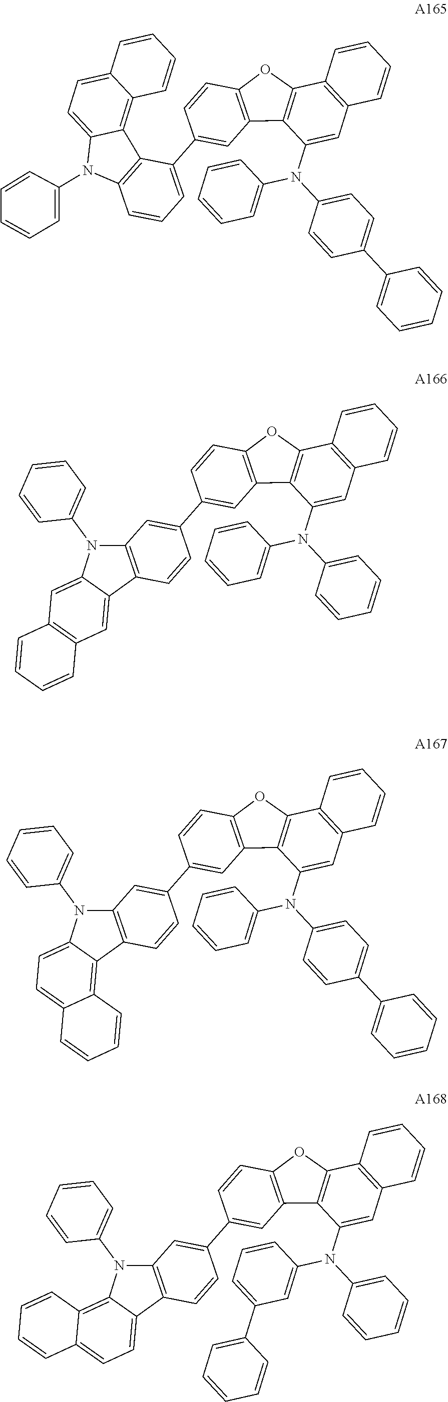 [SMILES: c1ccc(-c2ccc(N(c3ccccc3)c3cc4ccccc4c4oc5ccc(-c6cccc7c6c6c8ccccc8ccc6n7-c6ccccc6)cc5c34)cc2)cc1 ; c1ccc(N(c2ccccc2)c2cc3ccccc3c3oc4ccc(-c5ccc6c7cc8ccccc8cc7n(-c7ccccc7)c6c5)cc4c23)cc1 ; c1ccc(-c2ccc(N(c3ccccc3)c3cc4ccccc4c4oc5ccc(-c6ccc7c8c9ccccc9ccc8n(-c8ccccc8)c7c6)cc5c34)cc2)cc1 ; c1ccc(-c2cccc(N(c3ccccc3)c3cc4ccccc4c4oc5ccc(-c6ccc7c8ccc9ccccc9c8n(-c8ccccc8)c7c6)cc5c34)c2)cc1]

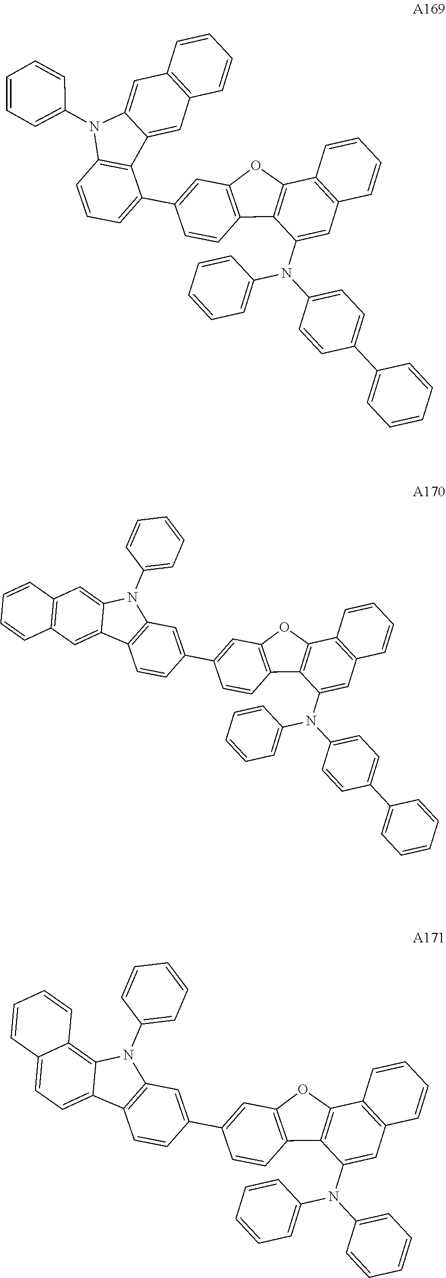 [SMILES: c1ccc(-c2ccc(N(c3ccccc3)c3cc4ccccc4c4oc5cc(-c6cccc7c6c6cc8ccccc8cc6n7-c6ccccc6)ccc5c34)cc2)cc1 ; c1ccc(-c2ccc(N(c3ccccc3)c3cc4ccccc4c4oc5cc(-c6ccc7c8cc9ccccc9cc8n(-c8ccccc8)c7c6)ccc5c34)cc2)cc1 ; c1ccc(N(c2ccccc2)c2cc3ccccc3c3oc4cc(-c5ccc6c7ccc8ccccc8c7n(-c7ccccc7)c6c5)ccc4c23)cc1]

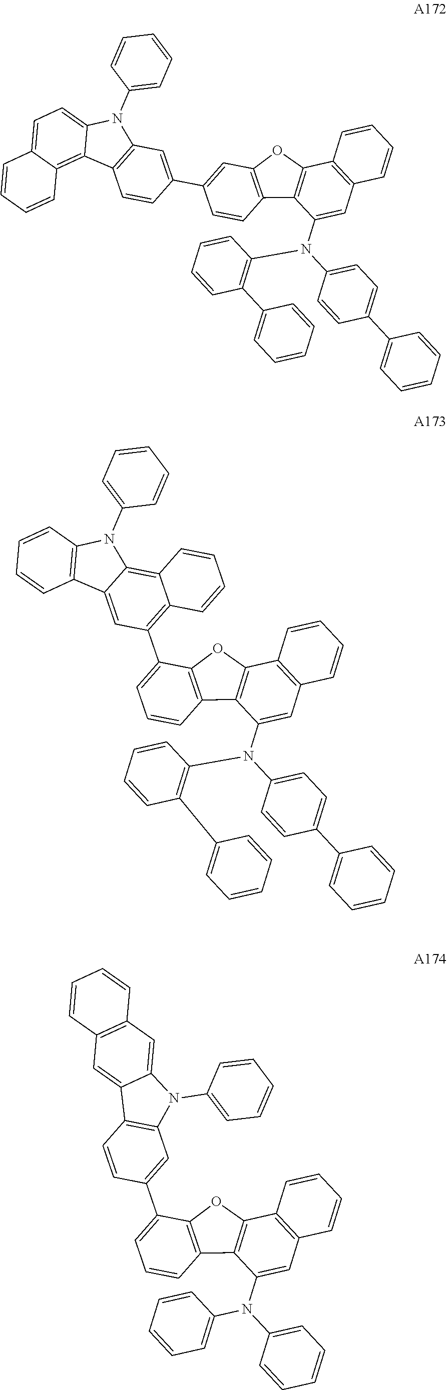 [SMILES: c1ccc(-c2ccc(N(c3ccccc3-c3ccccc3)c3cc4ccccc4c4oc5cc(-c6ccc7c8c9ccccc9ccc8n(-c8ccccc8)c7c6)ccc5c34)cc2)cc1 ; c1ccc(-c2ccc(N(c3ccccc3-c3ccccc3)c3cc4ccccc4c4oc5c(-c6cc7c8ccccc8n(-c8ccccc8)c7c7ccccc67)cccc5c34)cc2)cc1 ; c1ccc(N(c2ccccc2)c2cc3ccccc3c3oc4c(-c5ccc6c7cc8ccccc8cc7n(-c7ccccc7)c6c5)cccc4c23)cc1]

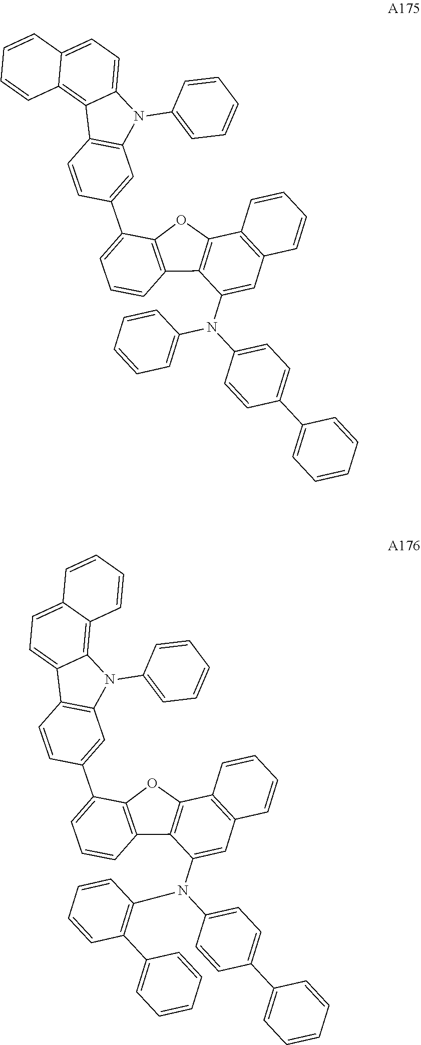 [SMILES: c1ccc(-c2ccc(N(c3ccccc3)c3cc4ccccc4c4oc5c(-c6ccc7c8c9ccccc9ccc8n(-c8ccccc8)c7c6)cccc5c34)cc2)cc1 ; c1ccc(-c2ccc(N(c3ccccc3-c3ccccc3)c3cc4ccccc4c4oc5c(-c6ccc7c8ccc9ccccc9c8n(-c8ccccc8)c7c6)cccc5c34)cc2)cc1]

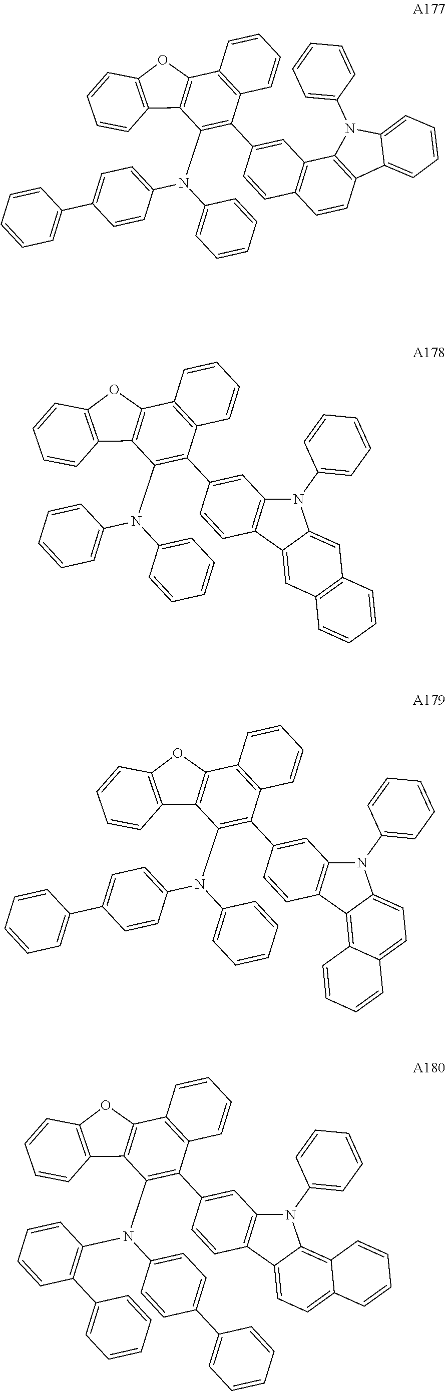 [SMILES: c1ccc(-c2ccc(N(c3ccccc3)c3c(-c4ccc5ccc6c7ccccc7n(-c7ccccc7)c6c5c4)c4ccccc4c4oc5ccccc5c34)cc2)cc1 ; c1ccc(N(c2ccccc2)c2c(-c3ccc4c5cc6ccccc6cc5n(-c5ccccc5)c4c3)c3ccccc3c3oc4ccccc4c23)cc1 ; c1ccc(-c2ccc(N(c3ccccc3)c3c(-c4ccc5c6c7ccccc7ccc6n(-c6ccccc6)c5c4)c4ccccc4c4oc5ccccc5c34)cc2)cc1 ; c1ccc(-c2ccc(N(c3ccccc3-c3ccccc3)c3c(-c4ccc5c6ccc7ccccc7c6n(-c6ccccc6)c5c4)c4ccccc4c4oc5ccccc5c34)cc2)cc1]

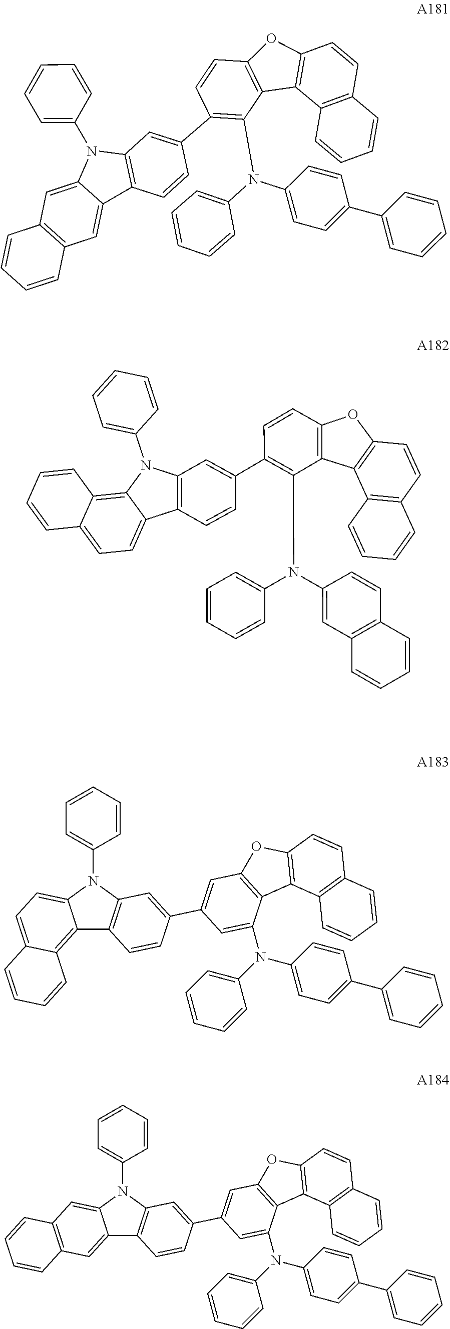 [SMILES: c1ccc(-c2ccc(N(c3ccccc3)c3c(-c4ccc5c6cc7ccccc7cc6n(-c6ccccc6)c5c4)ccc4oc5ccc6ccccc6c5c34)cc2)cc1 ; c1ccc(N(c2ccc3ccccc3c2)c2c(-c3ccc4c5ccc6ccccc6c5n(-c5ccccc5)c4c3)ccc3oc4ccc5ccccc5c4c23)cc1 ; c1ccc(-c2ccc(N(c3ccccc3)c3cc(-c4ccc5c6c7ccccc7ccc6n(-c6ccccc6)c5c4)cc4oc5ccc6ccccc6c5c34)cc2)cc1 ; c1ccc(-c2ccc(N(c3ccccc3)c3cc(-c4ccc5c6cc7ccccc7cc6n(-c6ccccc6)c5c4)cc4oc5ccc6ccccc6c5c34)cc2)cc1]

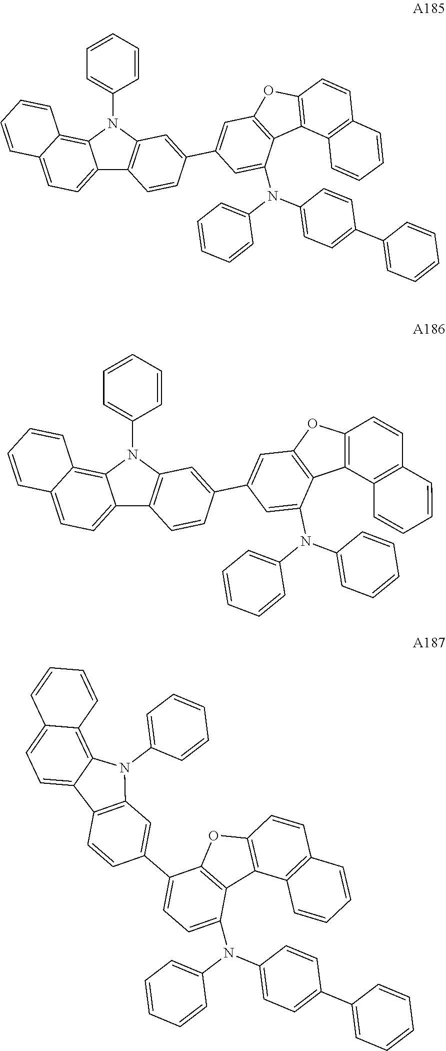 [SMILES: c1ccc(-c2ccc(N(c3ccccc3)c3cc(-c4ccc5c6ccc7ccccc7c6n(-c6ccccc6)c5c4)cc4oc5ccc6ccccc6c5c34)cc2)cc1 ; c1ccc(N(c2ccccc2)c2cc(-c3ccc4c5ccc6ccccc6c5n(-c5ccccc5)c4c3)cc3oc4ccc5ccccc5c4c23)cc1 ; c1ccc(-c2ccc(N(c3ccccc3)c3ccc(-c4ccc5c6ccc7ccccc7c6n(-c6ccccc6)c5c4)c4oc5ccc6ccccc6c5c34)cc2)cc1]

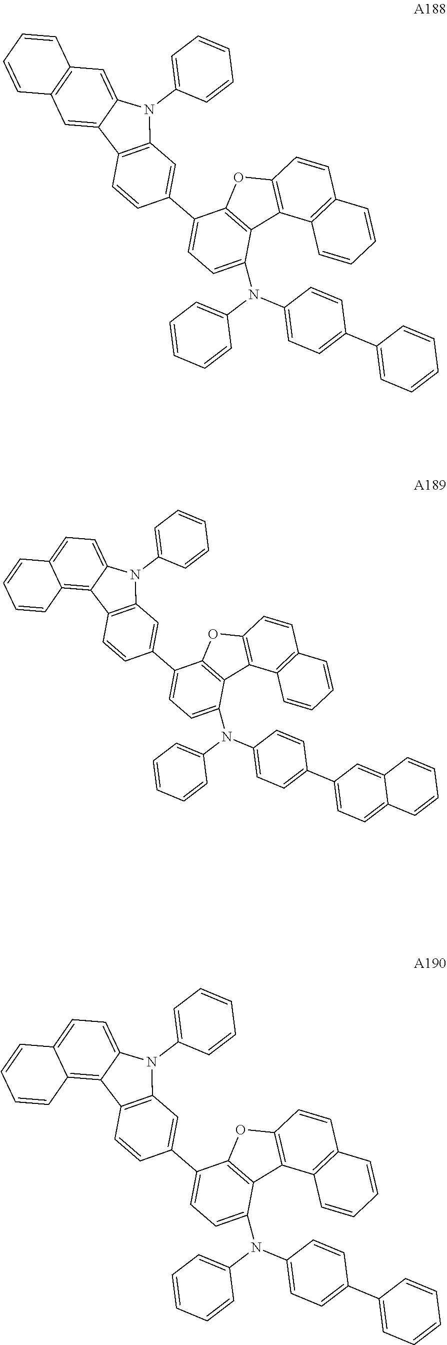 [SMILES: c1ccc(-c2ccc(N(c3ccccc3)c3ccc(-c4ccc5c6cc7ccccc7cc6n(-c6ccccc6)c5c4)c4oc5ccc6ccccc6c5c34)cc2)cc1 ; c1ccc(N(c2ccc(-c3ccc4ccccc4c3)cc2)c2ccc(-c3ccc4c5c6ccccc6ccc5n(-c5ccccc5)c4c3)c3oc4ccc5ccccc5c4c23)cc1 ; c1ccc(-c2ccc(N(c3ccccc3)c3ccc(-c4ccc5c6c7ccccc7ccc6n(-c6ccccc6)c5c4)c4oc5ccc6ccccc6c5c34)cc2)cc1]

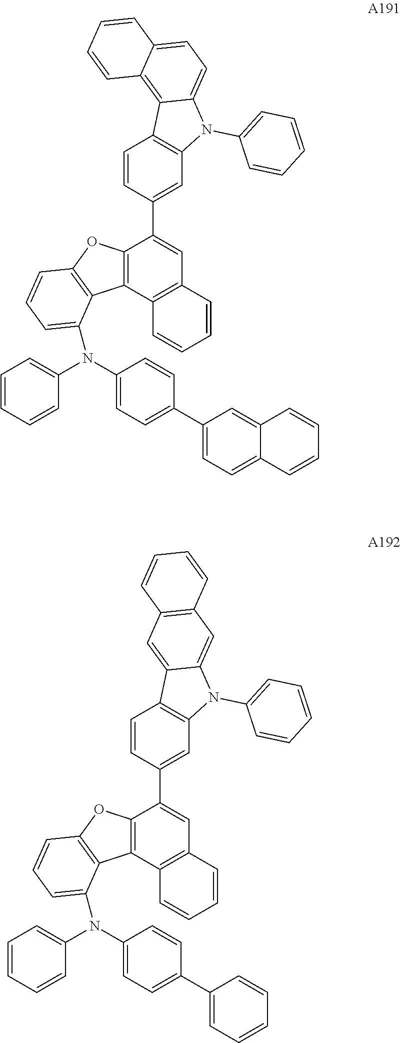 [SMILES: c1ccc(N(c2ccc(-c3ccc4ccccc4c3)cc2)c2cccc3oc4c(-c5ccc6c7c8ccccc8ccc7n(-c7ccccc7)c6c5)cc5ccccc5c4c23)cc1 ; c1ccc(-c2ccc(N(c3ccccc3)c3cccc4oc5c(-c6ccc7c8cc9ccccc9cc8n(-c8ccccc8)c7c6)cc6ccccc6c5c34)cc2)cc1]

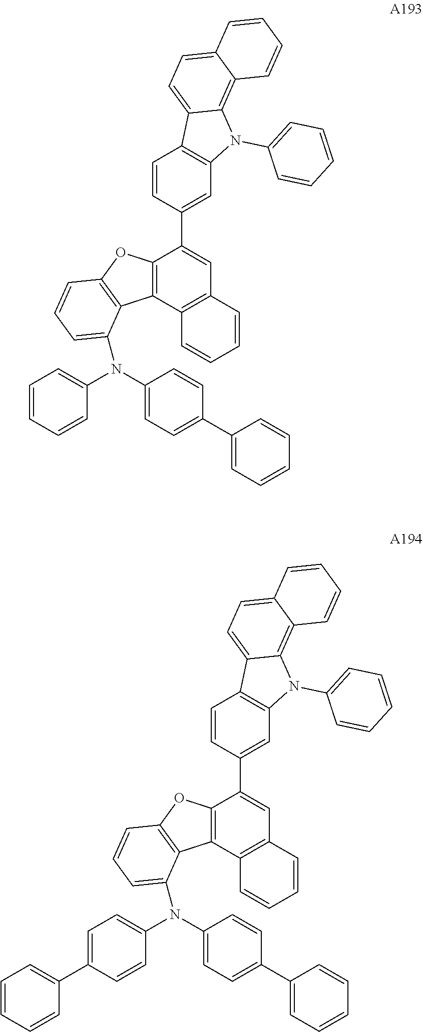 [SMILES: c1ccc(-c2ccc(N(c3ccccc3)c3cccc4oc5c(-c6ccc7c8ccc9ccccc9c8n(-c8ccccc8)c7c6)cc6ccccc6c5c34)cc2)cc1 ; c1ccc(-c2ccc(N(c3ccc(-c4ccccc4)cc3)c3cccc4oc5c(-c6ccc7c8ccc9ccccc9c8n(-c8ccccc8)c7c6)cc6ccccc6c5c34)cc2)cc1]

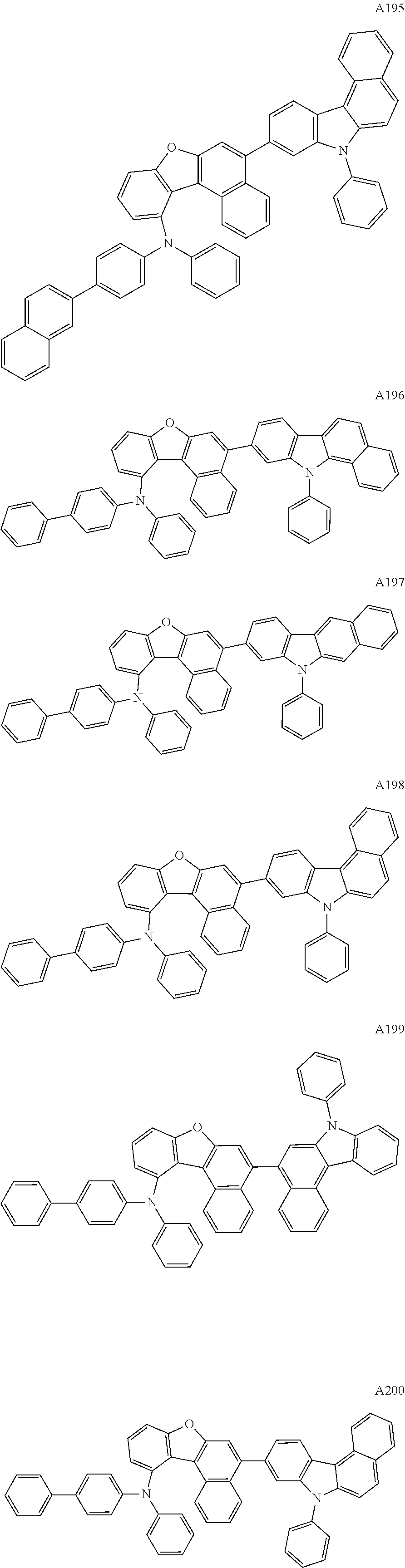

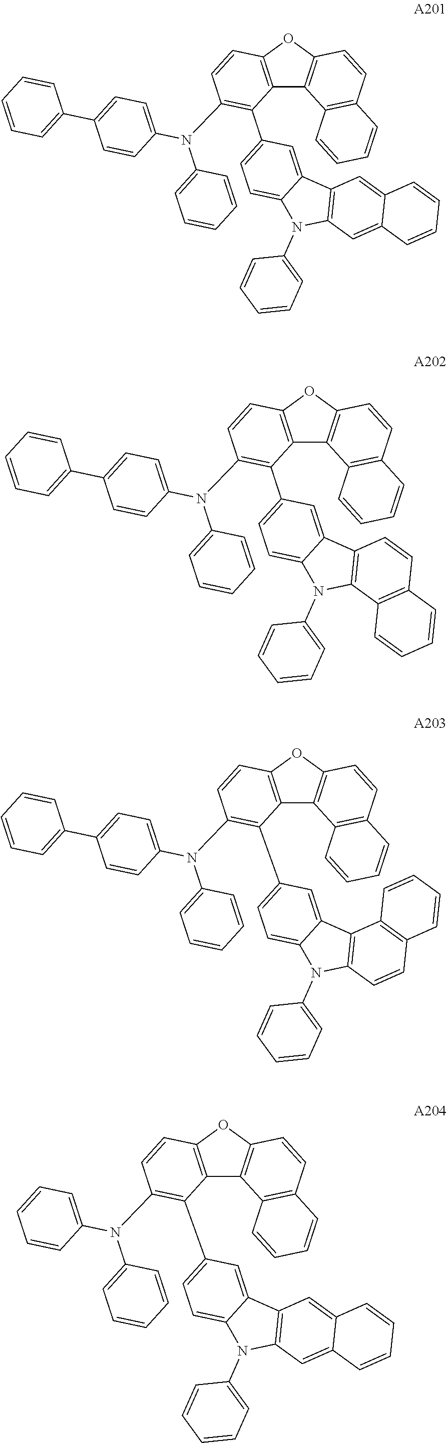 [SMILES: c1ccc(-c2ccc(N(c3ccccc3)c3ccc4oc5ccc6ccccc6c5c4c3-c3ccc4c(c3)c3cc5ccccc5cc3n4-c3ccccc3)cc2)cc1 ; c1ccc(-c2ccc(N(c3ccccc3)c3ccc4oc5ccc6ccccc6c5c4c3-c3ccc4c(c3)c3ccc5ccccc5c3n4-c3ccccc3)cc2)cc1 ; c1ccc(-c2ccc(N(c3ccccc3)c3ccc4oc5ccc6ccccc6c5c4c3-c3ccc4c(c3)c3c5ccccc5ccc3n4-c3ccccc3)cc2)cc1 ; c1ccc(N(c2ccccc2)c2ccc3oc4ccc5ccccc5c4c3c2-c2ccc3c(c2)c2cc4ccccc4cc2n3-c2ccccc2)cc1]

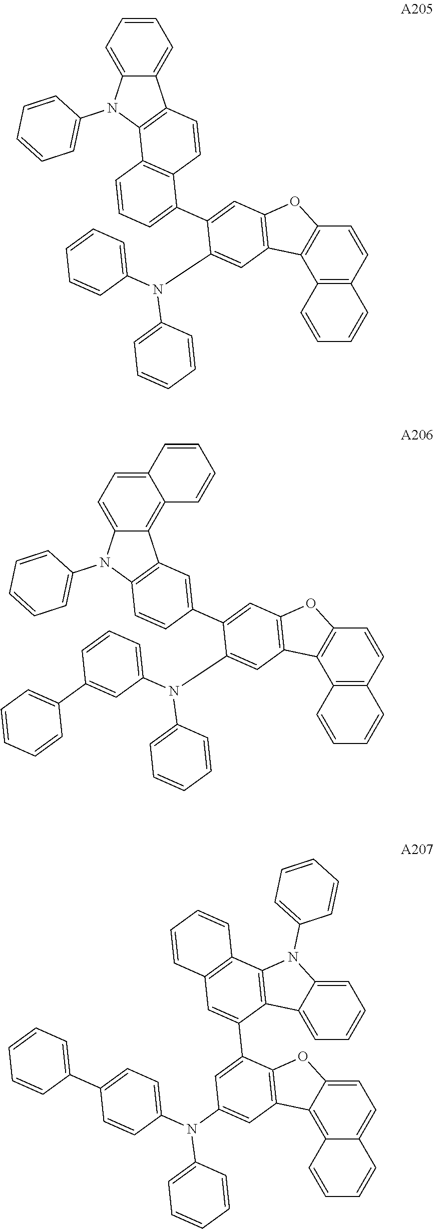 [SMILES: c1ccc(N(c2ccccc2)c2cc3c(cc2-c2cccc4c2ccc2c5ccccc5n(-c5ccccc5)c42)oc2ccc4ccccc4c23)cc1 ; c1ccc(-c2cccc(N(c3ccccc3)c3cc4c(cc3-c3ccc5c(c3)c3c6ccccc6ccc3n5-c3ccccc3)oc3ccc5ccccc5c34)c2)cc1 ; c1ccc(-c2ccc(N(c3ccccc3)c3cc(-c4cc5ccccc5c5c4c4ccccc4n5-c4ccccc4)c4oc5ccc6ccccc6c5c4c3)cc2)cc1]

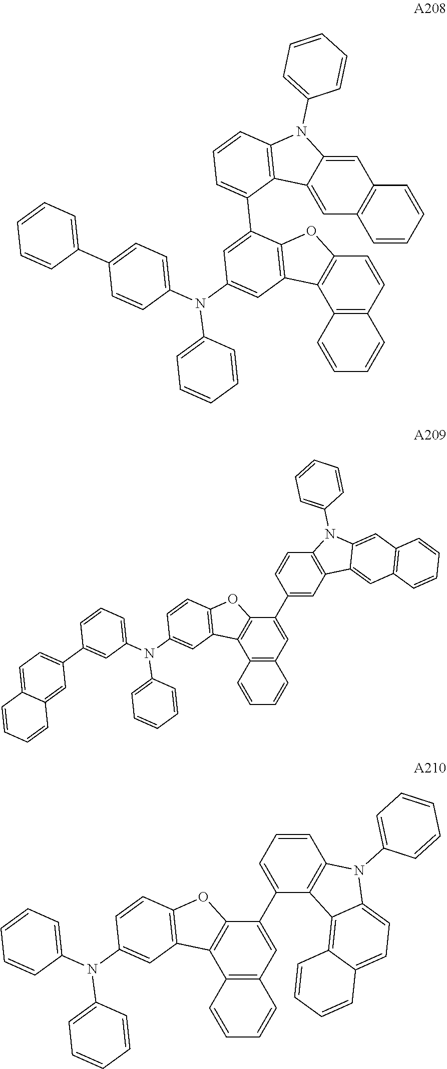 [SMILES: c1ccc(-c2ccc(N(c3ccccc3)c3cc(-c4cccc5c4c4cc6ccccc6cc4n5-c4ccccc4)c4oc5ccc6ccccc6c5c4c3)cc2)cc1 ; c1ccc(N(c2cccc(-c3ccc4ccccc4c3)c2)c2ccc3oc4c(-c5ccc6c(c5)c5cc7ccccc7cc5n6-c5ccccc5)cc5ccccc5c4c3c2)cc1 ; c1ccc(N(c2ccccc2)c2ccc3oc4c(-c5cccc6c5c5c7ccccc7ccc5n6-c5ccccc5)cc5ccccc5c4c3c2)cc1]

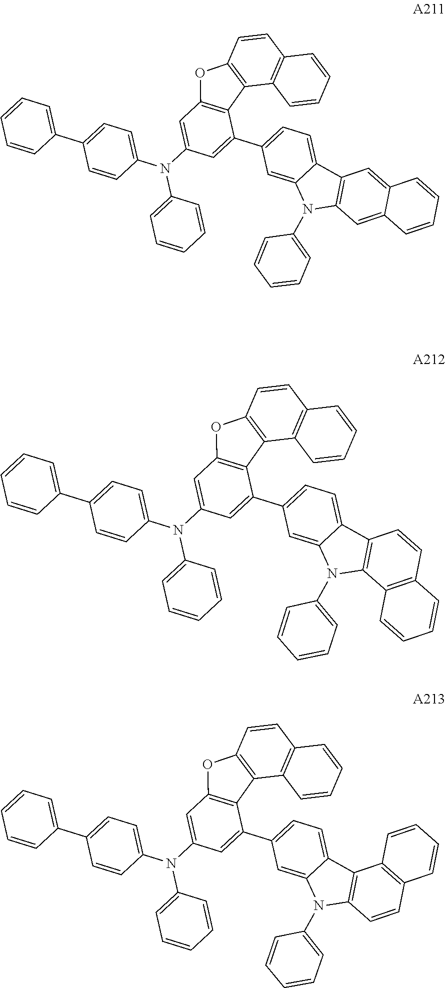 [SMILES: c1ccc(-c2ccc(N(c3ccccc3)c3cc(-c4ccc5c6cc7ccccc7cc6n(-c6ccccc6)c5c4)c4c(c3)oc3ccc5ccccc5c34)cc2)cc1 ; c1ccc(-c2ccc(N(c3ccccc3)c3cc(-c4ccc5c6ccc7ccccc7c6n(-c6ccccc6)c5c4)c4c(c3)oc3ccc5ccccc5c34)cc2)cc1 ; c1ccc(-c2ccc(N(c3ccccc3)c3cc(-c4ccc5c6c7ccccc7ccc6n(-c6ccccc6)c5c4)c4c(c3)oc3ccc5ccccc5c34)cc2)cc1]

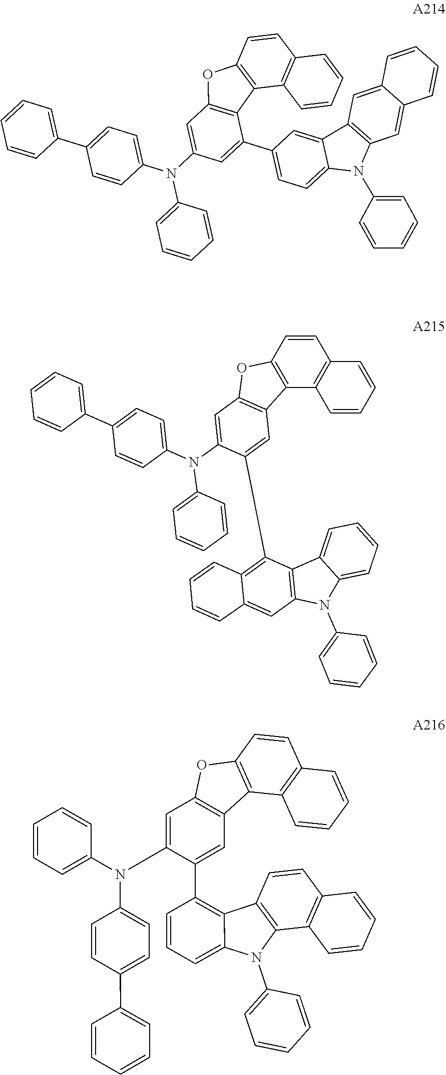 [SMILES: c1ccc(-c2ccc(N(c3ccccc3)c3cc(-c4ccc5c(c4)c4cc6ccccc6cc4n5-c4ccccc4)c4c(c3)oc3ccc5ccccc5c34)cc2)cc1 ; c1ccc(-c2ccc(N(c3ccccc3)c3cc4oc5ccc6ccccc6c5c4cc3-c3c4ccccc4cc4c3c3ccccc3n4-c3ccccc3)cc2)cc1 ; c1ccc(-c2ccc(N(c3ccccc3)c3cc4oc5ccc6ccccc6c5c4cc3-c3cccc4c3c3ccc5ccccc5c3n4-c3ccccc3)cc2)cc1]

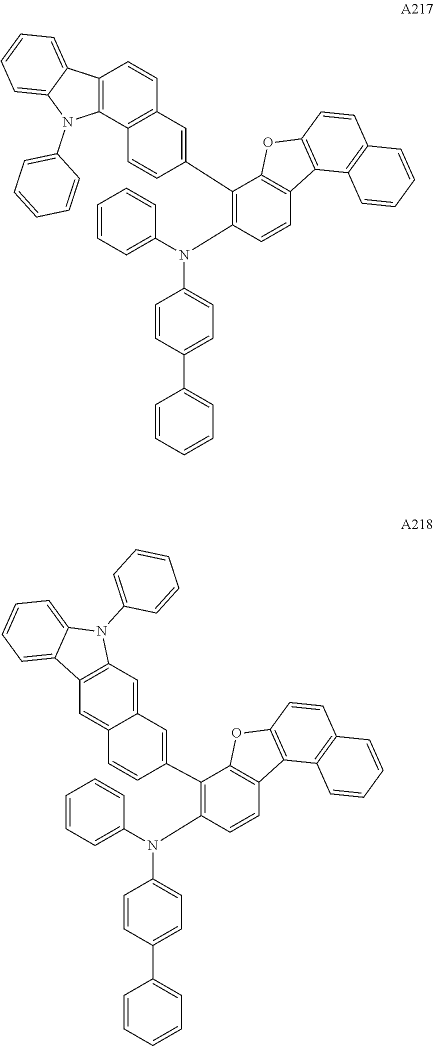 [SMILES: c1ccc(-c2ccc(N(c3ccccc3)c3ccc4c(oc5ccc6ccccc6c54)c3-c3ccc4c(ccc5c6ccccc6n(-c6ccccc6)c45)c3)cc2)cc1 ; c1ccc(-c2ccc(N(c3ccccc3)c3ccc4c(oc5ccc6ccccc6c54)c3-c3ccc4cc5c6ccccc6n(-c6ccccc6)c5cc4c3)cc2)cc1]

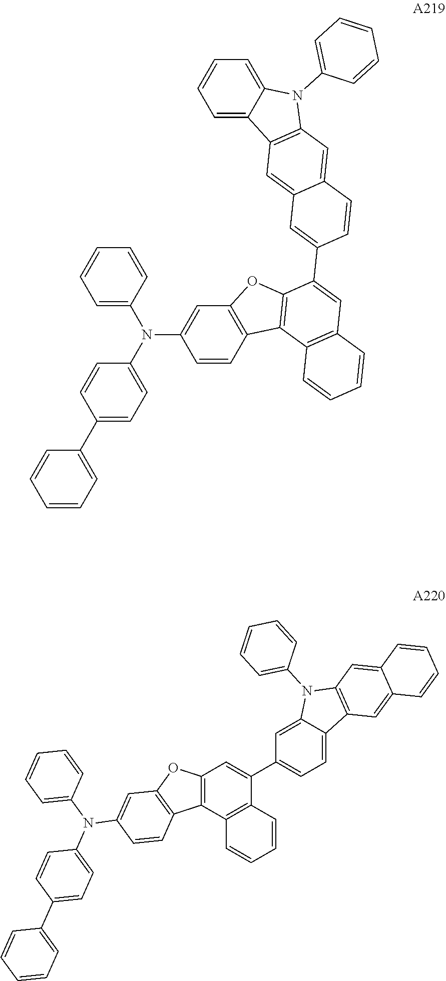 [SMILES: c1ccc(-c2ccc(N(c3ccccc3)c3ccc4c(c3)oc3c(-c5ccc6cc7c(cc6c5)c5ccccc5n7-c5ccccc5)cc5ccccc5c34)cc2)cc1 ; c1ccc(-c2ccc(N(c3ccccc3)c3ccc4c(c3)oc3cc(-c5ccc6c7cc8ccccc8cc7n(-c7ccccc7)c6c5)c5ccccc5c34)cc2)cc1]

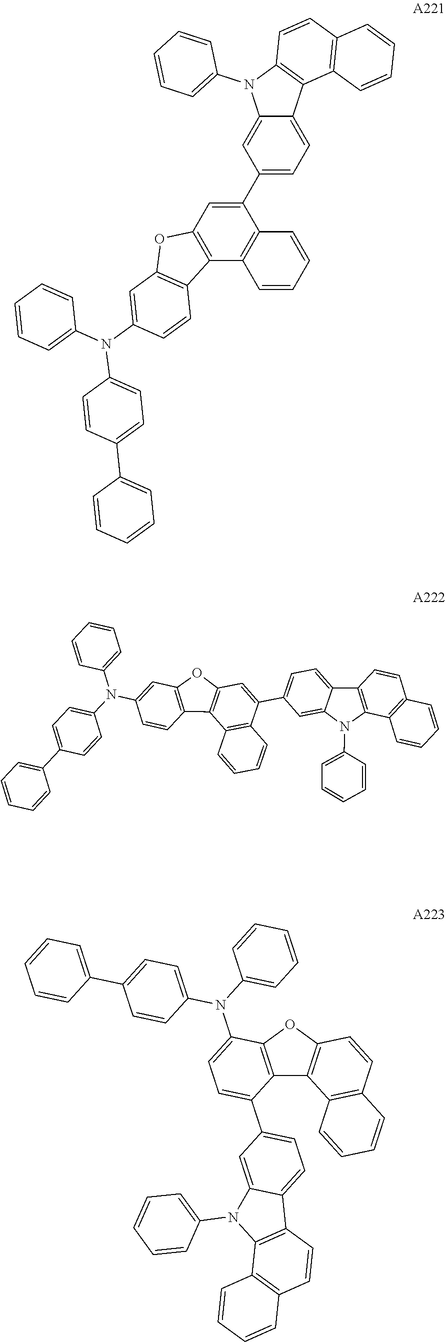 [SMILES: c1ccc(-c2ccc(N(c3ccccc3)c3ccc4c(c3)oc3cc(-c5ccc6c7c8ccccc8ccc7n(-c7ccccc7)c6c5)c5ccccc5c34)cc2)cc1 ; c1ccc(-c2ccc(N(c3ccccc3)c3ccc4c(c3)oc3cc(-c5ccc6c7ccc8ccccc8c7n(-c7ccccc7)c6c5)c5ccccc5c34)cc2)cc1 ; c1ccc(-c2ccc(N(c3ccccc3)c3ccc(-c4ccc5c6ccc7ccccc7c6n(-c6ccccc6)c5c4)c4c3oc3ccc5ccccc5c34)cc2)cc1]

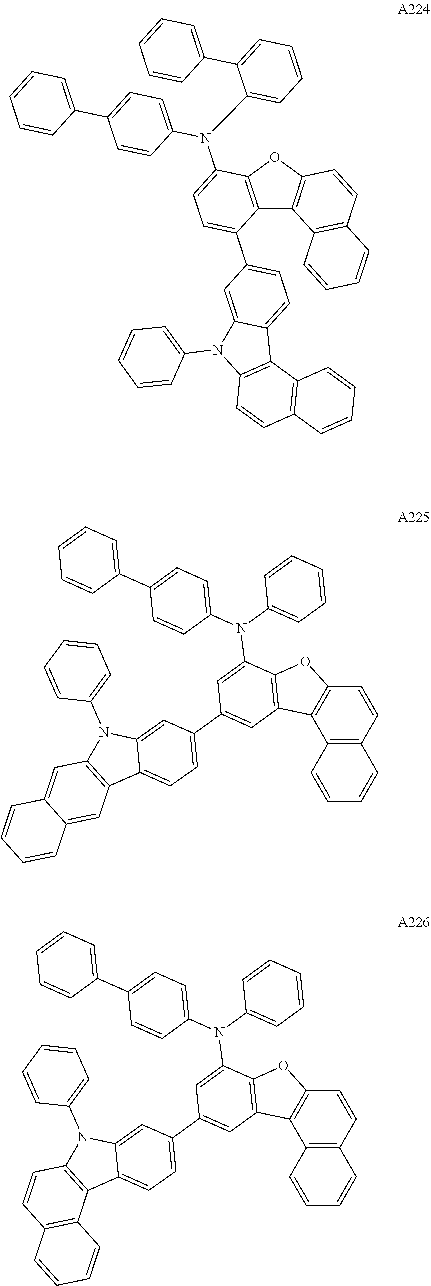 [SMILES: c1ccc(-c2ccc(N(c3ccccc3-c3ccccc3)c3ccc(-c4ccc5c6c7ccccc7ccc6n(-c6ccccc6)c5c4)c4c3oc3ccc5ccccc5c34)cc2)cc1 ; c1ccc(-c2ccc(N(c3ccccc3)c3cc(-c4ccc5c6cc7ccccc7cc6n(-c6ccccc6)c5c4)cc4c3oc3ccc5ccccc5c34)cc2)cc1 ; c1ccc(-c2ccc(N(c3ccccc3)c3cc(-c4ccc5c6c7ccccc7ccc6n(-c6ccccc6)c5c4)cc4c3oc3ccc5ccccc5c34)cc2)cc1]

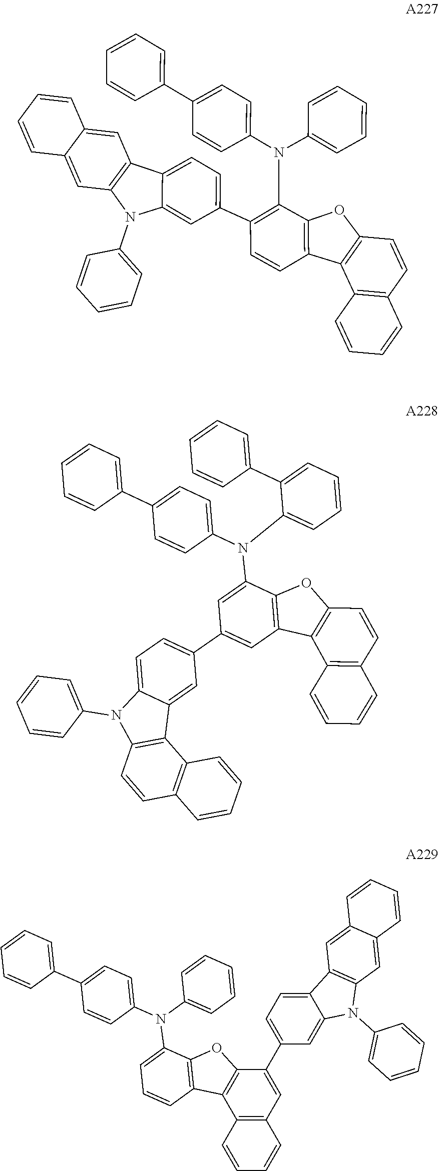 [SMILES: c1ccc(-c2ccc(N(c3ccccc3)c3c(-c4ccc5c6cc7ccccc7cc6n(-c6ccccc6)c5c4)ccc4c3oc3ccc5ccccc5c34)cc2)cc1 ; c1ccc(-c2ccc(N(c3ccccc3-c3ccccc3)c3cc(-c4ccc5c(c4)c4c6ccccc6ccc4n5-c4ccccc4)cc4c3oc3ccc5ccccc5c34)cc2)cc1 ; c1ccc(-c2ccc(N(c3ccccc3)c3cccc4c3oc3c(-c5ccc6c7cc8ccccc8cc7n(-c7ccccc7)c6c5)cc5ccccc5c34)cc2)cc1]

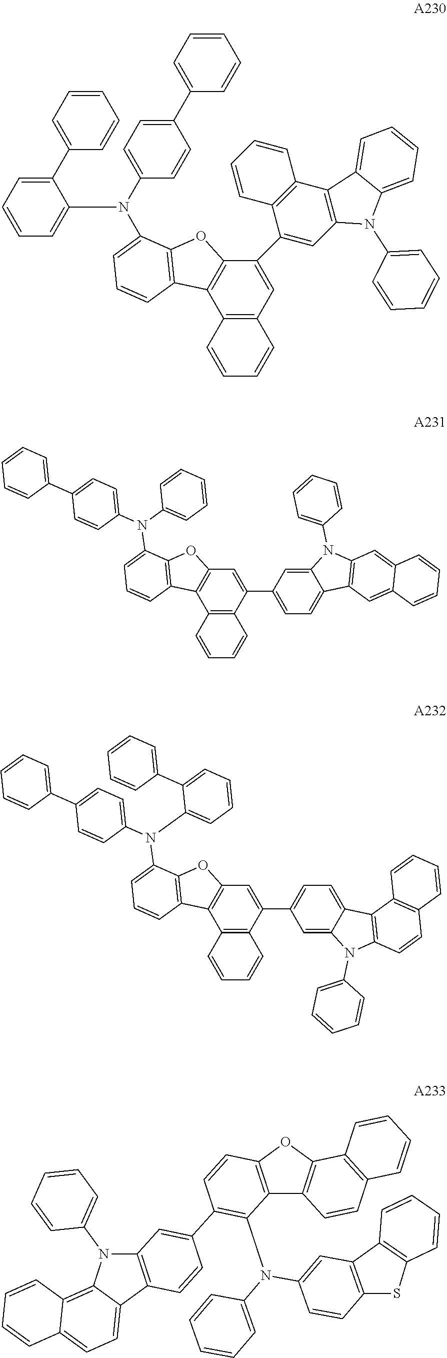 [SMILES: c1ccc(-c2ccc(N(c3ccccc3-c3ccccc3)c3cccc4c3oc3c(-c5cc6c(c7ccccc57)c5ccccc5n6-c5ccccc5)cc5ccccc5c34)cc2)cc1 ; c1ccc(-c2ccc(N(c3ccccc3)c3cccc4c3oc3cc(-c5ccc6c7cc8ccccc8cc7n(-c7ccccc7)c6c5)c5ccccc5c34)cc2)cc1 ; c1ccc(-c2ccc(N(c3ccccc3-c3ccccc3)c3cccc4c3oc3cc(-c5ccc6c7c8ccccc8ccc7n(-c7ccccc7)c6c5)c5ccccc5c34)cc2)cc1 ; c1ccc(N(c2ccc3sc4ccccc4c3c2)c2c(-c3ccc4c5ccc6ccccc6c5n(-c5ccccc5)c4c3)ccc3oc4c5ccccc5ccc4c23)cc1]

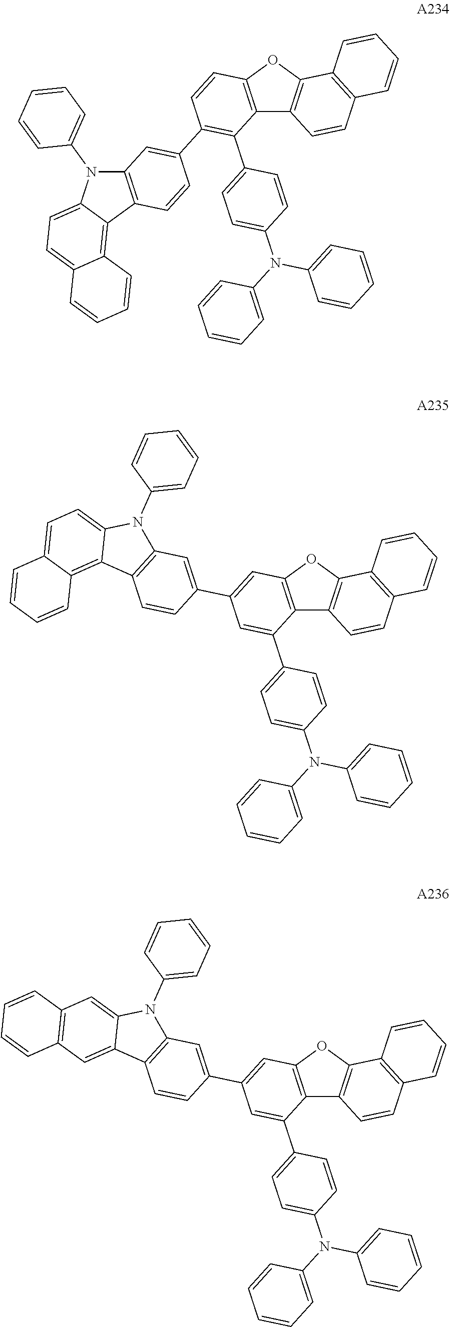 [SMILES: c1ccc(N(c2ccccc2)c2ccc(-c3c(-c4ccc5c6c7ccccc7ccc6n(-c6ccccc6)c5c4)ccc4oc5c6ccccc6ccc5c34)cc2)cc1 ; c1ccc(N(c2ccccc2)c2ccc(-c3cc(-c4ccc5c6c7ccccc7ccc6n(-c6ccccc6)c5c4)cc4oc5c6ccccc6ccc5c34)cc2)cc1 ; c1ccc(N(c2ccccc2)c2ccc(-c3cc(-c4ccc5c6cc7ccccc7cc6n(-c6ccccc6)c5c4)cc4oc5c6ccccc6ccc5c34)cc2)cc1]

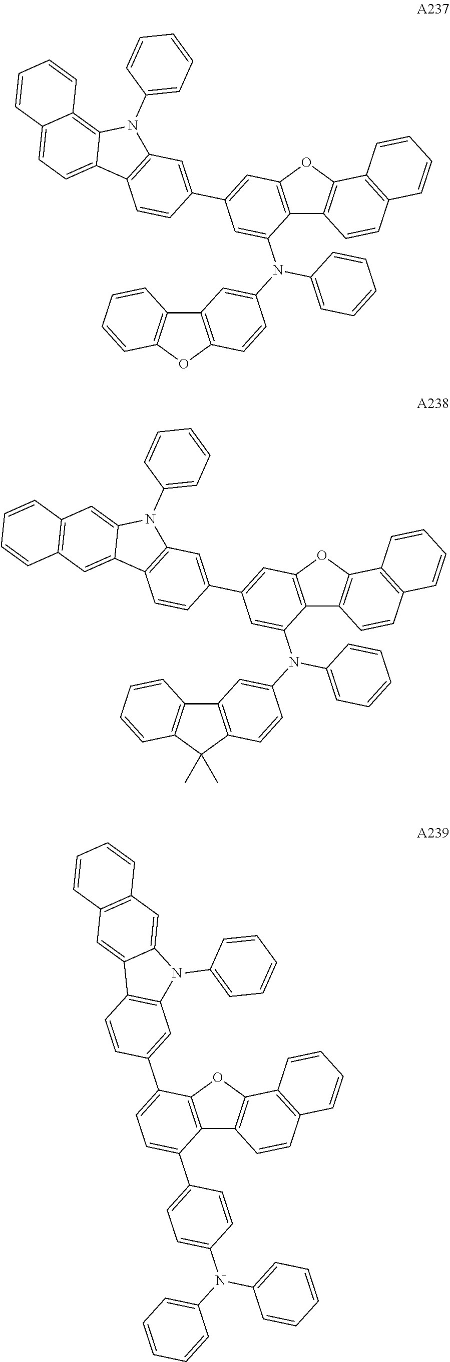 [SMILES: c1ccc(N(c2ccc3oc4ccccc4c3c2)c2cc(-c3ccc4c5ccc6ccccc6c5n(-c5ccccc5)c4c3)cc3oc4c5ccccc5ccc4c23)cc1 ; CC1(C)c2ccccc2-c2cc(N(c3ccccc3)c3cc(-c4ccc5c6cc7ccccc7cc6n(-c6ccccc6)c5c4)cc4oc5c6ccccc6ccc5c34)ccc21 ; c1ccc(N(c2ccccc2)c2ccc(-c3ccc(-c4ccc5c6cc7ccccc7cc6n(-c6ccccc6)c5c4)c4oc5c6ccccc6ccc5c34)cc2)cc1]

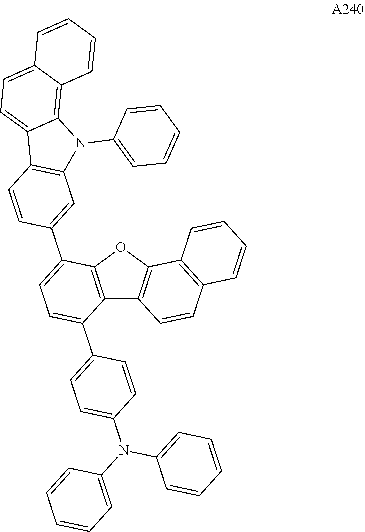 [SMILES: c1ccc(N(c2ccccc2)c2ccc(-c3ccc(-c4ccc5c6ccc7ccccc7c6n(-c6ccccc6)c5c4)c4oc5c6ccccc6ccc5c34)cc2)cc1]

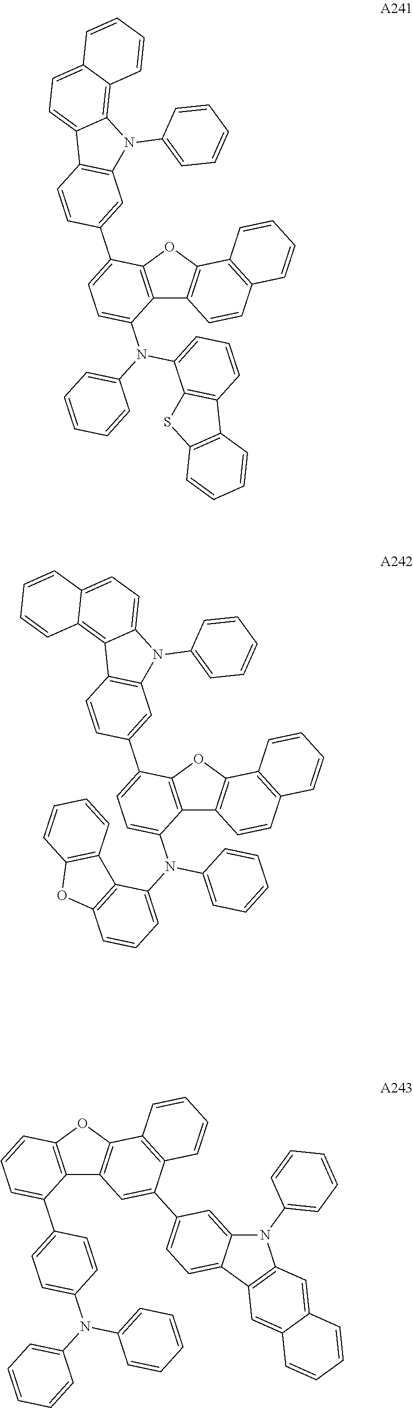 [SMILES: c1ccc(N(c2cccc3c2sc2ccccc23)c2ccc(-c3ccc4c5ccc6ccccc6c5n(-c5ccccc5)c4c3)c3oc4c5ccccc5ccc4c23)cc1 ; c1ccc(N(c2cccc3oc4ccccc4c23)c2ccc(-c3ccc4c5c6ccccc6ccc5n(-c5ccccc5)c4c3)c3oc4c5ccccc5ccc4c23)cc1 ; c1ccc(N(c2ccccc2)c2ccc(-c3cccc4oc5c6ccccc6c(-c6ccc7c8cc9ccccc9cc8n(-c8ccccc8)c7c6)cc5c34)cc2)cc1]

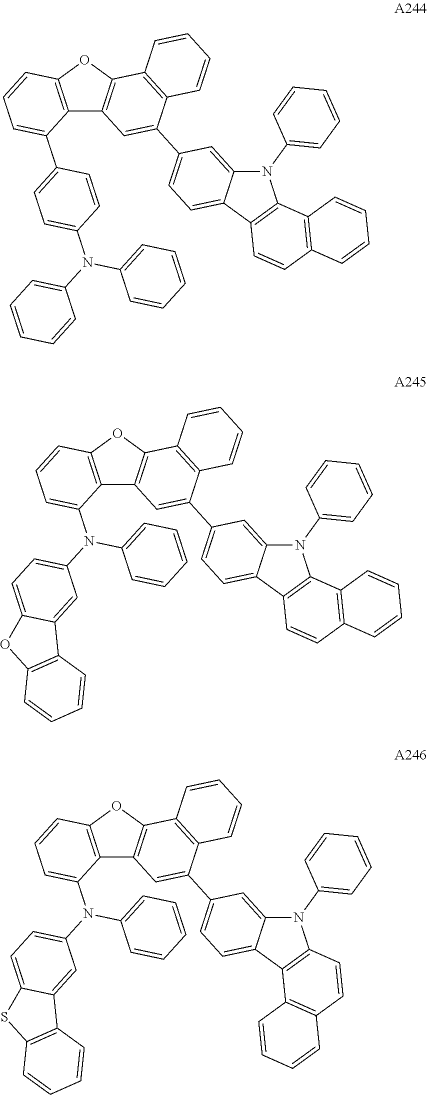 [SMILES: c1ccc(N(c2ccccc2)c2ccc(-c3cccc4oc5c6ccccc6c(-c6ccc7c8ccc9ccccc9c8n(-c8ccccc8)c7c6)cc5c34)cc2)cc1 ; c1ccc(N(c2ccc3oc4ccccc4c3c2)c2cccc3oc4c5ccccc5c(-c5ccc6c7ccc8ccccc8c7n(-c7ccccc7)c6c5)cc4c23)cc1 ; c1ccc(N(c2ccc3sc4ccccc4c3c2)c2cccc3oc4c5ccccc5c(-c5ccc6c7c8ccccc8ccc7n(-c7ccccc7)c6c5)cc4c23)cc1]

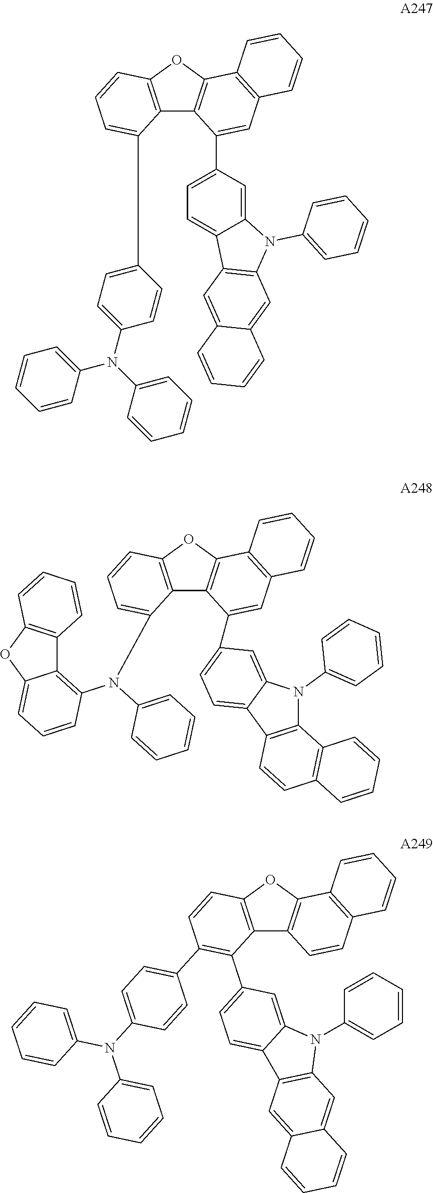 [SMILES: c1ccc(N(c2ccccc2)c2ccc(-c3cccc4oc5c6ccccc6cc(-c6ccc7c8cc9ccccc9cc8n(-c8ccccc8)c7c6)c5c34)cc2)cc1 ; c1ccc(N(c2cccc3oc4ccccc4c23)c2cccc3oc4c5ccccc5cc(-c5ccc6c7ccc8ccccc8c7n(-c7ccccc7)c6c5)c4c23)cc1 ; c1ccc(N(c2ccccc2)c2ccc(-c3ccc4oc5c6ccccc6ccc5c4c3-c3ccc4c5cc6ccccc6cc5n(-c5ccccc5)c4c3)cc2)cc1]

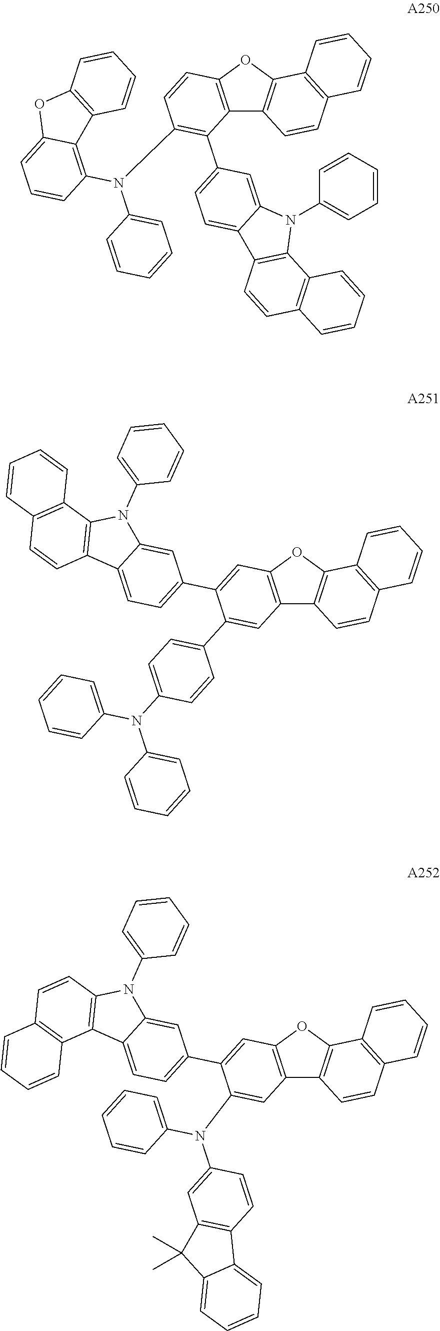 [SMILES: c1ccc(N(c2ccc3oc4c5ccccc5ccc4c3c2-c2ccc3c4ccc5ccccc5c4n(-c4ccccc4)c3c2)c2cccc3oc4ccccc4c23)cc1 ; c1ccc(N(c2ccccc2)c2ccc(-c3cc4c(cc3-c3ccc5c6ccc7ccccc7c6n(-c6ccccc6)c5c3)oc3c5ccccc5ccc43)cc2)cc1 ; CC1(C)c2ccccc2-c2ccc(N(c3ccccc3)c3cc4c(cc3-c3ccc5c6c7ccccc7ccc6n(-c6ccccc6)c5c3)oc3c5ccccc5ccc43)cc21]

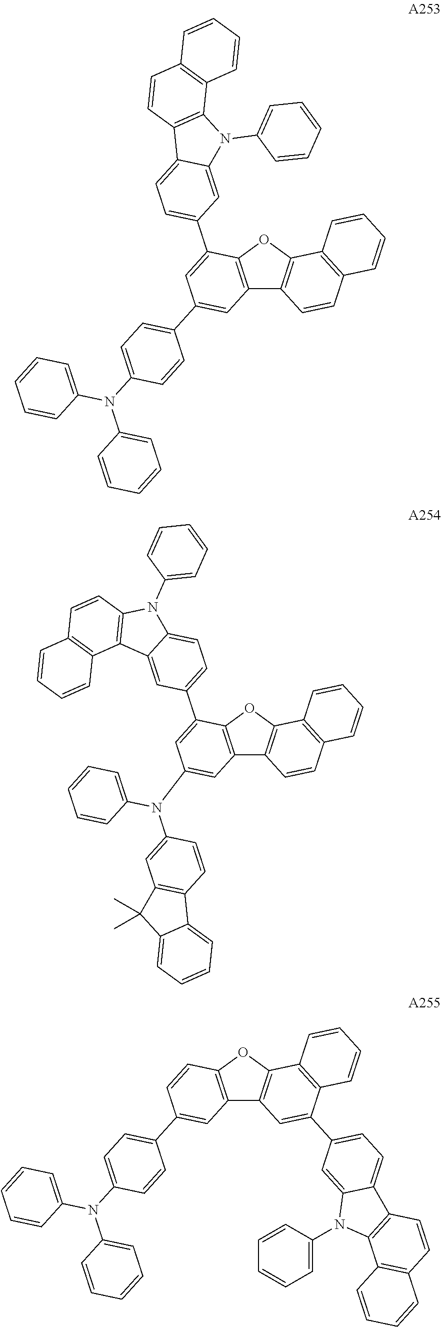

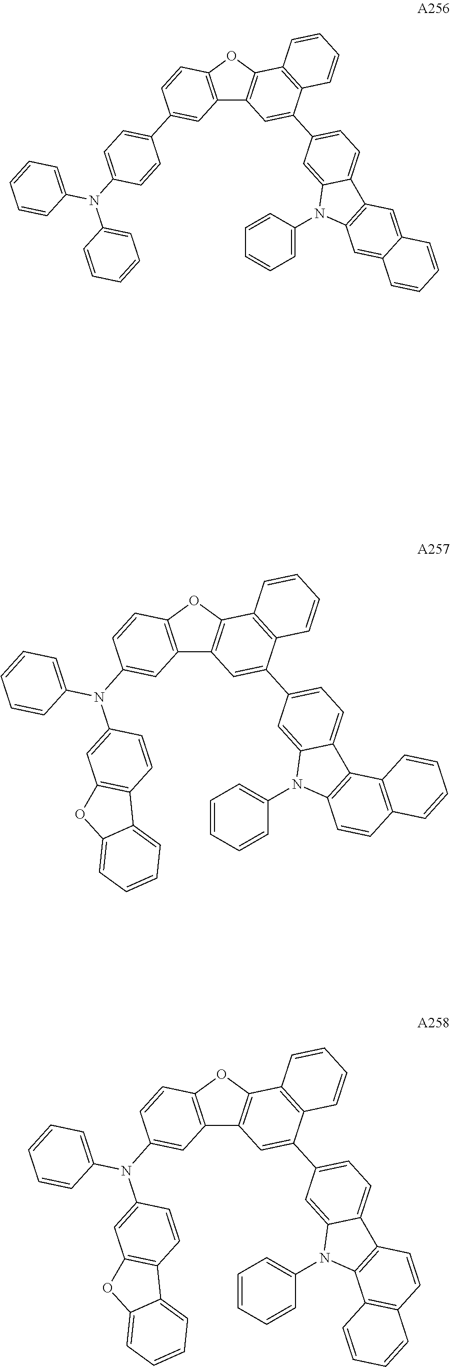 [SMILES: c1ccc(N(c2ccccc2)c2ccc(-c3ccc4oc5c6ccccc6c(-c6ccc7c8cc9ccccc9cc8n(-c8ccccc8)c7c6)cc5c4c3)cc2)cc1 ; c1ccc(N(c2ccc3c(c2)oc2ccccc23)c2ccc3oc4c5ccccc5c(-c5ccc6c7c8ccccc8ccc7n(-c7ccccc7)c6c5)cc4c3c2)cc1 ; c1ccc(N(c2ccc3c(c2)oc2ccccc23)c2ccc3oc4c5ccccc5c(-c5ccc6c7ccc8ccccc8c7n(-c7ccccc7)c6c5)cc4c3c2)cc1]

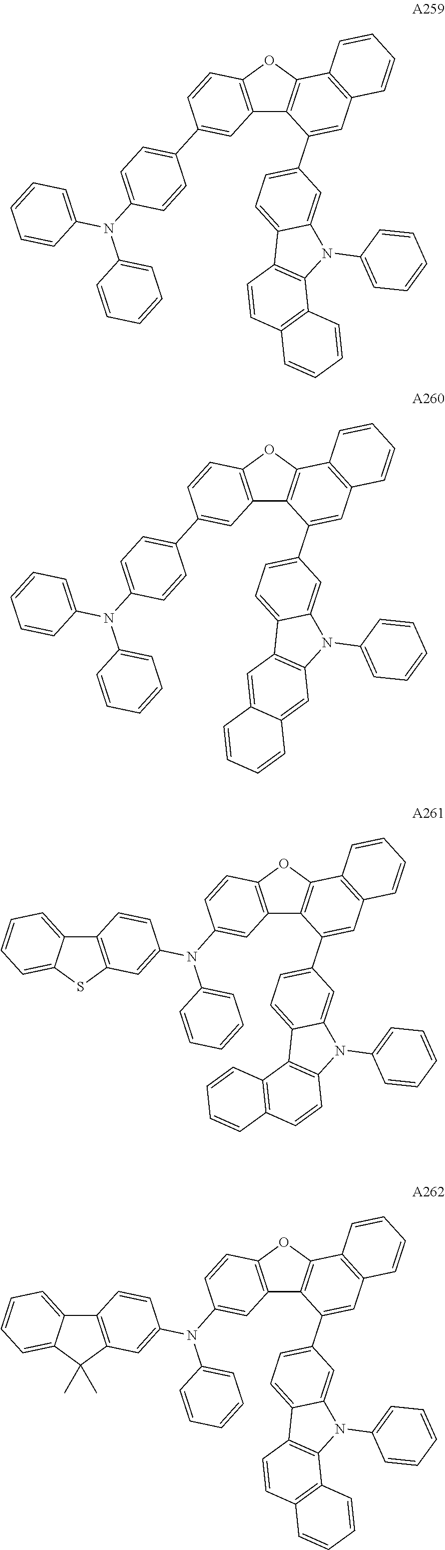 [SMILES: c1ccc(N(c2ccccc2)c2ccc(-c3ccc4oc5c6ccccc6cc(-c6ccc7c8ccc9ccccc9c8n(-c8ccccc8)c7c6)c5c4c3)cc2)cc1 ; c1ccc(N(c2ccccc2)c2ccc(-c3ccc4oc5c6ccccc6cc(-c6ccc7c8cc9ccccc9cc8n(-c8ccccc8)c7c6)c5c4c3)cc2)cc1 ; c1ccc(N(c2ccc3c(c2)sc2ccccc23)c2ccc3oc4c5ccccc5cc(-c5ccc6c7c8ccccc8ccc7n(-c7ccccc7)c6c5)c4c3c2)cc1 ; CC1(C)c2ccccc2-c2ccc(N(c3ccccc3)c3ccc4oc5c6ccccc6cc(-c6ccc7c8ccc9ccccc9c8n(-c8ccccc8)c7c6)c5c4c3)cc21]

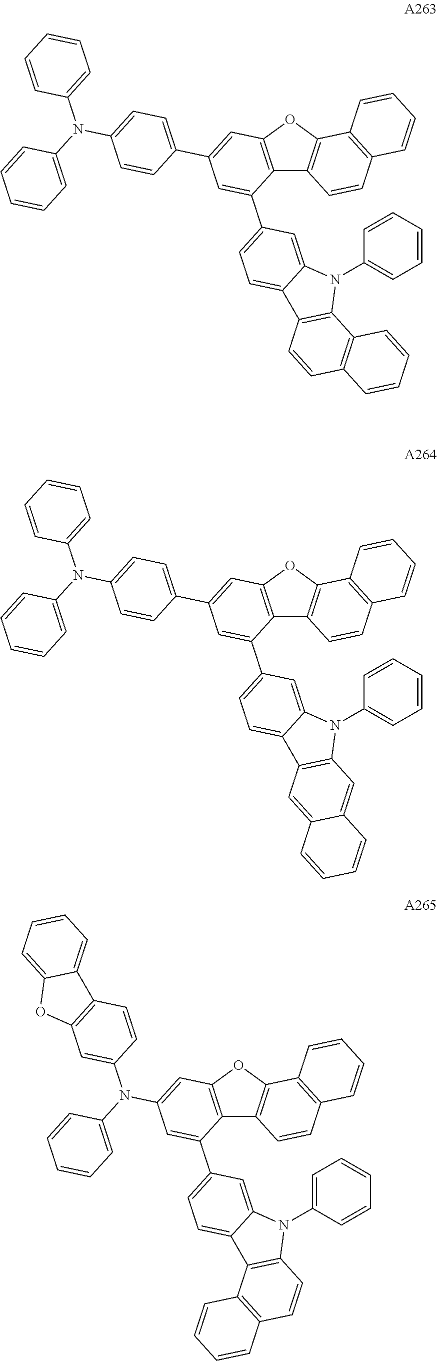 [SMILES: c1ccc(N(c2ccccc2)c2ccc(-c3cc(-c4ccc5c6ccc7ccccc7c6n(-c6ccccc6)c5c4)c4c(c3)oc3c5ccccc5ccc34)cc2)cc1 ; c1ccc(N(c2ccccc2)c2ccc(-c3cc(-c4ccc5c6cc7ccccc7cc6n(-c6ccccc6)c5c4)c4c(c3)oc3c5ccccc5ccc34)cc2)cc1 ; c1ccc(N(c2ccc3c(c2)oc2ccccc23)c2cc(-c3ccc4c5c6ccccc6ccc5n(-c5ccccc5)c4c3)c3c(c2)oc2c4ccccc4ccc23)cc1]

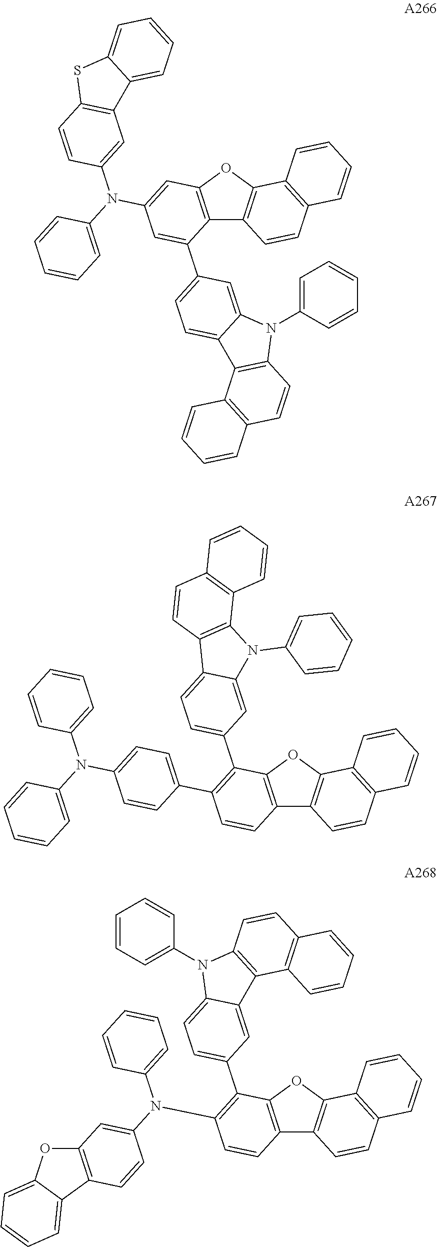 [SMILES: c1ccc(N(c2cc(-c3ccc4c5c6ccccc6ccc5n(-c5ccccc5)c4c3)c3c(c2)oc2c4ccccc4ccc23)c2ccc3sc4ccccc4c3c2)cc1 ; c1ccc(N(c2ccccc2)c2ccc(-c3ccc4c(oc5c6ccccc6ccc45)c3-c3ccc4c5ccc6ccccc6c5n(-c5ccccc5)c4c3)cc2)cc1 ; c1ccc(N(c2ccc3c(c2)oc2ccccc23)c2ccc3c(oc4c5ccccc5ccc34)c2-c2ccc3c(c2)c2c4ccccc4ccc2n3-c2ccccc2)cc1]

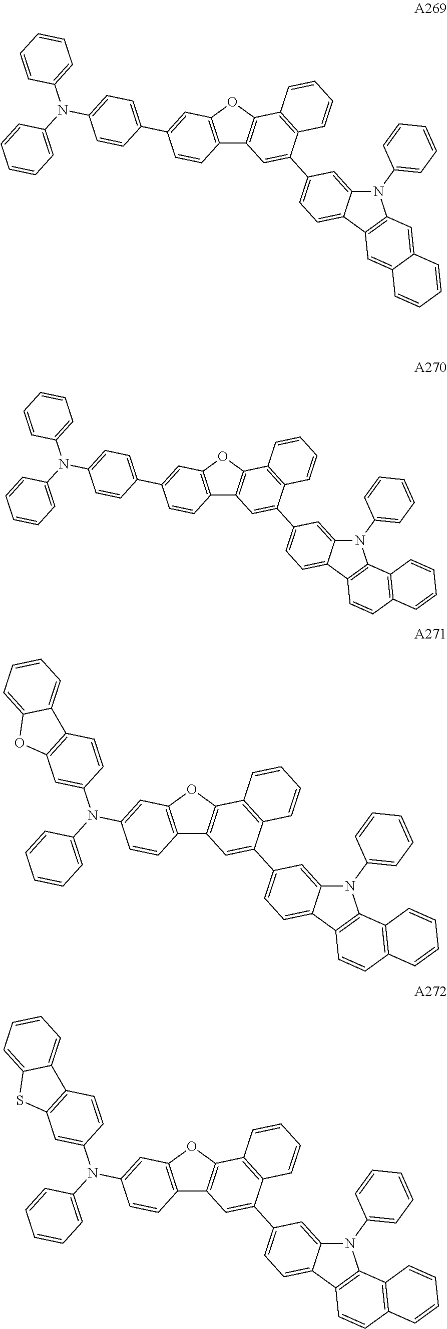 [SMILES: c1ccc(N(c2ccccc2)c2ccc(-c3ccc4c(c3)oc3c5ccccc5c(-c5ccc6c7cc8ccccc8cc7n(-c7ccccc7)c6c5)cc43)cc2)cc1 ; c1ccc(N(c2ccccc2)c2ccc(-c3ccc4c(c3)oc3c5ccccc5c(-c5ccc6c7ccc8ccccc8c7n(-c7ccccc7)c6c5)cc43)cc2)cc1 ; c1ccc(N(c2ccc3c(c2)oc2ccccc23)c2ccc3c(c2)oc2c4ccccc4c(-c4ccc5c6ccc7ccccc7c6n(-c6ccccc6)c5c4)cc32)cc1 ; c1ccc(N(c2ccc3c(c2)oc2c4ccccc4c(-c4ccc5c6ccc7ccccc7c6n(-c6ccccc6)c5c4)cc32)c2ccc3c(c2)sc2ccccc23)cc1]

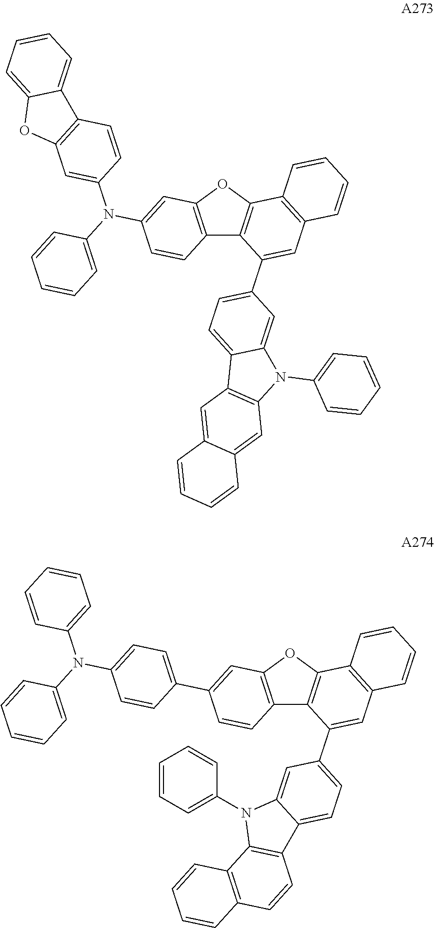 [SMILES: c1ccc(N(c2ccc3c(c2)oc2ccccc23)c2ccc3c(c2)oc2c4ccccc4cc(-c4ccc5c6cc7ccccc7cc6n(-c6ccccc6)c5c4)c32)cc1 ; c1ccc(N(c2ccccc2)c2ccc(-c3ccc4c(c3)oc3c5ccccc5cc(-c5ccc6c7ccc8ccccc8c7n(-c7ccccc7)c6c5)c43)cc2)cc1]

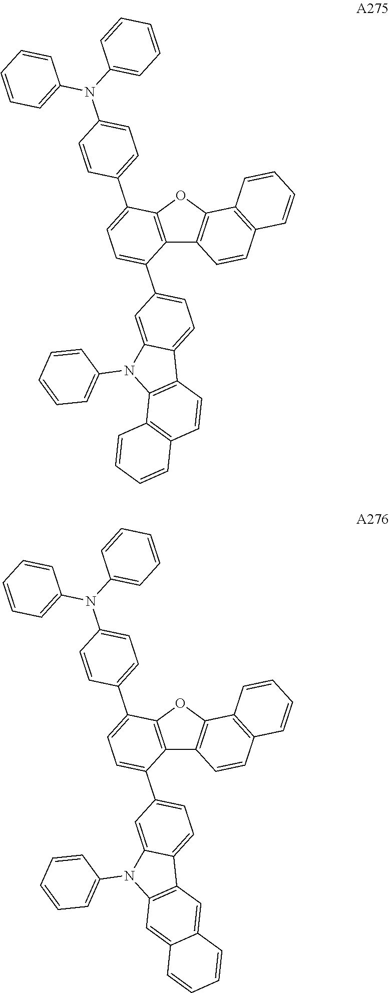 [SMILES: c1ccc(N(c2ccccc2)c2ccc(-c3ccc(-c4ccc5c6ccc7ccccc7c6n(-c6ccccc6)c5c4)c4c3oc3c5ccccc5ccc34)cc2)cc1 ; c1ccc(N(c2ccccc2)c2ccc(-c3ccc(-c4ccc5c6cc7ccccc7cc6n(-c6ccccc6)c5c4)c4c3oc3c5ccccc5ccc34)cc2)cc1]

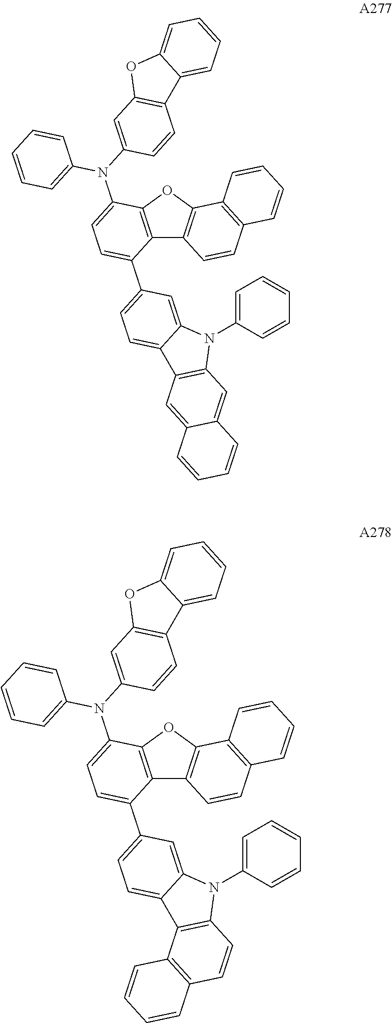 [SMILES: c1ccc(N(c2ccc3c(c2)oc2ccccc23)c2ccc(-c3ccc4c5cc6ccccc6cc5n(-c5ccccc5)c4c3)c3c2oc2c4ccccc4ccc23)cc1 ; c1ccc(N(c2ccc3c(c2)oc2ccccc23)c2ccc(-c3ccc4c5c6ccccc6ccc5n(-c5ccccc5)c4c3)c3c2oc2c4ccccc4ccc23)cc1]

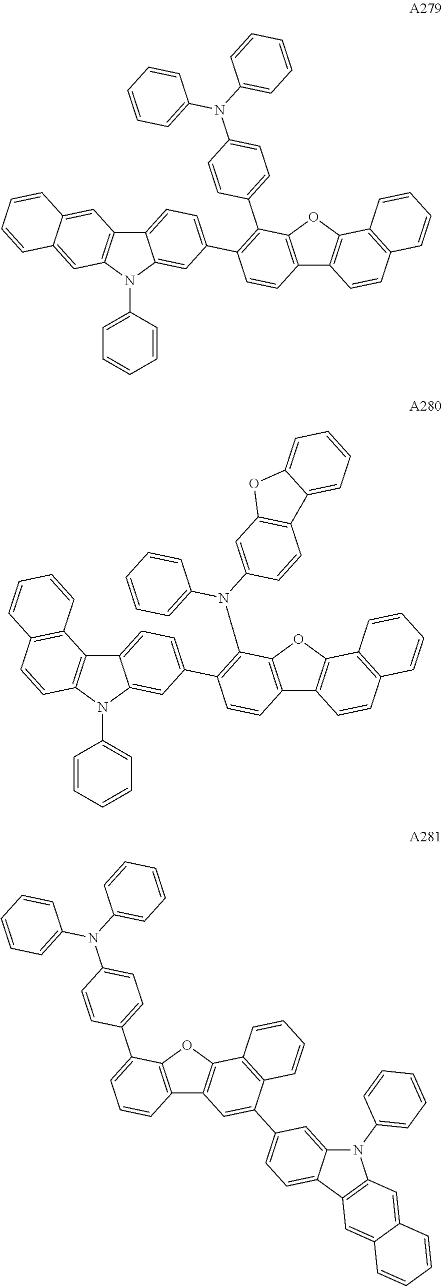 [SMILES: c1ccc(N(c2ccccc2)c2ccc(-c3c(-c4ccc5c6cc7ccccc7cc6n(-c6ccccc6)c5c4)ccc4c3oc3c5ccccc5ccc43)cc2)cc1 ; c1ccc(N(c2ccc3c(c2)oc2ccccc23)c2c(-c3ccc4c5c6ccccc6ccc5n(-c5ccccc5)c4c3)ccc3c2oc2c4ccccc4ccc32)cc1 ; c1ccc(N(c2ccccc2)c2ccc(-c3cccc4c3oc3c5ccccc5c(-c5ccc6c7cc8ccccc8cc7n(-c7ccccc7)c6c5)cc43)cc2)cc1]

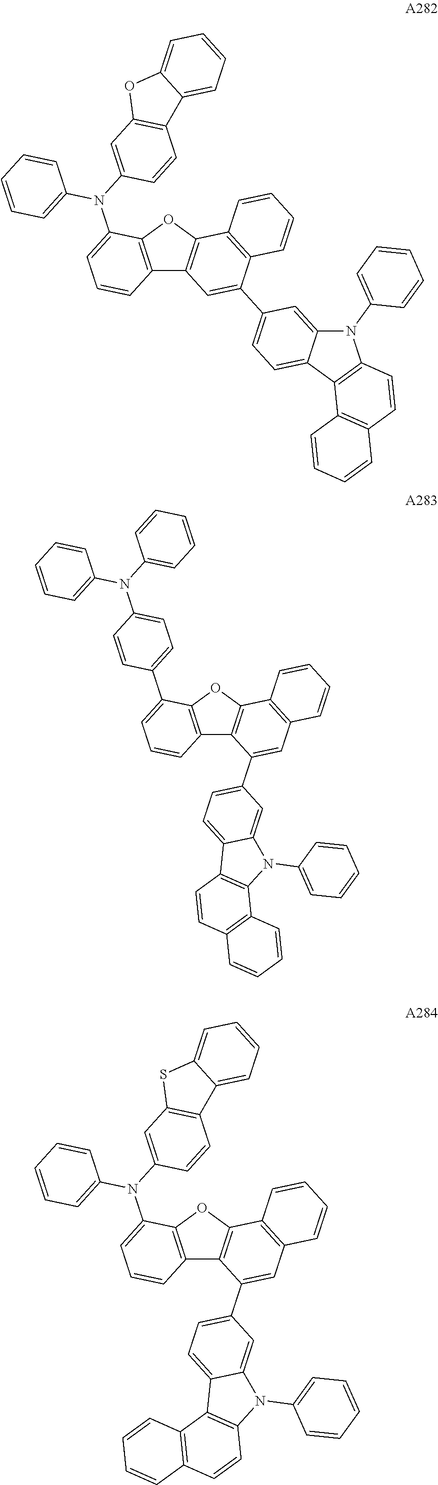 [SMILES: c1ccc(N(c2ccc3c(c2)oc2ccccc23)c2cccc3c2oc2c4ccccc4c(-c4ccc5c6c7ccccc7ccc6n(-c6ccccc6)c5c4)cc32)cc1 ; c1ccc(N(c2ccccc2)c2ccc(-c3cccc4c3oc3c5ccccc5cc(-c5ccc6c7ccc8ccccc8c7n(-c7ccccc7)c6c5)c43)cc2)cc1 ; c1ccc(N(c2ccc3c(c2)sc2ccccc23)c2cccc3c2oc2c4ccccc4cc(-c4ccc5c6c7ccccc7ccc6n(-c6ccccc6)c5c4)c32)cc1]

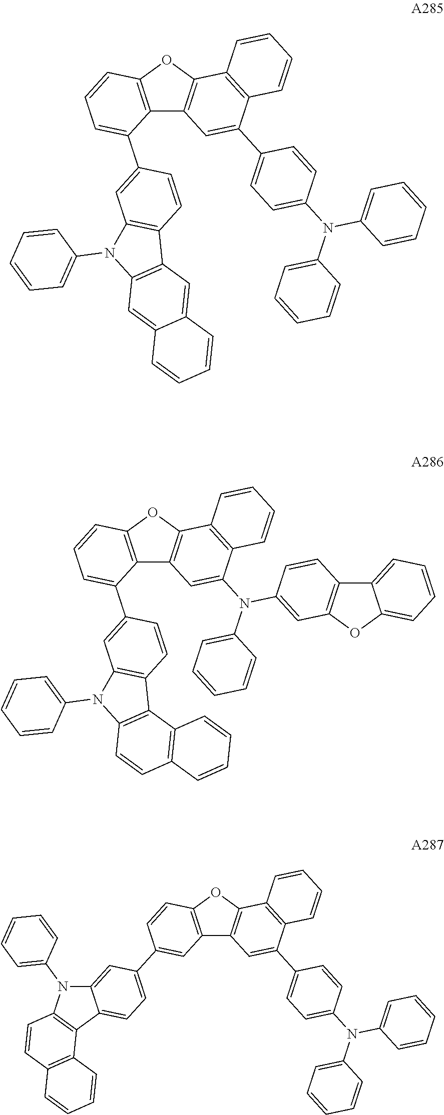 [SMILES: c1ccc(N(c2ccccc2)c2ccc(-c3cc4c(oc5cccc(-c6ccc7c8cc9ccccc9cc8n(-c8ccccc8)c7c6)c54)c4ccccc34)cc2)cc1 ; c1ccc(N(c2ccc3c(c2)oc2ccccc23)c2cc3c(oc4cccc(-c5ccc6c7c8ccccc8ccc7n(-c7ccccc7)c6c5)c43)c3ccccc23)cc1 ; c1ccc(N(c2ccccc2)c2ccc(-c3cc4c5cc(-c6ccc7c8c9ccccc9ccc8n(-c8ccccc8)c7c6)ccc5oc4c4ccccc34)cc2)cc1]

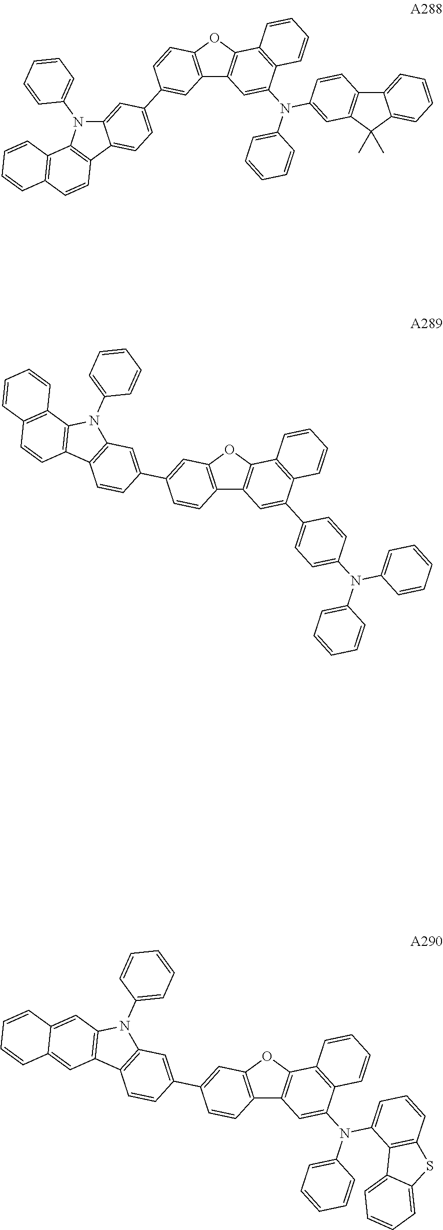 [SMILES: CC1(C)c2ccccc2-c2ccc(N(c3ccccc3)c3cc4c5cc(-c6ccc7c8ccc9ccccc9c8n(-c8ccccc8)c7c6)ccc5oc4c4ccccc34)cc21 ; c1ccc(N(c2ccccc2)c2ccc(-c3cc4c5ccc(-c6ccc7c8ccc9ccccc9c8n(-c8ccccc8)c7c6)cc5oc4c4ccccc34)cc2)cc1 ; c1ccc(N(c2cc3c4ccc(-c5ccc6c7cc8ccccc8cc7n(-c7ccccc7)c6c5)cc4oc3c3ccccc23)c2cccc3sc4ccccc4c23)cc1]

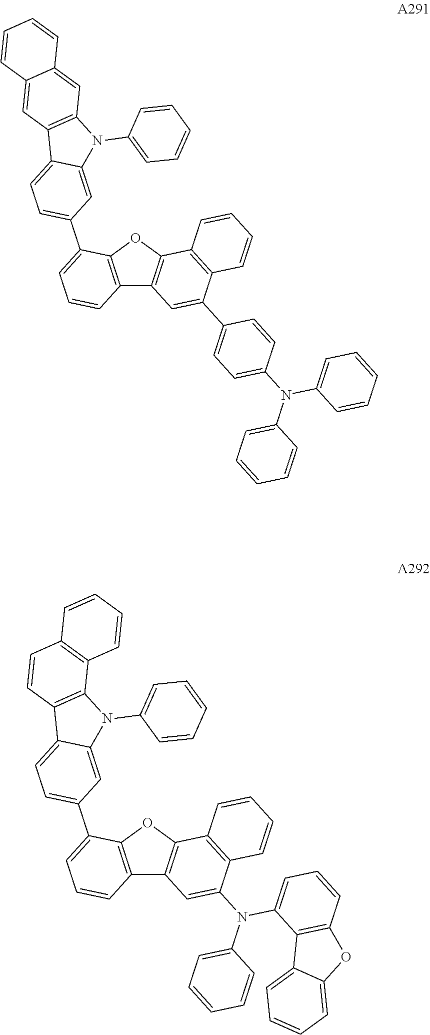 [SMILES: c1ccc(N(c2ccccc2)c2ccc(-c3cc4c5cccc(-c6ccc7c8cc9ccccc9cc8n(-c8ccccc8)c7c6)c5oc4c4ccccc34)cc2)cc1 ; c1ccc(N(c2cc3c4cccc(-c5ccc6c7ccc8ccccc8c7n(-c7ccccc7)c6c5)c4oc3c3ccccc23)c2cccc3oc4ccccc4c23)cc1]

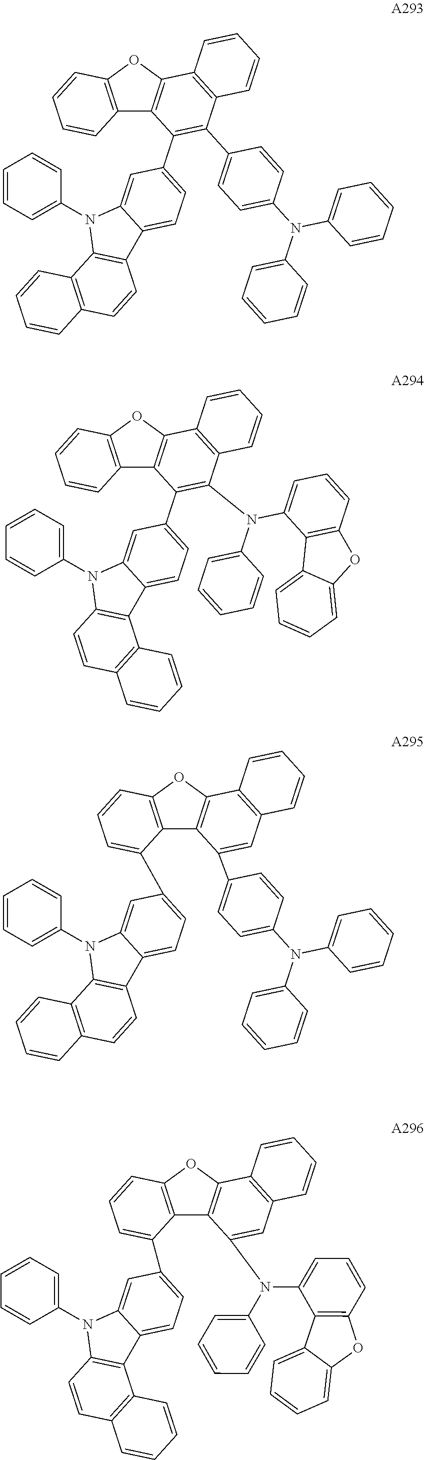 [SMILES: c1ccc(N(c2ccccc2)c2ccc(-c3c(-c4ccc5c6ccc7ccccc7c6n(-c6ccccc6)c5c4)c4c5ccccc5oc4c4ccccc34)cc2)cc1 ; c1ccc(N(c2c(-c3ccc4c5c6ccccc6ccc5n(-c5ccccc5)c4c3)c3c4ccccc4oc3c3ccccc23)c2cccc3oc4ccccc4c23)cc1 ; c1ccc(N(c2ccccc2)c2ccc(-c3cc4ccccc4c4oc5cccc(-c6ccc7c8ccc9ccccc9c8n(-c8ccccc8)c7c6)c5c34)cc2)cc1 ; c1ccc(N(c2cccc3oc4ccccc4c23)c2cc3ccccc3c3oc4cccc(-c5ccc6c7c8ccccc8ccc7n(-c7ccccc7)c6c5)c4c23)cc1]

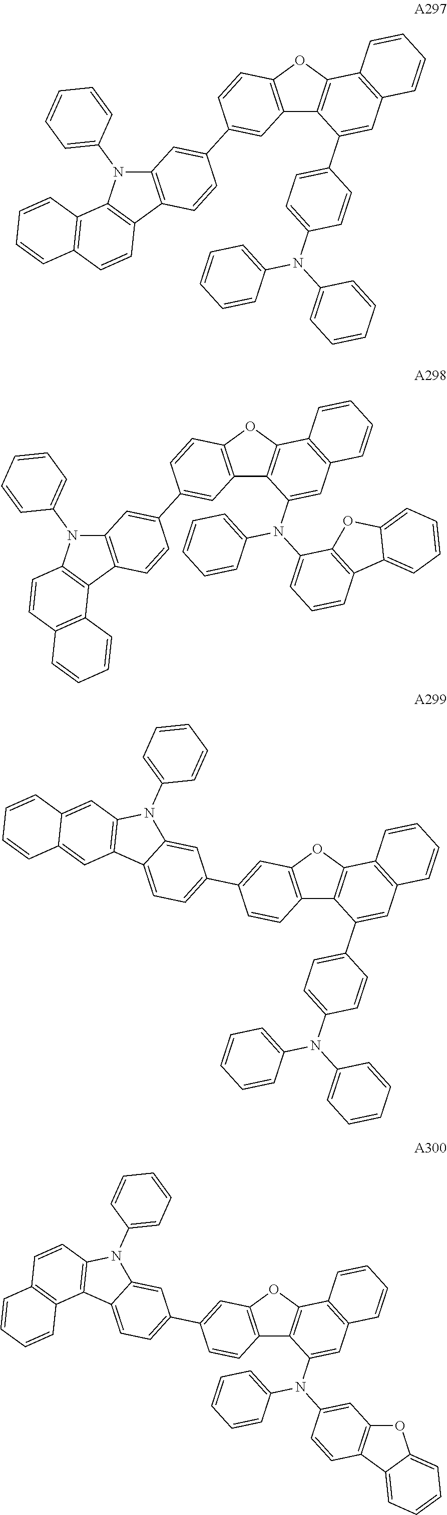 [SMILES: c1ccc(N(c2ccccc2)c2ccc(-c3cc4ccccc4c4oc5ccc(-c6ccc7c8ccc9ccccc9c8n(-c8ccccc8)c7c6)cc5c34)cc2)cc1 ; c1ccc(N(c2cccc3c2oc2ccccc23)c2cc3ccccc3c3oc4ccc(-c5ccc6c7c8ccccc8ccc7n(-c7ccccc7)c6c5)cc4c23)cc1 ; c1ccc(N(c2ccccc2)c2ccc(-c3cc4ccccc4c4oc5cc(-c6ccc7c8cc9ccccc9cc8n(-c8ccccc8)c7c6)ccc5c34)cc2)cc1 ; c1ccc(N(c2ccc3c(c2)oc2ccccc23)c2cc3ccccc3c3oc4cc(-c5ccc6c7c8ccccc8ccc7n(-c7ccccc7)c6c5)ccc4c23)cc1]

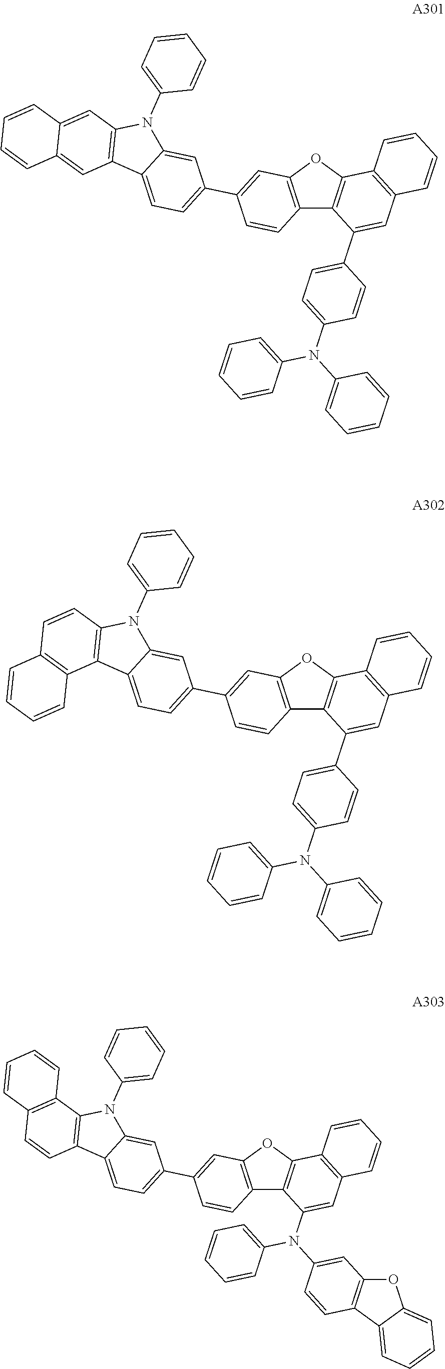 [SMILES: c1ccc(N(c2ccccc2)c2ccc(-c3cc4ccccc4c4oc5cc(-c6ccc7c8cc9ccccc9cc8n(-c8ccccc8)c7c6)ccc5c34)cc2)cc1 ; c1ccc(N(c2ccccc2)c2ccc(-c3cc4ccccc4c4oc5cc(-c6ccc7c8c9ccccc9ccc8n(-c8ccccc8)c7c6)ccc5c34)cc2)cc1 ; c1ccc(N(c2ccc3c(c2)oc2ccccc23)c2cc3ccccc3c3oc4cc(-c5ccc6c7ccc8ccccc8c7n(-c7ccccc7)c6c5)ccc4c23)cc1]

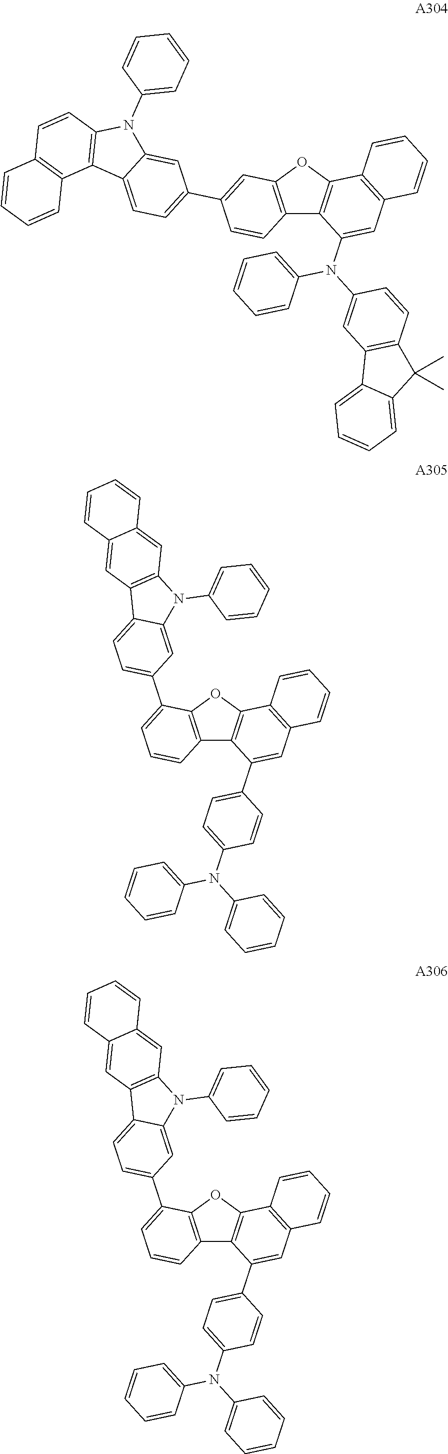 [SMILES: CC1(C)c2ccccc2-c2cc(N(c3ccccc3)c3cc4ccccc4c4oc5cc(-c6ccc7c8c9ccccc9ccc8n(-c8ccccc8)c7c6)ccc5c34)ccc21 ; c1ccc(N(c2ccccc2)c2ccc(-c3cc4ccccc4c4oc5c(-c6ccc7c8cc9ccccc9cc8n(-c8ccccc8)c7c6)cccc5c34)cc2)cc1 ; c1ccc(N(c2ccccc2)c2ccc(-c3cc4ccccc4c4oc5c(-c6ccc7c8cc9ccccc9cc8n(-c8ccccc8)c7c6)cccc5c34)cc2)cc1]

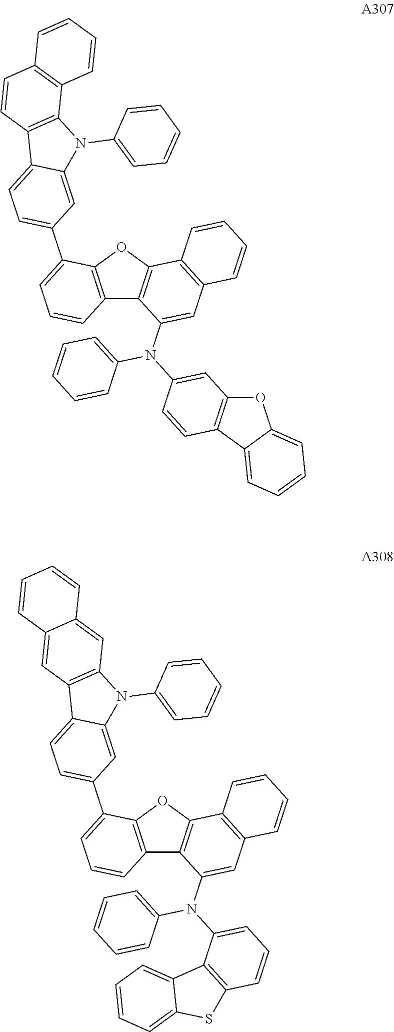 [SMILES: c1ccc(N(c2ccc3c(c2)oc2ccccc23)c2cc3ccccc3c3oc4c(-c5ccc6c7ccc8ccccc8c7n(-c7ccccc7)c6c5)cccc4c23)cc1 ; c1ccc(N(c2cccc3sc4ccccc4c23)c2cc3ccccc3c3oc4c(-c5ccc6c7cc8ccccc8cc7n(-c7ccccc7)c6c5)cccc4c23)cc1]

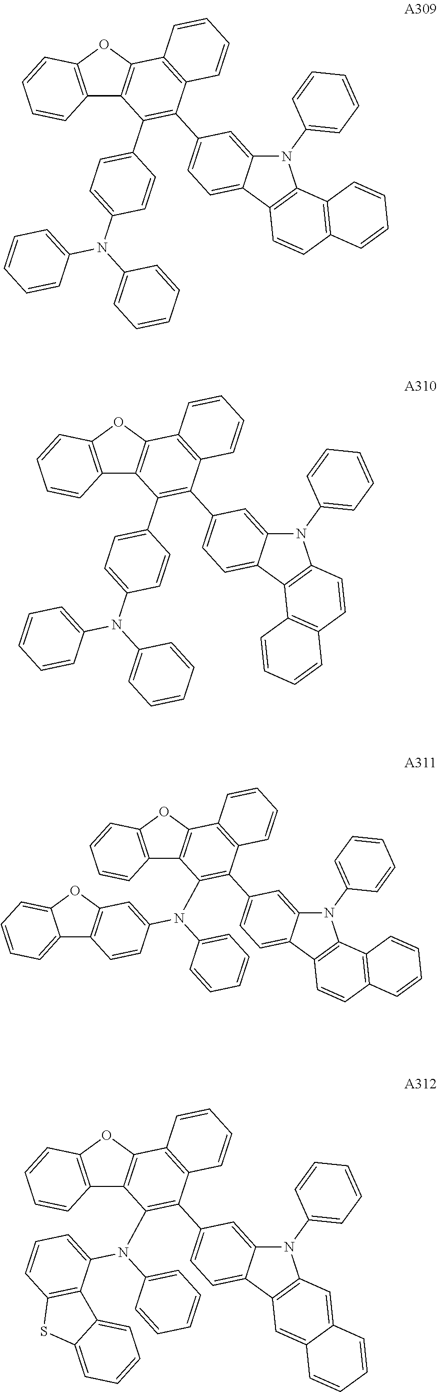 [SMILES: c1ccc(N(c2ccccc2)c2ccc(-c3c(-c4ccc5c6ccc7ccccc7c6n(-c6ccccc6)c5c4)c4ccccc4c4oc5ccccc5c34)cc2)cc1 ; c1ccc(N(c2ccccc2)c2ccc(-c3c(-c4ccc5c6c7ccccc7ccc6n(-c6ccccc6)c5c4)c4ccccc4c4oc5ccccc5c34)cc2)cc1 ; c1ccc(N(c2ccc3c(c2)oc2ccccc23)c2c(-c3ccc4c5ccc6ccccc6c5n(-c5ccccc5)c4c3)c3ccccc3c3oc4ccccc4c23)cc1 ; c1ccc(N(c2cccc3sc4ccccc4c23)c2c(-c3ccc4c5cc6ccccc6cc5n(-c5ccccc5)c4c3)c3ccccc3c3oc4ccccc4c23)cc1]

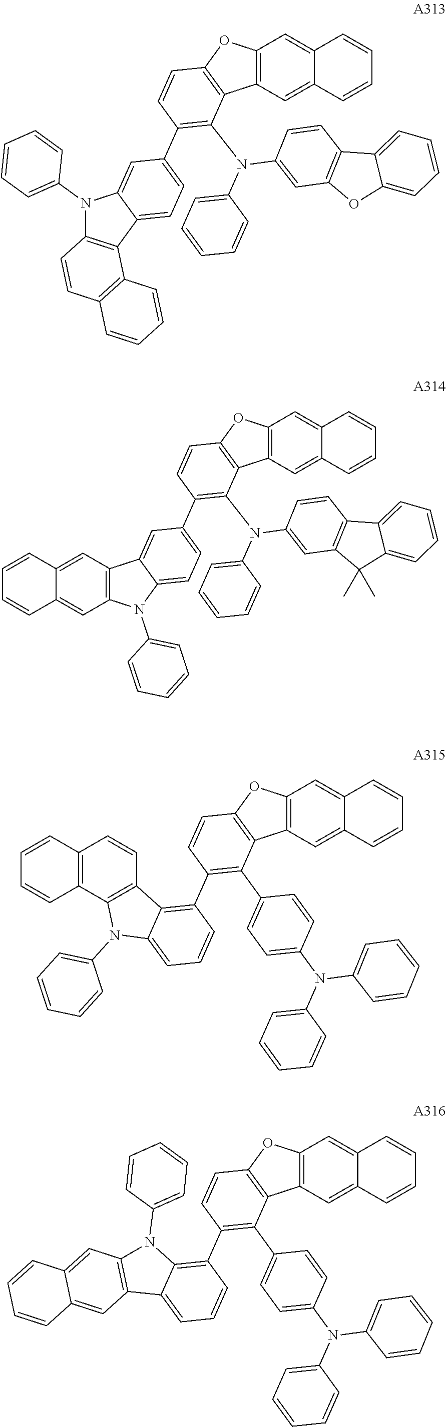 [SMILES: c1ccc(N(c2ccc3c(c2)oc2ccccc23)c2c(-c3ccc4c5c6ccccc6ccc5n(-c5ccccc5)c4c3)ccc3oc4cc5ccccc5cc4c23)cc1 ; CC1(C)c2ccccc2-c2ccc(N(c3ccccc3)c3c(-c4ccc5c(c4)c4cc6ccccc6cc4n5-c4ccccc4)ccc4oc5cc6ccccc6cc5c34)cc21 ; c1ccc(N(c2ccccc2)c2ccc(-c3c(-c4cccc5c4c4ccc6ccccc6c4n5-c4ccccc4)ccc4oc5cc6ccccc6cc5c34)cc2)cc1 ; c1ccc(N(c2ccccc2)c2ccc(-c3c(-c4cccc5c6cc7ccccc7cc6n(-c6ccccc6)c45)ccc4oc5cc6ccccc6cc5c34)cc2)cc1]

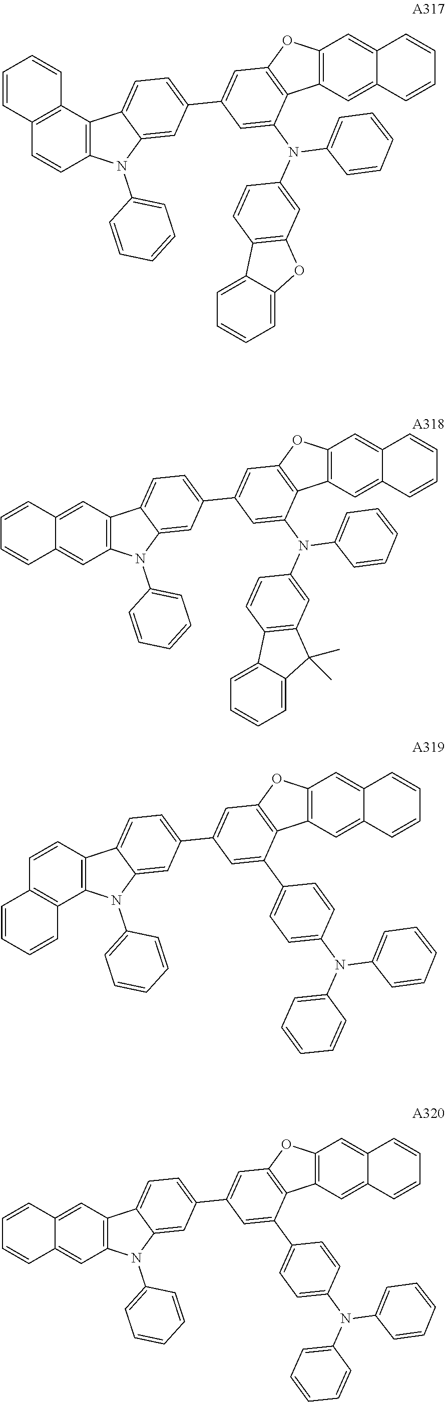 [SMILES: c1ccc(N(c2ccc3c(c2)oc2ccccc23)c2cc(-c3ccc4c5c6ccccc6ccc5n(-c5ccccc5)c4c3)cc3oc4cc5ccccc5cc4c23)cc1 ; CC1(C)c2ccccc2-c2ccc(N(c3ccccc3)c3cc(-c4ccc5c6cc7ccccc7cc6n(-c6ccccc6)c5c4)cc4oc5cc6ccccc6cc5c34)cc21 ; c1ccc(N(c2ccccc2)c2ccc(-c3cc(-c4ccc5c6ccc7ccccc7c6n(-c6ccccc6)c5c4)cc4oc5cc6ccccc6cc5c34)cc2)cc1 ; c1ccc(N(c2ccccc2)c2ccc(-c3cc(-c4ccc5c6cc7ccccc7cc6n(-c6ccccc6)c5c4)cc4oc5cc6ccccc6cc5c34)cc2)cc1]

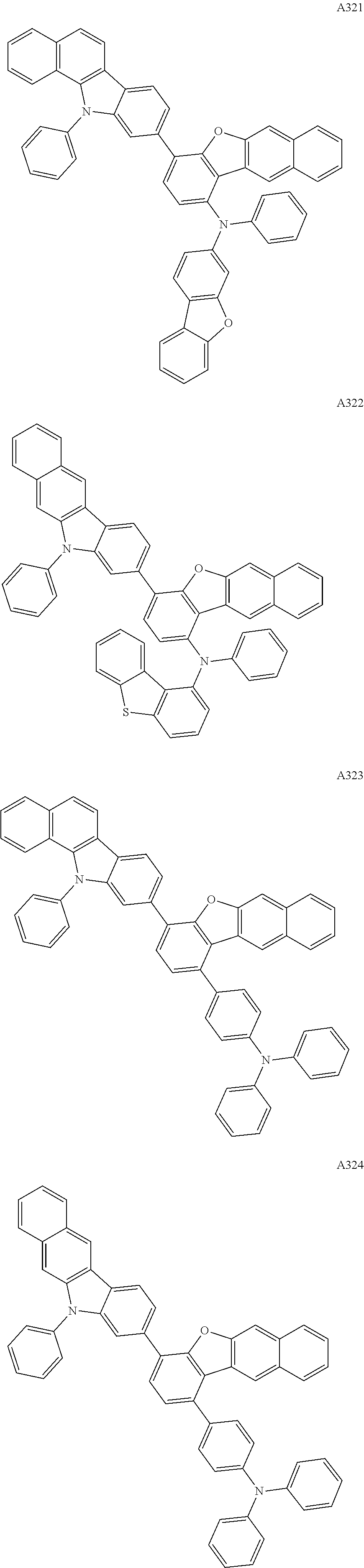 [SMILES: c1ccc(N(c2ccc3c(c2)oc2ccccc23)c2ccc(-c3ccc4c5ccc6ccccc6c5n(-c5ccccc5)c4c3)c3oc4cc5ccccc5cc4c23)cc1 ; c1ccc(N(c2cccc3sc4ccccc4c23)c2ccc(-c3ccc4c5cc6ccccc6cc5n(-c5ccccc5)c4c3)c3oc4cc5ccccc5cc4c23)cc1 ; c1ccc(N(c2ccccc2)c2ccc(-c3ccc(-c4ccc5c6ccc7ccccc7c6n(-c6ccccc6)c5c4)c4oc5cc6ccccc6cc5c34)cc2)cc1 ; c1ccc(N(c2ccccc2)c2ccc(-c3ccc(-c4ccc5c6cc7ccccc7cc6n(-c6ccccc6)c5c4)c4oc5cc6ccccc6cc5c34)cc2)cc1]

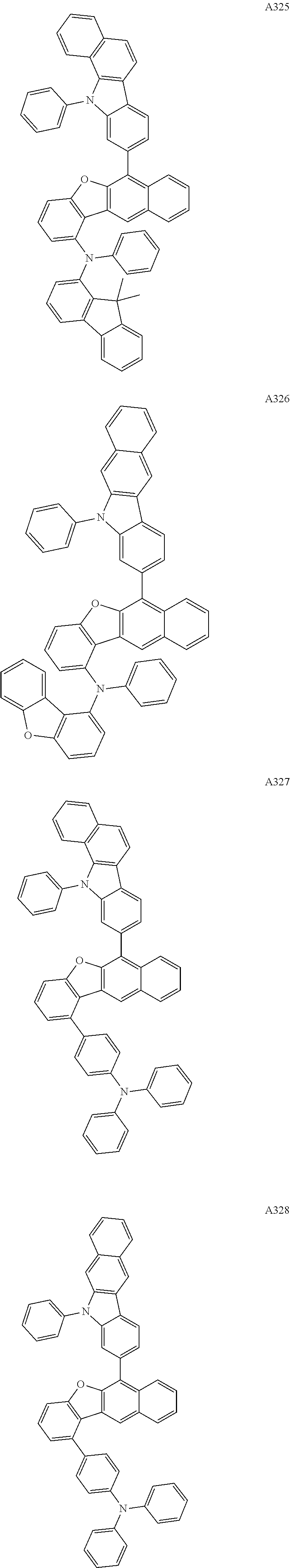 [SMILES: CC1(C)c2ccccc2-c2cccc(N(c3ccccc3)c3cccc4oc5c(-c6ccc7c8ccc9ccccc9c8n(-c8ccccc8)c7c6)c6ccccc6cc5c34)c21 ; c1ccc(N(c2cccc3oc4ccccc4c23)c2cccc3oc4c(-c5ccc6c7cc8ccccc8cc7n(-c7ccccc7)c6c5)c5ccccc5cc4c23)cc1 ; c1ccc(N(c2ccccc2)c2ccc(-c3cccc4oc5c(-c6ccc7c8ccc9ccccc9c8n(-c8ccccc8)c7c6)c6ccccc6cc5c34)cc2)cc1 ; c1ccc(N(c2ccccc2)c2ccc(-c3cccc4oc5c(-c6ccc7c8cc9ccccc9cc8n(-c8ccccc8)c7c6)c6ccccc6cc5c34)cc2)cc1]

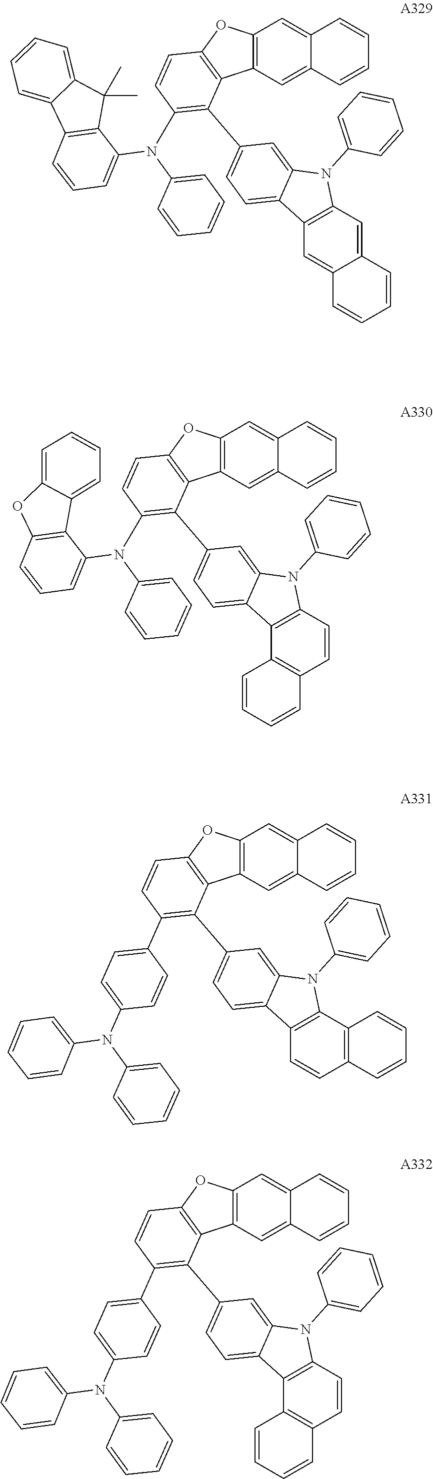 [SMILES: CC1(C)c2ccccc2-c2cccc(N(c3ccccc3)c3ccc4oc5cc6ccccc6cc5c4c3-c3ccc4c5cc6ccccc6cc5n(-c5ccccc5)c4c3)c21 ; c1ccc(N(c2ccc3oc4cc5ccccc5cc4c3c2-c2ccc3c4c5ccccc5ccc4n(-c4ccccc4)c3c2)c2cccc3oc4ccccc4c23)cc1 ; c1ccc(N(c2ccccc2)c2ccc(-c3ccc4oc5cc6ccccc6cc5c4c3-c3ccc4c5ccc6ccccc6c5n(-c5ccccc5)c4c3)cc2)cc1 ; c1ccc(N(c2ccccc2)c2ccc(-c3ccc4oc5cc6ccccc6cc5c4c3-c3ccc4c5c6ccccc6ccc5n(-c5ccccc5)c4c3)cc2)cc1]

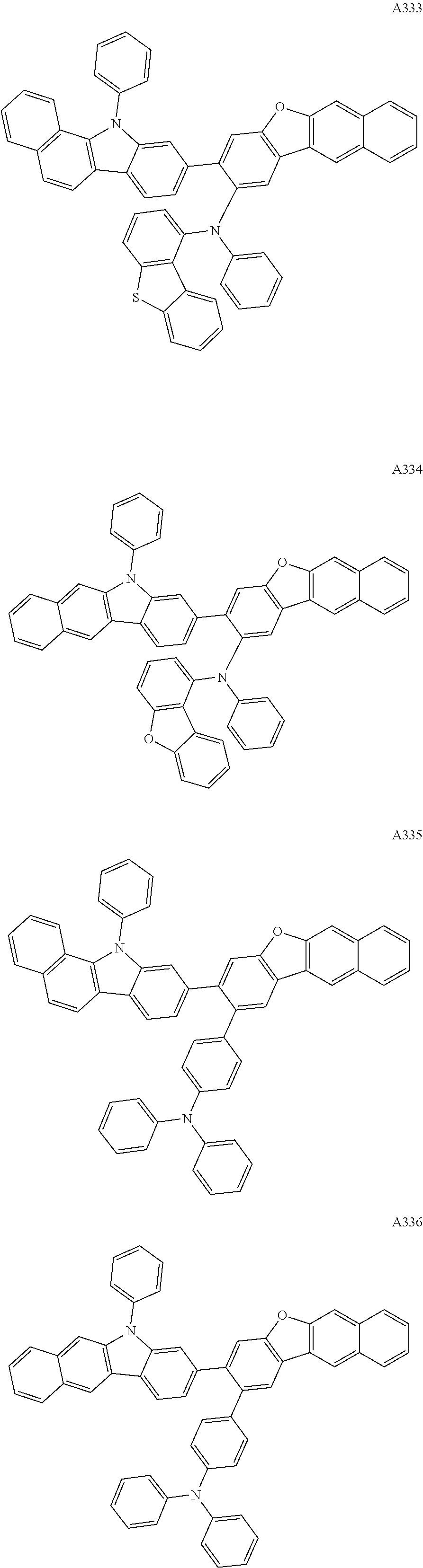 [SMILES: c1ccc(N(c2cc3c(cc2-c2ccc4c5ccc6ccccc6c5n(-c5ccccc5)c4c2)oc2cc4ccccc4cc23)c2cccc3sc4ccccc4c23)cc1 ; c1ccc(N(c2cc3c(cc2-c2ccc4c5cc6ccccc6cc5n(-c5ccccc5)c4c2)oc2cc4ccccc4cc23)c2cccc3oc4ccccc4c23)cc1 ; c1ccc(N(c2ccccc2)c2ccc(-c3cc4c(cc3-c3ccc5c6ccc7ccccc7c6n(-c6ccccc6)c5c3)oc3cc5ccccc5cc34)cc2)cc1 ; c1ccc(N(c2ccccc2)c2ccc(-c3cc4c(cc3-c3ccc5c6cc7ccccc7cc6n(-c6ccccc6)c5c3)oc3cc5ccccc5cc34)cc2)cc1]

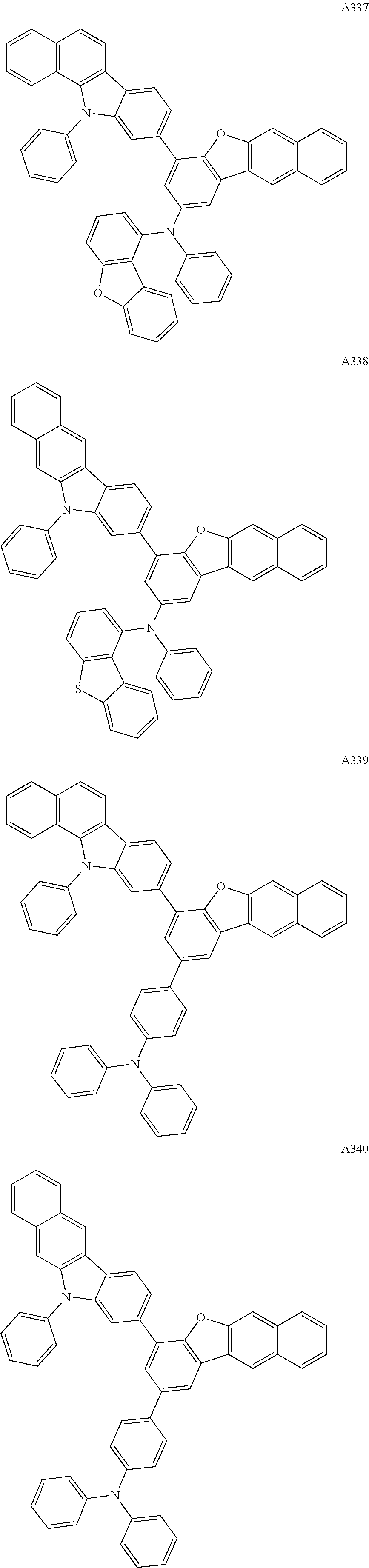 [SMILES: c1ccc(N(c2cc(-c3ccc4c5ccc6ccccc6c5n(-c5ccccc5)c4c3)c3oc4cc5ccccc5cc4c3c2)c2cccc3oc4ccccc4c23)cc1 ; c1ccc(N(c2cc(-c3ccc4c5cc6ccccc6cc5n(-c5ccccc5)c4c3)c3oc4cc5ccccc5cc4c3c2)c2cccc3sc4ccccc4c23)cc1 ; c1ccc(N(c2ccccc2)c2ccc(-c3cc(-c4ccc5c6ccc7ccccc7c6n(-c6ccccc6)c5c4)c4oc5cc6ccccc6cc5c4c3)cc2)cc1 ; c1ccc(N(c2ccccc2)c2ccc(-c3cc(-c4ccc5c6cc7ccccc7cc6n(-c6ccccc6)c5c4)c4oc5cc6ccccc6cc5c4c3)cc2)cc1]

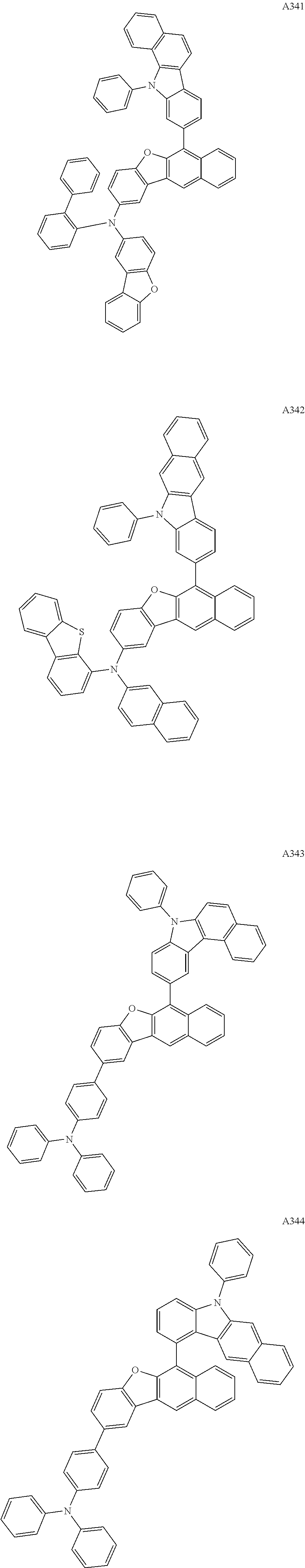 [SMILES: c1ccc(-c2ccccc2N(c2ccc3oc4ccccc4c3c2)c2ccc3oc4c(-c5ccc6c7ccc8ccccc8c7n(-c7ccccc7)c6c5)c5ccccc5cc4c3c2)cc1 ; c1ccc(-n2c3cc(-c4c5ccccc5cc5c4oc4ccc(N(c6ccc7ccccc7c6)c6cccc7c6sc6ccccc67)cc45)ccc3c3cc4ccccc4cc32)cc1 ; c1ccc(N(c2ccccc2)c2ccc(-c3ccc4oc5c(-c6ccc7c(c6)c6c8ccccc8ccc6n7-c6ccccc6)c6ccccc6cc5c4c3)cc2)cc1 ; c1ccc(N(c2ccccc2)c2ccc(-c3ccc4oc5c(-c6cccc7c6c6cc8ccccc8cc6n7-c6ccccc6)c6ccccc6cc5c4c3)cc2)cc1]

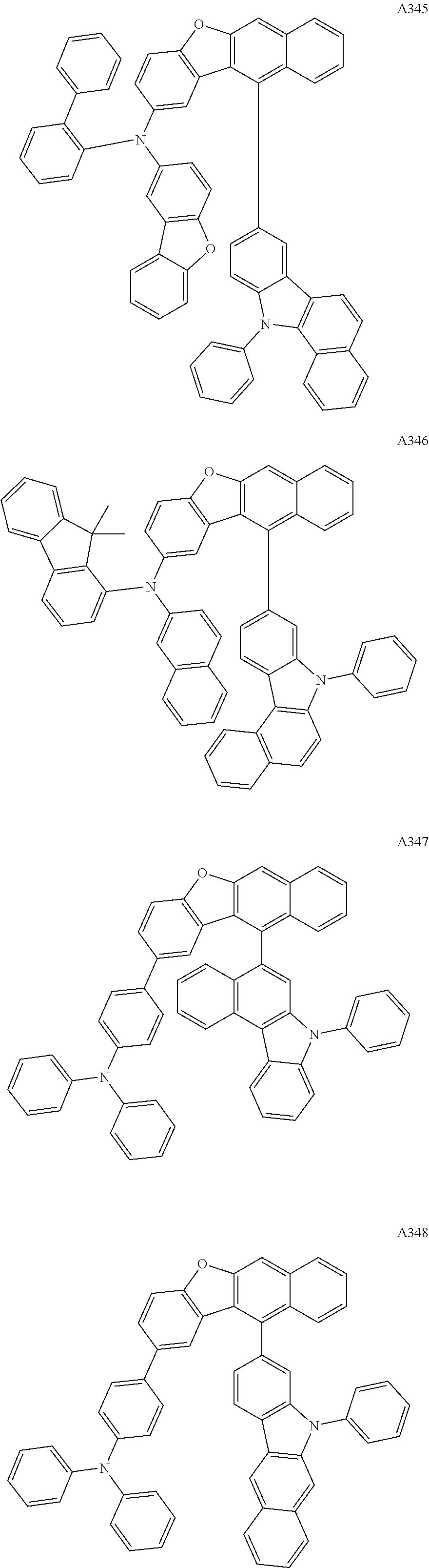 [SMILES: c1ccc(-c2ccccc2N(c2ccc3oc4ccccc4c3c2)c2ccc3oc4cc5ccccc5c(-c5ccc6c(c5)c5ccc7ccccc7c5n6-c5ccccc5)c4c3c2)cc1 ; CC1(C)c2ccccc2-c2cccc(N(c3ccc4ccccc4c3)c3ccc4oc5cc6ccccc6c(-c6ccc7c8c9ccccc9ccc8n(-c8ccccc8)c7c6)c5c4c3)c21 ; c1ccc(N(c2ccccc2)c2ccc(-c3ccc4oc5cc6ccccc6c(-c6cc7c(c8ccccc68)c6ccccc6n7-c6ccccc6)c5c4c3)cc2)cc1 ; c1ccc(N(c2ccccc2)c2ccc(-c3ccc4oc5cc6ccccc6c(-c6ccc7c8cc9ccccc9cc8n(-c8ccccc8)c7c6)c5c4c3)cc2)cc1]

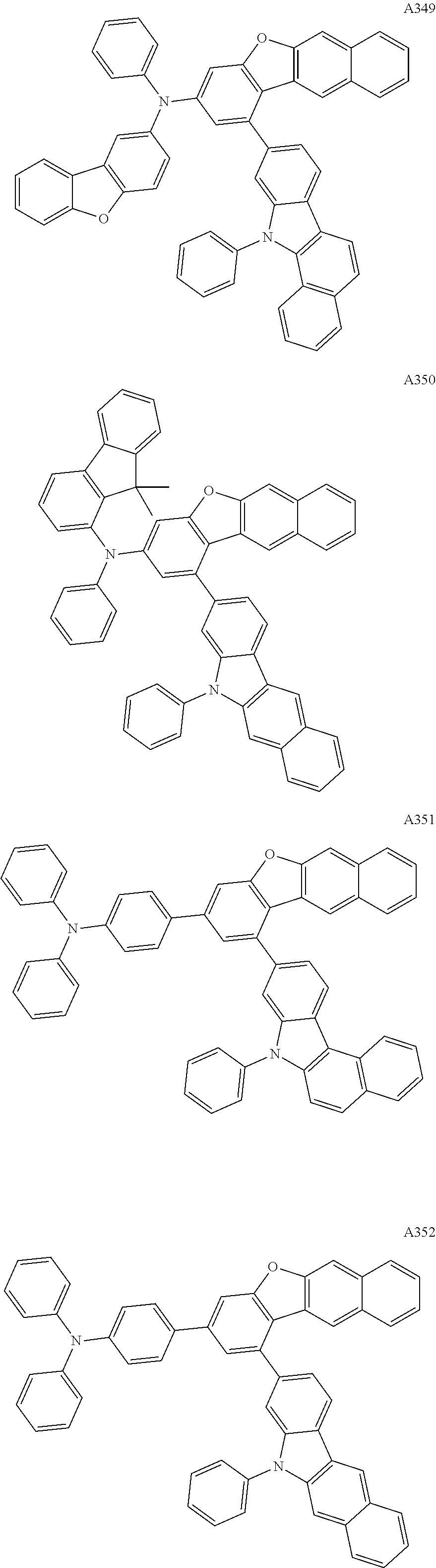 [SMILES: c1ccc(N(c2cc(-c3ccc4c5ccc6ccccc6c5n(-c5ccccc5)c4c3)c3c(c2)oc2cc4ccccc4cc23)c2ccc3oc4ccccc4c3c2)cc1 ; CC1(C)c2ccccc2-c2cccc(N(c3ccccc3)c3cc(-c4ccc5c6cc7ccccc7cc6n(-c6ccccc6)c5c4)c4c(c3)oc3cc5ccccc5cc34)c21 ; c1ccc(N(c2ccccc2)c2ccc(-c3cc(-c4ccc5c6c7ccccc7ccc6n(-c6ccccc6)c5c4)c4c(c3)oc3cc5ccccc5cc34)cc2)cc1 ; c1ccc(N(c2ccccc2)c2ccc(-c3cc(-c4ccc5c6cc7ccccc7cc6n(-c6ccccc6)c5c4)c4c(c3)oc3cc5ccccc5cc34)cc2)cc1]

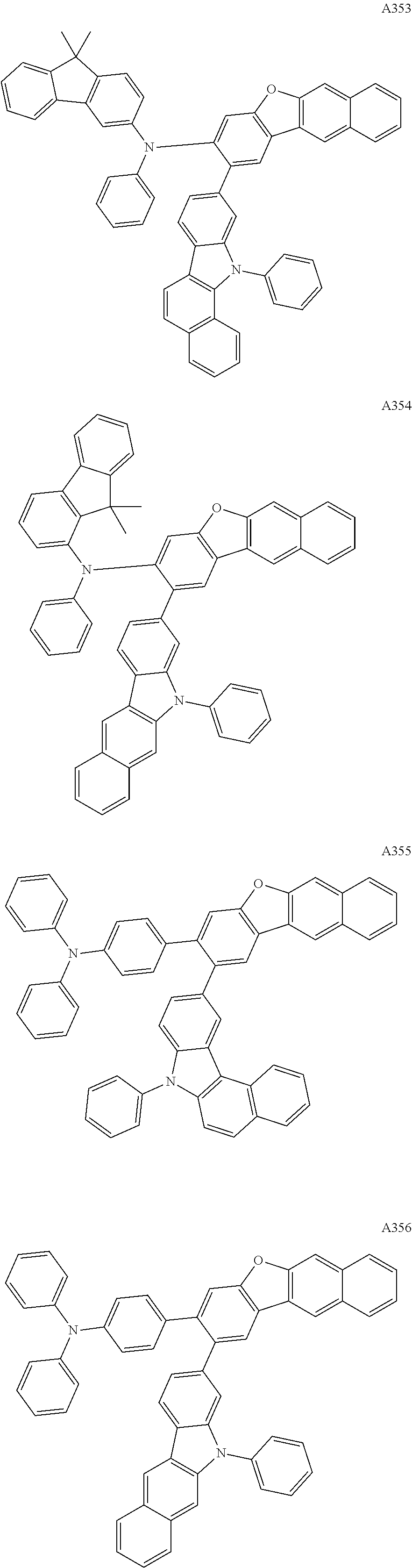 [SMILES: CC1(C)c2ccccc2-c2cc(N(c3ccccc3)c3cc4oc5cc6ccccc6cc5c4cc3-c3ccc4c5ccc6ccccc6c5n(-c5ccccc5)c4c3)ccc21 ; CC1(C)c2ccccc2-c2cccc(N(c3ccccc3)c3cc4oc5cc6ccccc6cc5c4cc3-c3ccc4c5cc6ccccc6cc5n(-c5ccccc5)c4c3)c21 ; c1ccc(N(c2ccccc2)c2ccc(-c3cc4oc5cc6ccccc6cc5c4cc3-c3ccc4c(c3)c3c5ccccc5ccc3n4-c3ccccc3)cc2)cc1 ; c1ccc(N(c2ccccc2)c2ccc(-c3cc4oc5cc6ccccc6cc5c4cc3-c3ccc4c5cc6ccccc6cc5n(-c5ccccc5)c4c3)cc2)cc1]

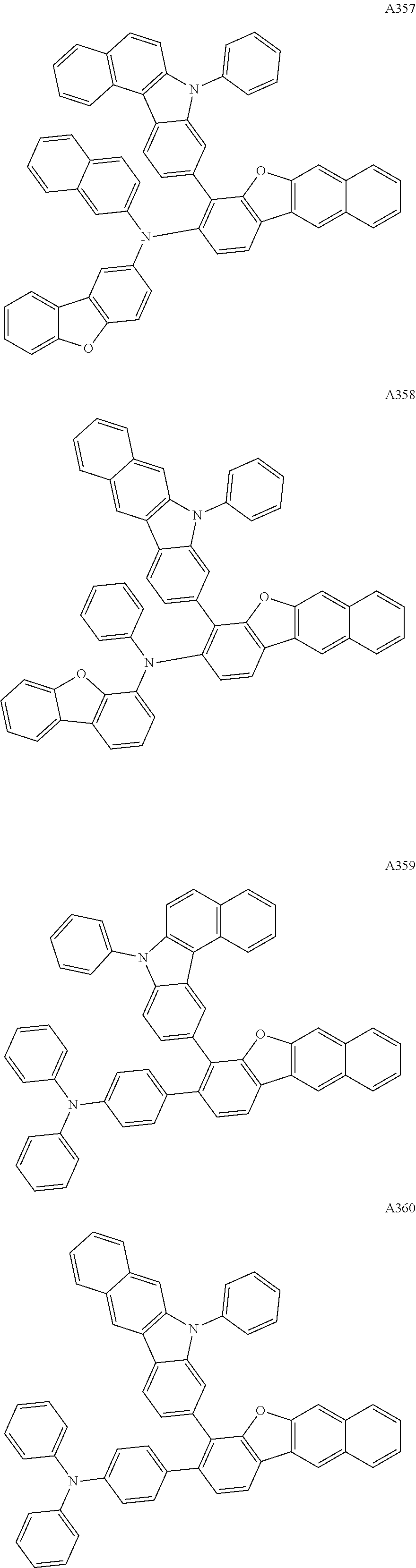 [SMILES: c1ccc(-n2c3cc(-c4c(N(c5ccc6ccccc6c5)c5ccc6oc7ccccc7c6c5)ccc5c4oc4cc6ccccc6cc45)ccc3c3c4ccccc4ccc32)cc1 ; c1ccc(N(c2ccc3c(oc4cc5ccccc5cc43)c2-c2ccc3c4cc5ccccc5cc4n(-c4ccccc4)c3c2)c2cccc3c2oc2ccccc23)cc1 ; c1ccc(N(c2ccccc2)c2ccc(-c3ccc4c(oc5cc6ccccc6cc54)c3-c3ccc4c(c3)c3c5ccccc5ccc3n4-c3ccccc3)cc2)cc1 ; c1ccc(N(c2ccccc2)c2ccc(-c3ccc4c(oc5cc6ccccc6cc54)c3-c3ccc4c5cc6ccccc6cc5n(-c5ccccc5)c4c3)cc2)cc1]

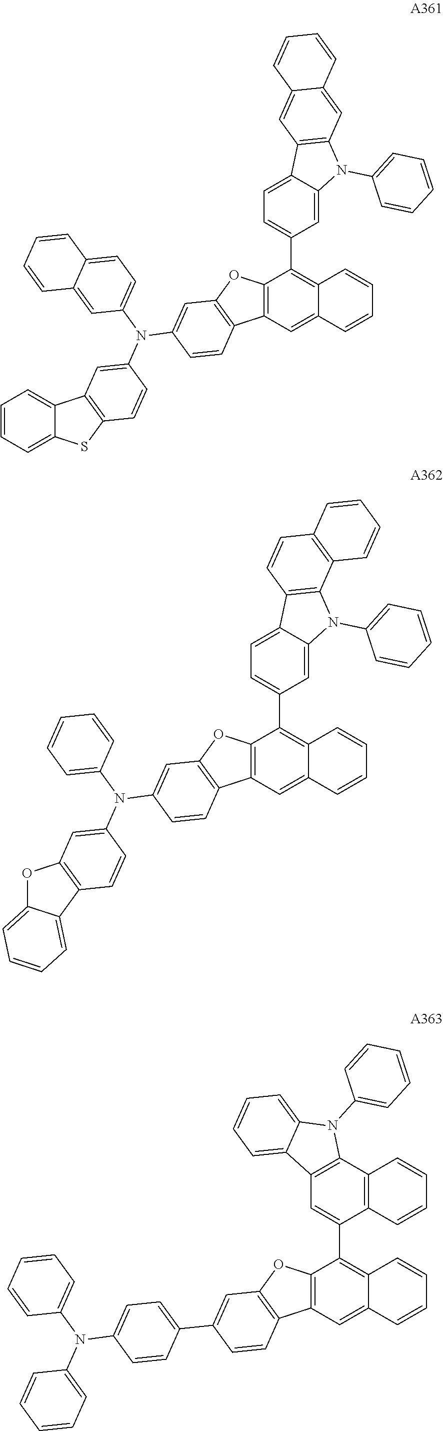 [SMILES: c1ccc(-n2c3cc(-c4c5ccccc5cc5c4oc4cc(N(c6ccc7ccccc7c6)c6ccc7sc8ccccc8c7c6)ccc45)ccc3c3cc4ccccc4cc32)cc1 ; c1ccc(N(c2ccc3c(c2)oc2ccccc23)c2ccc3c(c2)oc2c(-c4ccc5c6ccc7ccccc7c6n(-c6ccccc6)c5c4)c4ccccc4cc23)cc1 ; c1ccc(N(c2ccccc2)c2ccc(-c3ccc4c(c3)oc3c(-c5cc6c7ccccc7n(-c7ccccc7)c6c6ccccc56)c5ccccc5cc34)cc2)cc1]

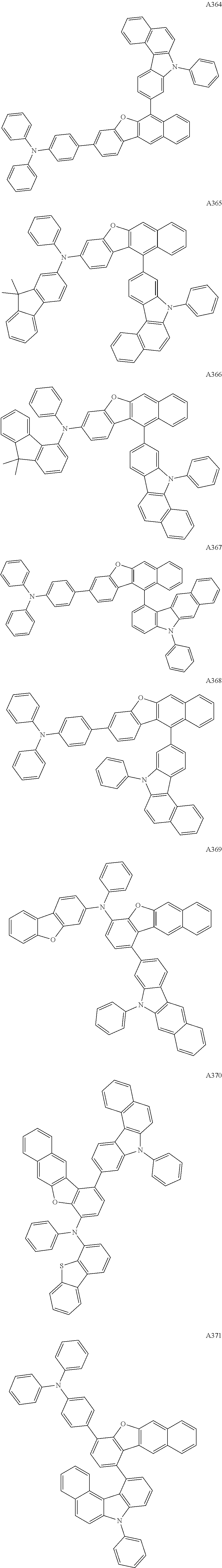

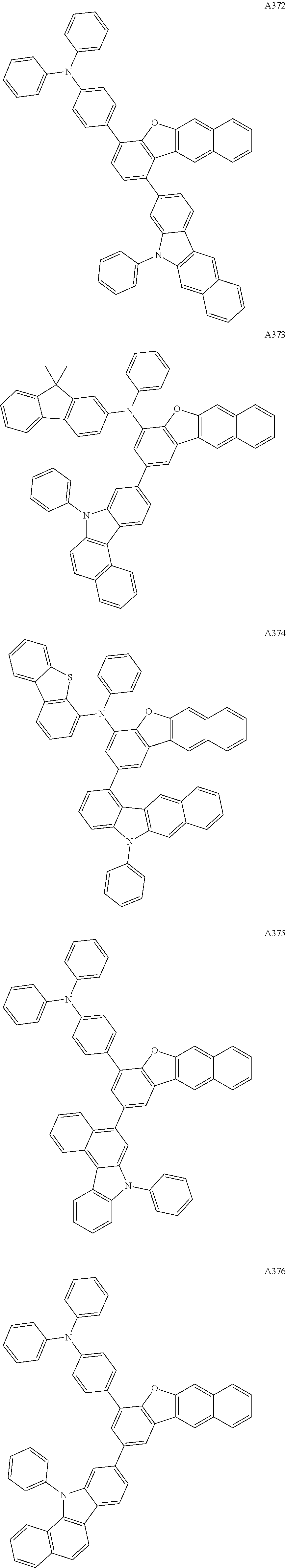 [SMILES: c1ccc(N(c2ccccc2)c2ccc(-c3ccc(-c4ccc5c6cc7ccccc7cc6n(-c6ccccc6)c5c4)c4c3oc3cc5ccccc5cc34)cc2)cc1 ; CC1(C)c2ccccc2-c2ccc(N(c3ccccc3)c3cc(-c4ccc5c6c7ccccc7ccc6n(-c6ccccc6)c5c4)cc4c3oc3cc5ccccc5cc34)cc21 ; c1ccc(N(c2cc(-c3cccc4c3c3cc5ccccc5cc3n4-c3ccccc3)cc3c2oc2cc4ccccc4cc23)c2cccc3c2sc2ccccc23)cc1 ; c1ccc(N(c2ccccc2)c2ccc(-c3cc(-c4cc5c(c6ccccc46)c4ccccc4n5-c4ccccc4)cc4c3oc3cc5ccccc5cc34)cc2)cc1 ; c1ccc(N(c2ccccc2)c2ccc(-c3cc(-c4ccc5c6ccc7ccccc7c6n(-c6ccccc6)c5c4)cc4c3oc3cc5ccccc5cc34)cc2)cc1]

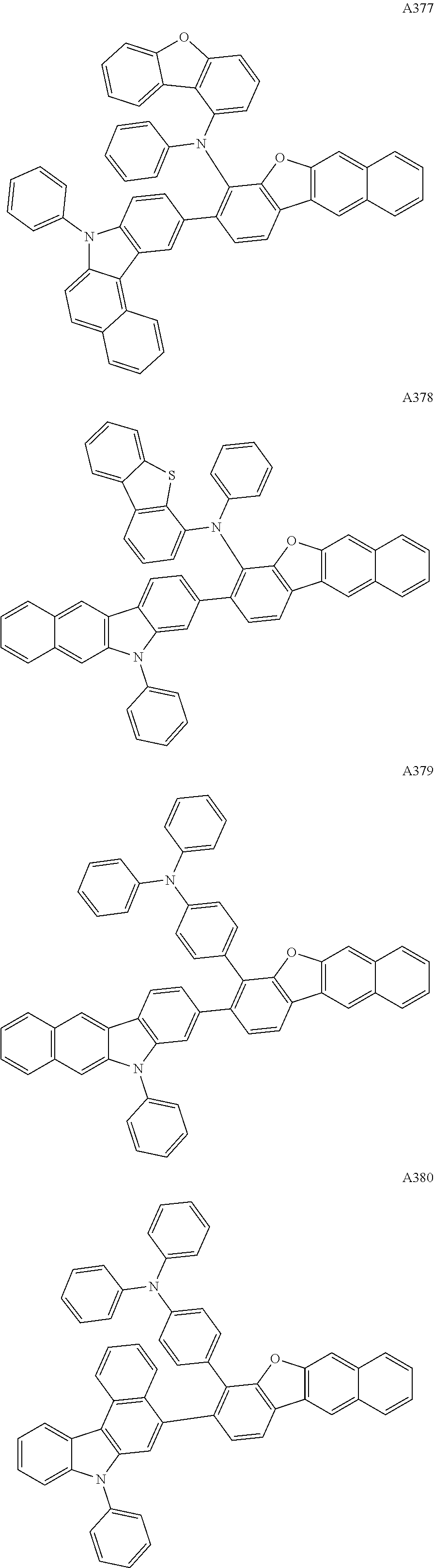 [SMILES: c1ccc(N(c2c(-c3ccc4c(c3)c3c5ccccc5ccc3n4-c3ccccc3)ccc3c2oc2cc4ccccc4cc23)c2cccc3oc4ccccc4c23)cc1 ; c1ccc(N(c2c(-c3ccc4c5cc6ccccc6cc5n(-c5ccccc5)c4c3)ccc3c2oc2cc4ccccc4cc23)c2cccc3c2sc2ccccc23)cc1 ; c1ccc(N(c2ccccc2)c2ccc(-c3c(-c4ccc5c6cc7ccccc7cc6n(-c6ccccc6)c5c4)ccc4c3oc3cc5ccccc5cc34)cc2)cc1 ; c1ccc(N(c2ccccc2)c2ccc(-c3c(-c4cc5c(c6ccccc46)c4ccccc4n5-c4ccccc4)ccc4c3oc3cc5ccccc5cc34)cc2)cc1]

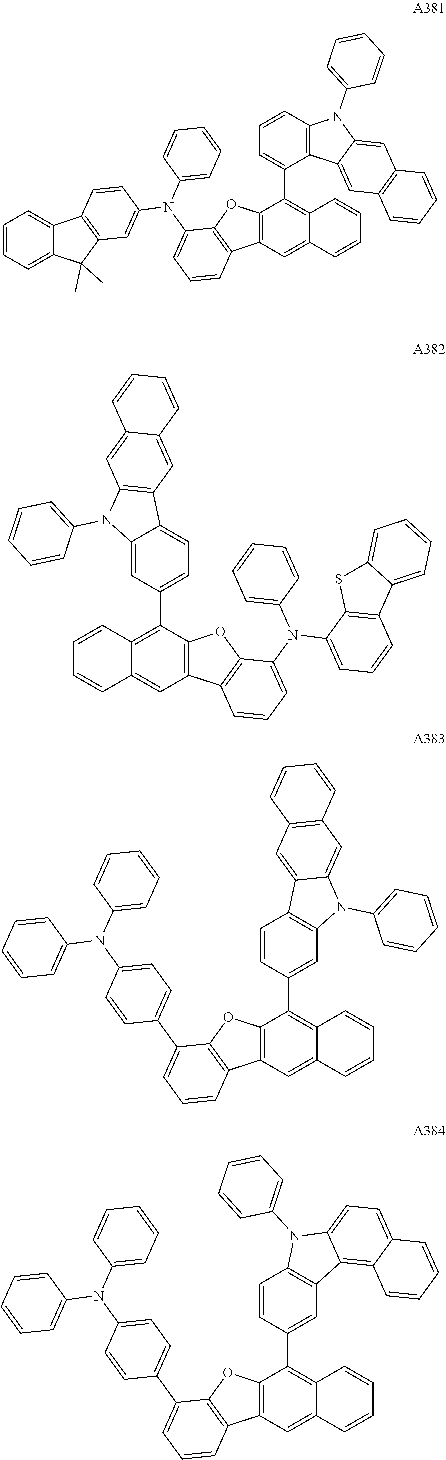 [SMILES: CC1(C)c2ccccc2-c2ccc(N(c3ccccc3)c3cccc4c3oc3c(-c5cccc6c5c5cc7ccccc7cc5n6-c5ccccc5)c5ccccc5cc34)cc21 ; c1ccc(N(c2cccc3c2oc2c(-c4ccc5c6cc7ccccc7cc6n(-c6ccccc6)c5c4)c4ccccc4cc23)c2cccc3c2sc2ccccc23)cc1 ; c1ccc(N(c2ccccc2)c2ccc(-c3cccc4c3oc3c(-c5ccc6c7cc8ccccc8cc7n(-c7ccccc7)c6c5)c5ccccc5cc34)cc2)cc1 ; c1ccc(N(c2ccccc2)c2ccc(-c3cccc4c3oc3c(-c5ccc6c(c5)c5c7ccccc7ccc5n6-c5ccccc5)c5ccccc5cc34)cc2)cc1]

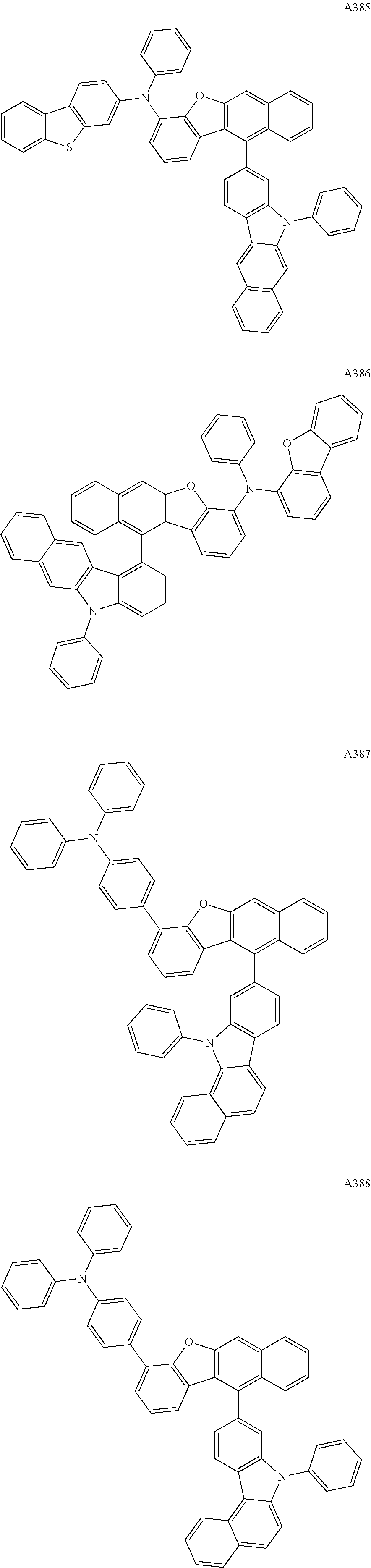 [SMILES: c1ccc(N(c2ccc3c(c2)sc2ccccc23)c2cccc3c2oc2cc4ccccc4c(-c4ccc5c6cc7ccccc7cc6n(-c6ccccc6)c5c4)c23)cc1 ; c1ccc(N(c2cccc3c2oc2ccccc23)c2cccc3c2oc2cc4ccccc4c(-c4cccc5c4c4cc6ccccc6cc4n5-c4ccccc4)c23)cc1 ; c1ccc(N(c2ccccc2)c2ccc(-c3cccc4c3oc3cc5ccccc5c(-c5ccc6c7ccc8ccccc8c7n(-c7ccccc7)c6c5)c34)cc2)cc1 ; c1ccc(N(c2ccccc2)c2ccc(-c3cccc4c3oc3cc5ccccc5c(-c5ccc6c7c8ccccc8ccc7n(-c7ccccc7)c6c5)c34)cc2)cc1]

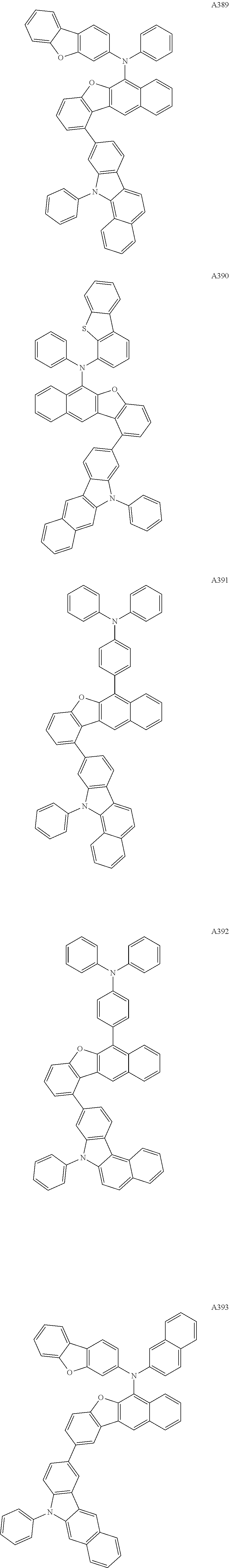

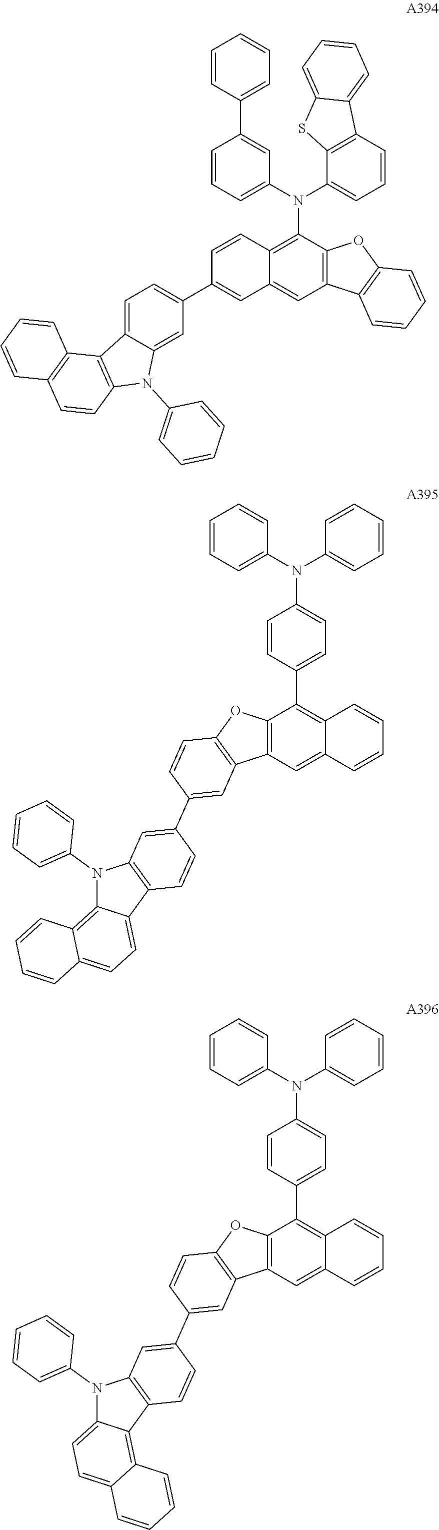 [SMILES: c1ccc(-c2cccc(N(c3c4ccc(-c5ccc6c7c8ccccc8ccc7n(-c7ccccc7)c6c5)cc4cc4c3oc3ccccc34)c3cccc4c3sc3ccccc34)c2)cc1 ; c1ccc(N(c2ccccc2)c2ccc(-c3c4ccccc4cc4c3oc3ccc(-c5ccc6c7ccc8ccccc8c7n(-c7ccccc7)c6c5)cc34)cc2)cc1 ; c1ccc(N(c2ccccc2)c2ccc(-c3c4ccccc4cc4c3oc3ccc(-c5ccc6c7c8ccccc8ccc7n(-c7ccccc7)c6c5)cc34)cc2)cc1]

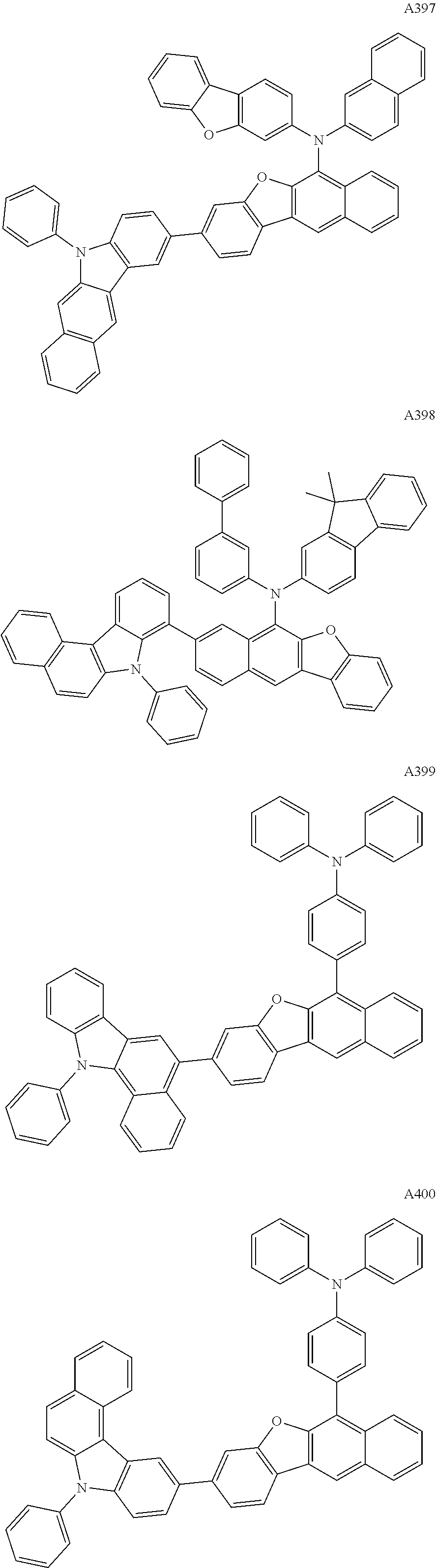 [SMILES: c1ccc(-n2c3ccc(-c4ccc5c(c4)oc4c(N(c6ccc7ccccc7c6)c6ccc7c(c6)oc6ccccc67)c6ccccc6cc45)cc3c3cc4ccccc4cc32)cc1 ; CC1(C)c2ccccc2-c2ccc(N(c3cccc(-c4ccccc4)c3)c3c4cc(-c5cccc6c7c8ccccc8ccc7n(-c7ccccc7)c56)ccc4cc4c3oc3ccccc34)cc21 ; c1ccc(N(c2ccccc2)c2ccc(-c3c4ccccc4cc4c3oc3cc(-c5cc6c7ccccc7n(-c7ccccc7)c6c6ccccc56)ccc34)cc2)cc1 ; c1ccc(N(c2ccccc2)c2ccc(-c3c4ccccc4cc4c3oc3cc(-c5ccc6c(c5)c5c7ccccc7ccc5n6-c5ccccc5)ccc34)cc2)cc1]

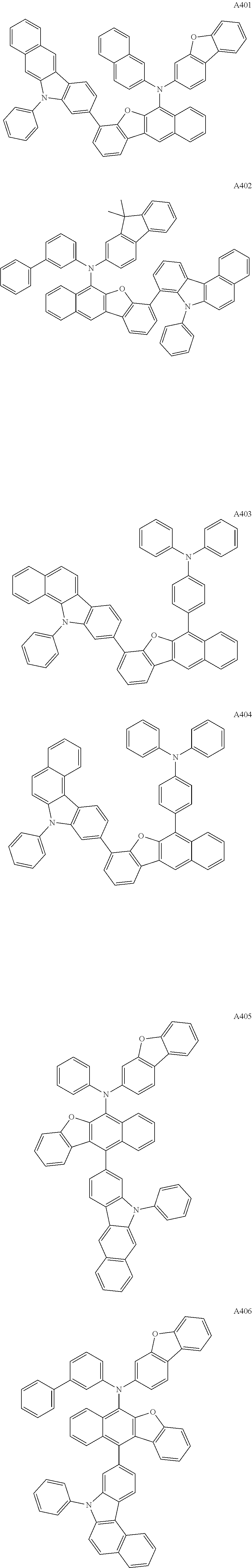

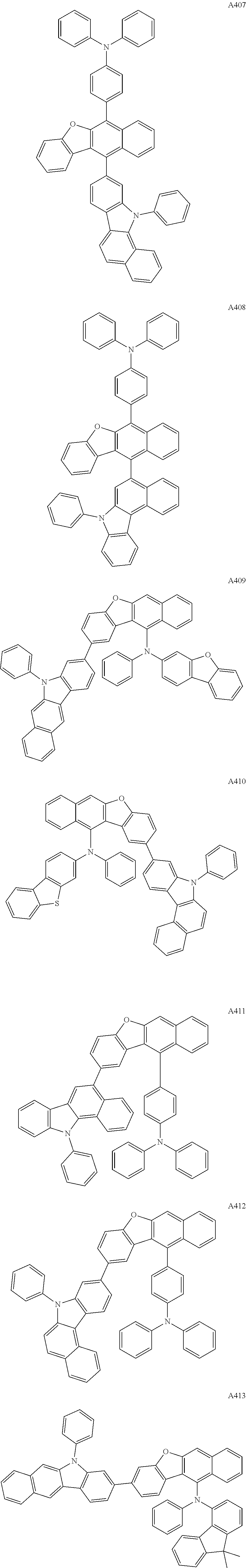

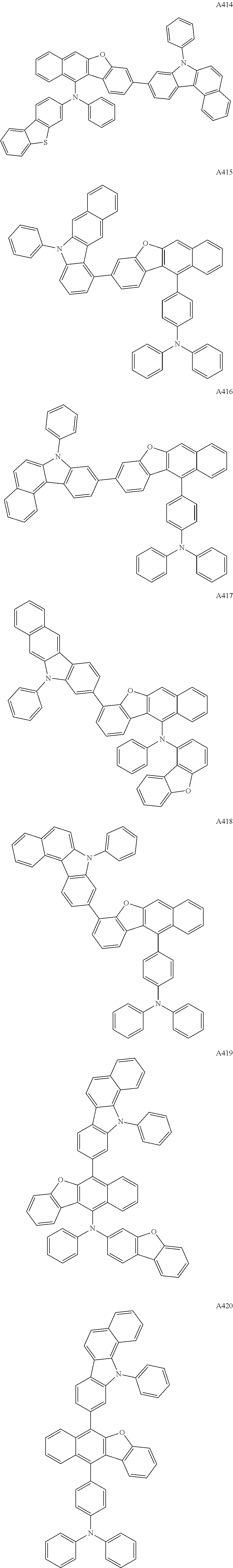

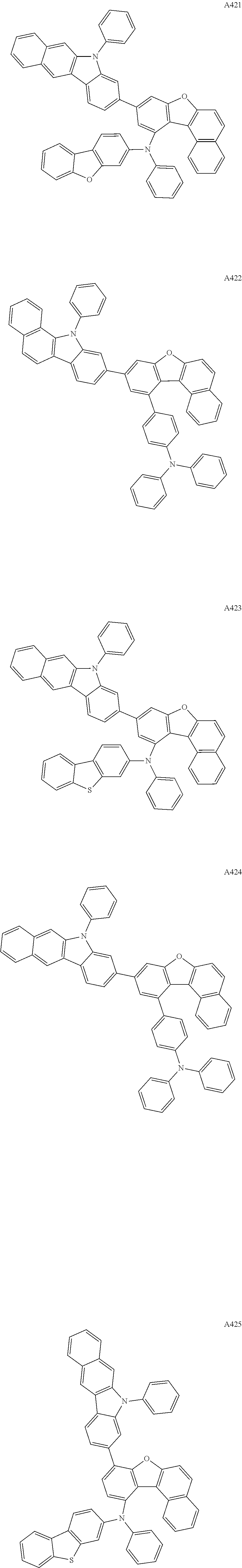 [SMILES: c1ccc(N(c2ccc3c(c2)oc2ccccc23)c2cc(-c3ccc4c5cc6ccccc6cc5n(-c5ccccc5)c4c3)cc3oc4ccc5ccccc5c4c23)cc1 ; c1ccc(N(c2ccccc2)c2ccc(-c3cc(-c4ccc5c6ccc7ccccc7c6n(-c6ccccc6)c5c4)cc4oc5ccc6ccccc6c5c34)cc2)cc1 ; c1ccc(N(c2ccc3c(c2)sc2ccccc23)c2cc(-c3ccc4c5cc6ccccc6cc5n(-c5ccccc5)c4c3)cc3oc4ccc5ccccc5c4c23)cc1 ; c1ccc(N(c2ccccc2)c2ccc(-c3cc(-c4ccc5c6cc7ccccc7cc6n(-c6ccccc6)c5c4)cc4oc5ccc6ccccc6c5c34)cc2)cc1 ; c1ccc(N(c2ccc3c(c2)sc2ccccc23)c2ccc(-c3ccc4c5cc6ccccc6cc5n(-c5ccccc5)c4c3)c3oc4ccc5ccccc5c4c23)cc1]

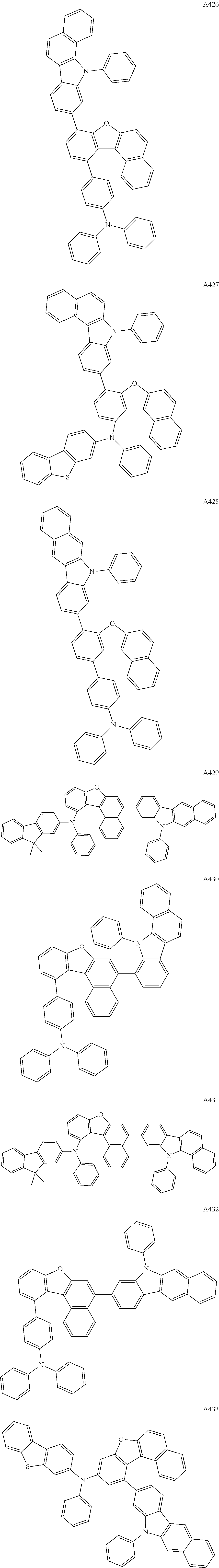

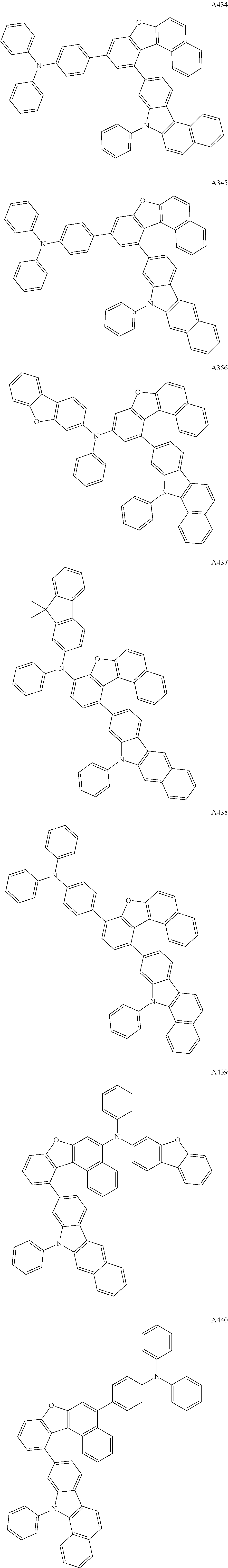

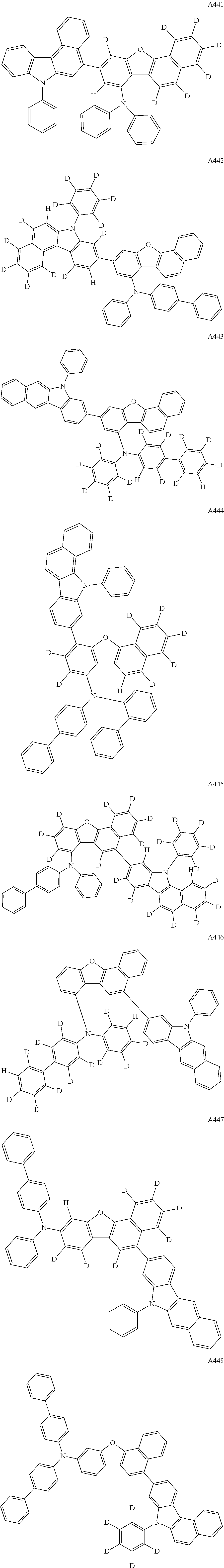

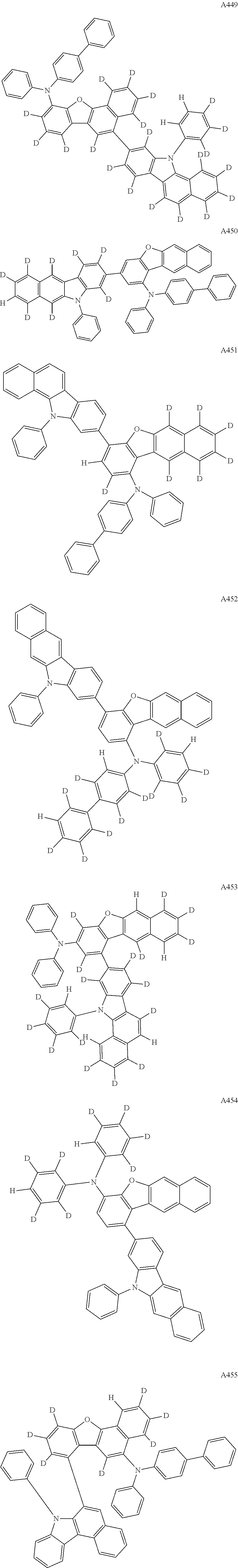

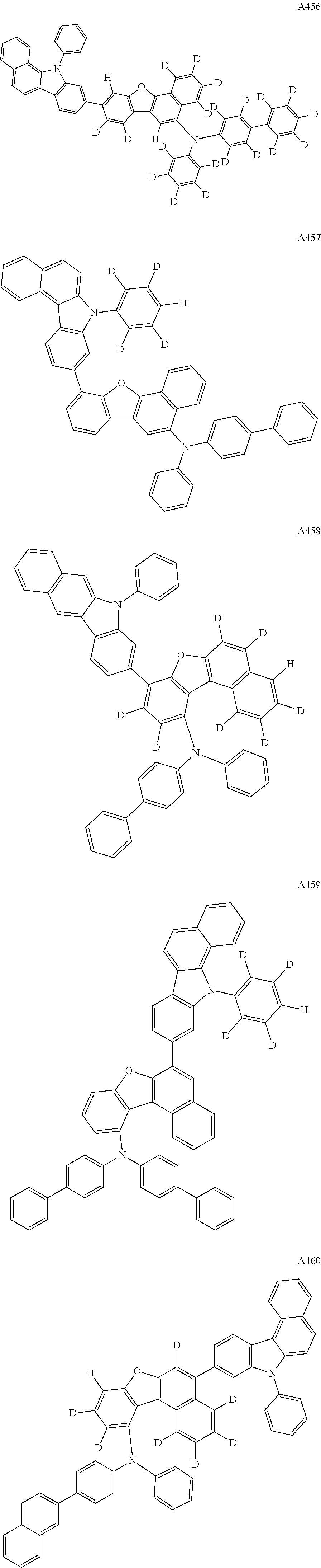

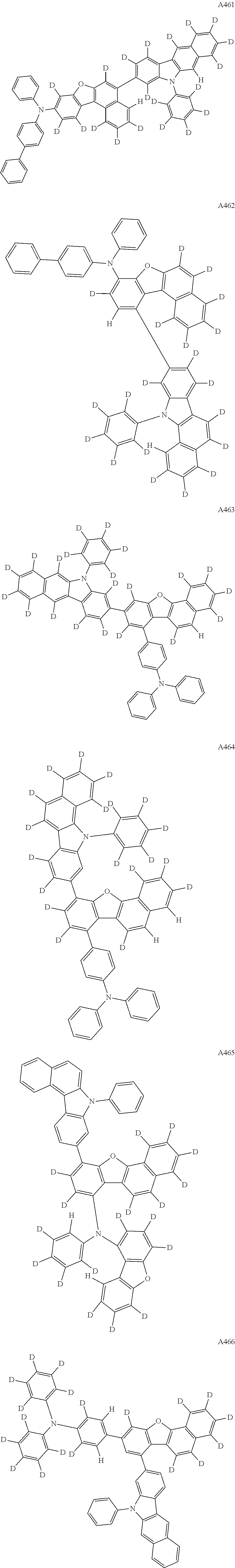

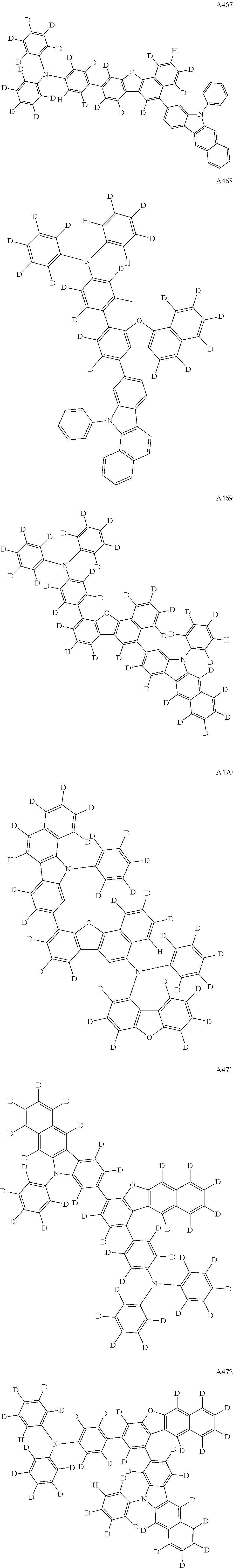

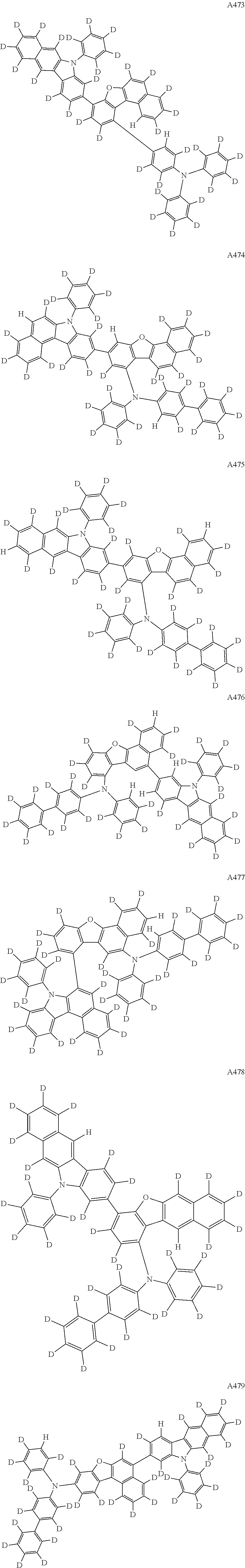

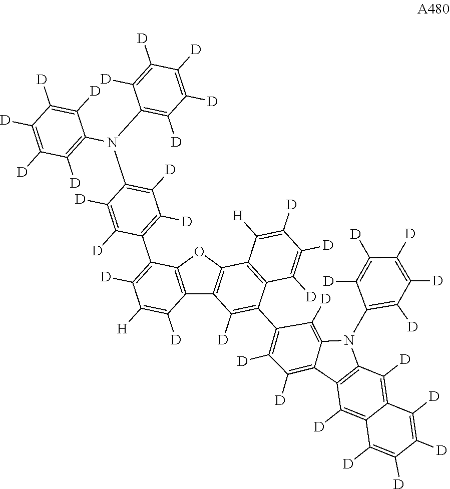 [SMILES: [2H]c1cc2c(c([2H])c1[2H])c(-c1c([2H])c([2H])c3c4c([2H])c5c([2H])c([2H])c([2H])c([2H])c5c([2H])c4n(-c4c([2H])c([2H])c([2H])c([2H])c4[2H])c3c1[2H])c([2H])c1c2oc2c(-c3c([2H])c([2H])c(N(c4c([2H])c([2H])c([2H])c([2H])c4[2H])c4c([2H])c([2H])c([2H])c([2H])c4[2H])c([2H])c3[2H])c([2H])cc([2H])c21]

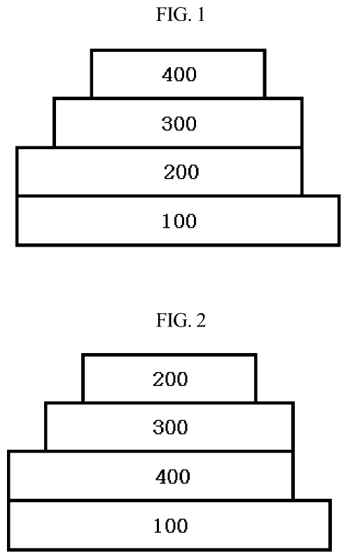

FIGS. 1 to 3 illustrate the stacking order of electrodes and organic layers of a light-emitting device according to an embodiment of the present application. However, the scope of the present application is not intended to be limited to these drawings, and the structure of organic light-emitting devices known in the art may also be applied to the present application.

Referring to FIG. 1, a light-emitting device is illustrated, in which a first electrode (anode, 200), an organic layer 300, and a second electrode (cathode, 400) are sequentially stacked on a substrate 100. However, it is not limited to this structure, and as shown in FIG. 2, an organic light-emitting device may be implemented in which the cathode 400, the organic layer 300, and the anode 200 are sequentially stacked on the substrate.

In an some embodiment, the first electrode may be an anode, and the second electrode may be a cathode. Alternatively, the first electrode may be a cathode, and the second electrode may be an anode.

According to some embodiments, materials with a relatively high work function may be used as the anode material, such as transparent conductive oxides, metals, or conductive polymers. Examples of the anode material may include metals such as vanadium, chromium, copper, zinc, and gold, or an alloy thereof; metal oxides such as zinc oxide, indium oxide, indium tin oxide (ITO), and indium zinc oxide (IZO); combinations of metals and oxides such as ZnO:Al or SnO₂:Sb; and conductive polymers such as poly(3-methylthiophene), poly[3,4-(ethylene-1,2-dioxy)thiophene] (PEDOT), polypyrrole and polyaniline, but are not limited thereto.

Materials with a relatively low work function may be used as cathode materials, such as metals, metal oxides, or conductive polymers. Examples of cathode materials may include metals such as magnesium, calcium, sodium, potassium, titanium, indium, yttrium, lithium, gadolinium, aluminum, silver, tin, and lead, or their alloys; and multilayer materials such as LiF/Al or LiO₂/Al, but are not limited thereto.

The light-emitting device may include one or more organic layers 300. Each of the plurality of organic layers 300 may independently have a single-layer or multi-layer structure.

In an some embodiment, at least one of the organic layers 300 may include a heterocyclic compound represented by Chemical Formula 1 above.

In some embodiments, at least one of the organic layers 300 may include two or more heterocyclic compounds represented by Chemical Formula 1 above.

As used herein, the specific details of the heterocyclic compound represented by Chemical Formula 1 are the same as those described above.

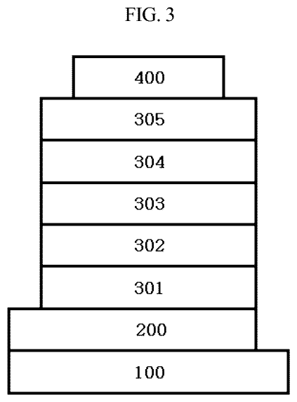

FIG. 3 is a cross-sectional view illustrating an exemplary light-emitting device in which the organic layer has a multi-layer structure.

The light-emitting device shown in FIG. 3 may include a hole injection layer 301, a hole transport layer 302, an emission layer 303, an electron transport layer 304, and an electron injection layer 305. However, the scope of the present application is not limited to this stacked structure, and layers other than the emission layer may be omitted, and other functional layers may be added.

The emission layer 303 may include a host material that is excited by holes and electrons, and a dopant material that increases luminous efficiency through energy absorption and emission.

In one embodiment, the emission layer 303 may be independently patterned for each red light-emitting device (Pr), green light-emitting device (Pg), and blue light-emitting device (Pb), thereby emitting different colors of light for each element. For example, the emission layer 303 may be patterned into a red emission layer, a green emission layer, and a blue emission layer for each element.

In one embodiment, the emission layer 303 may not be patterned for each light-emitting device, but may be provided in common to a plurality of light-emitting devices. For example, the emission layer 303 may emit white light, and the color of each element may be implemented through a color filter.

The host material may include a phosphorescent host, a fluorescent host, or a combination thereof. For example, the host material may include ADN (9,10-di(2-naphthyl) anthracene), MADN (2-methyl-9,10-bis(naphthalen-2-yl) anthracene), TBADN (9,10-di-(2-naphthyl)-2-t-butyl-anthracene), CBP (4,4′-bis(N-carbazolyl)-1,1′-biphenyl), mCP(1,3-di-9-carbazolylbenzene), TCP (1,3,5-tri (carbazol-9-yl)benzene), DPEPO (bis[2-(diphenylphosphino)phenyl]ether oxide), PPF (2,8-bis(diphenylphosphoryl)dibenzo[b,d]furan), TCTA (4,4′, 4″-tris(carbazol-9-yl)-triphenylamine), CP1 (hexaphenylcyclotriphosphazene), UGH2 (1,4-bis(triphenylsilyl)benzene), DPSiO₃ (hexaphenylcyclotrisiloxane), DPSiO₄ (octaphenylcyclotetrasiloxane), or a combination thereof.

The dopant material may include a phosphorescent dopant, a fluorescent dopant, or a combination thereof. For example, the dopant material may include a metal complex containing iridium (Ir), platinum (Pt), osmium (Os), gold (Au), titanium (Ti), zirconium (Zr), hafnium (Hf), europium (Eu), terbium (Tb), or thulium (Tm), or BCzVB (1,4-bis[2-(3-N-ethylcarbazoryl) vinyl]benzene), DPAVB (4-(di-p-tolylamino)-4′-[(di-p-tolylamino) styryl]stilbene), N-BDAVBi (N-(4-((E)-2-(6-((E)-4 (diphenylamino) styryl) naphthalen-2-yl) vinyl)phenyl)-N-phenylbenzenamine), DPAVBi (4,4′-bis[2-(4 (N,N-diphenylamino)phenyl) vinyl]biphenyl), TBP (2,5,8,11-tetra-t-butylperylene), or a combination thereof.

In one embodiment, the host material may include a heterocyclic compound represented by Chemical Formula 1 above. For example, the heterocyclic compound may serve as a red phosphorescent dopant.

By including the heterocyclic compound as the host material, the charge transport properties within the light-emitting device and the stability of the emission layer 303 may be further improved, thereby increasing the light-emitting efficiency and lifetime of the light-emitting device. Accordingly, the light-emitting properties may be improved without increasing the operating voltage.

In some embodiments, the content of the dopant material in the emission layer 303 may be about 0.01 parts by weight or more, about 1 part by weight or more, or about 2 parts by weight or more, and may be about 15 parts by weight or less, about 10 parts by weight or less, or about 8 parts by weight or less, based on 100 parts by weight of the host material. Within this range, the formation and emission energy of excitons may be increased, and the light-emitting efficiency and stability may be further improved.

In some embodiments, the organic layer 300 may further include a compound represented by Chemical Formula 4 below.

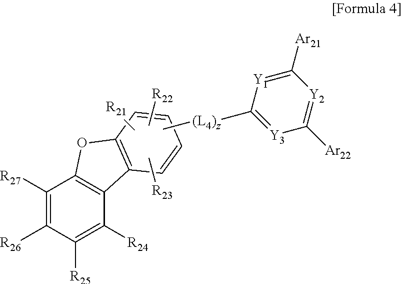

In Chemical Formula 4, any one of R₂₁ to R₂₇ may be a substituted or unsubstituted aryl group having 6 to 60 carbon atoms, or a substituted or unsubstituted heteroaryl group having 2 to 60 carbon atoms, and the remaining groups of R₂₁ to R₂₇ may each independently be selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a phosphine oxide group; and a silyl group; or two or more adjacent groups may be linked to form a substituted or unsubstituted aromatic hydrocarbon ring having 6 to 60 carbon atoms or a substituted or unsubstituted heterocycle having 2 to 60 carbon atoms.

Preferably, when any one of R₂₁ to R₂₇ is an aryl group or a heteroaryl group, the number of carbon atoms may be 50 or less, 40 or less, 30 or less, or 20 or less, and more preferably 12 or less.

L₄ may be a direct linkage, a substituted or unsubstituted arylene group having 6 to 60 carbon atoms, or a substituted or unsubstituted heteroarylene group having 2 to 60 carbon atoms.

In some embodiments, when L₄ is an arylene group or a heteroarylene group, the number of carbon atoms may be 50 or less, 40 or less, 30 or less, or 20 or less, and preferably 12 or less.

Preferably, L₄ may be a direct linkage or a phenylene group, and more preferably, a direct linkage. Accordingly, the lifetime characteristics and the light-emitting efficiency of the device may be further improved.

z may be an integer from 0 to 5. When z is 2 or more, a plurality of L₄ groups may be the same or different.

In one embodiment, b may be an integer from 1 to 3. In one embodiment, b may be 1 or 2.

Y₁ to Y₃ may each independently be CH, nitrogen (N), oxygen (O) or sulfur(S), and at least two of Y₁ to Y₃ may be nitrogen. Preferably, Y₁ to Y₃ may all be nitrogen.

Ar₂₁ and Ar₂₂ may each independently be a substituted or unsubstituted aryl group having 6 to 60 carbon atoms, a substituted or unsubstituted heteroaryl group having 2 to 60 carbon atoms, or a substituted or unsubstituted nitrogen-containing heterocycle having 2 to 60 carbon atoms.

Preferably, Ar₂₁ and Ar₂₂ may each have a number of carbon atoms of 50 or less, 40 or less, 30 or less, or 20 or less, and more preferably 12 or less.

Adjacent groups among R₂₄ to R₂₇ may be linked to form an aromatic ring. For example, the compound represented by Chemical Formula 4 above may include a compound represented by any one of Chemical Formulae 4-1 to 4-3 below.

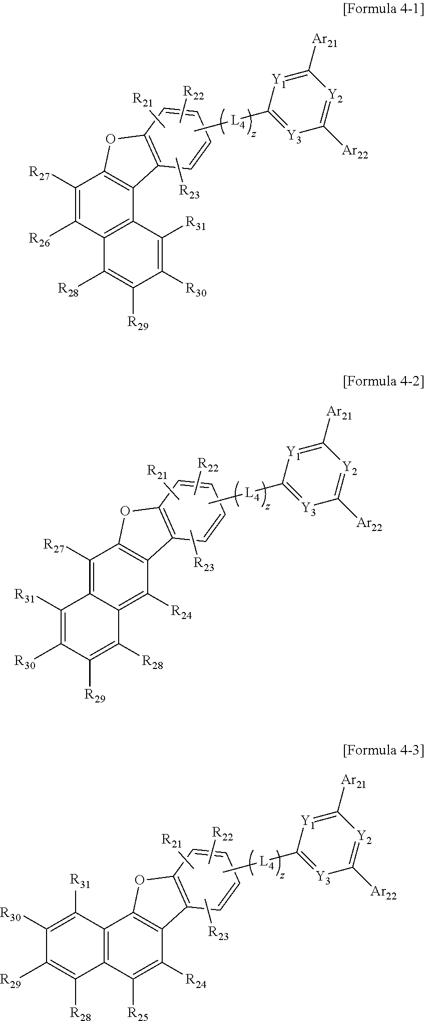

In Chemical Formulae 4-1 to 4-3, R₂₁ to R₂₇, Y₁ to Y₃, Ar₂₁, Ar₂₂, L₄ and z are the same as defined in Chemical Formula 4, and R₂₈ to R₃₁ may each independently be selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a phosphine oxide group; and a silyl group; or two or more adjacent groups may be linked to form a substituted or unsubstituted aromatic hydrocarbon ring having 6 to 60 carbon atoms or a substituted or unsubstituted heterocycle having 2 to 60 carbon atoms.

In Chemical Formula 4-1, R₂₆ and R₂₈ may be linked to form a substituted or unsubstituted aromatic hydrocarbon ring having 6 to 60 carbon atoms or a substituted or unsubstituted heterocycle having 2 to 60 carbon atoms.

In Chemical Formula 4-2, R₂₇ and R₃₁ may be linked to form a substituted or unsubstituted aromatic hydrocarbon ring having 6 to 60 carbon atoms or a substituted or unsubstituted heterocycle having 2 to 60 carbon atoms, and R₂₄ and R₂₈ may be linked to form a substituted or unsubstituted aromatic hydrocarbon ring having 6 to 60 carbon atoms or a substituted or unsubstituted heterocycle having 2 to 60 carbon atoms.

In Chemical Formula 4-3, R₂₅ and R₂₈ may be linked to form a substituted or unsubstituted aromatic hydrocarbon ring having 6 to 60 carbon atoms or a substituted or unsubstituted heterocycle having 2 to 60 carbon atoms.

In some embodiments, the deuterium content of the compound of Chemical Formula 4 may be 0%, or greater than 0 and not more than 100%.

In some embodiments, the deuterium content of the compound of Chemical Formula 4 may be 0%, or 5% to 100%.

In some embodiments, the deuterium content of the compound of Chemical Formula 4 may be 0%, or 10% to 100%.

In some embodiments, the deuterium content of the compound of Chemical Formula 4 may be 0%, or 15% to 100%.

According to some embodiments, the deuterium content of the compound of Chemical Formula 4 may be 0%, or may be between 20% and 100%.

According to some embodiments, the deuterium content of the compound of Chemical Formula 4 may be 0%, or 25% to 100%.

According to some embodiments, the deuterium content of the compound of Chemical Formula 4 may be 0%, or 30% to 100%.

According to some embodiments, the deuterium content of the compound of Chemical Formula 4 may be 0%, or 50% to 100%.

According to some embodiments, the deuterium content of the compound of Chemical Formula 4 may be 0%, or 70% to 100%.

According to some embodiments, the deuterium content of the compound of Chemical Formula 4 may be 0%, or 90% to 100%.

According to some embodiments, the deuterium content of the compound of Chemical Formula 4 may be 0%.

According to some embodiments, the deuterium content of the compound of Chemical Formula 4 may be from 50% to 100%.

According to some embodiments, the deuterium content of the compound of Chemical Formula 4 may be from 70% to 100%.

According to some embodiments, the deuterium content of the compound of Chemical Formula 4 may be 70% to 100%.

According to some embodiments, the deuterium content of the compound of Chemical Formula 4 may be 90% to 100%.

When the organic layer 300 includes the compound represented by Chemical Formula 4 together with the heterocyclic compound, the electron and hole transport characteristics of the light-emitting device may be further enhanced, and the light-emitting efficiency and device lifetime may be further improved.

In one embodiment, the compound represented by Chemical Formula 4 may be included in the same organic layer as the heterocyclic compound, and for example, may be included in the emission layer 303. For example, the heterocyclic compound represented by Chemical Formula 1 may be a p-type host material, and the compound represented by Chemical Formula 4 may be an n-type host material.

For example, the heterocyclic compound represented by Chemical Formula 1 may have high hole transport ability, thereby acting as a donor, and the compound represented by Chemical Formula 4 may have a high electron transport ability, thereby acting as an acceptor. When these compounds are used together, an exciplex phenomenon may occur, thereby improving charge balance within the device.

In one embodiment, the weight ratio of the heterocyclic compound to the compound represented by Chemical Formula 4 in the organic layer 300 or the emission layer 303 may be 1:10 to 10:1, 1:8 to 8:1, 1:5 to 5:1, or 1:2 to 2:1. Within this range, both electron transport characteristics and hole transport characteristics in the organic layer 300 may be improved. Accordingly, the operating voltage of the light-emitting device may be lowered, and the light-emitting efficiency and lifetime characteristics may be further improved.

In some embodiments, the compound represented by Chemical Formula 4 above may include one or more compounds represented by any one of Chemical Formulae below.

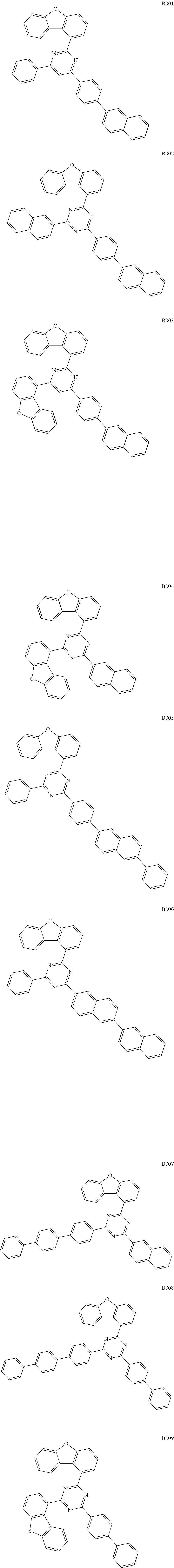

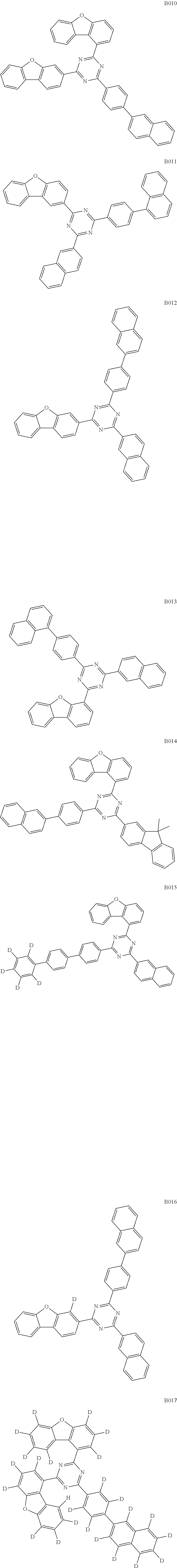

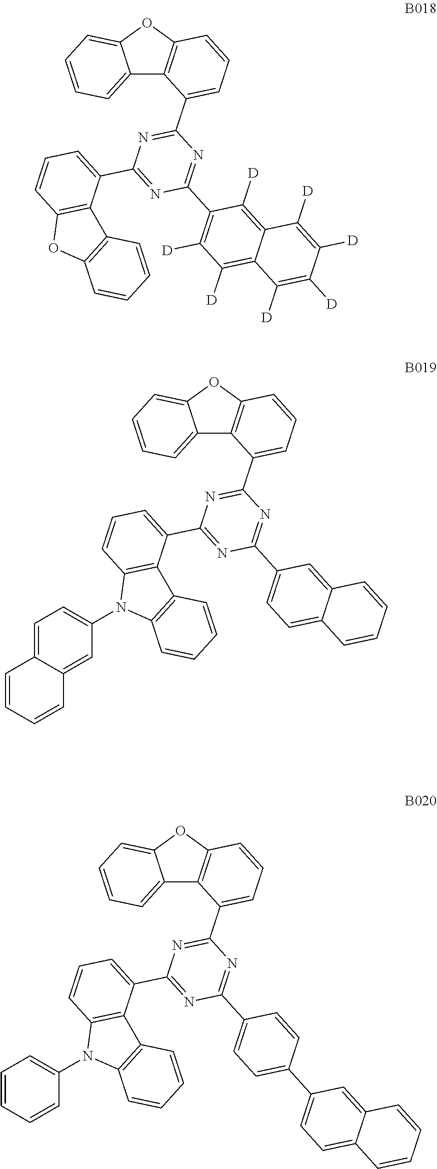 [SMILES: [2H]c1c([2H])c([2H])c2c([2H])c(-c3nc(-c4cccc5oc6ccccc6c45)nc(-c4cccc5oc6ccccc6c45)n3)c([2H])c([2H])c2c1[2H] ; c1ccc2cc(-c3nc(-c4cccc5oc6ccccc6c45)nc(-c4cccc5c4c4ccccc4n5-c4ccc5ccccc5c4)n3)ccc2c1 ; c1ccc(-n2c3ccccc3c3c(-c4nc(-c5ccc(-c6ccc7ccccc7c6)cc5)nc(-c5cccc6oc7ccccc7c56)n4)cccc32)cc1]

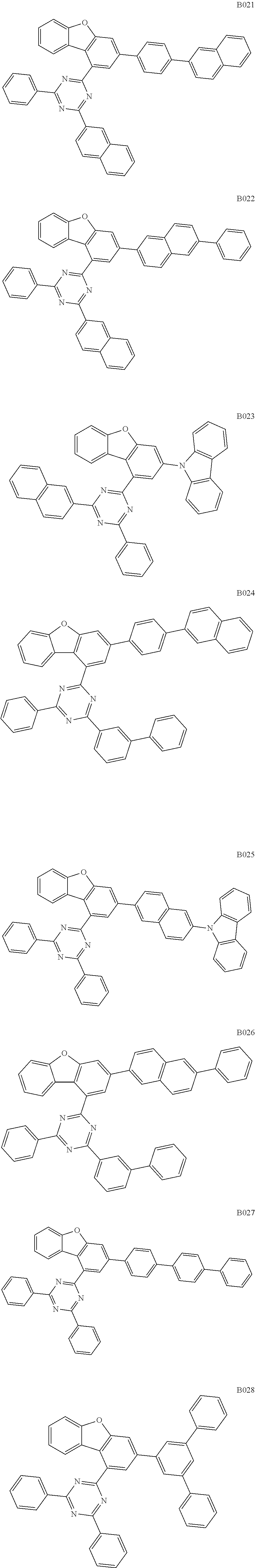

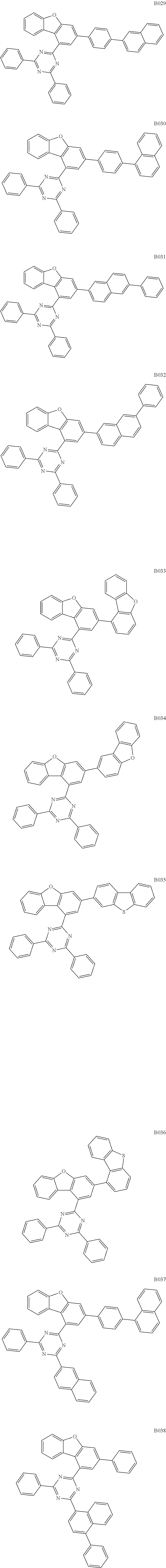

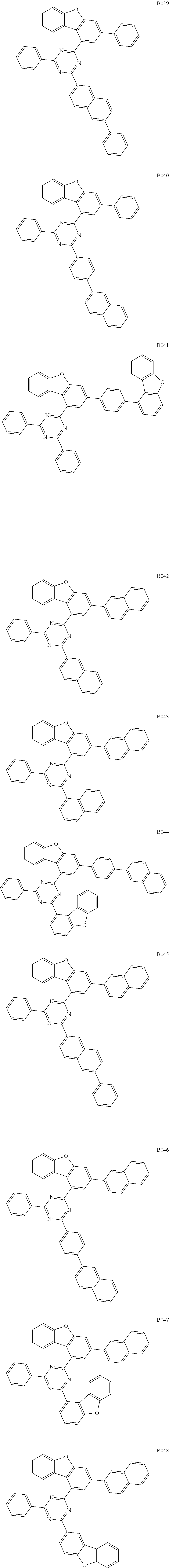

[chemical structure]

[chemical structure]

[chemical structure]

[chemical structure]

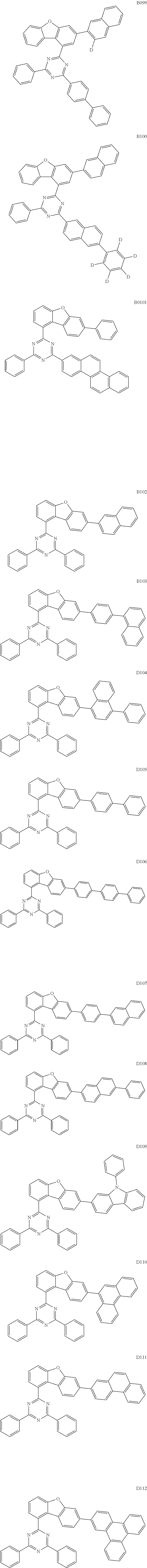

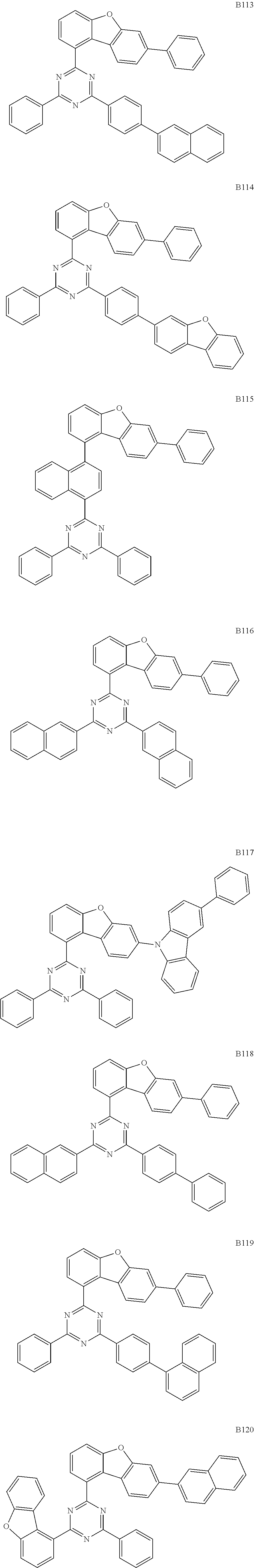

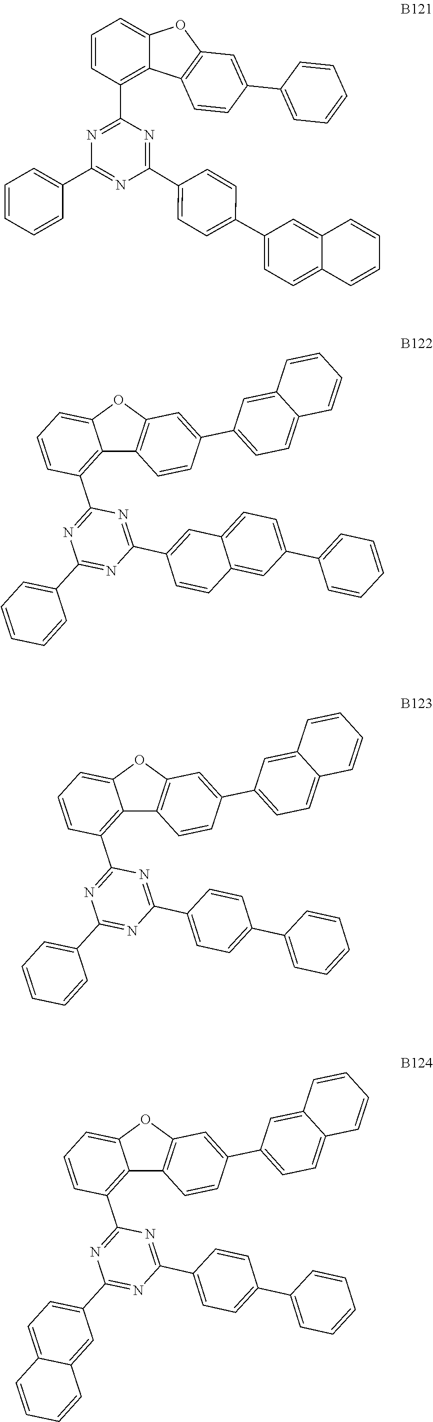 [SMILES: c1ccc(-c2ccc3c(c2)oc2cccc(-c4nc(-c5ccccc5)nc(-c5ccc(-c6ccc7ccccc7c6)cc5)n4)c23)cc1 ; c1ccc(-c2ccc3cc(-c4nc(-c5ccccc5)nc(-c5cccc6oc7cc(-c8ccc9ccccc9c8)ccc7c56)n4)ccc3c2)cc1 ; c1ccc(-c2ccc(-c3nc(-c4ccccc4)nc(-c4cccc5oc6cc(-c7ccc8ccccc8c7)ccc6c45)n3)cc2)cc1 ; c1ccc(-c2ccc(-c3nc(-c4ccc5ccccc5c4)nc(-c4cccc5oc6cc(-c7ccc8ccccc8c7)ccc6c45)n3)cc2)cc1]

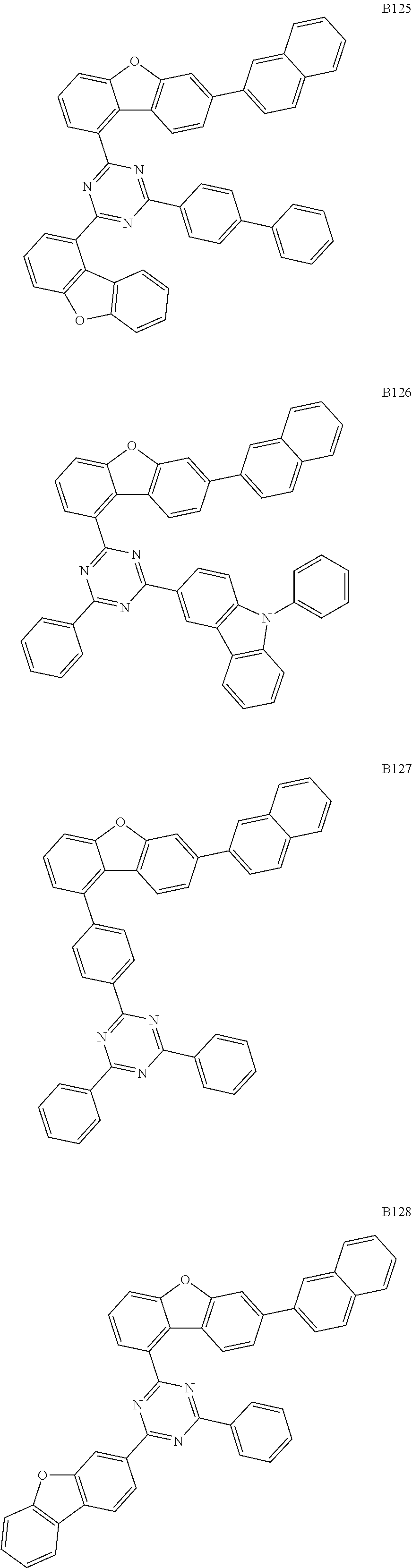 [SMILES: c1ccc(-c2ccc(-c3nc(-c4cccc5oc6ccccc6c45)nc(-c4cccc5oc6cc(-c7ccc8ccccc8c7)ccc6c45)n3)cc2)cc1 ; c1ccc(-c2nc(-c3ccc4c(c3)c3ccccc3n4-c3ccccc3)nc(-c3cccc4oc5cc(-c6ccc7ccccc7c6)ccc5c34)n2)cc1 ; c1ccc(-c2nc(-c3ccccc3)nc(-c3ccc(-c4cccc5oc6cc(-c7ccc8ccccc8c7)ccc6c45)cc3)n2)cc1 ; c1ccc(-c2nc(-c3ccc4c(c3)oc3ccccc34)nc(-c3cccc4oc5cc(-c6ccc7ccccc7c6)ccc5c34)n2)cc1]

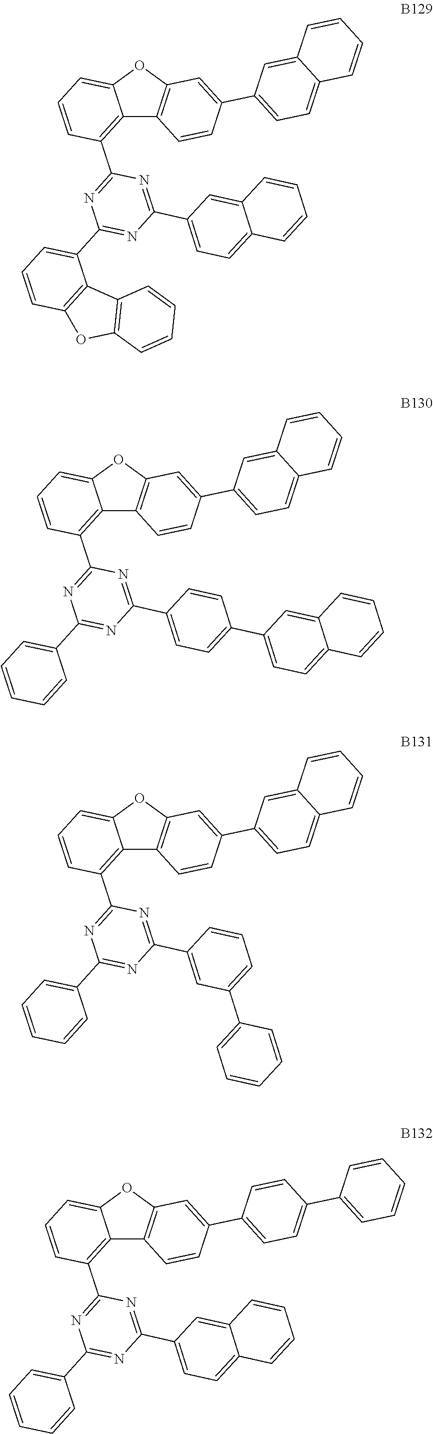 [SMILES: c1ccc2cc(-c3ccc4c(c3)oc3cccc(-c5nc(-c6ccc7ccccc7c6)nc(-c6cccc7oc8ccccc8c67)n5)c34)ccc2c1 ; c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2cccc(-c3nc(-c4ccccc4)nc(-c4cccc5oc6cc(-c7ccc8ccccc8c7)ccc6c45)n3)c2)cc1 ; c1ccc(-c2ccc(-c3ccc4c(c3)oc3cccc(-c5nc(-c6ccccc6)nc(-c6ccc7ccccc7c6)n5)c34)cc2)cc1]

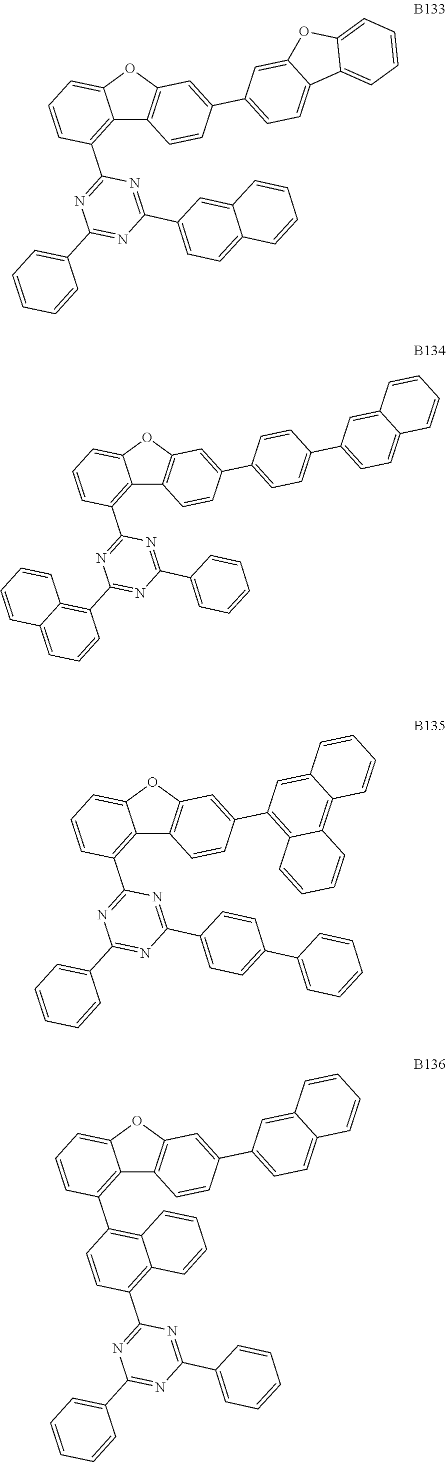 [SMILES: c1ccc(-c2nc(-c3ccc4ccccc4c3)nc(-c3cccc4oc5cc(-c6ccc7c(c6)oc6ccccc67)ccc5c34)n2)cc1 ; c1ccc(-c2nc(-c3cccc4ccccc34)nc(-c3cccc4oc5cc(-c6ccc(-c7ccc8ccccc8c7)cc6)ccc5c34)n2)cc1 ; c1ccc(-c2ccc(-c3nc(-c4ccccc4)nc(-c4cccc5oc6cc(-c7cc8ccccc8c8ccccc78)ccc6c45)n3)cc2)cc1 ; c1ccc(-c2nc(-c3ccccc3)nc(-c3ccc(-c4cccc5oc6cc(-c7ccc8ccccc8c7)ccc6c45)c4ccccc34)n2)cc1]

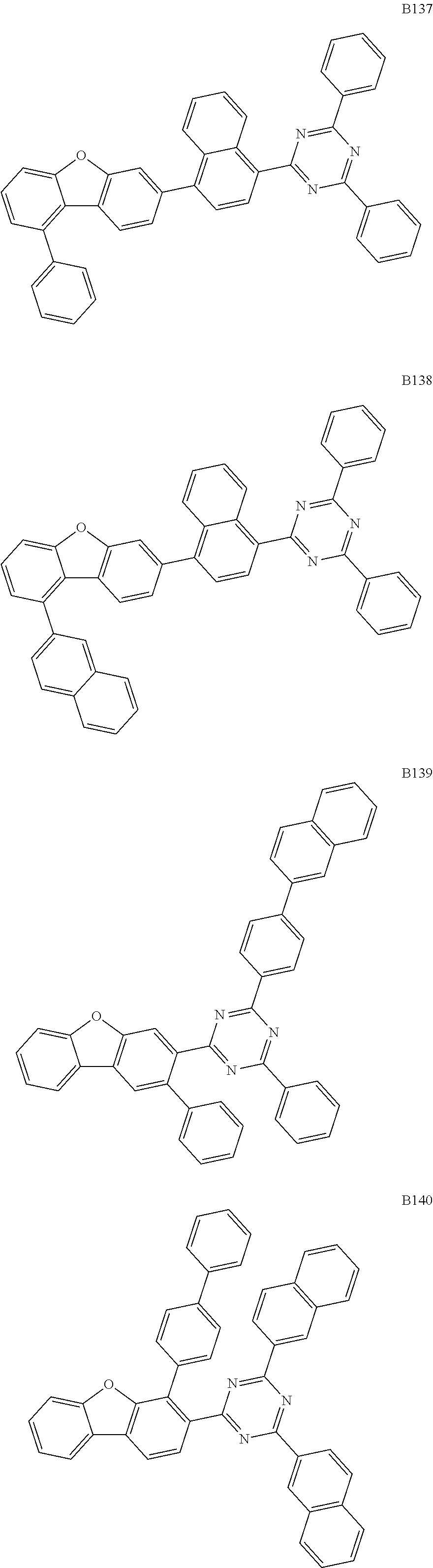 [SMILES: c1ccc(-c2nc(-c3ccccc3)nc(-c3ccc(-c4ccc5c(c4)oc4cccc(-c6ccccc6)c45)c4ccccc34)n2)cc1 ; c1ccc(-c2nc(-c3ccccc3)nc(-c3ccc(-c4ccc5c(c4)oc4cccc(-c6ccc7ccccc7c6)c45)c4ccccc34)n2)cc1 ; c1ccc(-c2nc(-c3ccc(-c4ccc5ccccc5c4)cc3)nc(-c3cc4oc5ccccc5c4cc3-c3ccccc3)n2)cc1 ; c1ccc(-c2ccc(-c3c(-c4nc(-c5ccc6ccccc6c5)nc(-c5ccc6ccccc6c5)n4)ccc4c3oc3ccccc34)cc2)cc1]

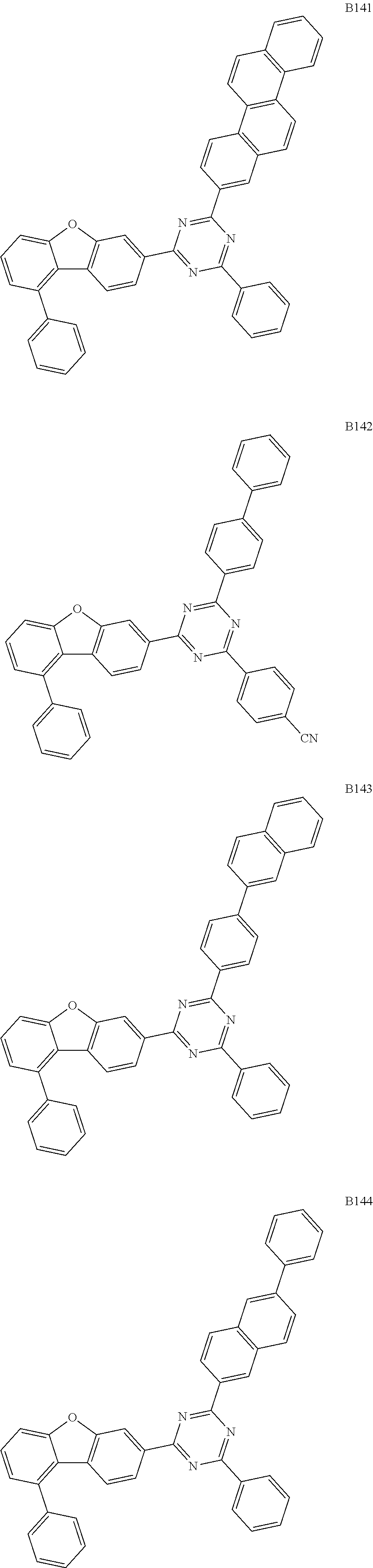 [SMILES: c1ccc(-c2nc(-c3ccc4c(ccc5c6ccccc6ccc45)c3)nc(-c3ccc4c(c3)oc3cccc(-c5ccccc5)c34)n2)cc1 ; N#Cc1ccc(-c2nc(-c3ccc(-c4ccccc4)cc3)nc(-c3ccc4c(c3)oc3cccc(-c5ccccc5)c34)n2)cc1 ; c1ccc(-c2nc(-c3ccc(-c4ccc5ccccc5c4)cc3)nc(-c3ccc4c(c3)oc3cccc(-c5ccccc5)c34)n2)cc1 ; c1ccc(-c2ccc3cc(-c4nc(-c5ccccc5)nc(-c5ccc6c(c5)oc5cccc(-c7ccccc7)c56)n4)ccc3c2)cc1]

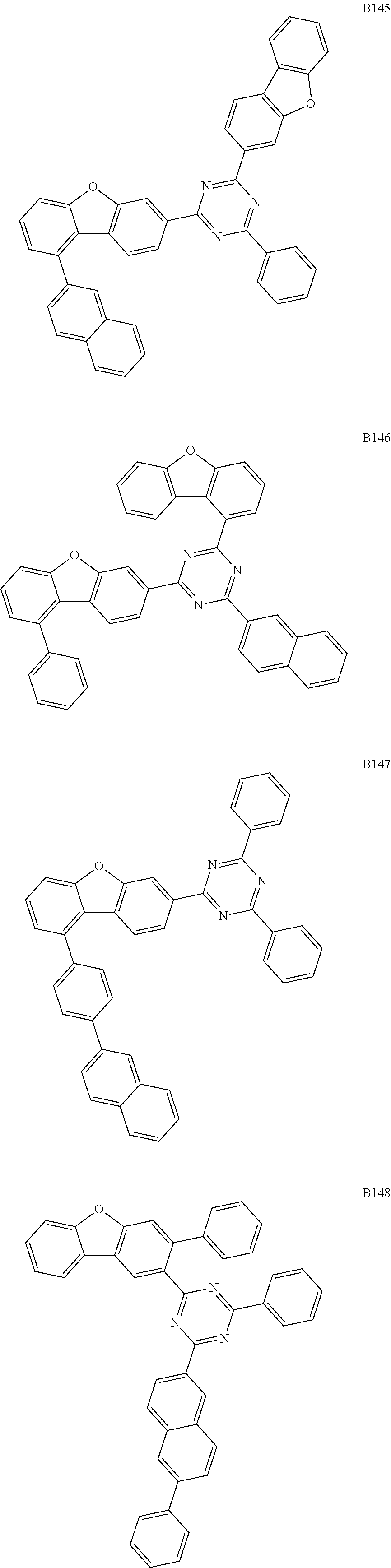 [SMILES: c1ccc(-c2nc(-c3ccc4c(c3)oc3ccccc34)nc(-c3ccc4c(c3)oc3cccc(-c5ccc6ccccc6c5)c34)n2)cc1 ; c1ccc(-c2cccc3oc4cc(-c5nc(-c6ccc7ccccc7c6)nc(-c6cccc7oc8ccccc8c67)n5)ccc4c23)cc1 ; c1ccc(-c2nc(-c3ccccc3)nc(-c3ccc4c(c3)oc3cccc(-c5ccc(-c6ccc7ccccc7c6)cc5)c34)n2)cc1 ; c1ccc(-c2ccc3cc(-c4nc(-c5ccccc5)nc(-c5cc6c(cc5-c5ccccc5)oc5ccccc56)n4)ccc3c2)cc1]

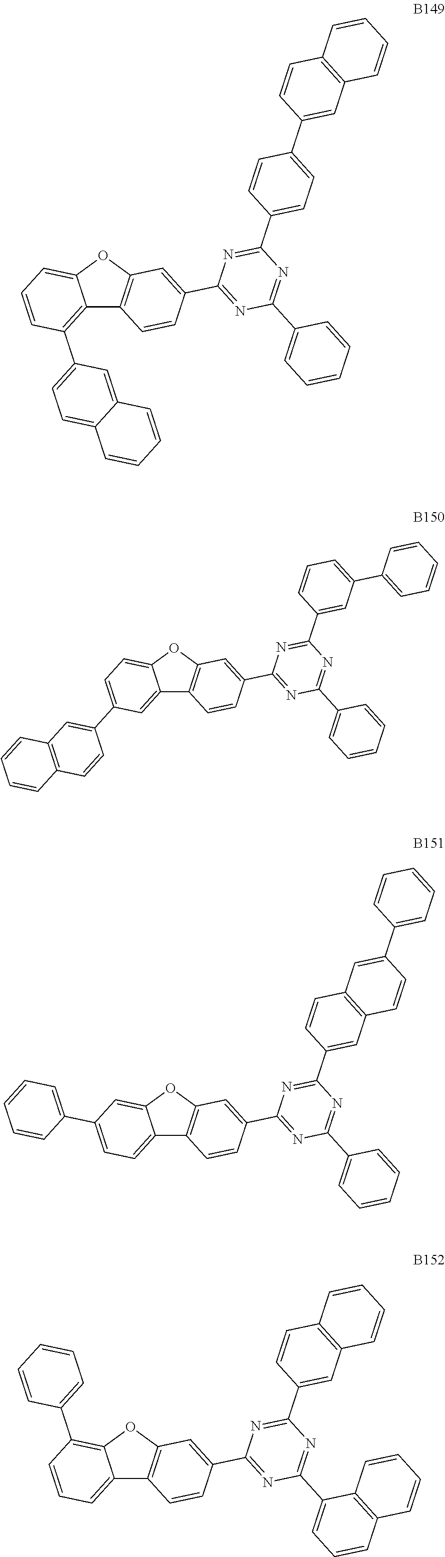 [SMILES: c1ccc(-c2nc(-c3ccc(-c4ccc5ccccc5c4)cc3)nc(-c3ccc4c(c3)oc3cccc(-c5ccc6ccccc6c5)c34)n2)cc1 ; c1ccc(-c2cccc(-c3nc(-c4ccccc4)nc(-c4ccc5c(c4)oc4ccc(-c6ccc7ccccc7c6)cc45)n3)c2)cc1 ; c1ccc(-c2ccc3cc(-c4nc(-c5ccccc5)nc(-c5ccc6c(c5)oc5cc(-c7ccccc7)ccc56)n4)ccc3c2)cc1 ; c1ccc(-c2cccc3c2oc2cc(-c4nc(-c5ccc6ccccc6c5)nc(-c5cccc6ccccc56)n4)ccc23)cc1]

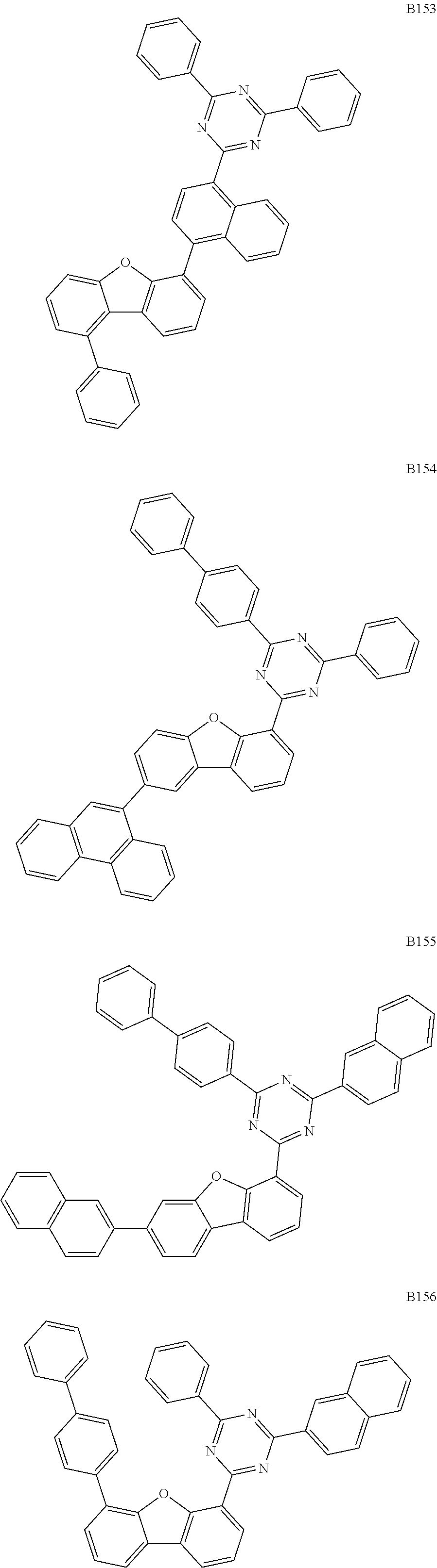 [SMILES: c1ccc(-c2nc(-c3ccccc3)nc(-c3ccc(-c4cccc5c4oc4cccc(-c6ccccc6)c45)c4ccccc34)n2)cc1 ; c1ccc(-c2ccc(-c3nc(-c4ccccc4)nc(-c4cccc5c4oc4ccc(-c6cc7ccccc7c7ccccc67)cc45)n3)cc2)cc1 ; c1ccc(-c2ccc(-c3nc(-c4ccc5ccccc5c4)nc(-c4cccc5c4oc4cc(-c6ccc7ccccc7c6)ccc45)n3)cc2)cc1 ; c1ccc(-c2ccc(-c3cccc4c3oc3c(-c5nc(-c6ccccc6)nc(-c6ccc7ccccc7c6)n5)cccc34)cc2)cc1]

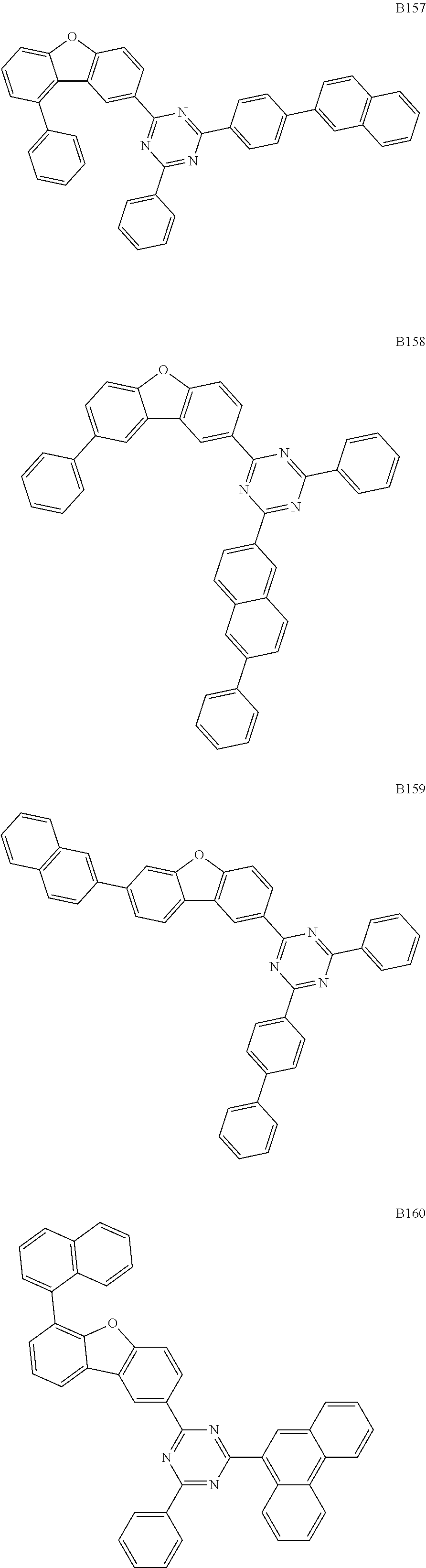 [SMILES: c1ccc(-c2nc(-c3ccc(-c4ccc5ccccc5c4)cc3)nc(-c3ccc4oc5cccc(-c6ccccc6)c5c4c3)n2)cc1 ; c1ccc(-c2ccc3cc(-c4nc(-c5ccccc5)nc(-c5ccc6oc7ccc(-c8ccccc8)cc7c6c5)n4)ccc3c2)cc1 ; c1ccc(-c2ccc(-c3nc(-c4ccccc4)nc(-c4ccc5oc6cc(-c7ccc8ccccc8c7)ccc6c5c4)n3)cc2)cc1 ; c1ccc(-c2nc(-c3ccc4oc5c(-c6cccc7ccccc67)cccc5c4c3)nc(-c3cc4ccccc4c4ccccc34)n2)cc1]

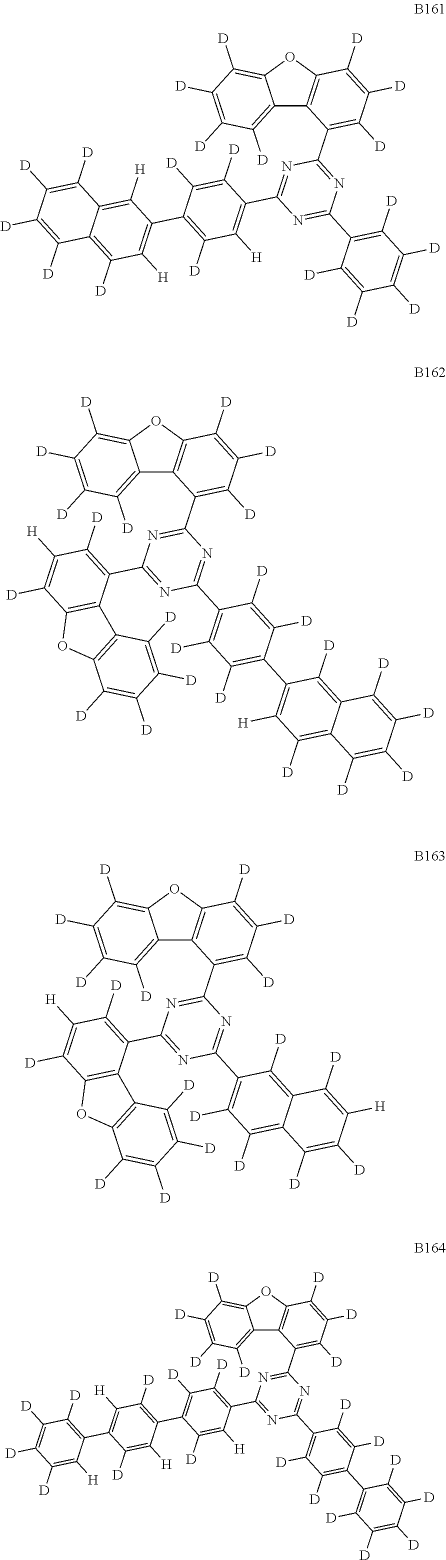

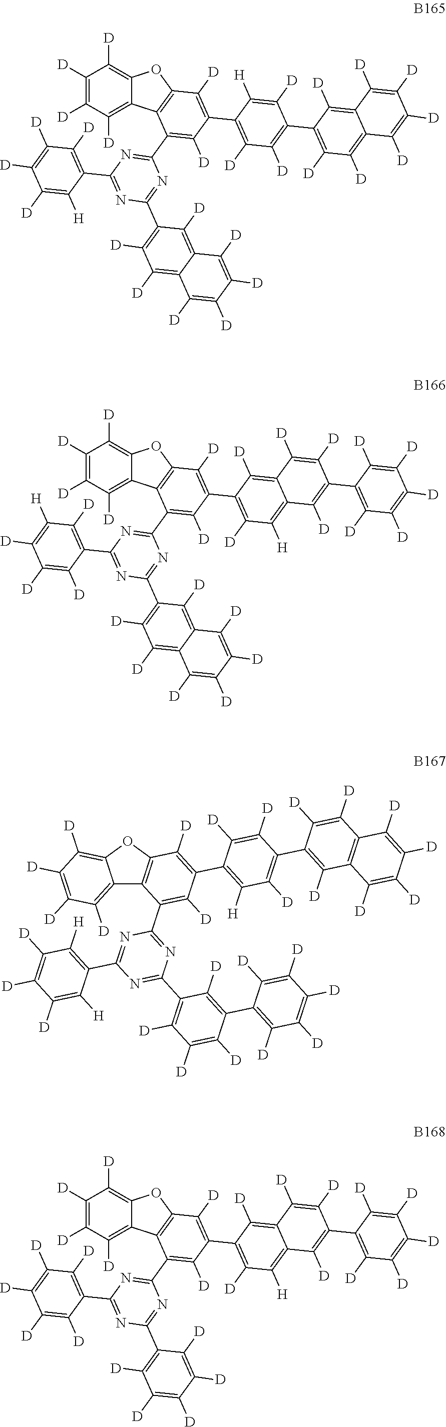

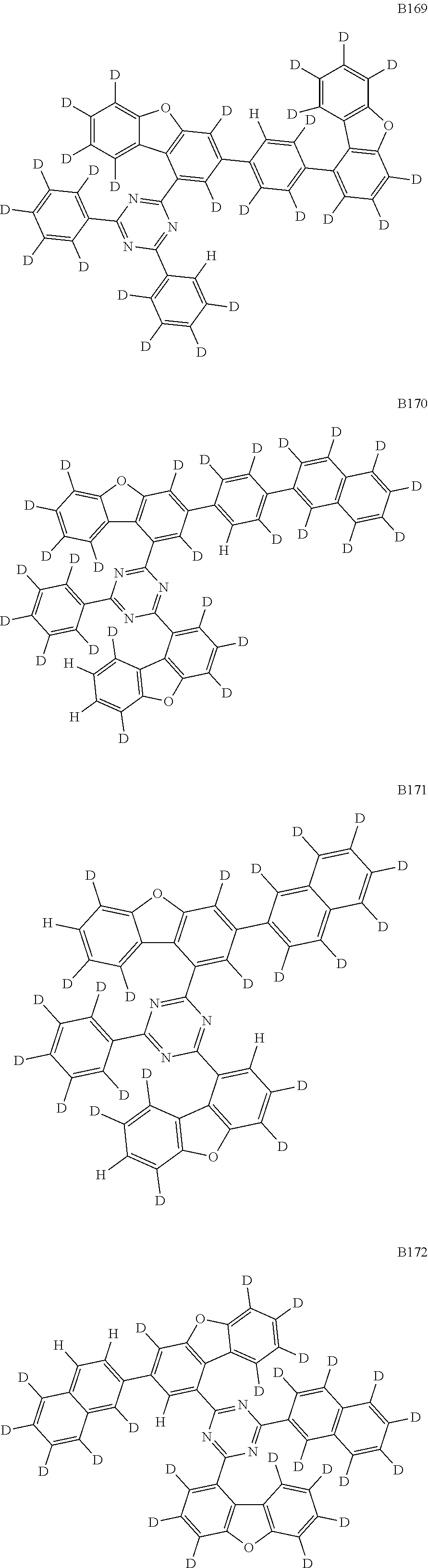

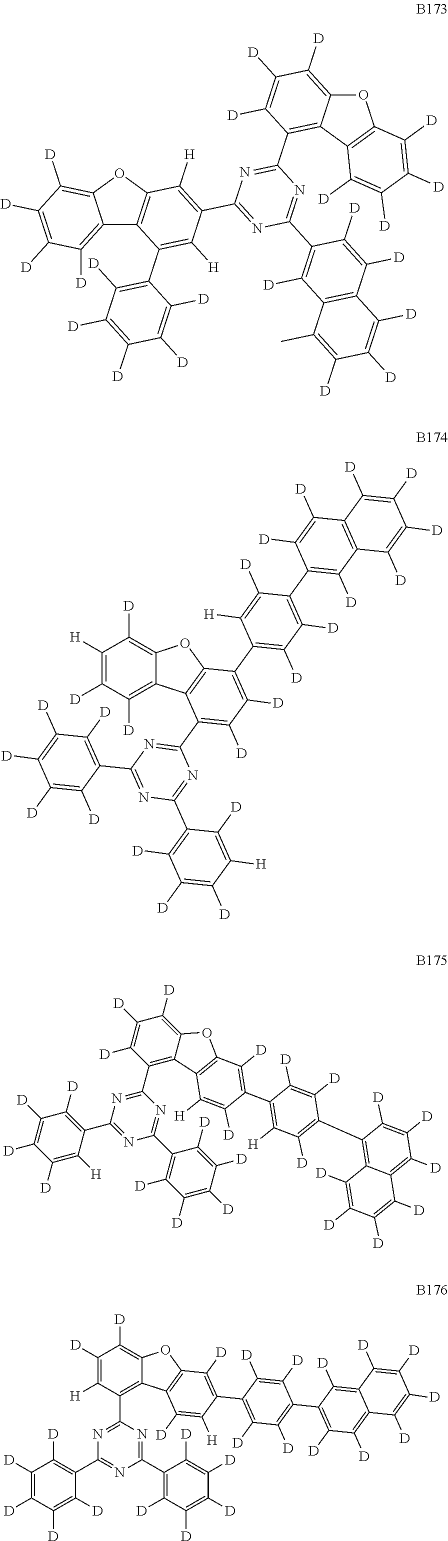

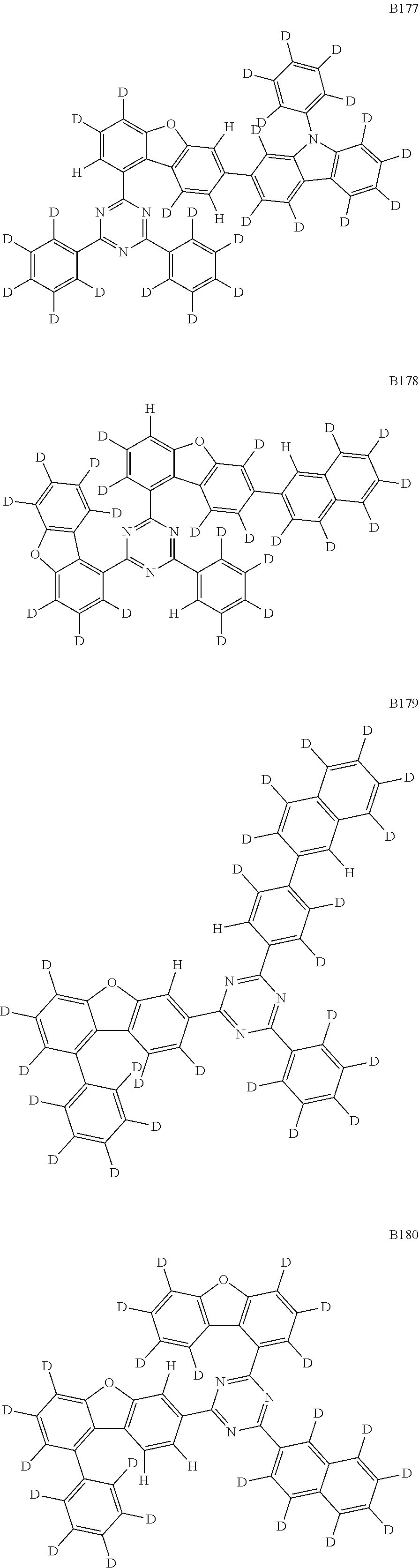

As the material of the hole injection layer 301, known hole injection layer materials may be used. For example, phthalocyanine compounds such as copper phthalocyanine disclosed in U.S. Pat. No. 4,356,429, or a starburst amine derivative described in the literature[Advanced Materials, 6, p. 677 (1994)], such as tris(4-carbazoyl-9-yiphenyl)amine (TCTA), 4,4′,4″-tri[phenyl(m-tolyl)amino]triphenylamine (m-MIDATA), 1,3,5-tris[4-(3-methylphenylphenylamino)phenyl]benzene (m-MTDAPB), 4,4′,4″-tris(N,N-(2-naphthyl)-phenylamino)triphenylamine (2-TNATA), or a soluble conductive polymer such as polyaniline/dodecylbenzenesulfonic acid, poly(3,4-ethylenedioxythiophene)/poly(4-styrenesulfonate), polyaniline/camphorsulfonic acid, or polyaniline/poly(4-styrenesulfonate) may be used.

As the materials of the hole transport layer 302, pyrazoline derivatives, arylamine derivatives, stilbene derivatives, or triphenyldiamine derivatives may be used, and low-molecular-weight or high-molecular-weight materials may also be used. As the materials of the electron transport layer, oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, or 8-hydroxyquinoline and metal complexes thereof may be used. Not only low-molecular-weight materials but also high-molecular-weight materials may be used.

For example, LiF is typically used in the art as the material of the electron injection layer 305, but the present application is not limited thereto.

As the materials of the electron transport layer 304, anthracene compounds, Alq₃(tris(8-hydroxyquinolinato)aluminum), 1,3,5-tri[(3-pyridyl)-phen-3-yl]benzene, 2,4,6-tris(3′-(pyridin-3-yl) biphenyl-3-yl)-1,3,5-triazine, 2-(4-(N-phenylbenzoimidazol-1-yl)phenyl)-9,10-dinaphthylanthracene, TPBi (1,3,5-tri (1-phenyl-1H-benzo[d]imidazol-2-yl)benzene), BCP (2,9-dimethyl-4,7-diphenyl-1,10-phenanthroline), Bphen (4,7-diphenyl-1,10-phenanthroline), TAZ (3-(4-biphenylyl)-4-phenyl-5-tert-butylphenyl-1,2,4-triazole), NTAZ (4-(naphthalen-1-yl)-3,5-diphenyl-4H-1,2,4-triazole), tBu-PBD (2-(4-biphenylyl)-5-(4-tert-butylphenyl)-1,3,4-oxadiazole), BAlq (bis(2-methyl-8-quinolinolato-N1,08)-(1,1′-biphenyl-4-olato)aluminum), Bebq2 (beryllium bis(benzoquinolin-10-olate), ADN (9,10-di(naphthalene-2-yl) anthracene), BmPyPhB (1,3-bis[3,5-di(pyridin-3-yl)phenyl]benzene), or a combination thereof may be used.

As used herein, the charge generation layer may be an N-type charge generation layer, and may further include a dopant known in the art.

According to some embodiments, a method for manufacturing the light-emitting device may be provided.

According to some embodiments, a substrate may be prepared. A first electrode may be formed on the substrate. One or more organic layers may be formed on the first electrode. A second electrode may be formed on the organic layer to manufacture a light-emitting device.

The light-emitting device may be manufactured using conventional methods and materials for manufacturing a light-emitting device, except that one or more organic layers are formed using the above-described heterocyclic compound.

When manufacturing an organic light-emitting device, an organic layer including the heterocyclic compound may be formed using a vacuum deposition method. Alternatively, the organic layer may be formed by applying a composition for an organic layer including the heterocyclic compound using a solution application method. Here, the solution application method may include spin coating, dip coating, inkjet printing, screen printing, spray coating, or roll coating, but is not limited thereto.

The organic layer may be formed using a composition for an organic layer including the heterocyclic compound represented by Chemical Formula 1.

The composition for an organic layer of an organic light-emitting device according to the present disclosure may include the heterocyclic compound represented by Chemical Formula 1 and the compound represented by Chemical Formula 4. The two compounds may function as a dual host.

The organic layer may be formed by pre-mixing the heterocyclic compound represented by Chemical Formula 1 and the compound represented by Chemical Formula 4 and depositing the mixture by a thermal vacuum deposition method.

The ratio of the weight of the compound represented by Chemical Formula 4 to the weight of the heterocyclic compound represented by Chemical Formula 1, based on the total weight of the composition for an organic layer, may be 0.1 to 10. In some embodiments, the ratio of the weight of the compound represented by Chemical Formula 4 to the weight of the heterocyclic compound represented by Chemical Formula 1, based on the total weight of the composition for an organic layer, may be 0.2 to 5, or 0.5 to 2.

The composition may be used in forming the organic layer of an organic light-emitting device, and in particular, preferably used as a host material for the emission layer.

The composition is in a form in which two or more compounds are simply mixed, and may be prepared by mixing powdered materials prior to forming the organic layer of an organic light-emitting device, or by mixing compounds that are liquid at a temperature above a predetermined temperature. The composition is in a solid state at or below the melting point of each material and may be maintained in a liquid state under high-temperature conditions.

The composition may further include materials known in the art, such as solvents and additives.

Hereinafter, embodiments of the present invention will be further described with reference to specific experimental examples. The examples and comparative examples included in the experimental examples are merely illustrative of the present disclosure and do not limit the scope of the appended claims. It will be apparent to those skilled in the art that various changes and modifications to the examples can be made within the scope and technical spirit of the present disclosure, and it is also understood that such changes and modifications fall within the scope of the appended claims.

### Preparative Example 1

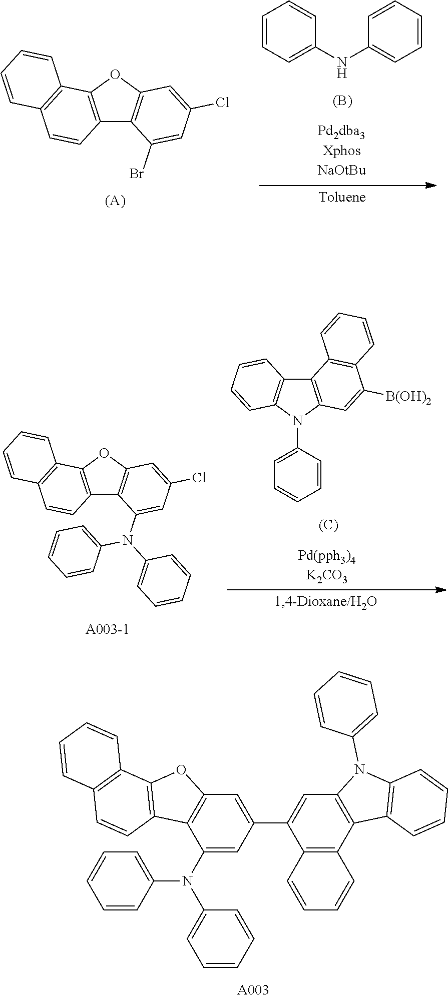

### 1) Preparation of Intermediate A003-1

10.0 g (30.31 mmol) of compound 7-bromo-9-chloronaphtho[1,2-b]benzofuran (A), 5.12 g (30.31 mmol) of diphenylamine (B), 0.34 g (1.5 mmol) of tris(dibenzylideneacetone) dipalladium (0) (Pd₂(dba)₃), 1.4 g (60.62 mmol) of 2-dicyclohexylphosphino-2′,4′,6′-triisopropylbiphenyl (XPhos), and 5.8 g (60.62 mmol) of sodium tert-butoxide (NaOtBu) were added into a 500 mL two-neck flask, dissolved in toluene (150 mL), and refluxed for 1 hour. The reaction mixture was purified by recrystallization from methanol to obtain 11.43 g (yield: 90%) of Intermediate A003-1.

### 2) Preparation of Compound A003

11.43 g (27.28 mmol) of Intermediate A003-1, 9.2 g (27.28 mmol) of (7-phenyl-7H-benzo[c]carbazol-5-yl) boronic acid (C), 1.58 g (1.36 mmol) of tetrakis(triphenylphosphine)palladium (0), (Pd(pph₃)₄), and 11.31 g (81.83 mmol) of potassium carbonate (K₂CO₃) were dissolved in a mixed solvent of 100 mL of 1,4 dioxane and 20 mL of water in a 1 L two-neck flask, and the mixture was refluxed for 1 hour. The reaction mixture was purified by recrystallization from methanol, and 16.97 g (yield 92%) of Compound A003 was obtained.

The compounds in Table 1 were synthesized in the same manner as above, except that (A) in Table 1 was used instead of 7-bromo-9-chloronaphtho[1,2-b]benzofuran (A) in step 1, (B) in Table 1 was used instead of diphenylamine (B), and (C) in Table 1 was used instead of (7-phenyl-7H-benzo[c]carbazol-5-yl) boronic acid (C) in step 2.

TABLE 1

| Compound | | | | |
|---|---|---|---|---|
| No. | (A) | (B) | (C) | Yield |
| A003 | 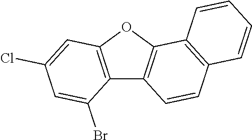 | 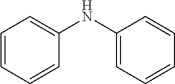 | 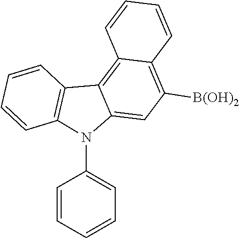 | 92% |
| A004 | 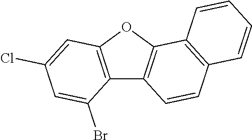 | 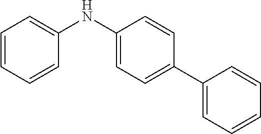 | 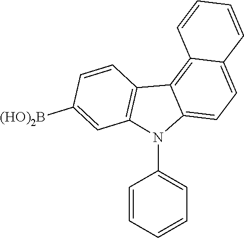 | 95% |
| A005 | 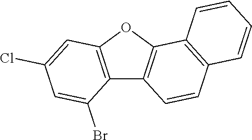 | 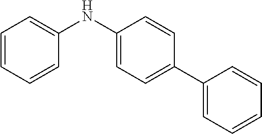 | 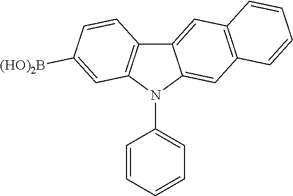 | 91% |
| A007 | 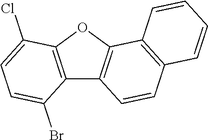 | 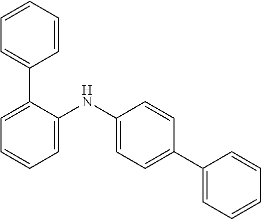 | 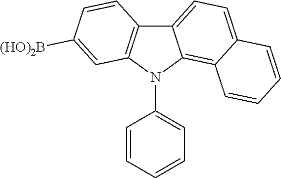 | 89% |
| A017 | 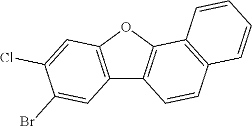 | 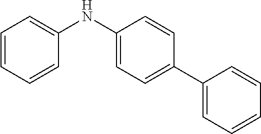 | 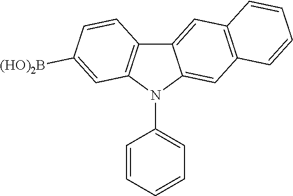 | 95% |
| A055 | 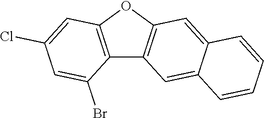 | 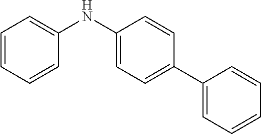 | 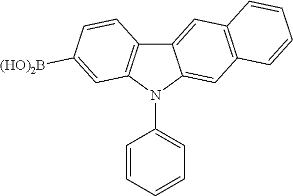 | 93% |
| A059 | 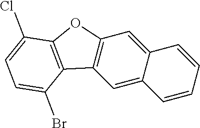 | 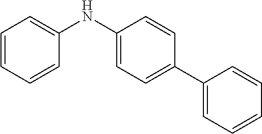 | 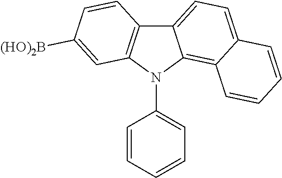 | 91% |
| A061 | 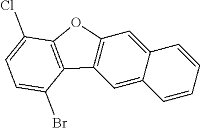 | 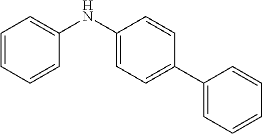 | 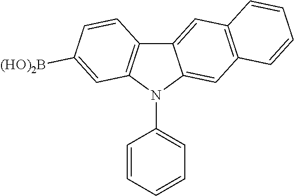 | 90% |
| A137 | 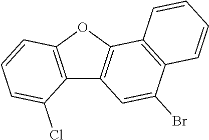 | 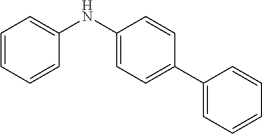 | 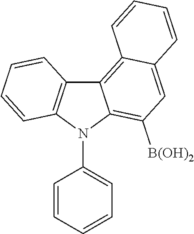 | 90% |
| A141 | 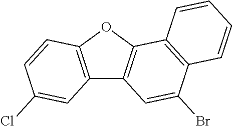 | 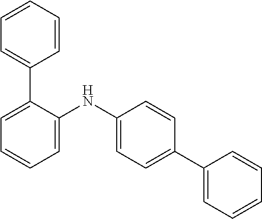 | 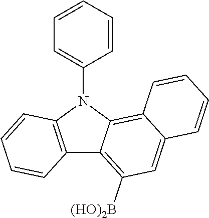 | 92% |
| A145 | 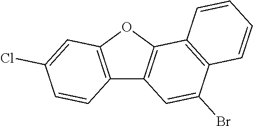 | 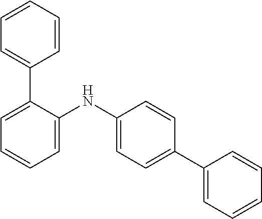 | 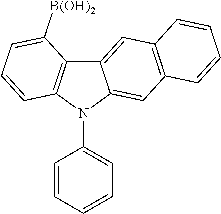 | 95% |
| A148 | 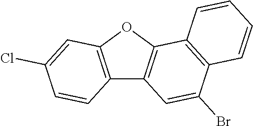 | 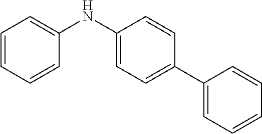 | 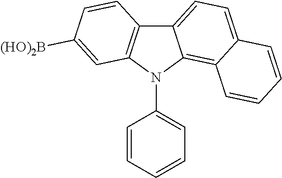 | 92% |
| A152 | 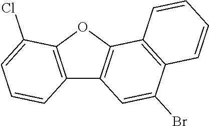 | 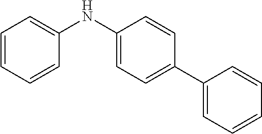 | 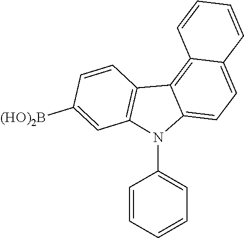 | 90% |
| A165 | 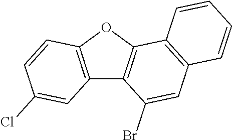 | 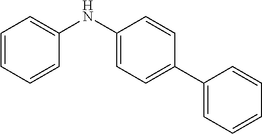 | 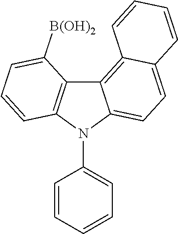 | 91% |
| A171 | 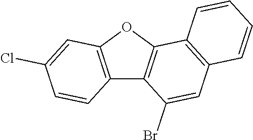 | 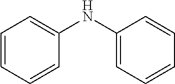 | 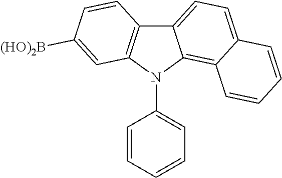 | 89% |
| A174 | 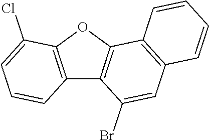 | 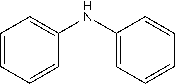 | 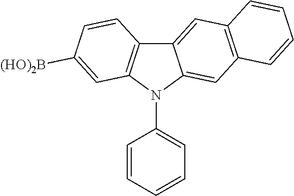 | 88% |
| A184 | 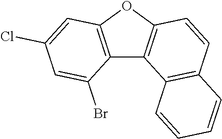 | 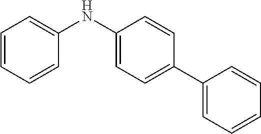 | 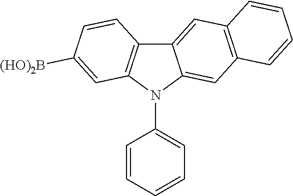 | 92% |
| A188 | 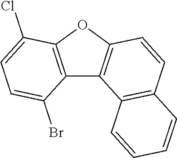 | 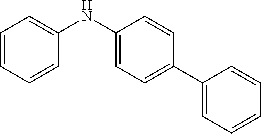 | 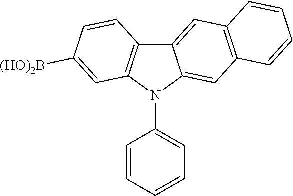 | 92% |
| A241 | 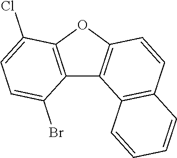 | 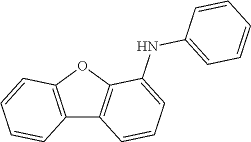 | 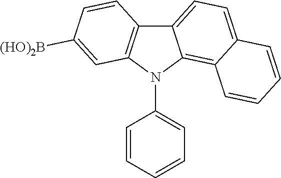 | 93% |
| A242 | 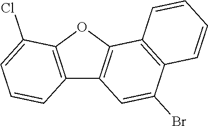 | 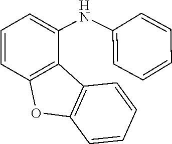 | 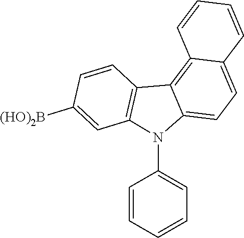 | 94% |
| A257 | 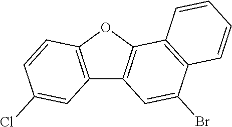 | 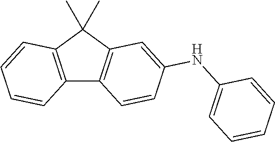 | 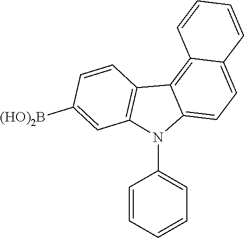 | 92% |
| A292 | 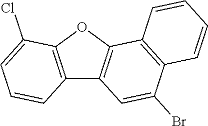 | 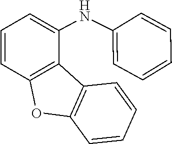 | 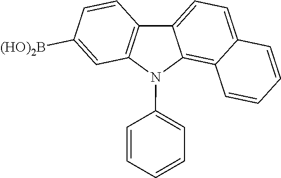 | 90% |
| A442 | 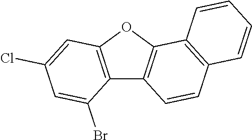 | 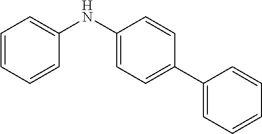 | 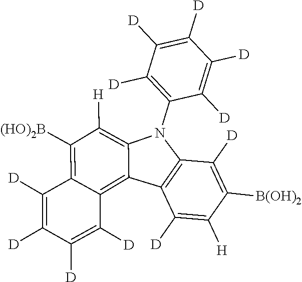 | 85% |
| A474 | 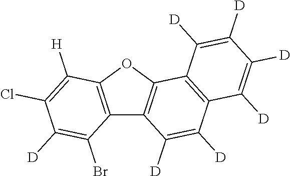 | 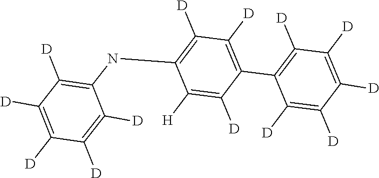 | 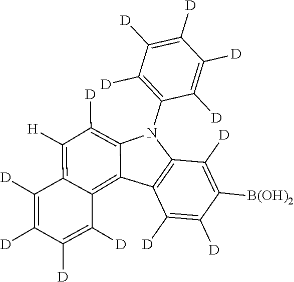 | 83% |

### Preparative Example 2

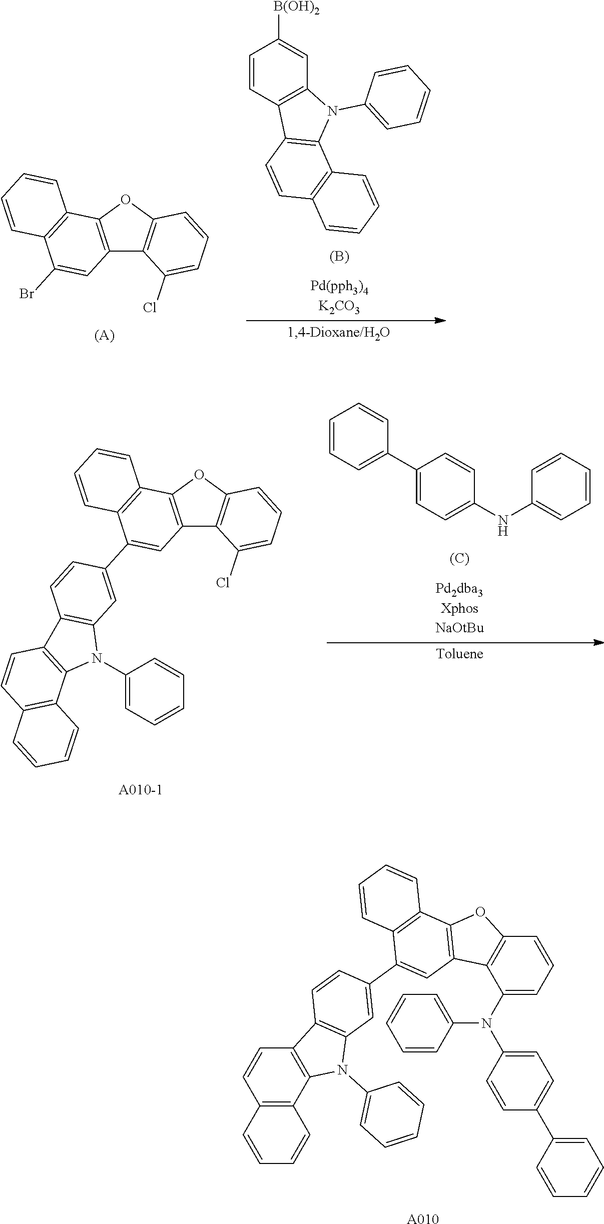

### 1) Preparation of Intermediate A010-1

10.0 g (30.31 mmol) of 5-bromo-7-chloronaphtho[1,2-b]benzofuran (A), 10.22 g (30.31 mmol) of (11-phenyl-11H-benzo[a]carbazol-9-yl) boronic acid (B), 1.75 g (0.152 mmol) of tetrakis(triphenylphosphine)palladium(0), Pd(pph₃)₄, and 12.57 g (90.93 mmol) of potassium carbonate (K₂CO₃) were dissolved in a mixed solvent of 100 mL of 1,4 dioxane and 20 mL of water in a 1 L two-neck flask, and the mixture was refluxed for 1 hour. The reaction mixture was purified by recrystallization from methanol, and 15.31 g (yield 93%) of Intermediate A010-1 was obtained.

### 2) Preparation of Compound A010

10.0 g (18.4 mmol) of Intermediate A010-1, 4.5 g (18.4 mmol) of N-phenyl-[1,1′-biphenyl]-4-amine (C), 0.84 g (0.92 mmol) of tris(dibenzylideneacetone)dipalladium(0)(Pd₂(dba)₃), 0.88 g (1.84 mmol) of 2-dicyclohexylphosphino-2′,4′,6′-triisopropylbiphenyl (XPhos), and 5.31 g (55.23 mmol) of sodium tert-butoxide (NaOtBu) were added into a 500 mL two-neck flask, dissolved in toluene (150 mL), and refluxed for 1 hour. The reaction mixture was purified by recrystallization from methanol to obtain 12.33 g (yield: 89%) of Compound A010.

Compounds in Table 2 were synthesized in the same manner as above, except that (A) in Table 2 was used instead of 5-bromo-7-chloronaphtho[1,2-b]benzofuran (A) in step 1, (B) in Table 2 was used instead of (11-phenyl-11H-benzo[a]carbazol-9-yl) boronic acid (B), and (C) in Table 2 was used instead of N-phenyl-[1,1′-biphenyl]-4-amine (C) in step 2.

TABLE 2

| Com- | | | | |
|---|---|---|---|---|
| pound | | | | |
| No. | (A) | (C) | (B) | Yield |
| A010 | 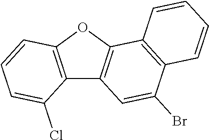 [SMILES: Clc1cccc2oc3c4ccccc4c(Br)cc3c12] | 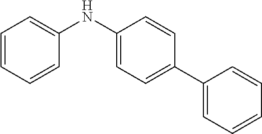 [SMILES: c1ccc(Nc2ccc(-c3ccccc3)cc2)cc1] | 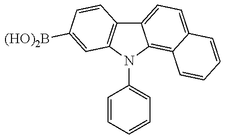 [SMILES: OB(O)c1ccc2c3ccc4ccccc4c3n(-c3ccccc3)c2c1] | 89% |
| A011 | 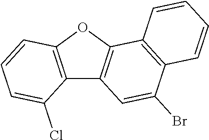 [SMILES: Clc1cccc2oc3c4ccccc4c(Br)cc3c12] | 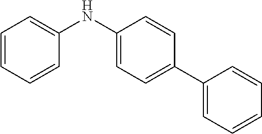 [SMILES: c1ccc(Nc2ccc(-c3ccccc3)cc2)cc1] | 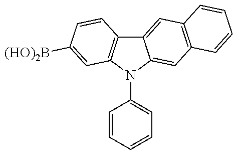 [SMILES: OB(O)c1ccc2c3cc4ccccc4cc3n(-c3ccccc3)c2c1] | 85% |
| A013 | 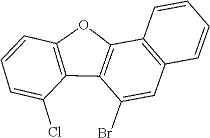 [SMILES: Clc1cccc2oc3c4ccccc4cc(Br)c3c12] | 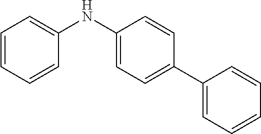 [SMILES: c1ccc(Nc2ccc(-c3ccccc3)cc2)cc1] | 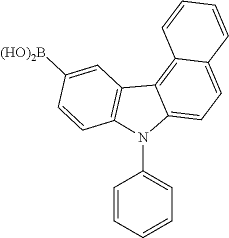 [SMILES: OB(O)c1ccc2c(c1)c1c3ccccc3ccc1n2-c1ccccc1] | 92% |
| A016 | 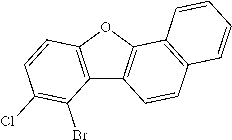 [SMILES: Clc1ccc2oc3c4ccccc4ccc3c2c1Br] | 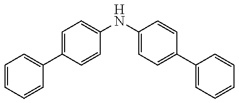 [SMILES: c1ccc(-c2ccc(Nc3ccc(-c4ccccc4)cc3)cc2)cc1] | 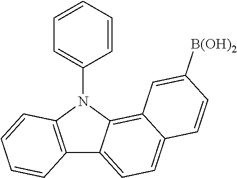 [SMILES: OB(O)c1ccc2ccc3c4ccccc4n(-c4ccccc4)c3c2c1] | 87% |
| A020 | 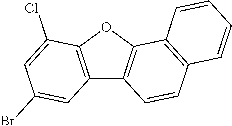 [SMILES: Clc1cc(Br)cc2c1oc1c3ccccc3ccc21] | 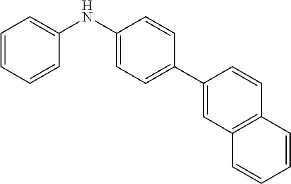 [SMILES: c1ccc(Nc2ccc(-c3ccc4ccccc4c3)cc2)cc1] | 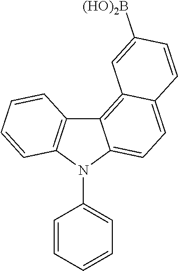 [SMILES: OB(O)c1ccc2ccc3c(c2c1)c1ccccc1n3-c1ccccc1] | 93% |
| A021 | 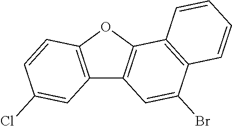 [SMILES: Clc1ccc2oc3c4ccccc4c(Br)cc3c2c1] | 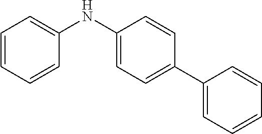 [SMILES: c1ccc(Nc2ccc(-c3ccccc3)cc2)cc1] | 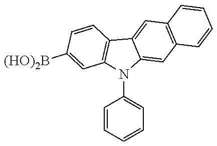 [SMILES: OB(O)c1ccc2c3cc4ccccc4cc3n(-c3ccccc3)c2c1] | 92% |
| A027 | 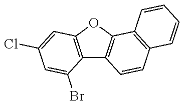 [SMILES: Clc1cc(Br)c2c(c1)oc1c3ccccc3ccc12] | 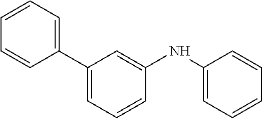 [SMILES: c1ccc(Nc2cccc(-c3ccccc3)c2)cc1] | 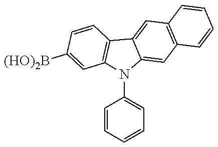 [SMILES: OB(O)c1ccc2c3cc4ccccc4cc3n(-c3ccccc3)c2c1] | 90% |
| A033 | 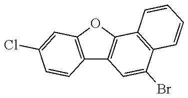 [SMILES: Clc1ccc2c(c1)oc1c3ccccc3c(Br)cc21] | 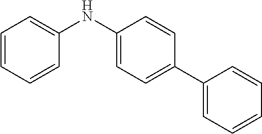 [SMILES: c1ccc(Nc2ccc(-c3ccccc3)cc2)cc1] | 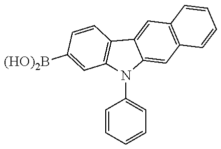 [SMILES: OB(O)c1ccc2c3cc4ccccc4cc3n(-c3ccccc3)c2c1] | 91% |
| A035 | 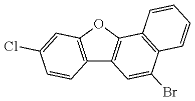 [SMILES: Clc1ccc2c(c1)oc1c3ccccc3c(Br)cc21] | 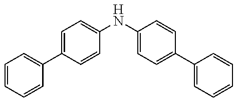 [SMILES: c1ccc(-c2ccc(Nc3ccc(-c4ccccc4)cc3)cc2)cc1] | 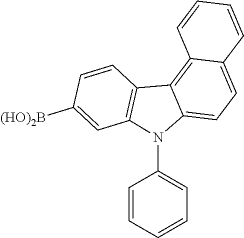 [SMILES: OB(O)c1ccc2c3c4ccccc4ccc3n(-c3ccccc3)c2c1] | 86% |
| A037 | 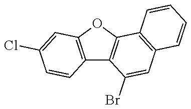 [SMILES: Clc1ccc2c(c1)oc1c3ccccc3cc(Br)c21] | 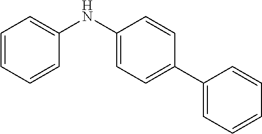 [SMILES: c1ccc(Nc2ccc(-c3ccccc3)cc2)cc1] | 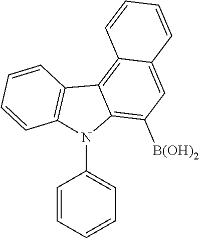 [SMILES: OB(O)c1cc2ccccc2c2c3ccccc3n(-c3ccccc3)c12] | 82% |
| A039 | 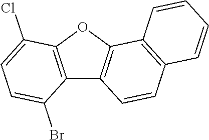 [SMILES: Clc1ccc(Br)c2c1oc1c3ccccc3ccc12] | 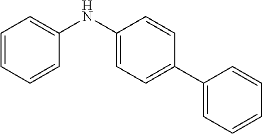 [SMILES: c1ccc(Nc2ccc(-c3ccccc3)cc2)cc1] | 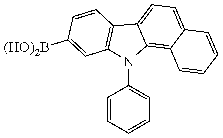 [SMILES: OB(O)c1ccc2c3ccc4ccccc4c3n(-c3ccccc3)c2c1] | 91% |
| A047 | 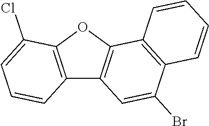 [SMILES: Clc1cccc2c1oc1c3ccccc3c(Br)cc21] | 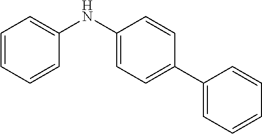 [SMILES: c1ccc(Nc2ccc(-c3ccccc3)cc2)cc1] | 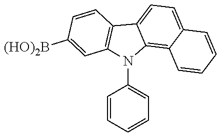 [SMILES: OB(O)c1ccc2c3ccc4ccccc4c3n(-c3ccccc3)c2c1] | 90% |
| A066 | 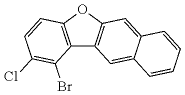 [SMILES: Clc1ccc2oc3cc4ccccc4cc3c2c1Br] | 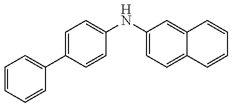 [SMILES: c1ccc(-c2ccc(Nc3ccc4ccccc4c3)cc2)cc1] | 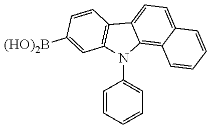 [SMILES: OB(O)c1ccc2c3ccc4ccccc4c3n(-c3ccccc3)c2c1] | 88% |
| A074 | 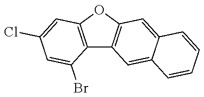 [SMILES: Clc1cc(Br)c2c(c1)oc1cc3ccccc3cc12] | 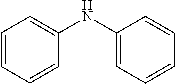 [SMILES: c1ccc(Nc2ccccc2)cc1] | 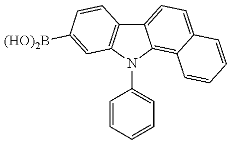 [SMILES: OB(O)c1ccc2c3ccc4ccccc4c3n(-c3ccccc3)c2c1] | 91% |
| A085 | 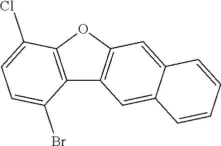 [SMILES: Clc1ccc(Br)c2c1oc1cc3ccccc3cc12] | 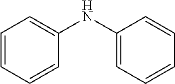 [SMILES: c1ccc(Nc2ccccc2)cc1] | 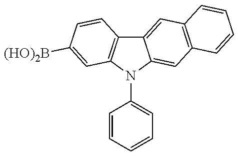 [SMILES: OB(O)c1ccc2c3cc4ccccc4cc3n(-c3ccccc3)c2c1] | 87% |
| A180 | 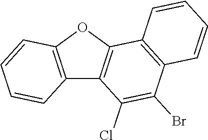 [SMILES: Clc1c(Br)c2ccccc2c2oc3ccccc3c12] | 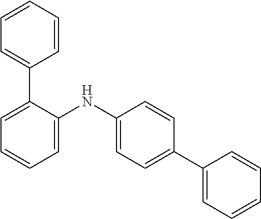 [SMILES: c1ccc(-c2ccc(Nc3ccccc3-c3ccccc3)cc2)cc1] | 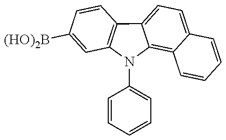 [SMILES: OB(O)c1ccc2c3ccc4ccccc4c3n(-c3ccccc3)c2c1] | 88% |
| A194 | 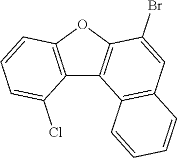 [SMILES: Clc1cccc2oc3c(Br)cc4ccccc4c3c12] | 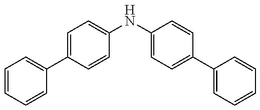 [SMILES: c1ccc(-c2ccc(Nc3ccc(-c4ccccc4)cc3)cc2)cc1] | 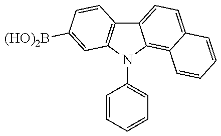 [SMILES: OB(O)c1ccc2c3ccc4ccccc4c3n(-c3ccccc3)c2c1] | 86% |
| A195 | 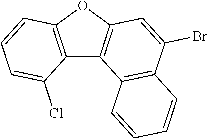 [SMILES: Clc1cccc2oc3cc(Br)c4ccccc4c3c12] | 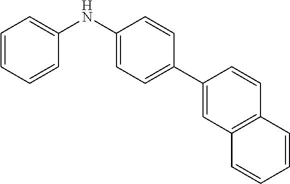 [SMILES: c1ccc(Nc2ccc(-c3ccc4ccccc4c3)cc2)cc1] | 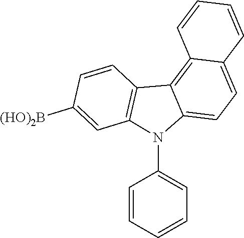 [SMILES: OB(O)c1ccc2c3c4ccccc4ccc3n(-c3ccccc3)c2c1] | 92% |
| A220 | 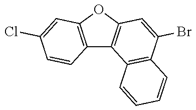 [SMILES: Clc1ccc2c(c1)oc1cc(Br)c3ccccc3c12] | 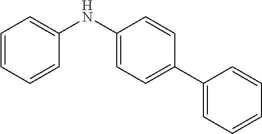 [SMILES: c1ccc(Nc2ccc(-c3ccccc3)cc2)cc1] | 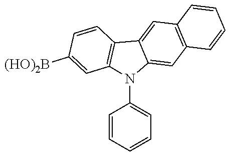 [SMILES: OB(O)c1ccc2c3cc4ccccc4cc3n(-c3ccccc3)c2c1] | 95% |
| A223 | 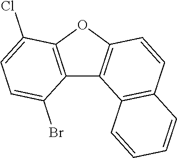 [SMILES: Clc1ccc(Br)c2c1oc1ccc3ccccc3c12] | 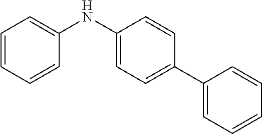 [SMILES: c1ccc(Nc2ccc(-c3ccccc3)cc2)cc1] | 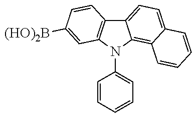 [SMILES: OB(O)c1ccc2c3ccc4ccccc4c3n(-c3ccccc3)c2c1] | 87% |
| A265 | 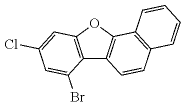 [SMILES: Clc1cc(Br)c2c(c1)oc1c3ccccc3ccc12] | 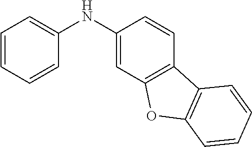 [SMILES: c1ccc(Nc2ccc3c(c2)oc2ccccc23)cc1] | 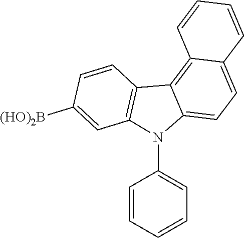 [SMILES: OB(O)c1ccc2c3c4ccccc4ccc3n(-c3ccccc3)c2c1] | 90% |
| A448 | 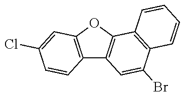 [SMILES: Clc1ccc2c(c1)oc1c3ccccc3c(Br)cc21] | 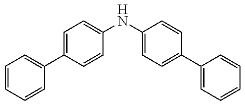 [SMILES: c1ccc(-c2ccc(Nc3ccc(-c4ccccc4)cc3)cc2)cc1] | 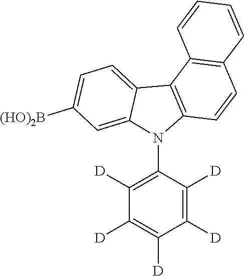 [SMILES: [2H]c1c([2H])c([2H])c(-n2c3cc(B(O)O)ccc3c3c4ccccc4ccc32)c([2H])c1[2H]] | 84% |

### Preparative Example 3

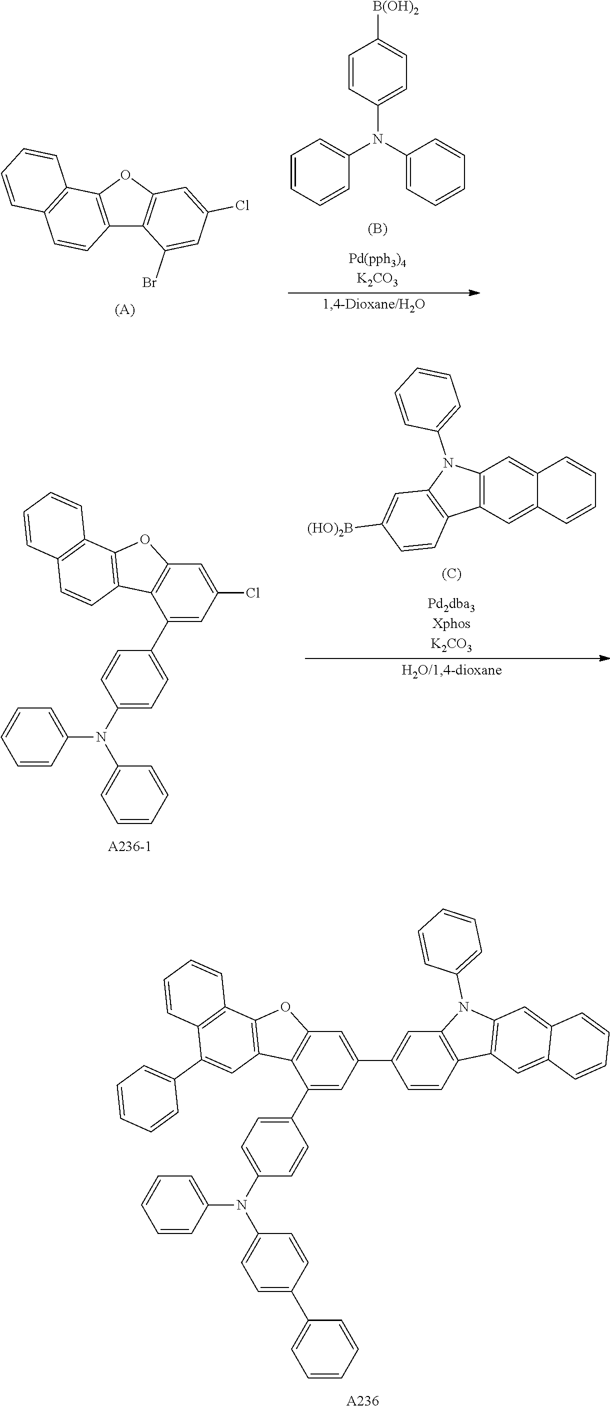

### 1) Preparation of Intermediate A236-1

10.0 g (30.31 mmol) of 7-bromo-9-chloronaphtho[1,2-b]benzofuran (A), 8.76 g (30.31 mmol) of (4-(diphenylamino)phenyl) boronic acid (B), 1.75 g (1.52 mmol) of tetrakis(triphenylphosphine)palladium (0), (Pd(pph₃))₄), and 12.57 g (90.93 mmol) of K₂CO₃ were dissolved in a mixed solvent of 100 mL of 1,4-dioxane and 20 mL of water in a 1 L two-neck flask, and the mixture was refluxed for 1 hour. The reaction mixture was purified by recrystallization from methanol, and 13.36 g (yield 89%) of Intermediate A236-1 was obtained.

### 2) Preparation of Compound A236

10.0 g (20.20 mmol) of Intermediate A236-1, 6.81 g (20.20 mmol) of (5-phenyl-5H-benzo[b]carbazol-3-yl) boronic acid (C), 0.92 g (1.01 mmol) of tris(dibenzylideneacetone) dipalladium (0), (Pd₂(dba)₃), 0.96 g (2.02 mmol) of 2-dicyclohexylphosphino-2′,4′,6′-triisopropylbiphenyl (XPhos), and 8.37 g (60.59 mmol) of K₂CO₃ were dissolved in a mixed solvent of 100 mL of 1,4-dioxane and 20 mL of water in a 250 mL two-neck flask, and the mixture was refluxed for 1 hour. The reaction mixture was purified by recrystallization from methanol to obtain 16.26 g (89% yield) of Compound A236.

Compounds in Table 3 were synthesized in the same manner as above, except that (A) in Table 3 was used instead of 7-bromo-9-chloronaphtho[1,2-b]benzofuran (A) in step 1, (B) in Table 3 was used instead of (4-(diphenylamino)phenyl) boronic acid (B), and (C) in Table 3 was used instead of (5-phenyl-5H-benzo[b]carbazol-3-yl) boronic acid (C) in step 2.

TABLE 3

| Compound | | |
|---|---|---|
| No. | (A) | (B) |
| A236 | 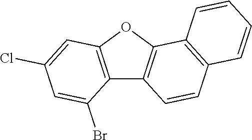 | 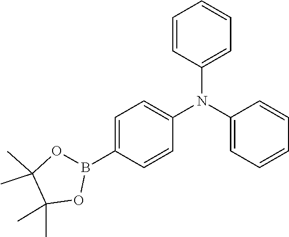 |
| A240 | 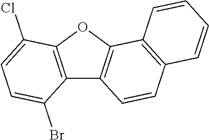 | 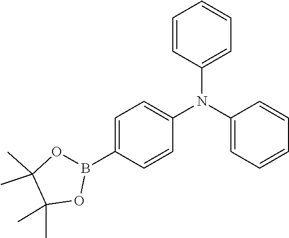 |
| A256 | 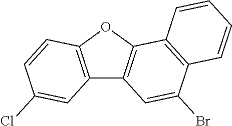 | 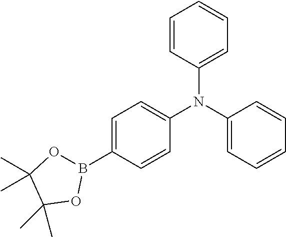 |
| A264 | 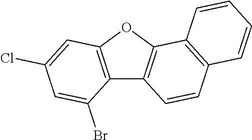 | 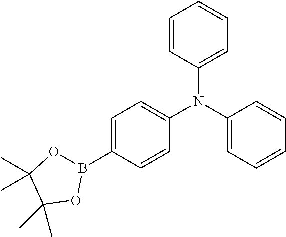 |
| A269 | 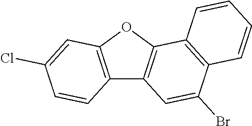 | 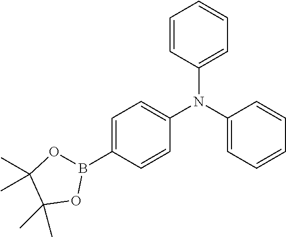 |
| A275 | 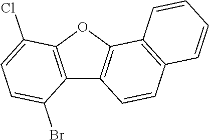 | 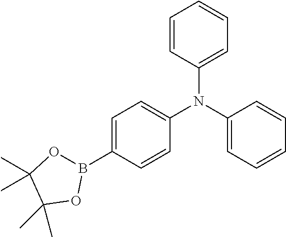 |
| A281 | 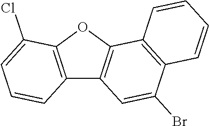 | 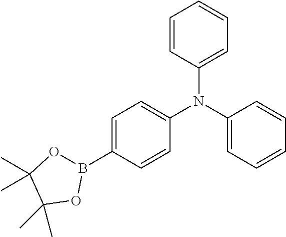 |
| A324 | 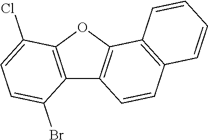 | 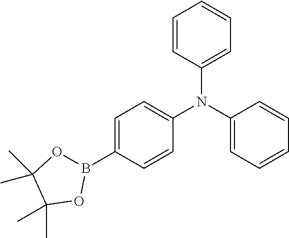 |
| A352 | 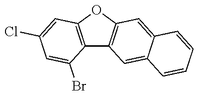 | 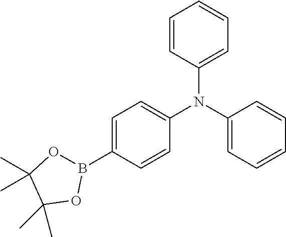 |
| A428 | 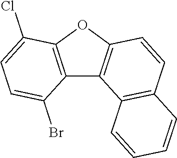 | 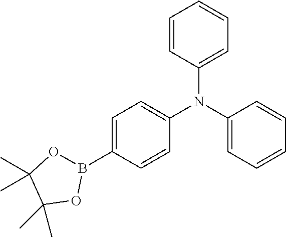 |
| A432 | 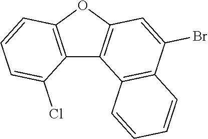 | 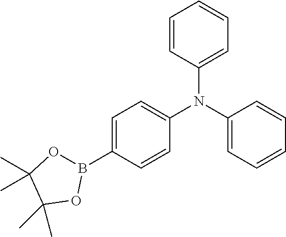 |
| A480 | 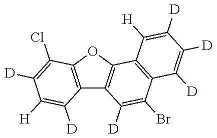 | 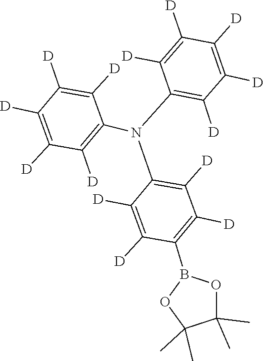 |

| Compound | | |
|---|---|---|
| No. | (C) | Yield |
| A236 | 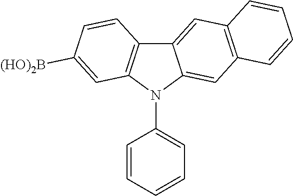 | 89% |
| A240 | 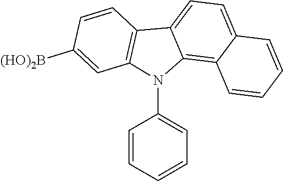 | 88% |
| A256 | 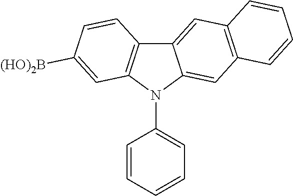 | 86% |
| A264 | 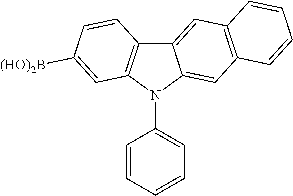 | 92% |
| A269 | 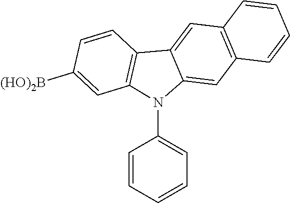 | 95% |
| A275 | 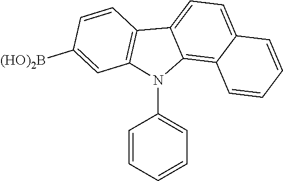 | 87% |
| A281 | 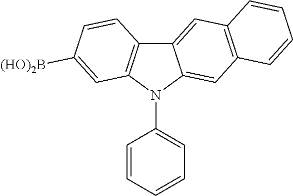 | 92% |
| A324 | 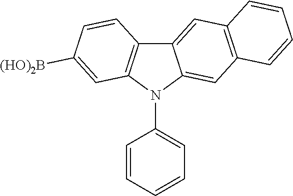 | 90% |
| A352 | 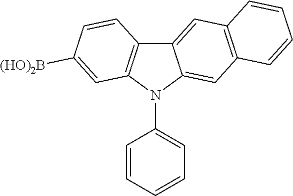 | 91% |
| A428 | 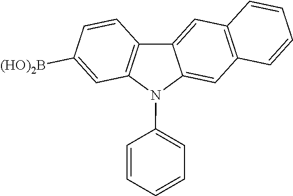 | 86% |
| A432 | 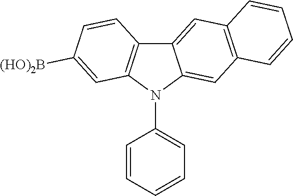 | 82% |
| A480 | 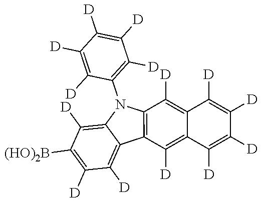 | 84% |

The synthesis results for the compounds in Tables 1 to 3 are shown in Tables 4 and 5 below. Table 4 shows the ¹H NMR (400 MHZ. CDCl₃) measurements, and Table 5 shows the field-desorption mass spectrometry (FD-MS) measurements.

TABLE 4

| Compound | |
|---|---|
| No. | ¹H NMR (400 MHz, CDCl₃) |
| A003 | 8.55 (2H, s), 8.16 (2H, d), 8.12 (1H, d), 7.89 (1H, s), 7.67 (2H, d), 7.63 (1H, s), 7.62 (1H, |
| | d), 7.58 (2H, d), 7.55 (2H, d), 7.53 (1H, dd), 7.51 (1H, d), 7.5 (3H, d), 7.45 (1H, dd), 7.29 |
| | (1H, dd), 7.2 (4H, s), 7 (1H, t), 6.81 (2H, t), 6.63 (4H, dd) |
| A004 | 8.54 (1H, s), 8.18 (1H, d), 8.16 (3H, d), 7.96 (1H, s), 7.94 (1H, d), 7.89 (1H, s), 7.79 (1H, |
| | d), 7.67 (4H, d), 7.62 (1H, d), 7.58 (2H, dd), 7.54 (2H, d), 7.53 (1H, d), 7.52 (2H, dd), 7.51 |
| | (3H, dd), 7.5 (2H, s), 7.45 (1H, t), 7.41 (1H, t), 7.2 (2H, dd), 7 (1H, dd), 6.81 (1H, t), 6.69 |
| | (2H, d), 6.63 (2H, d) |
| A005 | 8.18 (1H, s), 8.16 (4H, d), 7.89 (1H, d), 7.79 (1H, s), 7.67 (4H, d), 7.62 (1H, s), 7.58 (2H, |
| | d), 7.55 (1H, d), 7.54 (2H, d), 7.53 (1H, dd), 7.52 (2H, d), 7.51 (3H, d), 7.5 (2H, dd), 7.45 |
| | (1H, dd), 7.41 (1H, s), 7.4 (1H, t), 7.2 (2H, t), 7 (1H, dd), 6.81 (1H, dd), 6.69 (2H, t), 6.63 |
| | (2H, d) |
| A007 | 8.51 (1H, s), 8.43 (1H, d), 8.18 (1H, d), 8.16 (3H, s), 7.89 (1H, d), 7.88 (1H, s), 7.79 (1H, |
| | d), 7.67 (4H, d), 7.62 (1H, d), 7.58 (2H, dd), 7.56 (1H, d), 7.54 (3H, d), 7.53 (1H, dd), 7.52 |
| | (2H, dd), 7.51 (4H, s), 7.5 (2H, t), 7.16 (1H, t), 7.08 (2H, dd), 6.87 (1H, dd), 6.69 (3H, t), |
| | 6.39 (1H, d) |
| A010 | 8.55 (1H, s), 8.51 (1H, d), 8.49 (1H, d), 8.18 (1H, s), 8.16 (1H, d), 8.12 (1H, s), 8.1 (1H, d), |
| | 7.71 (1H, d), 7.67 (2H, d), 7.62 (1H, dd), 7.58 (2H, d), 7.57 (1H, d), 7.55 (2H, dd), 7.54 |
| | (2H, dd), 7.52 (2H, s), 7.51 (2H, t), 7.5 (2H, t), 7.45 (1H, dd), 7.41 (1H, dd), 7.2 (2H, t), |
| | 7.13 (1H, d), 7.02 (1H, d), 6.81 (1H, d), 6.69 (2H, dd), 6.63 (2H, t), 6.33 (1H, d) |
| A011 | 8.55 (1H, s), 8.49 (1H, d), 8.18 (1H, d), 8.16 (2H, s), 8.1 (1H, d), 7.71 (1H, s), 7.67 (2H, d), |
| | 7.62 (1H, d), 7.58 (2H, d), 7.55 (3H, dd), 7.54 (2H, d), 7.52 (2H, d), 7.51 (2H, dd), 7.5 (2H, |
| | dd), 7.45 (1H, s), 7.41 (1H, t), 7.4 (1H, t), 7.2 (2H, dd), 7.13 (1H, dd), 7.02 (1H, t), 6.81 |
| | (1H, d), 6.69 (2H, d), 6.63 (2H, d), 6.33 (1H, dd) |
| A013 | 8.54 (1H, s), 8.18 (1H, d), 8.16 (1H, d), 8 (3H, s), 7.77 (1H, d), 7.67 (2H, s), 7.65 (1H, d), |
| | 7.63 (1H, d), 7.59 (2H, d), 7.58 (2H, dd), 7.54 (2H, d), 7.52 (2H, d), 7.51 (2H, dd), 7.5 (2H, |
| | dd), 7.45 (1H, s), 7.41 (1H, t), 7.35 (1H, t), 7.2 (2H, dd), 7.13 (1H, dd), 7.02 (1H, t), 6.81 |
| | (1H, d), 6.69 (2H, d), 6.63 (2H, d), 6.33 (1H, dd) |
| A016 | 8.55 (1H, s), 8.34 (1H, d), 8.16 (2H, d), 8.12 (1H, s), 7.94 (1H, d), 7.92 (1H, s), 7.89 (1H, |
| | d), 7.73 (1H, d), 7.67 (2H, d), 7.58 (2H, dd), 7.57 (1H, d), 7.54 (4H, d), 7.53 (1H, dd), 7.52 |
| | (4H, dd), 7.51 (4H, s), 7.5 (2H, t), 7.37 (1H, t), 7.33 (1H, dd), 7.25 (1H, dd), 6.69 (4H, t), |
| | 6.45 (1H, d) |
| A017 | 8.18 (1H, s), 8.16 (4H, d), 7.89 (1H, d), 7.79 (1H, s), 7.71 (1H, d), 7.67 (4H, s), 7.62 (1H, |
| | d), 7.58 (2H, d), 7.55 (1H, d), 7.54 (2H, dd), 7.53 (1H, d), 7.52 (2H, d), 7.51 (2H, dd), 7.5 |
| | (2H, dd), 7.45 (1H, s), 7.41 (1H, t), 7.4 (1H, t), 7.39 (1H, dd), 7.2 (2H, dd), 6.81 (1H, t), |
| | 6.69 (2H, d), 6.63 (2H, d) |
| A020 | 8.55 (1H, s), 8.34 (1H, d), 8.16 (2H, d), 8 (2H, s), 7.94 (1H, d), 7.92 (2H, s), 7.89 (1H, d), |
| | 7.73 (2H, d), 7.67 (2H, d), 7.65 (1H, dd), 7.63 (1H, d), 7.61 (1H, d), 7.59 (2H, dd), 7.58 |
| | (3H, dd), 7.57 (1H, s), 7.54 (2H, t), 7.53 (1H, t), 7.5 (2H, dd), 7.33 (1H, dd), 7.25 (1H, t), |
| | 7.2 (2H, d), 6.69 (2H, d), 6.63 (2H, d) |
| A021 | 8.55 (1H, s), 8.18 (2H, d), 8.16 (2H, d), 7.79 (1H, s), 7.71 (1H, d), 7.67 (2H, s), 7.65 (1H, |
| | d), 7.62 (1H, d), 7.58 (2H, d), 7.55 (3H, dd), 7.54 (2H, d), 7.52 (2H, d), 7.51 (2H, dd), 7.5 |
| | (2H, dd), 7.45 (1H, s), 7.41 (2H, t), 7.4 (1H, t), 7.2 (2H, dd), 6.81 (1H, dd), 6.69 (2H, t), |
| | 6.63 (2H, d), 6.39 (1H, d) |
| A027 | 8.49 (1H, s), 8.16 (4H, d), 8.1 (1H, d), 7.89 (1H, s), 7.67 (4H, d), 7.62 (1H, s), 7.58 (2H, d), |
| | 7.55 (1H, d), 7.53 (1H, d), 7.52 (2H, dd), 7.51 (3H, d), 7.5 (2H, d), 7.45 (1H, dd), 7.44 (1H, |
| | dd), 7.41 (1H, s), 7.4 (1H, t), 7.39 (1H, t), 7.2 (2H, dd), 6.89 (1H, dd), 6.88 (1H, t), 6.81 |
| | (1H, d), 6.63 (2H, d), 6.59 (1H, d) |
| A033 | 8.55 (1H, s), 8.49 (1H, d), 8.18 (1H, d), 8.16 (2H, s), 8.1 (1H, d), 7.71 (1H, s), 7.67 (2H, d), |
| | 7.64 (1H, d), 7.62 (1H, d), 7.58 (2H, dd), 7.55 (3H, d), 7.54 (2H, d), 7.52 (2H, dd), 7.51 |
| | (2H, dd), 7.5 (2H, s), 7.45 (1H, t), 7.43 (1H, t), 7.41 (1H, dd), 7.4 (1H, dd), 7.2 (2H, t), 6.81 |
| | (1H, d), 6.69 (2H, d), 6.63 (2H, d), 6.33 (1H, dd) |
| A035 | 8.55 (1H, s), 8.54 (1H, d), 8.49 (1H, d), 8.18 (1H, s), 8.16 (1H, d), 8.1 (1H, s), 7.71 (1H, d), |
| | 7.67 (2H, d), 7.65 (1H, d), 7.64 (1H, dd), 7.63 (1H, d), 7.62 (1H, d), 7.58 (2H, dd), 7.55 |
| | (2H, dd), 7.54 (4H, s), 7.52 (4H, t), 7.51 (4H, t), 7.5 (2H, dd), 7.43 (1H, dd), 6.69 (4H, t), |
| | 6.33 (1H, d) |
| A037 | 8.18 (1H, s), 8.16 (2H, d), 8 (3H, d), 7.77 (1H, s), 7.67 (2H, d), 7.64 (1H, s), 7.59 (2H, d), |
| | 7.58 (2H, d), 7.55 (1H, d), 7.54 (2H, dd), 7.52 (2H, d), 7.51 (2H, d), 7.5 (2H, dd), 7.45 (1H, |
| | dd), 7.43 (1H, s), 7.41 (1H, t), 7.4 (1H, t), 7.35 (1H, dd), 7.2 (2H, dd), 6.81 (1H, t), 6.69 |
| | (2H, d), 6.63 (2H, d), 6.33 (1H, d) |
| A039 | 8.51 (1H, s), 8.49 (1H, d), 8.16 (3H, d), 8.12 (1H, s), 8.1 (1H, d), 7.89 (1H, s), 7.67 (4H, d), |
| | 7.62 (1H, d), 7.58 (2H, d), 7.57 (1H, dd), 7.54 (2H, d), 7.53 (1H, d), 7.52 (2H, dd), 7.51 |
| | (2H, dd), 7.5 (3H, s), 7.45 (1H, t), 7.41 (1H, t), 7.2 (2H, dd), 6.81 (1H, dd), 6.69 (2H, t), |
| | 6.63 (2H, d), 6.45 (1H, d) |
| A047 | 8.55 (1H, s), 8.51 (1H, d), 8.43 (1H, d), 8.18 (2H, s), 8.16 (1H, d), 7.88 (1H, s), 7.79 (1H, |
| | d), 7.71 (1H, d), 7.67 (2H, d), 7.62 (1H, dd), 7.58 (2H, d), 7.55 (2H, d), 7.54 (2H, dd), 7.52 |
| | (2H, dd), 7.51 (2H, s), 7.5 (2H, t), 7.45 (1H, t), 7.41 (1H, dd), 7.25 (1H, dd), 7.2 (2H, t), |
| | 7.07 (1H, d), 6.81 (1H, d), 6.69 (2H, d), 6.63 (2H, dd), 6.39 (1H, t) |
| A055 | 8.18 (1H, s), 8.16 (4H, d), 7.79 (1H, d), 7.67 (4H, s), 7.62 (1H, d), 7.58 (2H, s), 7.55 (1H, |
| | d), 7.54 (2H, d), 7.52 (2H, d), 7.51 (3H, dd), 7.5 (2H, d), 7.49 (1H, d), 7.45 (1H, dd), 7.42 |
| | (1H, dd), 7.41 (1H, s), 7.4 (1H, t), 7.2 (2H, t), 7 (1H, dd), 6.81 (1H, dd), 6.69 (2H, t), 6.63 |
| | (2H, d) |
| A059 | 8.51 (1H, s), 8.43 (1H, d), 8.18 (1H, d), 8.16 (3H, s), 7.88 (1H, d), 7.79 (1H, s), 7.67 (4H, |
| | d), 7.62 (1H, d), 7.58 (2H, d), 7.56 (1H, dd), 7.54 (2H, d), 7.52 (2H, d), 7.51 (2H, dd), 7.5 |
| | (2H, dd), 7.49 (1H, s), 7.45 (1H, t), 7.42 (1H, t), 7.41 (1H, dd), 7.2 (2H, dd), 6.81 (1H, t), |
| | 6.69 (2H, d), 6.63 (2H, d), 6.39 (1H, d) |
| A061 | 8.18 (1H, s), 8.16 (4H, d), 7.79 (1H, d), 7.67 (4H, s), 7.62 (1H, d), 7.58 (2H, s), 7.56 (1H, |
| | d), 7.55 (1H, d), 7.54 (2H, d), 7.52 (2H, dd), 7.51 (2H, d), 7.5 (2H, d), 7.49 (1H, dd), 7.45 |
| | (1H, dd), 7.42 (1H, s), 7.41 (1H, t), 7.4 (1H, t), 7.2 (2H, dd), 6.81 (1H, dd), 6.69 (2H, t), |
| | 6.63 (2H, d), 6.39 (1H, d) |
| A066 | 8.51 (1H, s), 8.49 (1H, d), 8.16 (3H, d), 8.12 (1H, s), 8.1 (1H, d), 7.88 (1H, s), 7.84 (1H, d), |
| | 7.77 (1H, d), 7.74 (1H, d), 7.67 (4H, dd), 7.62 (1H, d), 7.58 (2H, d), 7.57 (1H, dd), 7.54 |
| | (2H, dd), 7.52 (2H, s), 7.51 (2H, t), 7.5 (3H, t), 7.49 (2H, dd), 7.42 (1H, dd), 7.37 (1H, t), |
| | 7.36 (1H, d), 6.69 (2H, d), 6.45 (1H, d) |
| A074 | 8.51 (1H, s), 8.49 (1H, d), 8.16 (3H, d), 8.12 (1H, s), 8.1 (1H, d), 7.67 (4H, s), 7.62 (1H, d), |
| | 7.58 (2H, d), 7.57 (1H, d), 7.51 (1H, dd), 7.5 (2H, d), 7.49 (1H, d), 7.45 (1H, dd), 7.42 (1H, |
| | dd), 7.39 (1H, s), 7.2 (4H, t), 6.81 (2H, t), 6.63 (4H, dd) |
| A085 | 8.18 (1H, s), 8.16 (4H, d), 7.79 (1H, d), 7.67 (4H, s), 7.62 (1H, d), 7.58 (2H, s), 7.55 (1H, |
| | d), 7.5 (3H, d), 7.49 (1H, d), 7.45 (1H, dd), 7.42 (1H, d), 7.4 (1H, d), 7.2 (4H, dd), 6.81 |
| | (2H, dd), 6.63 (4H, s), 6.45 (1H, t) |
| A137 | 8.55 (1H, s), 8.18 (1H, d), 8.08 (1H, d), 8 (2H, s), 7.94 (1H, d), 7.75 (1H, s), 7.68 (1H, d), |
| | 7.62 (1H, d), 7.59 (2H, d), 7.58 (2H, dd), 7.55 (2H, d), 7.54 (2H, d), 7.52 (2H, dd), 7.51 |
| | (2H, dd), 7.5 (2H, s), 7.45 (1H, t), 7.44 (1H, t), 7.41 (1H, dd), 7.33 (1H, dd), 7.3 (1H, t), |
| | 7.25 (1H, d), 7.2 (2H, d), 6.81 (1H, d), 6.69 (2H, dd), 6.63 (2H, t) |
| A141 | 8.18 (1H, s), 8.12 (1H, d), 8.08 (1H, d), 8 (2H, s), 7.81 (1H, d), 7.72 (1H, s), 7.71 (1H, d), |
| | 7.68 (1H, d), 7.63 (1H, d), 7.59 (2H, dd), 7.58 (2H, d), 7.55 (2H, d), 7.54 (3H, dd), 7.52 |
| | (2H, dd), 7.51 (4H, s), 7.5 (3H, t), 7.29 (1H, t), 7.22 (1H, dd), 7.16 (1H, dd), 7.08 (2H, t), |
| | 6.87 (1H, d), 6.69 (3H, d) |
| A145 | 8.18 (1H, s), 8.16 (2H, d), 8.1 (1H, d), 8.08 (1H, s), 7.95 (1H, d), 7.9 (1H, s), 7.75 (1H, d), |
| | 7.68 (1H, d), 7.67 (2H, d), 7.64 (1H, dd), 7.58 (2H, d), 7.55 (3H, d), 7.54 (3H, dd), 7.52 |
| | (2H, dd), 7.51 (4H, s), 7.5 (2H, t), 7.4 (1H, t), 7.39 (1H, dd), 7.16 (1H, dd), 7.08 (2H, t), |
| | 6.87 (1H, d), 6.69 (3H, d) |
| A148 | 8.51 (1H, s), 8.43 (1H, d), 8.18 (2H, d), 8.16 (1H, s), 8.08 (1H, d), 7.95 (1H, s), 7.88 (1H, |
| | d), 7.79 (1H, d), 7.75 (1H, d), 7.68 (1H, dd), 7.67 (2H, d), 7.64 (1H, d), 7.62 (1H, dd), 7.58 |
| | (2H, dd), 7.55 (2H, s), 7.54 (2H, t), 7.52 (2H, t), 7.51 (2H, dd), 7.5 (2H, dd), 7.45 (1H, t), |
| | 7.41 (1H, d), 7.2 (2H, d), 6.81 (1H, d), 6.69 (2H, dd), 6.63 (2H, t) |
| A152 | 8.54 (1H, s), 8.18 (2H, d), 8.16 (1H, d), 8.08 (1H, s), 7.96 (1H, d), 7.94 (1H, s), 7.85 (1H, |
| | d), 7.81 (1H, d), 7.79 (1H, d), 7.68 (1H, dd), 7.67 (2H, d), 7.62 (1H, d), 7.58 (2H, dd), 7.55 |
| | (2H, dd), 7.54 (2H, s), 7.52 (2H, t), 7.51 (2H, t), 7.5 (2H, dd), 7.45 (1H, dd), 7.41 (1H, t), |
| | 7.38 (1H, d), 7.2 (2H, d), 6.81 (1H, d), 6.69 (2H, dd), 6.63 (2H, t) |
| A165 | 8.54 (1H, s), 8.16 (1H, d), 8 (2H, d), 7.96 (1H, s), 7.94 (1H, d), 7.81 (1H, s), 7.79 (1H, d), |
| | 7.72 (1H, d), 7.71 (1H, d), 7.67 (2H, dd), 7.59 (3H, d), 7.58 (2H, d), 7.54 (2H, dd), 7.52 |
| | (2H, dd), 7.51 (2H, s), 7.5 (2H, t), 7.45 (1H, t), 7.43 (1H, dd), 7.41 (1H, dd), 7.21 (1H, t), |
| | 7.2 (2H, d), 6.81 (1H, d), 6.69 (2H, d), 6.63 (2H, dd) |
| A171 | 8.51 (1H, s), 8.43 (1H, d), 8.18 (1H, d), 8.16 (1H, s), 8 (2H, d), 7.95 (1H, s), 7.88 (1H, d), |
| | 7.79 (1H, d), 7.75 (1H, d), 7.67 (2H, dd), 7.64 (1H, d), 7.62 (1H, d), 7.59 (2H, dd), 7.58 |
| | (2H, dd), 7.5 (2H, s), 7.45 (1H, t), 7.21 (1H, t), 7.2 (4H, dd), 6.81 (2H, dd), 6.63 (4H, t) |
| A174 | 8.18 (1H, s), 8.16 (2H, d), 8 (2H, d), 7.85 (1H, s), 7.81 (1H, d), 7.79 (1H, s), 7.67 (2H, d), |
| | 7.62 (1H, d), 7.59 (2H, d), 7.58 (2H, dd), 7.55 (1H, d), 7.5 (2H, d), 7.45 (1H, dd), 7.4 (1H, |
| | dd), 7.38 (1H, s), 7.21 (1H, t), 7.2 (4H, t), 6.81 (2H, dd), 6.63 (4H, dd) |
| A180 | 8.55 (1H, s), 8.51 (1H, d), 8.43 (1H, d), 8.18 (2H, s), 8.16 (1H, d), 7.89 (1H, s), 7.88 (1H, |
| | d), 7.79 (1H, d), 7.67 (2H, d), 7.66 (1H, dd), 7.62 (1H, d), 7.58 (2H, d), 7.55 (2H, dd), 7.54 |
| | (3H, dd), 7.52 (2H, s), 7.51 (4H, t), 7.5 (2H, t), 7.38 (1H, dd), 7.32 (1H, dd), 7.16 (1H, t), |
| | 7.08 (2H, d), 6.87 (1H, d), 6.69 (3H, d) |
| A184 | 8.54 (1H, s), 8.18 (1H, d), 8.16 (3H, d), 7.79 (1H, s), 7.67 (4H, d), 7.66 (1H, s), 7.62 (1H, |
| | d), 7.59 (1H, d), 7.58 (2H, d), 7.55 (1H, dd), 7.54 (2H, d), 7.52 (2H, d), 7.51 (3H, dd), 7.5 |
| | (2H, dd), 7.45 (1H, s), 7.41 (1H, t), 7.4 (1H, t), 7.2 (2H, dd), 7 (1H, dd), 6.81 (1H, t), 6.69 |
| | (2H, d), 6.63 (2H, d) |
| A188 | 8.54 (1H, s), 8.18 (1H, d), 8.16 (3H, d), 7.79 (1H, s), 7.67 (4H, d), 7.66 (1H, s), 7.62 (1H, |
| | d), 7.59 (1H, d), 7.58 (2H, d), 7.56 (1H, dd), 7.55 (1H, d), 7.54 (2H, d), 7.52 (2H, dd), 7.51 |
| | (2H, dd), 7.5 (2H, s), 7.45 (1H, t), 7.41 (1H, t), 7.4 (1H, dd), 7.2 (2H, dd), 6.81 (1H, t), 6.69 |
| | (2H, d), 6.63 (2H, d), 6.39 (1H, d) |
| A194 | 8.51 (1H, s), 8.43 (1H, d), 8.18 (1H, d), 8.16 (1H, s), 8 (2H, d), 7.88 (1H, s), 7.79 (1H, d), |
| | 7.67 (2H, d), 7.62 (1H, d), 7.59 (2H, dd), 7.58 (2H, d), 7.54 (4H, d), 7.52 (4H, dd), 7.51 |
| | (4H, dd), 7.5 (2H, s), 7.41 (3H, t), 7.13 (1H, t), 7.02 (1H, dd), 6.69 (4H, dd), 6.33 (1H, t) |
| A195 | 8.55 (2H, s), 8.54 (1H, d), 8.18 (1H, d), 8.16 (1H, s), 8 (2H, d), 7.96 (1H, s), 7.94 (1H, d), |
| | 7.92 (1H, d), 7.79 (1H, d), 7.73 (1H, dd), 7.67 (2H, d), 7.64 (1H, d), 7.62 (1H, dd), 7.59 |
| | (2H, dd), 7.58 (3H, s), 7.55 (2H, t), 7.54 (2H, t), 7.5 (2H, dd), 7.2 (2H, dd), 7.13 (1H, t), |
| | 7.02 (1H, d), 6.69 (2H, d), 6.63 (2H, d), 6.33 (1H, dd) |
| A220 | 8.55 (2H, s), 8.49 (1H, d), 8.16 (2H, d), 8.1 (1H, s), 7.67 (2H, d), 7.64 (2H, s), 7.62 (1H, d), |
| | 7.58 (2H, d), 7.55 (3H, d), 7.54 (2H, dd), 7.52 (2H, d), 7.51 (2H, d), 7.5 (2H, dd), 7.45 (1H, |
| | dd), 7.43 (1H, s), 7.41 (1H, t), 7.4 (1H, t), 7.2 (2H, dd), 6.81 (1H, dd), 6.69 (2H, t), 6.63 |
| | (2H, d), 6.33 (1H, d) |
| A223 | 8.54 (1H, s), 8.51 (1H, d), 8.49 (1H, d), 8.16 (2H, s), 8.12 (1H, d), 8.1 (1H, s), 7.67 (4H, d), |
| | 7.66 (1H, d), 7.62 (1H, d), 7.59 (1H, dd), 7.58 (2H, d), 7.57 (1H, d), 7.54 (2H, dd), 7.52 |
| | (2H, dd), 7.51 (2H, s), 7.5 (3H, t), 7.45 (1H, t), 7.41 (1H, dd), 7.2 (2H, dd), 6.81 (1H, t), |
| | 6.69 (2H, d), 6.63 (2H, d), 6.45 (1H, d) |
| A236 | 8.18 (1H, s), 8.16 (4H, d), 7.89 (1H, d), 7.79 (1H, s), 7.67 (4H, d), 7.62 (1H, s), 7.6 (1H, d), |
| | 7.58 (2H, d), 7.57 (1H, d), 7.55 (1H, dd), 7.54 (2H, d), 7.53 (1H, d), 7.5 (2H, dd), 7.45 (1H, |
| | dd), 7.4 (1H, s), 7.2 (4H, t), 6.81 (2H, t), 6.69 (2H, dd), 6.63 (4H, dd) |
| A240 | 8.51 (1H, s), 8.43 (1H, d), 8.18 (1H, d), 8.16 (3H, s), 7.89 (1H, d), 7.88 (1H, s), 7.87 (1H, |
| | d), 7.81 (1H, d), 7.79 (1H, d), 7.67 (4H, dd), 7.62 (1H, d), 7.58 (2H, d), 7.54 (2H, dd), 7.53 |
| | (1H, dd), 7.5 (2H, s), 7.45 (1H, t), 7.2 (4H, t), 6.81 (2H, dd), 6.69 (2H, dd), 6.63 (4H, t) |
| A241 | 8.51 (1H, s), 8.45 (1H, d), 8.43 (1H, d), 8.18 (1H, s), 8.16 (3H, d), 7.98 (1H, s), 7.89 (1H, |
| | d), 7.88 (1H, d), 7.81 (1H, d), 7.79 (1H, dd), 7.67 (4H, d), 7.62 (1H, d), 7.58 (2H, dd), 7.56 |
| | (1H, dd), 7.53 (1H, s), 7.52 (1H, t), 7.5 (3H, t), 7.45 (1H, dd), 7.27 (1H, dd), 7.2 (2H, t), |
| | 6.86 (1H, d), 6.81 (1H, d), 6.63 (2H, d), 6.39 (1H, dd) |
| A242 | 8.54 (1H, s), 8.18 (1H, d), 8.16 (3H, d), 7.96 (1H, s), 7.94 (1H, d), 7.89 (2H, s), 7.79 (1H, |
| | d), 7.67 (4H, d), 7.66 (1H, d), 7.62 (1H, dd), 7.58 (2H, d), 7.56 (1H, d), 7.53 (1H, dd), 7.5 |
| | (2H, dd), 7.45 (1H, s), 7.38 (1H, t), 7.32 (1H, t), 7.2 (2H, dd), 7.13 (1H, dd), 7.02 (1H, t), |
| | 6.81 (1H, d), 6.63 (2H, d), 6.39 (1H, d), 6.33 (1H, dd) |
| A256 | 8.55 (1H, s), 8.49 (1H, d), 8.18 (1H, d), 8.16 (2H, s), 8.1 (1H, d), 7.81 (1H, s), 7.72 (1H, d), |
| | 7.71 (2H, d), 7.67 (2H, d), 7.62 (1H, dd), 7.58 (2H, d), 7.55 (3H, d), 7.54 (2H, dd), 7.5 (2H, |
| | dd), 7.45 (1H, s), 7.4 (1H, t), 7.2 (4H, t), 6.81 (2H, dd), 6.69 (2H, dd), 6.63 (4H, t) |
| A257 | 8.55 (1H, s), 8.54 (1H, d), 8.49 (1H, d), 8.18 (1H, s), 8.16 (1H, d), 8.1 (1H, s), 7.87 (1H, d), |
| | 7.71 (1H, d), 7.67 (2H, d), 7.65 (2H, dd), 7.63 (1H, d), 7.62 (2H, d), 7.58 (2H, dd), 7.55 |
| | (3H, dd), 7.5 (2H, s), 7.45 (1H, t), 7.41 (1H, t), 7.38 (1H, dd), 7.28 (1H, dd), 7.2 (2H, t), |
| | 6.81 (1H, d), 6.75 (1H, d), 6.63 (2H, d), 6.58 (1H, dd), 6.39 (1H, t), 1.72 (2H, d) |
| A264 | 8.49 (1H, s), 8.16 (4H, d), 8.1 (1H, d), 7.89 (1H, s), 7.67 (4H, d), 7.62 (1H, s), 7.6 (1H, d), |
| | 7.58 (2H, d), 7.57 (1H, d), 7.55 (1H, dd), 7.54 (2H, d), 7.53 (1H, d), 7.5 (2H, dd), 7.45 (1H, |
| | dd), 7.4 (1H, s), 7.2 (4H, t), 6.81 (2H, t), 6.69 (2H, dd), 6.63 (4H, dd) |
| A265 | 8.54 (1H, s), 8.49 (1H, d), 8.16 (3H, d), 8.1 (1H, s), 7.89 (2H, d), 7.67 (4H, s), 7.66 (1H, d), |
| | 7.65 (1H, d), 7.64 (1H, d), 7.63 (1H, dd), 7.62 (1H, d), 7.58 (2H, d), 7.53 (1H, dd), 7.51 |
| | (1H, dd), 7.5 (2H, s), 7.45 (1H, t), 7.43 (1H, t), 7.39 (1H, dd), 7.38 (1H, dd), 7.32 (1H, t), |
| | 7.2 (2H, d), 6.81 (1H, d), 6.63 (2H, d), 6.33 (1H, dd) |
| A269 | 8.55 (1H, s), 8.49 (1H, d), 8.18 (1H, d), 8.16 (2H, s), 8.1 (1H, d), 7.95 (1H, s), 7.75 (1H, d), |
| | 7.71 (1H, d), 7.67 (2H, d), 7.64 (1H, dd), 7.62 (1H, d), 7.58 (2H, d), 7.55 (3H, dd), 7.54 |
| | (2H, dd), 7.5 (2H, s), 7.45 (1H, t), 7.4 (1H, t), 7.2 (4H, dd), 6.81 (2H, dd), 6.69 (2H, t), 6.63 |
| | (4H, d) |
| A275 | 8.51 (1H, s), 8.49 (1H, d), 8.16 (3H, d), 8.12 (1H, s), 8.1 (1H, d), 7.89 (1H, s), 7.87 (1H, d), |
| | 7.81 (1H, d), 7.67 (4H, d), 7.62 (1H, dd), 7.58 (2H, d), 7.57 (1H, d), 7.54 (2H, dd), 7.53 |
| | (1H, dd), 7.5 (2H, s), 7.45 (1H, t), 7.2 (4H, t), 6.81 (2H, dd), 6.69 (2H, dd), 6.63 (4H, t) |
| A281 | 8.55 (1H, s), 8.49 (1H, d), 8.18 (1H, d), 8.16 (2H, s), 8.1 (1H, d), 7.85 (1H, s), 7.81 (1H, d), |
| | 7.71 (1H, d), 7.67 (2H, d), 7.62 (1H, dd), 7.58 (2H, d), 7.55 (3H, d), 7.54 (2H, dd), 7.5 (2H, |
| | dd), 7.45 (1H, s), 7.4 (1H, t), 7.38 (1H, t), 7.2 (4H, dd), 6.81 (2H, dd), 6.69 (2H, t), 6.63 |
| | (4H, d) |
| A292 | 8.51 (1H, s), 8.49 (1H, d), 8.18 (1H, d), 8.16 (1H, s), 8.12 (1H, d), 8.1 (1H, s), 8.08 (1H, d), |
| | 7.89 (1H, d), 7.85 (1H, d), 7.81 (1H, dd), 7.68 (1H, d), 7.67 (2H, d), 7.66 (1H, dd), 7.62 |
| | (1H, dd), 7.58 (2H, s), 7.57 (1H, t), 7.55 (2H, t), 7.5 (2H, dd), 7.45 (1H, dd), 7.38 (2H, t), |
| | 7.32 (1H, d), 7.2 (2H, d), 7.13 (1H, d), 7.02 (1H, dd), 6.81 (1H, t), 6.63 (2H, d), 6.33 (1H, |
| | d) |
| A324 | 8.49 (1H, s), 8.16 (4H, d), 8.1 (1H, d), 7.87 (1H, s), 7.81 (1H, d), 7.67 (4H, s), 7.62 (1H, d), |
| | 7.58 (2H, d), 7.55 (1H, d), 7.54 (2H, dd), 7.5 (2H, d), 7.49 (1H, d), 7.45 (1H, dd), 7.42 (1H, |
| | dd), 7.4 (1H, s), 7.2 (4H, t), 6.81 (2H, t), 6.69 (2H, dd), 6.63 (4H, dd) |
| A352 | 8.49 (1H, s), 8.16 (4H, d), 8.1 (1H, d), 7.67 (4H, s), 7.62 (1H, d), 7.6 (1H, s), 7.58 (2H, d), |
| | 7.57 (1H, d), 7.55 (1H, d), 7.54 (2H, dd), 7.5 (2H, d), 7.49 (1H, d), 7.45 (1H, dd), 7.42 (1H, |
| | dd), 7.4 (1H, s), 7.2 (4H, t), 6.81 (2H, t), 6.69 (2H, dd), 6.63 (4H, dd) |
| A428 | 8.54 (1H, s), 8.49 (1H, d), 8.16 (3H, d), 8.1 (1H, s), 7.87 (1H, d), 7.81 (1H, s), 7.67 (4H, d), |
| | 7.66 (1H, d), 7.62 (1H, d), 7.59 (1H, dd), 7.58 (2H, d), 7.55 (1H, d), 7.54 (2H, dd), 7.5 (2H, |
| | dd), 7.45 (1H, s), 7.4 (1H, t), 7.2 (4H, t), 6.81 (2H, dd), 6.69 (2H, dd), 6.63 (4H, t) |
| A432 | 8.55 (2H, s), 8.49 (1H, d), 8.16 (2H, d), 8.1 (1H, s), 7.75 (1H, d), 7.67 (2H, s), 7.64 (1H, d), |
| | 7.62 (2H, d), 7.58 (2H, d), 7.55 (3H, dd), 7.54 (2H, d), 7.5 (2H, d), 7.45 (1H, dd), 7.44 (1H, |
| | dd), 7.4 (1H, s), 7.2 (4H, t), 6.81 (2H, t), 6.69 (2H, dd), 6.63 (4H, dd) |
| A442 | 8.16 (2H, s), 7.89 (1H, d), 7.83 (1H, d), 7.67 (2H, s), 7.65 (1H, d), 7.54 (2H, s), 7.53 (1H, |
| | d), 7.52 (2H, d), 7.51 (3H, d), 7.41 (1H, dd), 7.2 (2H, d), 7 (1H, d), 6.81 (1H, dd), 6.69 (2H, |
| | dd), 6.63 (2H, s) |
| A448 | 8.55 (1H, s), 8.54 (1H, d), 8.49 (1H, d), 8.18 (1H, s), 8.16 (1H, d), 8.1 (1H, s), 7.71 (1H, d), |
| | 7.67 (2H, d), 7.65 (1H, d), 7.64 (1H, dd), 7.63 (1H, d), 7.62 (1H, d), 7.55 (2H, dd), 7.54 |
| | (4H, dd), 7.52 (4H, s), 7.51 (4H, t), 7.43 (1H, t), 7.41 (2H, dd), 6.69 (4H, dd), 6.33 (1H, t) |
| A474 | 7.51 (1H, s), 7.31 (1H, d), 6.7 (1H, d) |
| A480 | 7.95 (1H, s), 7.5 (1H, d) |

TABLE 5

| Compound No. | FD-MS |
|---|---|
| A003 | m/z = 676.80 (C50H32N2O, 676.25) |
| A004 | m/z = 752.90 (C56H36N2O, 752.28) |
| A005 | m/z = 752.90 (C56H36N2O, 752.28) |
| A007 | m/z = 828.99 (C62H40N2O, 828.31) |
| A010 | m/z = 752.90 (C56H36N2O, 752.28) |
| A011 | m/z = 752.90 (C56H36N2O, 752.28) |
| A013 | m/z = 752.90 (C56H36N2O, 752.28) |
| A016 | m/z = 828.99 (C62H40N2O, 828.31) |
| A017 | m/z = 752.90 (C56H36N2O, 752.28) |
| A020 | m/z = 802.96 (C60H38N2O, 802.30) |
| A021 | m/z = 752.90 (C56H36N2O, 752.28) |
| A027 | m/z = 752.90 (C56H36N2O, 752.28) |
| A033 | m/z = 752.90 (C56H36N2O, 752.28) |
| A035 | m/z = 828.99 (C62H40N2O, 828.31) |
| A037 | m/z = 752.90 (C56H36N2O, 752.28) |
| A039 | m/z = 752.90 (C56H36N2O, 752.28) |
| A047 | m/z = 752.90 (C56H36N2O, 752.28) |
| A055 | m/z = 752.90 (C56H36N2O, 752.28) |
| A059 | m/z = 752.90 (C56H36N2O, 752.28) |
| A061 | m/z = 752.90 (C56H36N2O, 752.28) |
| A066 | m/z = 802.96 (C60H38N2O, 802.30) |
| A074 | m/z = 676.80 (C50H32N2O, 676.25) |
| A085 | m/z = 676.80 (C50H32N2O, 676.25) |
| A137 | m/z = 752.90 (C56H36N2O, 752.28) |
| A141 | m/z = 828.99 (C62H40N2O, 828.31) |
| A145 | m/z = 828.99 (C62H40N2O, 828.31) |
| A148 | m/z = 752.90 (C56H36N2O, 752.28) |
| A152 | m/z = 752.90 (C56H36N2O, 752.28) |
| A165 | m/z = 752.90 (C56H36N2O, 752.28) |
| A171 | m/z = 676.80 (C50H32N2O, 676.25) |
| A174 | m/z = 676.80 (C50H32N2O, 676.25) |
| A180 | m/z = 828.99 (C62H40N2O, 828.31) |
| A184 | m/z = 752.90 (C56H36N2O, 752.28) |
| A188 | m/z = 752.90 (C56H36N2O, 752.28) |
| A194 | m/z = 828.99 (C62H40N2O, 828.31) |
| A195 | m/z = 802.96 (C60H38N2O, 802.30) |
| A220 | m/z = 752.90 (C56H36N2O, 752.28) |
| A223 | m/z = 752.90 (C56H36N2O, 752.28) |
| A236 | m/z = 752.90 (C56H36N2O, 752.28) |
| A240 | m/z = 752.90 (C56H36N2O, 752.28) |
| A241 | m/z = 782.95 (C56H34N2OS, 782.24) |
| A242 | m/z = 2.766 (C56H34N2O2, 766.26) |
| A256 | m/z = 752.90 (C56H36N2O, 752.28) |
| A257 | m/z = 792.96 (C59H40N2O, 792.31) |
| A264 | m/z = 752.90 (C56H36N2O, 752.28) |
| A265 | m/z = 2.766 (C56H34N2O2, 766.26) |
| A269 | m/z = 752.90 (C56H36N2O, 752.28) |
| A275 | m/z = 752.90 (C56H36N2O, 752.28) |
| A281 | m/z = 752.90 (C56H36N2O, 752.28) |
| A292 | m/z = 2.766 (C56H34N2O2, 766.26) |
| A324 | m/z = 752.90 (C56H36N2O, 752.28) |
| A352 | m/z = 752.90 (C56H36N2O, 752.28) |
| A428 | m/z = 752.90 (C56H36N2O, 752.28) |
| A432 | m/z = 752.90 (C56H36N2O, 752.28) |
| A442 | m/z = 764.97 (C56H24D12N2O, 764.36) |
| A448 | m/z = 834.02 (C62H35D5N2O, 833.35) |
| A474 | m/z = 786.10 (C56H3D33N2O, 785.49) |
| A480 | m/z = 787.11 (C56H2D34N2O, 786.50) |

### Preparative Example 4

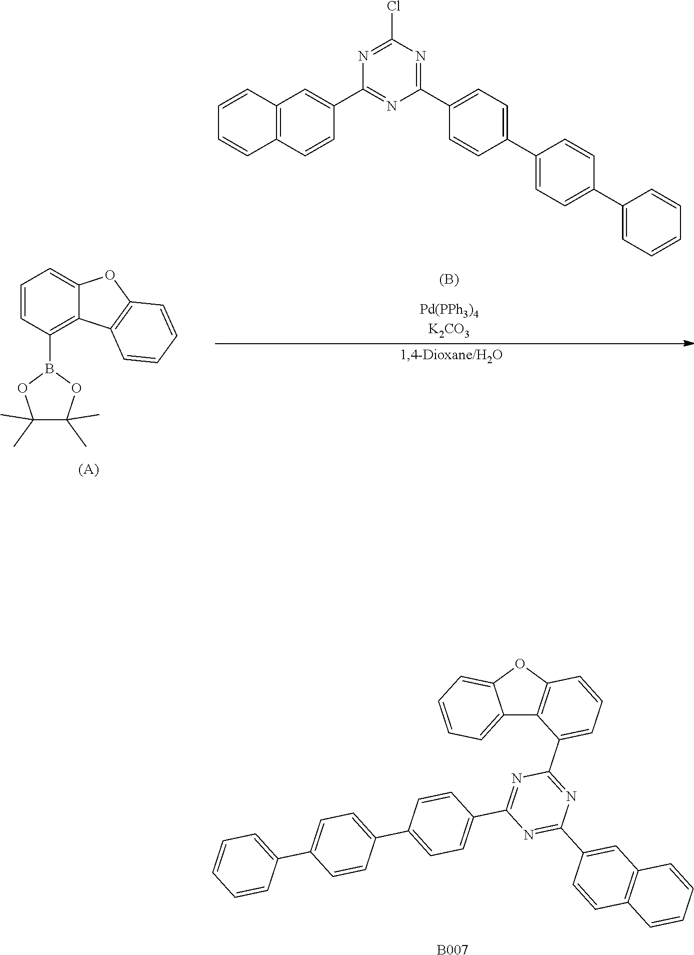

2-(dibenzo[b,d]furan-1-yl)-4,4,5,5-tetramethyl-1,3,2-dioxaborolane (A) (8 g, 27.20 mmol), 2-([1,1′: 4′,1″-phenyl]-4 yl)-4-chloro-6-(naphthalen-2-yl)-1,3,5-triazine (B) (12.50 g, 65.65 mmol), tetrakis(triphenylphosphine)palladium (0) (Pd(pph₃)₄) (1.57 g, 1.36 mmol), K₂CO₃ (11.28 g, 81.59 mmol) were dissolved in a mixed solvent of 80 mL of 1,4-dioxane and 16 mL of water, and the mixture was stirred at 110° C. for 2 hours. After cooling to room temperature, the resulting solid was filtered to obtain Compound B007 (14.59 g, 89.23%).

Compounds in Table 6 were synthesized in the same manner as above, except that (A) and (B) in Table 6 were used instead of 2-(dibenzo[b,d]furan-1-yl)-4,4,5,5-tetramethyl-1,3,2-dioxaborolane (A) and 2-([1,1′: 4′,1″-terphenyl]-4-yl)-4-chloro-6-(naphthalen-2-yl)-1,3,5-triazine (B).

TABLE 6

| Compound | | | |
|---|---|---|---|
| No. | (A) | (B) | Yield |
| B004 | 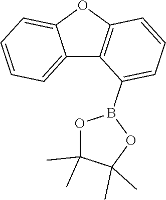 | 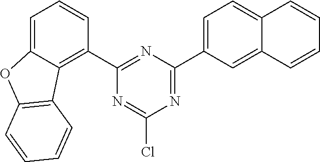 | 92% |
| B007 | 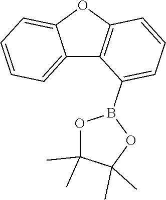 | 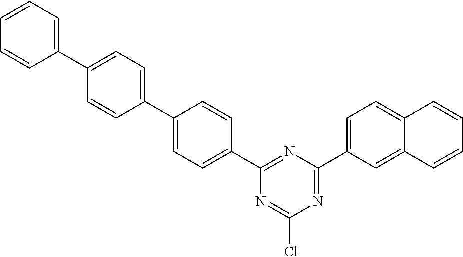 | 90% |
| B014 | 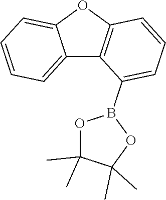 | 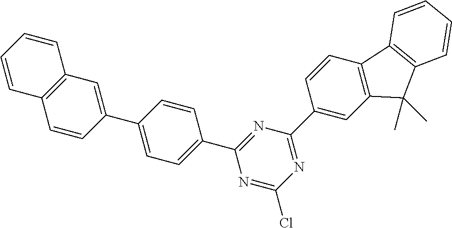 | 91% |
| B023 | 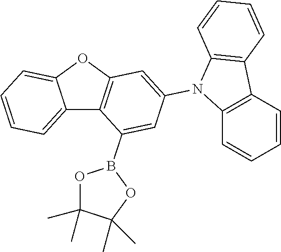 | 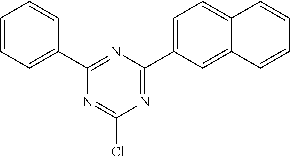 | 86% |
| B163 | 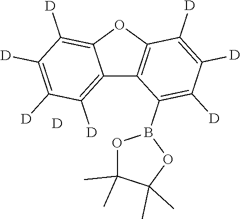 | 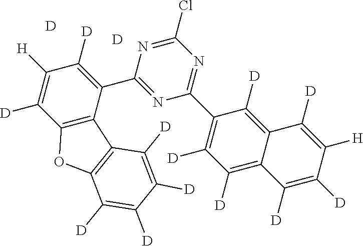 | 87% |

### Preparation Example 5

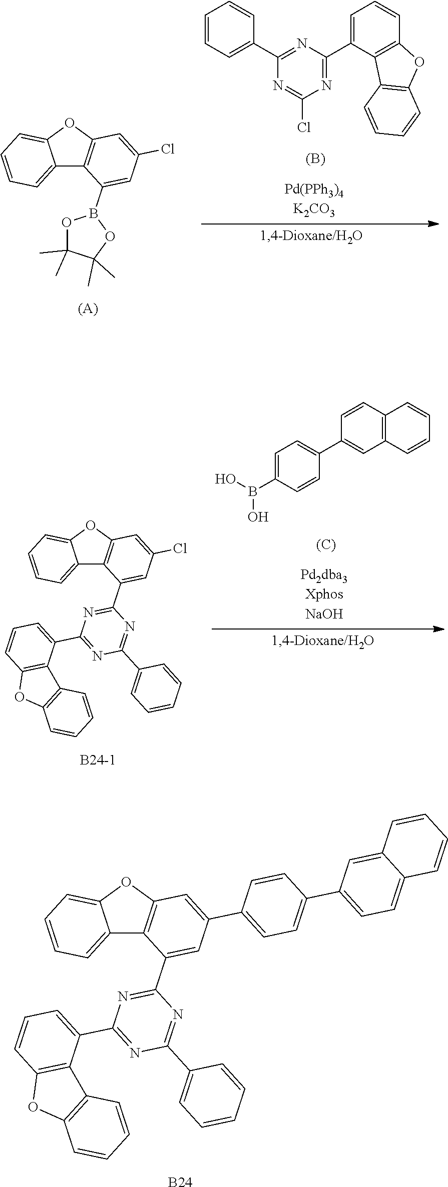

### 1) Preparation of Intermediate B44-1

2-(7-chlorodibenzo[b,d]furan-1-yl)-4,4,5,5-tetramethyl-1,3,2-dioxaborolane (A) (8 g, 24.35 mmol), 2-chloro-4-(dibenzo[b,d]furan-1-yl)-6-phenyl-1,3,5-triazine (B) (8.62 g, 23.86 mmol), tetrakis(triphenylphosphine)palladium (0) (Pd(pph₃) 4) (1.41 g, 1.22 mmol), K₂CO₃ (7.74 g, 73.04 mmol) were dissolved in a mixed solvent of 80 mL of 1,4-dioxane and 16 mL of water, and the mixture was stirred at 110° C. for 2 hours. After cooling to room temperature, the resulting solid was filtered to obtain Intermediate B44-1 (11 g, 86.23%).

### 2) Preparation of Compound B44

Intermediate B44-1 (11.0 g, 20.9 mmol), (4-(naphthalen-2-yl)phenyl) boronic acid, C) (6.25 g, 25.19 mmol), tris(dibenzylideneacetone) dipalladium (0), (Pd₂(dba)₃) (0.6 g, 1.15 mmol), 2-dicyclohexylphosphino-2′,4′,6′-triisopropylbiphenyl (XPhos) (1.0 g, 2.1 mmol), NaOH (2.52 g, 62.98 mmol) were dissolved in a mixed solvent of 110 mL of 1,4-dioxane and 22 mL of water, and the mixture was stirred at 120° C. for 2 hours. After cooling to room temperature, the resulting solid was filtered, and Compound B44 (12 g, yield: 83%) was obtained by silica purification.

Compounds in Table 7 were synthesized in the same manner as above, except that (A) of Table 7 was used instead of 2-(7-chlorodibenzo[b,d]furan-1-yl)-4,4,5,5-tetramethyl-1,3,2-dioxaborolane (A) in step 1, (B) of Table 7 was used instead of 2-chloro-4-(dibenzo[b,d]furan-1-yl)-6-phenyl-1,3,5-triazine (B), and (C) of Table 7 was used instead of (4-(naphthalen-2-yl)phenyl) boronic acid (C) in step 2.

TABLE 7

| Compound | | | | |
|---|---|---|---|---|
| No. | (A) | (B) | (C) | Yield |
| B021 | 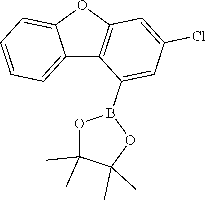 | 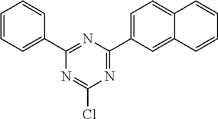 | 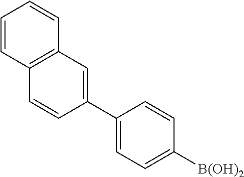 | 72% |
| B031 | 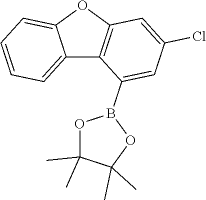 | 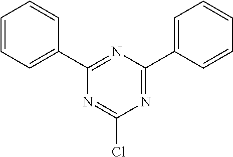 | 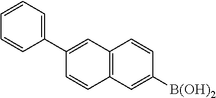 | 87% |
| B041 | 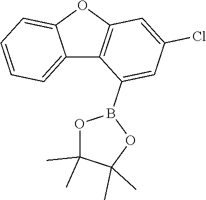 | 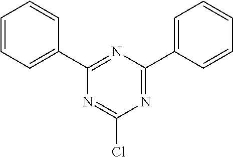 | 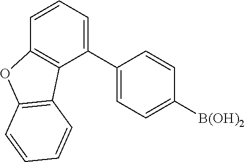 | 92% |
| B044 | 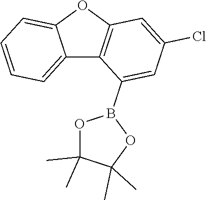 | 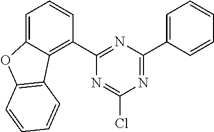 | 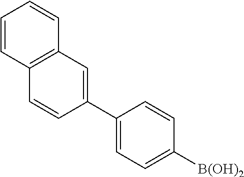 | 91% |
| B046 | 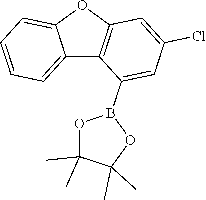 | 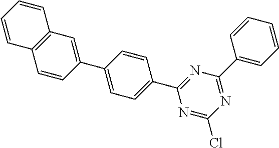 | 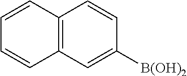 | 89% |
| B050 | 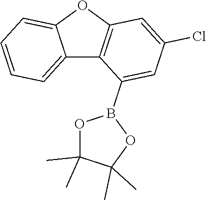 | 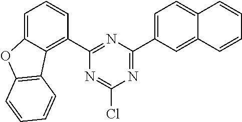 | 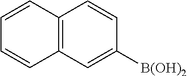 | 90% |
| B082 | 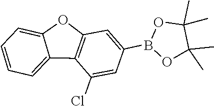 | 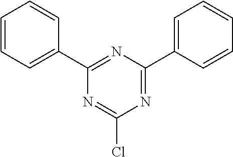 | 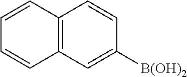 | 87% |
| B094 | 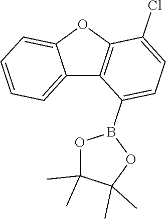 | 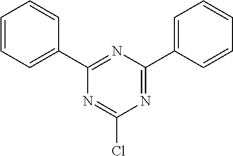 | 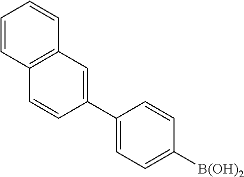 | 88% |
| B103 | 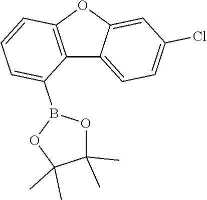 | 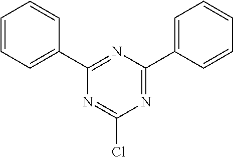 | 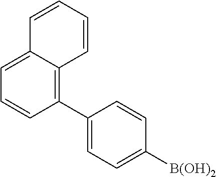 | 89% |
| B128 | 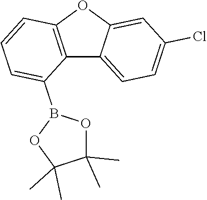 | 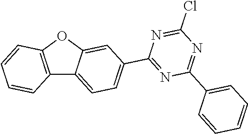 | 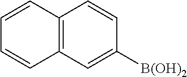 | 91% |
| B143 | 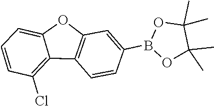 | 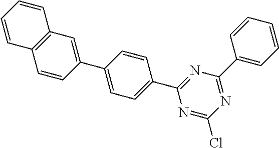 | 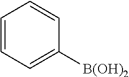 | 90% |
| B169 | 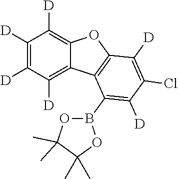 | 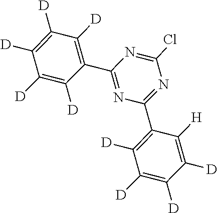 | 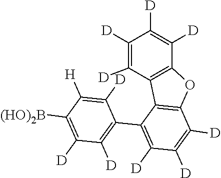 | 87% |
| B175 | 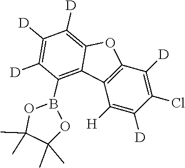 | 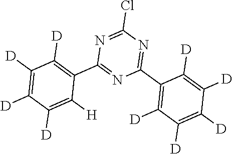 | 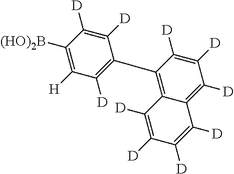 | 88% |

The synthesis results for the compounds in Tables 6 and 7 are shown in Tables 8 and 9 below. Table 8 shows the ¹H NMR (400 MHZ, CDCl₃) measurements, and Table 9 shows the field-desorption mass spectrometry (FD-MS) measurements.

TABLE 8

| Compound | |
|---|---|
| No. | ¹H NMR (400 MHz, CDCl₃) |
| B4 | 9.09 (1H, s), 8.49 (1H, d), 8 (2H, d), 7.92 (1H, s), 7.89 (2H, d), 7.75 (2H, s), 7.66 (2H, d), |
| | 7.62 (2H, d), 7.59 (2H, d), 7.44 (2H, dd), 7.38 (2H, d), 7.32 (2H, d) |
| B7 | 9.09 (1H, s), 8.49 (1H, d), 8 (2H, d), 7.92 (1H, s), 7.89 (1H, d), 7.85 (2H, s), 7.75 (1H, d), |
| | 7.66 (1H, d), 7.62 (1H, d), 7.59 (2H, dd), 7.52 (2H, d), 7.51 (2H, d), 7.44 (1H, dd), 7.41 |
| | (1H, dd), 7.38 (1H, s), 7.32 (1H, t), 7.25 (6H, t) |
| B14 | 8 (2H, s), 7.93 (1H, d), 7.92 (1H, d), 7.89 (1H, s), 7.87 (1H, d), 7.85 (2H, s), 7.77 (1H, d), |
| | 7.75 (1H, d), 7.73 (1H, d), 7.66 (1H, dd), 7.63 (1H, d), 7.62 (1H, d), 7.59 (2H, dd), 7.58 |
| | (1H, dd), 7.55 (1H, s), 7.44 (1H, t), 7.38 (2H, t), 7.32 (1H, dd), 7.28 (1H, dd), 7.25 (2H, t), |
| | 1.72 (2H, d) |
| B21 | 9.09 (1H, s), 8.49 (1H, d), 8.28 (2H, d), 8 (4H, s), 7.92 (2H, d), 7.89 (1H, s), 7.73 (1H, d), |
| | 7.66 (1H, d), 7.6 (1H, d), 7.59 (4H, dd), 7.58 (1H, d), 7.57 (1H, d), 7.51 (2H, dd), 7.41 (1H, |
| | dd), 7.38 (1H, s), 7.32 (1H, t), 7.25 (4H, t) |
| B23 | 9.09 (1H, s), 8.55 (1H, d), 8.49 (1H, d), 8.28 (2H, s), 8.12 (1H, d), 8 (2H, s), 7.94 (1H, d), |
| | 7.92 (1H, d), 7.89 (1H, d), 7.69 (1H, dd), 7.66 (2H, d), 7.63 (1H, d), 7.59 (2H, dd), 7.51 |
| | (2H, dd), 7.5 (1H, s), 7.41 (1H, t), 7.38 (1H, t), 7.33 (1H, dd), 7.32 (1H, dd), 7.29 (1H, t), |
| | 7.25 (1H, d) |
| B31 | 8.28 (4H, s), 7.92 (2H, d), 7.89 (1H, d), 7.73 (2H, s), 7.66 (1H, d), 7.6 (1H, s), 7.58 (2H, d), |
| | 7.57 (1H, d), 7.52 (2H, d), 7.51 (6H, dd), 7.41 (3H, d), 7.38 (1H, d), 7.32 (1H, dd) |
| B41 | 8.28 (4H, s), 7.89 (2H, d), 7.75 (1H, d), 7.66 (2H, s), 7.62 (1H, d), 7.6 (1H, s), 7.57 (1H, d), |
| | 7.51 (4H, d), 7.44 (1H, d), 7.41 (2H, dd), 7.38 (2H, d), 7.32 (2H, d), 7.25 (4H, dd) |
| B44 | 8.28 (2H, s), 8 (2H, d), 7.92 (1H, d), 7.89 (2H, s), 7.75 (1H, d), 7.73 (1H, s), 7.66 (2H, d), |
| | 7.62 (1H, d), 7.6 (1H, d), 7.59 (2H, dd), 7.58 (1H, d), 7.57 (1H, d), 7.51 (2H, dd), 7.44 (1H, |
| | dd), 7.41 (1H, s), 7.38 (2H, t), 7.32 (2H, t), 7.25 (4H, dd) |
| B46 | 8.28 (2H, s), 8 (4H, d), 7.92 (2H, d), 7.89 (1H, s), 7.85 (2H, d), 7.73 (2H, s), 7.66 (1H, d), |
| | 7.6 (1H, d), 7.59 (4H, d), 7.58 (2H, dd), 7.57 (1H, d), 7.51 (2H, d), 7.41 (1H, dd), 7.38 (1H, |
| | dd), 7.32 (1H, s), 7.25 (2H, t) |
| B50 | 9.09 (1H, s), 8.49 (1H, d), 8 (4H, d), 7.92 (2H, s), 7.89 (2H, d), 7.75 (1H, s), 7.73 (1H, d), |
| | 7.66 (2H, d), 7.62 (1H, d), 7.6 (1H, dd), 7.59 (4H, d), 7.58 (1H, d), 7.57 (1H, dd), 7.44 (1H, |
| | dd), 7.38 (2H, s), 7.32 (2H, t) |
| B82 | 8.28 (4H, s), 8 (2H, d), 7.92 (1H, d), 7.89 (1H, s), 7.73 (1H, d), 7.66 (1H, s), 7.6 (1H, d), |
| | 7.59 (2H, d), 7.58 (1H, d), 7.57 (1H, dd), 7.51 (4H, d), 7.41 (2H, d), 7.38 (1H, dd), 7.32 |
| | (1H, dd) |
| B94 | 8.28 (4H, s), 8 (2H, d), 7.92 (1H, d), 7.89 (1H, s), 7.87 (1H, d), 7.81 (1H, s), 7.73 (1H, d), |
| | 7.66 (1H, d), 7.59 (2H, d), 7.58 (1H, dd), 7.51 (4H, d), 7.41 (2H, d), 7.38 (1H, dd), 7.32 |
| | (1H, dd), 7.25 (4H, s) |
| B103 | 8.55 (1H, s), 8.42 (1H, d), 8.28 (4H, d), 8.08 (1H, s), 8.04 (1H, d), 7.95 (1H, s), 7.75 (2H, |
| | d), 7.64 (1H, d), 7.62 (1H, d), 7.61 (1H, dd), 7.55 (2H, d), 7.51 (4H, d), 7.44 (1H, dd), 7.41 |
| | (2H, dd), 7.25 (4H, s) |
| B128 | 8.28 (2H, s), 8 (2H, d), 7.95 (2H, d), 7.92 (1H, s), 7.89 (1H, d), 7.75 (3H, s), 7.73 (1H, d), |
| | 7.66 (1H, d), 7.64 (2H, d), 7.62 (1H, dd), 7.59 (2H, d), 7.58 (1H, d), 7.51 (2H, dd), 7.44 |
| | (1H, dd), 7.41 (1H, s), 7.38 (1H, t), 7.32 (1H, t) |
| B143 | 8.28 (2H, s), 8 (2H, d), 7.95 (1H, d), 7.92 (1H, s), 7.85 (2H, d), 7.79 (2H, s), 7.75 (2H, d), |
| | 7.73 (1H, d), 7.64 (1H, d), 7.62 (1H, dd), 7.59 (2H, d), 7.58 (1H, d), 7.51 (4H, dd), 7.44 |
| | (1H, dd), 7.41 (2H, s), 7.25 (2H, t) |
| B163 | 7.6 (1H, s), 7.56 (1H, d) |
| B169 | 7.72 (1H, s), 7.66 (1H, d) |
| B175 | 7.86 (1H, s), 7.72 (1H, d), 7.66 (1H, d) |

TABLE 9

| Compound No. | FD-MS |
|---|---|
| B4 | m/z = 2.539 (C37H21N3O2, 539.16) |
| B7 | m/z = 601.69 (C43H27N3O, 601.22) |
| B14 | m/z = 641.76 (C46H31N3O, 641.25) |
| B21 | m/z = 651.75 (C47H29N3O, 651.23) |
| B23 | m/z = 614.69 (C43H26N4O, 614.21) |
| B31 | m/z = 601.69 (C43H27N3O, 601.22) |
| B41 | m/z = 2.641 (C45H27N3O2, 641.21) |
| B44 | m/z = 2.691 (C49H29N3O2, 691.23) |
| B46 | m/z = 651.75 (C47H29N3O, 651.23) |
| B50 | m/z = 2.665 (C47H27N3O2, 665.21) |
| B82 | m/z = 525.60 (C37H23N3O, 525.18) |
| B94 | m/z = 601.69 (C43H27N3O, 601.22) |
| B103 | m/z = 601.69 (C43H27N3O, 601.22) |
| B128 | m/z = 2.615 (C43H25N3O2, 615.19) |
| B143 | m/z = 601.69 (C43H27N3O, 601.22) |
| B163 | m/z = 558.70 (C37H2D19N3O2, 558.28) |
| B169 | m/z = 666.87 (C45H2D25N3O2, 666.37) |
| B175 | m/z = 625.84 (C43H3D24N3O, 625.37) |

### Examples and Comparative Examples: Manufacture of Light-Emitting Device

A glass substrate coated with an indium tin oxide (ITO) film having a thickness of 1,500 Å was ultrasonically washed in distilled water. After completion of distilled water washing, the substrate was ultrasonically washed with solvents such as acetone, methanol, and isopropyl alcohol. After drying, the substrate was subjected to ultraviolet ozone (UVO) treatment for 5 minutes in an ultraviolet (UV) cleaner. The substrate was then transferred to a plasma cleaner (PC) and subjected to plasma treatment under vacuum to adjust the ITO work function and remove any residual film. The substrate was then transferred to a thermal evaporation device for organic vapor deposition.

A hole injection layer, 2-TNATA (4,4′,4″-tris[2-naphthyl (phenyl)amino]triphenylamine), was formed on the ITO transparent electrode (anode), and a hole transport layer, NPB (N,N′-di(1-naphthyl)-N,N′-diphenyl-(1,1′-biphenyl)-4,4′-diamine), was formed on the hole injection layer.

A emission layer was thermally vacuum-deposited on the hole transport layer as follows. Specifically, the compounds listed in Table 10 below were used alone or in combination of two thereof as a red host for the emission layer, and 3 wt % of a red phosphorescent dopant, (piq)₂Ir(acac), was doped into the red host to deposit a emission layer having a thickness of 500 Å.

Bathocuproine (BCP) was deposited on the emission layer in a thickness of 60 Å as a hole blocking layer, and Alq₃ was deposited on the hole blocking layer in a thickness of 200 Å as an electron transport layer. Lithium fluoride (LiF) was deposited on the electron transport layer in a thickness of 10 Å to form an electron injection layer.

An aluminum (Al) electrode was deposited on the electron injection layer in a thickness of 1,200 Å to form a cathode, thereby manufacturing a light-emitting device.

Meanwhile, all organic compounds required for the manufacture of the light-emitting device were purified by vacuum sublimation at 108 to 106 torr for each material and used in the manufacture of the light-emitting device.

The structures of the compounds used in the comparative examples in Tables 10 and 11 below are as follows.

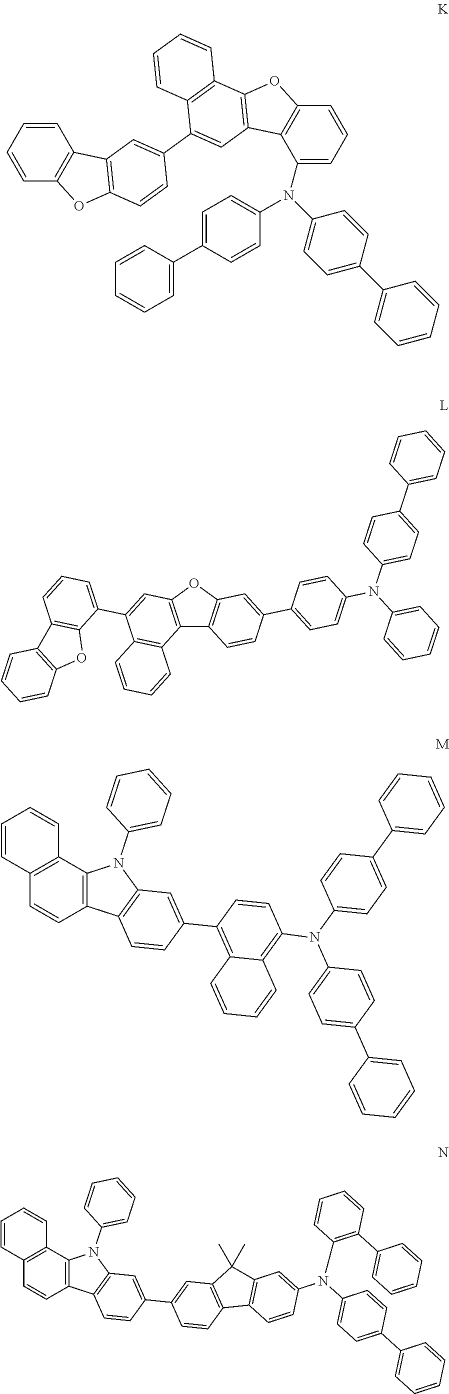 [SMILES: c1ccc(-c2ccc(N(c3ccc(-c4ccccc4)cc3)c3cccc4oc5c6ccccc6c(-c6ccc7oc8ccccc8c7c6)cc5c34)cc2)cc1 ; c1ccc(-c2ccc(N(c3ccccc3)c3ccc(-c4ccc5c(c4)oc4cc(-c6cccc7c6oc6ccccc67)c6ccccc6c45)cc3)cc2)cc1 ; c1ccc(-c2ccc(N(c3ccc(-c4ccccc4)cc3)c3ccc(-c4ccc5c6ccc7ccccc7c6n(-c6ccccc6)c5c4)c4ccccc34)cc2)cc1 ; CC1(C)c2cc(-c3ccc4c5ccc6ccccc6c5n(-c5ccccc5)c4c3)ccc2-c2ccc(N(c3ccc(-c4ccccc4)cc3)c3ccccc3-c3ccccc3)cc21]

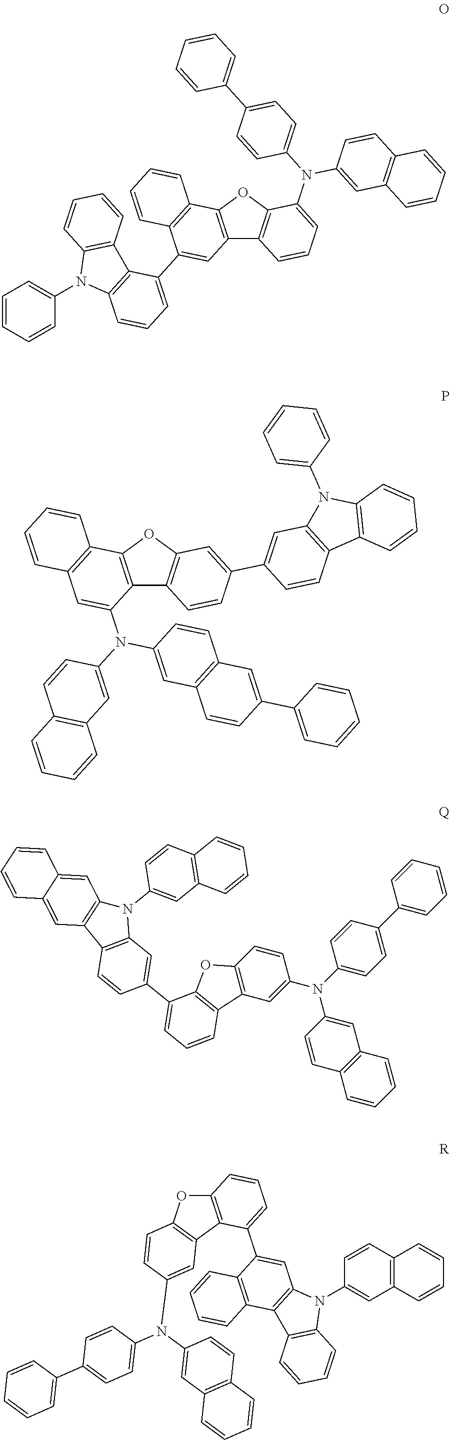 [SMILES: c1ccc(-c2ccc(N(c3ccc4ccccc4c3)c3cccc4c3oc3c5ccccc5c(-c5cccc6c5c5ccccc5n6-c5ccccc5)cc43)cc2)cc1 ; c1ccc(-c2ccc3cc(N(c4ccc5ccccc5c4)c4cc5ccccc5c5oc6cc(-c7ccc8c9ccccc9n(-c9ccccc9)c8c7)ccc6c45)ccc3c2)cc1 ; c1ccc(-c2ccc(N(c3ccc4ccccc4c3)c3ccc4oc5c(-c6ccc7c8cc9ccccc9cc8n(-c8ccc9ccccc9c8)c7c6)cccc5c4c3)cc2)cc1 ; c1ccc(-c2ccc(N(c3ccc4ccccc4c3)c3ccc4oc5cccc(-c6cc7c(c8ccccc68)c6ccccc6n7-c6ccc7ccccc7c6)c5c4c3)cc2)cc1]

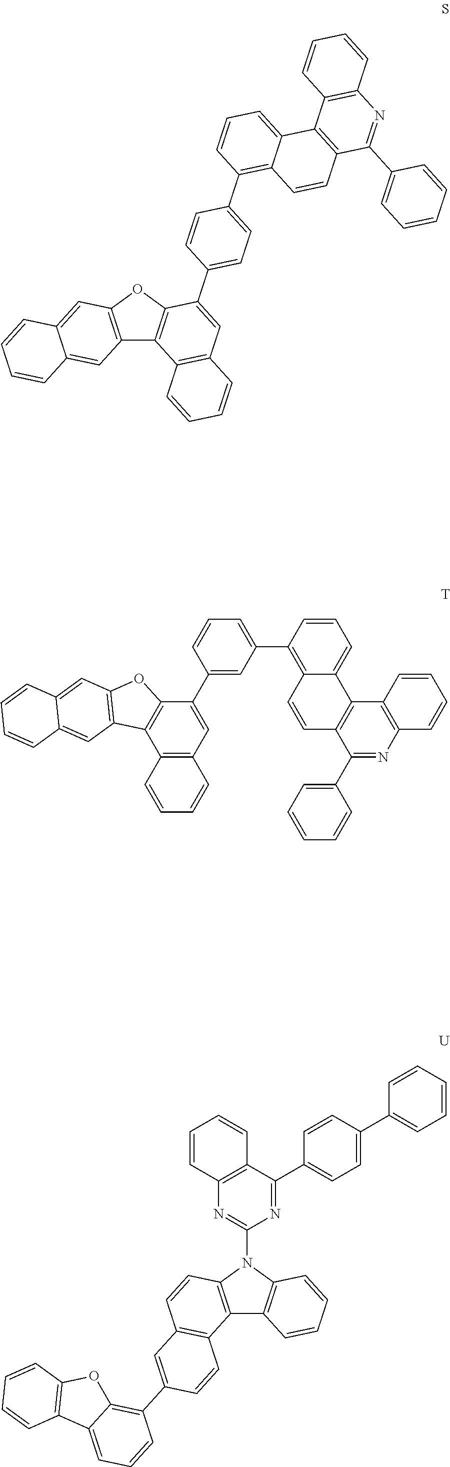 [SMILES: c1ccc(-c2nc3ccccc3c3c2ccc2c(-c4ccc(-c5cc6ccccc6c6c5oc5cc7ccccc7cc56)cc4)cccc23)cc1 ; c1ccc(-c2nc3ccccc3c3c2ccc2c(-c4cccc(-c5cc6ccccc6c6c5oc5cc7ccccc7cc56)c4)cccc23)cc1 ; c1ccc(-c2ccc(-c3nc(-n4c5ccccc5c5c6ccc(-c7cccc8c7oc7ccccc78)cc6ccc54)nc4ccccc34)cc2)cc1]

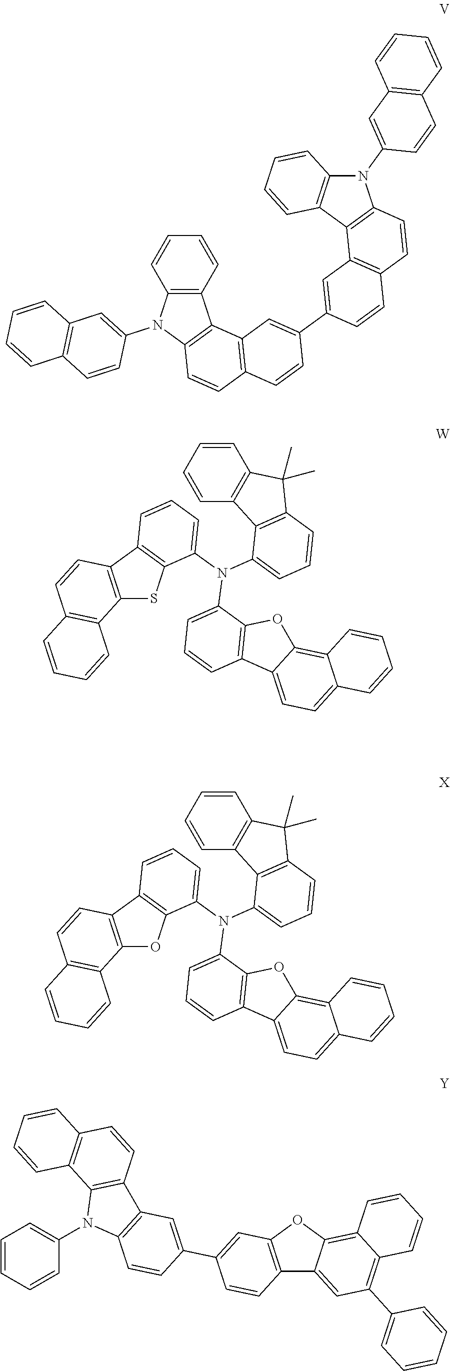 [SMILES: c1ccc2cc(-n3c4ccccc4c4c5cc(-c6ccc7ccc8c(c7c6)c6ccccc6n8-c6ccc7ccccc7c6)ccc5ccc43)ccc2c1 ; CC1(C)c2ccccc2-c2c(N(c3cccc4c3oc3c5ccccc5ccc43)c3cccc4c3sc3c5ccccc5ccc43)cccc21 ; CC1(C)c2ccccc2-c2c(N(c3cccc4c3oc3c5ccccc5ccc43)c3cccc4c3oc3c5ccccc5ccc43)cccc21 ; c1ccc(-c2cc3c4ccc(-c5ccc6c(c5)c5ccc7ccccc7c5n6-c5ccccc5)cc4oc3c3ccccc23)cc1]

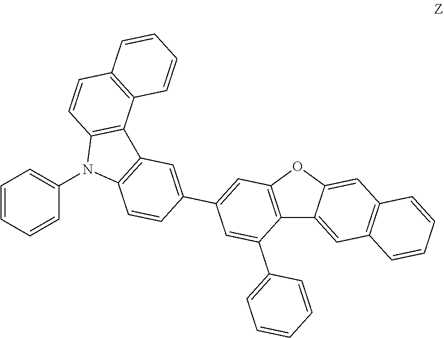 [SMILES: c1ccc(-c2cc(-c3ccc4c(c3)c3c5ccccc5ccc3n4-c3ccccc3)cc3oc4cc5ccccc5cc4c23)cc1]

TABLE 10

| | Compound | Threshold | Operating | | Color | |
|---|---|---|---|---|---|---|
| | First host | voltage | voltage | Efficiency | coordinates | Lifetime |
| No. | (P) | (Vₒₙ) | (Vₒₚ) | (cd/A) | (x, y) | (T₉₀) |
| Example 1 | A3 | 2.04 | 5.00 | 29 | (0.684, | 24 |
| | | | | | 0.316) | |
| Example 2 | A4 | 2.19 | 5.16 | 30.36 | (0.685, | 25 |
| | | | | | 0.314) | |
| Example 3 | A5 | 2.24 | 5.22 | 30.87 | (0.684, | 26 |
| | | | | | 0.315) | |
| Example 4 | A7 | 2.15 | 5.12 | 30.02 | (0.684, | 25 |
| | | | | | 0.316) | |
| Example 5 | A10 | 2.22 | 5.20 | 37.4 | (0.684, | 26 |
| | | | | | 0.316) | |
| Example 6 | A11 | 2.33 | 5.32 | 38.42 | (0.684, | 27 |
| | | | | | 0.316) | |
| Example 7 | A13 | 2.13 | 5.10 | 29.85 | (0.684, | 25 |
| | | | | | 0.316) | |
| Example 8 | A16 | 1.76 | 4.69 | 26.28 | (0.684, | 20 |
| | | | | | 0.315) | |
| Example 9 | A17 | 2.01 | 4.96 | 28.66 | (0.683, | 23 |
| | | | | | 0.317) | |
| Example 10 | A20 | 1.85 | 4.79 | 27.13 | (0.684, | 21 |
| | | | | | 0.316) | |
| Example 11 | A21 | 2.28 | 5.26 | 31.21 | (0.683, | 26 |
| | | | | | 0.318) | |
| Example 12 | A27 | 2.04 | 5.00 | 29 | (0.685, | 24 |
| | | | | | 0.314) | |
| Example 13 | A33 | 2.28 | 5.26 | 31.21 | (0.684, | 26 |
| | | | | | 0315) | |
| Example 14 | A35 | 2.22 | 5.20 | 30.7 | (0.685, | 26 |
| | | | | | 0.315) | |
| Example 15 | A37 | 2.13 | 5.10 | 29.85 | (0.683, | 25 |
| | | | | | 0.318) | |
| Example 16 | A39 | 2.04 | 5.00 | 29 | (0.684, | 24 |
| | | | | | 0315) | |
| Example 17 | A47 | 2.21 | 5.18 | 30.53 | (0.683, | 25 |
| | | | | | 0.318) | |
| Example 18 | A55 | 1.54 | 4.45 | 24.24 | (0.680, | 34 |
| | | | | | 0.319) | |
| Example 19 | A59 | 1.40 | 4.29 | 22.88 | (0.684, | 32 |
| | | | | | 0.316) | |
| Example 20 | A61 | 1.50 | 4.41 | 23.9 | (0.685, | 34 |
| | | | | | 0.315) | |
| Example 21 | A66 | 1.18 | 4.06 | 20.84 | (0.683, | 30 |
| | | | | | 0.317) | |
| Example 22 | A74 | 1.47 | 4.37 | 23.56 | (0.684, | 33 |
| | | | | | 0.315) | |
| Example 23 | A85 | 1.54 | 4.45 | 24.24 | (0.684, | 34 |
| | | | | | 0.316) | |
| Example 24 | A137 | 2.06 | 5.02 | 29.17 | (0.683, | 24 |
| | | | | | 0.318) | |
| Example 25 | A141 | 1.95 | 4.91 | 28.15 | (0.685, | 23 |
| | | | | | 0.314) | |
| Example 26 | A145 | 2.13 | 5.10 | 29.85 | (0.684, | 25 |
| | | | | | 0315) | |
| Example 27 | A148 | 2.10 | 5.06 | 29.51 | (0.685, | 24 |
| | | | | | 0.315) | |
| Example 28 | A152 | 2.19 | 5.16 | 30.36 | (0.683, | 25 |
| | | | | | 0.318) | |
| Example 29 | A165 | 1.99 | 4.94 | 28.49 | (0.684, | 23 |
| | | | | | 0.316) | |
| Example 30 | A171 | 2.01 | 4.96 | 28.66 | (0.684, | 23 |
| | | | | | 0.316) | |
| Example 31 | A174 | 2.15 | 5.12 | 30.02 | (0.685, | 25 |
| | | | | | 0.314) | |
| Example 32 | A180 | 1.92 | 4.87 | 27.81 | (0.684, | 22 |
| | | | | | 0.315) | |
| Example 33 | A184 | 2.98 | 6.03 | 17.44 | (0.684, | 42 |
| | | | | | 0.316) | |
| Example 34 | A188 | 2.91 | 5.95 | 16.76 | (0.684, | 41 |
| | | | | | 0.316) | |
| Example 35 | A194 | 2.82 | 5.85 | 15.91 | (0.684, | 40 |
| | | | | | 0.316) | |
| Example 36 | A195 | 2.96 | 6.01 | 17.27 | (0.684, | 42 |
| | | | | | 0.316) | |
| Example 37 | A220 | 3.05 | 6.11 | 18.12 | (0.684, | 43 |
| | | | | | 0.315) | |
| Example 38 | A223 | 2.91 | 5.95 | 16.76 | (0.683, | 41 |
| | | | | | 0.317) | |
| Example 39 | A236 | 2.17 | 5.14 | 30.19 | (0.684, | 25 |
| | | | | | 0.316) | |
| Example 40 | A240 | 2.13 | 5.10 | 29.85 | (0.683, | 25 |
| | | | | | 0.318) | |
| Example 41 | A241 | 2.13 | 5.10 | 28.83 | (0.685, | 25 |
| | | | | | 0.314) | |
| Example 42 | A242 | 2.12 | 5.08 | 29.34 | (0.684, | 24 |
| | | | | | 0315) | |
| Example 43 | A256 | 2.28 | 5.26 | 31.21 | (0.685, | 26 |
| | | | | | 0.315) | |
| Example 44 | A257 | 2.22 | 5.20 | 30.36 | (0.683, | 26 |
| | | | | | 0.318) | |
| Example 45 | A264 | 2.24 | 5.22 | 30.87 | (0.684, | 26 |
| | | | | | 0315) | |
| Example 46 | A265 | 2.19 | 5.16 | 30.02 | (0.683, | 25 |
| | | | | | 0.318) | |
| Example 47 | A269 | 2.28 | 5.26 | 31.21 | (0.680, | 26 |
| | | | | | 0.319) | |
| Example 48 | A275 | 2.13 | 5.10 | 29.85 | (0.684, | 25 |
| | | | | | 0.316) | |
| Example 49 | A281 | 2.31 | 5.30 | 31.55 | (0.685, | 27 |
| | | | | | 0.315) | |
| Example 50 | A292 | 2.21 | 5.18 | 30.19 | (0.683, | 25 |
| | | | | | 0.317) | |
| Example 51 | A324 | 1.52 | 4.43 | 24.07 | (0.684, | 34 |
| | | | | | 0.315) | |
| Example 52 | A352 | 1.45 | 4.35 | 23.39 | (0.685, | 33 |
| | | | | | 0.315) | |
| Example 53 | A402 | 1.52 | 4.43 | 24.07 | (0.684, | 34 |
| | | | | | 0.316) | |
| Example 54 | A428 | 2.22 | 5.20 | 17.1 | (0.683, | 26 |
| | | | | | 0.318) | |
| Example 55 | A432 | 2.30 | 5.28 | 17.78 | (0.685, | 26 |
| | | | | | 0.314) | |
| Example 56 | A442 | 2.19 | 5.16 | 30.36 | (0.685, | 55 |
| | | | | | 0.314) | |
| Example 57 | A448 | 2.22 | 5.20 | 30.7 | (0.685, | 57 |
| | | | | | 0.315) | |
| Example 58 | A474 | 2.19 | 5.16 | 30.36 | (0.685, | 70 |
| | | | | | 0.314) | |
| Example 59 | A480 | 2.31 | 5.30 | 31.55 | (0.685, | 76 |
| | | | | | 0.315) | |
| Comparative | K | 3.01 | 6.01 | 7.5 | (0.684, | 15 |
| Example 1 | | | | | 0.316) | |
| Comparative | L | 3.09 | 6.09 | 7.22 | (0.685, | 15 |
| Example 2 | | | | | 0.314) | |
| Comparative | M | 3.27 | 6.28 | 5.44 | (0.684, | 4 |
| Example 3 | | | | | 0.315) | |
| Comparative | N | 3.18 | 6.16 | 7.13 | (0.684, | 12 |
| Example 4 | | | | | 0.316) | |
| Comparative | O | 2.89 | 5.89 | 7.98 | (0.684, | 5 |
| Example 5 | | | | | 0.316) | |
| Comparative | P | 2.87 | 5.87 | 7.82 | (0.684, | 5 |
| Example 6 | | | | | 0.316) | |
| Comparative | Q | 2.81 | 5.82 | 7.79 | (0.684, | 5 |
| Example 7 | | | | | 0.316) | |
| Comparative | R | 2.84 | 5.84 | 7.64 | (0.684, | 5 |
| Example 8 | | | | | 0.315) | |
| Comparative | S | 3.98 | 6.97 | 2.64 | (0.683, | 3 |
| Example 9 | | | | | 0.317) | |
| Comparative | T | 3.92 | 6.92 | 2.57 | (0.684, | 3 |
| Example 10 | | | | | 0.316) | |
| Comparative | U | 3.7 | 6.71 | 2.7 | (0.683, | 3 |
| Example 11 | | | | | 0.318) | |
| Comparative | V | 3.45 | 6.45 | 5.37 | (0.685, | 12 |
| Example 12 | | | | | 0.314) | |
| Comparative | W | 3.33 | 6.34 | 5.64 | (0.684, | 5 |
| Example 13 | | | | | 0315) | |
| Comparative | X | 3.38 | 6.36 | 5.78 | (0.685, | 10 |
| Example 14 | | | | | 0.315) | |
| Comparative | Y | 3.61 | 6.61 | 4.95 | (0.683, | 10 |
| Example 15 | | | | | 0.318) | |
| Comparative | Z | 3.68 | 6.68 | 3.89 | (0.684, | 10 |
| Example 16 | | | | | 0315) | |

TABLE 11

| | First | Second | | Threshold | Operating | | Color | |
|---|---|---|---|---|---|---|---|---|
| | host | host | Ratio | voltage | voltage | Efficiency | coordinates | Lifetime |
| No. | (P) | (N) | (P:N) | (Von) | (Vop) | (cd/A) | (x, y) | (T90) |
| Example | A4 | B50 | 1:1 | 2.12 | 3.34 | 78.36 | (0.684, | 120 |
| 60 | | | | | | | 0.316) | |
| Example | A10 | B44 | 1:1 | 2.12 | 3.35 | 83.59 | (0.685, | 120 |
| 61 | | | | | | | 0.314) | |
| Example | A13 | B4 | 1:1 | 2.11 | 3.22 | 77.63 | (0.684, | 117 |
| 62 | | | | | | | 0.315) | |
| Example | A20 | B94 | 1:1 | 2.10 | 3.01 | 75.34 | (0.684, | 110 |
| 63 | | | | | | | 0.316) | |
| Example | A27 | B103 | 1:1 | 2.10 | 3.05 | 76.49 | (0.684, | 112 |
| 64 | | | | | | | 0.316) | |
| Example | A39 | B82 | 1:1 | 2.09 | 2.91 | 75.94 | (0.684, | 109 |
| 65 | | | | | | | 0.316) | |
| Example | A47 | B7 | 1:1 | 2.09 | 2.91 | 76.79 | (0.684, | 111 |
| 66 | | | | | | | 0.315) | |
| Example | A55 | B41 | 1:1 | 2.10 | 2.99 | 73.67 | (0.683, | 135 |
| 67 | | | | | | | 0.317) | |
| Example | A59 | B50 | 1:1 | 2.09 | 2.94 | 72.75 | (0.684, | 133 |
| 68 | | | | | | | 0.316) | |
| Example | A61 | B4 | 1:1 | 2.09 | 2.91 | 73.16 | (0.683, | 133 |
| 69 | | | | | | | 0.318) | |
| Example | A66 | B94 | 1:1 | 2.08 | 2.68 | 70.62 | (0.685, | 125 |
| 70 | | | | | | | 0.314) | |
| Example | A55 | B46 | 1:1 | 2.08 | 2.77 | 72.82 | (0.684, | 131 |
| 71 | | | | | | | 0315) | |
| Example | A59 | B31 | 1:1 | 2.07 | 2.67 | 71.70 | (0.685, | 130 |
| 72 | | | | | | | 0.315) | |
| Example | A61 | B143 | 1:1 | 2.07 | 2.62 | 72.06 | (0.683, | 127 |
| 73 | | | | | | | 0.318) | |
| Example | A66 | B23 | 1:1 | 2.05 | 2.38 | 69.47 | (0.684, | 119 |
| 74 | | | | | | | 0315) | |
| Example | A74 | B44 | 1:1 | 2.10 | 2.96 | 73.21 | (0.683, | 134 |
| 75 | | | | | | | 0.318) | |
| Example | A85 | B41 | 1:1 | 2.10 | 2.99 | 73.67 | (0.680, | 135 |
| 76 | | | | | | | 0.319) | |
| Example | A74 | B4 | 1:1 | 2.09 | 2.89 | 72.91 | (0.684, | 132 |
| 77 | | | | | | | 0.316) | |
| Example | A85 | B94 | 1:1 | 2.09 | 2.86 | 73.17 | (0.685, | 132 |
| 78 | | | | | | | 0.315) | |
| Example | A74 | B128 | 1:1 | 2.08 | 2.72 | 72.26 | (0.683, | 129 |
| 79 | | | | | | | 0.317) | |
| Example | A74 | B82 | 1:1 | 2.07 | 2.62 | 71.86 | (0.684, | 127 |
| 80 | | | | | | | 0.316) | |
| Example | A85 | B14 | 1:1 | 2.06 | 2.55 | 71.97 | (0.683, | 126 |
| 81 | | | | | | | 0.318) | |
| Example | A137 | B50 | 1:1 | 2.12 | 3.28 | 77.47 | (0.685, | 117 |
| 82 | | | | | | | 0.314) | |
| Example | A141 | B44 | 1:1 | 2.11 | 3.21 | 76.65 | (0.684, | 115 |
| 83 | | | | | | | 0315) | |
| Example | A145 | B4 | 1:1 | 2.11 | 3.22 | 77.63 | (0.685, | 117 |
| 84 | | | | | | | 0.315) | |
| Example | A152 | B94 | 1:1 | 2.11 | 3.19 | 77.76 | (0.683, | 117 |
| 85 | | | | | | | 0.318) | |
| Example | A165 | B21 | 1:1 | 2.09 | 2.95 | 75.81 | (0.684, | 110 |
| 86 | | | | | | | 0.316) | |
| Example | A174 | B82 | 1:1 | 2.09 | 2.96 | 76.70 | (0.685, | 111 |
| 87 | | | | | | | 0.314) | |
| Example | A180 | B23 | 1:1 | 2.08 | 2.76 | 74.70 | (0.684, | 105 |
| 88 | | | | | | | 0.315) | |
| Example | A194 | B50 | 1:1 | 2.14 | 3.66 | 67.52 | (0.684, | 147 |
| 89 | | | | | | | 0.316) | |
| Example | A195 | B41 | 1:1 | 2.14 | 3.71 | 68.44 | (0.684, | 149 |
| 90 | | | | | | | 0.316) | |
| Example | A220 | B4 | 1:1 | 2.14 | 3.69 | 68.83 | (0.684, | 150 |
| 91 | | | | | | | 0.316) | |
| Example | A194 | B94 | 1:1 | 2.13 | 3.51 | 66.92 | (0.684, | 144 |
| 92 | | | | | | | 0.316) | |
| Example | A195 | B46 | 1:1 | 2.13 | 3.49 | 67.59 | (0.684, | 145 |
| 93 | | | | | | | 0.315) | |
| Example | A194 | B143 | 1:1 | 2.11 | 3.29 | 66.07 | (0.684, | 139 |
| 94 | | | | | | | 0.316) | |
| Example | A220 | B7 | 1:1 | 2.12 | 3.34 | 67.48 | (0.683, | 142 |
| 95 | | | | | | | 0.318) | |
| Example | A184 | B50 | 1:1 | 2.15 | 3.74 | 68.67 | (0.685, | 150 |
| 96 | | | | | | | 0.314) | |
| Example | A188 | B41 | 1:1 | 2.14 | 3.68 | 68.06 | (0.684, | 148 |
| 97 | | | | | | | 0315) | |
| Example | A223 | B4 | 1:1 | 2.14 | 3.61 | 67.81 | (0.685, | 147 |
| 98 | | | | | | | 0.315) | |
| Example | A184 | B94 | 1:1 | 2.14 | 3.59 | 68.07 | (0.683, | 147 |
| 99 | | | | | | | 0.318) | |
| Example | A188 | B103 | 1:1 | 2.13 | 3.49 | 67.31 | (0.684, | 144 |
| 100 | | | | | | | 0315) | |
| Example | A223 | B82 | 1:1 | 2.12 | 3.35 | 66.76 | (0.680, | 141 |
| 101 | | | | | | | 0.319) | |
| Example | A184 | B14 | 1:1 | 2.11 | 3.28 | 66.87 | (0.684, | 140 |
| 102 | | | | | | | 0.316) | |
| Example | A236 | B41 | 1:1 | 2.12 | 3.31 | 78.13 | (0.685, | 119 |
| 103 | | | | | | | 0.315) | |
| Example | A240 | B44 | 1:1 | 2.12 | 3.30 | 77.93 | (0.683, | 119 |
| 104 | | | | | | | 0.317) | |
| Example | A256 | B4 | 1:1 | 2.12 | 3.30 | 78.65 | (0.684, | 120 |
| 105 | | | | | | | 0.315) | |
| Example | A264 | B94 | 1:1 | 2.11 | 3.21 | 78.14 | (0.685, | 118 |
| 106 | | | | | | | 0.315) | |
| Example | A269 | B46 | 1:1 | 2.11 | 3.14 | 78.05 | (0.684, | 116 |
| 107 | | | | | | | 0.316) | |
| Example | A275 | B31 | 1:1 | 2.10 | 3.04 | 76.93 | (0.683, | 116 |
| 108 | | | | | | | 0.318) | |
| Example | A281 | B82 | 1:1 | 2.10 | 3.05 | 77.85 | (0.685, | 115 |
| 109 | | | | | | | 0.314) | |
| Example | A281 | B7 | 1:1 | 2.09 | 2.97 | 77.55 | (0.684, | 113 |
| 110 | | | | | | | 0315) | |
| Example | A241 | B41 | 1:1 | 2.12 | 3.29 | 77.11 | (0.684, | 118 |
| 111 | | | | | | | 0.316) | |
| Example | A242 | B41 | 1:1 | 2.12 | 3.28 | 77.49 | (0.685, | 118 |
| 112 | | | | | | | 0.314) | |
| Example | A257 | B4 | 1:1 | 2.12 | 3.27 | 78.01 | (0.684, | 119 |
| 113 | | | | | | | 0.315) | |
| Example | A265 | B94 | 1:1 | 2.11 | 3.19 | 77.50 | (0.684, | 117 |
| 114 | | | | | | | 0.316) | |
| Example | A292 | B31 | 1:1 | 2.10 | 3.08 | 77.18 | (0.684, | 117 |
| 115 | | | | | | | 0.316) | |
| Example | A265 | B143 | 1:1 | 2.09 | 2.97 | 76.65 | (0.684, | 112 |
| 116 | | | | | | | 0.316) | |
| Example | A292 | B23 | 1:1 | 2.09 | 2.90 | 76.48 | (0.684, | 111 |
| 117 | | | | | | | 0.315) | |
| Example | A324 | B44 | 1:1 | 2.10 | 2.99 | 73.59 | (0.683, | 135 |
| 118 | | | | | | | 0.317) | |
| Example | A352 | B50 | 1:1 | 2.10 | 2.97 | 73.13 | (0.684, | 134 |
| 119 | | | | | | | 0.316) | |
| Example | A324 | B4 | 1:1 | 2.09 | 2.91 | 73.29 | (0.683, | 134 |
| 120 | | | | | | | 0.318) | |
| Example | A352 | B94 | 1:1 | 2.08 | 2.81 | 72.53 | (0.685, | 131 |
| 121 | | | | | | | 0.314) | |
| Example | A352 | B21 | 1:1 | 2.07 | 2.67 | 71.98 | (0.685, | 128 |
| 122 | | | | | | | 0.315) | |
| Example | A324 | B82 | 1:1 | 2.07 | 2.65 | 72.24 | (0.684, | 128 |
| 123 | | | | | | | 0.316) | |
| Example | A352 | B14 | 1:1 | 2.06 | 2.51 | 71.33 | (0.685, | 124 |
| 124 | | | | | | | 0.314) | |
| Example | A428 | B50 | 1:1 | 2.12 | 3.36 | 68.41 | (0.684, | 121 |
| 125 | | | | | | | 0.315) | |
| Example | A432 | B50 | 1:1 | 2.12 | 3.40 | 68.92 | (0.684, | 122 |
| 126 | | | | | | | 0.316) | |
| Example | A428 | B4 | 1:1 | 2.12 | 3.27 | 68.06 | (0.684, | 119 |
| 127 | | | | | | | 0.316) | |
| Example | A432 | B94 | 1:1 | 2.11 | 3.24 | 68.32 | (0.684, | 119 |
| 128 | | | | | | | 0.316) | |
| Example | A428 | B103 | 1:1 | 2.11 | 3.14 | 67.56 | (0.684, | 116 |
| 129 | | | | | | | 0.316) | |
| Example | A432 | B31 | 1:1 | 2.10 | 3.13 | 67.87 | (0.684, | 119 |
| 130 | | | | | | | 0.315) | |
| Example | A428 | B82 | 1:1 | 2.10 | 3.00 | 67.01 | (0.683, | 113 |
| 131 | | | | | | | 0.317) | |
| Example | A432 | B14 | 1:1 | 2.09 | 2.93 | 67.12 | (0.684, | 112 |
| 132 | | | | | | | 0.316) | |
| Example | A442 | B50 | 1:1 | 2.12 | 3.34 | 78.36 | (0.684, | 140 |
| 133 | | | | | | | 0.316) | |
| Example | A474 | B50 | 1:1 | 2.12 | 3.34 | 78.36 | (0.684, | 156 |
| 134 | | | | | | | 0.316) | |
| Example | A480 | B7 | 1:1 | 2.09 | 2.97 | 77.55 | (0.684, | 147 |
| 135 | | | | | | | 0315) | |
| Example | A10 | B169 | 1:1 | 2.12 | 3.33 | 83.54 | (0.684, | 156 |
| 136 | | | | | | | 0.316) | |
| Example | A10 | B50 | 1:1 | 2.12 | 3.36 | 83.64 | (0.683, | 121 |
| 137 | | | | | | | 0.318) | |
| Example | A10 | B50 | 2:1 | 2.12 | 3.29 | 83.02 | (0.685, | 123 |
| 138 | | | | | | | 0.314) | |
| Example | A10 | B50 | 1.5:1 | 2.12 | 3.33 | 83.70 | (0.684, | 123 |
| 139 | | | | | | | 0315) | |
| Example | A10 | B50 | 1:1.5 | 2.12 | 3.40 | 81.39 | (0.685, | 129 |
| 140 | | | | | | | 0.315) | |
| Example | A10 | B44 | 1:1 | 2.12 | 3.31 | 82.59 | (0.683, | 124 |
| 141 | | | | | | | 0.318) | |
| Example | A10 | B44 | 2:1 | 2.12 | 3.29 | 82.96 | (0.684, | 122 |
| 142 | | | | | | | 0315) | |
| Example | A10 | B44 | 1.5:1 | 2.12 | 3.32 | 83.62 | (0.683, | 122 |
| 143 | | | | | | | 0.318) | |
| Example | A10 | B44 | 1:1.5 | 2.12 | 3.38 | 81.27 | (0.680, | 129 |
| 144 | | | | | | | 0.319) | |
| Example | A10 | B41 | 1:1 | 2.12 | 3.33 | 83.54 | (0.684, | 120 |
| 145 | | | | | | | 0.316) | |
| Example | A10 | B41 | 1.5:1 | 2.12 | 3.31 | 83.54 | (0.685, | 122 |
| 146 | | | | | | | 0.315) | |
| Example | A11 | B50 | 1:1 | 2.13 | 3.41 | 84.40 | (0.683, | 123 |
| 147 | | | | | | | 0.317) | |
| Example | A11 | B50 | 1.5:1 | 2.12 | 3.41 | 84.41 | (0.684, | 124 |
| 148 | | | | | | | 0.315) | |
| Example | A281 | B50 | 1:1 | 2.12 | 3.40 | 79.25 | (0.684, | 123 |
| 149 | | | | | | | 0.316) | |
| Example | A11 | B14 | 1:1 | 2.09 | 2.95 | 82.60 | (0.683, | 113 |
| 150 | | | | | | | 0.318) | |
| Example | A11 | B7 | 1:1 | 2.09 | 2.98 | 82.70 | (0.685, | 113 |
| 151 | | | | | | | 0.314) | |
| Example | A220 | B50 | 1:1 | 2.15 | 3.78 | 69.18 | (0.684, | 152 |
| 152 | | | | | | | 0315) | |
| Example | A220 | B50 | 2:1 | 2.15 | 3.85 | 70.30 | (0.685, | 147 |
| 153 | | | | | | | 0.315) | |
| Example | A220 | B50 | 1.5:1 | 2.16 | 3.92 | 70.20 | (0.683, | 147 |
| 154 | | | | | | | 0.318) | |
| Example | A220 | B50 | 1:1.5 | 2.15 | 3.73 | 73.67 | (0.684, | 146 |
| 155 | | | | | | | 0.316) | |
| Example | A220 | B50 | 1:2 | 2.14 | 3.69 | 74.54 | (0.684, | 145 |
| 156 | | | | | | | 0.316) | |
| Example | A184 | B23 | 1:1 | 2.11 | 3.29 | 66.92 | (0.685, | 140 |
| 157 | | | | | | | 0.314) | |

TABLE 12

| | First | Second | | Threshold | Operating | | Color | |
|---|---|---|---|---|---|---|---|---|
| | host | host | Ratio | voltage | voltage | Efficiency | coordinates | Lifetime |
| No. | (P) | (N) | (P:N) | (Von) | (Vop) | (cd/A) | (x, y) | (T90) |
| Comparative | K | B50 | 1:1 | 2.35 | 3.73 | 59.26 | (0.684, | 87 |
| Example 17 | | | | | | | 0.316) | |
| Comparative | L | B94 | 1:1 | 2.34 | 3.62 | 56.18 | (0.685, | 84 |
| Example 18 | | | | | | | 0.314) | |
| Comparative | K | B103 | 1:1 | 2.33 | 3.51 | 56.03 | (0.684, | 82 |
| Example 19 | | | | | | | 0.315) | |
| Comparative | L | B82 | 1:1 | 2.32 | 3.41 | 53.14 | (0.684, | 79 |
| Example 20 | | | | | | | 0.316) | |
| Comparative | K | B7 | 1:1 | 2.32 | 3.30 | 52.80 | (0.684, | 77 |
| Example 21 | | | | | | | 0.316) | |
| Comparative | M | B44 | 1:1 | 2.36 | 3.84 | 53.20 | (0.684, | 65 |
| Example 22 | | | | | | | 0.316) | |
| Comparative | M | B94 | 1:1 | 2.35 | 3.70 | 51.11 | (0.684, | 61 |
| Example 23 | | | | | | | 0.316) | |
| Comparative | M | B143 | 1:1 | 2.33 | 3.49 | 47.88 | (0.683, | 56 |
| Example 24 | | | | | | | 0.317) | |
| Comparative | M | B23 | 1:1 | 2.32 | 3.41 | 46.74 | (0.684, | 54 |
| Example 25 | | | | | | | 0.316) | |
| Comparative | N | B41 | 1:1 | 2.35 | 3.78 | 57.83 | (0.683, | 81 |
| Example 26 | | | | | | | 0.318) | |
| Comparative | N | B94 | 1:1 | 2.34 | 3.65 | 55.93 | (0.685, | 78 |
| Example 27 | | | | | | | 0.314) | |
| Comparative | N | B46 | 1:1 | 2.34 | 3.56 | 54.60 | (0.684, | 75 |
| Example 28 | | | | | | | 0315) | |
| Comparative | N | B82 | 1:1 | 2.33 | 3.44 | 52.89 | (0.685, | 72 |
| Example 29 | | | | | | | 0.315) | |
| Comparative | N | B14 | 1:1 | 2.32 | 3.34 | 51.37 | (0.683, | 70 |
| Example 30 | | | | | | | 0.318) | |
| Comparative | O | B4 | 1:1 | 2.34 | 3.59 | 59.30 | (0.684, | 65 |
| Example 31 | | | | | | | 0315) | |
| Comparative | P | B94 | 1:1 | 2.33 | 3.51 | 57.89 | (0.683, | 63 |
| Example 32 | | | | | | | 0.318) | |
| Comparative | O | B128 | 1:1 | 2.33 | 3.42 | 56.83 | (0.680, | 61 |
| Example 33 | | | | | | | 0.319) | |
| Comparative | P | B143 | 1:1 | 2.32 | 3.30 | 54.66 | (0.684, | 58 |
| Example 34 | | | | | | | 0.316) | |
| Comparative | O | B7 | 1:1 | 2.31 | 3.24 | 54.17 | (0.685, | 56 |
| Example 35 | | | | | | | 0.315) | |
| Comparative | Q | B50 | 1:1 | 2.34 | 3.64 | 60.09 | (0.683, | 67 |
| Example 36 | | | | | | | 0.317) | |
| Comparative | R | B94 | 1:1 | 2.33 | 3.50 | 57.38 | (0.684, | 63 |
| Example 37 | | | | | | | 0.315) | |
| Comparative | Q | B31 | 1:1 | 2.32 | 3.38 | 56.10 | (0.684, | 64 |
| Example 38 | | | | | | | 0.316) | |
| Comparative | R | B82 | 1:1 | 2.32 | 3.30 | 54.34 | (0.683, | 58 |
| Example 39 | | | | | | | 0.318) | |
| Comparative | Q | B23 | 1:1 | 2.31 | 3.20 | 53.44 | (0.685, | 56 |
| Example 40 | | | | | | | 0.314) | |
| Comparative | S | B44 | 1:1 | 2.38 | 4.16 | 45.22 | (0.684, | 63 |
| Example 41 | | | | | | | 0315) | |
| Comparative | T | B94 | 1:1 | 2.37 | 4.00 | 42.93 | (0.685, | 59 |
| Example 42 | | | | | | | 0.315) | |
| Comparative | T | B143 | 1:1 | 2.35 | 3.78 | 39.70 | (0.684, | 54 |
| Example 43 | | | | | | | 0.316) | |
| Comparative | S | B14 | 1:1 | 2.35 | 3.71 | 38.57 | (0.684, | 52 |
| Example 44 | | | | | | | 0.316) | |
| Comparative | U | B41 | 1:1 | 2.37 | 4.03 | 45.20 | (0.685, | 62 |
| Example 45 | | | | | | | 0.314) | |
| Comparative | U | B94 | 1:1 | 2.36 | 3.90 | 43.30 | (0.684, | 59 |
| Example 46 | | | | | | | 0.315) | |
| Comparative | U | B21 | 1:1 | 2.35 | 3.76 | 41.21 | (0.684, | 56 |
| Example 47 | | | | | | | 0.316) | |
| Comparative | U | B82 | 1:1 | 2.35 | 3.70 | 40.26 | (0.684, | 54 |
| Example 48 | | | | | | | 0.316) | |
| Comparative | U | B7 | 1:1 | 2.34 | 3.62 | 39.12 | (0.684, | 52 |
| Example 49 | | | | | | | 0.316) | |
| Comparative | V | B4 | 1:1 | 2.36 | 3.85 | 51.86 | (0.684, | 79 |
| Example 50 | | | | | | | 0.316) | |
| Comparative | V | B94 | 1:1 | 2.35 | 3.78 | 50.91 | (0.684, | 78 |
| Example 51 | | | | | | | 0.315) | |
| Comparative | V | B103 | 1:1 | 2.35 | 3.72 | 49.96 | (0.683, | 76 |
| Example 52 | | | | | | | 0.317) | |
| Comparative | V | B143 | 1:1 | 2.34 | 3.56 | 47.68 | (0.684, | 72 |
| Example 53 | | | | | | | 0.316) | |
| Comparative | V | B23 | 1:1 | 2.33 | 3.49 | 46.54 | (0.683, | 70 |
| Example 54 | | | | | | | 0.318) | |
| Comparative | W | B50 | 1:1 | 2.36 | 3.88 | 53.96 | (0.685, | 67 |
| Example 55 | | | | | | | 0.314) | |
| Comparative | X | B94 | 1:1 | 2.35 | 3.74 | 52.08 | (0.684, | 73 |
| Example 56 | | | | | | | 0315) | |
| Comparative | X | B82 | 1:1 | 2.33 | 3.53 | 49.04 | (0.683, | 68 |
| Example 57 | | | | | | | 0.318) | |
| Comparative | W | B14 | 1:1 | 2.33 | 3.42 | 47.12 | (0.684, | 56 |
| Example 58 | | | | | | | 0315) | |
| Comparative | Y | B44 | 1:1 | 2.37 | 4.00 | 51.81 | (0.683, | 77 |
| Example 59 | | | | | | | 0.318) | |
| Comparative | Z | B94 | 1:1 | 2.36 | 3.89 | 46.69 | (0.680, | 73 |
| Example 60 | | | | | | | 0.319) | |
| Comparative | Y | B46 | 1:1 | 2.35 | 3.77 | 48.39 | (0.684, | 71 |
| Example 61 | | | | | | | 0.316) | |
| Comparative | Z | B143 | 1:1 | 2.34 | 3.67 | 43.46 | (0.685, | 68 |
| Example 62 | | | | | | | 0.315) | |
| Comparative | Y | B7 | 1:1 | 2.34 | 3.57 | 45.54 | (0.683, | 67 |
| Example 63 | | | | | | | 0.317) | |
| Comparative | O | B50 | 1:1 | 2.34 | 3.68 | 60.63 | (0.684, | 67 |
| Example 64 | | | | | | | 0.315) | |
| Comparative | O | B50 | 2:1 | 2.35 | 3.71 | 50.28 | (0.685, | 50 |
| Example 65 | | | | | | | 0.315) | |
| Comparative | O | B50 | 1.5:1 | 2.35 | 3.70 | 54.21 | (0.684, | 57 |
| Example 66 | | | | | | | 0.316) | |
| Comparative | O | B50 | 1:1.5 | 2.34 | 3.65 | 63.52 | (0.683, | 69 |
| Example 67 | | | | | | | 0.318) | |
| Comparative | O | B50 | 1:2 | 2.34 | 3.63 | 60.19 | (0.685, | 66 |
| Example 68 | | | | | | | 0.314) | |
| Comparative | O | B44 | 1:1 | 2.34 | 3.66 | 60.44 | (0.684, | 67 |
| Example 69 | | | | | | | 0315) | |
| Comparative | P | B14 | 1:1 | 2.31 | 3.21 | 53.33 | (0.684, | 56 |
| Example 70 | | | | | | | 0.316) | |
| Comparative | P | B7 | 1:1 | 2.31 | 3.23 | 53.71 | (0.685, | 56 |
| Example 71 | | | | | | | 0.314) | |
| Comparative | K | B50 | 1:1 | 2.35 | 3.73 | 59.26 | (0.684, | 87 |
| Example 72 | | | | | | | 0.315) | |
| Comparative | K | B50 | 2:1 | 2.35 | 3.79 | 48.48 | (0.684, | 77 |
| Example 73 | | | | | | | 0.316) | |
| Comparative | K | B50 | 1.5:1 | 2.35 | 3.77 | 52.57 | (0.684, | 81 |
| Example 74 | | | | | | | 0.316) | |
| Comparative | K | B50 | 1:1.5 | 2.35 | 3.70 | 62.49 | (0.684, | 83 |
| Example 75 | | | | | | | 0.316) | |
| Comparative | K | B50 | 1:2 | 2.34 | 3.66 | 59.43 | (0.684, | 76 |
| Example 76 | | | | | | | 0.316) | |
| Comparative | K | B44 | 1:1 | 2.35 | 3.72 | 59.07 | (0.684, | 87 |
| Example 77 | | | | | | | 0.315) | |
| Comparative | L | B14 | 1:1 | 2.32 | 3.31 | 51.62 | (0.683, | 76 |
| Example 78 | | | | | | | 0.317) | |
| Comparative | L | B7 | 1:1 | 2.32 | 3.33 | 52.00 | (0.684, | 77 |
| Example 79 | | | | | | | 0.316) | |
| Comparative | L | B7 | 2:1 | 2.33 | 3.55 | 43.16 | (0.683, | 70 |
| Example 80 | | | | | | | 0.318) | |
| Comparative | L | B7 | 1.5:1 | 2.33 | 3.46 | 46.44 | (0.685, | 72 |
| Example 81 | | | | | | | 0.314) | |
| Comparative | L | B7 | 1:1.5 | 2.31 | 3.20 | 54.54 | (0.684, | 72 |
| Example 82 | | | | | | | 0315) | |
| Comparative | L | B7 | 1:2 | 2.30 | 3.11 | 51.81 | (0.685, | 65 |
| Example 83 | | | | | | | 0.315) | |

Compounds K, L, O and P in the comparative examples have two substituents, dibenzofuran and arylamine. The dibenzofuran substituent has lower hole transport properties than the naphthobenzocarbazole in Compounds A011 and A220, resulting in relatively low hole transport properties of the compounds. This property is believed to have caused a charge imbalance in the emission layer, resulting in reduced lifetime and efficiency, as seen in Comparative Examples 1, 2, 5 and 6.

Compounds M, N, Q, and R in the comparative examples have two substituents centered around different core structures. Although naphthobenzocarbazole substituents possess high hole transport properties, Compounds M and N of the comparative examples exhibited a more severe conjugation breakdown than the core containing naphthobenzofuran, as in Compound A35, leading to reduced lifetime and efficiency, as seen in Comparative Examples 3, 4, 7 and 8.

Compounds S and T of the comparative examples, like Compound A194, possess naphthobenzofuran but are substituted with substituents having significantly lower hole transport properties. This leads to a lower charge balance than Compound A194, which is believed to have resulted in reduced lifetime and efficiency, as seen in Comparative Examples 9 and 10.

Compounds U and V of the comparative examples include naphthobenzocarbazole substituents with superior hole transport properties. However, as previously mentioned, the intramolecular conjugation is shorter than that of the compounds of the examples, resulting in poor electron transfer performance, as seen in Comparative Examples 11 and 12.

Compounds W and X of the comparative examples are expected to exhibit excellent transfer performance due to the expanded conjugation of the naphthobenzofuran structure, which is doubly substituted. However, due to the absence of a substituent with excellent hole transport properties of naphthobenzocarbazole, charge balance is impaired, leading to performance degradation as seen in Comparative Examples 13 and 14.

Compounds Y and Z of the comparative examples include both naphthobenzofuran structures and naphthobenzocarbazole substituents, but lack an arylamine group, leading to imbalanced charge transfer and charge balance, thereby resulting in performance degradation as seen in Comparative Examples 14 and 16.

Referring to Table 10, the organic light-emitting devices of the examples using one host had relatively low threshold voltages and operating voltages, efficiencies of 15.91 cd/A or more, and T90 lifetimes of 20 or more.

In contrast, the organic light-emitting devices of the comparative examples had relatively high threshold voltages and operating voltages, low efficiencies of 7.98 cd/A or less, and degraded T90 lifetimes of 15 or less.

Referring to Table 11, the organic light-emitting devices of the examples using two hosts had threshold voltages of 2.16 V or less, efficiencies of 66.07 cd/A or more, and T90 lifetimes of 105 or more.

In contrast, referring to Table 12, the organic light-emitting devices of the comparative examples had high threshold voltages of 2.3 V or more, low efficiencies of 63.52 cd/A or less, and degraded T90 lifetimes of 87 or less, demonstrating inferior performance compared to the organic light-emitting devices of the examples.

The contents described above are merely examples of applying the principles of the present disclosure, and other configurations may be further included without departing from the scope of the present disclosure.

What is claimed is:

1. A heterocyclic compound represented by Formula 1: 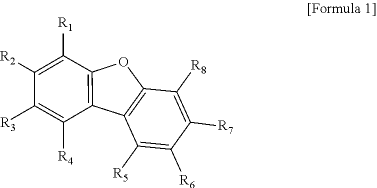
wherein two adjacent groups of R₁ to R₄ are linked to form an aromatic ring represented by 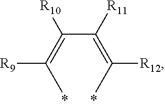
and
among the remaining two groups of R₁ to R₄ that do not form the aromatic ring and R₅ to R₁₂:
one is a group represented by Formula 2;
another is a benzocarbazole group having a substituted or unsubstituted aryl group having 6 to 60 carbon atoms or a substituted or unsubstituted heteroaryl group having 2 to 60 carbon atoms bonded to a nitrogen atom; and
the remaining ones are each independently selected from the groups consisting of hydrogen, deuterium, a halogen, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted alkoxy group having 1 to 60 carbon atoms, a substituted or unsubstituted cycloalkyl group having 3 to 60 carbon atoms, a substituted or unsubstituted heterocycloalkyl group having 2 to 60 carbon atoms, a substituted or unsubstituted aryl group having 6 to 60 carbon atoms, a substituted or unsubstituted heteroaryl group having 2 to 60 carbon atoms, —SiRR′R″, or —P(═O)RR′, wherein two or more adjacent groups among the remaining ones are optionally linked to form a substituted or unsubstituted 5- to 9-membered saturated or unsaturated ring; and
R, R′ and R″ are each independently hydrogen, deuterium, —CN, a substituted or unsubstituted alkyl group having 1 to 60 carbon atoms, a substituted or unsubstituted cycloalkyl group having 3 to 60 carbon atoms, a substituted or unsubstituted aryl group having 6 to 60 carbon atoms, or a substituted or unsubstituted heteroaryl group having 2 to 60 carbon atoms: 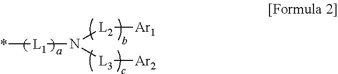
in Formula 2, Ar₁ and Ar₂ are each independently a substituted or unsubstituted aryl group having 6 to 60 carbon atoms or a substituted or unsubstituted heteroaryl group having 2 to 60 carbon atoms,
L₁, L₂, and L₃ are each independently a direct linkage, a substituted or unsubstituted arylene group having 6 to 60 carbon atoms, or a substituted or unsubstituted heteroarylene group having 2 to 60 carbon atoms,
a, b and c are each independently an integer of 0 to 5,
when a is an integer of 2 to 5, a plurality of L₁ groups are the same or different,
when b is an integer of 2 to 5, a plurality of L₂ groups are the same or different,
when c is an integer of 2 to 5, a plurality of L₃ groups are the same or different, and
* represents a bonding site.

2. The heterocyclic compound according to claim 1, wherein the heterocyclic compound is represented by any one selected from the groups consisting of Formulae 1-1 to 1-3: 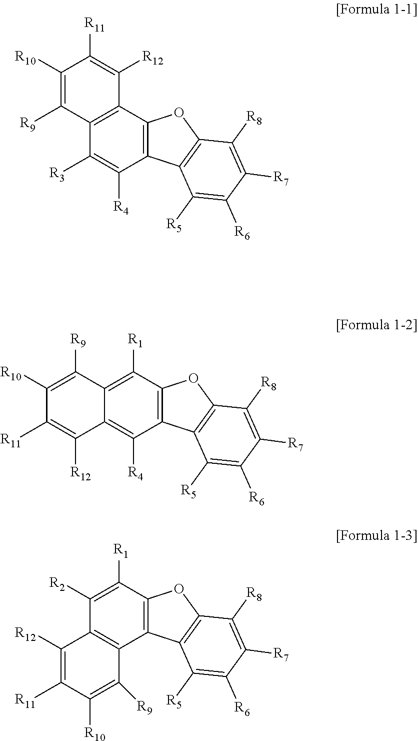
in Formulae 1-1 to 1-3, R₁ to R₁₂ are the same as defined in Formula 1.

3. The heterocyclic compound according to claim 2, wherein, in Formulae 1-1 to 1-3,
among the remaining two groups of R₁ to R₄ that do not form the ring and R₅ to R₈,
one is represented by Formula 2, another is a benzocarbazole group having a substituted or unsubstituted aryl group having 6 to 60 carbon atoms or a substituted or unsubstituted heteroaryl group having 2 to 60 carbon atoms bonded to a nitrogen atom, and
R₉ to R₁₂ are each independently hydrogen or deuterium.

4. The heterocyclic compound according to claim 1, wherein the benzocarbazole group is represented by any one selected from the groups consisting of Formulae 3-1 to 3-4: 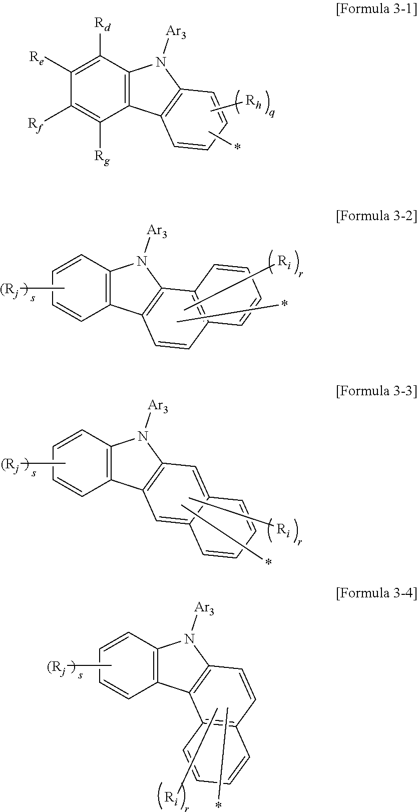
in Formulae 3-1 to 3-4, Ar₃ is an aryl group having 6 to 60 carbon atoms or a heteroaryl group having 2 to 60 carbon atoms,
two adjacent groups of R_{d}, Rₑ, R_{f} and R_{g} are linked to form an aromatic ring represented by 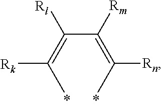
the remaining two groups of R_{d}, Rₑ, R_{f} and R_{g} that do not form the ring, and Rₕ, Rᵢ, Rⱼ, Rₖ, Rₗ, Rₘ and Rₙ are each independently hydrogen, deuterium, a halogen, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted alkoxy group having 1 to 60 carbon atoms, a substituted or unsubstituted cycloalkyl group having 3 to 60 carbon atoms, a substituted or unsubstituted heterocycloalkyl group having 2 to 60 carbon atoms, a substituted or unsubstituted aryl group having 6 to 60 carbon atoms, a substituted or unsubstituted heteroaryl group having 2 to 60 carbon atoms, —SiRR′R″, and —P(═O)RR′, wherein two or more adjacent groups among the remaining two groups of R_{d}, Rₑ, R_{f} and R_{g} that do not form the ring and Rₕ, Rᵢ, Rⱼ, Rₖ, Rₗ, Rₘ and Rₙ are optionally linked to form a substituted or unsubstituted 5- to 9-membered saturated or unsaturated ring, and
R, R′ and R″ are each independently hydrogen, deuterium, —CN, a substituted or unsubstituted alkyl group having 1 to 60 carbon atoms, a substituted or unsubstituted cycloalkyl group having 3 to 60 carbon atoms, a substituted or unsubstituted aryl group having 6 to 60 carbon atoms, or a substituted or unsubstituted heteroaryl group having 2 to 60 carbon atoms,
q is an integer of 0 to 3, and when q is 2 or 3, a plurality of Rₕ groups are the same or different,
r is an integer of 0 to 5, and when r is an integer from 2 to 5, a plurality of Rᵢ groups are the same or different, and
s is an integer of 0 to 4, and when s is an integer from 2 to 4, a plurality of Rⱼ groups are the same or different.

5. The heterocyclic compound according to claim 4, wherein, in Formulae 3-1 to 3-4, Ar₃ is a substituted or unsubstituted aryl group having 6 to 15 carbon atoms.

6. The heterocyclic compound according to claim 1, wherein, in Formula 2, L₁ is a direct linkage or a substituted or unsubstituted arylene group having 6 to 15 carbon atoms.

7. The heterocyclic compound according to claim 1, wherein the deuterium content of the heterocyclic compound is 0, or greater than 0 and not more than 100%.

8. The heterocyclic compound according to claim 1, wherein the heterocyclic compound is represented by any one of Formulae below:

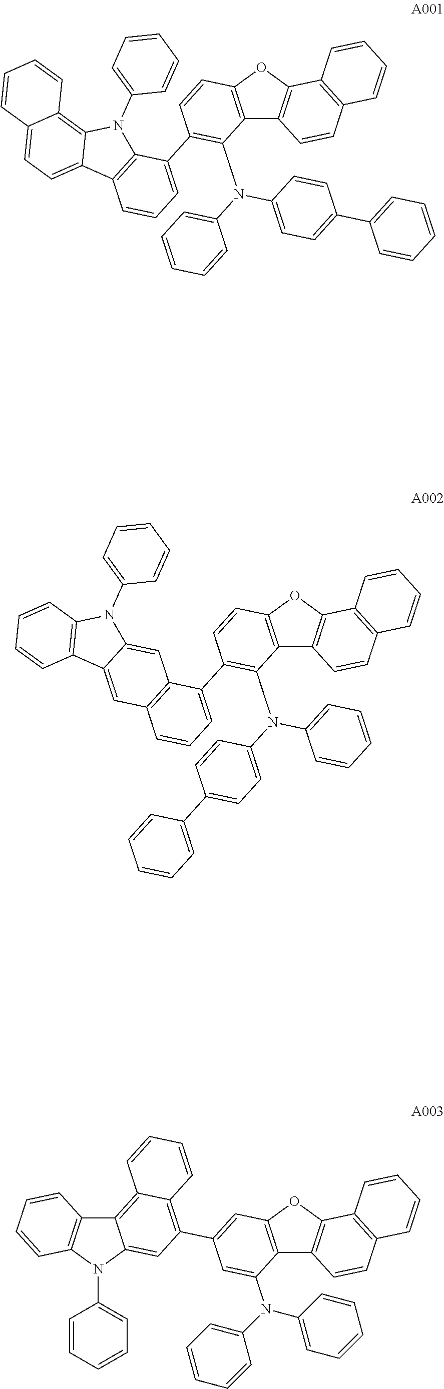 [SMILES: c1ccc(-c2ccc(N(c3ccccc3)c3c(-c4cccc5c6ccc7ccccc7c6n(-c6ccccc6)c45)ccc4oc5c6ccccc6ccc5c34)cc2)cc1 ; c1ccc(-c2ccc(N(c3ccccc3)c3c(-c4cccc5cc6c7ccccc7n(-c7ccccc7)c6cc45)ccc4oc5c6ccccc6ccc5c34)cc2)cc1 ; c1ccc(N(c2ccccc2)c2cc(-c3cc4c(c5ccccc35)c3ccccc3n4-c3ccccc3)cc3oc4c5ccccc5ccc4c23)cc1]

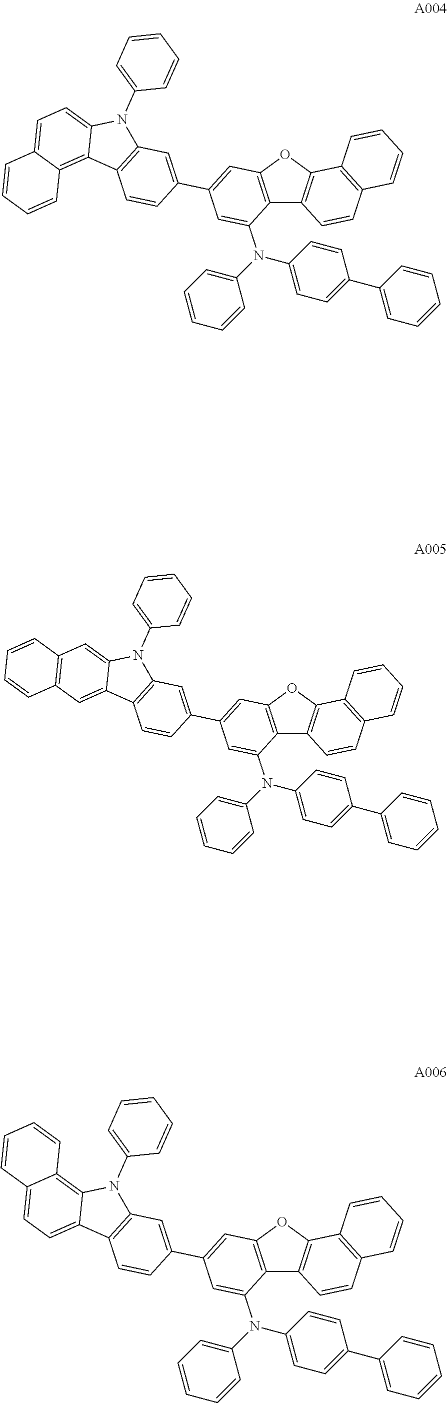 [SMILES: c1ccc(-c2ccc(N(c3ccccc3)c3cc(-c4ccc5c6c7ccccc7ccc6n(-c6ccccc6)c5c4)cc4oc5c6ccccc6ccc5c34)cc2)cc1 ; c1ccc(-c2ccc(N(c3ccccc3)c3cc(-c4ccc5c6cc7ccccc7cc6n(-c6ccccc6)c5c4)cc4oc5c6ccccc6ccc5c34)cc2)cc1 ; c1ccc(-c2ccc(N(c3ccccc3)c3cc(-c4ccc5c6ccc7ccccc7c6n(-c6ccccc6)c5c4)cc4oc5c6ccccc6ccc5c34)cc2)cc1]

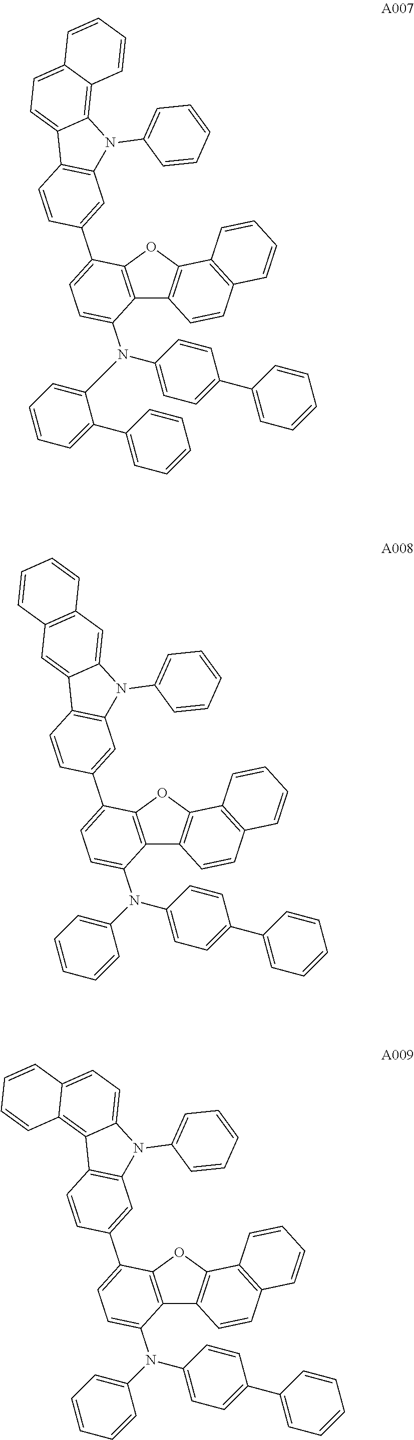 [SMILES: c1ccc(-c2ccc(N(c3ccccc3-c3ccccc3)c3ccc(-c4ccc5c6ccc7ccccc7c6n(-c6ccccc6)c5c4)c4oc5c6ccccc6ccc5c34)cc2)cc1 ; c1ccc(-c2ccc(N(c3ccccc3)c3ccc(-c4ccc5c6cc7ccccc7cc6n(-c6ccccc6)c5c4)c4oc5c6ccccc6ccc5c34)cc2)cc1 ; c1ccc(-c2ccc(N(c3ccccc3)c3ccc(-c4ccc5c6c7ccccc7ccc6n(-c6ccccc6)c5c4)c4oc5c6ccccc6ccc5c34)cc2)cc1]

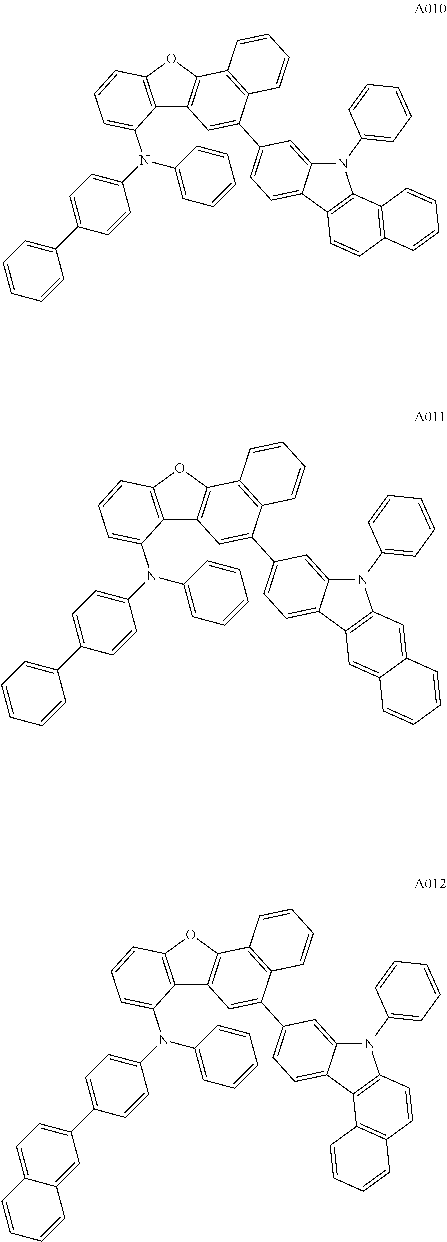 [SMILES: c1ccc(-c2ccc(N(c3ccccc3)c3cccc4oc5c6ccccc6c(-c6ccc7c8ccc9ccccc9c8n(-c8ccccc8)c7c6)cc5c34)cc2)cc1 ; c1ccc(-c2ccc(N(c3ccccc3)c3cccc4oc5c6ccccc6c(-c6ccc7c8cc9ccccc9cc8n(-c8ccccc8)c7c6)cc5c34)cc2)cc1 ; c1ccc(N(c2ccc(-c3ccc4ccccc4c3)cc2)c2cccc3oc4c5ccccc5c(-c5ccc6c7c8ccccc8ccc7n(-c7ccccc7)c6c5)cc4c23)cc1]

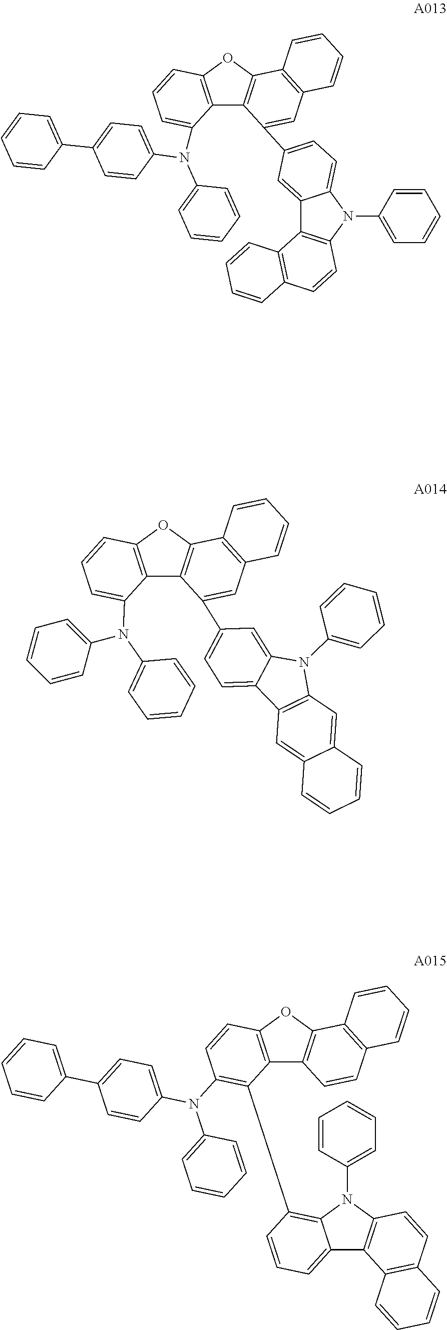 [SMILES: c1ccc(-c2ccc(N(c3ccccc3)c3cccc4oc5c6ccccc6cc(-c6ccc7c(c6)c6c8ccccc8ccc6n7-c6ccccc6)c5c34)cc2)cc1 ; c1ccc(N(c2ccccc2)c2cccc3oc4c5ccccc5cc(-c5ccc6c7cc8ccccc8cc7n(-c7ccccc7)c6c5)c4c23)cc1 ; c1ccc(-c2ccc(N(c3ccccc3)c3ccc4oc5c6ccccc6ccc5c4c3-c3cccc4c5c6ccccc6ccc5n(-c5ccccc5)c34)cc2)cc1]

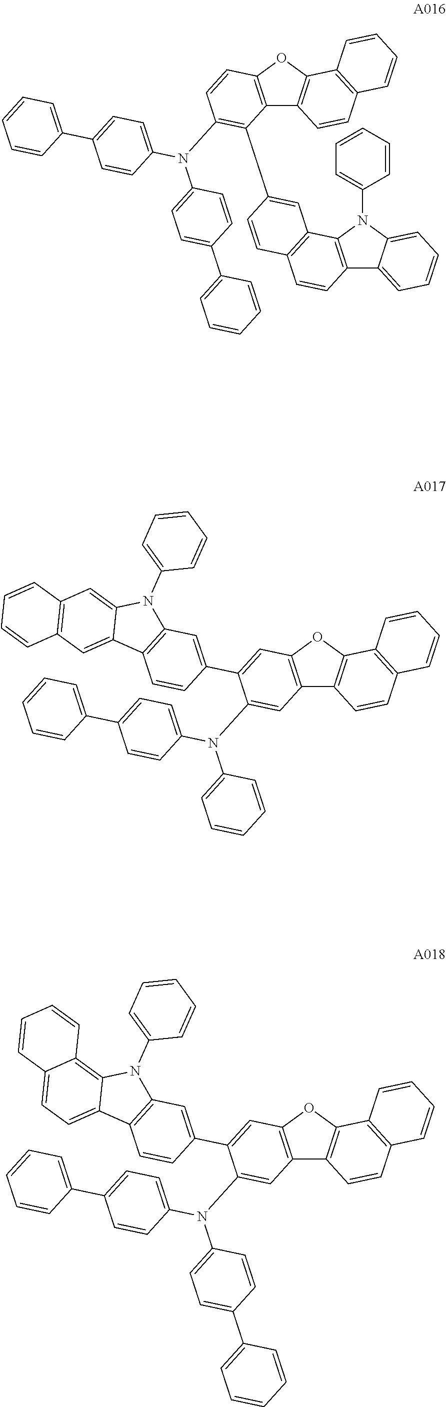 [SMILES: c1ccc(-c2ccc(N(c3ccc(-c4ccccc4)cc3)c3ccc4oc5c6ccccc6ccc5c4c3-c3ccc4ccc5c6ccccc6n(-c6ccccc6)c5c4c3)cc2)cc1 ; c1ccc(-c2ccc(N(c3ccccc3)c3cc4c(cc3-c3ccc5c6cc7ccccc7cc6n(-c6ccccc6)c5c3)oc3c5ccccc5ccc43)cc2)cc1 ; c1ccc(-c2ccc(N(c3ccc(-c4ccccc4)cc3)c3cc4c(cc3-c3ccc5c6ccc7ccccc7c6n(-c6ccccc6)c5c3)oc3c5ccccc5ccc43)cc2)cc1]

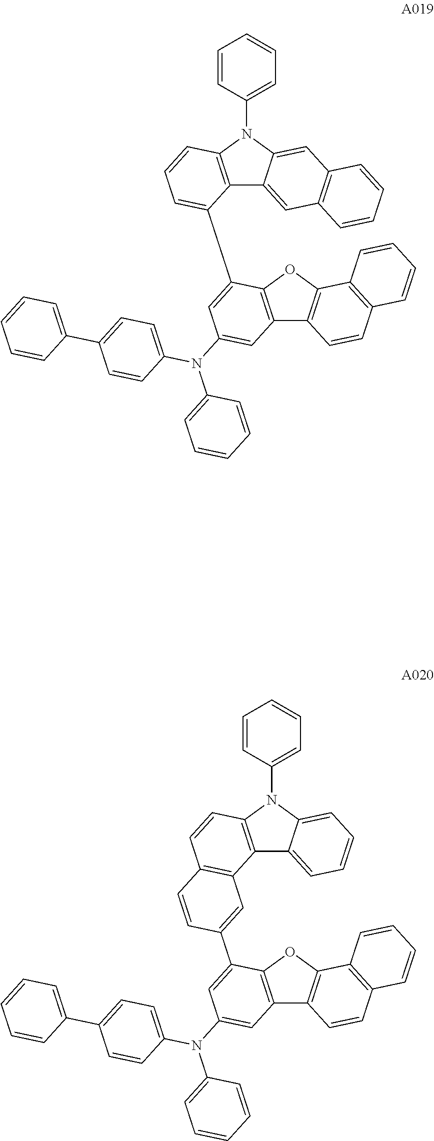 [SMILES: c1ccc(-c2ccc(N(c3ccccc3)c3cc(-c4cccc5c4c4cc6ccccc6cc4n5-c4ccccc4)c4oc5c6ccccc6ccc5c4c3)cc2)cc1 ; c1ccc(-c2ccc(N(c3ccccc3)c3cc(-c4ccc5ccc6c(c5c4)c4ccccc4n6-c4ccccc4)c4oc5c6ccccc6ccc5c4c3)cc2)cc1]

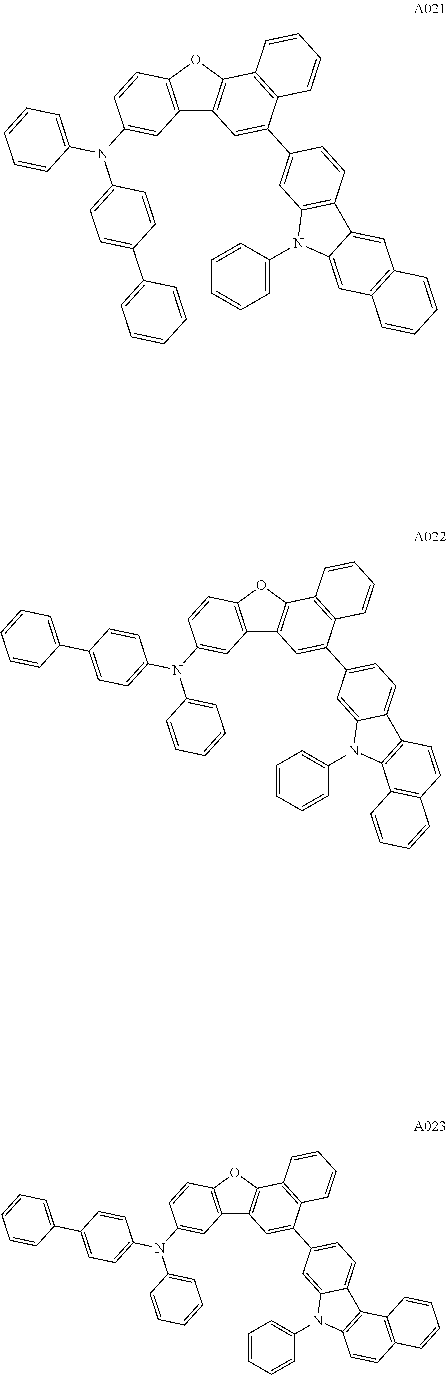 [SMILES: c1ccc(-c2ccc(N(c3ccccc3)c3ccc4oc5c6ccccc6c(-c6ccc7c8cc9ccccc9cc8n(-c8ccccc8)c7c6)cc5c4c3)cc2)cc1 ; c1ccc(-c2ccc(N(c3ccccc3)c3ccc4oc5c6ccccc6c(-c6ccc7c8ccc9ccccc9c8n(-c8ccccc8)c7c6)cc5c4c3)cc2)cc1 ; c1ccc(-c2ccc(N(c3ccccc3)c3ccc4oc5c6ccccc6c(-c6ccc7c8c9ccccc9ccc8n(-c8ccccc8)c7c6)cc5c4c3)cc2)cc1]

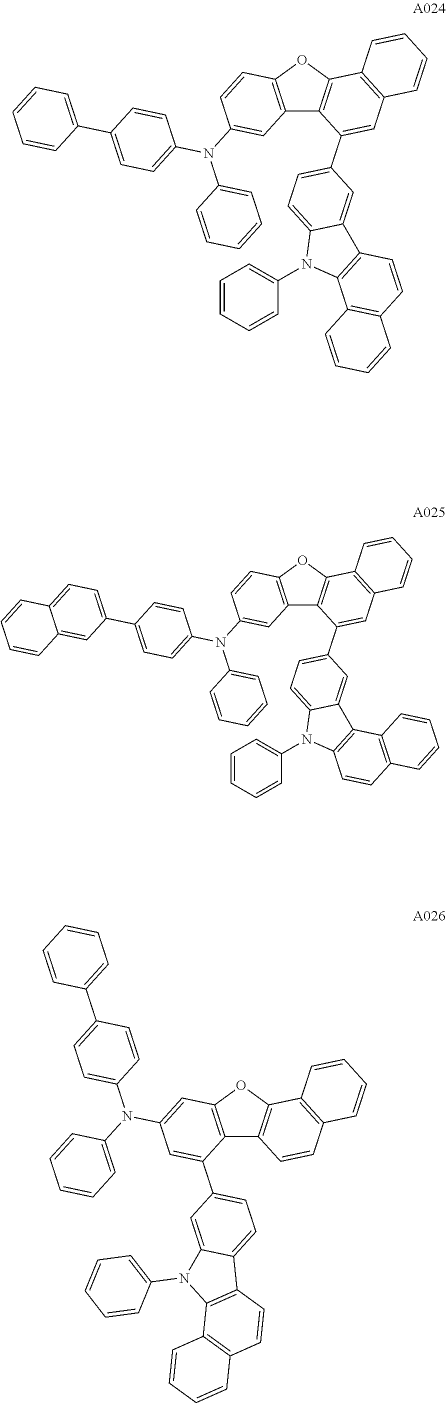 [SMILES: c1ccc(-c2ccc(N(c3ccccc3)c3ccc4oc5c6ccccc6cc(-c6ccc7c(c6)c6ccc8ccccc8c6n7-c6ccccc6)c5c4c3)cc2)cc1 ; c1ccc(N(c2ccc(-c3ccc4ccccc4c3)cc2)c2ccc3oc4c5ccccc5cc(-c5ccc6c(c5)c5c7ccccc7ccc5n6-c5ccccc5)c4c3c2)cc1 ; c1ccc(-c2ccc(N(c3ccccc3)c3cc(-c4ccc5c6ccc7ccccc7c6n(-c6ccccc6)c5c4)c4c(c3)oc3c5ccccc5ccc34)cc2)cc1]

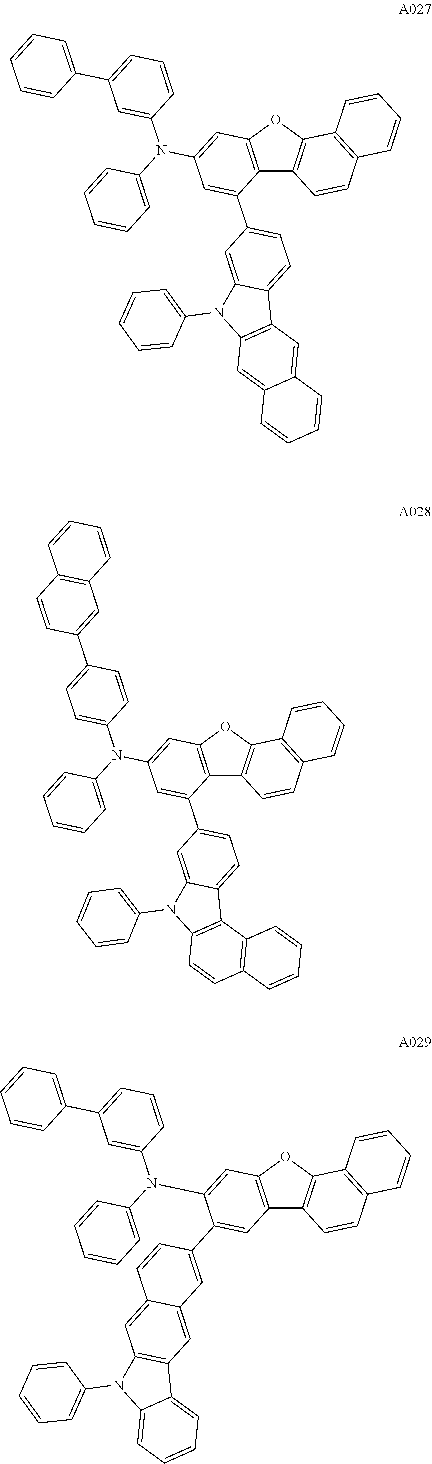 [SMILES: c1ccc(-c2cccc(N(c3ccccc3)c3cc(-c4ccc5c6cc7ccccc7cc6n(-c6ccccc6)c5c4)c4c(c3)oc3c5ccccc5ccc34)c2)cc1 ; c1ccc(N(c2ccc(-c3ccc4ccccc4c3)cc2)c2cc(-c3ccc4c5c6ccccc6ccc5n(-c5ccccc5)c4c3)c3c(c2)oc2c4ccccc4ccc23)cc1 ; c1ccc(-c2cccc(N(c3ccccc3)c3cc4oc5c6ccccc6ccc5c4cc3-c3ccc4cc5c(cc4c3)c3ccccc3n5-c3ccccc3)c2)cc1]

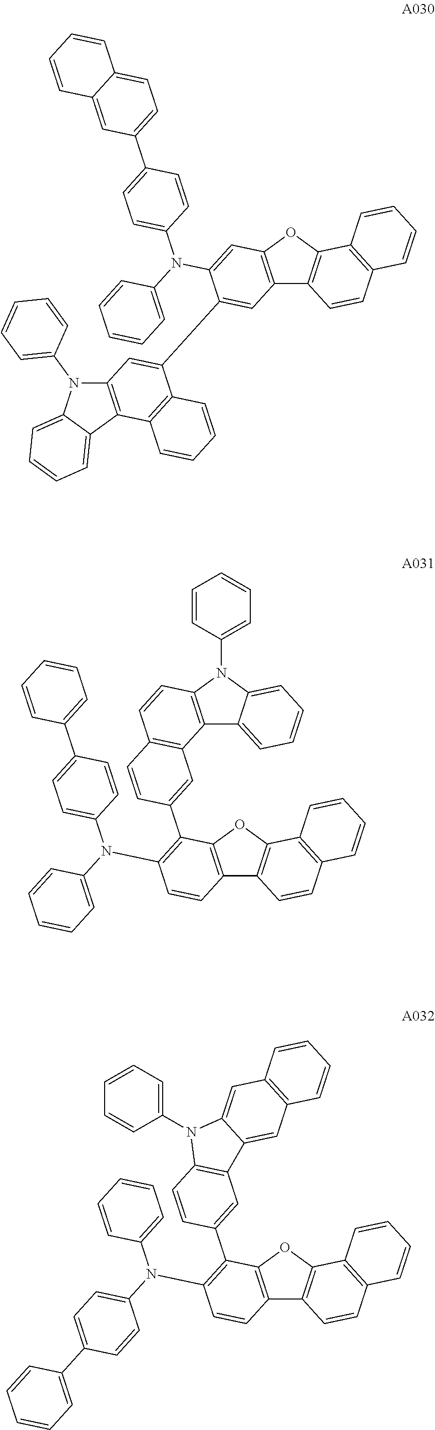 [SMILES: c1ccc(N(c2ccc(-c3ccc4ccccc4c3)cc2)c2cc3oc4c5ccccc5ccc4c3cc2-c2cc3c(c4ccccc24)c2ccccc2n3-c2ccccc2)cc1 ; c1ccc(-c2ccc(N(c3ccccc3)c3ccc4c(oc5c6ccccc6ccc45)c3-c3ccc4ccc5c(c4c3)c3ccccc3n5-c3ccccc3)cc2)cc1 ; c1ccc(-c2ccc(N(c3ccccc3)c3ccc4c(oc5c6ccccc6ccc45)c3-c3ccc4c(c3)c3cc5ccccc5cc3n4-c3ccccc3)cc2)cc1]

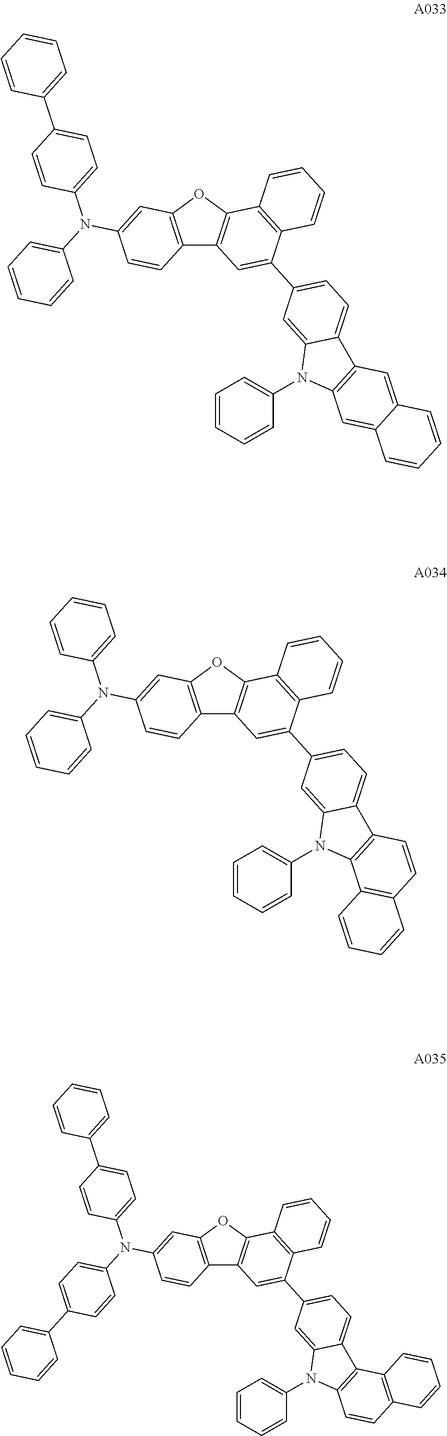 [SMILES: c1ccc(-c2ccc(N(c3ccccc3)c3ccc4c(c3)oc3c5ccccc5c(-c5ccc6c7cc8ccccc8cc7n(-c7ccccc7)c6c5)cc43)cc2)cc1 ; c1ccc(N(c2ccccc2)c2ccc3c(c2)oc2c4ccccc4c(-c4ccc5c6ccc7ccccc7c6n(-c6ccccc6)c5c4)cc32)cc1 ; c1ccc(-c2ccc(N(c3ccc(-c4ccccc4)cc3)c3ccc4c(c3)oc3c5ccccc5c(-c5ccc6c7c8ccccc8ccc7n(-c7ccccc7)c6c5)cc43)cc2)cc1]

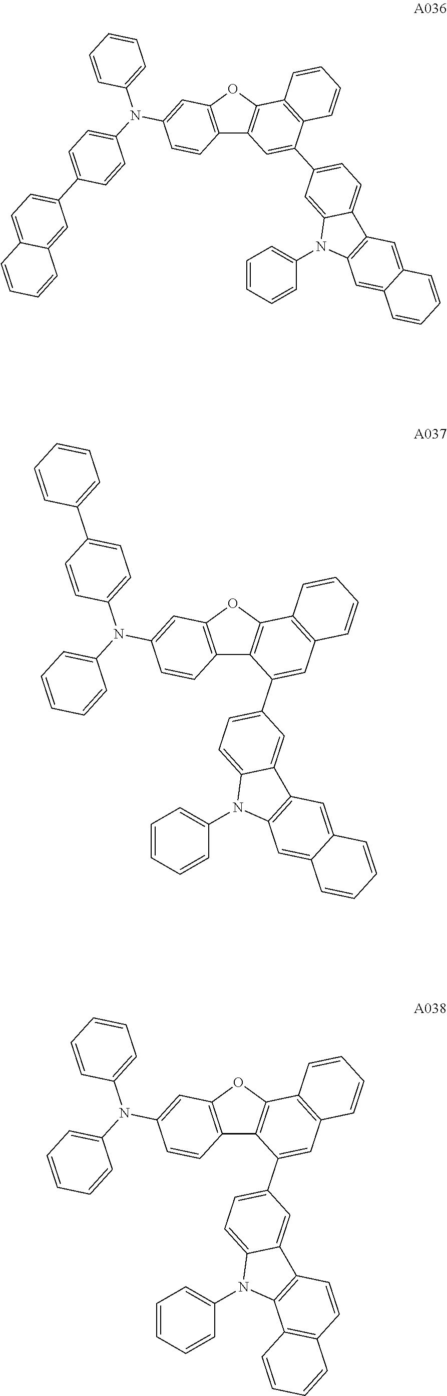 [SMILES: c1ccc(N(c2ccc(-c3ccc4ccccc4c3)cc2)c2ccc3c(c2)oc2c4ccccc4c(-c4ccc5c6cc7ccccc7cc6n(-c6ccccc6)c5c4)cc32)cc1 ; c1ccc(-c2ccc(N(c3ccccc3)c3ccc4c(c3)oc3c5ccccc5cc(-c5ccc6c(c5)c5cc7ccccc7cc5n6-c5ccccc5)c43)cc2)cc1 ; c1ccc(N(c2ccccc2)c2ccc3c(c2)oc2c4ccccc4cc(-c4ccc5c(c4)c4ccc6ccccc6c4n5-c4ccccc4)c32)cc1]

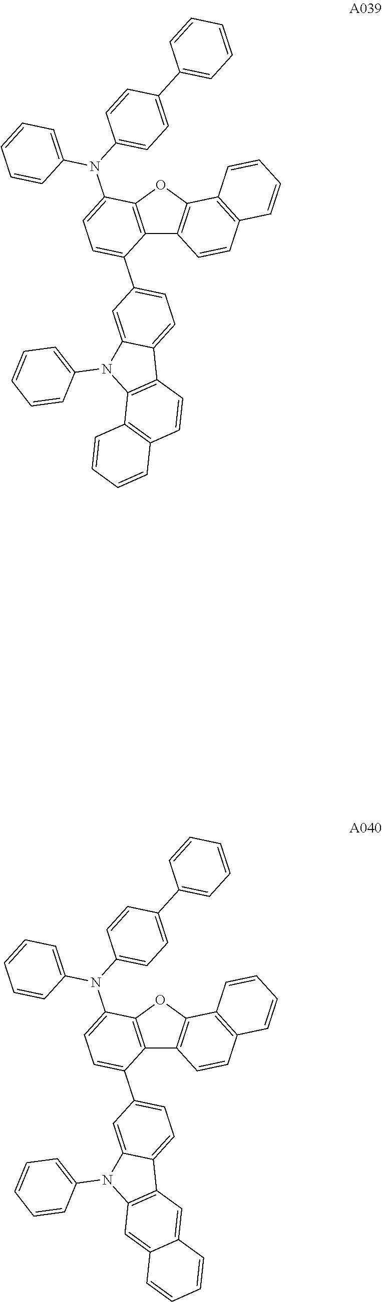 [SMILES: c1ccc(-c2ccc(N(c3ccccc3)c3ccc(-c4ccc5c6ccc7ccccc7c6n(-c6ccccc6)c5c4)c4c3oc3c5ccccc5ccc34)cc2)cc1 ; c1ccc(-c2ccc(N(c3ccccc3)c3ccc(-c4ccc5c6cc7ccccc7cc6n(-c6ccccc6)c5c4)c4c3oc3c5ccccc5ccc34)cc2)cc1]

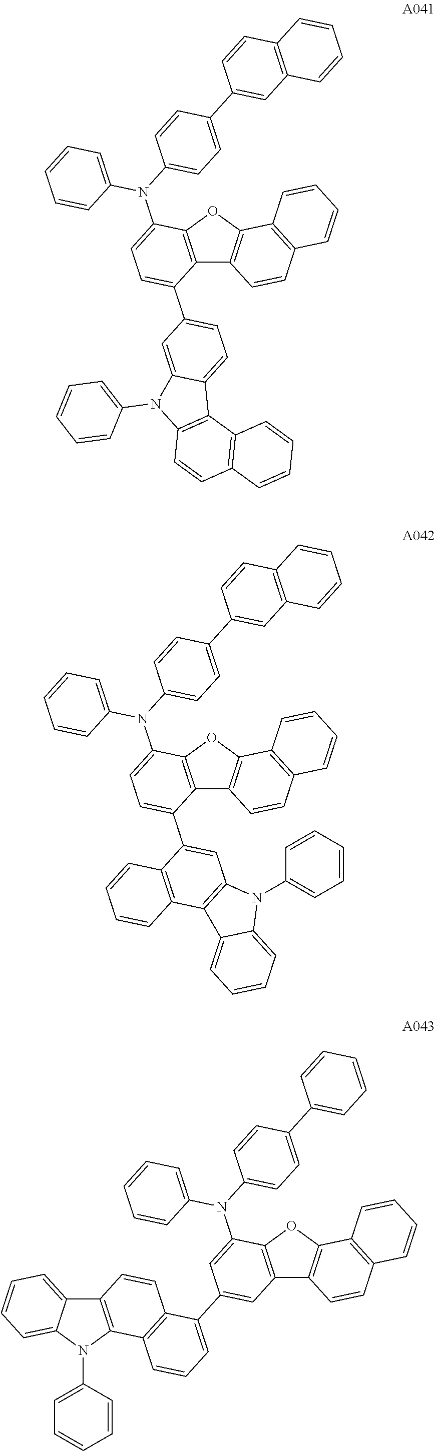 [SMILES: c1ccc(N(c2ccc(-c3ccc4ccccc4c3)cc2)c2ccc(-c3ccc4c5c6ccccc6ccc5n(-c5ccccc5)c4c3)c3c2oc2c4ccccc4ccc23)cc1 ; c1ccc(N(c2ccc(-c3ccc4ccccc4c3)cc2)c2ccc(-c3cc4c(c5ccccc35)c3ccccc3n4-c3ccccc3)c3c2oc2c4ccccc4ccc23)cc1 ; c1ccc(-c2ccc(N(c3ccccc3)c3cc(-c4cccc5c4ccc4c6ccccc6n(-c6ccccc6)c54)cc4c3oc3c5ccccc5ccc43)cc2)cc1]

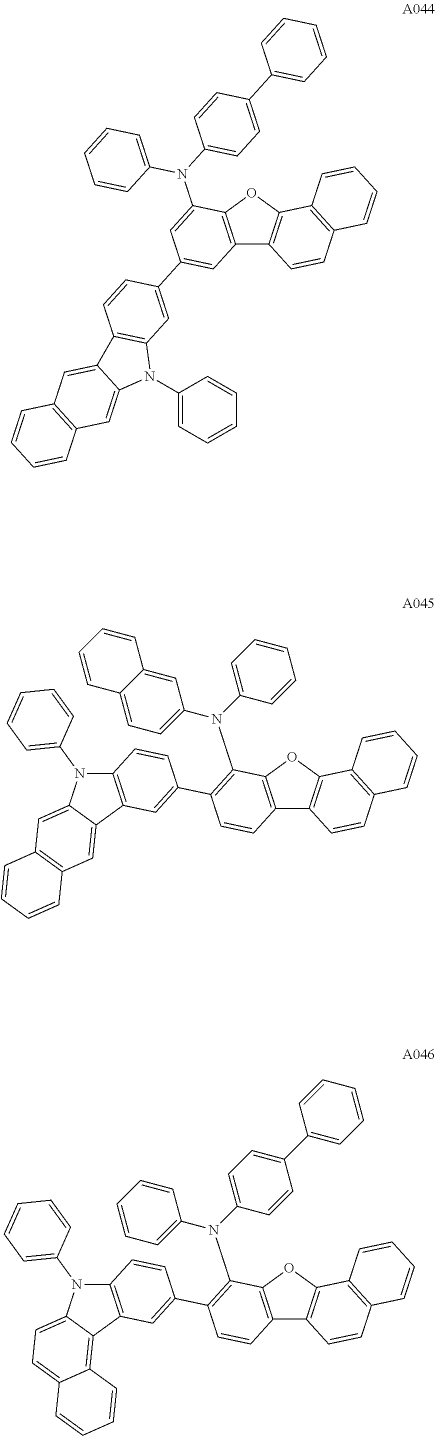 [SMILES: c1ccc(-c2ccc(N(c3ccccc3)c3cc(-c4ccc5c6cc7ccccc7cc6n(-c6ccccc6)c5c4)cc4c3oc3c5ccccc5ccc43)cc2)cc1 ; c1ccc(N(c2ccc3ccccc3c2)c2c(-c3ccc4c(c3)c3cc5ccccc5cc3n4-c3ccccc3)ccc3c2oc2c4ccccc4ccc32)cc1 ; c1ccc(-c2ccc(N(c3ccccc3)c3c(-c4ccc5c(c4)c4c6ccccc6ccc4n5-c4ccccc4)ccc4c3oc3c5ccccc5ccc43)cc2)cc1]

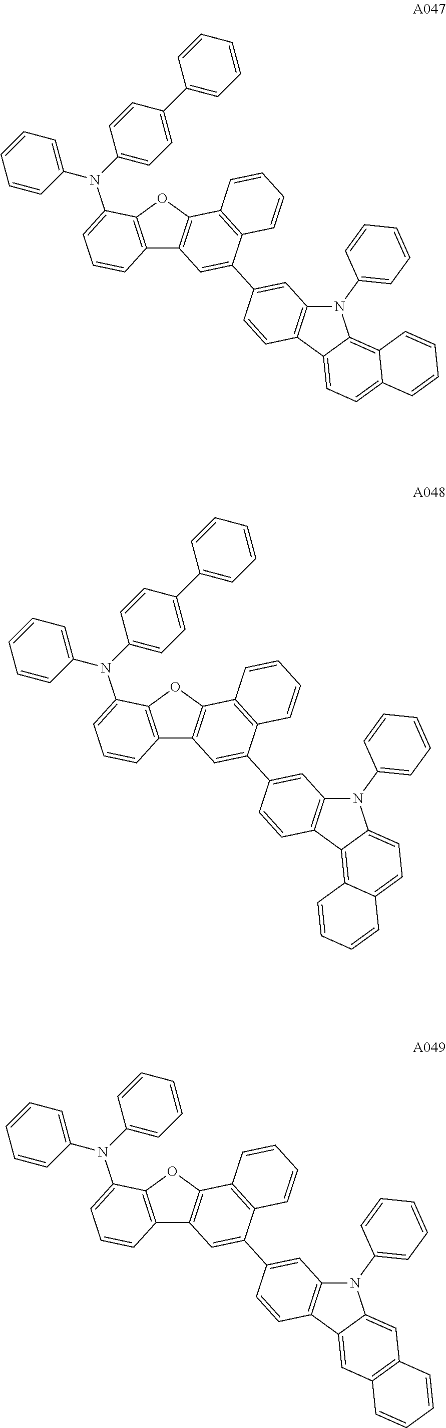 [SMILES: c1ccc(-c2ccc(N(c3ccccc3)c3cccc4c3oc3c5ccccc5c(-c5ccc6c7ccc8ccccc8c7n(-c7ccccc7)c6c5)cc43)cc2)cc1 ; c1ccc(-c2ccc(N(c3ccccc3)c3cccc4c3oc3c5ccccc5c(-c5ccc6c7c8ccccc8ccc7n(-c7ccccc7)c6c5)cc43)cc2)cc1 ; c1ccc(N(c2ccccc2)c2cccc3c2oc2c4ccccc4c(-c4ccc5c6cc7ccccc7cc6n(-c6ccccc6)c5c4)cc32)cc1]

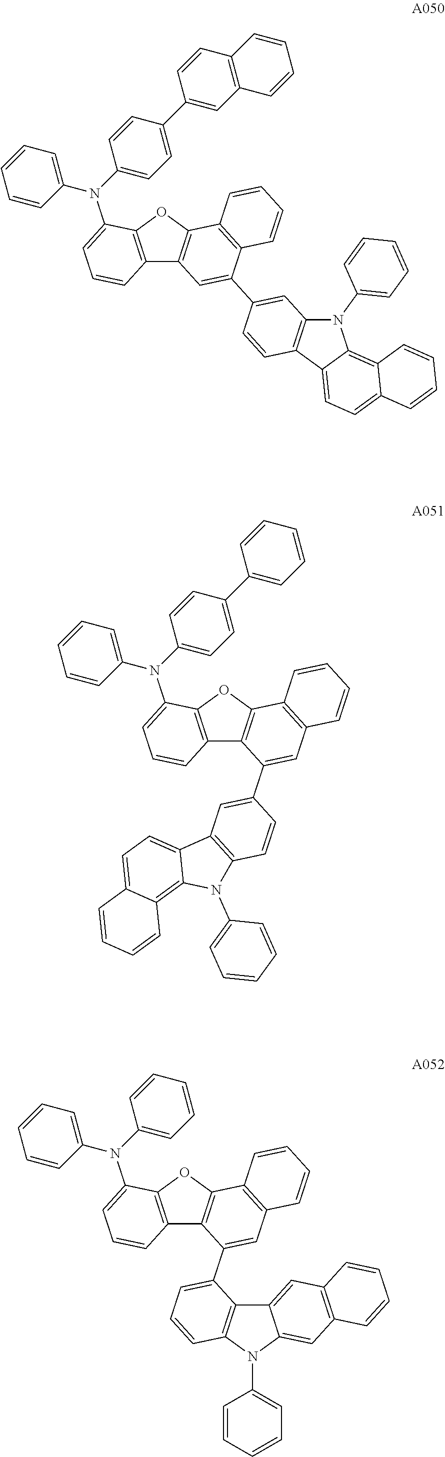 [SMILES: c1ccc(N(c2ccc(-c3ccc4ccccc4c3)cc2)c2cccc3c2oc2c4ccccc4c(-c4ccc5c6ccc7ccccc7c6n(-c6ccccc6)c5c4)cc32)cc1 ; c1ccc(-c2ccc(N(c3ccccc3)c3cccc4c3oc3c5ccccc5cc(-c5ccc6c(c5)c5ccc7ccccc7c5n6-c5ccccc5)c43)cc2)cc1 ; c1ccc(N(c2ccccc2)c2cccc3c2oc2c4ccccc4cc(-c4cccc5c4c4cc6ccccc6cc4n5-c4ccccc4)c32)cc1]

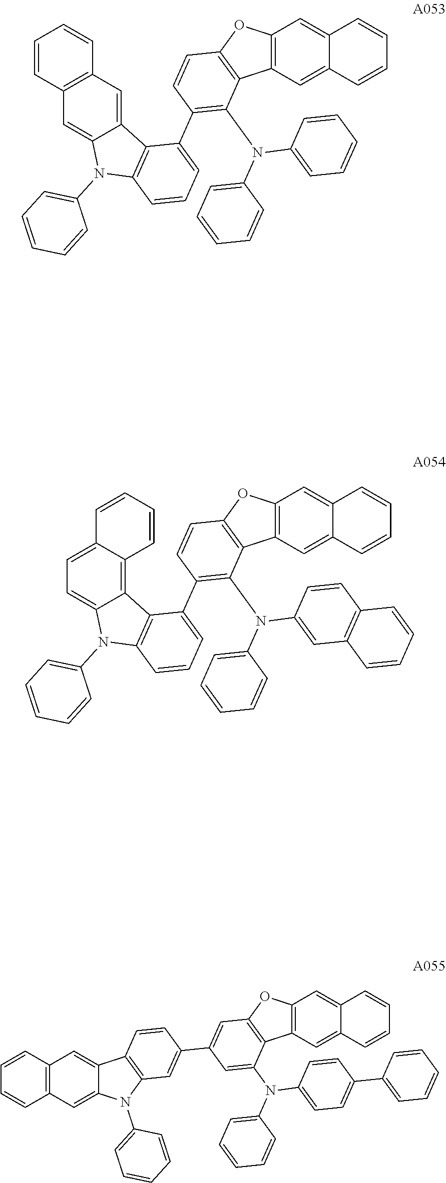 [SMILES: c1ccc(N(c2ccccc2)c2c(-c3cccc4c3c3cc5ccccc5cc3n4-c3ccccc3)ccc3oc4cc5ccccc5cc4c23)cc1 ; c1ccc(N(c2ccc3ccccc3c2)c2c(-c3cccc4c3c3c5ccccc5ccc3n4-c3ccccc3)ccc3oc4cc5ccccc5cc4c23)cc1 ; c1ccc(-c2ccc(N(c3ccccc3)c3cc(-c4ccc5c6cc7ccccc7cc6n(-c6ccccc6)c5c4)cc4oc5cc6ccccc6cc5c34)cc2)cc1]

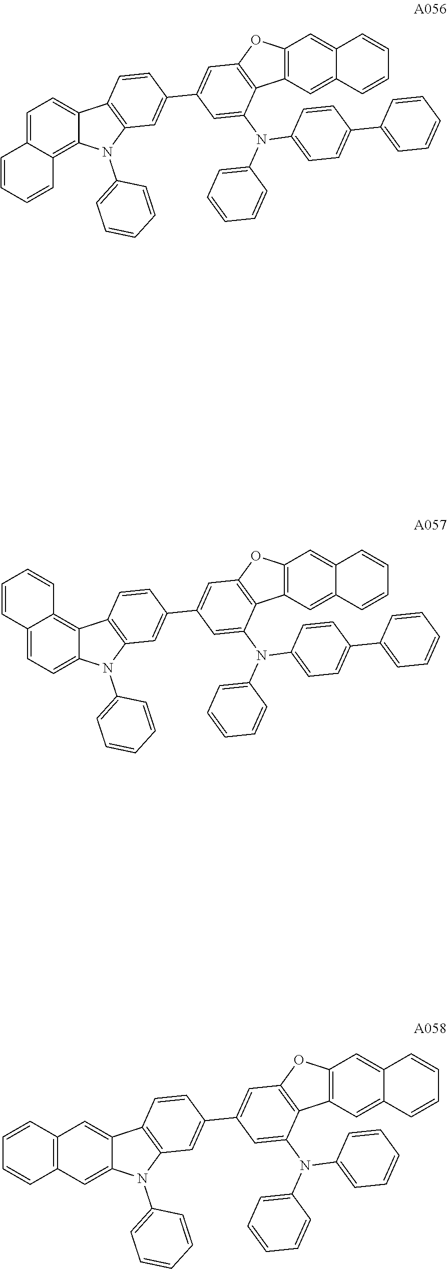 [SMILES: c1ccc(-c2ccc(N(c3ccccc3)c3cc(-c4ccc5c6ccc7ccccc7c6n(-c6ccccc6)c5c4)cc4oc5cc6ccccc6cc5c34)cc2)cc1 ; c1ccc(-c2ccc(N(c3ccccc3)c3cc(-c4ccc5c6c7ccccc7ccc6n(-c6ccccc6)c5c4)cc4oc5cc6ccccc6cc5c34)cc2)cc1 ; c1ccc(N(c2ccccc2)c2cc(-c3ccc4c5cc6ccccc6cc5n(-c5ccccc5)c4c3)cc3oc4cc5ccccc5cc4c23)cc1]

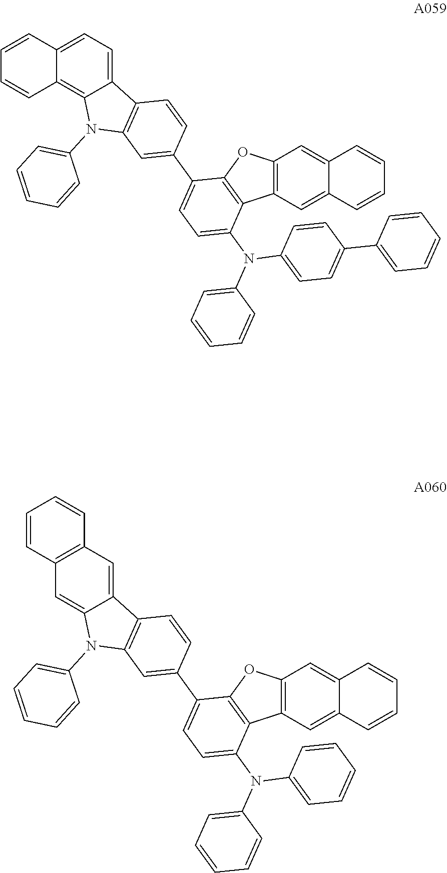 [SMILES: c1ccc(-c2ccc(N(c3ccccc3)c3ccc(-c4ccc5c6ccc7ccccc7c6n(-c6ccccc6)c5c4)c4oc5cc6ccccc6cc5c34)cc2)cc1 ; c1ccc(N(c2ccccc2)c2ccc(-c3ccc4c5cc6ccccc6cc5n(-c5ccccc5)c4c3)c3oc4cc5ccccc5cc4c23)cc1]

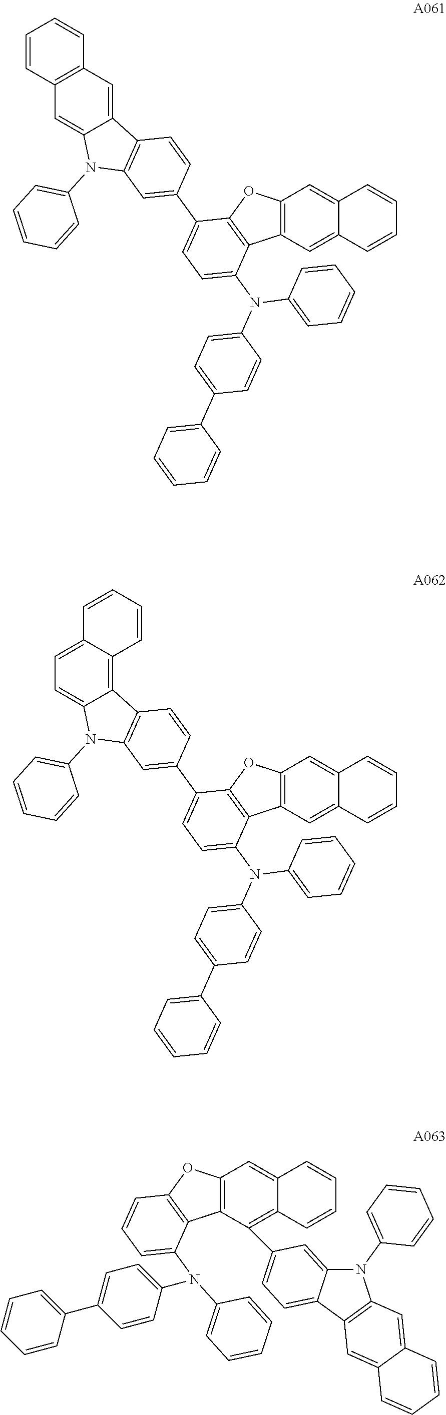 [SMILES: c1ccc(-c2ccc(N(c3ccccc3)c3ccc(-c4ccc5c6cc7ccccc7cc6n(-c6ccccc6)c5c4)c4oc5cc6ccccc6cc5c34)cc2)cc1 ; c1ccc(-c2ccc(N(c3ccccc3)c3ccc(-c4ccc5c6c7ccccc7ccc6n(-c6ccccc6)c5c4)c4oc5cc6ccccc6cc5c34)cc2)cc1 ; c1ccc(-c2ccc(N(c3ccccc3)c3cccc4oc5cc6ccccc6c(-c6ccc7c8cc9ccccc9cc8n(-c8ccccc8)c7c6)c5c34)cc2)cc1]

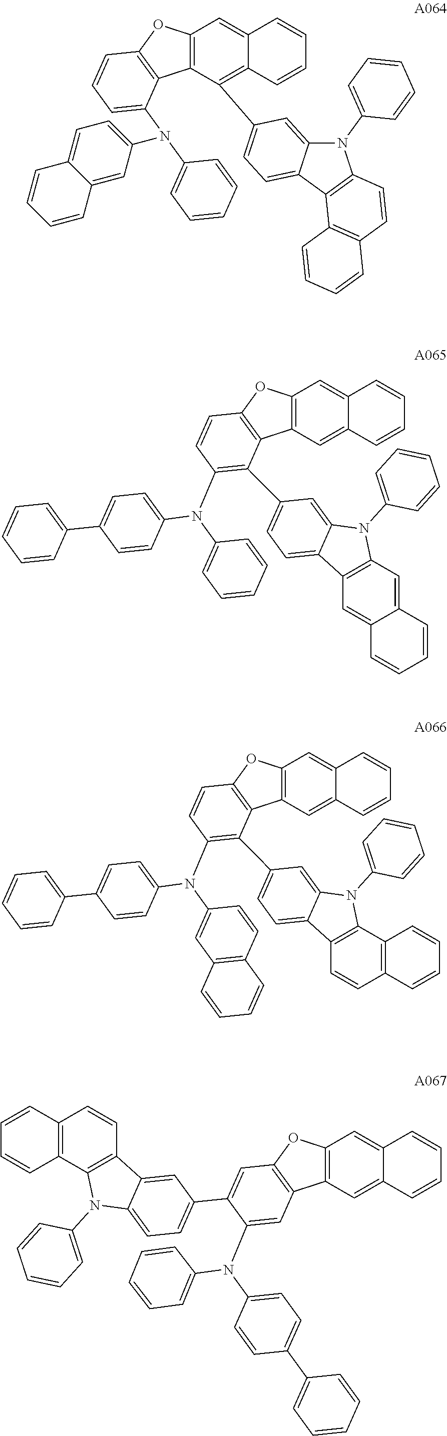 [SMILES: c1ccc(N(c2ccc3ccccc3c2)c2cccc3oc4cc5ccccc5c(-c5ccc6c7c8ccccc8ccc7n(-c7ccccc7)c6c5)c4c23)cc1 ; c1ccc(-c2ccc(N(c3ccccc3)c3ccc4oc5cc6ccccc6cc5c4c3-c3ccc4c5cc6ccccc6cc5n(-c5ccccc5)c4c3)cc2)cc1 ; c1ccc(-c2ccc(N(c3ccc4ccccc4c3)c3ccc4oc5cc6ccccc6cc5c4c3-c3ccc4c5ccc6ccccc6c5n(-c5ccccc5)c4c3)cc2)cc1 ; c1ccc(-c2ccc(N(c3ccccc3)c3cc4c(cc3-c3ccc5c(c3)c3ccc6ccccc6c3n5-c3ccccc3)oc3cc5ccccc5cc34)cc2)cc1]

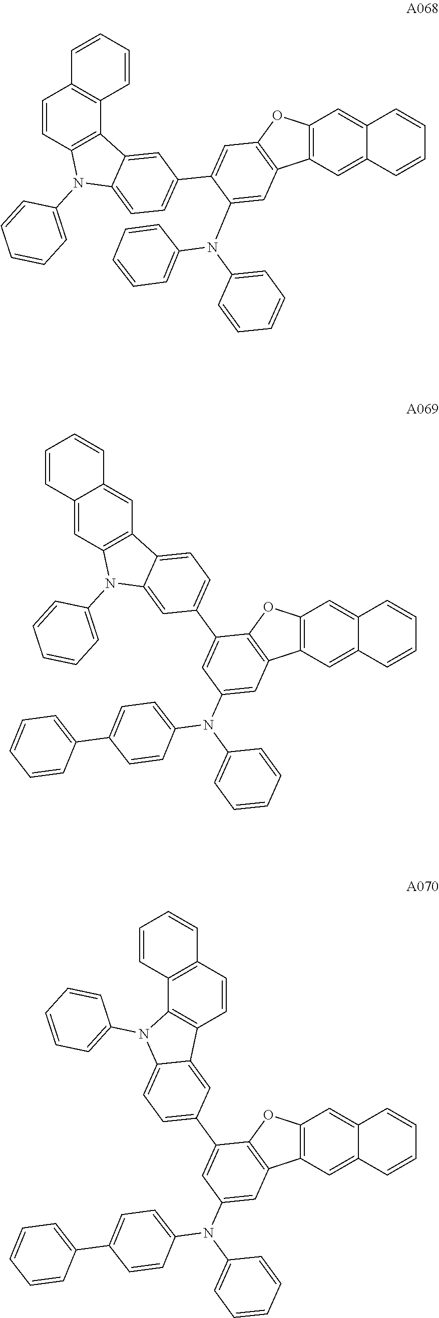 [SMILES: c1ccc(N(c2ccccc2)c2cc3c(cc2-c2ccc4c(c2)c2c5ccccc5ccc2n4-c2ccccc2)oc2cc4ccccc4cc23)cc1 ; c1ccc(-c2ccc(N(c3ccccc3)c3cc(-c4ccc5c6cc7ccccc7cc6n(-c6ccccc6)c5c4)c4oc5cc6ccccc6cc5c4c3)cc2)cc1 ; c1ccc(-c2ccc(N(c3ccccc3)c3cc(-c4ccc5c(c4)c4ccc6ccccc6c4n5-c4ccccc4)c4oc5cc6ccccc6cc5c4c3)cc2)cc1]

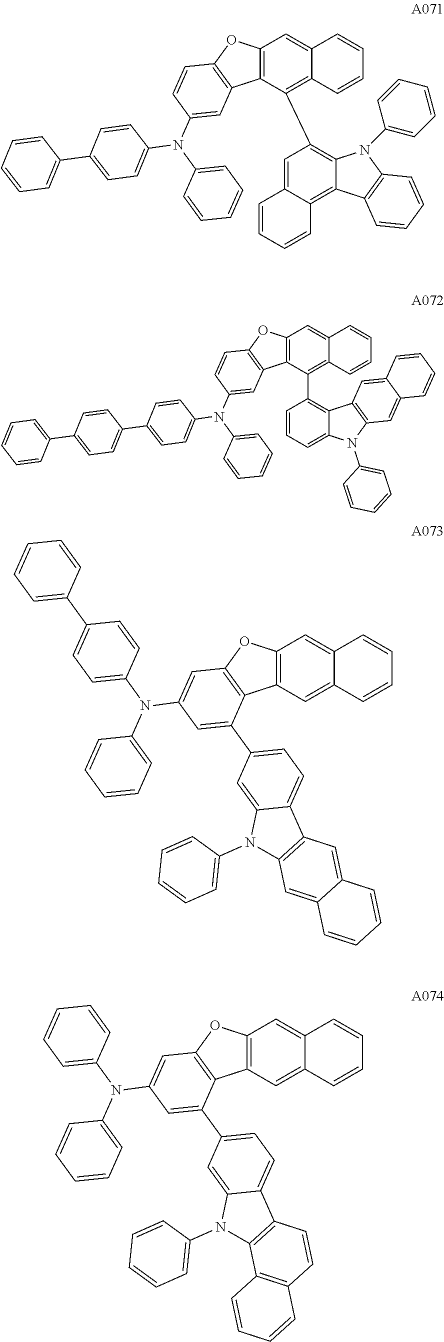 [SMILES: c1ccc(-c2ccc(N(c3ccccc3)c3ccc4oc5cc6ccccc6c(-c6cc7ccccc7c7c8ccccc8n(-c8ccccc8)c67)c5c4c3)cc2)cc1 ; c1ccc(-c2ccc(-c3ccc(N(c4ccccc4)c4ccc5oc6cc7ccccc7c(-c7cccc8c7c7cc9ccccc9cc7n8-c7ccccc7)c6c5c4)cc3)cc2)cc1 ; c1ccc(-c2ccc(N(c3ccccc3)c3cc(-c4ccc5c6cc7ccccc7cc6n(-c6ccccc6)c5c4)c4c(c3)oc3cc5ccccc5cc34)cc2)cc1 ; c1ccc(N(c2ccccc2)c2cc(-c3ccc4c5ccc6ccccc6c5n(-c5ccccc5)c4c3)c3c(c2)oc2cc4ccccc4cc23)cc1]

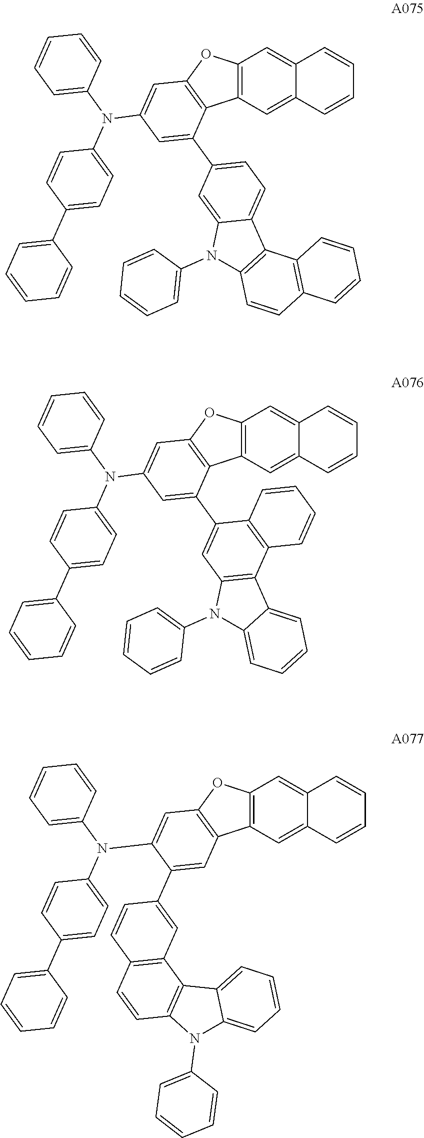 [SMILES: c1ccc(-c2ccc(N(c3ccccc3)c3cc(-c4ccc5c6c7ccccc7ccc6n(-c6ccccc6)c5c4)c4c(c3)oc3cc5ccccc5cc34)cc2)cc1 ; c1ccc(-c2ccc(N(c3ccccc3)c3cc(-c4cc5c(c6ccccc46)c4ccccc4n5-c4ccccc4)c4c(c3)oc3cc5ccccc5cc34)cc2)cc1 ; c1ccc(-c2ccc(N(c3ccccc3)c3cc4oc5cc6ccccc6cc5c4cc3-c3ccc4ccc5c(c4c3)c3ccccc3n5-c3ccccc3)cc2)cc1]

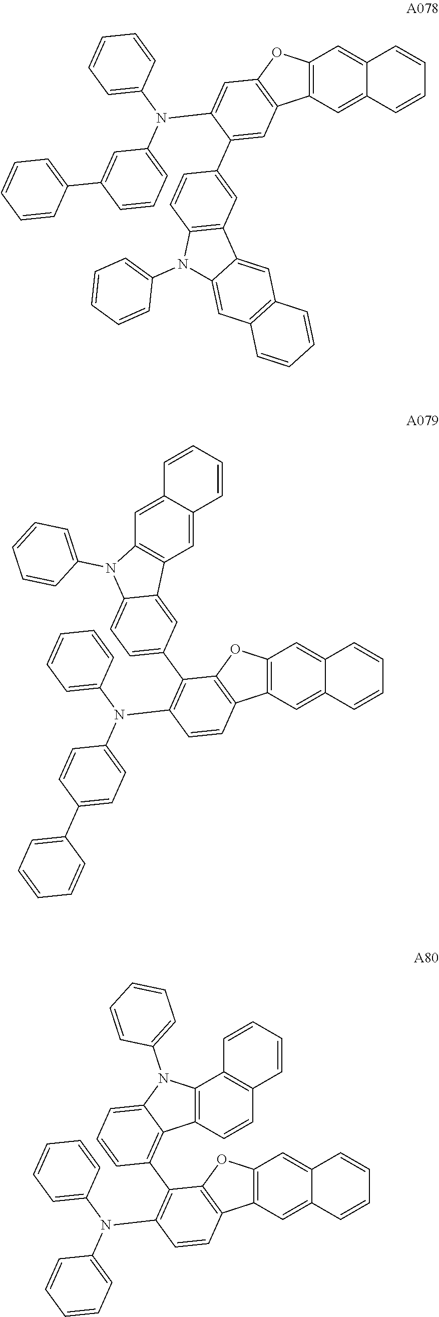 [SMILES: c1ccc(-c2cccc(N(c3ccccc3)c3cc4oc5cc6ccccc6cc5c4cc3-c3ccc4c(c3)c3cc5ccccc5cc3n4-c3ccccc3)c2)cc1 ; c1ccc(-c2ccc(N(c3ccccc3)c3ccc4c(oc5cc6ccccc6cc54)c3-c3ccc4c(c3)c3cc5ccccc5cc3n4-c3ccccc3)cc2)cc1 ; c1ccc(N(c2ccccc2)c2ccc3c(oc4cc5ccccc5cc43)c2-c2cccc3c2c2ccc4ccccc4c2n3-c2ccccc2)cc1]

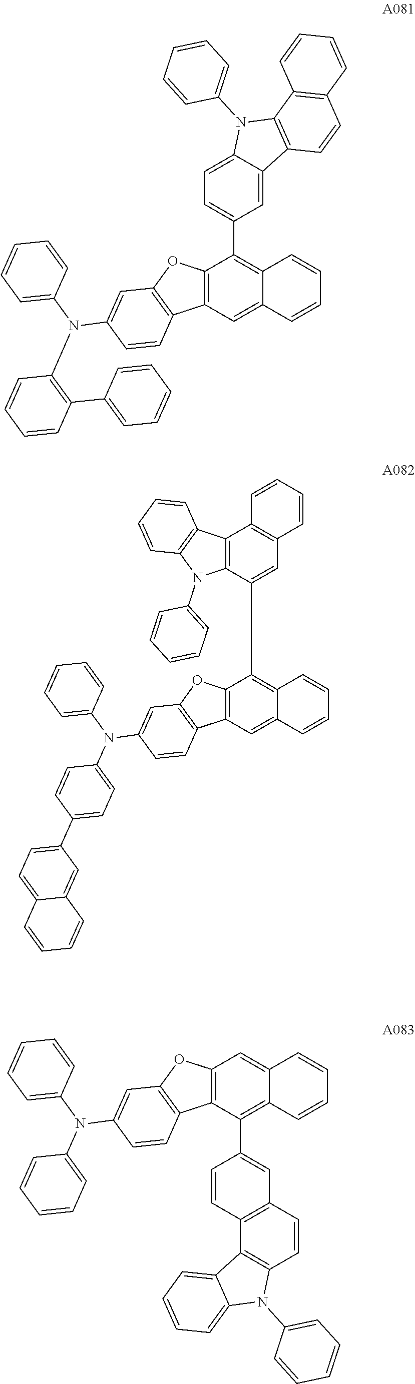 [SMILES: c1ccc(-c2ccccc2N(c2ccccc2)c2ccc3c(c2)oc2c(-c4ccc5c(c4)c4ccc6ccccc6c4n5-c4ccccc4)c4ccccc4cc23)cc1 ; c1ccc(N(c2ccc(-c3ccc4ccccc4c3)cc2)c2ccc3c(c2)oc2c(-c4cc5ccccc5c5c6ccccc6n(-c6ccccc6)c45)c4ccccc4cc23)cc1 ; c1ccc(N(c2ccccc2)c2ccc3c(c2)oc2cc4ccccc4c(-c4ccc5c(ccc6c5c5ccccc5n6-c5ccccc5)c4)c23)cc1]

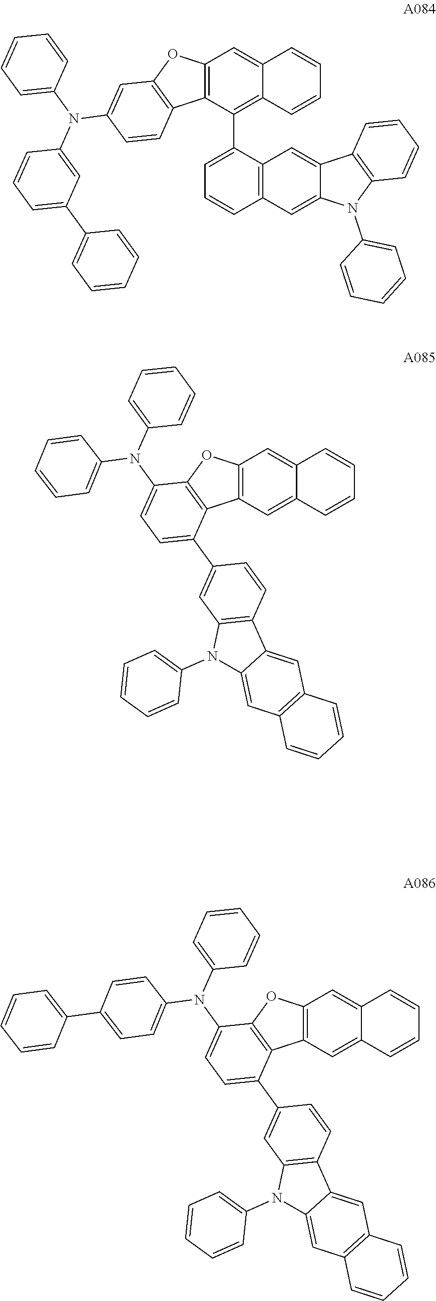 [SMILES: c1ccc(-c2cccc(N(c3ccccc3)c3ccc4c(c3)oc3cc5ccccc5c(-c5cccc6cc7c(cc56)c5ccccc5n7-c5ccccc5)c34)c2)cc1 ; c1ccc(N(c2ccccc2)c2ccc(-c3ccc4c5cc6ccccc6cc5n(-c5ccccc5)c4c3)c3c2oc2cc4ccccc4cc23)cc1 ; c1ccc(-c2ccc(N(c3ccccc3)c3ccc(-c4ccc5c6cc7ccccc7cc6n(-c6ccccc6)c5c4)c4c3oc3cc5ccccc5cc34)cc2)cc1]

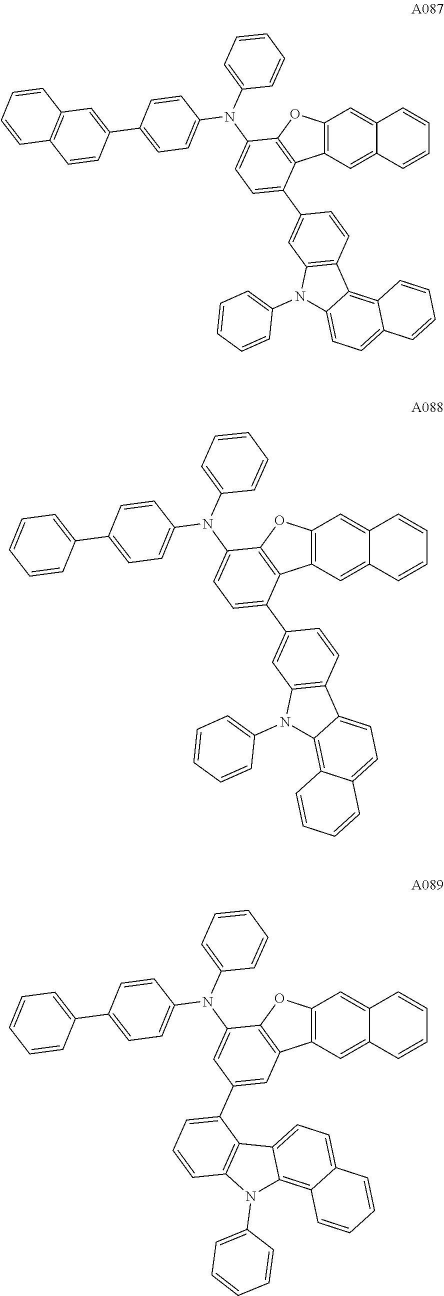 [SMILES: c1ccc(N(c2ccc(-c3ccc4ccccc4c3)cc2)c2ccc(-c3ccc4c5c6ccccc6ccc5n(-c5ccccc5)c4c3)c3c2oc2cc4ccccc4cc23)cc1 ; c1ccc(-c2ccc(N(c3ccccc3)c3ccc(-c4ccc5c6ccc7ccccc7c6n(-c6ccccc6)c5c4)c4c3oc3cc5ccccc5cc34)cc2)cc1 ; c1ccc(-c2ccc(N(c3ccccc3)c3cc(-c4cccc5c4c4ccc6ccccc6c4n5-c4ccccc4)cc4c3oc3cc5ccccc5cc34)cc2)cc1]

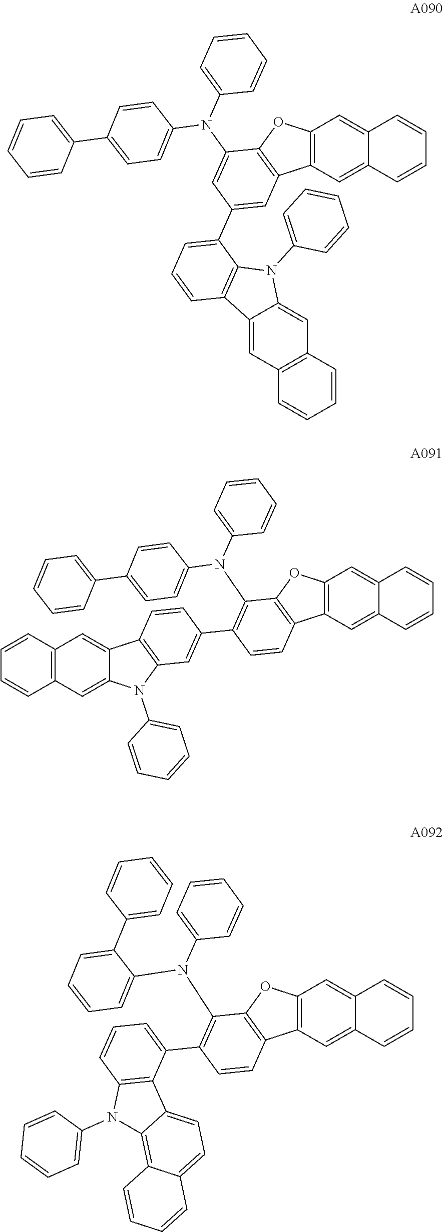 [SMILES: c1ccc(-c2ccc(N(c3ccccc3)c3cc(-c4cccc5c6cc7ccccc7cc6n(-c6ccccc6)c45)cc4c3oc3cc5ccccc5cc34)cc2)cc1 ; c1ccc(-c2ccc(N(c3ccccc3)c3c(-c4ccc5c6cc7ccccc7cc6n(-c6ccccc6)c5c4)ccc4c3oc3cc5ccccc5cc34)cc2)cc1 ; c1ccc(-c2ccccc2N(c2ccccc2)c2c(-c3cccc4c3c3ccc5ccccc5c3n4-c3ccccc3)ccc3c2oc2cc4ccccc4cc23)cc1]

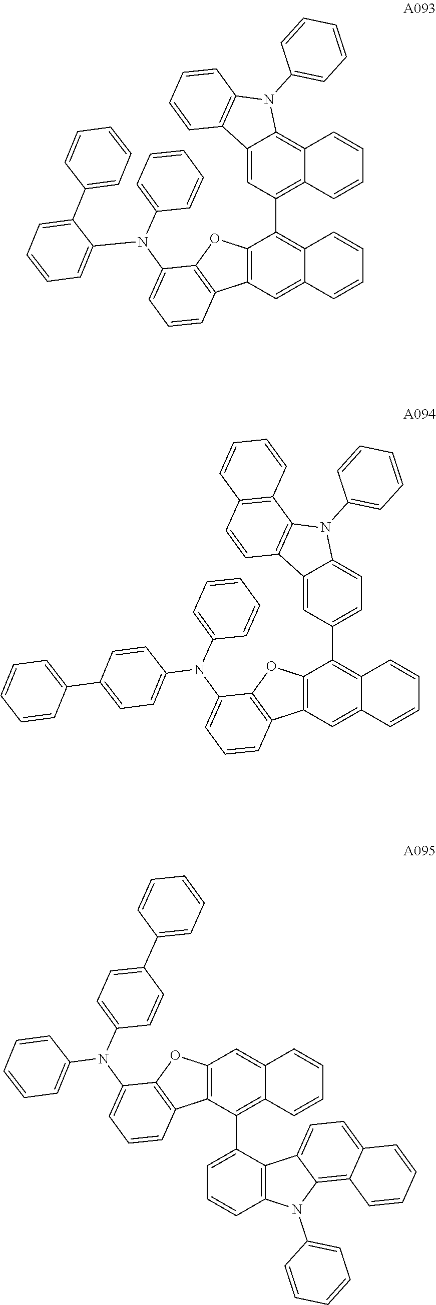 [SMILES: c1ccc(-c2ccccc2N(c2ccccc2)c2cccc3c2oc2c(-c4cc5c6ccccc6n(-c6ccccc6)c5c5ccccc45)c4ccccc4cc23)cc1 ; c1ccc(-c2ccc(N(c3ccccc3)c3cccc4c3oc3c(-c5ccc6c(c5)c5ccc7ccccc7c5n6-c5ccccc5)c5ccccc5cc34)cc2)cc1 ; c1ccc(-c2ccc(N(c3ccccc3)c3cccc4c3oc3cc5ccccc5c(-c5cccc6c5c5ccc7ccccc7c5n6-c5ccccc5)c34)cc2)cc1]

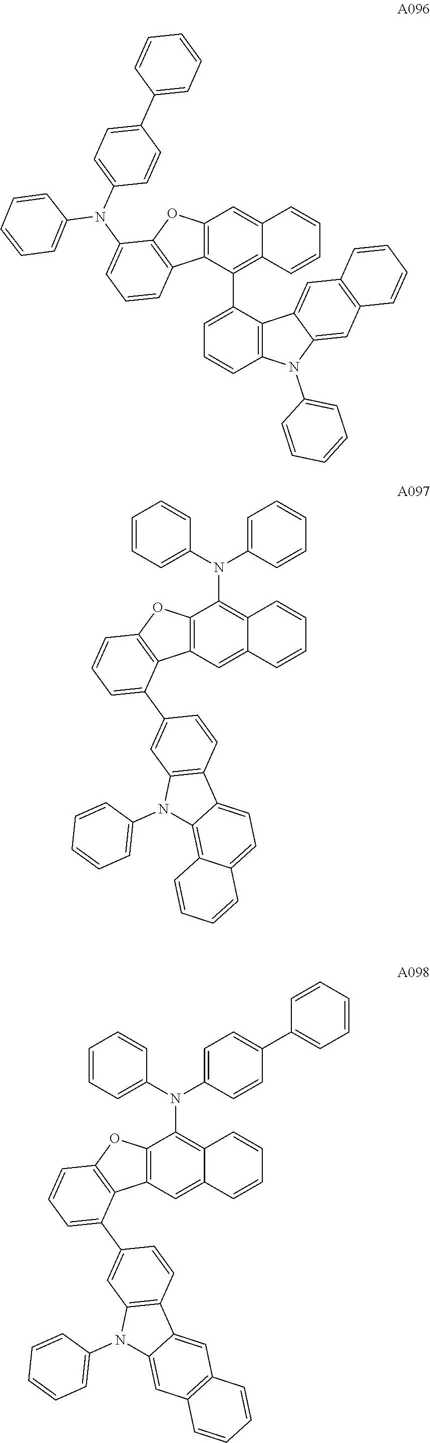 [SMILES: c1ccc(-c2ccc(N(c3ccccc3)c3cccc4c3oc3cc5ccccc5c(-c5cccc6c5c5cc7ccccc7cc5n6-c5ccccc5)c34)cc2)cc1 ; c1ccc(N(c2ccccc2)c2c3ccccc3cc3c2oc2cccc(-c4ccc5c6ccc7ccccc7c6n(-c6ccccc6)c5c4)c23)cc1 ; c1ccc(-c2ccc(N(c3ccccc3)c3c4ccccc4cc4c3oc3cccc(-c5ccc6c7cc8ccccc8cc7n(-c7ccccc7)c6c5)c34)cc2)cc1]

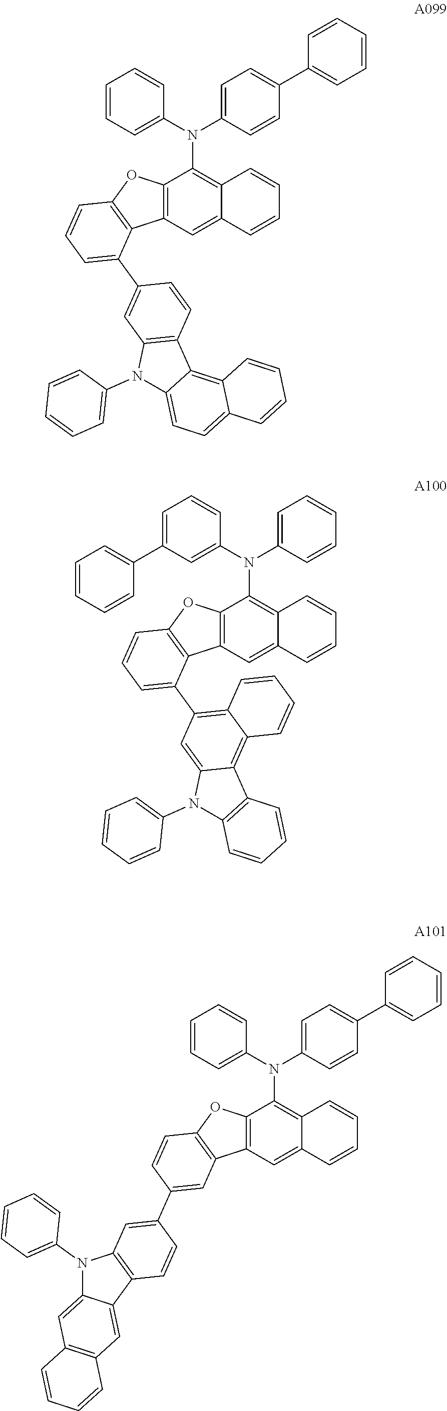 [SMILES: c1ccc(-c2ccc(N(c3ccccc3)c3c4ccccc4cc4c3oc3cccc(-c5ccc6c7c8ccccc8ccc7n(-c7ccccc7)c6c5)c34)cc2)cc1 ; c1ccc(-c2cccc(N(c3ccccc3)c3c4ccccc4cc4c3oc3cccc(-c5cc6c(c7ccccc57)c5ccccc5n6-c5ccccc5)c34)c2)cc1 ; c1ccc(-c2ccc(N(c3ccccc3)c3c4ccccc4cc4c3oc3ccc(-c5ccc6c7cc8ccccc8cc7n(-c7ccccc7)c6c5)cc34)cc2)cc1]

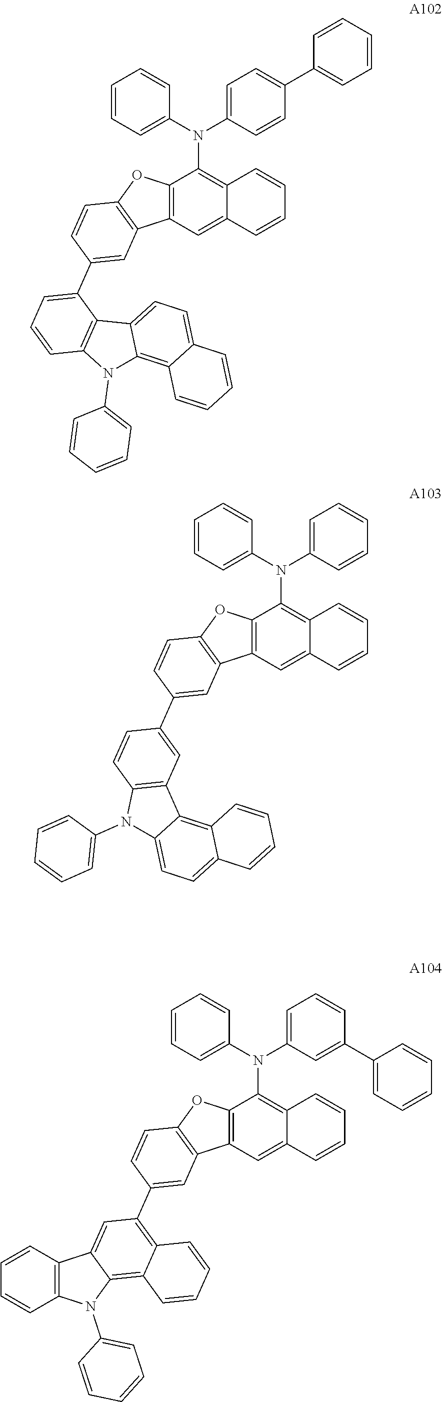 [SMILES: c1ccc(-c2ccc(N(c3ccccc3)c3c4ccccc4cc4c3oc3ccc(-c5cccc6c5c5ccc7ccccc7c5n6-c5ccccc5)cc34)cc2)cc1 ; c1ccc(N(c2ccccc2)c2c3ccccc3cc3c2oc2ccc(-c4ccc5c(c4)c4c6ccccc6ccc4n5-c4ccccc4)cc23)cc1 ; c1ccc(-c2cccc(N(c3ccccc3)c3c4ccccc4cc4c3oc3ccc(-c5cc6c7ccccc7n(-c7ccccc7)c6c6ccccc56)cc34)c2)cc1]

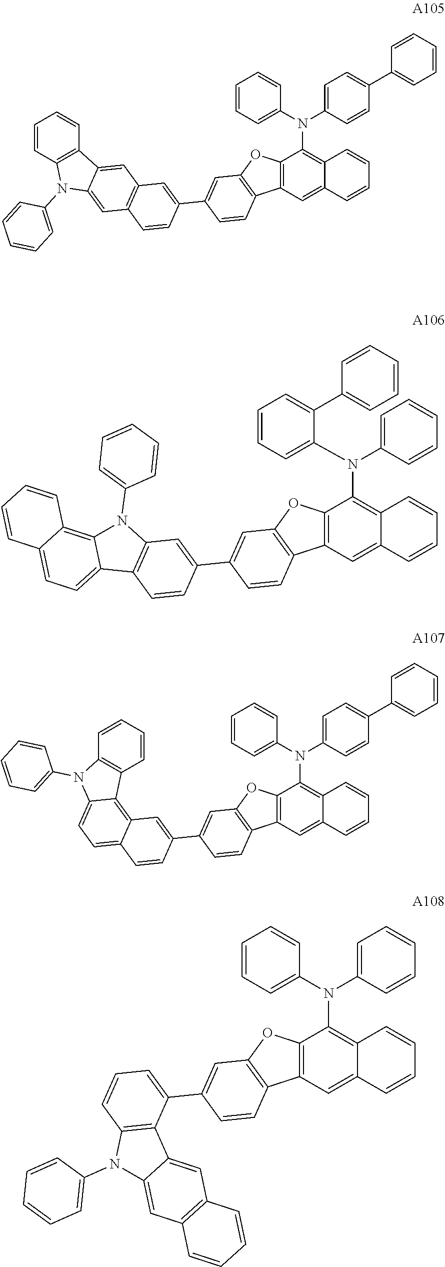 [SMILES: c1ccc(-c2ccc(N(c3ccccc3)c3c4ccccc4cc4c3oc3cc(-c5ccc6cc7c(cc6c5)c5ccccc5n7-c5ccccc5)ccc34)cc2)cc1 ; c1ccc(-c2ccccc2N(c2ccccc2)c2c3ccccc3cc3c2oc2cc(-c4ccc5c6ccc7ccccc7c6n(-c6ccccc6)c5c4)ccc23)cc1 ; c1ccc(-c2ccc(N(c3ccccc3)c3c4ccccc4cc4c3oc3cc(-c5ccc6ccc7c(c6c5)c5ccccc5n7-c5ccccc5)ccc34)cc2)cc1 ; c1ccc(N(c2ccccc2)c2c3ccccc3cc3c2oc2cc(-c4cccc5c4c4cc6ccccc6cc4n5-c4ccccc4)ccc23)cc1]

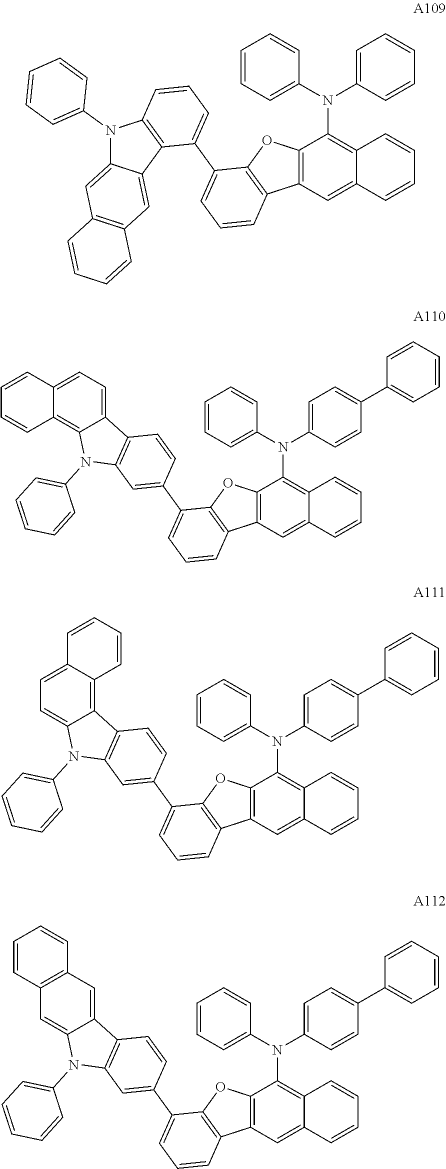 [SMILES: c1ccc(N(c2ccccc2)c2c3ccccc3cc3c2oc2c(-c4cccc5c4c4cc6ccccc6cc4n5-c4ccccc4)cccc23)cc1 ; c1ccc(-c2ccc(N(c3ccccc3)c3c4ccccc4cc4c3oc3c(-c5ccc6c7ccc8ccccc8c7n(-c7ccccc7)c6c5)cccc34)cc2)cc1 ; c1ccc(-c2ccc(N(c3ccccc3)c3c4ccccc4cc4c3oc3c(-c5ccc6c7c8ccccc8ccc7n(-c7ccccc7)c6c5)cccc34)cc2)cc1 ; c1ccc(-c2ccc(N(c3ccccc3)c3c4ccccc4cc4c3oc3c(-c5ccc6c7cc8ccccc8cc7n(-c7ccccc7)c6c5)cccc34)cc2)cc1]

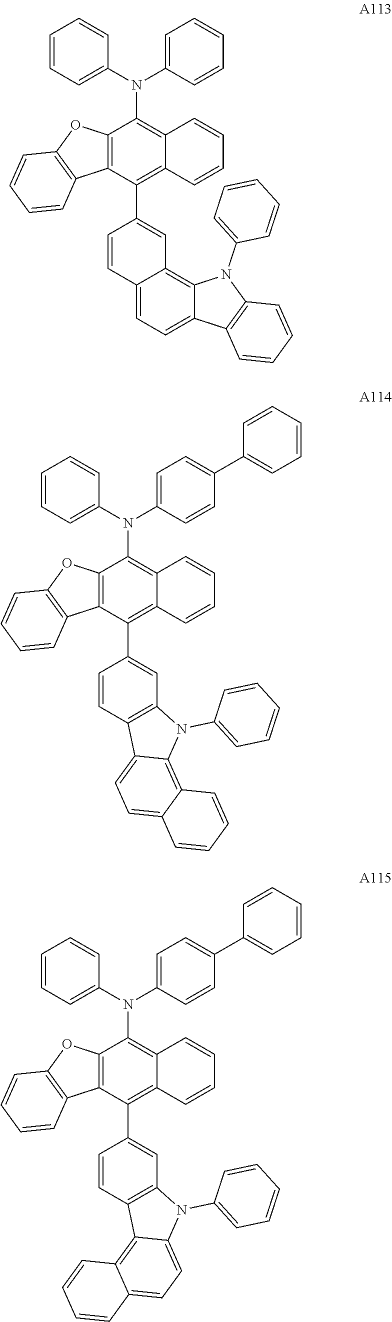

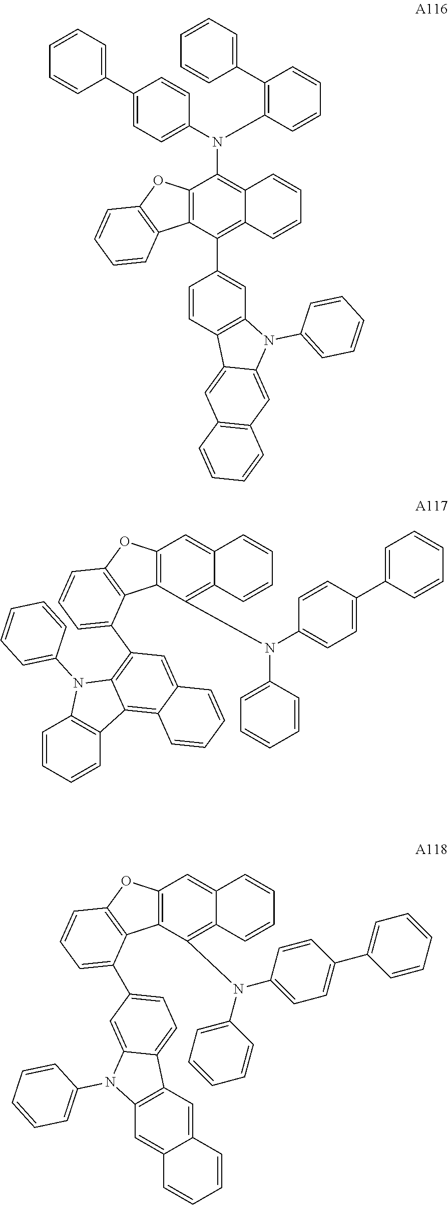 [SMILES: c1ccc(-c2ccc(N(c3ccccc3-c3ccccc3)c3c4ccccc4c(-c4ccc5c6cc7ccccc7cc6n(-c6ccccc6)c5c4)c4c3oc3ccccc34)cc2)cc1 ; c1ccc(-c2ccc(N(c3ccccc3)c3c4ccccc4cc4oc5cccc(-c6cc7ccccc7c7c8ccccc8n(-c8ccccc8)c67)c5c34)cc2)cc1 ; c1ccc(-c2ccc(N(c3ccccc3)c3c4ccccc4cc4oc5cccc(-c6ccc7c8cc9ccccc9cc8n(-c8ccccc8)c7c6)c5c34)cc2)cc1]

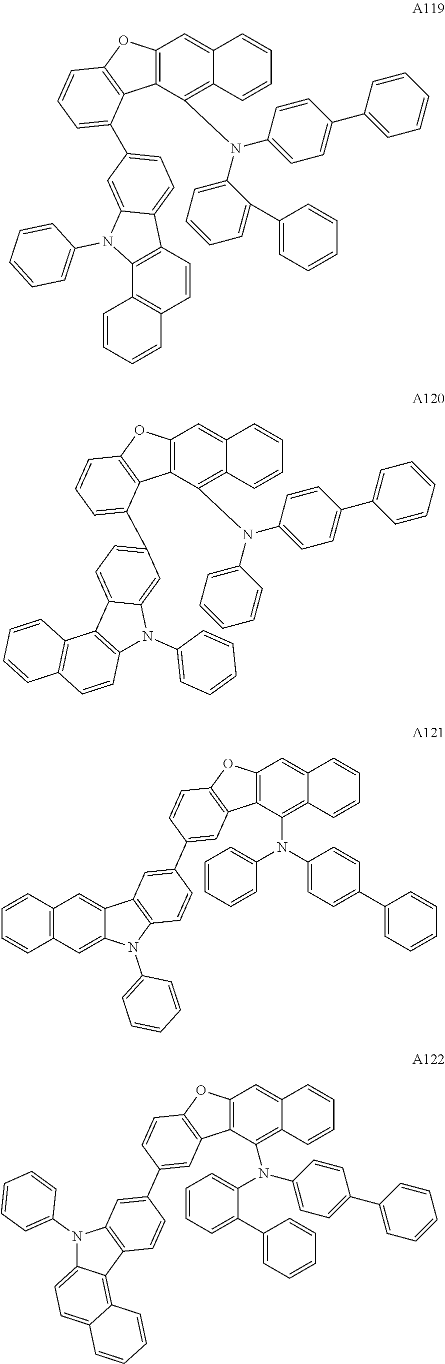 [SMILES: c1ccc(-c2ccc(N(c3ccccc3-c3ccccc3)c3c4ccccc4cc4oc5cccc(-c6ccc7c8ccc9ccccc9c8n(-c8ccccc8)c7c6)c5c34)cc2)cc1 ; c1ccc(-c2ccc(N(c3ccccc3)c3c4ccccc4cc4oc5cccc(-c6ccc7c8c9ccccc9ccc8n(-c8ccccc8)c7c6)c5c34)cc2)cc1 ; c1ccc(-c2ccc(N(c3ccccc3)c3c4ccccc4cc4oc5ccc(-c6ccc7c(c6)c6cc8ccccc8cc6n7-c6ccccc6)cc5c34)cc2)cc1 ; c1ccc(-c2ccc(N(c3ccccc3-c3ccccc3)c3c4ccccc4cc4oc5ccc(-c6ccc7c8c9ccccc9ccc8n(-c8ccccc8)c7c6)cc5c34)cc2)cc1]

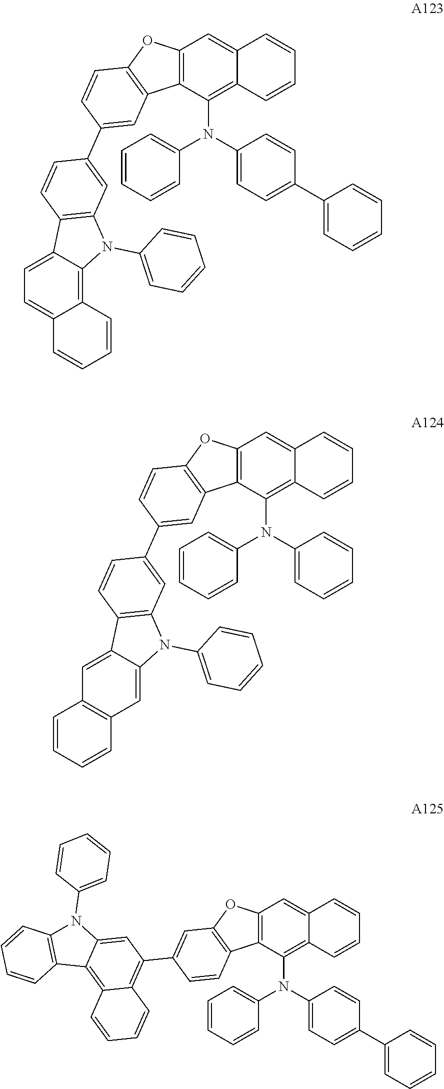 [SMILES: c1ccc(-c2ccc(N(c3ccccc3)c3c4ccccc4cc4oc5ccc(-c6ccc7c8ccc9ccccc9c8n(-c8ccccc8)c7c6)cc5c34)cc2)cc1 ; c1ccc(N(c2ccccc2)c2c3ccccc3cc3oc4ccc(-c5ccc6c7cc8ccccc8cc7n(-c7ccccc7)c6c5)cc4c23)cc1 ; c1ccc(-c2ccc(N(c3ccccc3)c3c4ccccc4cc4oc5cc(-c6cc7c(c8ccccc68)c6ccccc6n7-c6ccccc6)ccc5c34)cc2)cc1]

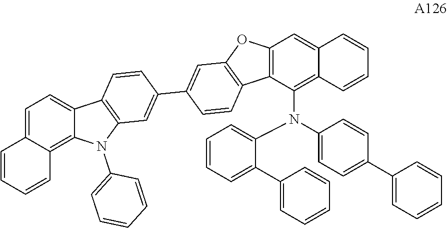 [SMILES: c1ccc(-c2ccc(N(c3ccccc3-c3ccccc3)c3c4ccccc4cc4oc5cc(-c6ccc7c8ccc9ccccc9c8n(-c8ccccc8)c7c6)ccc5c34)cc2)cc1]

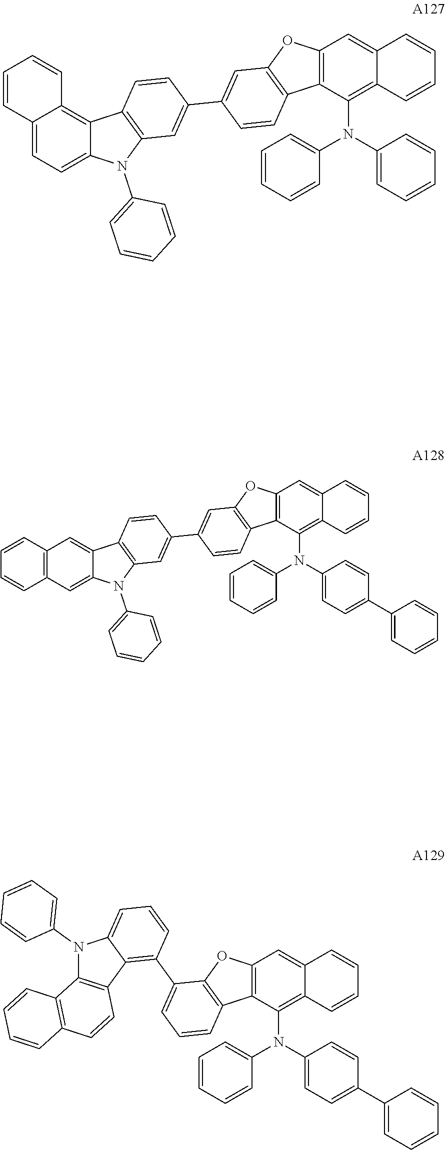 [SMILES: c1ccc(N(c2ccccc2)c2c3ccccc3cc3oc4cc(-c5ccc6c7c8ccccc8ccc7n(-c7ccccc7)c6c5)ccc4c23)cc1 ; c1ccc(-c2ccc(N(c3ccccc3)c3c4ccccc4cc4oc5cc(-c6ccc7c8cc9ccccc9cc8n(-c8ccccc8)c7c6)ccc5c34)cc2)cc1 ; c1ccc(-c2ccc(N(c3ccccc3)c3c4ccccc4cc4oc5c(-c6cccc7c6c6ccc8ccccc8c6n7-c6ccccc6)cccc5c34)cc2)cc1]

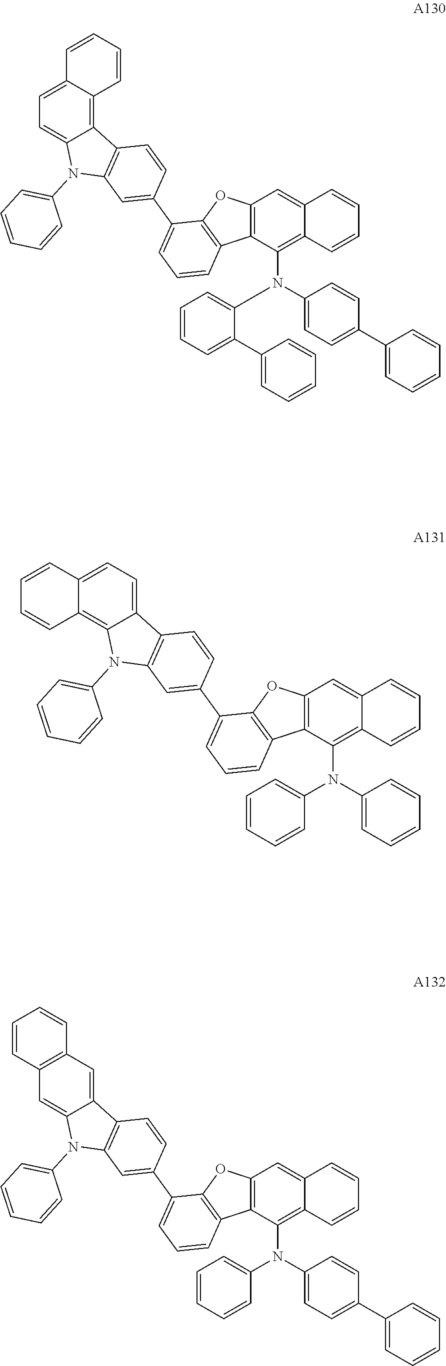 [SMILES: c1ccc(-c2ccc(N(c3ccccc3-c3ccccc3)c3c4ccccc4cc4oc5c(-c6ccc7c8c9ccccc9ccc8n(-c8ccccc8)c7c6)cccc5c34)cc2)cc1 ; c1ccc(N(c2ccccc2)c2c3ccccc3cc3oc4c(-c5ccc6c7ccc8ccccc8c7n(-c7ccccc7)c6c5)cccc4c23)cc1 ; c1ccc(-c2ccc(N(c3ccccc3)c3c4ccccc4cc4oc5c(-c6ccc7c8cc9ccccc9cc8n(-c8ccccc8)c7c6)cccc5c34)cc2)cc1]

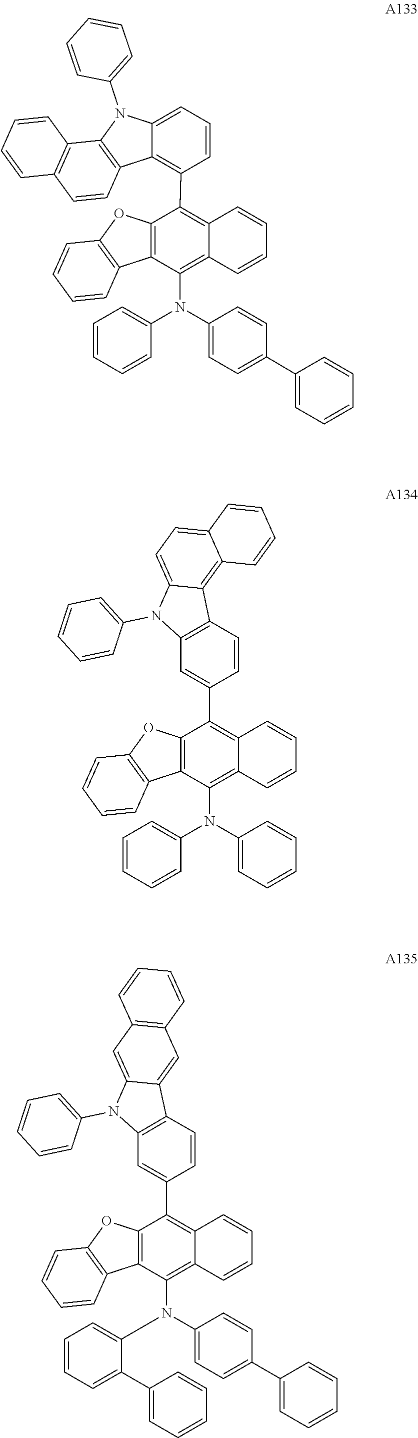 [SMILES: c1ccc(-c2ccc(N(c3ccccc3)c3c4ccccc4c(-c4cccc5c4c4ccc6ccccc6c4n5-c4ccccc4)c4oc5ccccc5c34)cc2)cc1 ; c1ccc(N(c2ccccc2)c2c3ccccc3c(-c3ccc4c5c6ccccc6ccc5n(-c5ccccc5)c4c3)c3oc4ccccc4c23)cc1 ; c1ccc(-c2ccc(N(c3ccccc3-c3ccccc3)c3c4ccccc4c(-c4ccc5c6cc7ccccc7cc6n(-c6ccccc6)c5c4)c4oc5ccccc5c34)cc2)cc1]

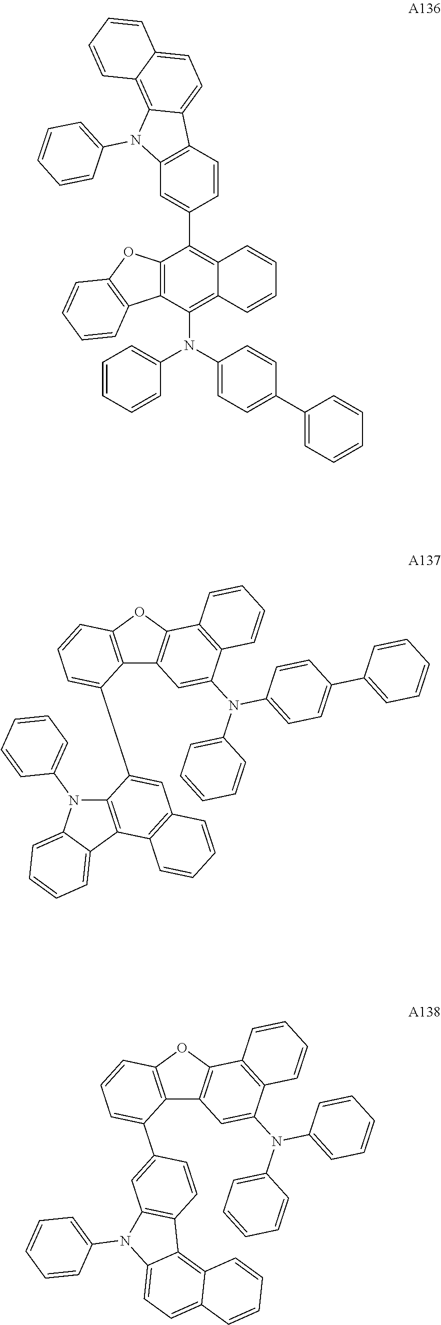 [SMILES: c1ccc(-c2ccc(N(c3ccccc3)c3c4ccccc4c(-c4ccc5c6ccc7ccccc7c6n(-c6ccccc6)c5c4)c4oc5ccccc5c34)cc2)cc1 ; c1ccc(-c2ccc(N(c3ccccc3)c3cc4c(oc5cccc(-c6cc7ccccc7c7c8ccccc8n(-c8ccccc8)c67)c54)c4ccccc34)cc2)cc1 ; c1ccc(N(c2ccccc2)c2cc3c(oc4cccc(-c5ccc6c7c8ccccc8ccc7n(-c7ccccc7)c6c5)c43)c3ccccc23)cc1]

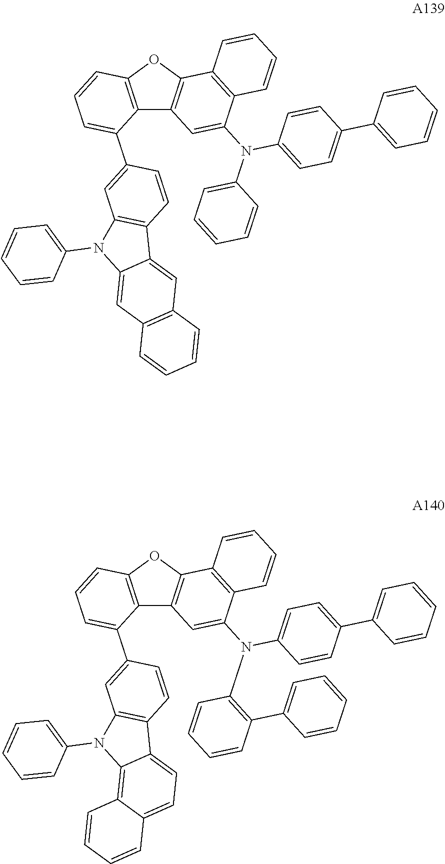 [SMILES: c1ccc(-c2ccc(N(c3ccccc3)c3cc4c(oc5cccc(-c6ccc7c8cc9ccccc9cc8n(-c8ccccc8)c7c6)c54)c4ccccc34)cc2)cc1 ; c1ccc(-c2ccc(N(c3ccccc3-c3ccccc3)c3cc4c(oc5cccc(-c6ccc7c8ccc9ccccc9c8n(-c8ccccc8)c7c6)c54)c4ccccc34)cc2)cc1]

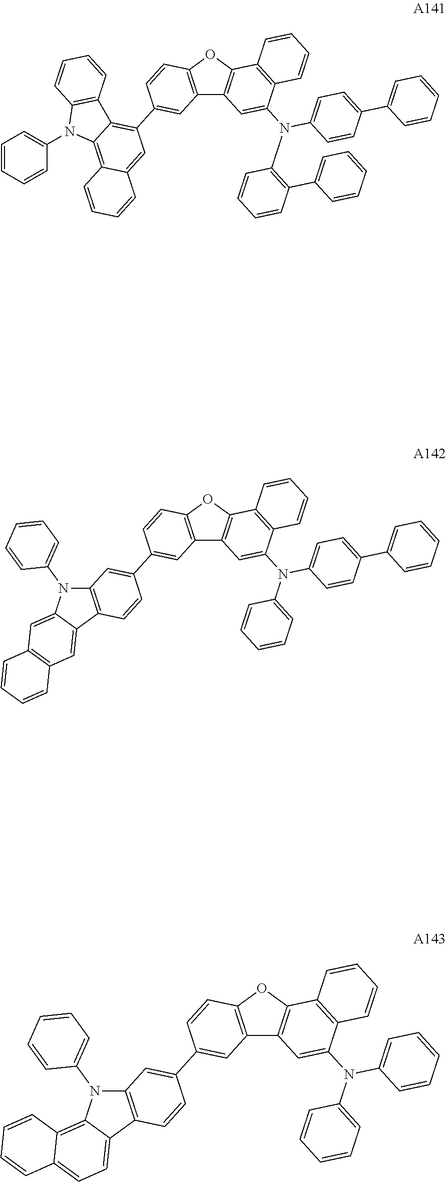 [SMILES: c1ccc(-c2ccc(N(c3ccccc3-c3ccccc3)c3cc4c5cc(-c6cc7ccccc7c7c6c6ccccc6n7-c6ccccc6)ccc5oc4c4ccccc34)cc2)cc1 ; c1ccc(-c2ccc(N(c3ccccc3)c3cc4c5cc(-c6ccc7c8cc9ccccc9cc8n(-c8ccccc8)c7c6)ccc5oc4c4ccccc34)cc2)cc1 ; c1ccc(N(c2ccccc2)c2cc3c4cc(-c5ccc6c7ccc8ccccc8c7n(-c7ccccc7)c6c5)ccc4oc3c3ccccc23)cc1]

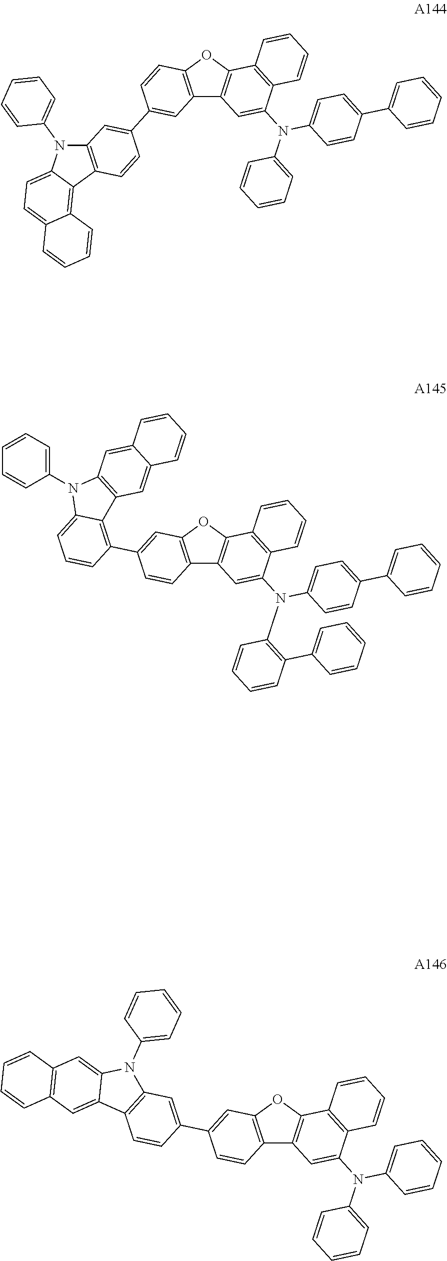 [SMILES: c1ccc(-c2ccc(N(c3ccccc3)c3cc4c5cc(-c6ccc7c8c9ccccc9ccc8n(-c8ccccc8)c7c6)ccc5oc4c4ccccc34)cc2)cc1 ; c1ccc(-c2ccc(N(c3ccccc3-c3ccccc3)c3cc4c5ccc(-c6cccc7c6c6cc8ccccc8cc6n7-c6ccccc6)cc5oc4c4ccccc34)cc2)cc1 ; c1ccc(N(c2ccccc2)c2cc3c4ccc(-c5ccc6c7cc8ccccc8cc7n(-c7ccccc7)c6c5)cc4oc3c3ccccc23)cc1]

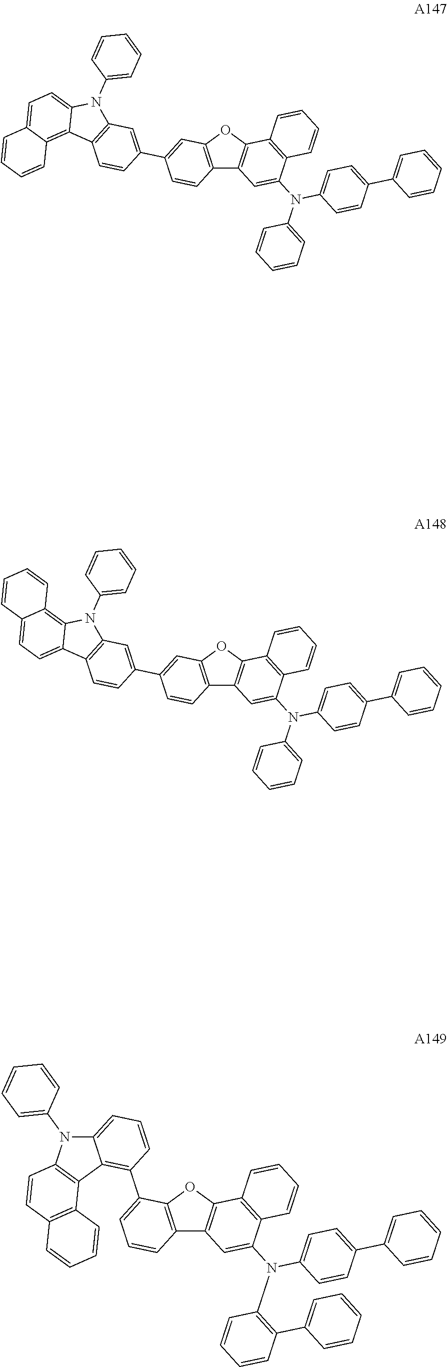 [SMILES: c1ccc(-c2ccc(N(c3ccccc3)c3cc4c5ccc(-c6ccc7c8c9ccccc9ccc8n(-c8ccccc8)c7c6)cc5oc4c4ccccc34)cc2)cc1 ; c1ccc(-c2ccc(N(c3ccccc3)c3cc4c5ccc(-c6ccc7c8ccc9ccccc9c8n(-c8ccccc8)c7c6)cc5oc4c4ccccc34)cc2)cc1 ; c1ccc(-c2ccc(N(c3ccccc3-c3ccccc3)c3cc4c5cccc(-c6cccc7c6c6c8ccccc8ccc6n7-c6ccccc6)c5oc4c4ccccc34)cc2)cc1]

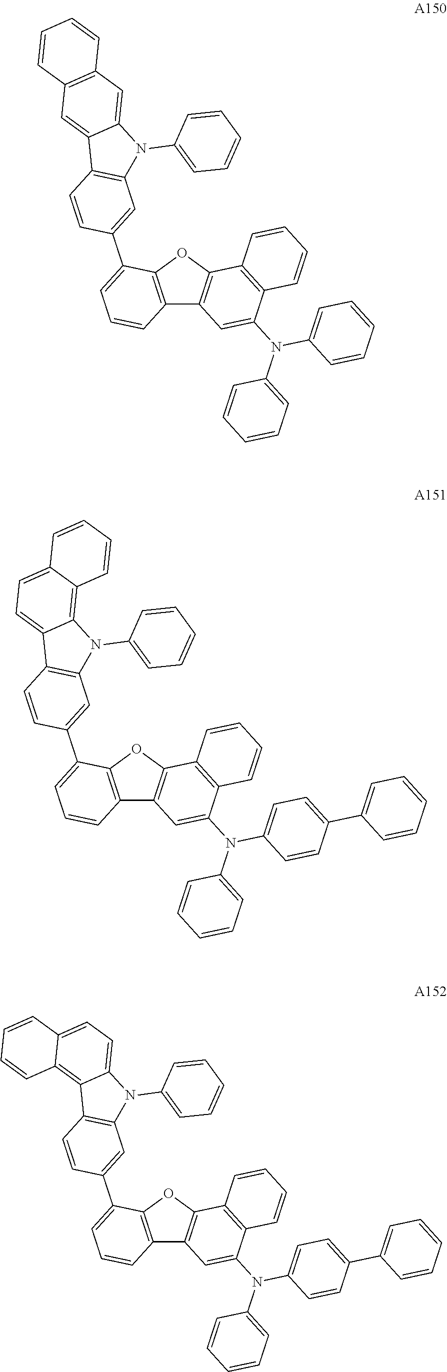 [SMILES: c1ccc(N(c2ccccc2)c2cc3c4cccc(-c5ccc6c7cc8ccccc8cc7n(-c7ccccc7)c6c5)c4oc3c3ccccc23)cc1 ; c1ccc(-c2ccc(N(c3ccccc3)c3cc4c5cccc(-c6ccc7c8ccc9ccccc9c8n(-c8ccccc8)c7c6)c5oc4c4ccccc34)cc2)cc1 ; c1ccc(-c2ccc(N(c3ccccc3)c3cc4c5cccc(-c6ccc7c8c9ccccc9ccc8n(-c8ccccc8)c7c6)c5oc4c4ccccc34)cc2)cc1]

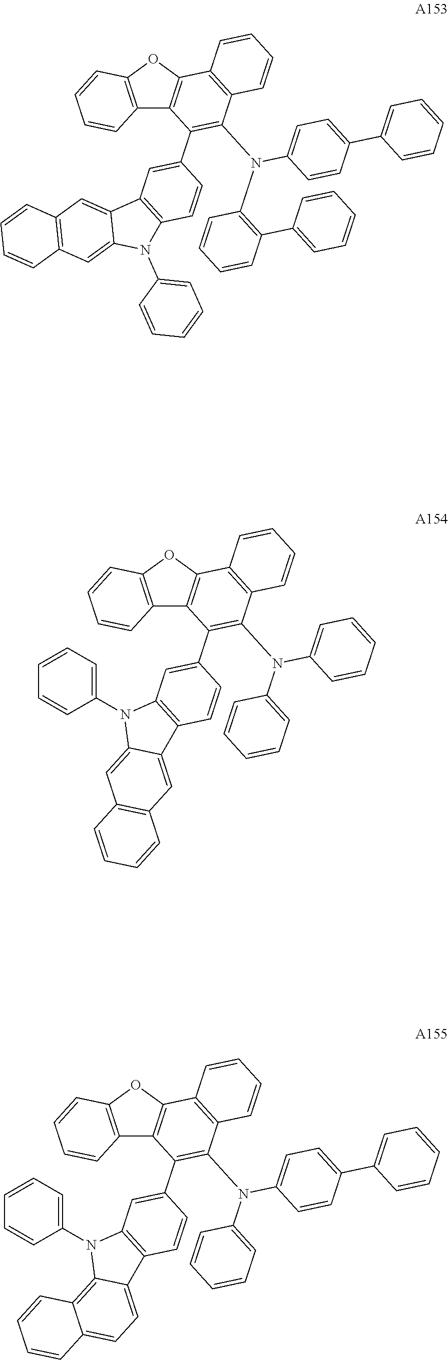 [SMILES: c1ccc(-c2ccc(N(c3ccccc3-c3ccccc3)c3c(-c4ccc5c(c4)c4cc6ccccc6cc4n5-c4ccccc4)c4c5ccccc5oc4c4ccccc34)cc2)cc1 ; c1ccc(N(c2ccccc2)c2c(-c3ccc4c5cc6ccccc6cc5n(-c5ccccc5)c4c3)c3c4ccccc4oc3c3ccccc23)cc1 ; c1ccc(-c2ccc(N(c3ccccc3)c3c(-c4ccc5c6ccc7ccccc7c6n(-c6ccccc6)c5c4)c4c5ccccc5oc4c4ccccc34)cc2)cc1]

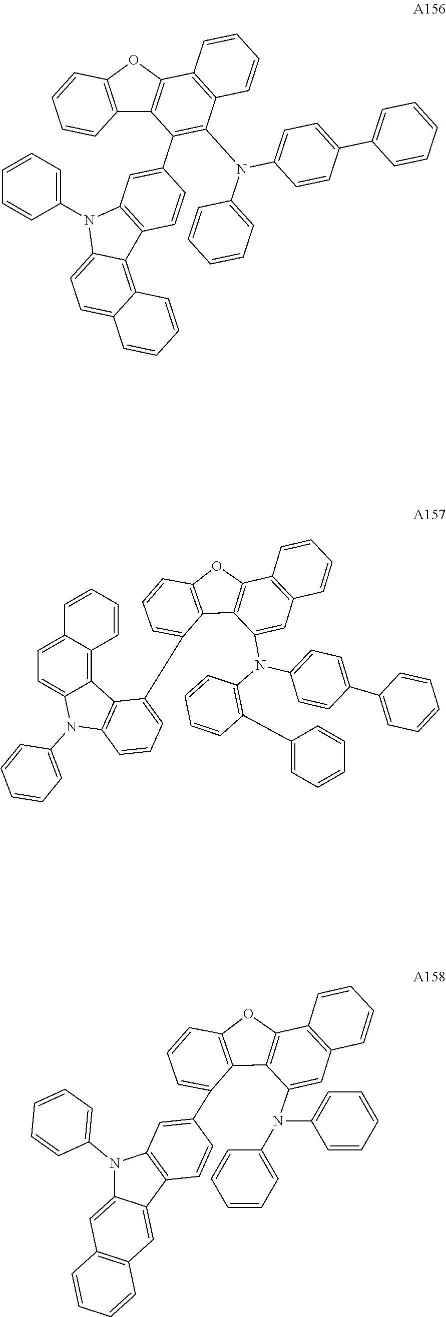 [SMILES: c1ccc(-c2ccc(N(c3ccccc3)c3c(-c4ccc5c6c7ccccc7ccc6n(-c6ccccc6)c5c4)c4c5ccccc5oc4c4ccccc34)cc2)cc1 ; c1ccc(-c2ccc(N(c3ccccc3-c3ccccc3)c3cc4ccccc4c4oc5cccc(-c6cccc7c6c6c8ccccc8ccc6n7-c6ccccc6)c5c34)cc2)cc1 ; c1ccc(N(c2ccccc2)c2cc3ccccc3c3oc4cccc(-c5ccc6c7cc8ccccc8cc7n(-c7ccccc7)c6c5)c4c23)cc1]

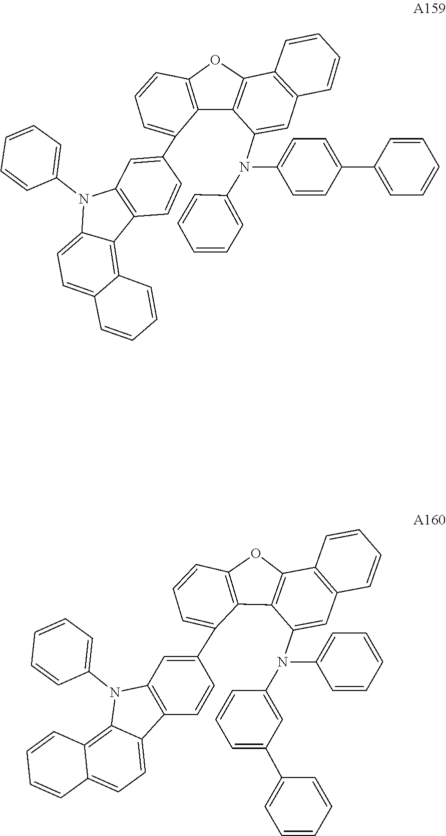 [SMILES: c1ccc(-c2ccc(N(c3ccccc3)c3cc4ccccc4c4oc5cccc(-c6ccc7c8c9ccccc9ccc8n(-c8ccccc8)c7c6)c5c34)cc2)cc1 ; c1ccc(-c2cccc(N(c3ccccc3)c3cc4ccccc4c4oc5cccc(-c6ccc7c8ccc9ccccc9c8n(-c8ccccc8)c7c6)c5c34)c2)cc1]

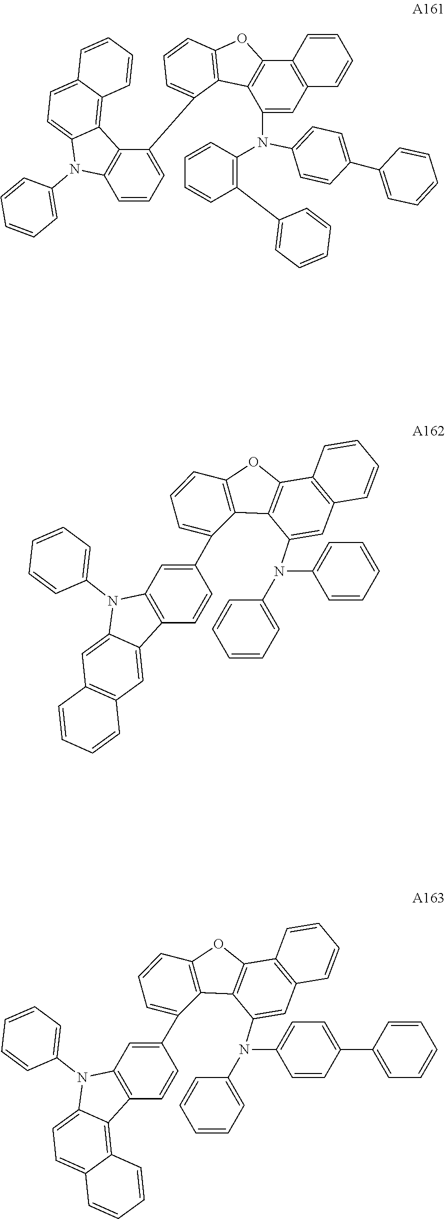 [SMILES: c1ccc(-c2ccc(N(c3ccccc3-c3ccccc3)c3cc4ccccc4c4oc5cccc(-c6cccc7c6c6c8ccccc8ccc6n7-c6ccccc6)c5c34)cc2)cc1 ; c1ccc(N(c2ccccc2)c2cc3ccccc3c3oc4cccc(-c5ccc6c7cc8ccccc8cc7n(-c7ccccc7)c6c5)c4c23)cc1 ; c1ccc(-c2ccc(N(c3ccccc3)c3cc4ccccc4c4oc5cccc(-c6ccc7c8c9ccccc9ccc8n(-c8ccccc8)c7c6)c5c34)cc2)cc1]

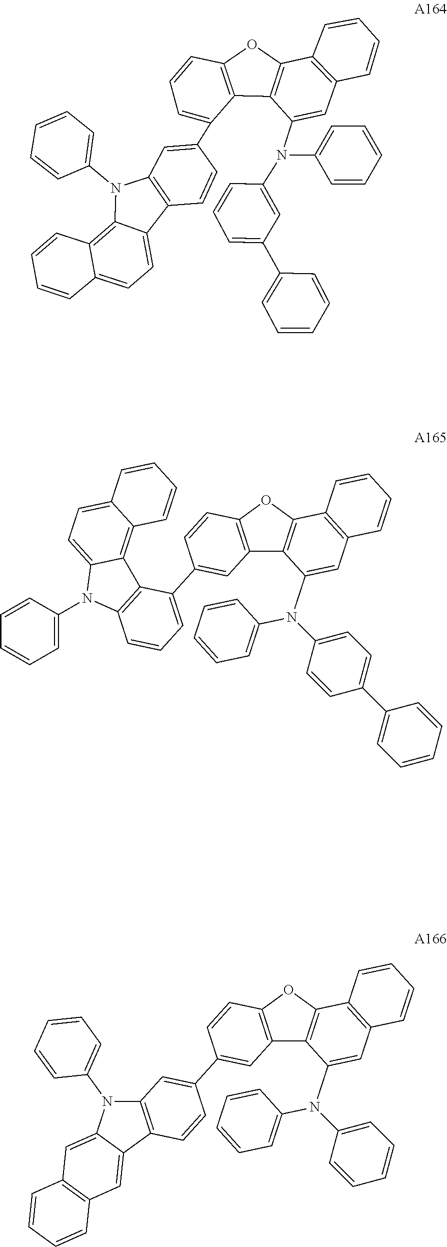 [SMILES: c1ccc(-c2cccc(N(c3ccccc3)c3cc4ccccc4c4oc5cccc(-c6ccc7c8ccc9ccccc9c8n(-c8ccccc8)c7c6)c5c34)c2)cc1 ; c1ccc(-c2ccc(N(c3ccccc3)c3cc4ccccc4c4oc5ccc(-c6cccc7c6c6c8ccccc8ccc6n7-c6ccccc6)cc5c34)cc2)cc1 ; c1ccc(N(c2ccccc2)c2cc3ccccc3c3oc4ccc(-c5ccc6c7cc8ccccc8cc7n(-c7ccccc7)c6c5)cc4c23)cc1]

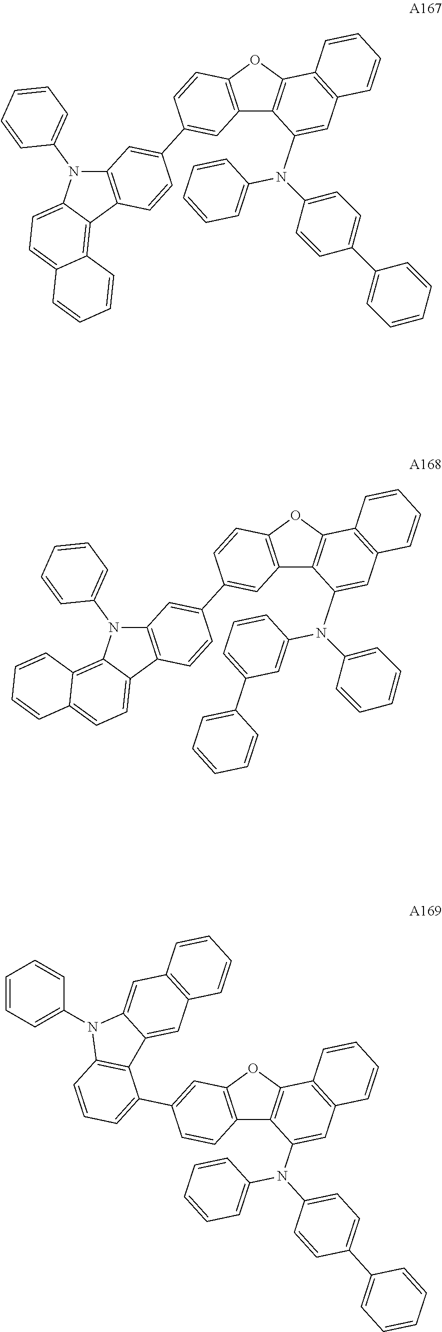 [SMILES: c1ccc(-c2ccc(N(c3ccccc3)c3cc4ccccc4c4oc5ccc(-c6ccc7c8c9ccccc9ccc8n(-c8ccccc8)c7c6)cc5c34)cc2)cc1 ; c1ccc(-c2cccc(N(c3ccccc3)c3cc4ccccc4c4oc5ccc(-c6ccc7c8ccc9ccccc9c8n(-c8ccccc8)c7c6)cc5c34)c2)cc1 ; c1ccc(-c2ccc(N(c3ccccc3)c3cc4ccccc4c4oc5cc(-c6cccc7c6c6cc8ccccc8cc6n7-c6ccccc6)ccc5c34)cc2)cc1]

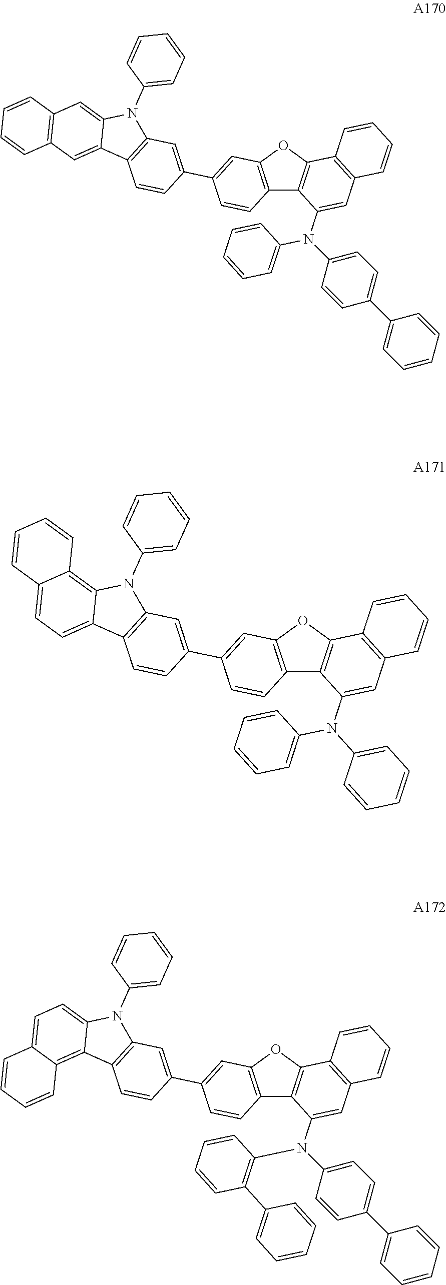 [SMILES: c1ccc(-c2ccc(N(c3ccccc3)c3cc4ccccc4c4oc5cc(-c6ccc7c8cc9ccccc9cc8n(-c8ccccc8)c7c6)ccc5c34)cc2)cc1 ; c1ccc(N(c2ccccc2)c2cc3ccccc3c3oc4cc(-c5ccc6c7ccc8ccccc8c7n(-c7ccccc7)c6c5)ccc4c23)cc1 ; c1ccc(-c2ccc(N(c3ccccc3-c3ccccc3)c3cc4ccccc4c4oc5cc(-c6ccc7c8c9ccccc9ccc8n(-c8ccccc8)c7c6)ccc5c34)cc2)cc1]

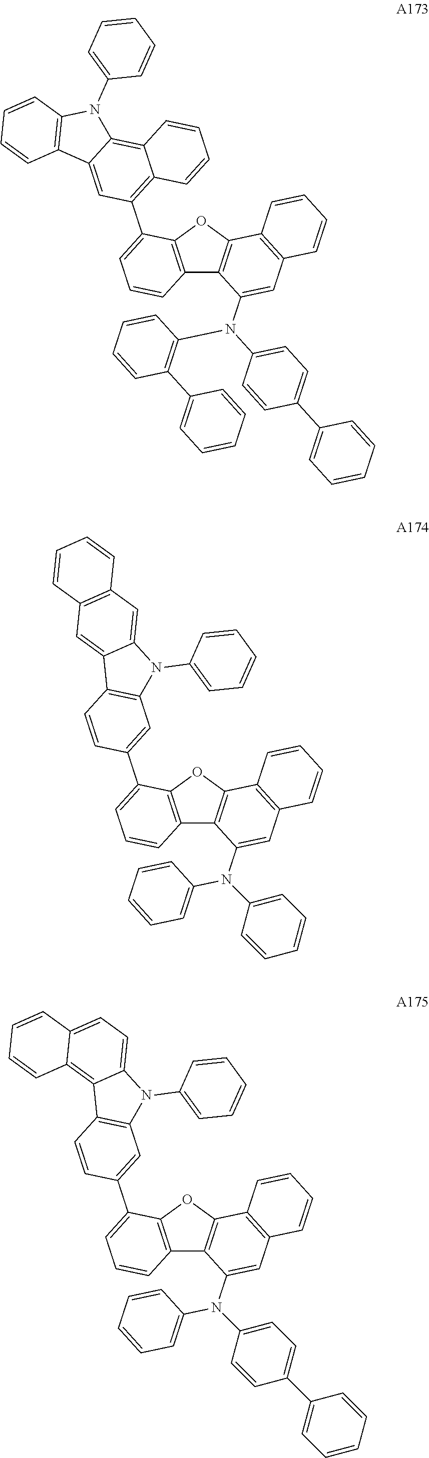 [SMILES: c1ccc(-c2ccc(N(c3ccccc3-c3ccccc3)c3cc4ccccc4c4oc5c(-c6cc7c8ccccc8n(-c8ccccc8)c7c7ccccc67)cccc5c34)cc2)cc1 ; c1ccc(N(c2ccccc2)c2cc3ccccc3c3oc4c(-c5ccc6c7cc8ccccc8cc7n(-c7ccccc7)c6c5)cccc4c23)cc1 ; c1ccc(-c2ccc(N(c3ccccc3)c3cc4ccccc4c4oc5c(-c6ccc7c8c9ccccc9ccc8n(-c8ccccc8)c7c6)cccc5c34)cc2)cc1]

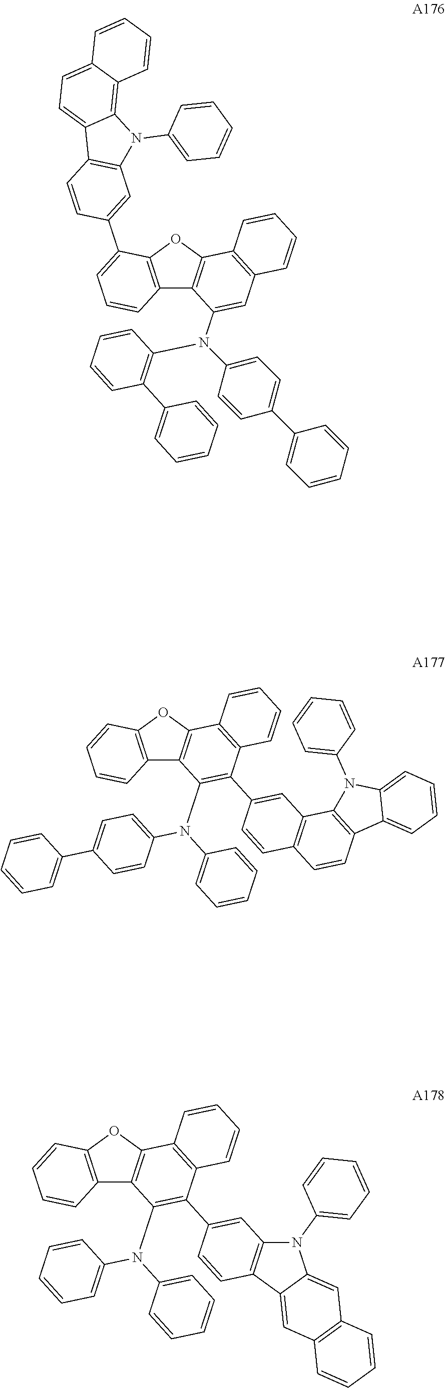 [SMILES: c1ccc(-c2ccc(N(c3ccccc3-c3ccccc3)c3cc4ccccc4c4oc5c(-c6ccc7c8ccc9ccccc9c8n(-c8ccccc8)c7c6)cccc5c34)cc2)cc1 ; c1ccc(-c2ccc(N(c3ccccc3)c3c(-c4ccc5ccc6c7ccccc7n(-c7ccccc7)c6c5c4)c4ccccc4c4oc5ccccc5c34)cc2)cc1 ; c1ccc(N(c2ccccc2)c2c(-c3ccc4c5cc6ccccc6cc5n(-c5ccccc5)c4c3)c3ccccc3c3oc4ccccc4c23)cc1]

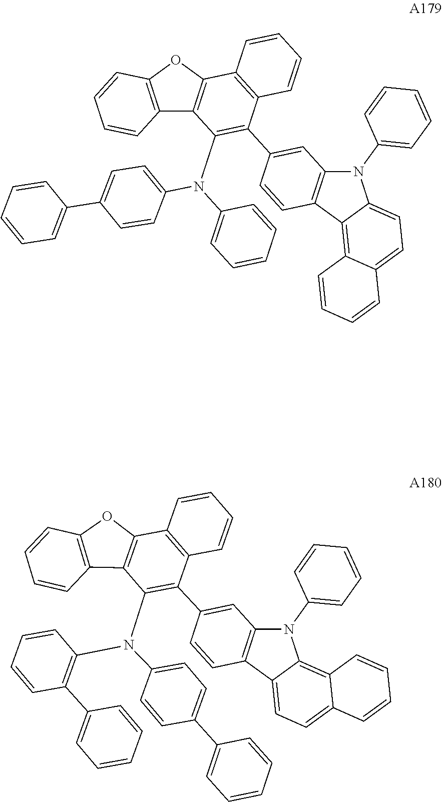 [SMILES: c1ccc(-c2ccc(N(c3ccccc3)c3c(-c4ccc5c6c7ccccc7ccc6n(-c6ccccc6)c5c4)c4ccccc4c4oc5ccccc5c34)cc2)cc1 ; c1ccc(-c2ccc(N(c3ccccc3-c3ccccc3)c3c(-c4ccc5c6ccc7ccccc7c6n(-c6ccccc6)c5c4)c4ccccc4c4oc5ccccc5c34)cc2)cc1]

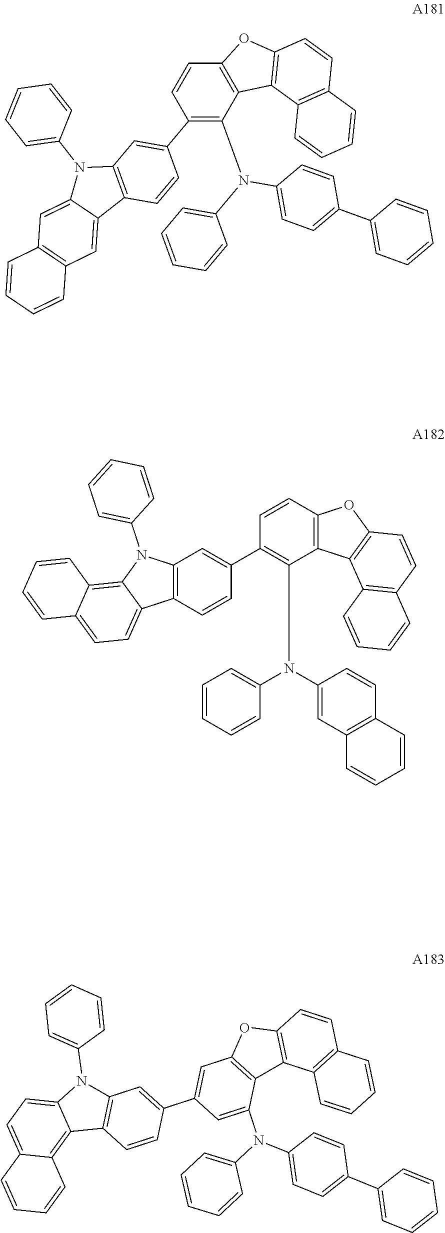 [SMILES: c1ccc(-c2ccc(N(c3ccccc3)c3c(-c4ccc5c6cc7ccccc7cc6n(-c6ccccc6)c5c4)ccc4oc5ccc6ccccc6c5c34)cc2)cc1 ; c1ccc(N(c2ccc3ccccc3c2)c2c(-c3ccc4c5ccc6ccccc6c5n(-c5ccccc5)c4c3)ccc3oc4ccc5ccccc5c4c23)cc1 ; c1ccc(-c2ccc(N(c3ccccc3)c3cc(-c4ccc5c6c7ccccc7ccc6n(-c6ccccc6)c5c4)cc4oc5ccc6ccccc6c5c34)cc2)cc1]

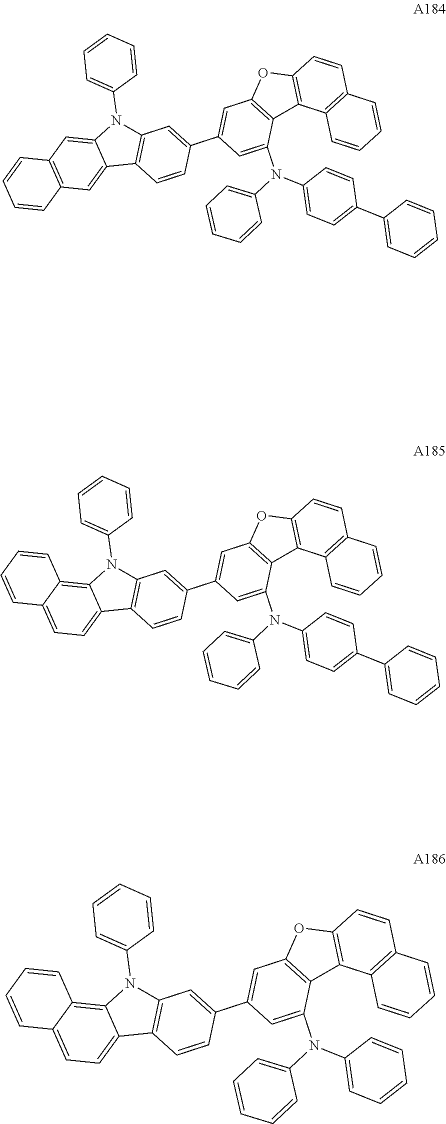 [SMILES: c1ccc(-c2ccc(N(c3ccccc3)c3cc(-c4ccc5c6cc7ccccc7cc6n(-c6ccccc6)c5c4)cc4oc5ccc6ccccc6c5c34)cc2)cc1 ; c1ccc(-c2ccc(N(c3ccccc3)c3cc(-c4ccc5c6ccc7ccccc7c6n(-c6ccccc6)c5c4)cc4oc5ccc6ccccc6c5c34)cc2)cc1 ; c1ccc(N(c2ccccc2)c2cc(-c3ccc4c5ccc6ccccc6c5n(-c5ccccc5)c4c3)cc3oc4ccc5ccccc5c4c23)cc1]

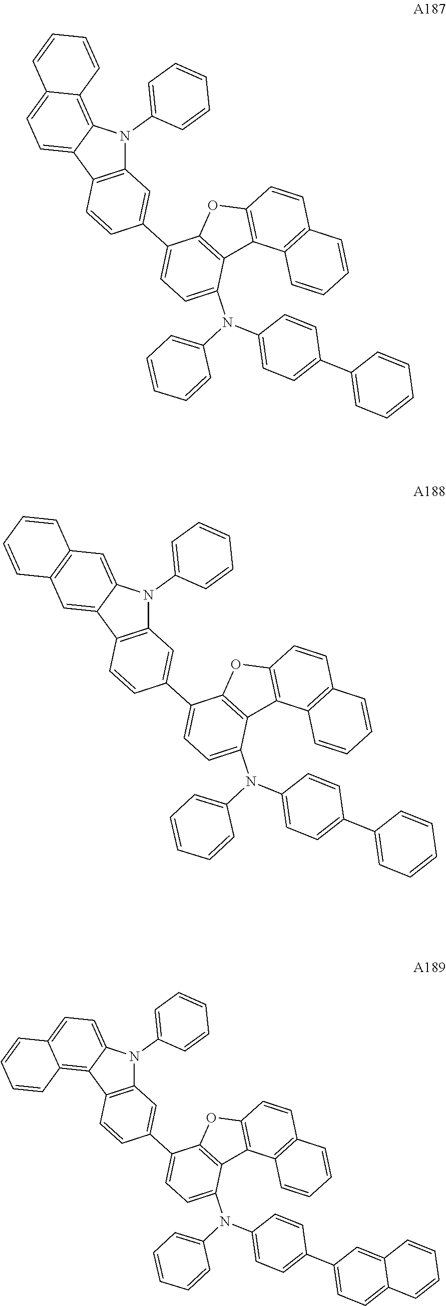 [SMILES: c1ccc(-c2ccc(N(c3ccccc3)c3ccc(-c4ccc5c6ccc7ccccc7c6n(-c6ccccc6)c5c4)c4oc5ccc6ccccc6c5c34)cc2)cc1 ; c1ccc(-c2ccc(N(c3ccccc3)c3ccc(-c4ccc5c6cc7ccccc7cc6n(-c6ccccc6)c5c4)c4oc5ccc6ccccc6c5c34)cc2)cc1 ; c1ccc(N(c2ccc(-c3ccc4ccccc4c3)cc2)c2ccc(-c3ccc4c5c6ccccc6ccc5n(-c5ccccc5)c4c3)c3oc4ccc5ccccc5c4c23)cc1]

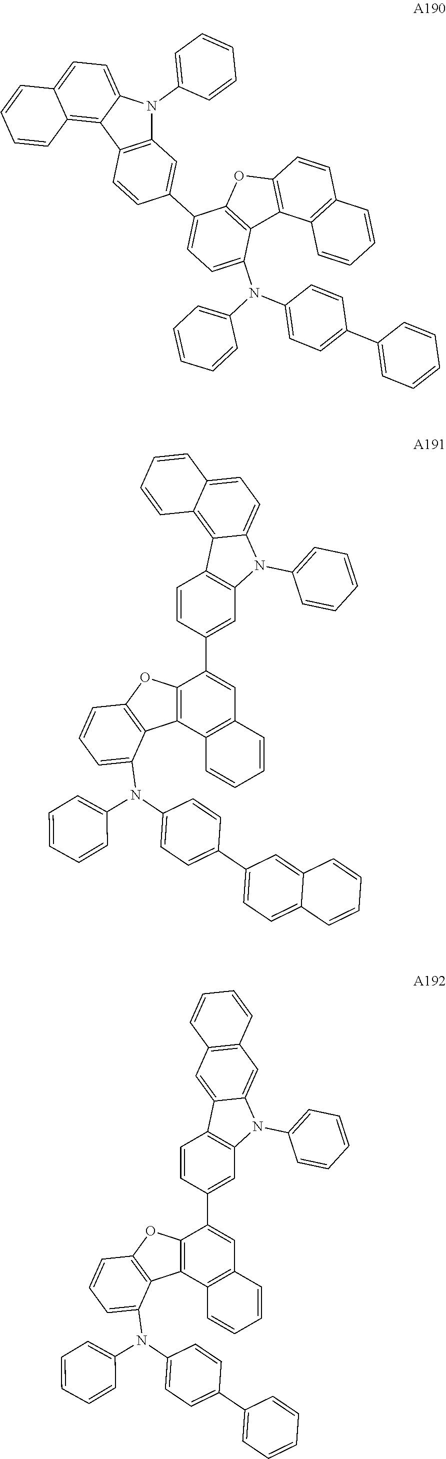 [SMILES: c1ccc(-c2ccc(N(c3ccccc3)c3ccc(-c4ccc5c6c7ccccc7ccc6n(-c6ccccc6)c5c4)c4oc5ccc6ccccc6c5c34)cc2)cc1 ; c1ccc(N(c2ccc(-c3ccc4ccccc4c3)cc2)c2cccc3oc4c(-c5ccc6c7c8ccccc8ccc7n(-c7ccccc7)c6c5)cc5ccccc5c4c23)cc1 ; c1ccc(-c2ccc(N(c3ccccc3)c3cccc4oc5c(-c6ccc7c8cc9ccccc9cc8n(-c8ccccc8)c7c6)cc6ccccc6c5c34)cc2)cc1]

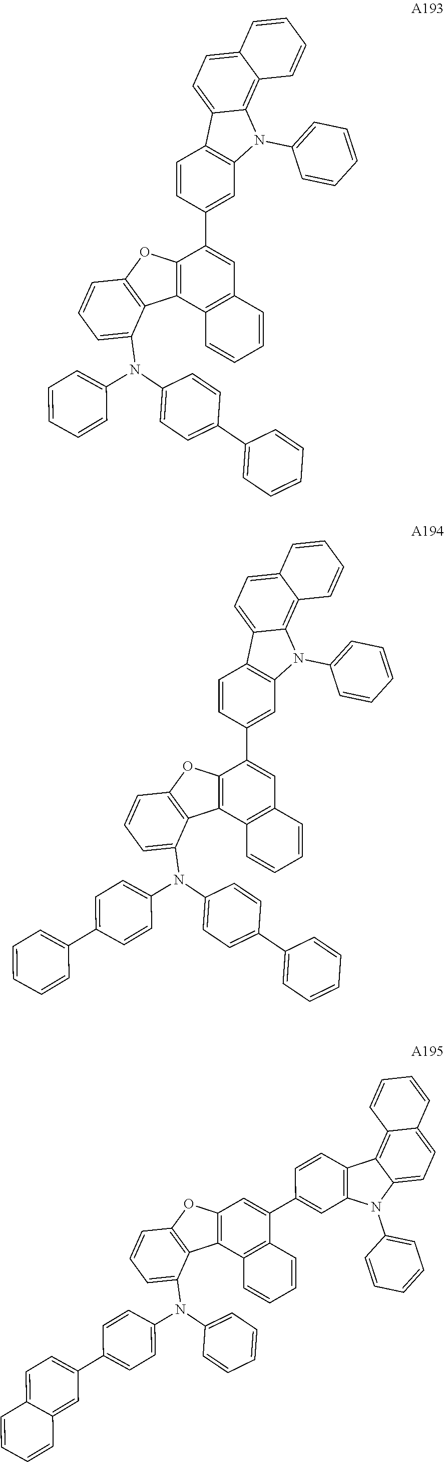 [SMILES: c1ccc(-c2ccc(N(c3ccccc3)c3cccc4oc5c(-c6ccc7c8ccc9ccccc9c8n(-c8ccccc8)c7c6)cc6ccccc6c5c34)cc2)cc1 ; c1ccc(-c2ccc(N(c3ccc(-c4ccccc4)cc3)c3cccc4oc5c(-c6ccc7c8ccc9ccccc9c8n(-c8ccccc8)c7c6)cc6ccccc6c5c34)cc2)cc1 ; c1ccc(N(c2ccc(-c3ccc4ccccc4c3)cc2)c2cccc3oc4cc(-c5ccc6c7c8ccccc8ccc7n(-c7ccccc7)c6c5)c5ccccc5c4c23)cc1]

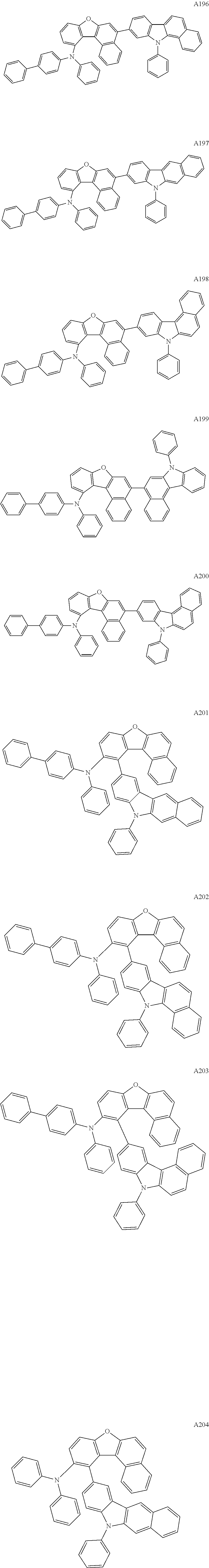

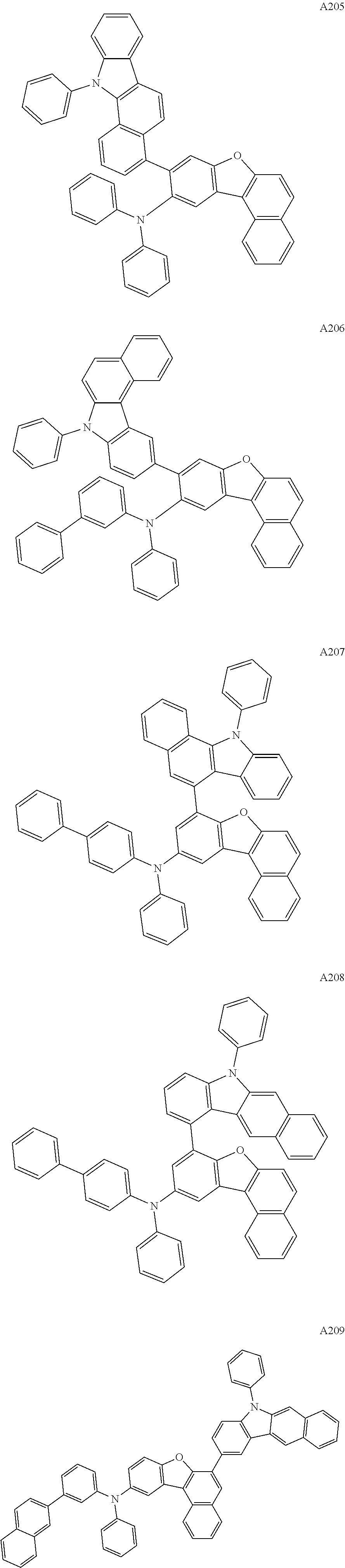 [SMILES: c1ccc(N(c2ccccc2)c2cc3c(cc2-c2cccc4c2ccc2c5ccccc5n(-c5ccccc5)c42)oc2ccc4ccccc4c23)cc1 ; c1ccc(-c2cccc(N(c3ccccc3)c3cc4c(cc3-c3ccc5c(c3)c3c6ccccc6ccc3n5-c3ccccc3)oc3ccc5ccccc5c34)c2)cc1 ; c1ccc(-c2ccc(N(c3ccccc3)c3cc(-c4cc5ccccc5c5c4c4ccccc4n5-c4ccccc4)c4oc5ccc6ccccc6c5c4c3)cc2)cc1 ; c1ccc(-c2ccc(N(c3ccccc3)c3cc(-c4cccc5c4c4cc6ccccc6cc4n5-c4ccccc4)c4oc5ccc6ccccc6c5c4c3)cc2)cc1 ; c1ccc(N(c2cccc(-c3ccc4ccccc4c3)c2)c2ccc3oc4c(-c5ccc6c(c5)c5cc7ccccc7cc5n6-c5ccccc5)cc5ccccc5c4c3c2)cc1]

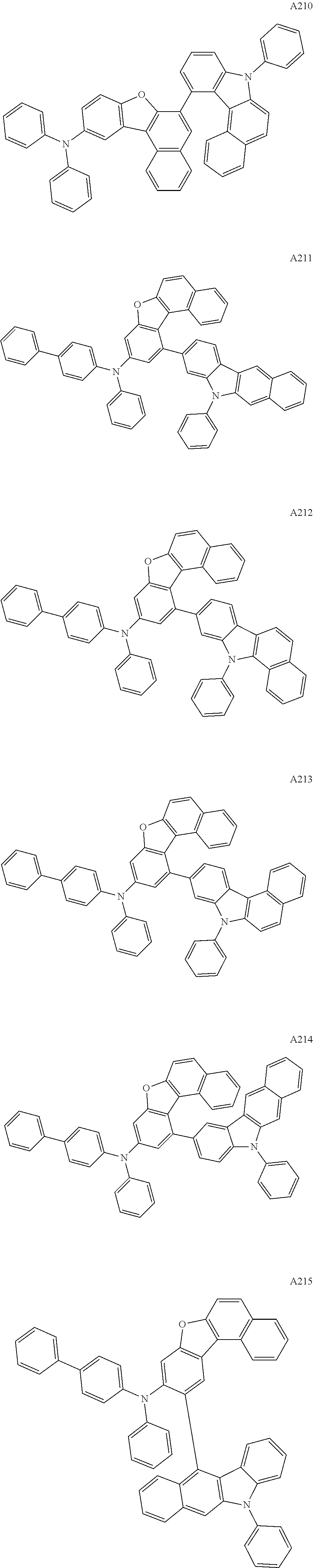

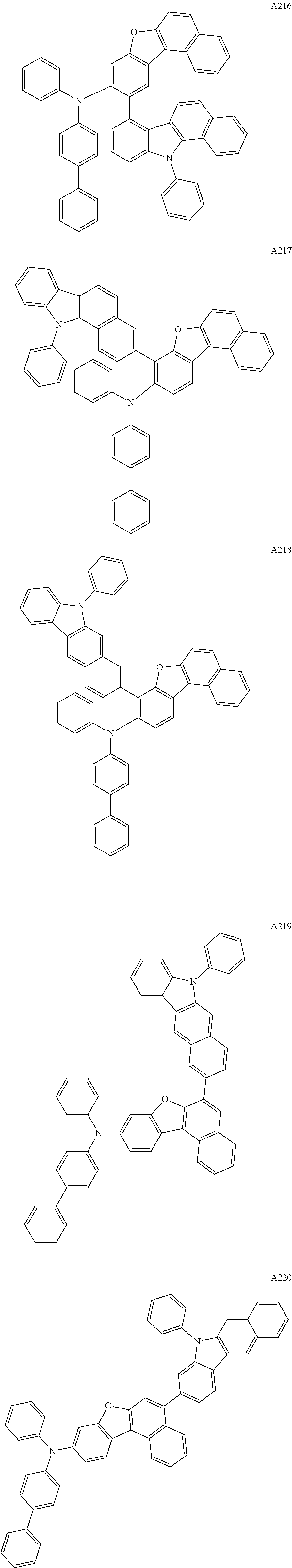

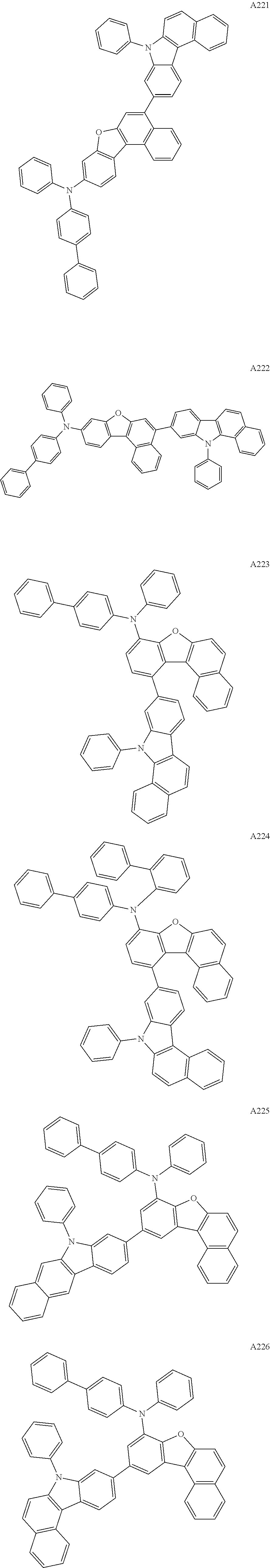

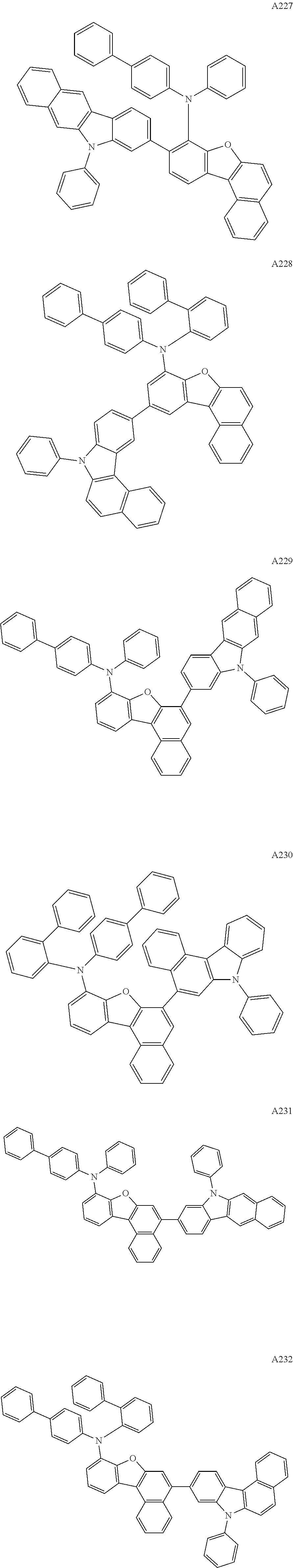

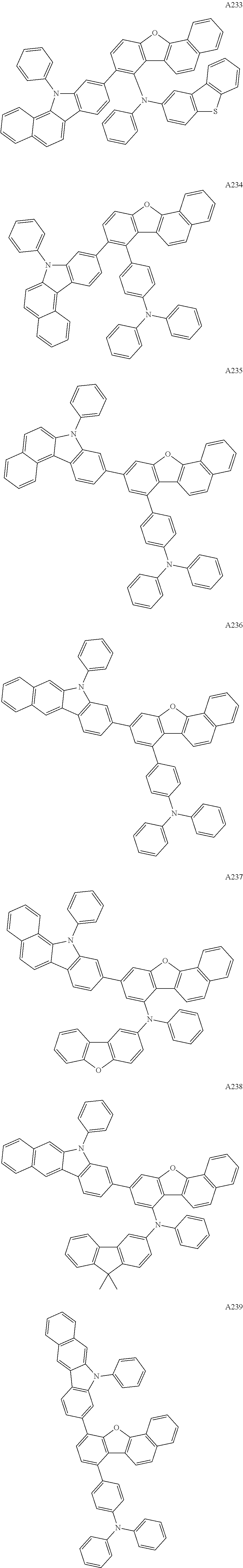

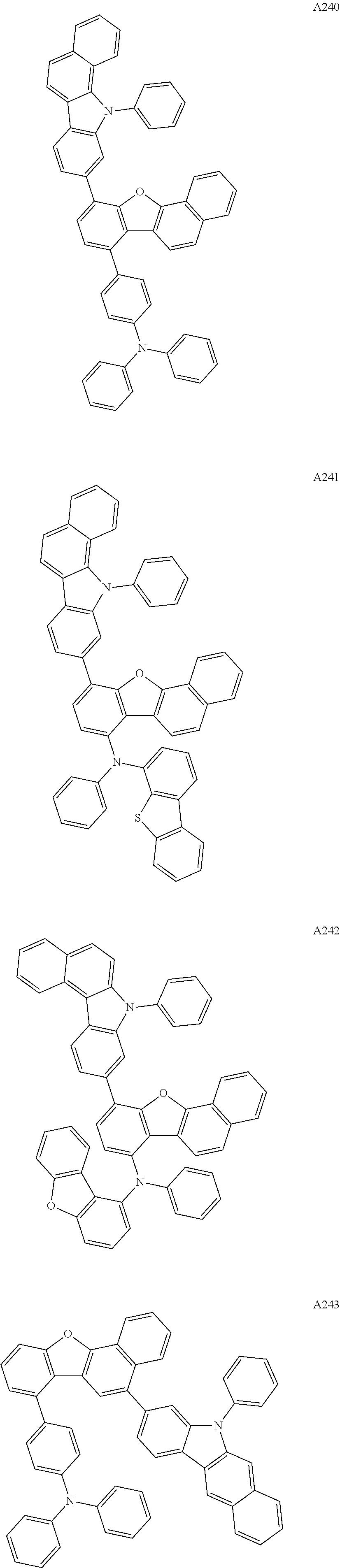 [SMILES: c1ccc(N(c2ccccc2)c2ccc(-c3ccc(-c4ccc5c6ccc7ccccc7c6n(-c6ccccc6)c5c4)c4oc5c6ccccc6ccc5c34)cc2)cc1 ; c1ccc(N(c2cccc3c2sc2ccccc23)c2ccc(-c3ccc4c5ccc6ccccc6c5n(-c5ccccc5)c4c3)c3oc4c5ccccc5ccc4c23)cc1 ; c1ccc(N(c2cccc3oc4ccccc4c23)c2ccc(-c3ccc4c5c6ccccc6ccc5n(-c5ccccc5)c4c3)c3oc4c5ccccc5ccc4c23)cc1 ; c1ccc(N(c2ccccc2)c2ccc(-c3cccc4oc5c6ccccc6c(-c6ccc7c8cc9ccccc9cc8n(-c8ccccc8)c7c6)cc5c34)cc2)cc1]

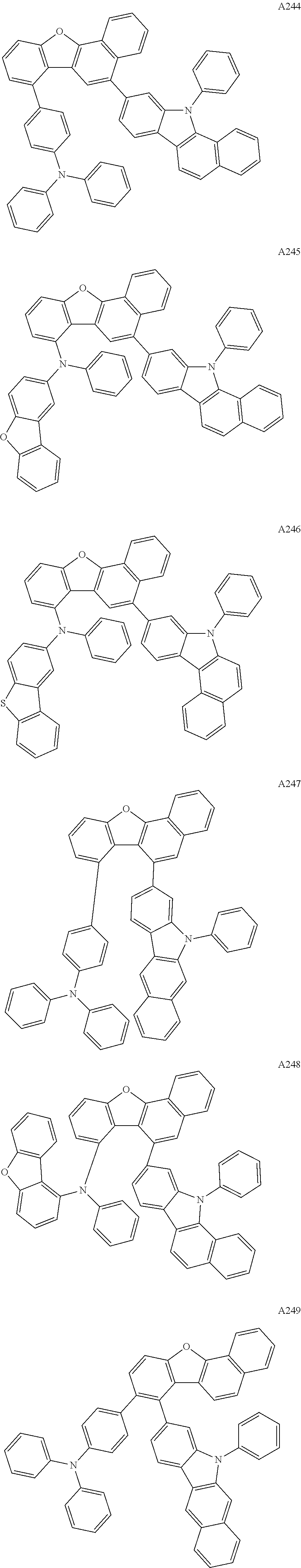

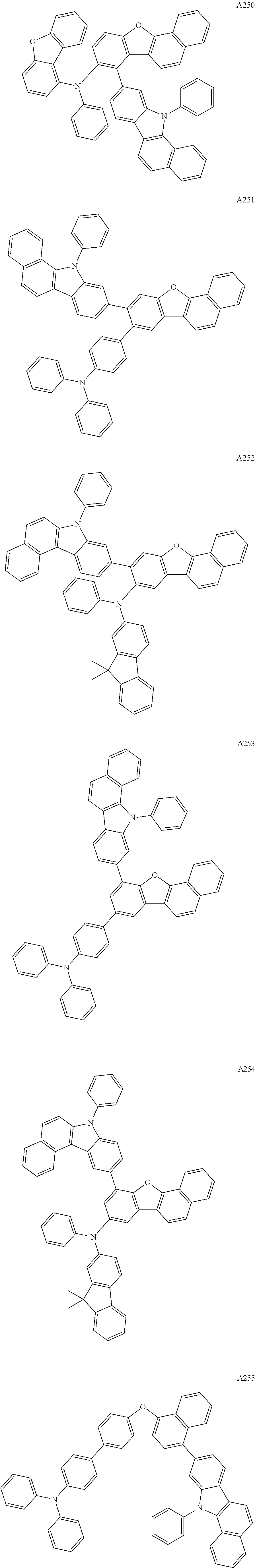

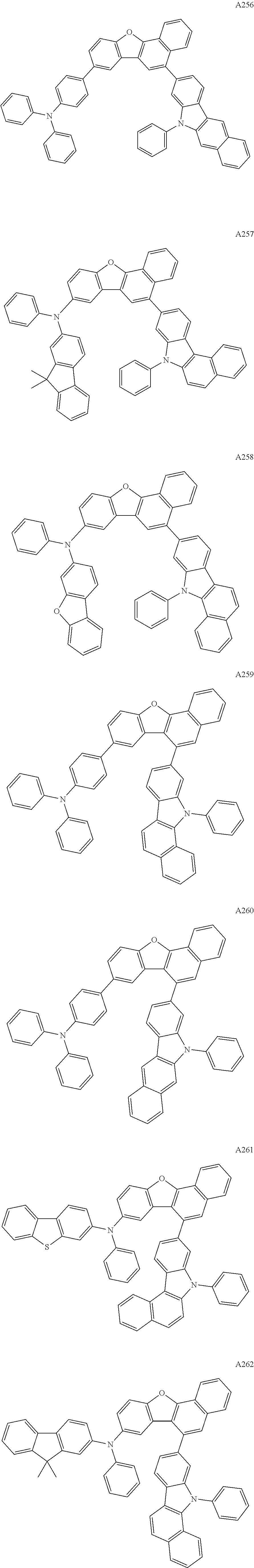

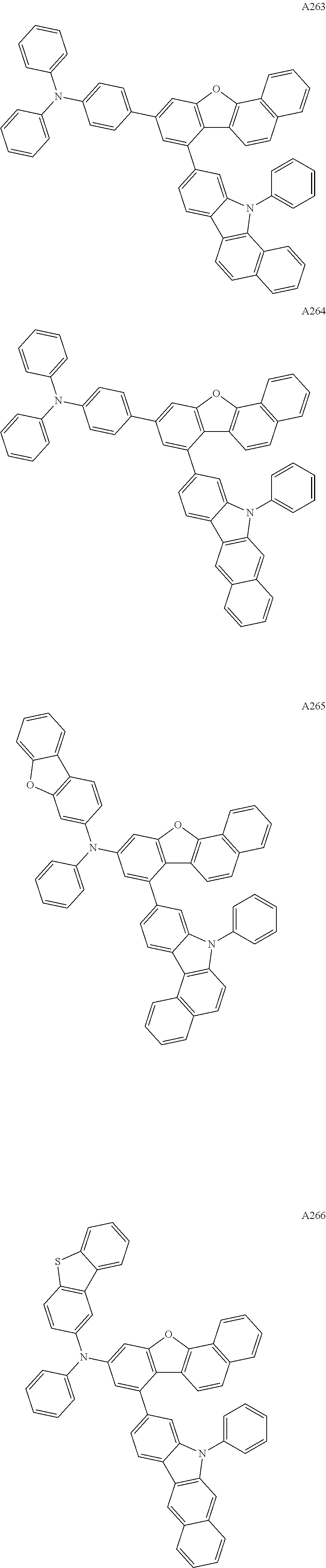 [SMILES: c1ccc(N(c2ccccc2)c2ccc(-c3cc(-c4ccc5c6ccc7ccccc7c6n(-c6ccccc6)c5c4)c4c(c3)oc3c5ccccc5ccc34)cc2)cc1 ; c1ccc(N(c2ccccc2)c2ccc(-c3cc(-c4ccc5c6cc7ccccc7cc6n(-c6ccccc6)c5c4)c4c(c3)oc3c5ccccc5ccc34)cc2)cc1 ; c1ccc(N(c2ccc3c(c2)oc2ccccc23)c2cc(-c3ccc4c5c6ccccc6ccc5n(-c5ccccc5)c4c3)c3c(c2)oc2c4ccccc4ccc23)cc1 ; c1ccc(N(c2cc(-c3ccc4c5cc6ccccc6cc5n(-c5ccccc5)c4c3)c3c(c2)oc2c4ccccc4ccc23)c2ccc3sc4ccccc4c3c2)cc1]

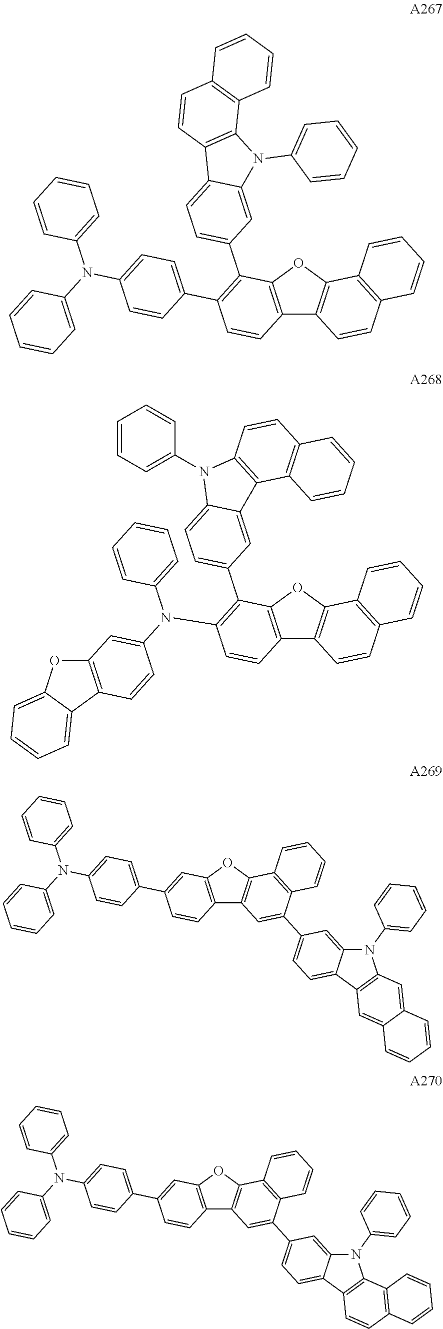

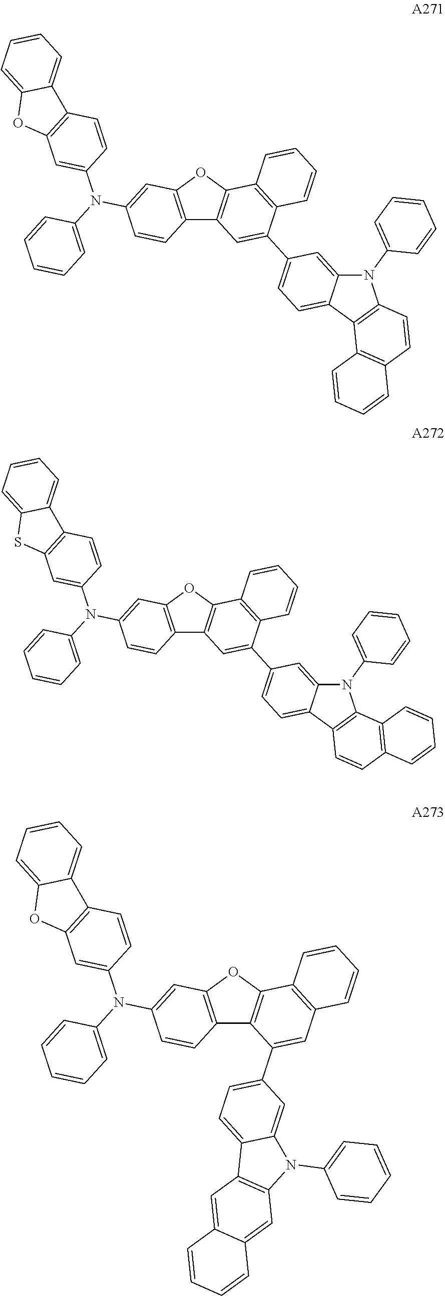 [SMILES: c1ccc(N(c2ccc3c(c2)oc2ccccc23)c2ccc3c(c2)oc2c4ccccc4c(-c4ccc5c6c7ccccc7ccc6n(-c6ccccc6)c5c4)cc32)cc1 ; c1ccc(N(c2ccc3c(c2)oc2c4ccccc4c(-c4ccc5c6ccc7ccccc7c6n(-c6ccccc6)c5c4)cc32)c2ccc3c(c2)sc2ccccc23)cc1 ; c1ccc(N(c2ccc3c(c2)oc2ccccc23)c2ccc3c(c2)oc2c4ccccc4cc(-c4ccc5c6cc7ccccc7cc6n(-c6ccccc6)c5c4)c32)cc1]

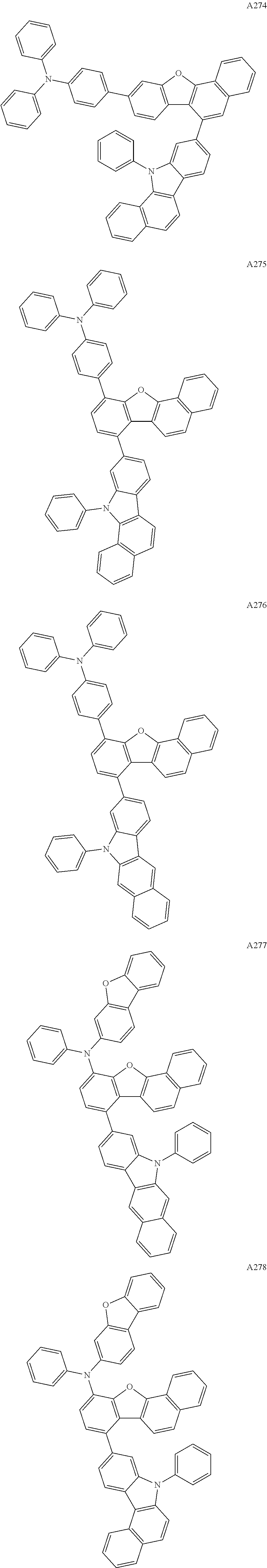

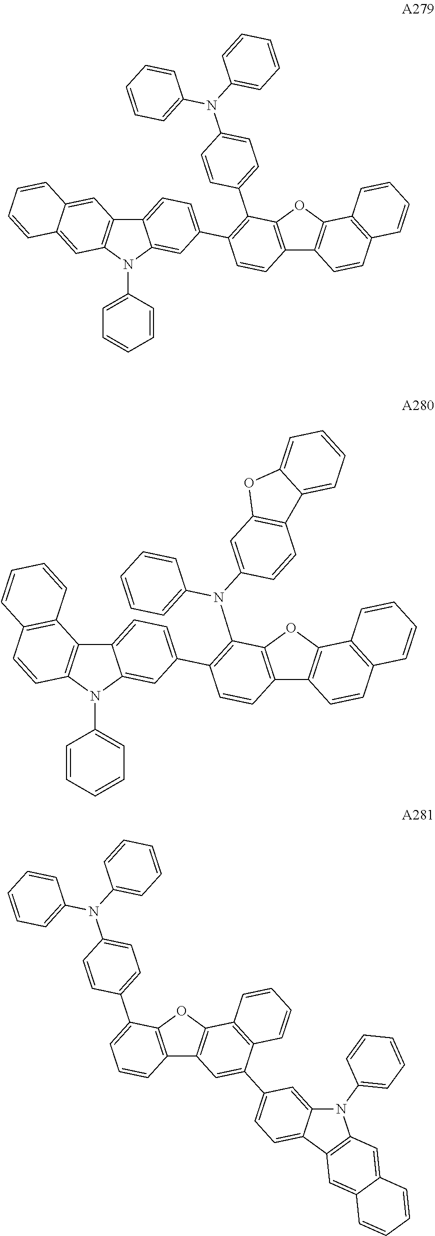 [SMILES: c1ccc(N(c2ccccc2)c2ccc(-c3c(-c4ccc5c6cc7ccccc7cc6n(-c6ccccc6)c5c4)ccc4c3oc3c5ccccc5ccc43)cc2)cc1 ; c1ccc(N(c2ccc3c(c2)oc2ccccc23)c2c(-c3ccc4c5c6ccccc6ccc5n(-c5ccccc5)c4c3)ccc3c2oc2c4ccccc4ccc32)cc1 ; c1ccc(N(c2ccccc2)c2ccc(-c3cccc4c3oc3c5ccccc5c(-c5ccc6c7cc8ccccc8cc7n(-c7ccccc7)c6c5)cc43)cc2)cc1]

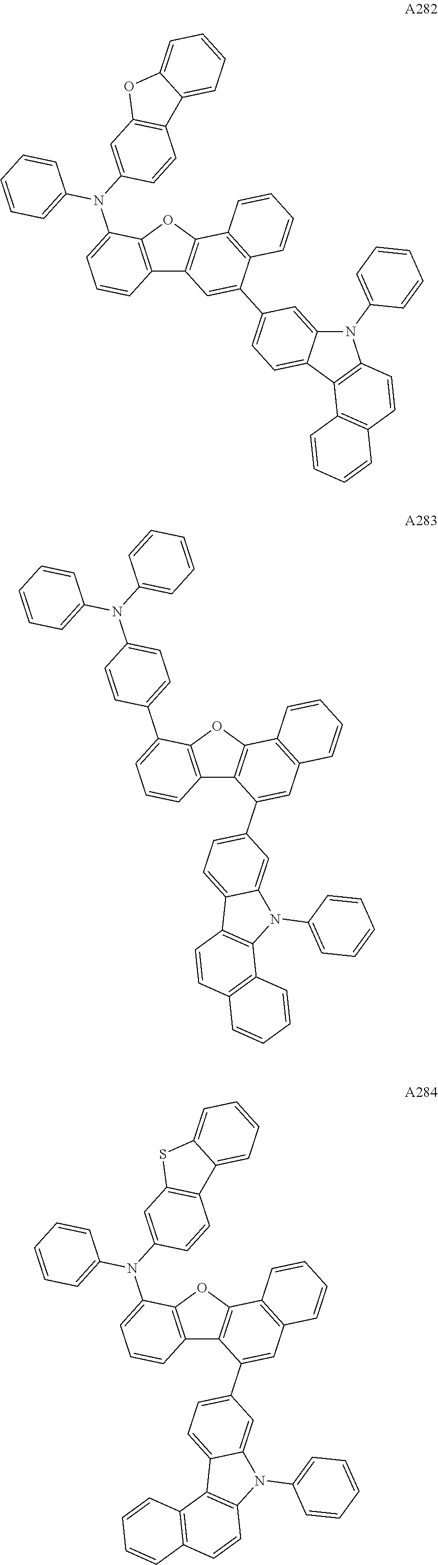 [SMILES: c1ccc(N(c2ccc3c(c2)oc2ccccc23)c2cccc3c2oc2c4ccccc4c(-c4ccc5c6c7ccccc7ccc6n(-c6ccccc6)c5c4)cc32)cc1 ; c1ccc(N(c2ccccc2)c2ccc(-c3cccc4c3oc3c5ccccc5cc(-c5ccc6c7ccc8ccccc8c7n(-c7ccccc7)c6c5)c43)cc2)cc1 ; c1ccc(N(c2ccc3c(c2)sc2ccccc23)c2cccc3c2oc2c4ccccc4cc(-c4ccc5c6c7ccccc7ccc6n(-c6ccccc6)c5c4)c32)cc1]

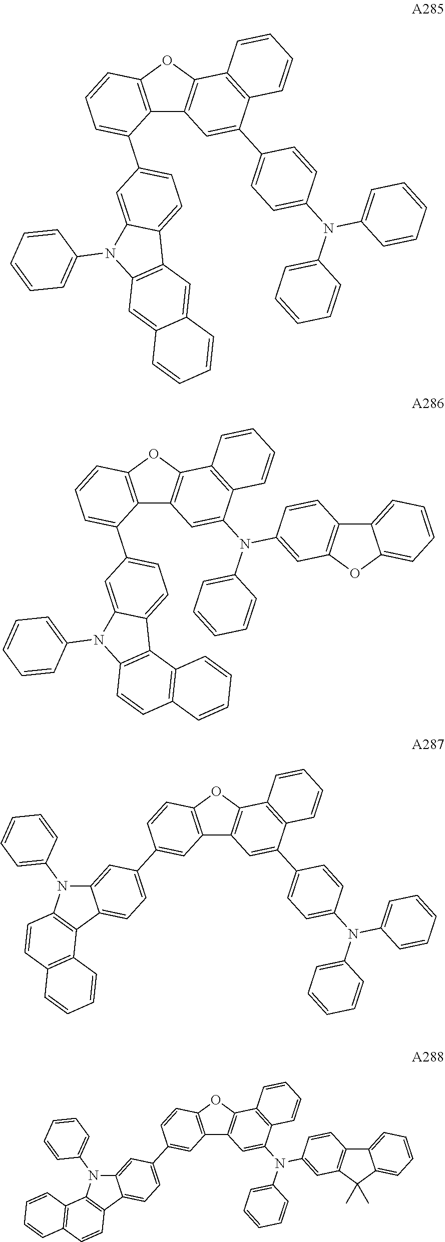 [SMILES: c1ccc(N(c2ccccc2)c2ccc(-c3cc4c(oc5cccc(-c6ccc7c8cc9ccccc9cc8n(-c8ccccc8)c7c6)c54)c4ccccc34)cc2)cc1 ; c1ccc(N(c2ccc3c(c2)oc2ccccc23)c2cc3c(oc4cccc(-c5ccc6c7c8ccccc8ccc7n(-c7ccccc7)c6c5)c43)c3ccccc23)cc1 ; c1ccc(N(c2ccccc2)c2ccc(-c3cc4c5cc(-c6ccc7c8c9ccccc9ccc8n(-c8ccccc8)c7c6)ccc5oc4c4ccccc34)cc2)cc1 ; CC1(C)c2ccccc2-c2ccc(N(c3ccccc3)c3cc4c5cc(-c6ccc7c8ccc9ccccc9c8n(-c8ccccc8)c7c6)ccc5oc4c4ccccc34)cc21]

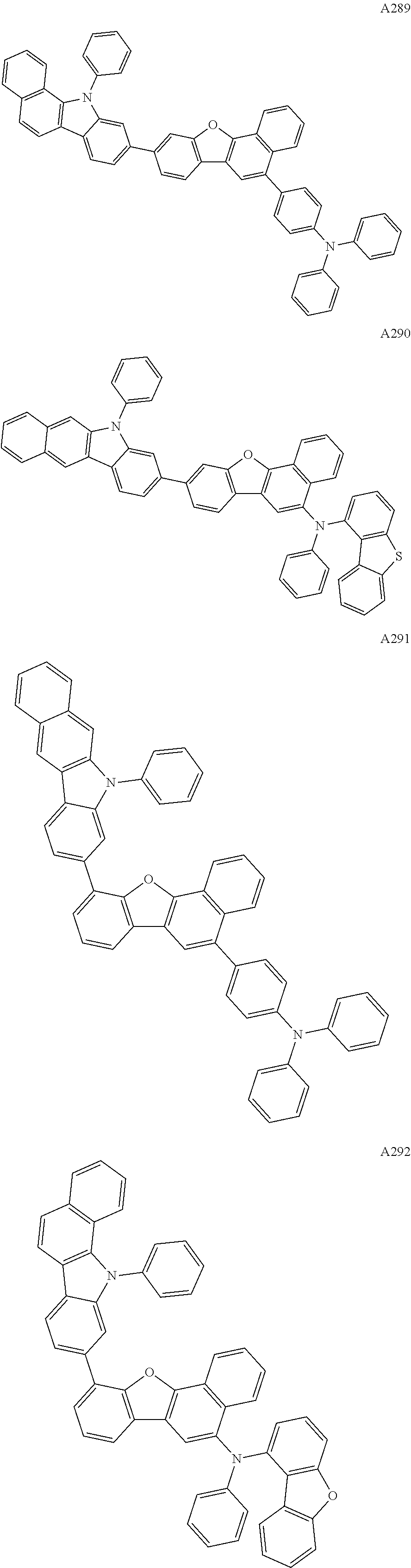 [SMILES: c1ccc(N(c2ccccc2)c2ccc(-c3cc4c5ccc(-c6ccc7c8ccc9ccccc9c8n(-c8ccccc8)c7c6)cc5oc4c4ccccc34)cc2)cc1 ; c1ccc(N(c2cc3c4ccc(-c5ccc6c7cc8ccccc8cc7n(-c7ccccc7)c6c5)cc4oc3c3ccccc23)c2cccc3sc4ccccc4c23)cc1 ; c1ccc(N(c2ccccc2)c2ccc(-c3cc4c5cccc(-c6ccc7c8cc9ccccc9cc8n(-c8ccccc8)c7c6)c5oc4c4ccccc34)cc2)cc1 ; c1ccc(N(c2cc3c4cccc(-c5ccc6c7ccc8ccccc8c7n(-c7ccccc7)c6c5)c4oc3c3ccccc23)c2cccc3oc4ccccc4c23)cc1]

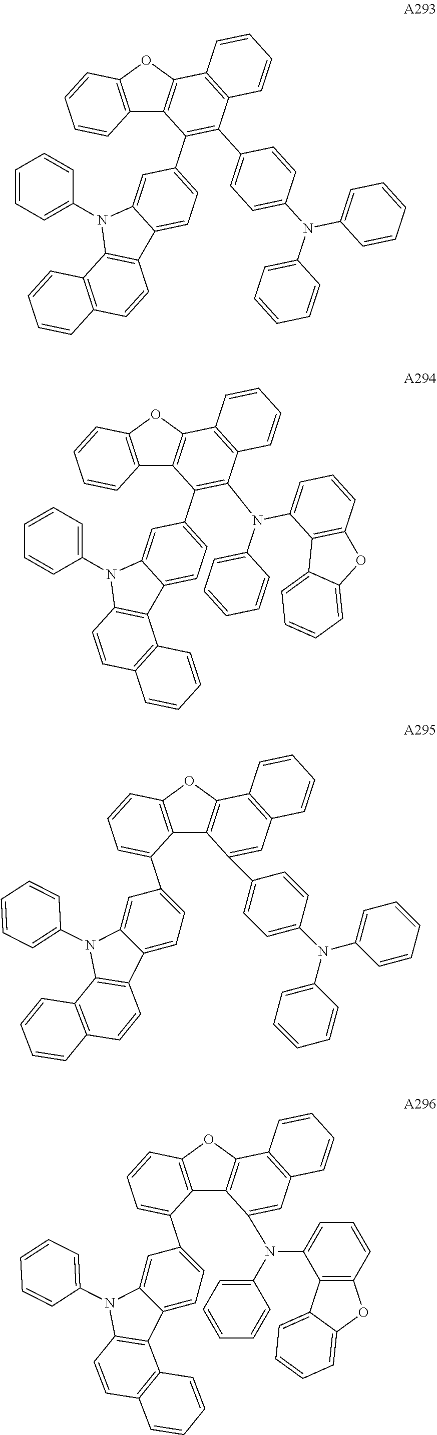 [SMILES: c1ccc(N(c2ccccc2)c2ccc(-c3c(-c4ccc5c6ccc7ccccc7c6n(-c6ccccc6)c5c4)c4c5ccccc5oc4c4ccccc34)cc2)cc1 ; c1ccc(N(c2c(-c3ccc4c5c6ccccc6ccc5n(-c5ccccc5)c4c3)c3c4ccccc4oc3c3ccccc23)c2cccc3oc4ccccc4c23)cc1 ; c1ccc(N(c2ccccc2)c2ccc(-c3cc4ccccc4c4oc5cccc(-c6ccc7c8ccc9ccccc9c8n(-c8ccccc8)c7c6)c5c34)cc2)cc1 ; c1ccc(N(c2cccc3oc4ccccc4c23)c2cc3ccccc3c3oc4cccc(-c5ccc6c7c8ccccc8ccc7n(-c7ccccc7)c6c5)c4c23)cc1]

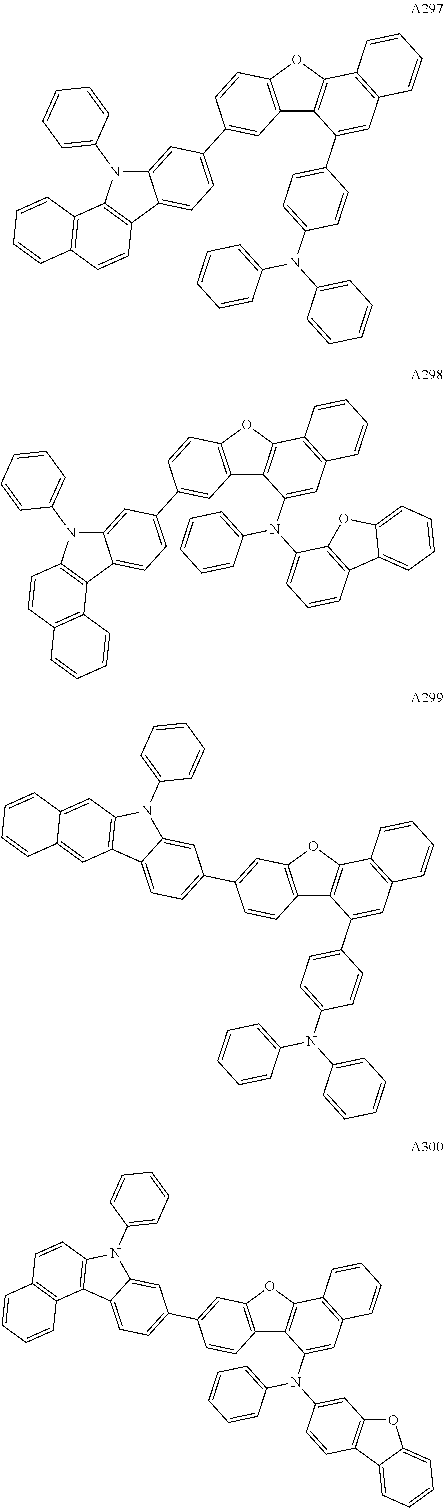 [SMILES: c1ccc(N(c2ccccc2)c2ccc(-c3cc4ccccc4c4oc5ccc(-c6ccc7c8ccc9ccccc9c8n(-c8ccccc8)c7c6)cc5c34)cc2)cc1 ; c1ccc(N(c2cccc3c2oc2ccccc23)c2cc3ccccc3c3oc4ccc(-c5ccc6c7c8ccccc8ccc7n(-c7ccccc7)c6c5)cc4c23)cc1 ; c1ccc(N(c2ccccc2)c2ccc(-c3cc4ccccc4c4oc5cc(-c6ccc7c8cc9ccccc9cc8n(-c8ccccc8)c7c6)ccc5c34)cc2)cc1 ; c1ccc(N(c2ccc3c(c2)oc2ccccc23)c2cc3ccccc3c3oc4cc(-c5ccc6c7c8ccccc8ccc7n(-c7ccccc7)c6c5)ccc4c23)cc1]

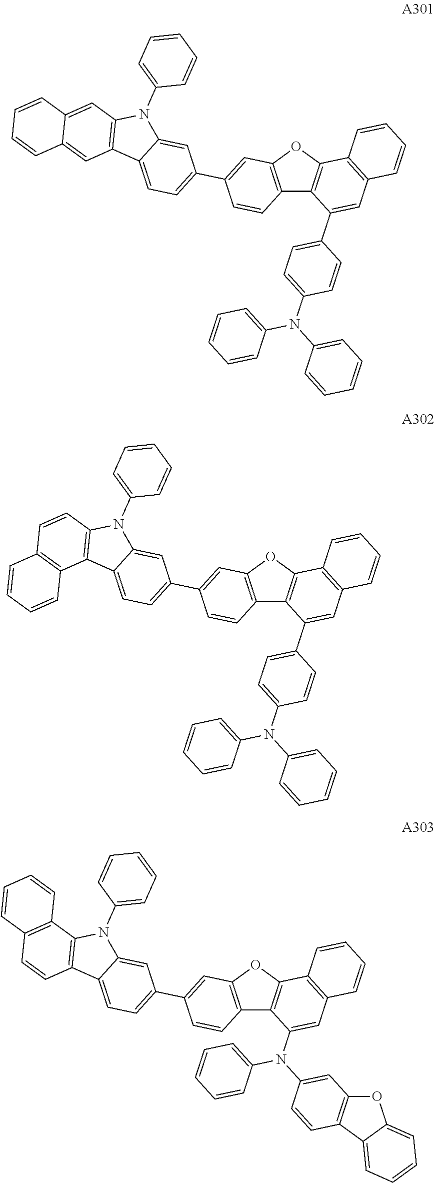 [SMILES: c1ccc(N(c2ccccc2)c2ccc(-c3cc4ccccc4c4oc5cc(-c6ccc7c8cc9ccccc9cc8n(-c8ccccc8)c7c6)ccc5c34)cc2)cc1 ; c1ccc(N(c2ccccc2)c2ccc(-c3cc4ccccc4c4oc5cc(-c6ccc7c8c9ccccc9ccc8n(-c8ccccc8)c7c6)ccc5c34)cc2)cc1 ; c1ccc(N(c2ccc3c(c2)oc2ccccc23)c2cc3ccccc3c3oc4cc(-c5ccc6c7ccc8ccccc8c7n(-c7ccccc7)c6c5)ccc4c23)cc1]

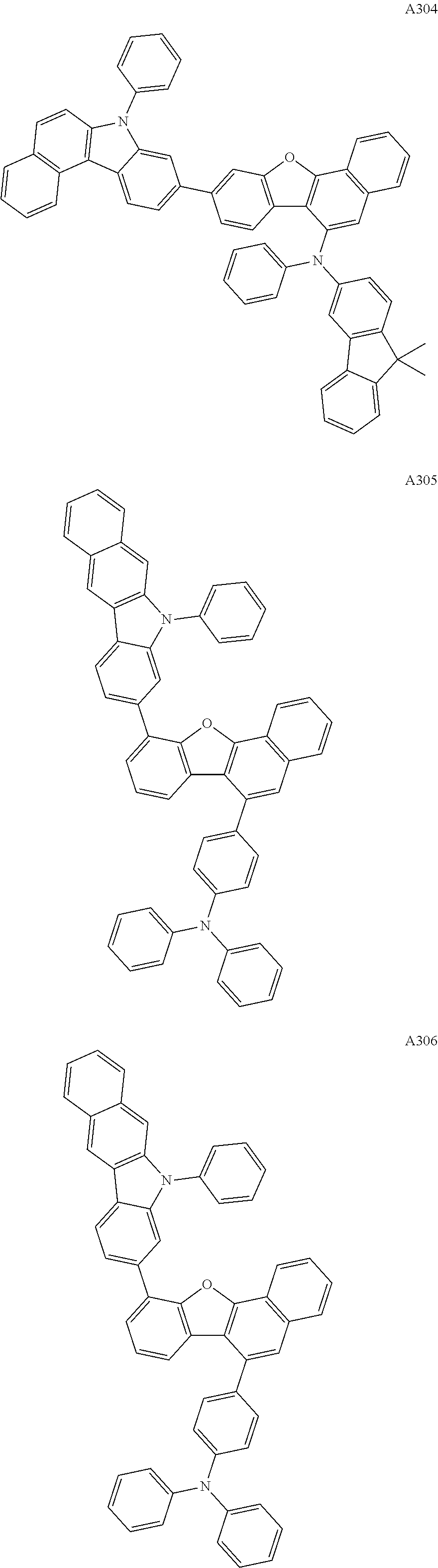 [SMILES: CC1(C)c2ccccc2-c2cc(N(c3ccccc3)c3cc4ccccc4c4oc5cc(-c6ccc7c8c9ccccc9ccc8n(-c8ccccc8)c7c6)ccc5c34)ccc21 ; c1ccc(N(c2ccccc2)c2ccc(-c3cc4ccccc4c4oc5c(-c6ccc7c8cc9ccccc9cc8n(-c8ccccc8)c7c6)cccc5c34)cc2)cc1 ; c1ccc(N(c2ccccc2)c2ccc(-c3cc4ccccc4c4oc5c(-c6ccc7c8cc9ccccc9cc8n(-c8ccccc8)c7c6)cccc5c34)cc2)cc1]

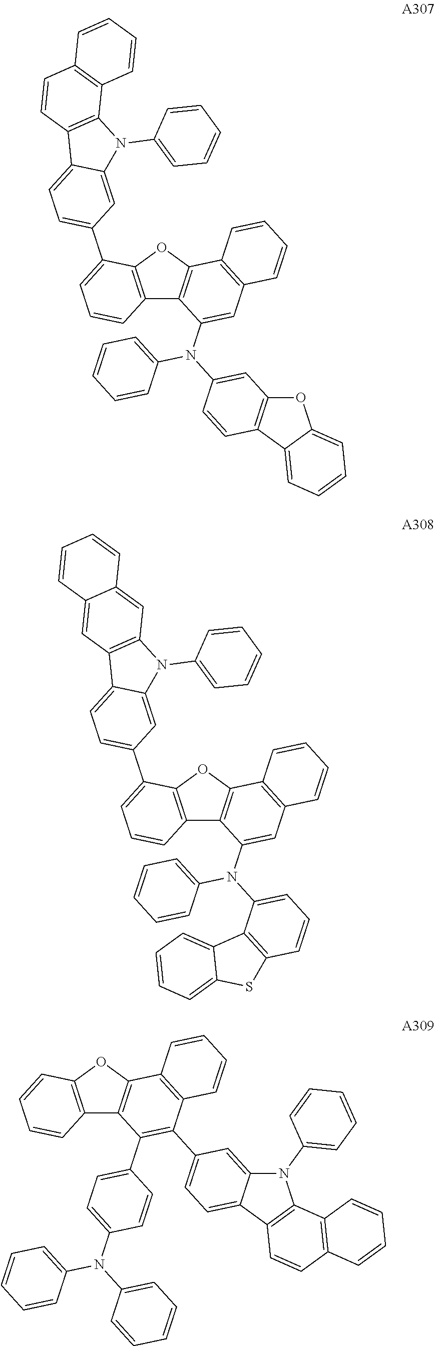

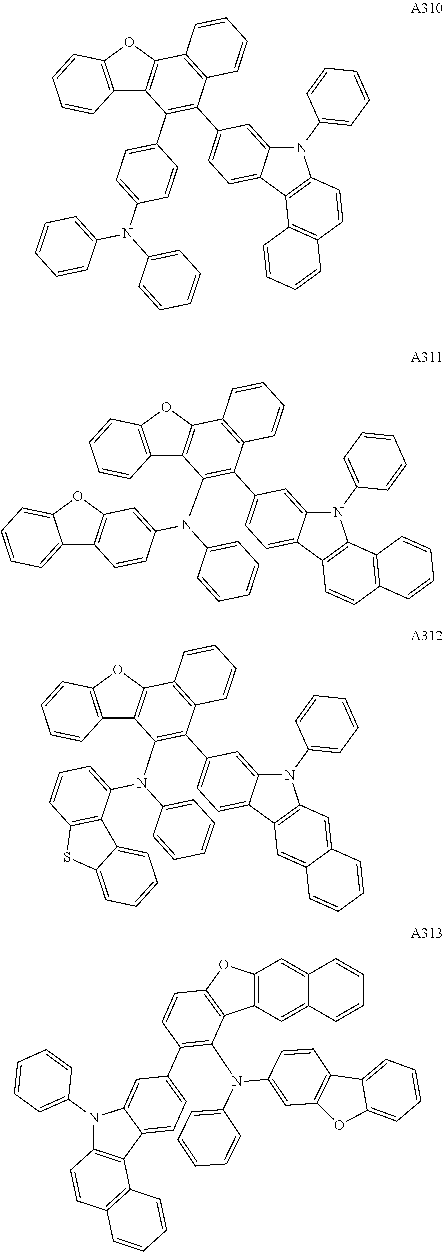 [SMILES: c1ccc(N(c2ccccc2)c2ccc(-c3c(-c4ccc5c6c7ccccc7ccc6n(-c6ccccc6)c5c4)c4ccccc4c4oc5ccccc5c34)cc2)cc1 ; c1ccc(N(c2ccc3c(c2)oc2ccccc23)c2c(-c3ccc4c5ccc6ccccc6c5n(-c5ccccc5)c4c3)c3ccccc3c3oc4ccccc4c23)cc1 ; c1ccc(N(c2cccc3sc4ccccc4c23)c2c(-c3ccc4c5cc6ccccc6cc5n(-c5ccccc5)c4c3)c3ccccc3c3oc4ccccc4c23)cc1 ; c1ccc(N(c2ccc3c(c2)oc2ccccc23)c2c(-c3ccc4c5c6ccccc6ccc5n(-c5ccccc5)c4c3)ccc3oc4cc5ccccc5cc4c23)cc1]

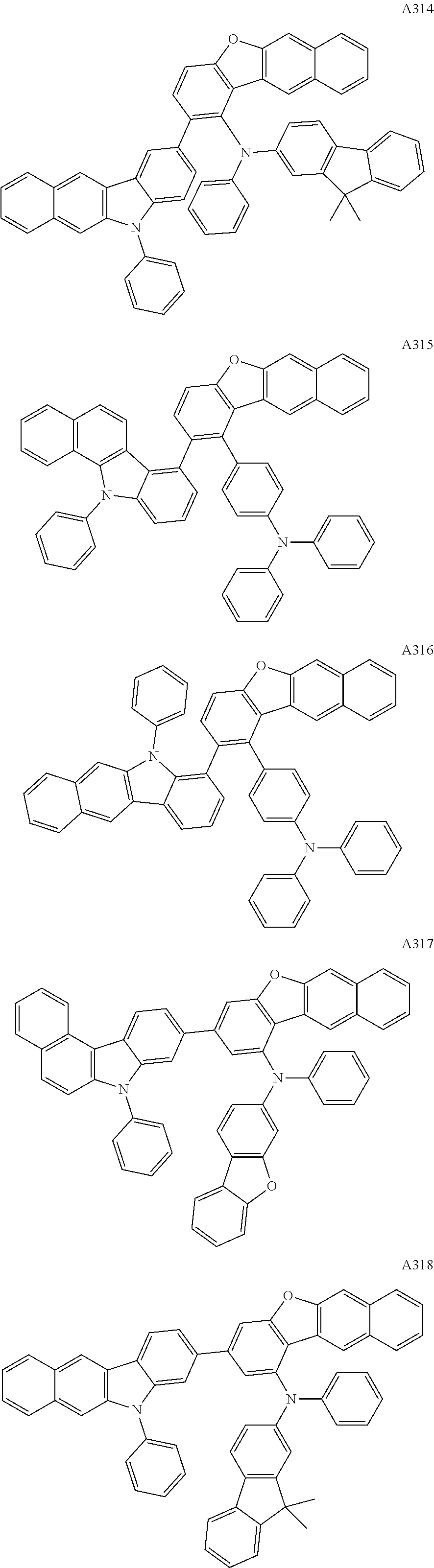

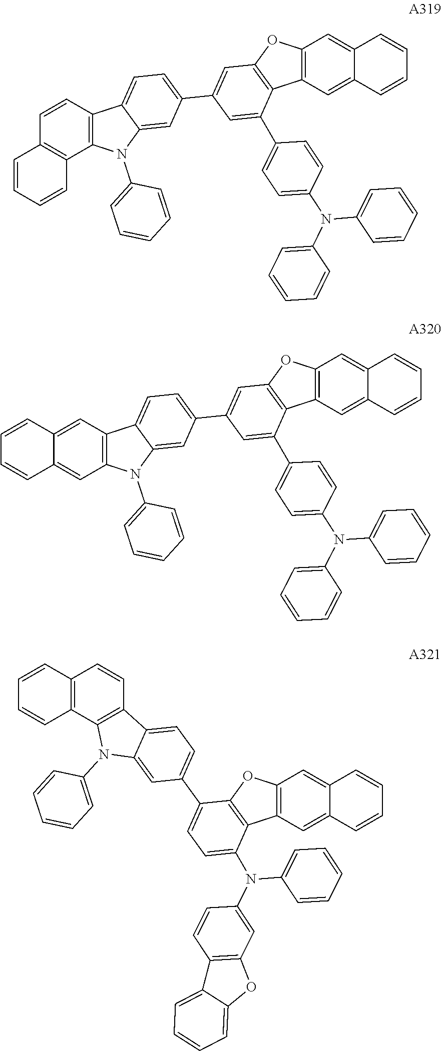 [SMILES: c1ccc(N(c2ccccc2)c2ccc(-c3cc(-c4ccc5c6ccc7ccccc7c6n(-c6ccccc6)c5c4)cc4oc5cc6ccccc6cc5c34)cc2)cc1 ; c1ccc(N(c2ccccc2)c2ccc(-c3cc(-c4ccc5c6cc7ccccc7cc6n(-c6ccccc6)c5c4)cc4oc5cc6ccccc6cc5c34)cc2)cc1 ; c1ccc(N(c2ccc3c(c2)oc2ccccc23)c2ccc(-c3ccc4c5ccc6ccccc6c5n(-c5ccccc5)c4c3)c3oc4cc5ccccc5cc4c23)cc1]

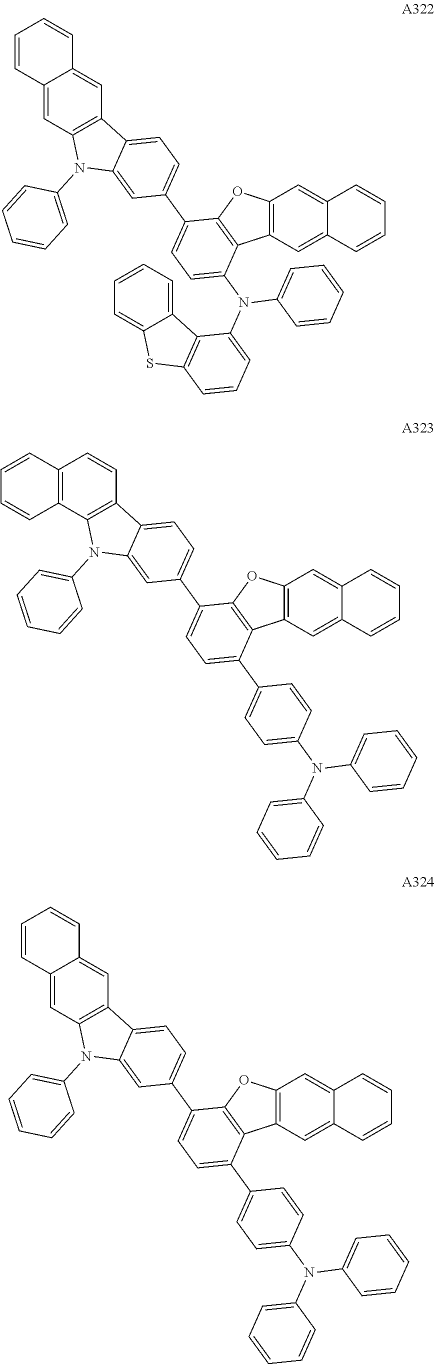 [SMILES: c1ccc(N(c2cccc3sc4ccccc4c23)c2ccc(-c3ccc4c5cc6ccccc6cc5n(-c5ccccc5)c4c3)c3oc4cc5ccccc5cc4c23)cc1 ; c1ccc(N(c2ccccc2)c2ccc(-c3ccc(-c4ccc5c6ccc7ccccc7c6n(-c6ccccc6)c5c4)c4oc5cc6ccccc6cc5c34)cc2)cc1 ; c1ccc(N(c2ccccc2)c2ccc(-c3ccc(-c4ccc5c6cc7ccccc7cc6n(-c6ccccc6)c5c4)c4oc5cc6ccccc6cc5c34)cc2)cc1]

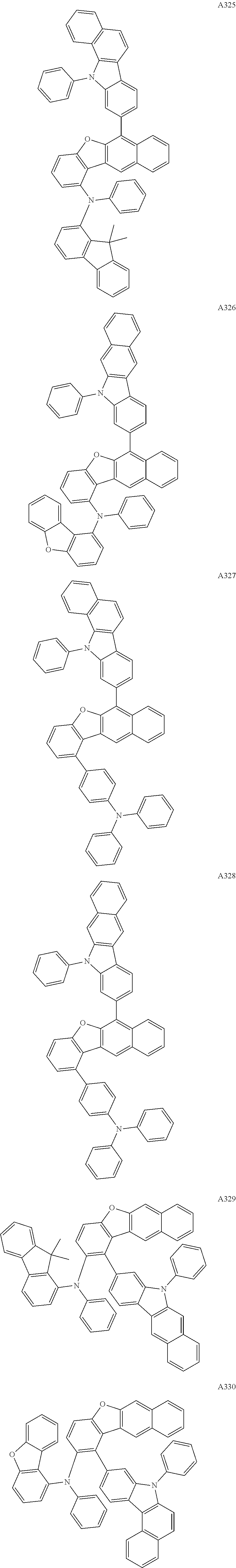

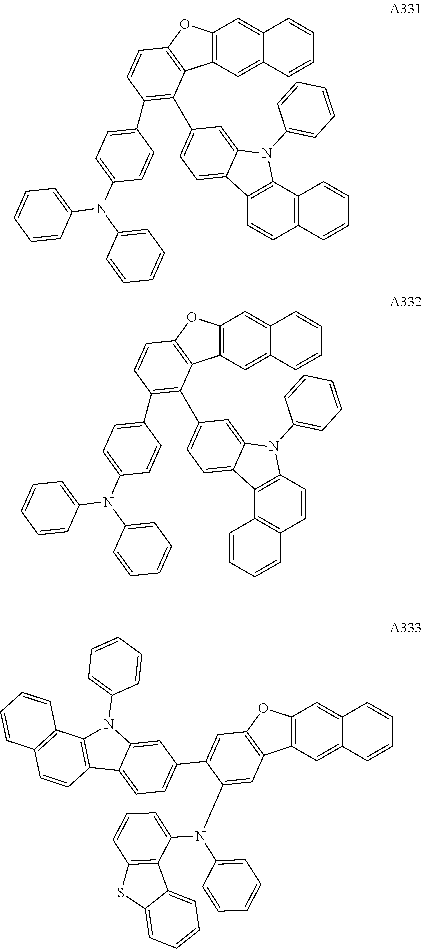 [SMILES: c1ccc(N(c2ccccc2)c2ccc(-c3ccc4oc5cc6ccccc6cc5c4c3-c3ccc4c5ccc6ccccc6c5n(-c5ccccc5)c4c3)cc2)cc1 ; c1ccc(N(c2ccccc2)c2ccc(-c3ccc4oc5cc6ccccc6cc5c4c3-c3ccc4c5c6ccccc6ccc5n(-c5ccccc5)c4c3)cc2)cc1 ; c1ccc(N(c2cc3c(cc2-c2ccc4c5ccc6ccccc6c5n(-c5ccccc5)c4c2)oc2cc4ccccc4cc23)c2cccc3sc4ccccc4c23)cc1]

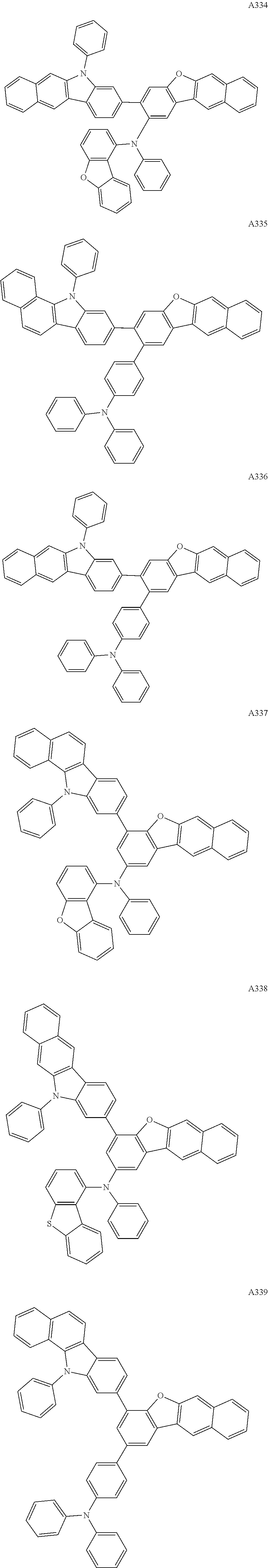

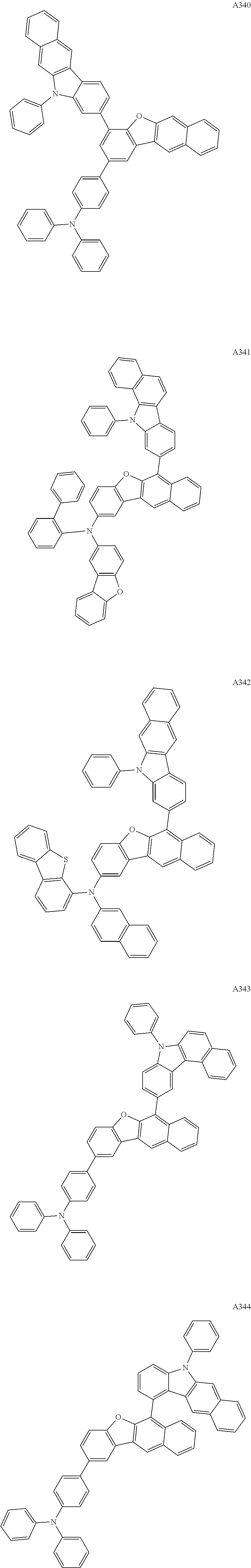

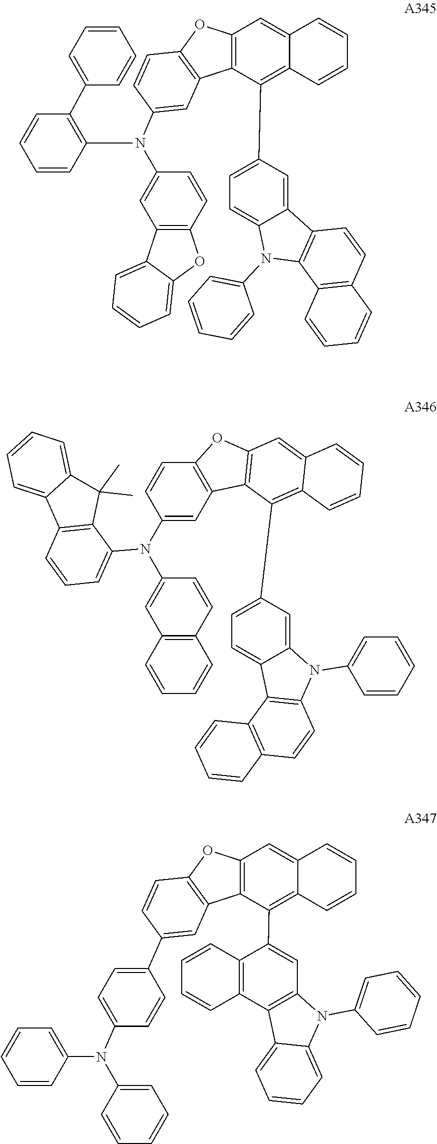 [SMILES: c1ccc(-c2ccccc2N(c2ccc3oc4ccccc4c3c2)c2ccc3oc4cc5ccccc5c(-c5ccc6c(c5)c5ccc7ccccc7c5n6-c5ccccc5)c4c3c2)cc1 ; CC1(C)c2ccccc2-c2cccc(N(c3ccc4ccccc4c3)c3ccc4oc5cc6ccccc6c(-c6ccc7c8c9ccccc9ccc8n(-c8ccccc8)c7c6)c5c4c3)c21 ; c1ccc(N(c2ccccc2)c2ccc(-c3ccc4oc5cc6ccccc6c(-c6cc7c(c8ccccc68)c6ccccc6n7-c6ccccc6)c5c4c3)cc2)cc1]

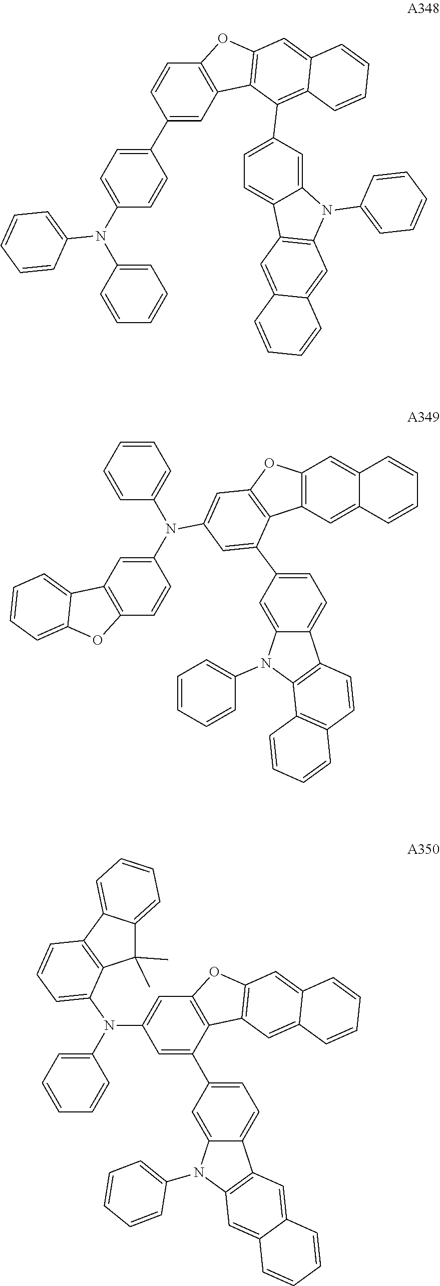 [SMILES: c1ccc(N(c2ccccc2)c2ccc(-c3ccc4oc5cc6ccccc6c(-c6ccc7c8cc9ccccc9cc8n(-c8ccccc8)c7c6)c5c4c3)cc2)cc1 ; c1ccc(N(c2cc(-c3ccc4c5ccc6ccccc6c5n(-c5ccccc5)c4c3)c3c(c2)oc2cc4ccccc4cc23)c2ccc3oc4ccccc4c3c2)cc1 ; CC1(C)c2ccccc2-c2cccc(N(c3ccccc3)c3cc(-c4ccc5c6cc7ccccc7cc6n(-c6ccccc6)c5c4)c4c(c3)oc3cc5ccccc5cc34)c21]

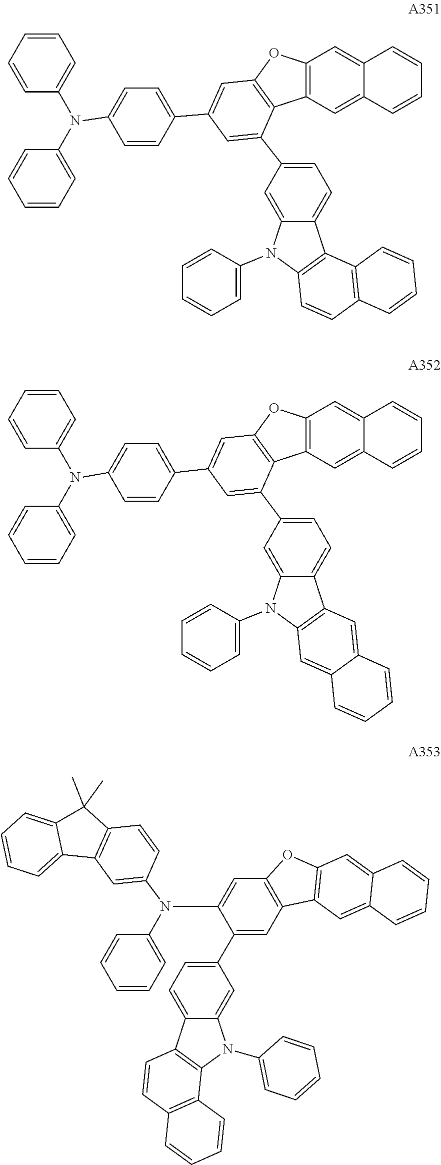 [SMILES: c1ccc(N(c2ccccc2)c2ccc(-c3cc(-c4ccc5c6c7ccccc7ccc6n(-c6ccccc6)c5c4)c4c(c3)oc3cc5ccccc5cc34)cc2)cc1 ; c1ccc(N(c2ccccc2)c2ccc(-c3cc(-c4ccc5c6cc7ccccc7cc6n(-c6ccccc6)c5c4)c4c(c3)oc3cc5ccccc5cc34)cc2)cc1 ; CC1(C)c2ccccc2-c2cc(N(c3ccccc3)c3cc4oc5cc6ccccc6cc5c4cc3-c3ccc4c5ccc6ccccc6c5n(-c5ccccc5)c4c3)ccc21]

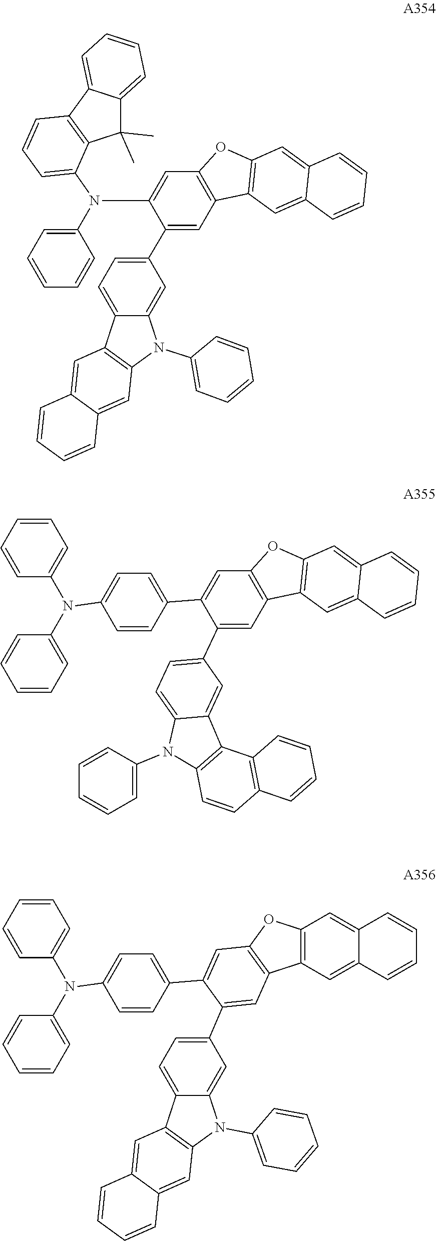 [SMILES: CC1(C)c2ccccc2-c2cccc(N(c3ccccc3)c3cc4oc5cc6ccccc6cc5c4cc3-c3ccc4c5cc6ccccc6cc5n(-c5ccccc5)c4c3)c21 ; c1ccc(N(c2ccccc2)c2ccc(-c3cc4oc5cc6ccccc6cc5c4cc3-c3ccc4c(c3)c3c5ccccc5ccc3n4-c3ccccc3)cc2)cc1 ; c1ccc(N(c2ccccc2)c2ccc(-c3cc4oc5cc6ccccc6cc5c4cc3-c3ccc4c5cc6ccccc6cc5n(-c5ccccc5)c4c3)cc2)cc1]

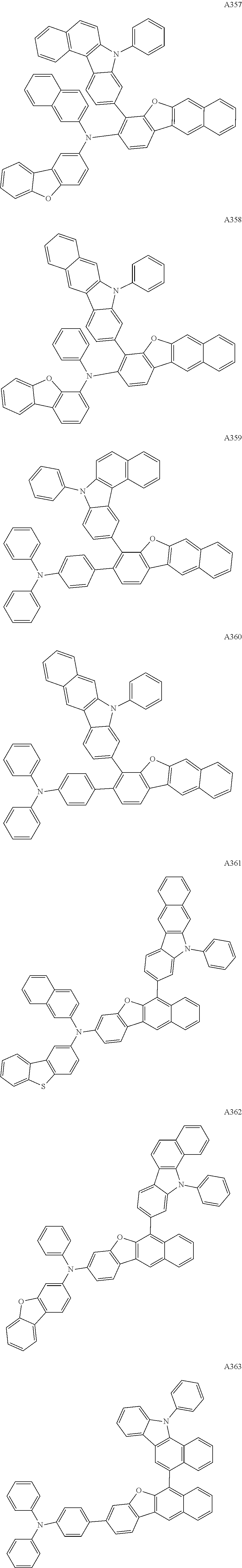

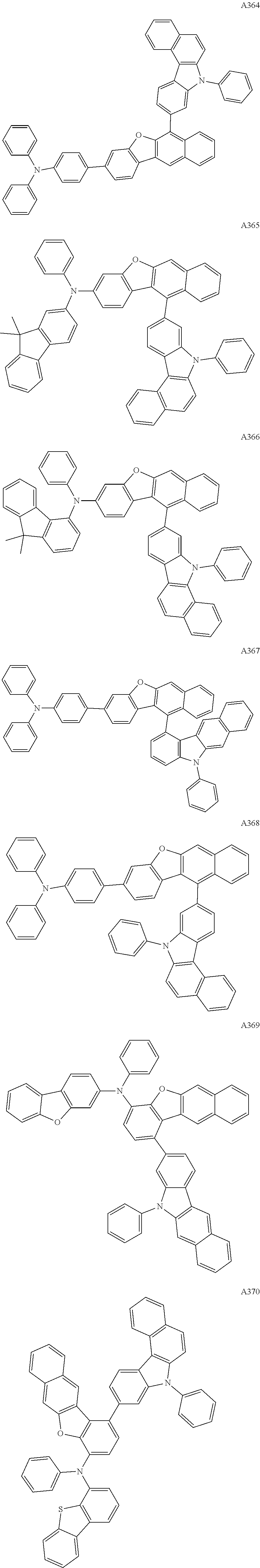

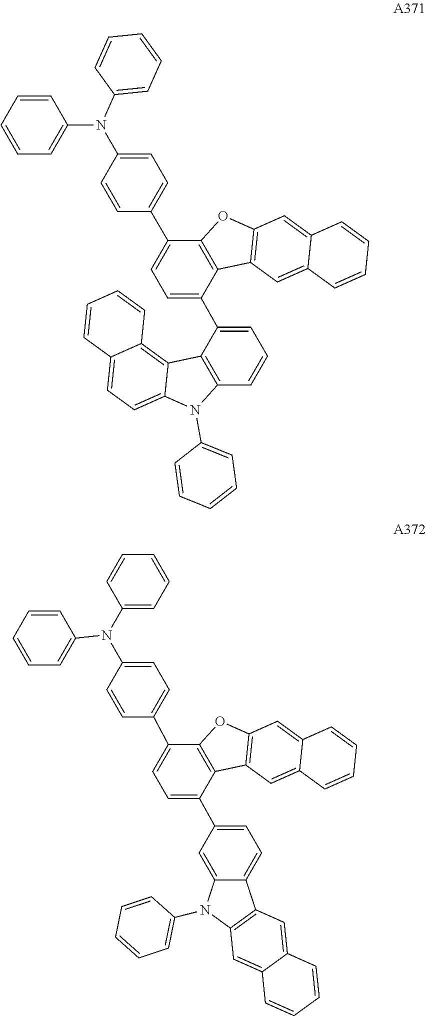 [SMILES: c1ccc(N(c2ccccc2)c2ccc(-c3ccc(-c4cccc5c4c4c6ccccc6ccc4n5-c4ccccc4)c4c3oc3cc5ccccc5cc34)cc2)cc1 ; c1ccc(N(c2ccccc2)c2ccc(-c3ccc(-c4ccc5c6cc7ccccc7cc6n(-c6ccccc6)c5c4)c4c3oc3cc5ccccc5cc34)cc2)cc1]

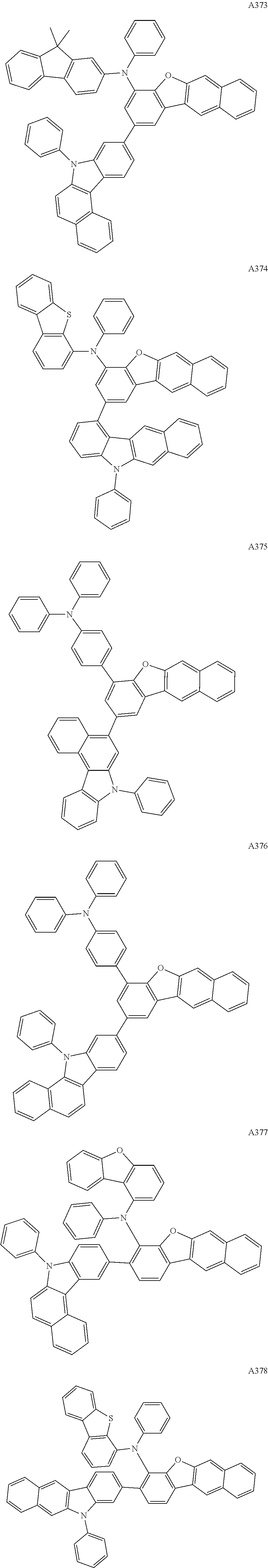

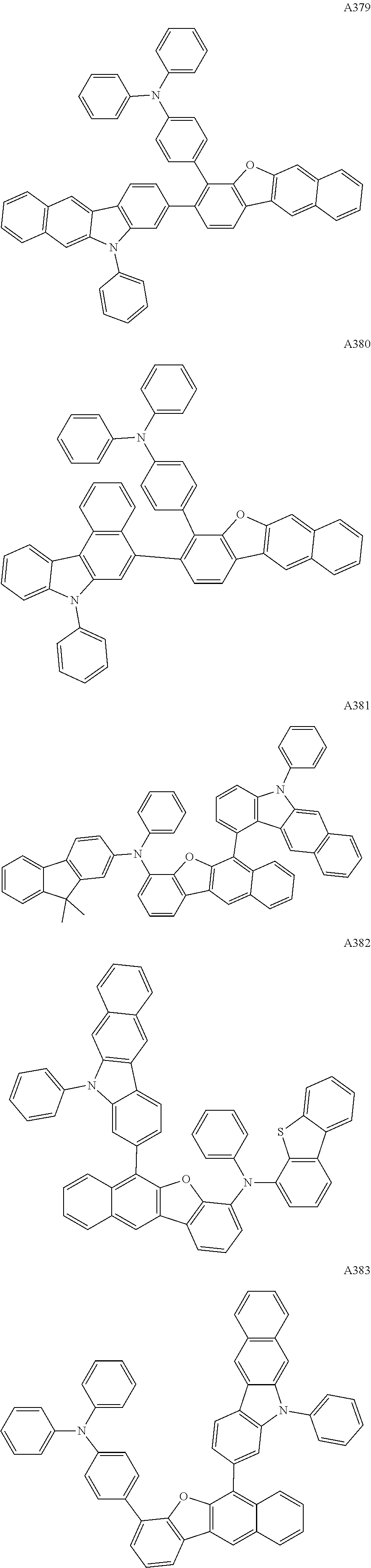 [SMILES: c1ccc(N(c2ccccc2)c2ccc(-c3c(-c4ccc5c6cc7ccccc7cc6n(-c6ccccc6)c5c4)ccc4c3oc3cc5ccccc5cc34)cc2)cc1 ; c1ccc(N(c2ccccc2)c2ccc(-c3c(-c4cc5c(c6ccccc46)c4ccccc4n5-c4ccccc4)ccc4c3oc3cc5ccccc5cc34)cc2)cc1 ; CC1(C)c2ccccc2-c2ccc(N(c3ccccc3)c3cccc4c3oc3c(-c5cccc6c5c5cc7ccccc7cc5n6-c5ccccc5)c5ccccc5cc34)cc21 ; c1ccc(N(c2cccc3c2oc2c(-c4ccc5c6cc7ccccc7cc6n(-c6ccccc6)c5c4)c4ccccc4cc23)c2cccc3c2sc2ccccc23)cc1 ; c1ccc(N(c2ccccc2)c2ccc(-c3cccc4c3oc3c(-c5ccc6c7cc8ccccc8cc7n(-c7ccccc7)c6c5)c5ccccc5cc34)cc2)cc1]

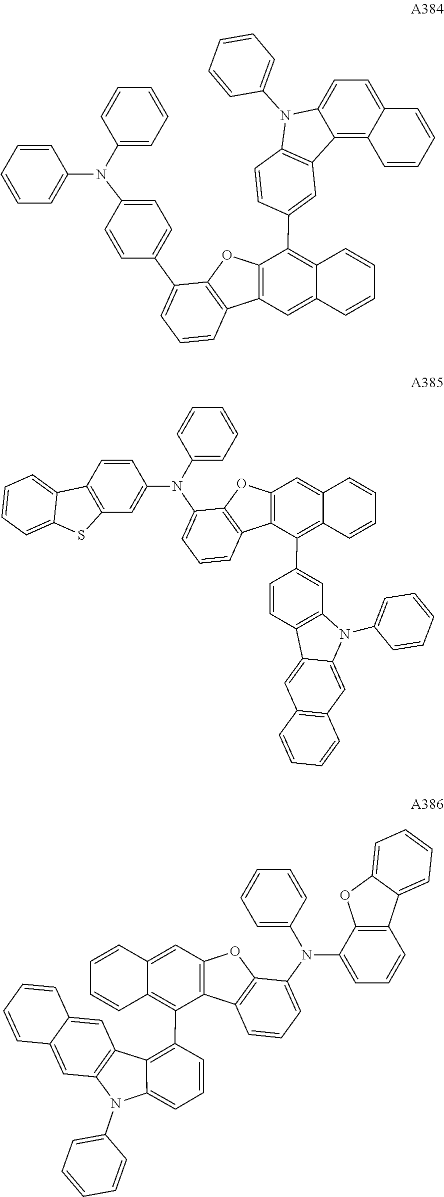 [SMILES: c1ccc(N(c2ccccc2)c2ccc(-c3cccc4c3oc3c(-c5ccc6c(c5)c5c7ccccc7ccc5n6-c5ccccc5)c5ccccc5cc34)cc2)cc1 ; c1ccc(N(c2ccc3c(c2)sc2ccccc23)c2cccc3c2oc2cc4ccccc4c(-c4ccc5c6cc7ccccc7cc6n(-c6ccccc6)c5c4)c23)cc1 ; c1ccc(N(c2cccc3c2oc2ccccc23)c2cccc3c2oc2cc4ccccc4c(-c4cccc5c4c4cc6ccccc6cc4n5-c4ccccc4)c23)cc1]

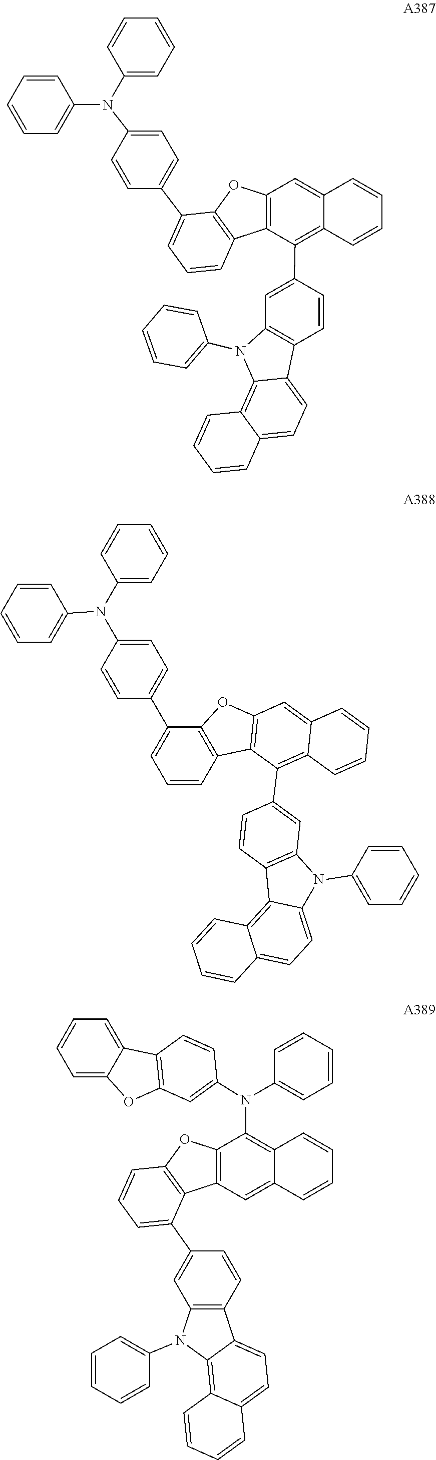 [SMILES: c1ccc(N(c2ccccc2)c2ccc(-c3cccc4c3oc3cc5ccccc5c(-c5ccc6c7ccc8ccccc8c7n(-c7ccccc7)c6c5)c34)cc2)cc1 ; c1ccc(N(c2ccccc2)c2ccc(-c3cccc4c3oc3cc5ccccc5c(-c5ccc6c7c8ccccc8ccc7n(-c7ccccc7)c6c5)c34)cc2)cc1 ; c1ccc(N(c2ccc3c(c2)oc2ccccc23)c2c3ccccc3cc3c2oc2cccc(-c4ccc5c6ccc7ccccc7c6n(-c6ccccc6)c5c4)c23)cc1]

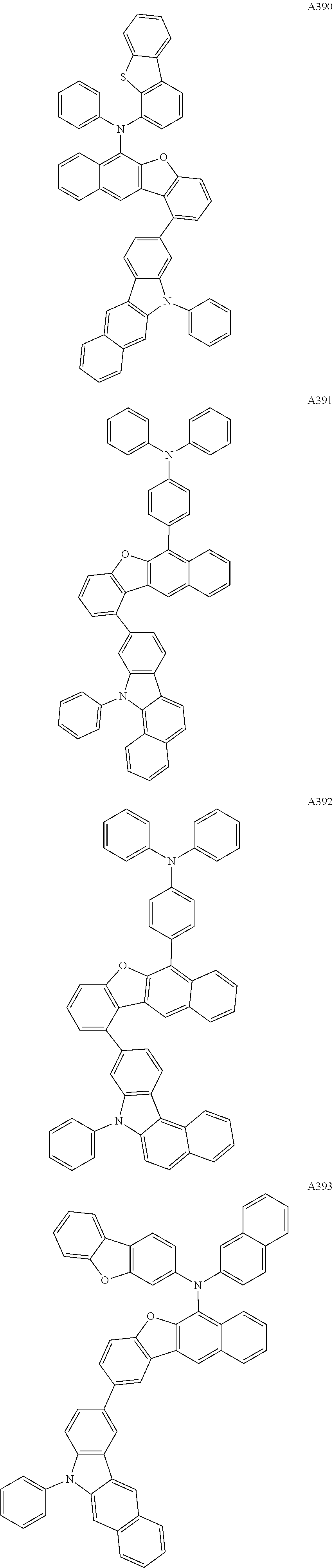

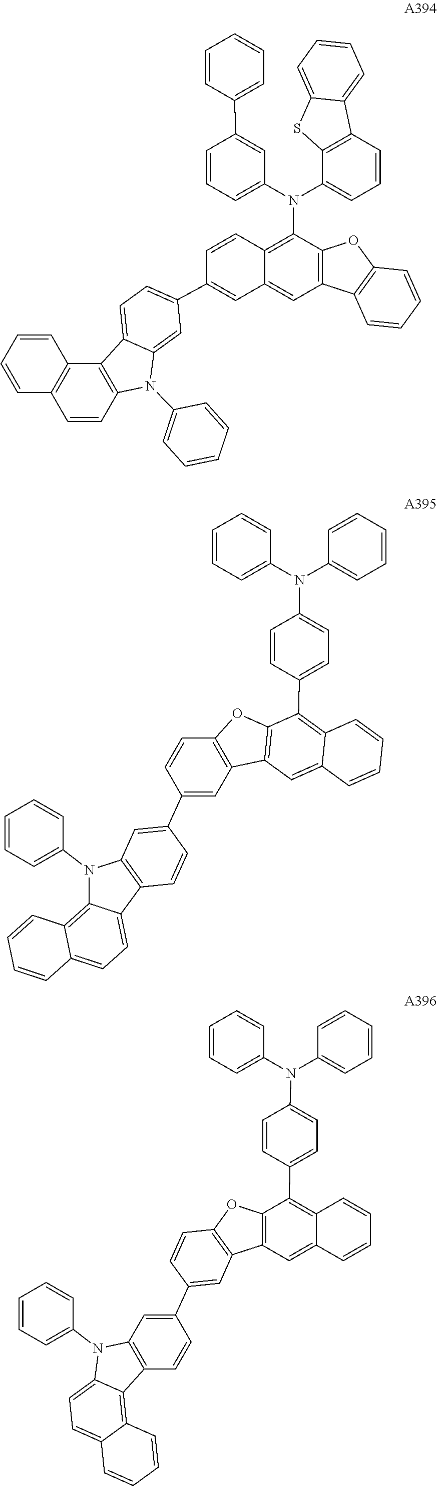 [SMILES: c1ccc(-c2cccc(N(c3c4ccc(-c5ccc6c7c8ccccc8ccc7n(-c7ccccc7)c6c5)cc4cc4c3oc3ccccc34)c3cccc4c3sc3ccccc34)c2)cc1 ; c1ccc(N(c2ccccc2)c2ccc(-c3c4ccccc4cc4c3oc3ccc(-c5ccc6c7ccc8ccccc8c7n(-c7ccccc7)c6c5)cc34)cc2)cc1 ; c1ccc(N(c2ccccc2)c2ccc(-c3c4ccccc4cc4c3oc3ccc(-c5ccc6c7c8ccccc8ccc7n(-c7ccccc7)c6c5)cc34)cc2)cc1]

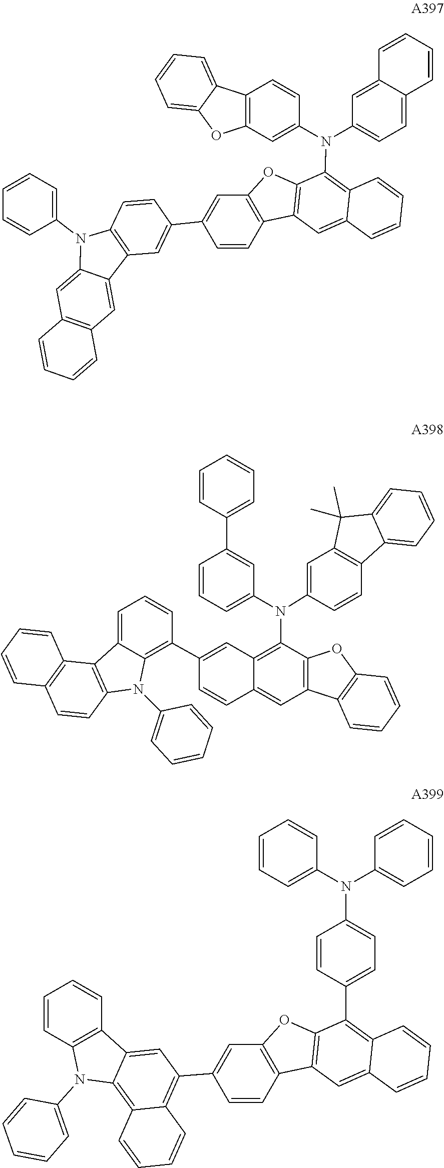 [SMILES: c1ccc(-n2c3ccc(-c4ccc5c(c4)oc4c(N(c6ccc7ccccc7c6)c6ccc7c(c6)oc6ccccc67)c6ccccc6cc45)cc3c3cc4ccccc4cc32)cc1 ; CC1(C)c2ccccc2-c2ccc(N(c3cccc(-c4ccccc4)c3)c3c4cc(-c5cccc6c7c8ccccc8ccc7n(-c7ccccc7)c56)ccc4cc4c3oc3ccccc34)cc21 ; c1ccc(N(c2ccccc2)c2ccc(-c3c4ccccc4cc4c3oc3cc(-c5cc6c7ccccc7n(-c7ccccc7)c6c6ccccc56)ccc34)cc2)cc1]

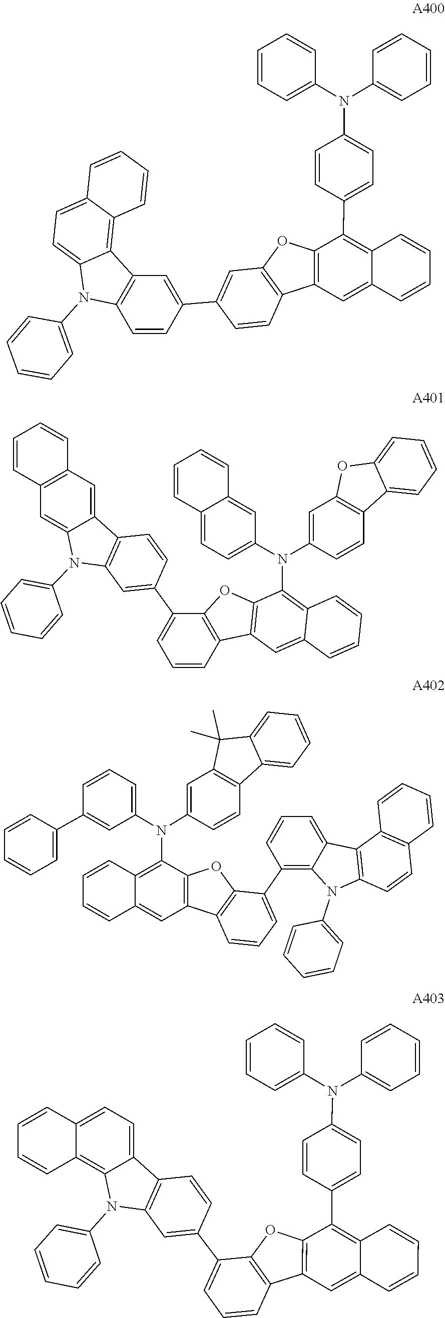

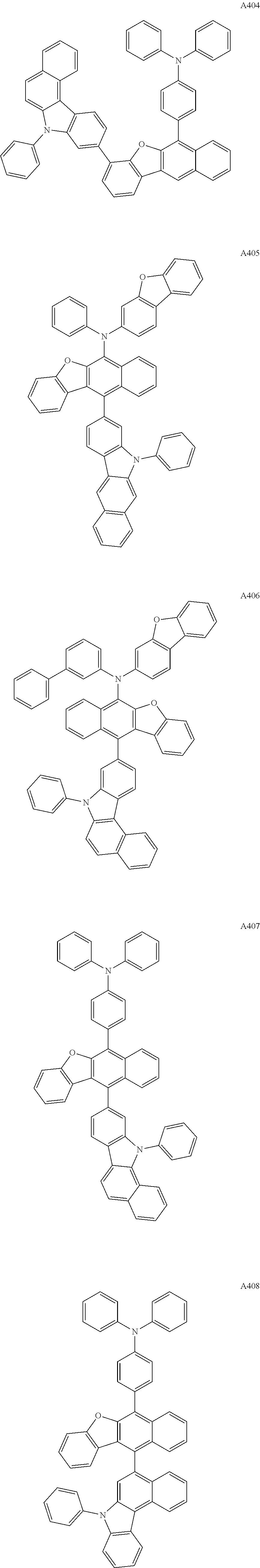

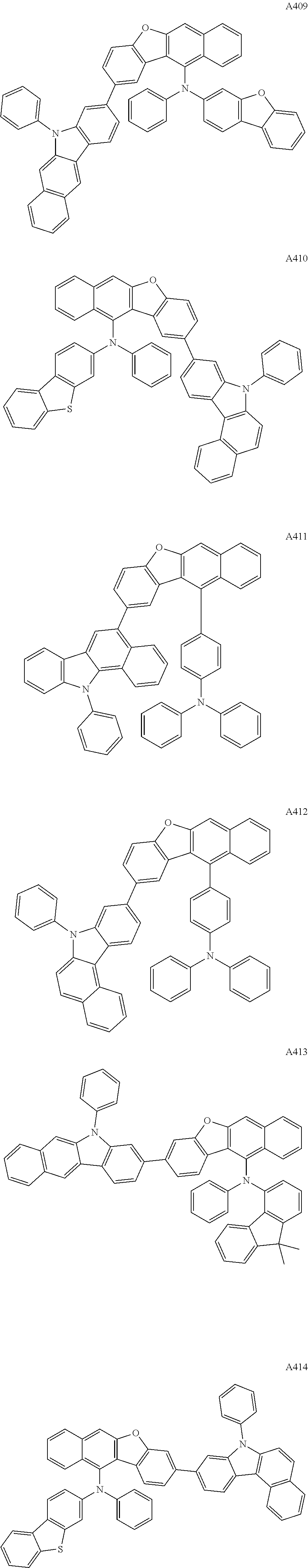

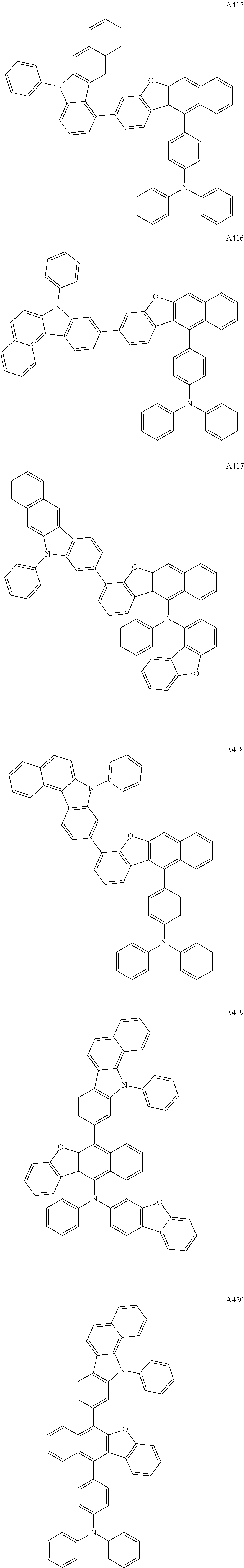

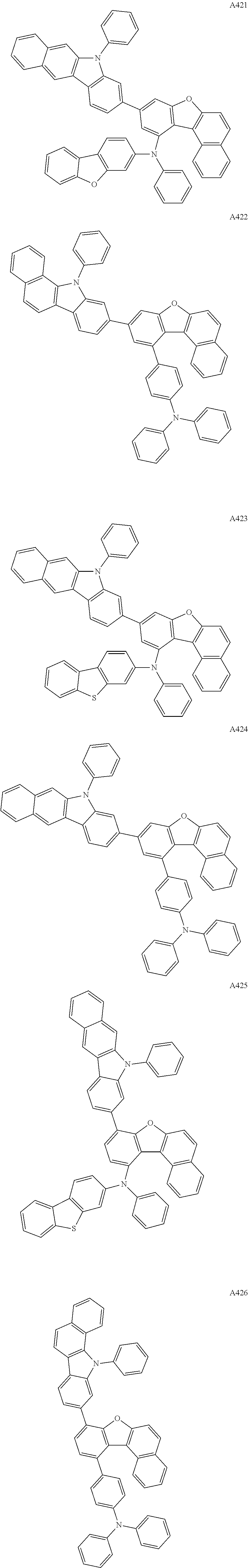

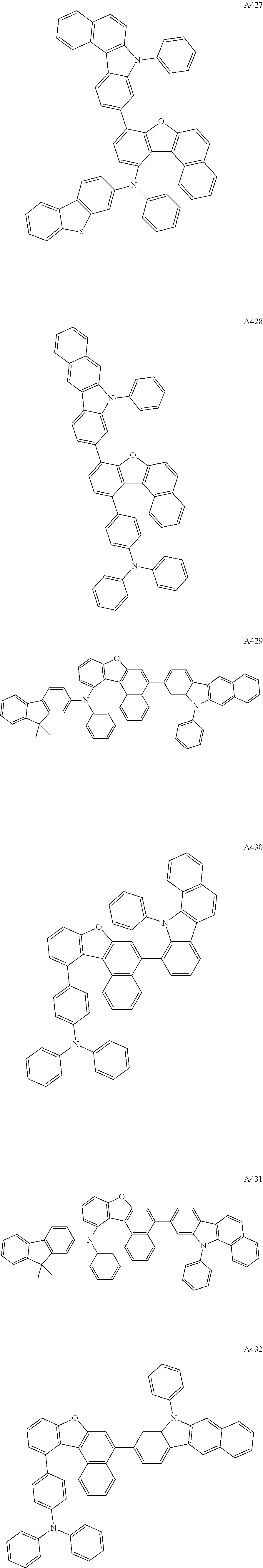

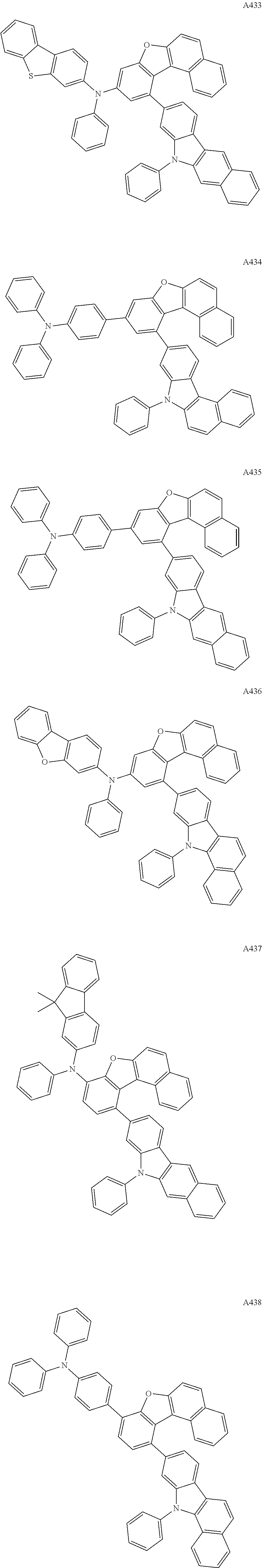

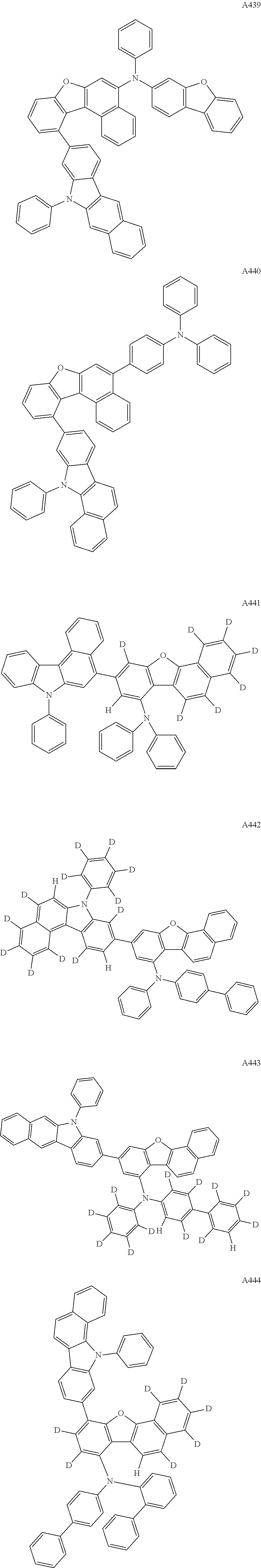

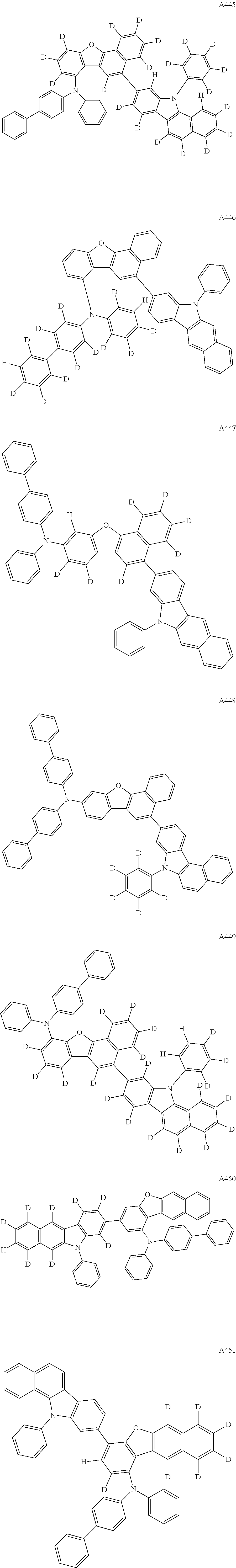

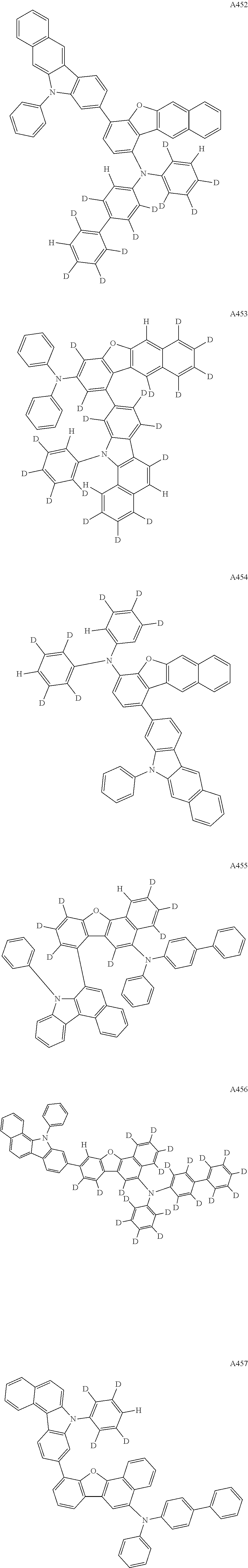

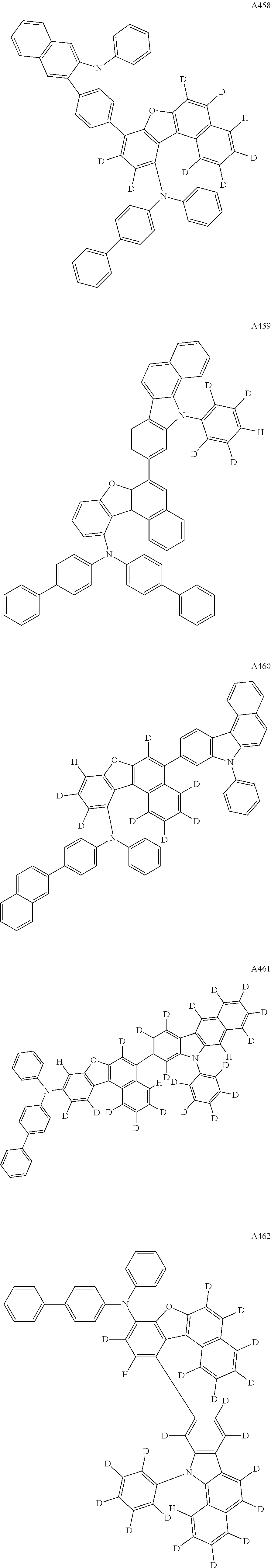

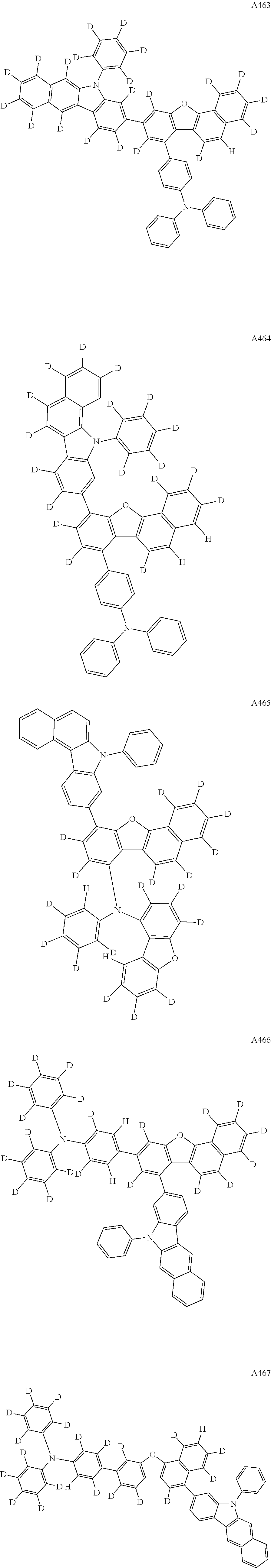

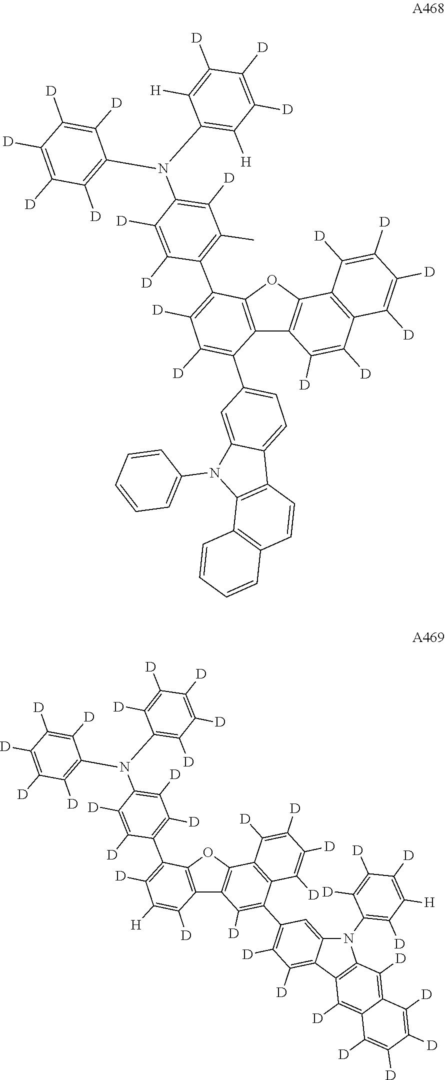 [SMILES: [2H]c1cc(N(c2c([2H])c([2H])c([2H])c([2H])c2[2H])c2c([2H])c([2H])c(-c3c([2H])c([2H])c(-c4ccc5c6ccc7ccccc7c6n(-c6ccccc6)c5c4)c4c3oc3c5c([2H])c([2H])c([2H])c([2H])c5c([2H])c([2H])c34)c(C)c2[2H])cc([2H])c1[2H] ; [2H]c1cc([2H])c(-n2c3cc(-c4c([2H])c5c(oc6c(-c7c([2H])c([2H])c(N(c8c([2H])c([2H])c([2H])c([2H])c8[2H])c8c([2H])c([2H])c([2H])c([2H])c8[2H])c([2H])c7[2H])c([2H])cc([2H])c65)c5c([2H])c([2H])c([2H])c([2H])c45)c([2H])c([2H])c3c3c([2H])c4c([2H])c([2H])c([2H])c([2H])c4c([2H])c32)c([2H])c1[2H]]

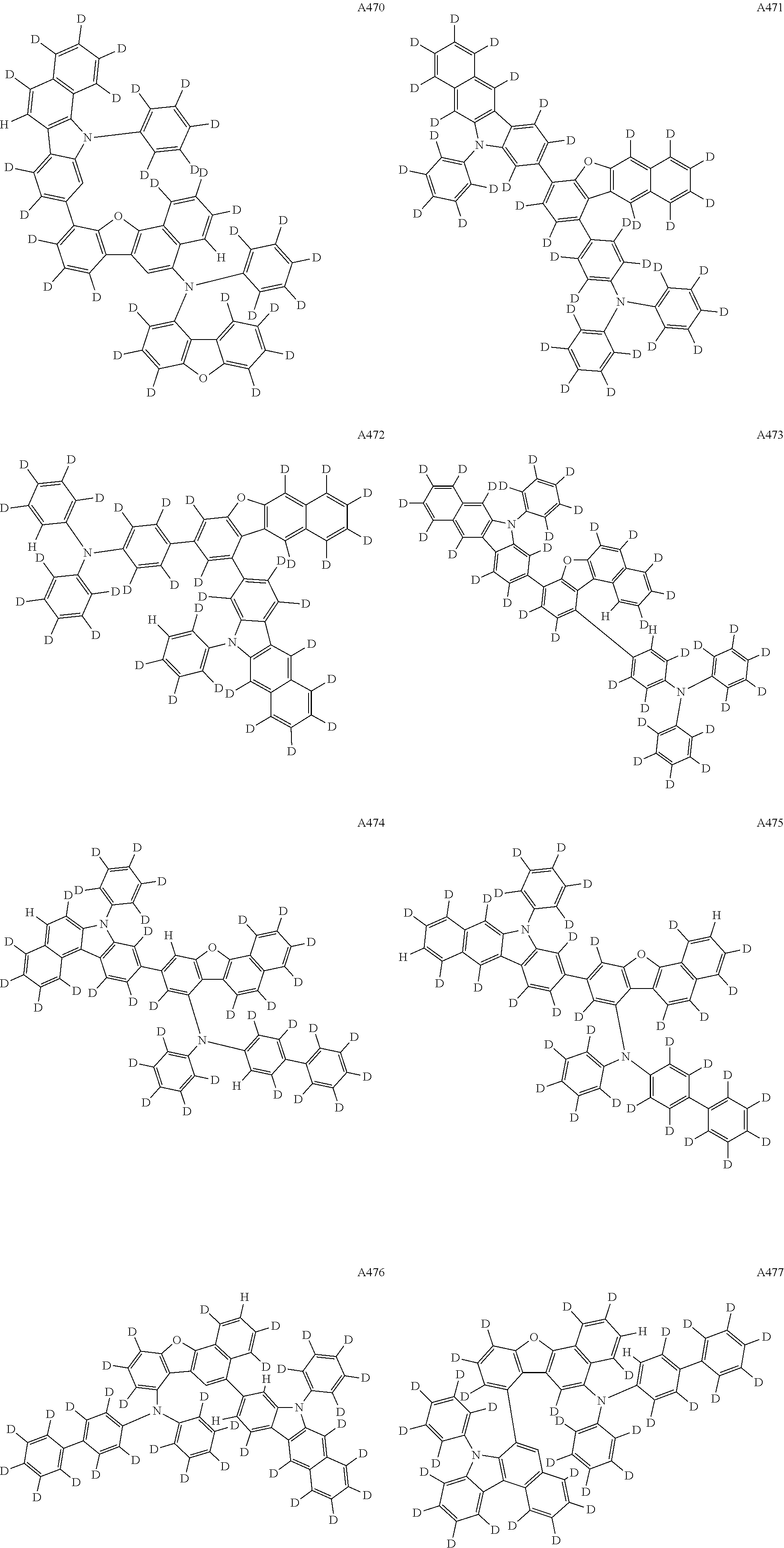

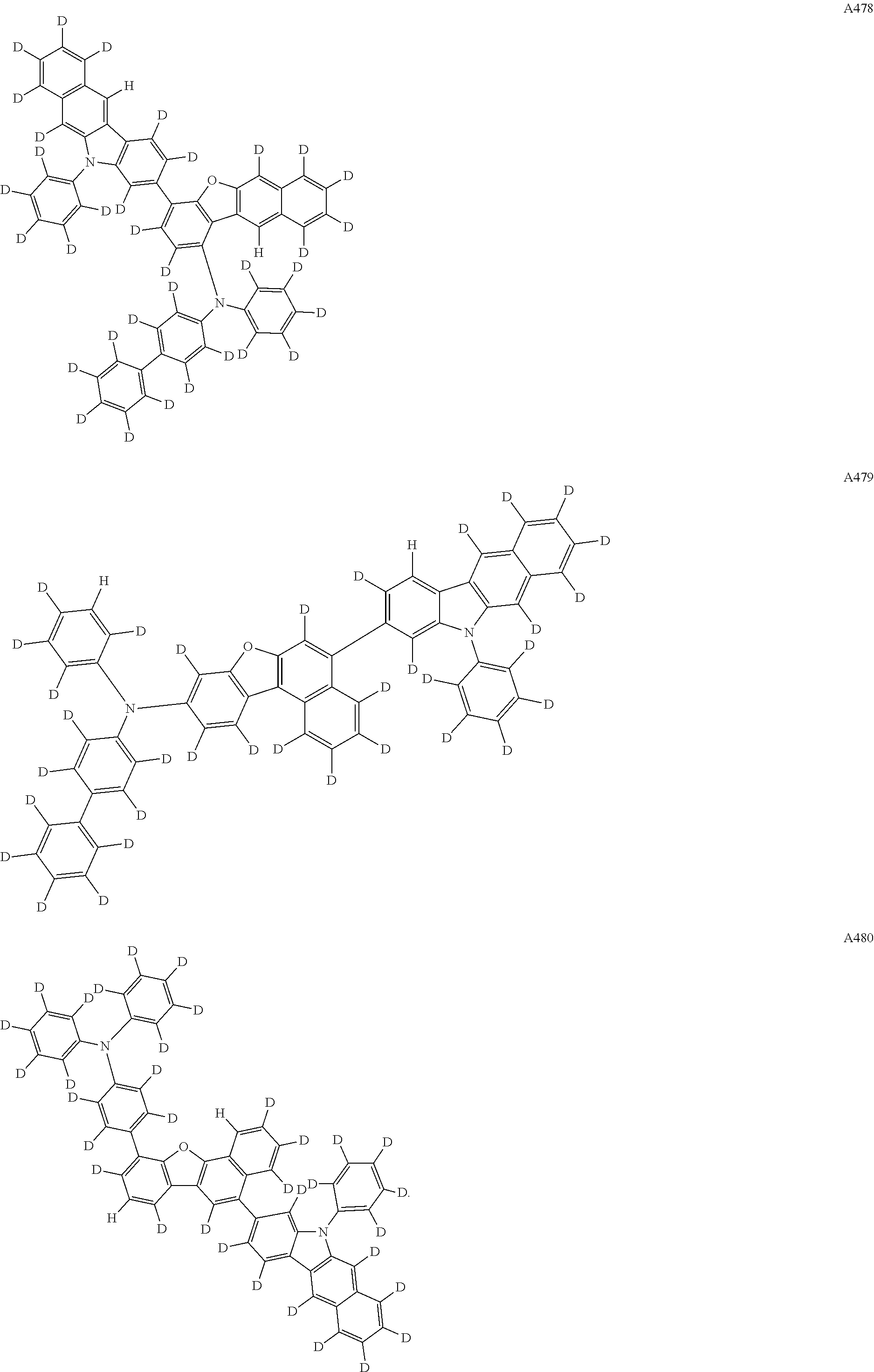

9. A light-emitting device comprising:
a first electrode;
a second electrode; and
one or more organic layers interposed between the first electrode and the second electrode,
wherein one or more of the organic layers comprises the heterocyclic compound according to claim 1.

10. The light-emitting device according to claim 9, wherein the organic layer comprises an emission layer, and
the emission layer comprises a dopant material and a host material, and the host material comprises the heterocyclic compound.

11. The light-emitting device according to claim 10, wherein the organic layer further comprises a hole transport layer disposed between the first electrode and the emission layer, and an electron transport layer disposed between the emission layer and the second electrode.

12. The light-emitting device according to claim 10, wherein the host material further comprises a compound represented by Formula 4: 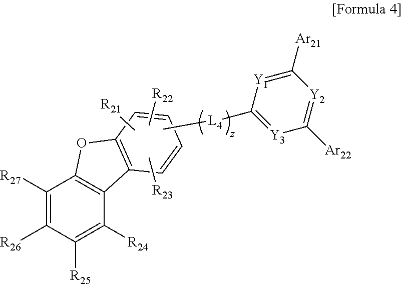
in Formula 4, any one of R₂₁ to R₂₇ is a substituted or unsubstituted aryl group having 6 to 60 carbon atoms, or a substituted or unsubstituted heteroaryl group having 2 to 60 carbon atoms,
the remaining groups of R₂₁ to R₂₇ are each independently selected from the group consisting of hydrogen, deuterium, a halogen, a cyano group, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted alkoxy group having 1 to 60 carbon atoms, a substituted or unsubstituted cycloalkyl group having 3 to 60 carbon atoms, a substituted or unsubstituted heterocycloalkyl group having 2 to 60 carbon atoms, a substituted or unsubstituted aryl group having 6 to 60 carbon atoms, a substituted or unsubstituted heteroaryl group having 2 to 60 carbon atoms, a phosphine oxide group, and a silyl group, or two or more adjacent groups are linked to form a substituted or unsubstituted aromatic hydrocarbon ring having 6 to 60 carbon atoms or a substituted or unsubstituted heterocycle having 2 to 60 carbon atoms,
L₄ is a direct linkage, a substituted or unsubstituted arylene group having 6 to 60 carbon atoms, or a substituted or unsubstituted heteroarylene group having 2 to 60 carbon atoms;
z is an integer of 0 to 5;
when z is an integer of 2 to 5, a plurality of L₄ groups are the same or different;
Y₁ to Y₃ are each independently CH, nitrogen (N), oxygen (O), or sulfur(S); and at least two of Y₁ to Y₃ are nitrogen; and
Ar₂₁ and Ar₂₂ are each independently a substituted or unsubstituted aryl group having 6 to 60 carbon atoms, or a substituted or unsubstituted heteroaryl group having 2 to 60 carbon atoms.

13. The light-emitting device according to claim 12, wherein the compound represented by Formula 4 comprises one or more compounds represented by any one of Formulae below:

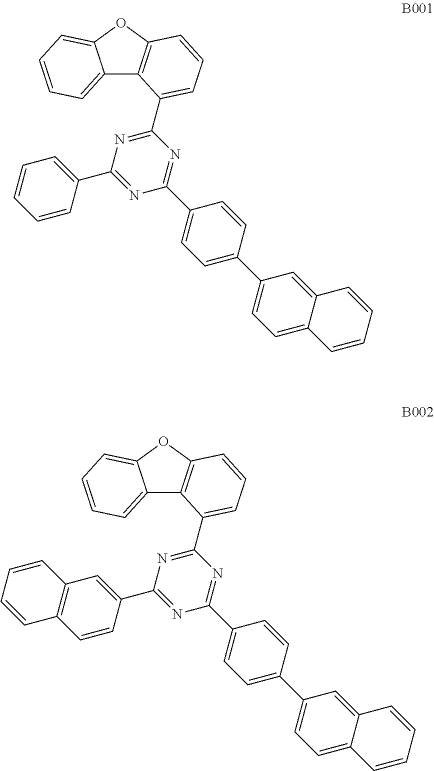 [SMILES: c1ccc(-c2nc(-c3ccc(-c4ccc5ccccc5c4)cc3)nc(-c3cccc4oc5ccccc5c34)n2)cc1 ; c1ccc2cc(-c3ccc(-c4nc(-c5ccc6ccccc6c5)nc(-c5cccc6oc7ccccc7c56)n4)cc3)ccc2c1]

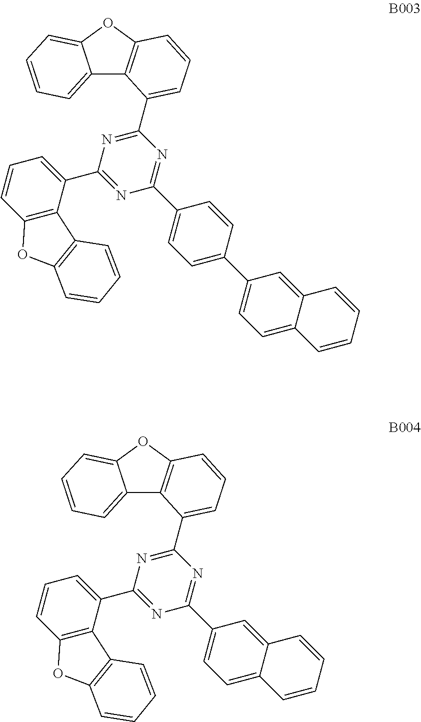 [SMILES: c1ccc2cc(-c3ccc(-c4nc(-c5cccc6oc7ccccc7c56)nc(-c5cccc6oc7ccccc7c56)n4)cc3)ccc2c1 ; c1ccc2cc(-c3nc(-c4cccc5oc6ccccc6c45)nc(-c4cccc5oc6ccccc6c45)n3)ccc2c1]

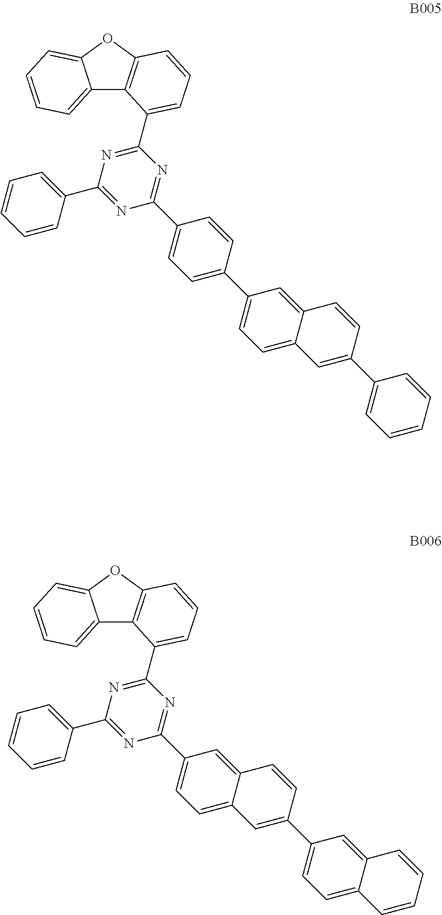 [SMILES: c1ccc(-c2ccc3cc(-c4ccc(-c5nc(-c6ccccc6)nc(-c6cccc7oc8ccccc8c67)n5)cc4)ccc3c2)cc1 ; c1ccc(-c2nc(-c3ccc4cc(-c5ccc6ccccc6c5)ccc4c3)nc(-c3cccc4oc5ccccc5c34)n2)cc1]

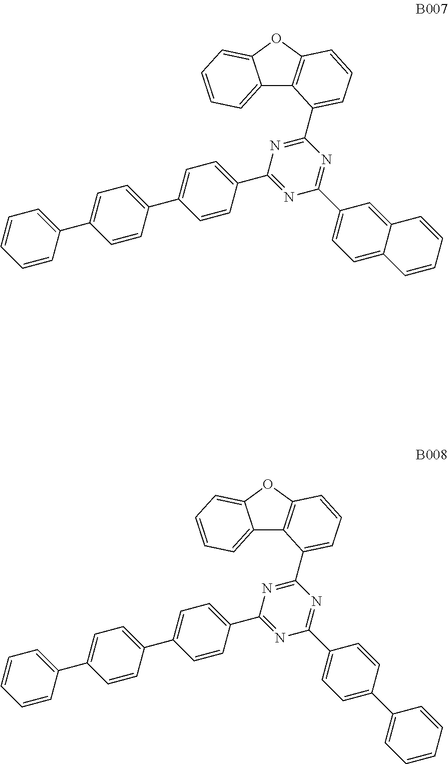 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3ccc(-c4nc(-c5ccc(-c6ccccc6)cc5)nc(-c5cccc6oc7ccccc7c56)n4)cc3)cc2)cc1]

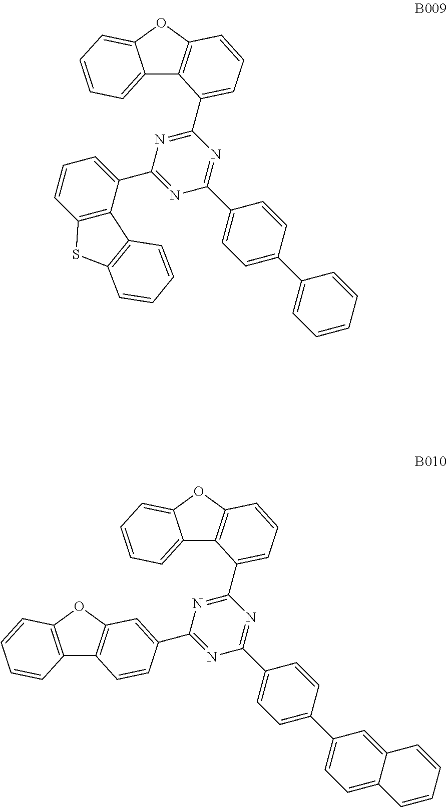 [SMILES: c1ccc(-c2ccc(-c3nc(-c4cccc5oc6ccccc6c45)nc(-c4cccc5sc6ccccc6c45)n3)cc2)cc1 ; c1ccc2cc(-c3ccc(-c4nc(-c5ccc6c(c5)oc5ccccc56)nc(-c5cccc6oc7ccccc7c56)n4)cc3)ccc2c1]

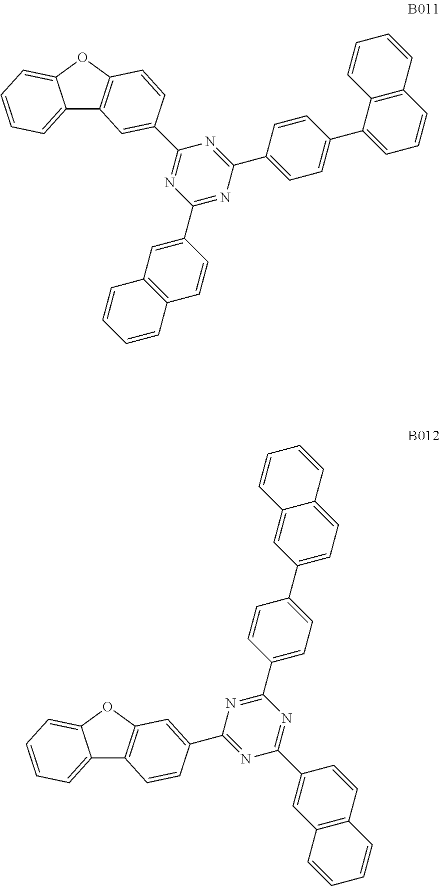 [SMILES: c1ccc2cc(-c3nc(-c4ccc(-c5cccc6ccccc56)cc4)nc(-c4ccc5oc6ccccc6c5c4)n3)ccc2c1 ; c1ccc2cc(-c3ccc(-c4nc(-c5ccc6ccccc6c5)nc(-c5ccc6c(c5)oc5ccccc56)n4)cc3)ccc2c1]

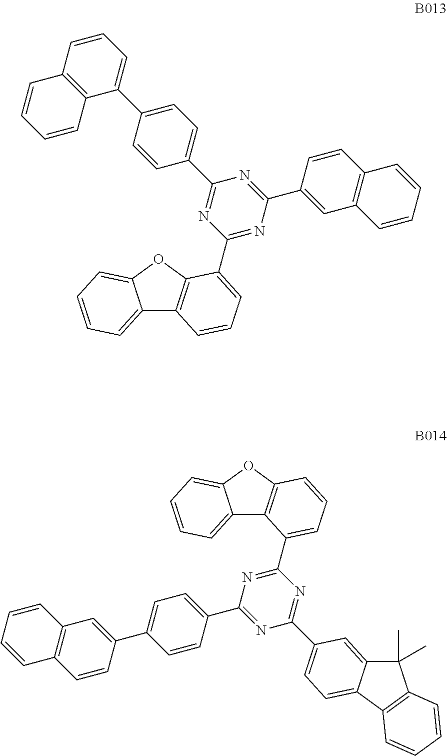 [SMILES: c1ccc2cc(-c3nc(-c4ccc(-c5cccc6ccccc56)cc4)nc(-c4cccc5c4oc4ccccc45)n3)ccc2c1 ; CC1(C)c2ccccc2-c2ccc(-c3nc(-c4ccc(-c5ccc6ccccc6c5)cc4)nc(-c4cccc5oc6ccccc6c45)n3)cc21]

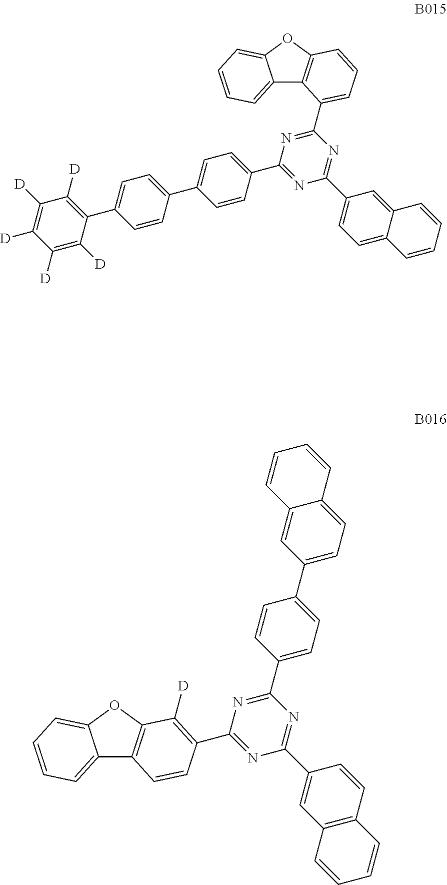 [SMILES: [2H]c1c([2H])c([2H])c(-c2ccc(-c3ccc(-c4nc(-c5ccc6ccccc6c5)nc(-c5cccc6oc7ccccc7c56)n4)cc3)cc2)c([2H])c1[2H] ; [2H]c1c(-c2nc(-c3ccc(-c4ccc5ccccc5c4)cc3)nc(-c3ccc4ccccc4c3)n2)ccc2c1oc1ccccc12]

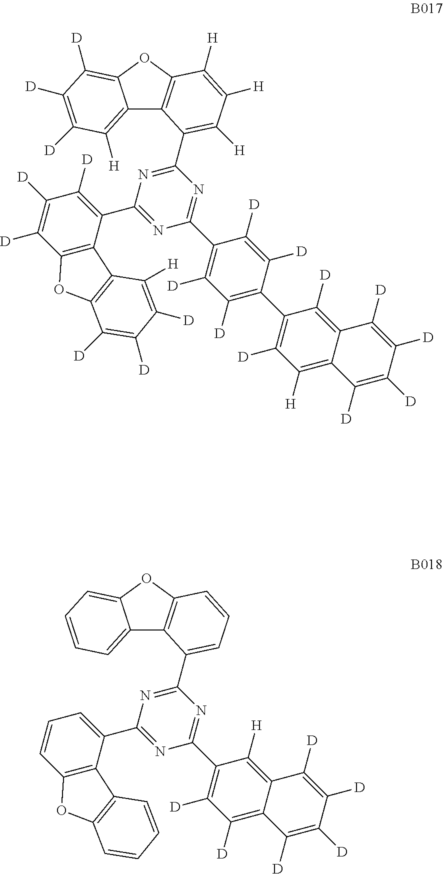 [SMILES: [2H]c1cc2c(oc3cccc(-c4nc(-c5c([2H])c([2H])c(-c6c([2H])cc7c([2H])c([2H])c([2H])c([2H])c7c6[2H])c([2H])c5[2H])nc(-c5c([2H])c([2H])c([2H])c6oc7c([2H])c([2H])c([2H])cc7c56)n4)c32)c([2H])c1[2H] ; [2H]c1c([2H])c([2H])c2c([2H])c([2H])c(-c3nc(-c4cccc5oc6ccccc6c45)nc(-c4cccc5oc6ccccc6c45)n3)cc2c1[2H]]

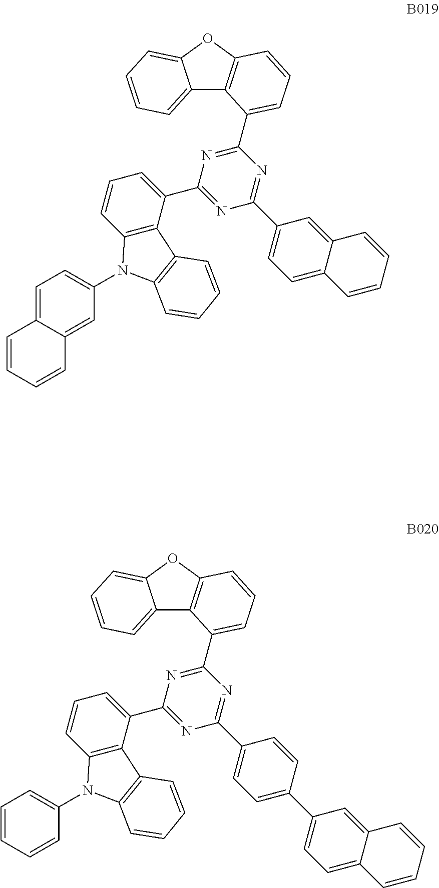 [SMILES: c1ccc2cc(-c3nc(-c4cccc5oc6ccccc6c45)nc(-c4cccc5c4c4ccccc4n5-c4ccc5ccccc5c4)n3)ccc2c1 ; c1ccc(-n2c3ccccc3c3c(-c4nc(-c5ccc(-c6ccc7ccccc7c6)cc5)nc(-c5cccc6oc7ccccc7c56)n4)cccc32)cc1]

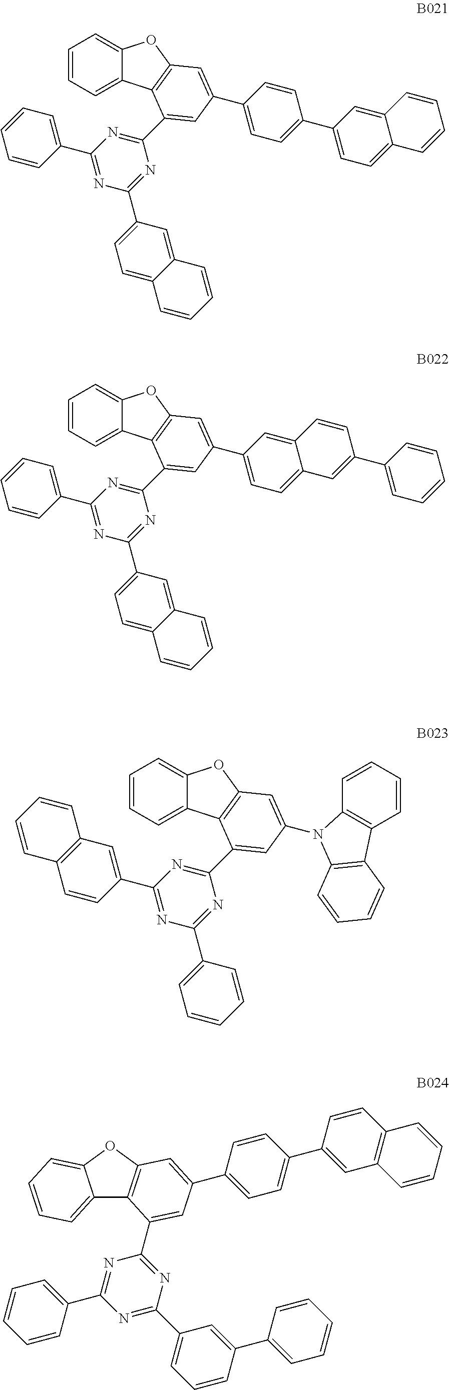 [SMILES: c1ccc(-c2nc(-c3ccc4ccccc4c3)nc(-c3cc(-c4ccc(-c5ccc6ccccc6c5)cc4)cc4oc5ccccc5c34)n2)cc1 ; c1ccc(-c2ccc3cc(-c4cc(-c5nc(-c6ccccc6)nc(-c6ccc7ccccc7c6)n5)c5c(c4)oc4ccccc45)ccc3c2)cc1 ; c1ccc(-c2nc(-c3ccc4ccccc4c3)nc(-c3cc(-n4c5ccccc5c5ccccc54)cc4oc5ccccc5c34)n2)cc1 ; c1ccc(-c2cccc(-c3nc(-c4ccccc4)nc(-c4cc(-c5ccc(-c6ccc7ccccc7c6)cc5)cc5oc6ccccc6c45)n3)c2)cc1]

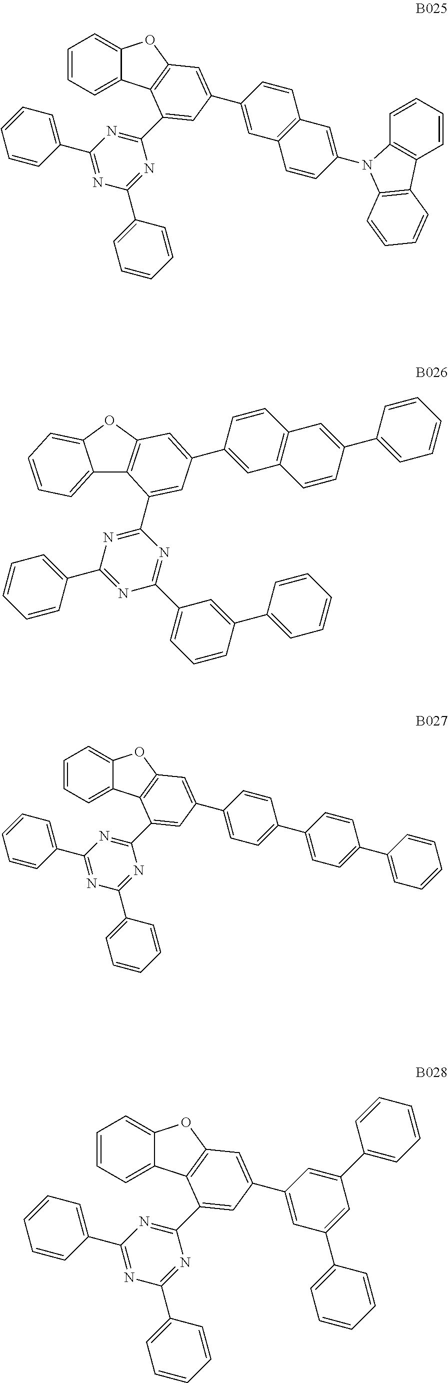 [SMILES: c1ccc(-c2nc(-c3ccccc3)nc(-c3cc(-c4ccc5cc(-n6c7ccccc7c7ccccc76)ccc5c4)cc4oc5ccccc5c34)n2)cc1 ; c1ccc(-c2cccc(-c3nc(-c4ccccc4)nc(-c4cc(-c5ccc6cc(-c7ccccc7)ccc6c5)cc5oc6ccccc6c45)n3)c2)cc1 ; c1ccc(-c2ccc(-c3ccc(-c4cc(-c5nc(-c6ccccc6)nc(-c6ccccc6)n5)c5c(c4)oc4ccccc45)cc3)cc2)cc1 ; c1ccc(-c2cc(-c3ccccc3)cc(-c3cc(-c4nc(-c5ccccc5)nc(-c5ccccc5)n4)c4c(c3)oc3ccccc34)c2)cc1]

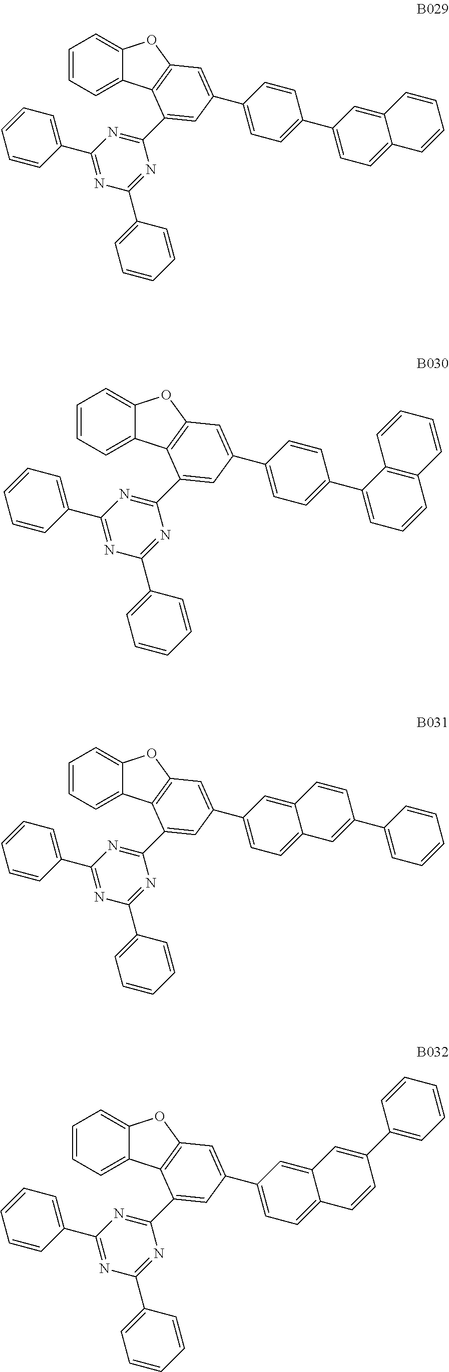 [SMILES: c1ccc(-c2nc(-c3ccccc3)nc(-c3cc(-c4ccc(-c5ccc6ccccc6c5)cc4)cc4oc5ccccc5c34)n2)cc1 ; c1ccc(-c2nc(-c3ccccc3)nc(-c3cc(-c4ccc(-c5cccc6ccccc56)cc4)cc4oc5ccccc5c34)n2)cc1 ; c1ccc(-c2ccc3cc(-c4cc(-c5nc(-c6ccccc6)nc(-c6ccccc6)n5)c5c(c4)oc4ccccc45)ccc3c2)cc1 ; c1ccc(-c2ccc3ccc(-c4cc(-c5nc(-c6ccccc6)nc(-c6ccccc6)n5)c5c(c4)oc4ccccc45)cc3c2)cc1]

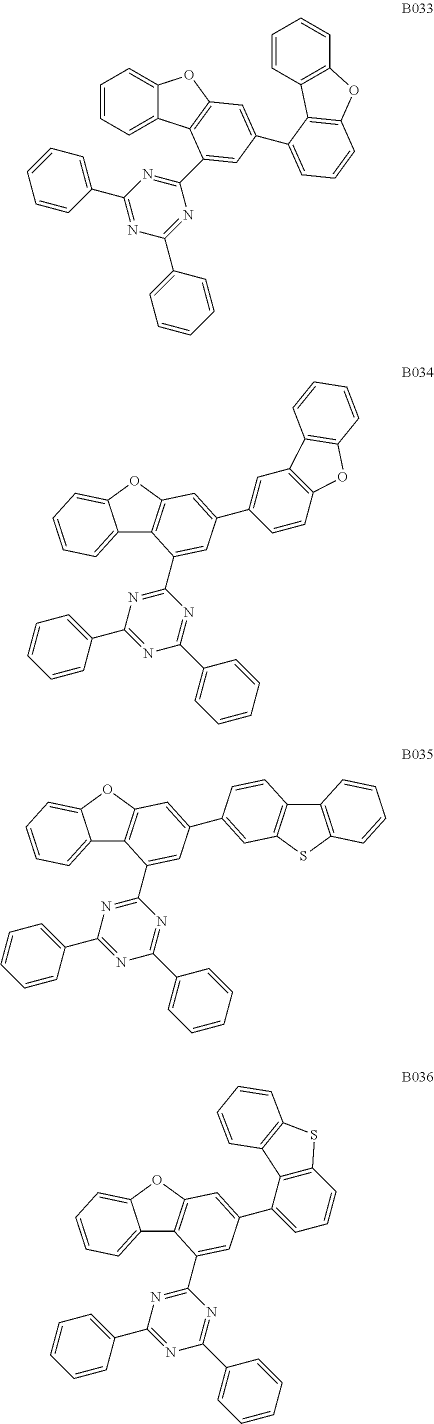 [SMILES: c1ccc(-c2nc(-c3ccccc3)nc(-c3cc(-c4cccc5oc6ccccc6c45)cc4oc5ccccc5c34)n2)cc1 ; c1ccc(-c2nc(-c3ccccc3)nc(-c3cc(-c4ccc5oc6ccccc6c5c4)cc4oc5ccccc5c34)n2)cc1 ; c1ccc(-c2nc(-c3ccccc3)nc(-c3cc(-c4ccc5c(c4)sc4ccccc45)cc4oc5ccccc5c34)n2)cc1 ; c1ccc(-c2nc(-c3ccccc3)nc(-c3cc(-c4cccc5sc6ccccc6c45)cc4oc5ccccc5c34)n2)cc1]

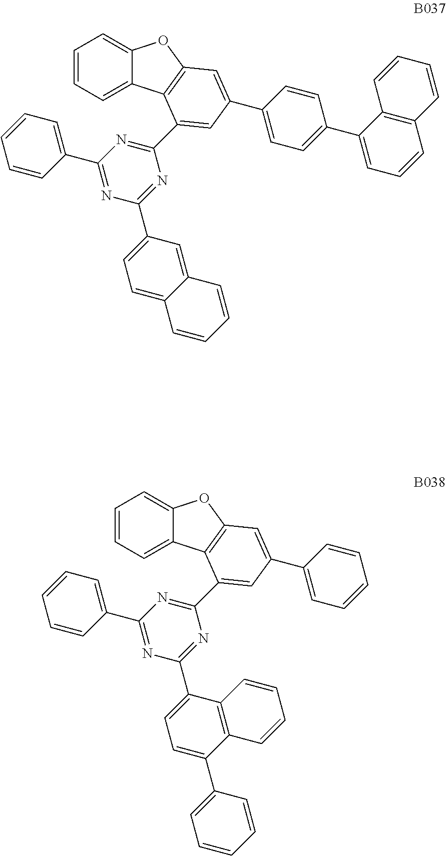 [SMILES: c1ccc(-c2nc(-c3ccc4ccccc4c3)nc(-c3cc(-c4ccc(-c5cccc6ccccc56)cc4)cc4oc5ccccc5c34)n2)cc1 ; c1ccc(-c2cc(-c3nc(-c4ccccc4)nc(-c4ccc(-c5ccccc5)c5ccccc45)n3)c3c(c2)oc2ccccc23)cc1]

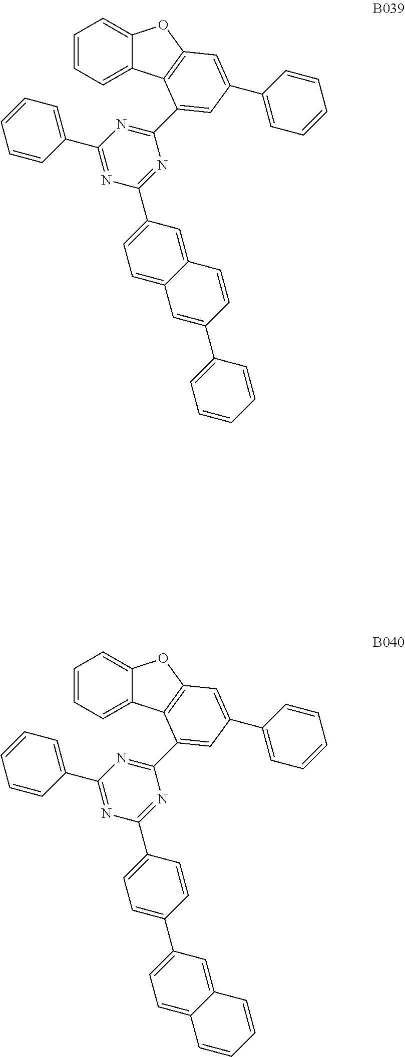 [SMILES: c1ccc(-c2ccc3cc(-c4nc(-c5ccccc5)nc(-c5cc(-c6ccccc6)cc6oc7ccccc7c56)n4)ccc3c2)cc1 ; c1ccc(-c2cc(-c3nc(-c4ccccc4)nc(-c4ccc(-c5ccc6ccccc6c5)cc4)n3)c3c(c2)oc2ccccc23)cc1]

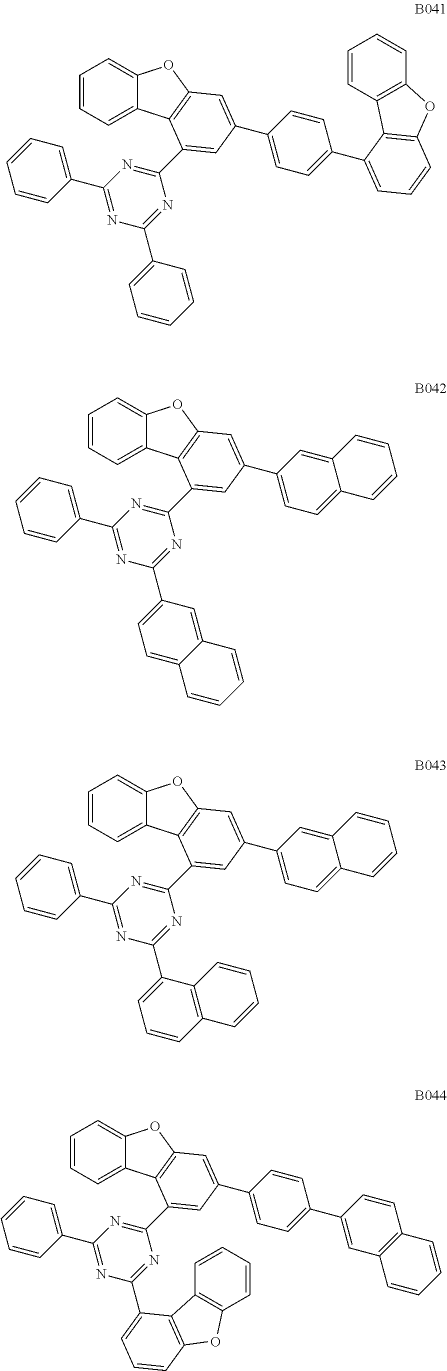 [SMILES: c1ccc(-c2nc(-c3ccccc3)nc(-c3cc(-c4ccc(-c5cccc6oc7ccccc7c56)cc4)cc4oc5ccccc5c34)n2)cc1 ; c1ccc(-c2nc(-c3ccc4ccccc4c3)nc(-c3cc(-c4ccc5ccccc5c4)cc4oc5ccccc5c34)n2)cc1 ; c1ccc(-c2nc(-c3cccc4ccccc34)nc(-c3cc(-c4ccc5ccccc5c4)cc4oc5ccccc5c34)n2)cc1 ; c1ccc(-c2nc(-c3cccc4oc5ccccc5c34)nc(-c3cc(-c4ccc(-c5ccc6ccccc6c5)cc4)cc4oc5ccccc5c34)n2)cc1]

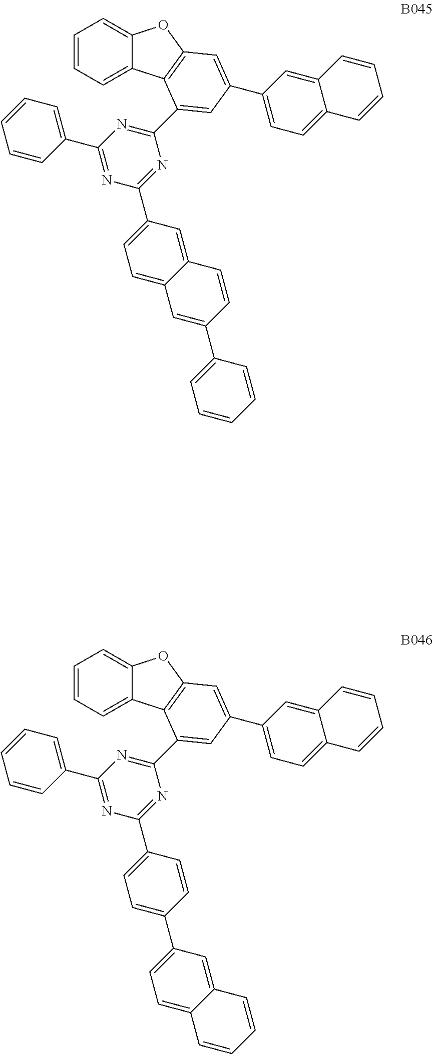 [SMILES: c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2nc(-c3ccc(-c4ccc5ccccc5c4)cc3)nc(-c3cc(-c4ccc5ccccc5c4)cc4oc5ccccc5c34)n2)cc1]

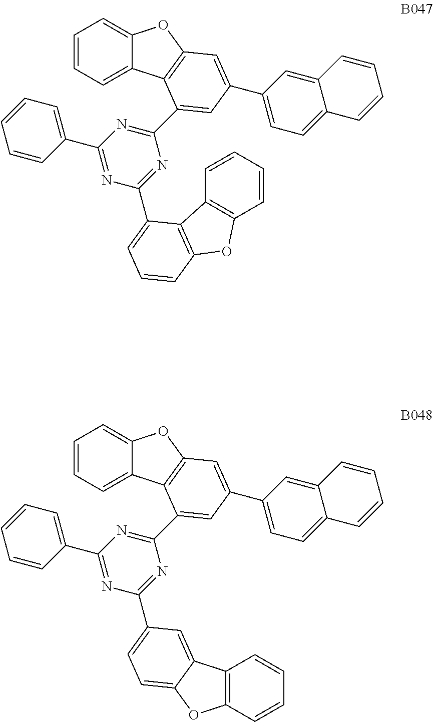 [SMILES: c1ccc(-c2nc(-c3cccc4oc5ccccc5c34)nc(-c3cc(-c4ccc5ccccc5c4)cc4oc5ccccc5c34)n2)cc1 ; c1ccc(-c2nc(-c3ccc4oc5ccccc5c4c3)nc(-c3cc(-c4ccc5ccccc5c4)cc4oc5ccccc5c34)n2)cc1]

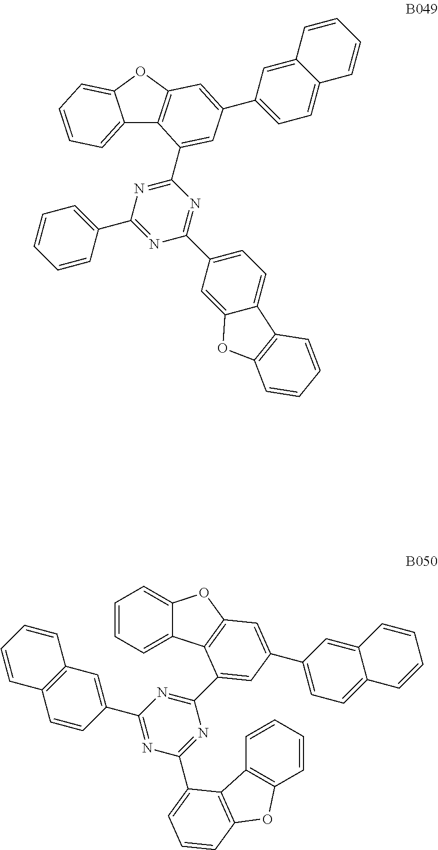 [SMILES: c1ccc(-c2nc(-c3ccc4c(c3)oc3ccccc34)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5cccc6oc7ccccc7c56)n4)c4c(c3)oc3ccccc34)ccc2c1]

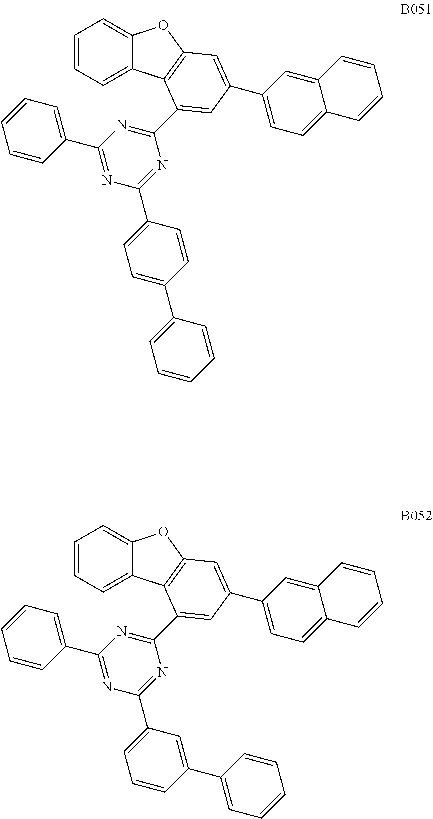 [SMILES: c1ccc(-c2ccc(-c3nc(-c4ccccc4)nc(-c4cc(-c5ccc6ccccc6c5)cc5oc6ccccc6c45)n3)cc2)cc1 ; c1ccc(-c2cccc(-c3nc(-c4ccccc4)nc(-c4cc(-c5ccc6ccccc6c5)cc5oc6ccccc6c45)n3)c2)cc1]

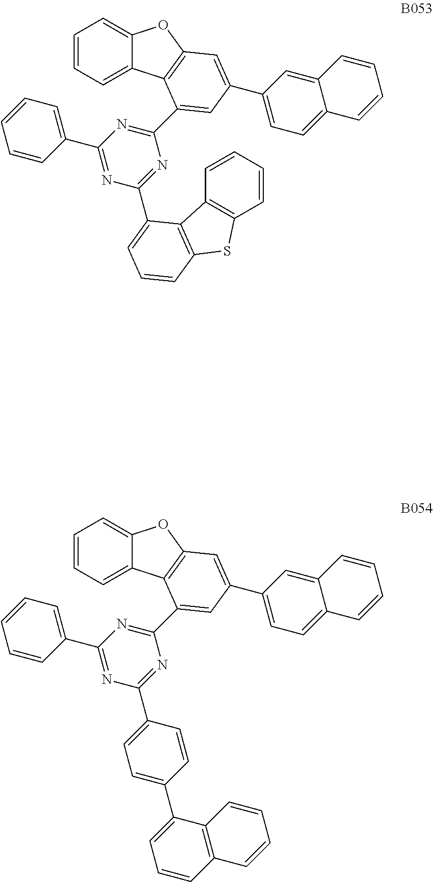 [SMILES: c1ccc(-c2nc(-c3cc(-c4ccc5ccccc5c4)cc4oc5ccccc5c34)nc(-c3cccc4sc5ccccc5c34)n2)cc1 ; c1ccc(-c2nc(-c3ccc(-c4cccc5ccccc45)cc3)nc(-c3cc(-c4ccc5ccccc5c4)cc4oc5ccccc5c34)n2)cc1]

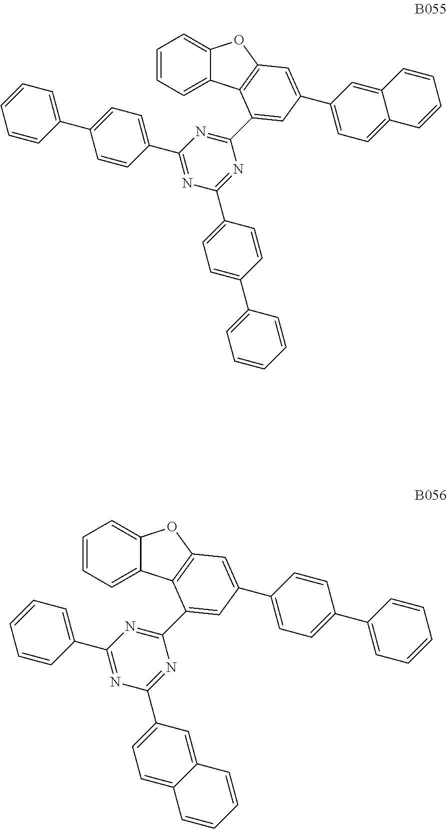 [SMILES: c1ccc(-c2ccc(-c3nc(-c4ccc(-c5ccccc5)cc4)nc(-c4cc(-c5ccc6ccccc6c5)cc5oc6ccccc6c45)n3)cc2)cc1 ; c1ccc(-c2ccc(-c3cc(-c4nc(-c5ccccc5)nc(-c5ccc6ccccc6c5)n4)c4c(c3)oc3ccccc34)cc2)cc1]

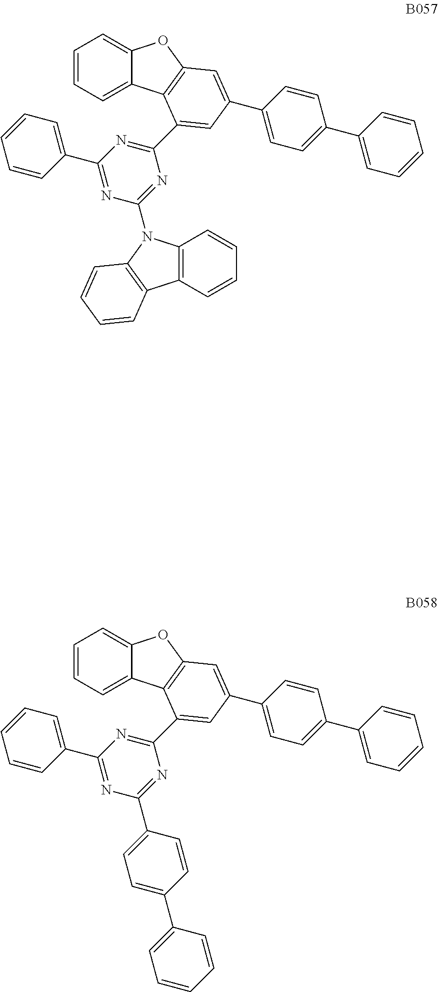 [SMILES: c1ccc(-c2ccc(-c3cc(-c4nc(-c5ccccc5)nc(-n5c6ccccc6c6ccccc65)n4)c4c(c3)oc3ccccc34)cc2)cc1 ; c1ccc(-c2ccc(-c3cc(-c4nc(-c5ccccc5)nc(-c5ccc(-c6ccccc6)cc5)n4)c4c(c3)oc3ccccc34)cc2)cc1]

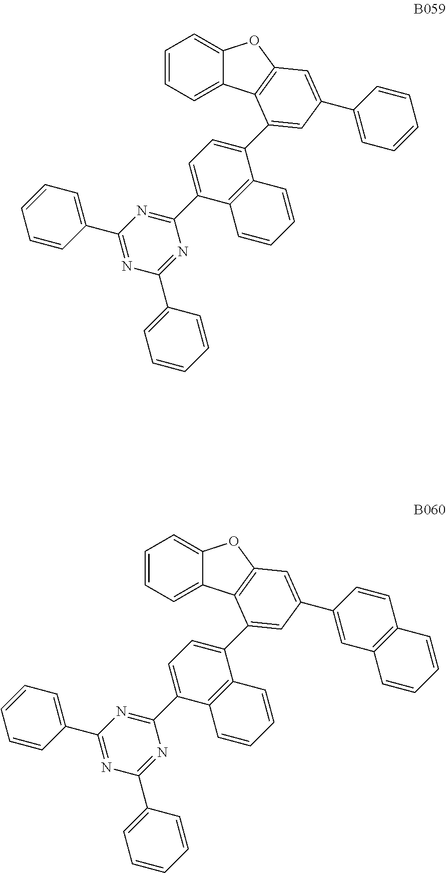 [SMILES: c1ccc(-c2cc(-c3ccc(-c4nc(-c5ccccc5)nc(-c5ccccc5)n4)c4ccccc34)c3c(c2)oc2ccccc23)cc1 ; c1ccc(-c2nc(-c3ccccc3)nc(-c3ccc(-c4cc(-c5ccc6ccccc6c5)cc5oc6ccccc6c45)c4ccccc34)n2)cc1]

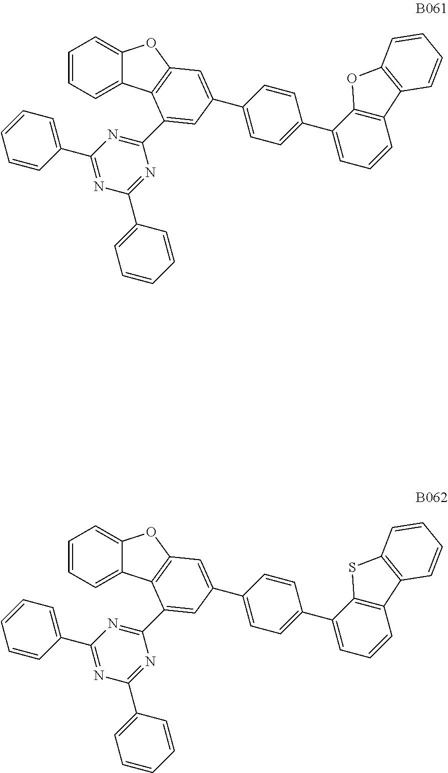 [SMILES: c1ccc(-c2nc(-c3ccccc3)nc(-c3cc(-c4ccc(-c5cccc6c5oc5ccccc56)cc4)cc4oc5ccccc5c34)n2)cc1 ; c1ccc(-c2nc(-c3ccccc3)nc(-c3cc(-c4ccc(-c5cccc6c5sc5ccccc56)cc4)cc4oc5ccccc5c34)n2)cc1]

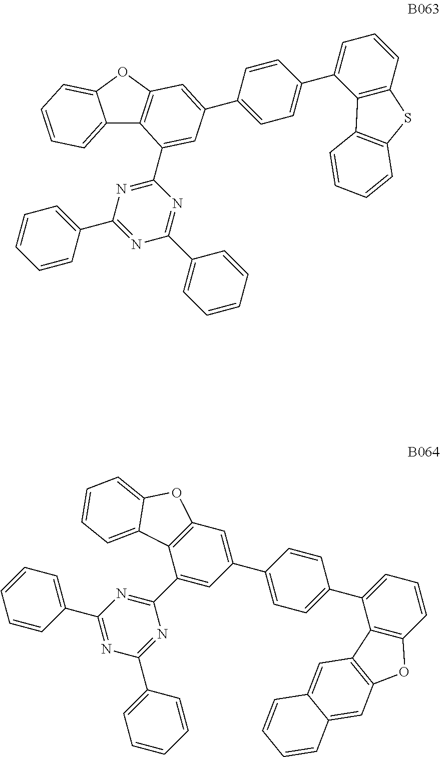 [SMILES: c1ccc(-c2nc(-c3ccccc3)nc(-c3cc(-c4ccc(-c5cccc6sc7ccccc7c56)cc4)cc4oc5ccccc5c34)n2)cc1 ; c1ccc(-c2nc(-c3ccccc3)nc(-c3cc(-c4ccc(-c5cccc6oc7cc8ccccc8cc7c56)cc4)cc4oc5ccccc5c34)n2)cc1]

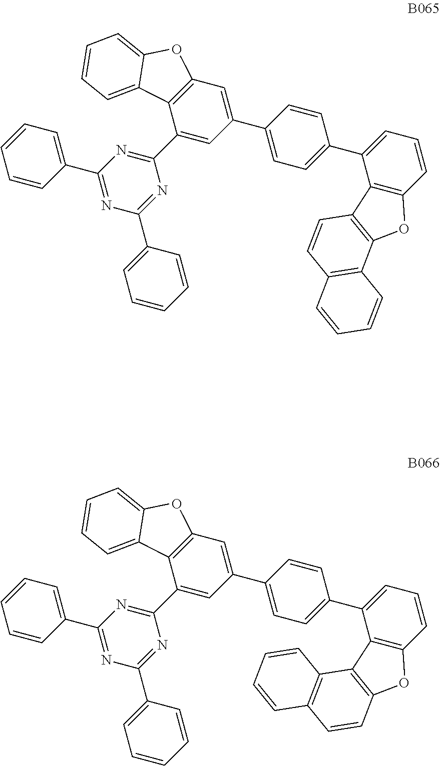 [SMILES: c1ccc(-c2nc(-c3ccccc3)nc(-c3cc(-c4ccc(-c5cccc6oc7c8ccccc8ccc7c56)cc4)cc4oc5ccccc5c34)n2)cc1 ; c1ccc(-c2nc(-c3ccccc3)nc(-c3cc(-c4ccc(-c5cccc6oc7ccc8ccccc8c7c56)cc4)cc4oc5ccccc5c34)n2)cc1]

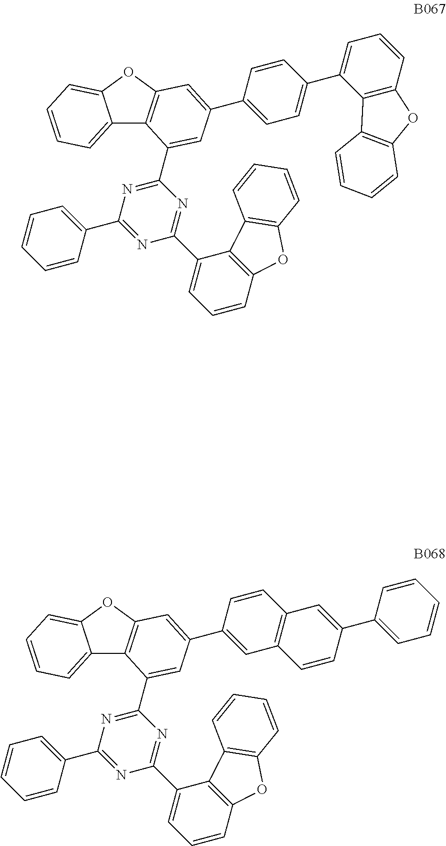 [SMILES: c1ccc(-c2nc(-c3cccc4oc5ccccc5c34)nc(-c3cc(-c4ccc(-c5cccc6oc7ccccc7c56)cc4)cc4oc5ccccc5c34)n2)cc1 ; c1ccc(-c2ccc3cc(-c4cc(-c5nc(-c6ccccc6)nc(-c6cccc7oc8ccccc8c67)n5)c5c(c4)oc4ccccc45)ccc3c2)cc1]

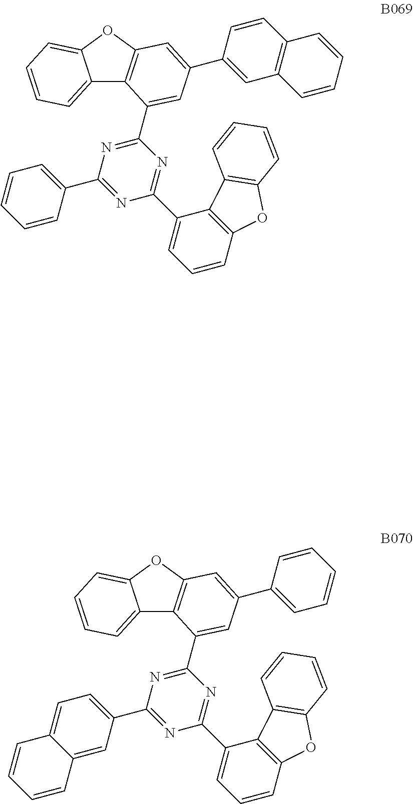 [SMILES: c1ccc(-c2nc(-c3cccc4oc5ccccc5c34)nc(-c3cc(-c4ccc5ccccc5c4)cc4oc5ccccc5c34)n2)cc1 ; c1ccc(-c2cc(-c3nc(-c4ccc5ccccc5c4)nc(-c4cccc5oc6ccccc6c45)n3)c3c(c2)oc2ccccc23)cc1]

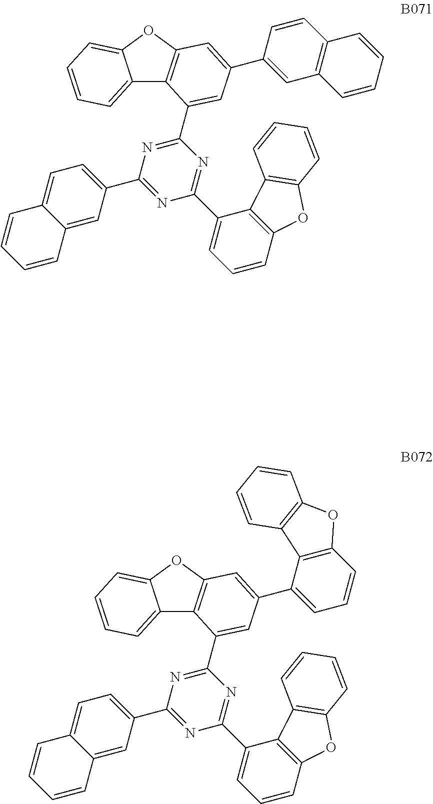 [SMILES: c1ccc2cc(-c3cc(-c4nc(-c5ccc6ccccc6c5)nc(-c5cccc6oc7ccccc7c56)n4)c4c(c3)oc3ccccc34)ccc2c1 ; c1ccc2cc(-c3nc(-c4cccc5oc6ccccc6c45)nc(-c4cc(-c5cccc6oc7ccccc7c56)cc5oc6ccccc6c45)n3)ccc2c1]

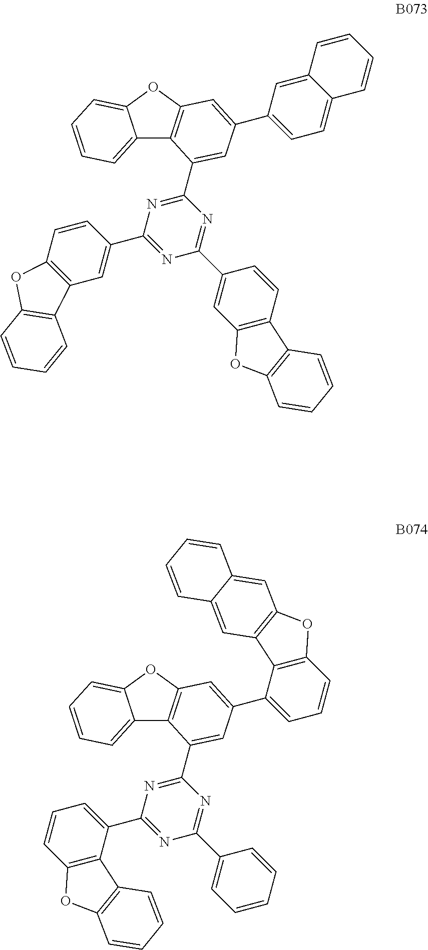 [SMILES: c1ccc2cc(-c3cc(-c4nc(-c5ccc6c(c5)oc5ccccc56)nc(-c5ccc6oc7ccccc7c6c5)n4)c4c(c3)oc3ccccc34)ccc2c1 ; c1ccc(-c2nc(-c3cccc4oc5ccccc5c34)nc(-c3cc(-c4cccc5oc6cc7ccccc7cc6c45)cc4oc5ccccc5c34)n2)cc1]

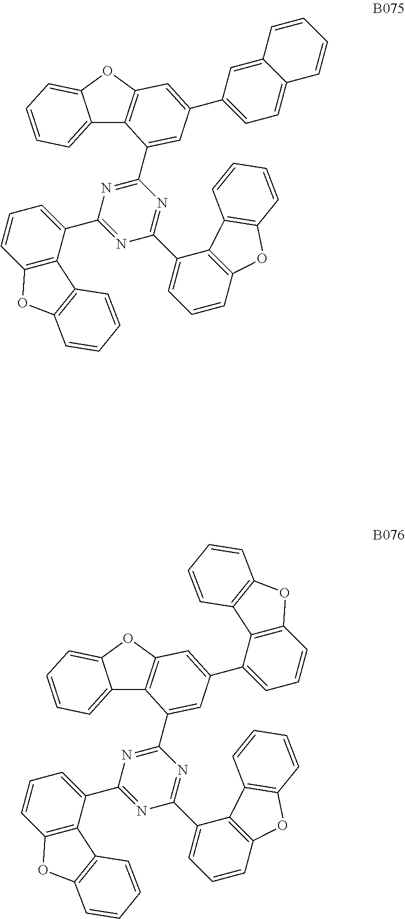 [SMILES: c1ccc2cc(-c3cc(-c4nc(-c5cccc6oc7ccccc7c56)nc(-c5cccc6oc7ccccc7c56)n4)c4c(c3)oc3ccccc34)ccc2c1 ; c1ccc2c(c1)oc1cccc(-c3cc(-c4nc(-c5cccc6oc7ccccc7c56)nc(-c5cccc6oc7ccccc7c56)n4)c4c(c3)oc3ccccc34)c12]

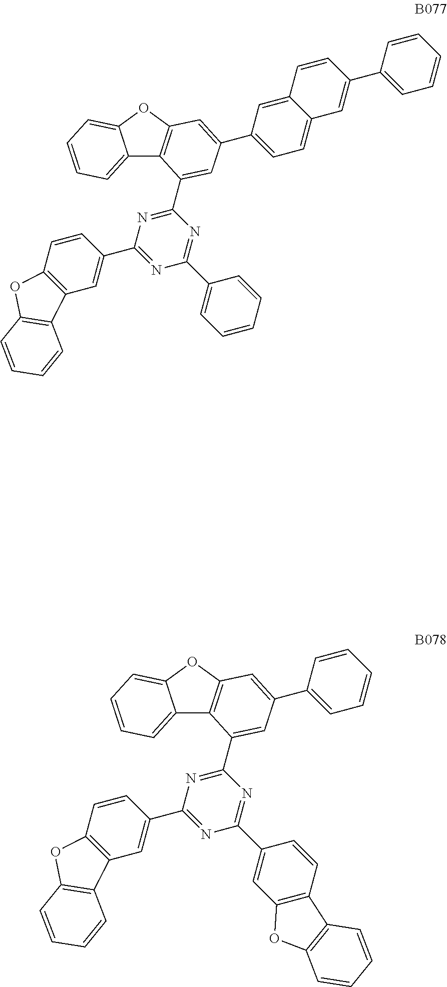 [SMILES: c1ccc(-c2ccc3cc(-c4cc(-c5nc(-c6ccccc6)nc(-c6ccc7oc8ccccc8c7c6)n5)c5c(c4)oc4ccccc45)ccc3c2)cc1 ; c1ccc(-c2cc(-c3nc(-c4ccc5c(c4)oc4ccccc45)nc(-c4ccc5oc6ccccc6c5c4)n3)c3c(c2)oc2ccccc23)cc1]

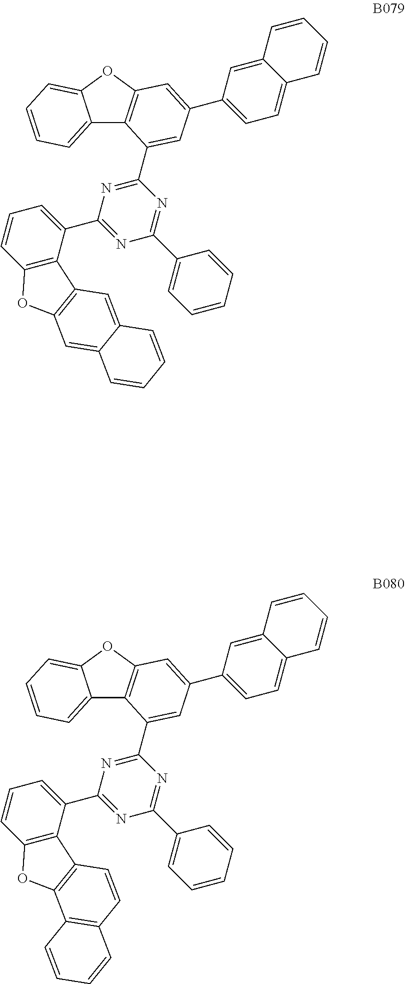 [SMILES: c1ccc(-c2nc(-c3cc(-c4ccc5ccccc5c4)cc4oc5ccccc5c34)nc(-c3cccc4oc5cc6ccccc6cc5c34)n2)cc1 ; c1ccc(-c2nc(-c3cc(-c4ccc5ccccc5c4)cc4oc5ccccc5c34)nc(-c3cccc4oc5c6ccccc6ccc5c34)n2)cc1]

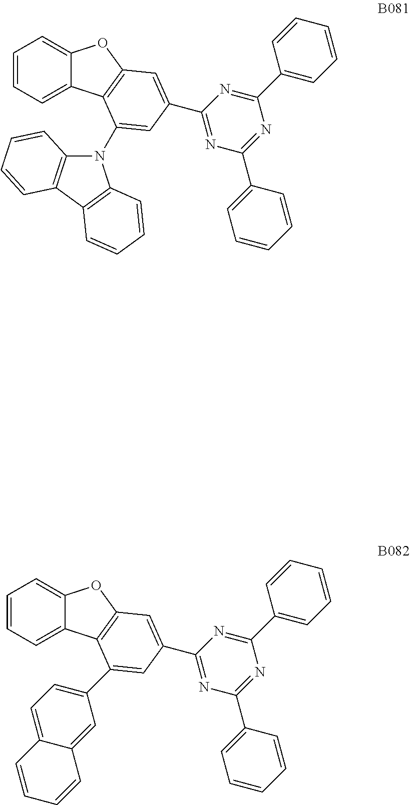 [SMILES: c1ccc(-c2nc(-c3ccccc3)nc(-c3cc(-n4c5ccccc5c5ccccc54)c4c(c3)oc3ccccc34)n2)cc1 ; c1ccc(-c2nc(-c3ccccc3)nc(-c3cc(-c4ccc5ccccc5c4)c4c(c3)oc3ccccc34)n2)cc1]

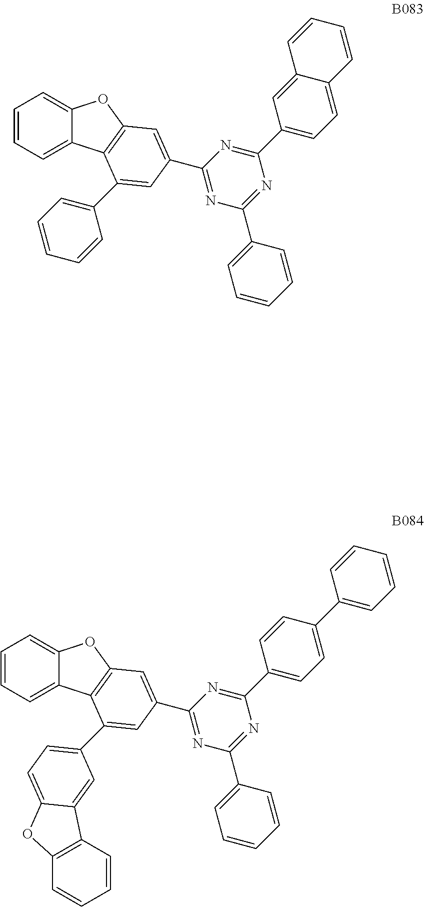 [SMILES: c1ccc(-c2nc(-c3ccc4ccccc4c3)nc(-c3cc(-c4ccccc4)c4c(c3)oc3ccccc34)n2)cc1 ; c1ccc(-c2ccc(-c3nc(-c4ccccc4)nc(-c4cc(-c5ccc6oc7ccccc7c6c5)c5c(c4)oc4ccccc45)n3)cc2)cc1]

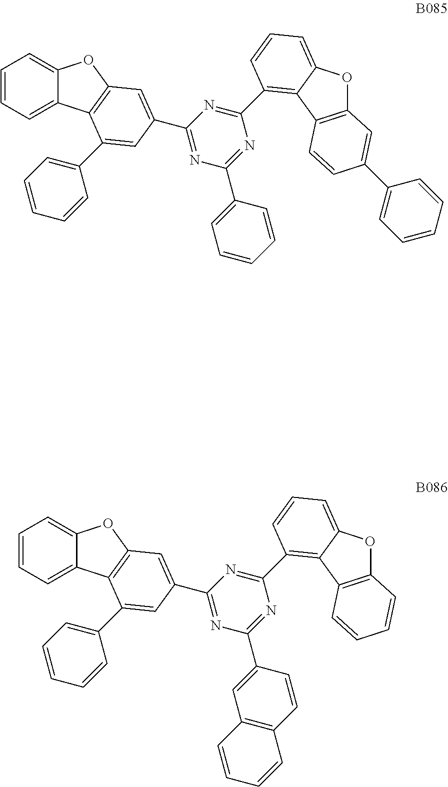 [SMILES: c1ccc(-c2ccc3c(c2)oc2cccc(-c4nc(-c5ccccc5)nc(-c5cc(-c6ccccc6)c6c(c5)oc5ccccc56)n4)c23)cc1 ; c1ccc(-c2cc(-c3nc(-c4ccc5ccccc5c4)nc(-c4cccc5oc6ccccc6c45)n3)cc3oc4ccccc4c23)cc1]

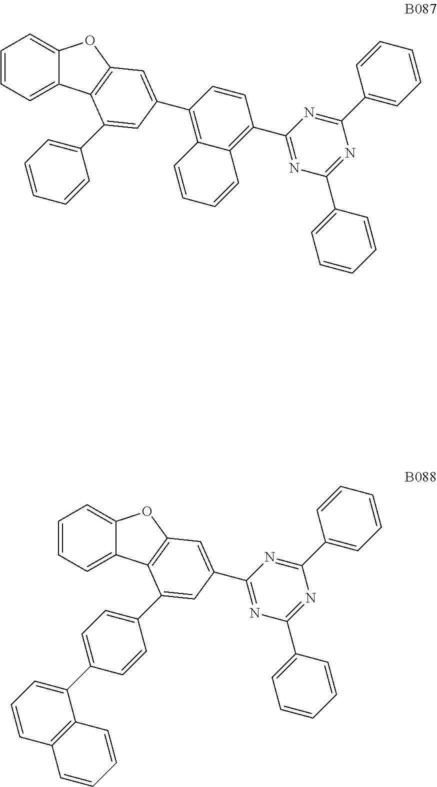 [SMILES: c1ccc(-c2nc(-c3ccccc3)nc(-c3ccc(-c4cc(-c5ccccc5)c5c(c4)oc4ccccc45)c4ccccc34)n2)cc1 ; c1ccc(-c2nc(-c3ccccc3)nc(-c3cc(-c4ccc(-c5cccc6ccccc56)cc4)c4c(c3)oc3ccccc34)n2)cc1]

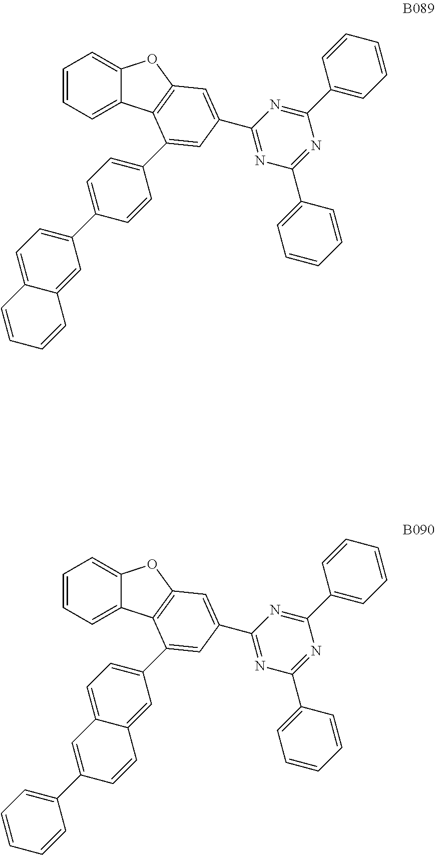 [SMILES: c1ccc(-c2nc(-c3ccccc3)nc(-c3cc(-c4ccc(-c5ccc6ccccc6c5)cc4)c4c(c3)oc3ccccc34)n2)cc1 ; c1ccc(-c2ccc3cc(-c4cc(-c5nc(-c6ccccc6)nc(-c6ccccc6)n5)cc5oc6ccccc6c45)ccc3c2)cc1]

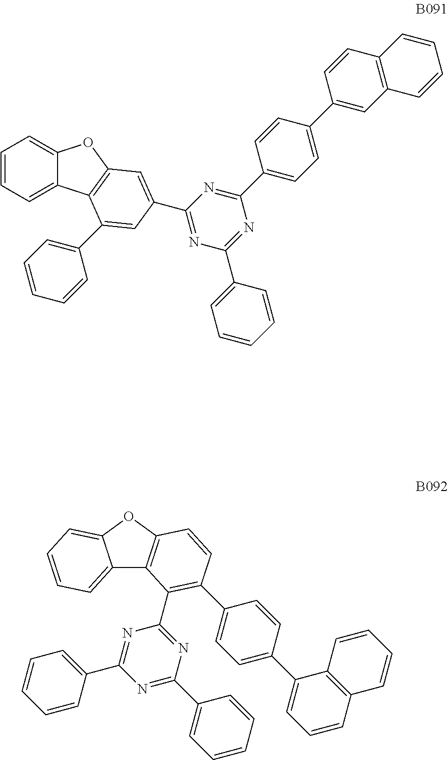 [SMILES: c1ccc(-c2nc(-c3ccc(-c4ccc5ccccc5c4)cc3)nc(-c3cc(-c4ccccc4)c4c(c3)oc3ccccc34)n2)cc1 ; c1ccc(-c2nc(-c3ccccc3)nc(-c3c(-c4ccc(-c5cccc6ccccc56)cc4)ccc4oc5ccccc5c34)n2)cc1]

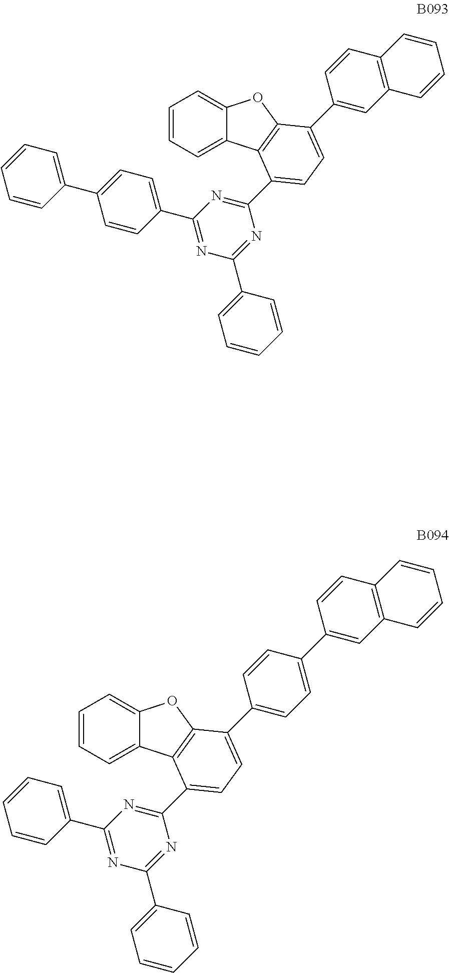 [SMILES: c1ccc(-c2ccc(-c3nc(-c4ccccc4)nc(-c4ccc(-c5ccc6ccccc6c5)c5oc6ccccc6c45)n3)cc2)cc1 ; c1ccc(-c2nc(-c3ccccc3)nc(-c3ccc(-c4ccc(-c5ccc6ccccc6c5)cc4)c4oc5ccccc5c34)n2)cc1]

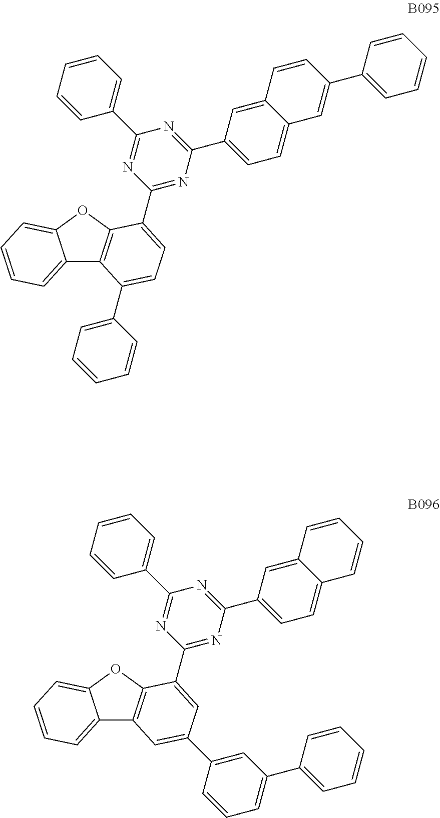 [SMILES: c1ccc(-c2ccc3cc(-c4nc(-c5ccccc5)nc(-c5ccc(-c6ccccc6)c6c5oc5ccccc56)n4)ccc3c2)cc1 ; c1ccc(-c2cccc(-c3cc(-c4nc(-c5ccccc5)nc(-c5ccc6ccccc6c5)n4)c4oc5ccccc5c4c3)c2)cc1]

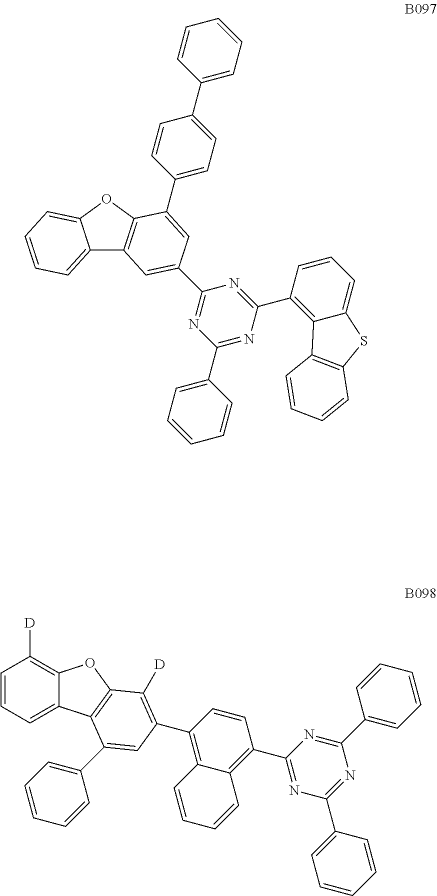 [SMILES: c1ccc(-c2ccc(-c3cc(-c4nc(-c5ccccc5)nc(-c5cccc6sc7ccccc7c56)n4)cc4c3oc3ccccc34)cc2)cc1 ; [2H]c1cccc2c1oc1c([2H])c(-c3ccc(-c4nc(-c5ccccc5)nc(-c5ccccc5)n4)c4ccccc34)cc(-c3ccccc3)c12]

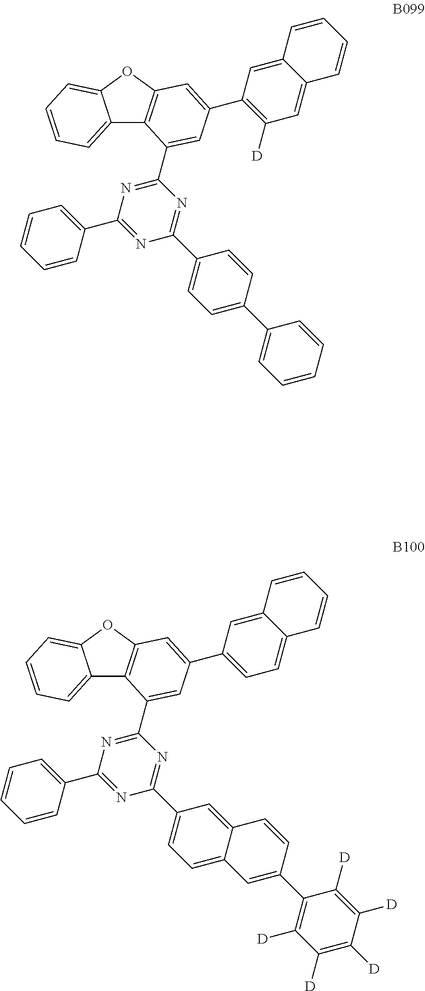 [SMILES: [2H]c1cc2ccccc2cc1-c1cc(-c2nc(-c3ccccc3)nc(-c3ccc(-c4ccccc4)cc3)n2)c2c(c1)oc1ccccc12 ; [2H]c1c([2H])c([2H])c(-c2ccc3cc(-c4nc(-c5ccccc5)nc(-c5cc(-c6ccc7ccccc7c6)cc6oc7ccccc7c56)n4)ccc3c2)c([2H])c1[2H]]

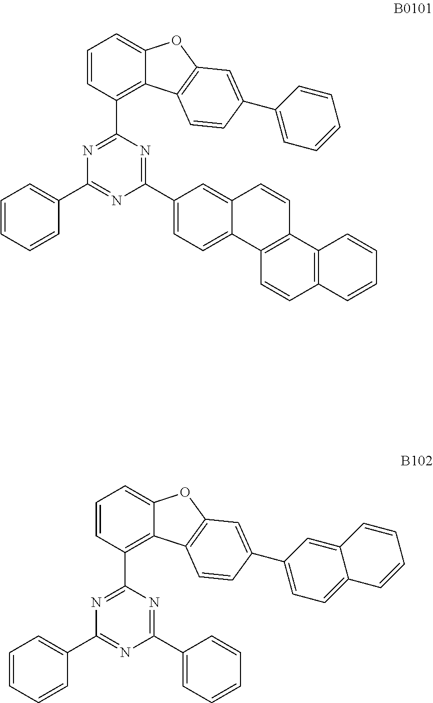 [SMILES: c1ccc(-c2ccc3c(c2)oc2cccc(-c4nc(-c5ccccc5)nc(-c5ccc6c(ccc7c8ccccc8ccc67)c5)n4)c23)cc1 ; c1ccc(-c2nc(-c3ccccc3)nc(-c3cccc4oc5cc(-c6ccc7ccccc7c6)ccc5c34)n2)cc1]

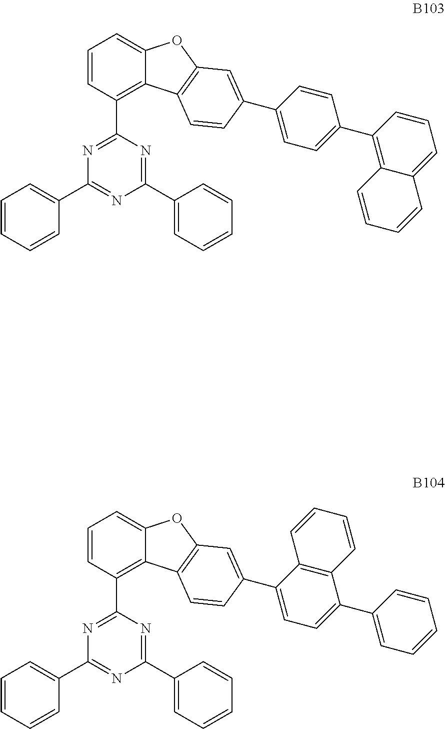 [SMILES: c1ccc(-c2nc(-c3ccccc3)nc(-c3cccc4oc5cc(-c6ccc(-c7cccc8ccccc78)cc6)ccc5c34)n2)cc1 ; c1ccc(-c2nc(-c3ccccc3)nc(-c3cccc4oc5cc(-c6ccc(-c7ccccc7)c7ccccc67)ccc5c34)n2)cc1]

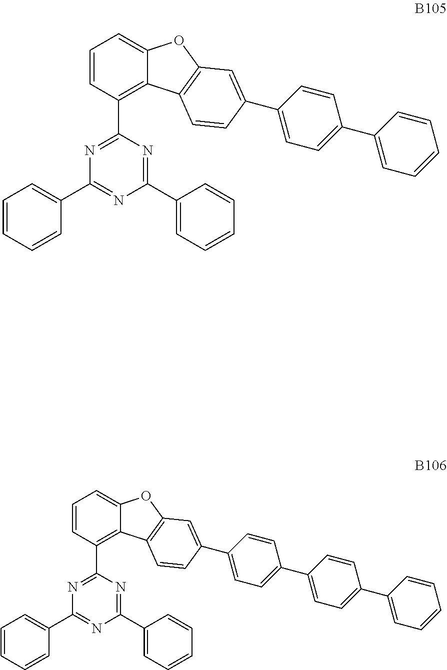 [SMILES: c1ccc(-c2ccc(-c3ccc4c(c3)oc3cccc(-c5nc(-c6ccccc6)nc(-c6ccccc6)n5)c34)cc2)cc1 ; c1ccc(-c2ccc(-c3ccc(-c4ccc5c(c4)oc4cccc(-c6nc(-c7ccccc7)nc(-c7ccccc7)n6)c45)cc3)cc2)cc1]

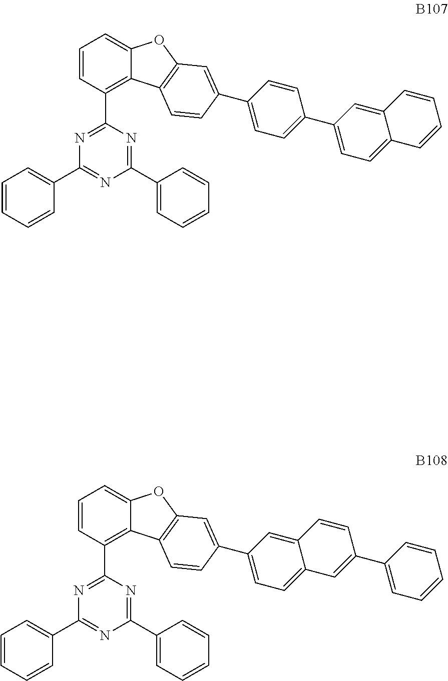 [SMILES: c1ccc(-c2nc(-c3ccccc3)nc(-c3cccc4oc5cc(-c6ccc(-c7ccc8ccccc8c7)cc6)ccc5c34)n2)cc1 ; c1ccc(-c2ccc3cc(-c4ccc5c(c4)oc4cccc(-c6nc(-c7ccccc7)nc(-c7ccccc7)n6)c45)ccc3c2)cc1]

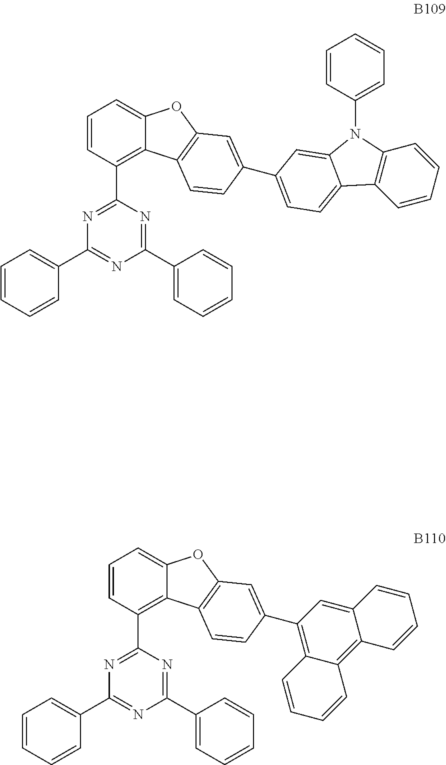 [SMILES: c1ccc(-c2nc(-c3ccccc3)nc(-c3cccc4oc5cc(-c6ccc7c8ccccc8n(-c8ccccc8)c7c6)ccc5c34)n2)cc1 ; c1ccc(-c2nc(-c3ccccc3)nc(-c3cccc4oc5cc(-c6cc7ccccc7c7ccccc67)ccc5c34)n2)cc1]

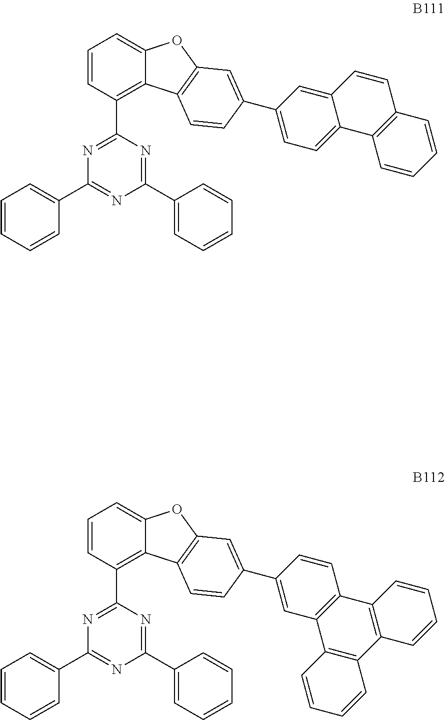 [SMILES: c1ccc(-c2nc(-c3ccccc3)nc(-c3cccc4oc5cc(-c6ccc7c(ccc8ccccc87)c6)ccc5c34)n2)cc1 ; c1ccc(-c2nc(-c3ccccc3)nc(-c3cccc4oc5cc(-c6ccc7c8ccccc8c8ccccc8c7c6)ccc5c34)n2)cc1]

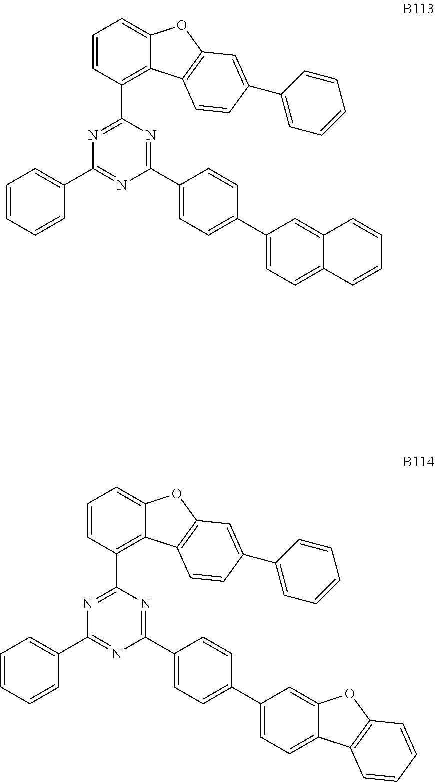 [SMILES: c1ccc(-c2ccc3c(c2)oc2cccc(-c4nc(-c5ccccc5)nc(-c5ccc(-c6ccc7ccccc7c6)cc5)n4)c23)cc1 ; c1ccc(-c2ccc3c(c2)oc2cccc(-c4nc(-c5ccccc5)nc(-c5ccc(-c6ccc7c(c6)oc6ccccc67)cc5)n4)c23)cc1]

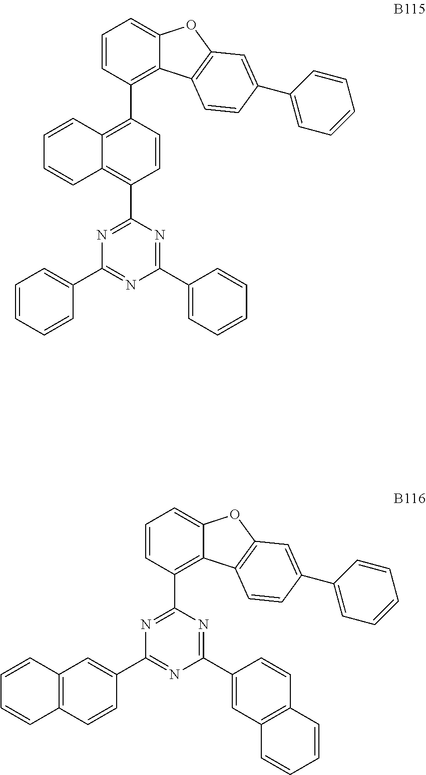 [SMILES: c1ccc(-c2ccc3c(c2)oc2cccc(-c4ccc(-c5nc(-c6ccccc6)nc(-c6ccccc6)n5)c5ccccc45)c23)cc1 ; c1ccc(-c2ccc3c(c2)oc2cccc(-c4nc(-c5ccc6ccccc6c5)nc(-c5ccc6ccccc6c5)n4)c23)cc1]

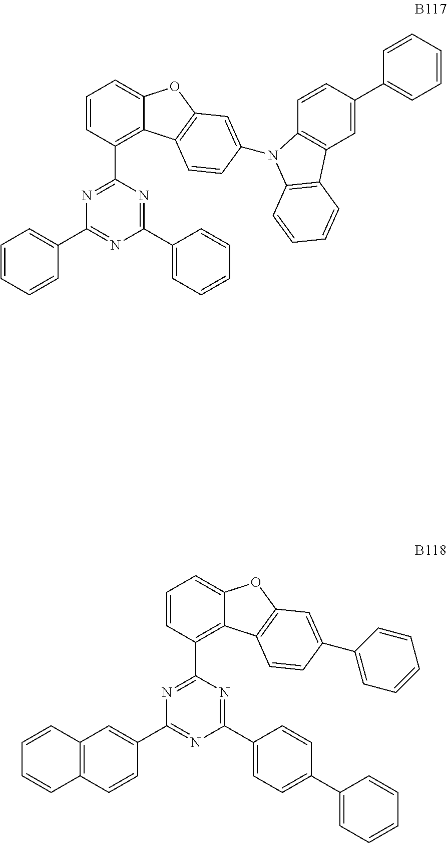 [SMILES: c1ccc(-c2ccc3c(c2)c2ccccc2n3-c2ccc3c(c2)oc2cccc(-c4nc(-c5ccccc5)nc(-c5ccccc5)n4)c23)cc1 ; c1ccc(-c2ccc(-c3nc(-c4ccc5ccccc5c4)nc(-c4cccc5oc6cc(-c7ccccc7)ccc6c45)n3)cc2)cc1]

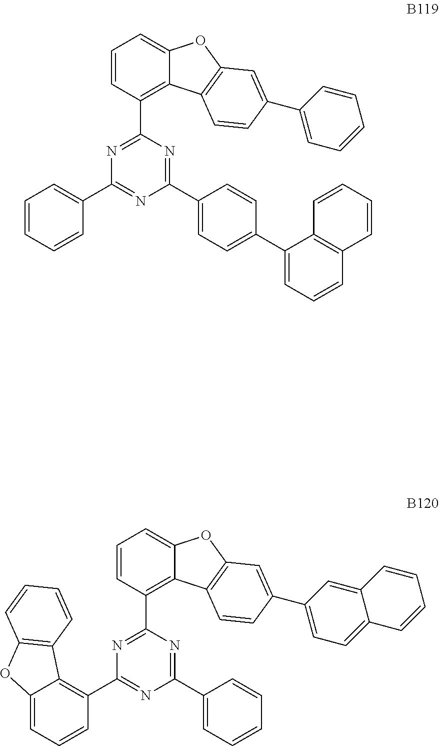 [SMILES: c1ccc(-c2ccc3c(c2)oc2cccc(-c4nc(-c5ccccc5)nc(-c5ccc(-c6cccc7ccccc67)cc5)n4)c23)cc1 ; c1ccc(-c2nc(-c3cccc4oc5ccccc5c34)nc(-c3cccc4oc5cc(-c6ccc7ccccc7c6)ccc5c34)n2)cc1]

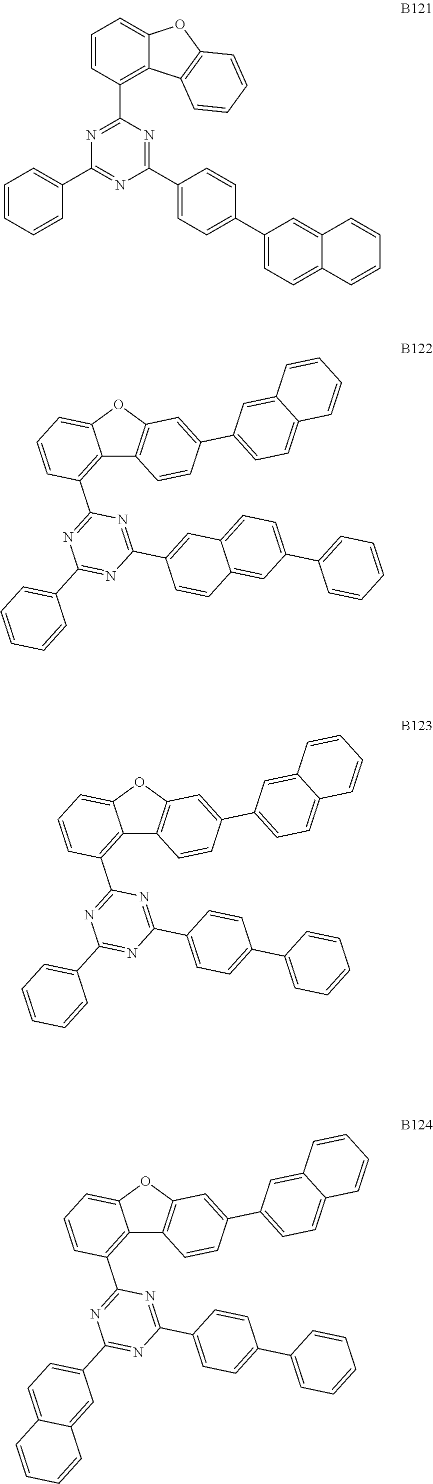 [SMILES: c1ccc(-c2nc(-c3ccc(-c4ccc5ccccc5c4)cc3)nc(-c3cccc4oc5ccccc5c34)n2)cc1 ; c1ccc(-c2ccc3cc(-c4nc(-c5ccccc5)nc(-c5cccc6oc7cc(-c8ccc9ccccc9c8)ccc7c56)n4)ccc3c2)cc1 ; c1ccc(-c2ccc(-c3nc(-c4ccccc4)nc(-c4cccc5oc6cc(-c7ccc8ccccc8c7)ccc6c45)n3)cc2)cc1 ; c1ccc(-c2ccc(-c3nc(-c4ccc5ccccc5c4)nc(-c4cccc5oc6cc(-c7ccc8ccccc8c7)ccc6c45)n3)cc2)cc1]

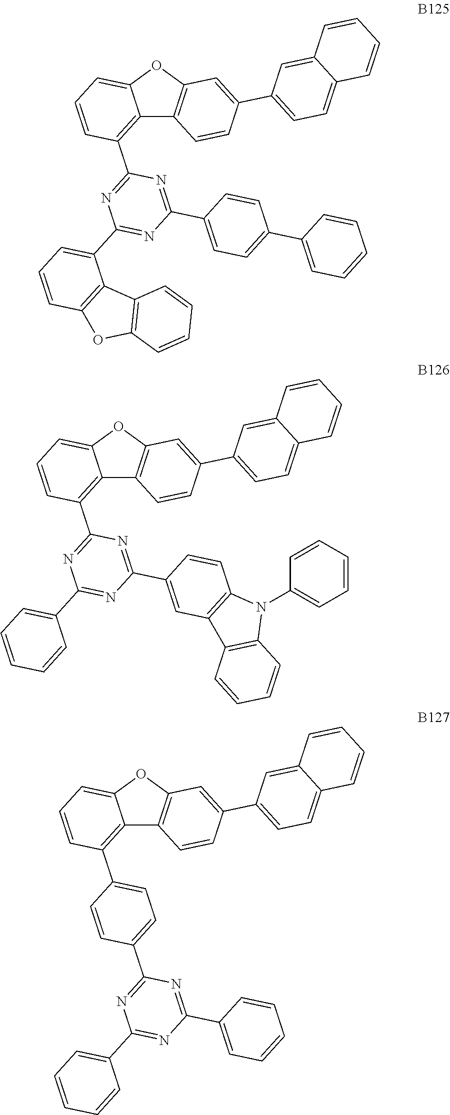 [SMILES: c1ccc(-c2ccc(-c3nc(-c4cccc5oc6ccccc6c45)nc(-c4cccc5oc6cc(-c7ccc8ccccc8c7)ccc6c45)n3)cc2)cc1 ; c1ccc(-c2nc(-c3ccc4c(c3)c3ccccc3n4-c3ccccc3)nc(-c3cccc4oc5cc(-c6ccc7ccccc7c6)ccc5c34)n2)cc1 ; c1ccc(-c2nc(-c3ccccc3)nc(-c3ccc(-c4cccc5oc6cc(-c7ccc8ccccc8c7)ccc6c45)cc3)n2)cc1]

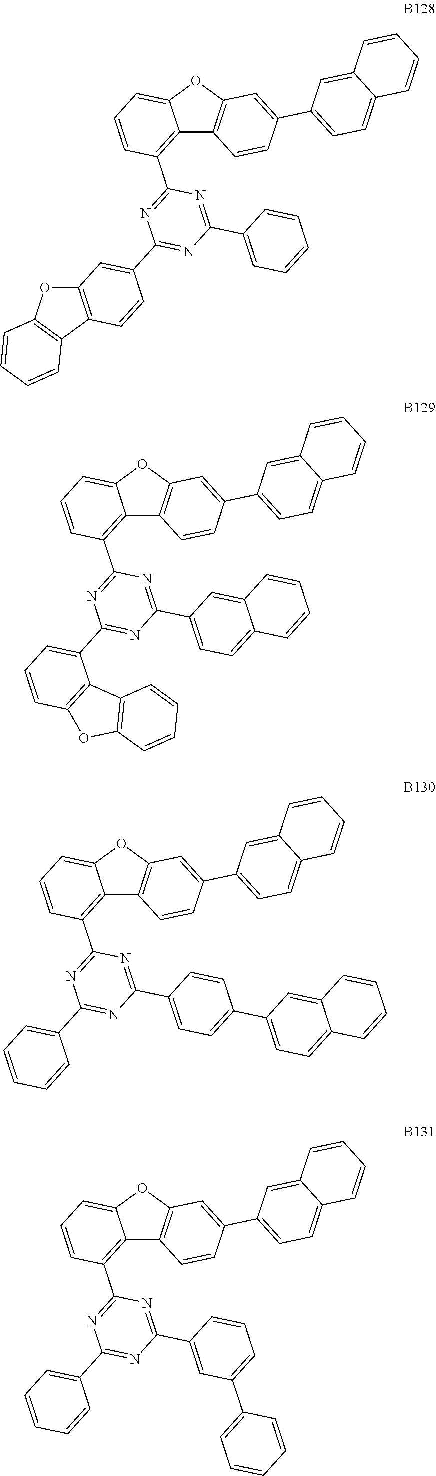 [SMILES: c1ccc(-c2nc(-c3ccc4c(c3)oc3ccccc34)nc(-c3cccc4oc5cc(-c6ccc7ccccc7c6)ccc5c34)n2)cc1 ; c1ccc2cc(-c3ccc4c(c3)oc3cccc(-c5nc(-c6ccc7ccccc7c6)nc(-c6cccc7oc8ccccc8c67)n5)c34)ccc2c1 ; c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2cccc(-c3nc(-c4ccccc4)nc(-c4cccc5oc6cc(-c7ccc8ccccc8c7)ccc6c45)n3)c2)cc1]

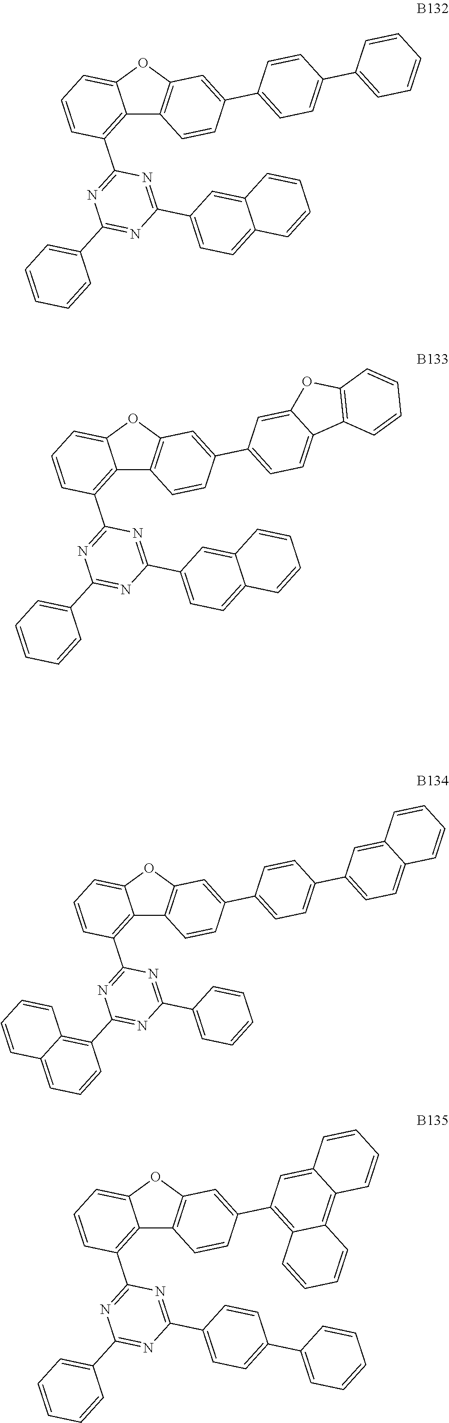 [SMILES: c1ccc(-c2ccc(-c3ccc4c(c3)oc3cccc(-c5nc(-c6ccccc6)nc(-c6ccc7ccccc7c6)n5)c34)cc2)cc1 ; c1ccc(-c2nc(-c3ccc4ccccc4c3)nc(-c3cccc4oc5cc(-c6ccc7c(c6)oc6ccccc67)ccc5c34)n2)cc1 ; c1ccc(-c2nc(-c3cccc4ccccc34)nc(-c3cccc4oc5cc(-c6ccc(-c7ccc8ccccc8c7)cc6)ccc5c34)n2)cc1 ; c1ccc(-c2ccc(-c3nc(-c4ccccc4)nc(-c4cccc5oc6cc(-c7cc8ccccc8c8ccccc78)ccc6c45)n3)cc2)cc1]

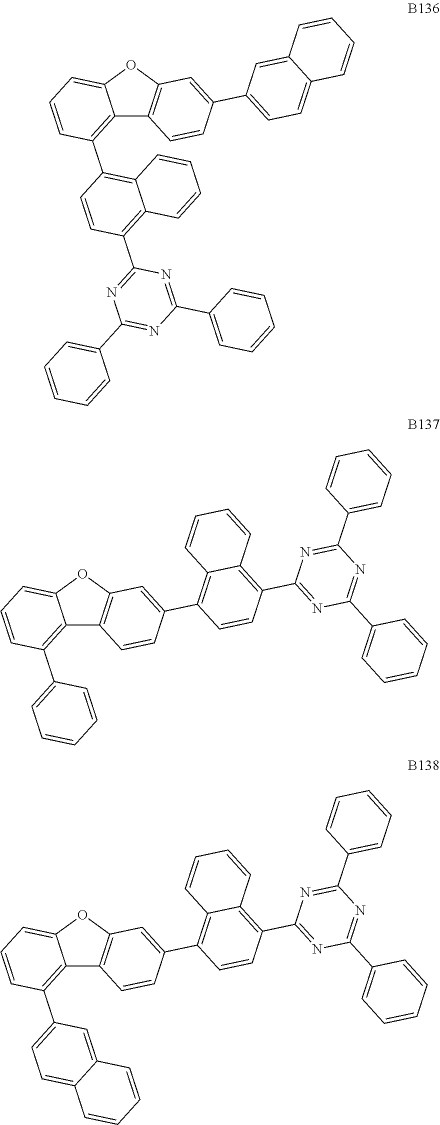 [SMILES: c1ccc(-c2nc(-c3ccccc3)nc(-c3ccc(-c4cccc5oc6cc(-c7ccc8ccccc8c7)ccc6c45)c4ccccc34)n2)cc1 ; c1ccc(-c2nc(-c3ccccc3)nc(-c3ccc(-c4ccc5c(c4)oc4cccc(-c6ccccc6)c45)c4ccccc34)n2)cc1 ; c1ccc(-c2nc(-c3ccccc3)nc(-c3ccc(-c4ccc5c(c4)oc4cccc(-c6ccc7ccccc7c6)c45)c4ccccc34)n2)cc1]

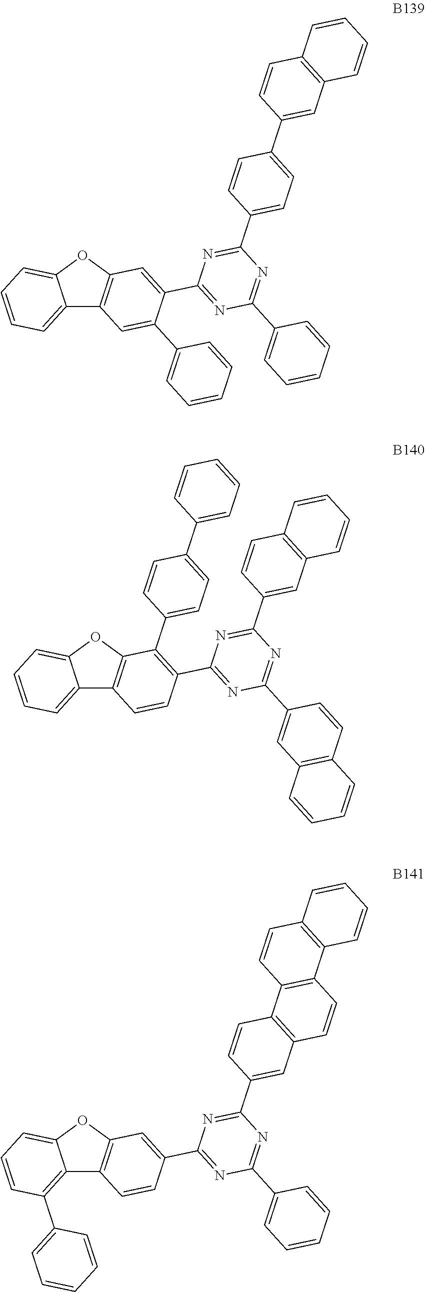 [SMILES: c1ccc(-c2nc(-c3ccc(-c4ccc5ccccc5c4)cc3)nc(-c3cc4oc5ccccc5c4cc3-c3ccccc3)n2)cc1 ; c1ccc(-c2ccc(-c3c(-c4nc(-c5ccc6ccccc6c5)nc(-c5ccc6ccccc6c5)n4)ccc4c3oc3ccccc34)cc2)cc1 ; c1ccc(-c2nc(-c3ccc4c(ccc5c6ccccc6ccc45)c3)nc(-c3ccc4c(c3)oc3cccc(-c5ccccc5)c34)n2)cc1]

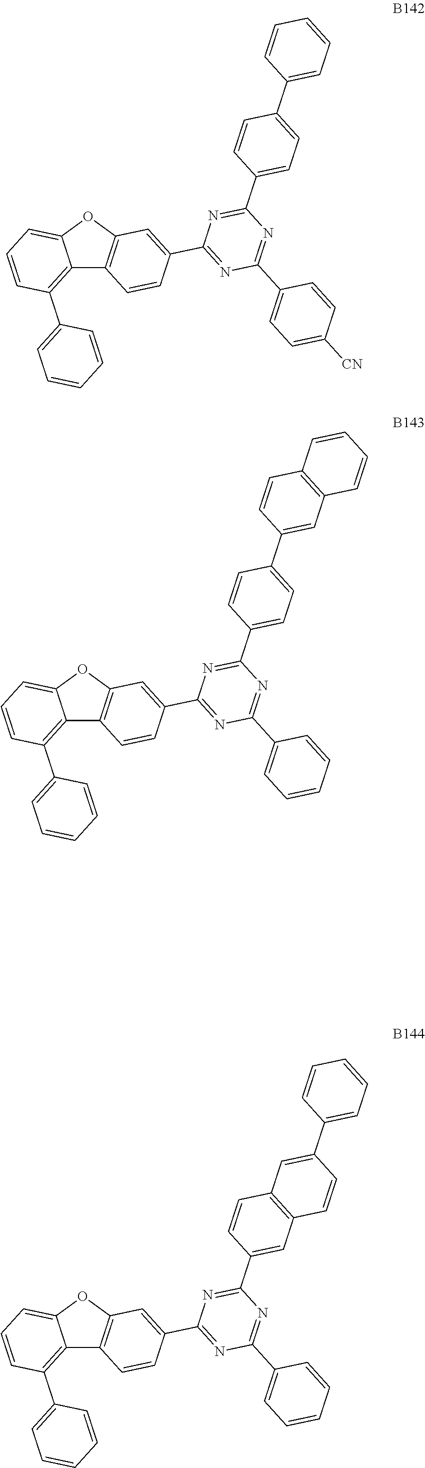 [SMILES: N#Cc1ccc(-c2nc(-c3ccc(-c4ccccc4)cc3)nc(-c3ccc4c(c3)oc3cccc(-c5ccccc5)c34)n2)cc1 ; c1ccc(-c2nc(-c3ccc(-c4ccc5ccccc5c4)cc3)nc(-c3ccc4c(c3)oc3cccc(-c5ccccc5)c34)n2)cc1 ; c1ccc(-c2ccc3cc(-c4nc(-c5ccccc5)nc(-c5ccc6c(c5)oc5cccc(-c7ccccc7)c56)n4)ccc3c2)cc1]

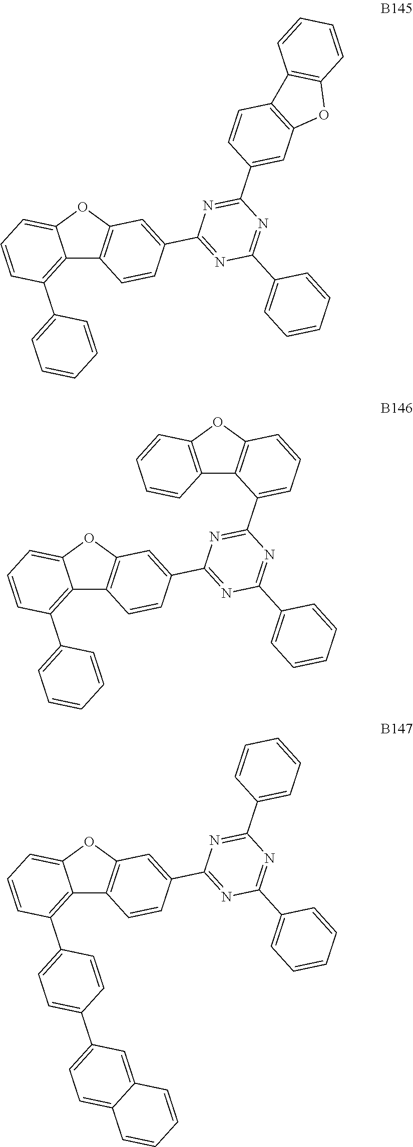 [SMILES: c1ccc(-c2nc(-c3ccc4c(c3)oc3ccccc34)nc(-c3ccc4c(c3)oc3cccc(-c5ccccc5)c34)n2)cc1 ; c1ccc(-c2nc(-c3ccc4c(c3)oc3cccc(-c5ccccc5)c34)nc(-c3cccc4oc5ccccc5c34)n2)cc1 ; c1ccc(-c2nc(-c3ccccc3)nc(-c3ccc4c(c3)oc3cccc(-c5ccc(-c6ccc7ccccc7c6)cc5)c34)n2)cc1]

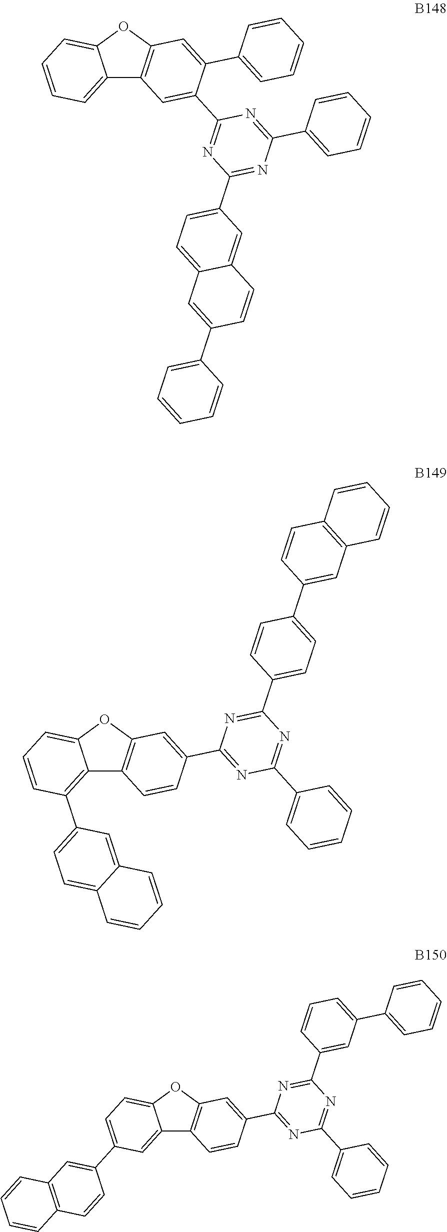 [SMILES: c1ccc(-c2ccc3cc(-c4nc(-c5ccccc5)nc(-c5cc6c(cc5-c5ccccc5)oc5ccccc56)n4)ccc3c2)cc1 ; c1ccc(-c2nc(-c3ccc(-c4ccc5ccccc5c4)cc3)nc(-c3ccc4c(c3)oc3cccc(-c5ccc6ccccc6c5)c34)n2)cc1 ; c1ccc(-c2cccc(-c3nc(-c4ccccc4)nc(-c4ccc5c(c4)oc4ccc(-c6ccc7ccccc7c6)cc45)n3)c2)cc1]

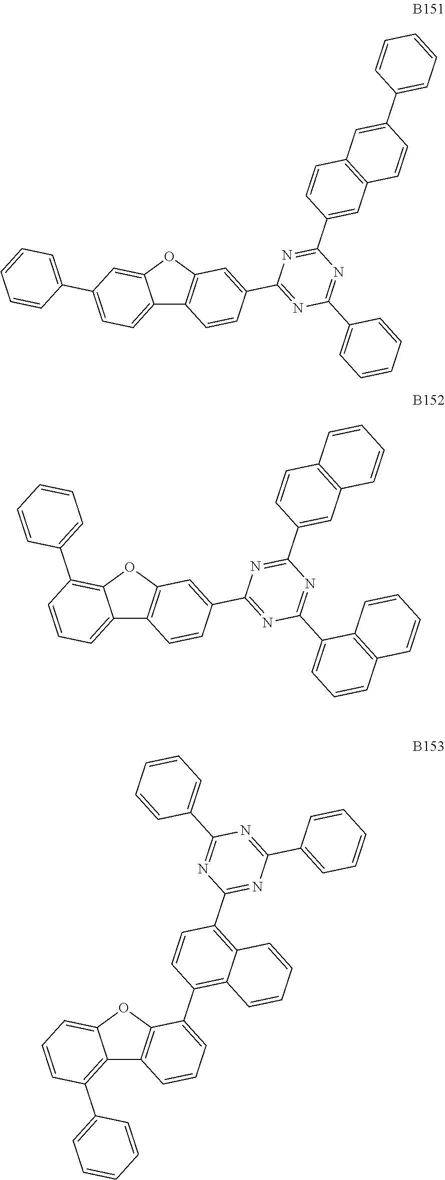 [SMILES: c1ccc(-c2ccc3cc(-c4nc(-c5ccccc5)nc(-c5ccc6c(c5)oc5cc(-c7ccccc7)ccc56)n4)ccc3c2)cc1 ; c1ccc(-c2cccc3c2oc2cc(-c4nc(-c5ccc6ccccc6c5)nc(-c5cccc6ccccc56)n4)ccc23)cc1 ; c1ccc(-c2nc(-c3ccccc3)nc(-c3ccc(-c4cccc5c4oc4cccc(-c6ccccc6)c45)c4ccccc34)n2)cc1]

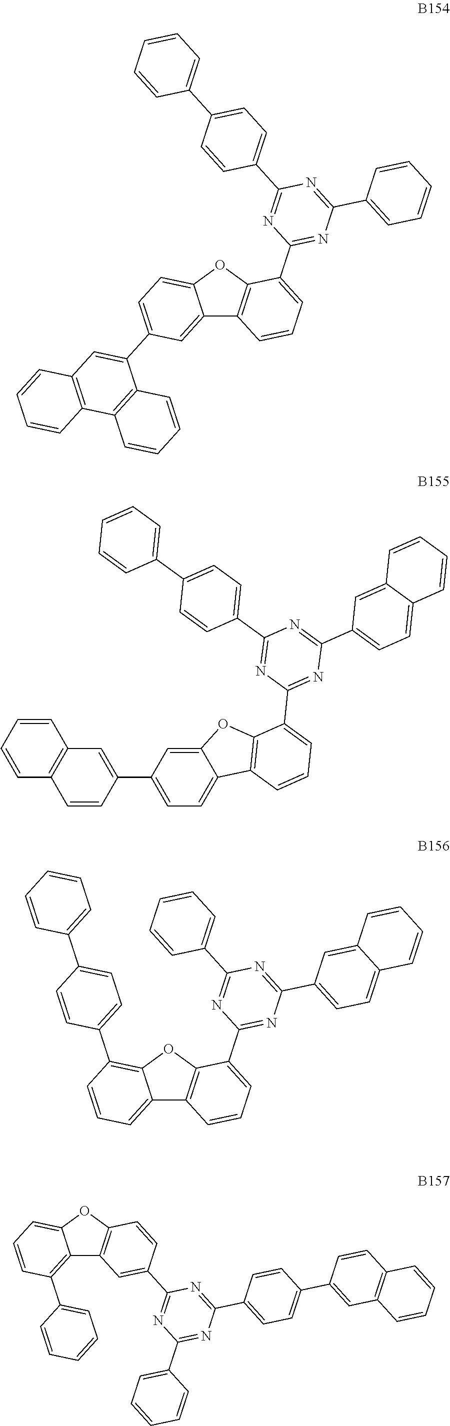 [SMILES: c1ccc(-c2ccc(-c3nc(-c4ccccc4)nc(-c4cccc5c4oc4ccc(-c6cc7ccccc7c7ccccc67)cc45)n3)cc2)cc1 ; c1ccc(-c2ccc(-c3nc(-c4ccc5ccccc5c4)nc(-c4cccc5c4oc4cc(-c6ccc7ccccc7c6)ccc45)n3)cc2)cc1 ; c1ccc(-c2ccc(-c3cccc4c3oc3c(-c5nc(-c6ccccc6)nc(-c6ccc7ccccc7c6)n5)cccc34)cc2)cc1 ; c1ccc(-c2nc(-c3ccc(-c4ccc5ccccc5c4)cc3)nc(-c3ccc4oc5cccc(-c6ccccc6)c5c4c3)n2)cc1]

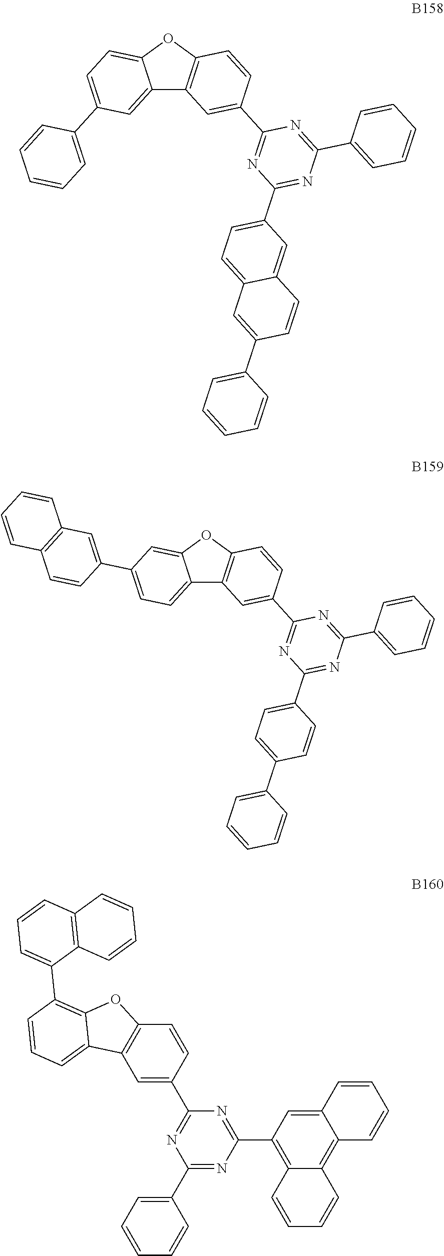 [SMILES: c1ccc(-c2ccc3cc(-c4nc(-c5ccccc5)nc(-c5ccc6oc7ccc(-c8ccccc8)cc7c6c5)n4)ccc3c2)cc1 ; c1ccc(-c2ccc(-c3nc(-c4ccccc4)nc(-c4ccc5oc6cc(-c7ccc8ccccc8c7)ccc6c5c4)n3)cc2)cc1 ; c1ccc(-c2nc(-c3ccc4oc5c(-c6cccc7ccccc67)cccc5c4c3)nc(-c3cc4ccccc4c4ccccc34)n2)cc1]

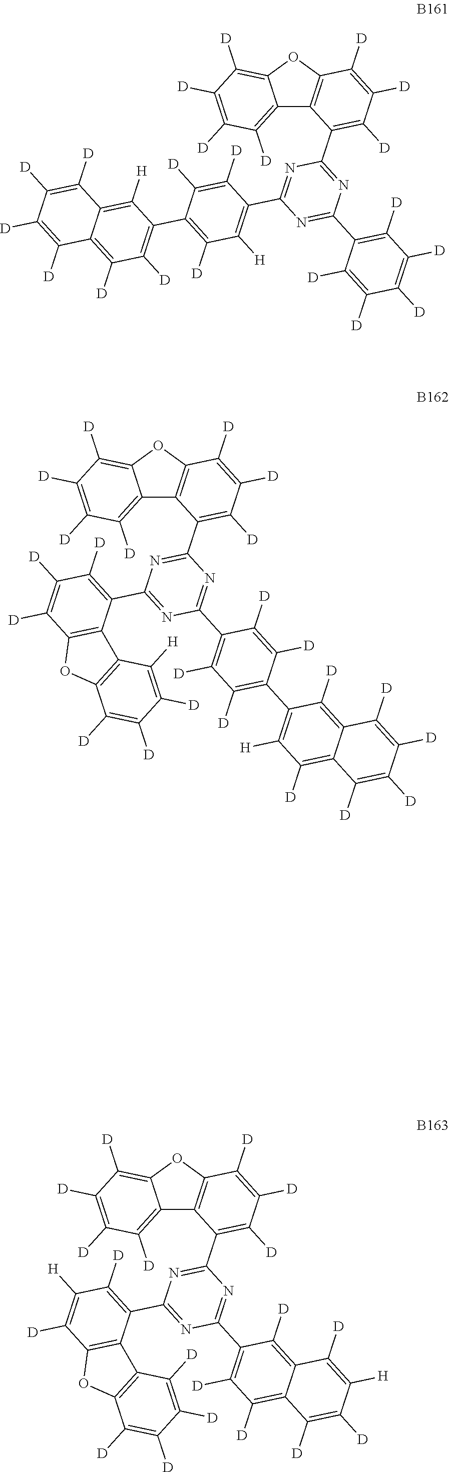

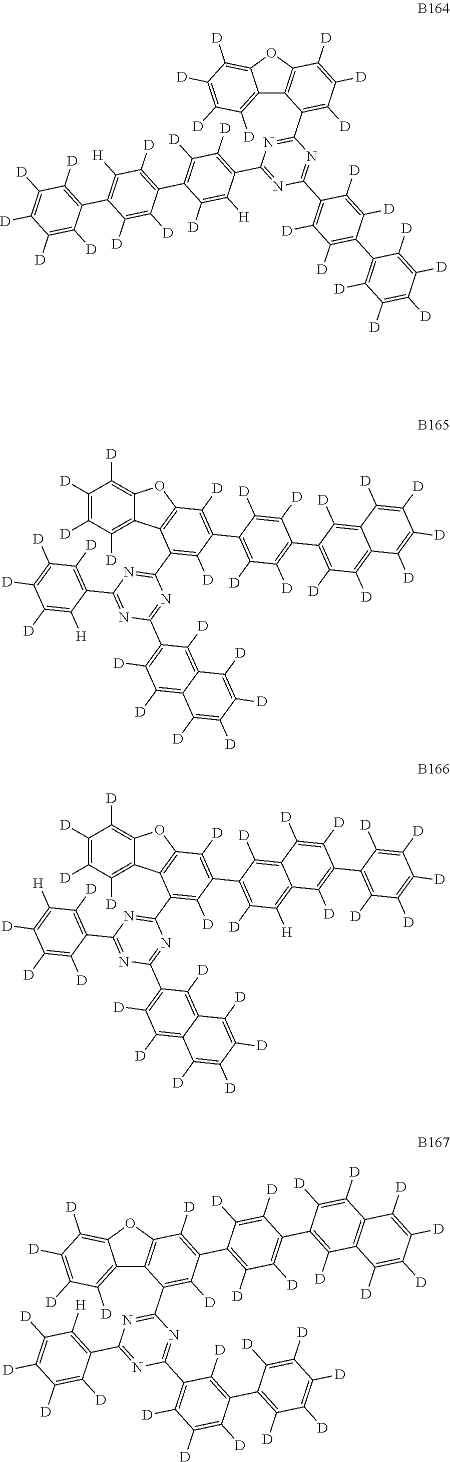

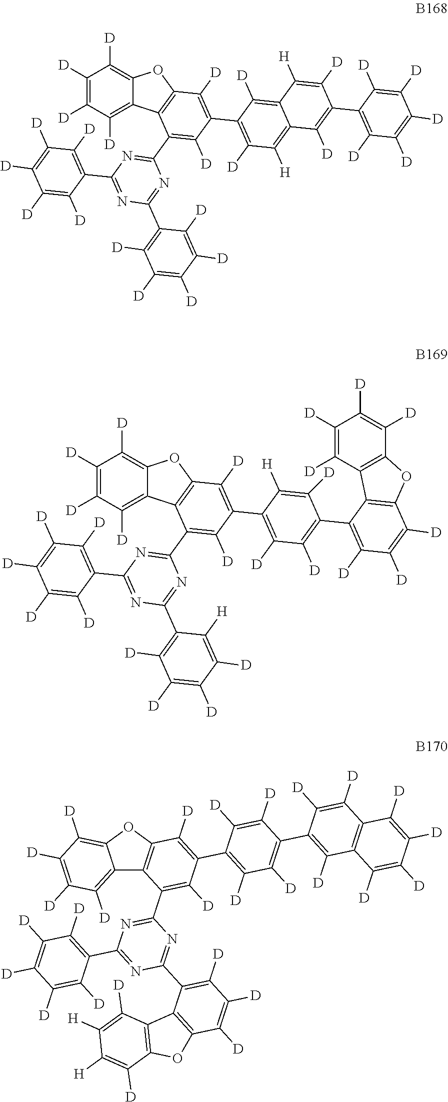

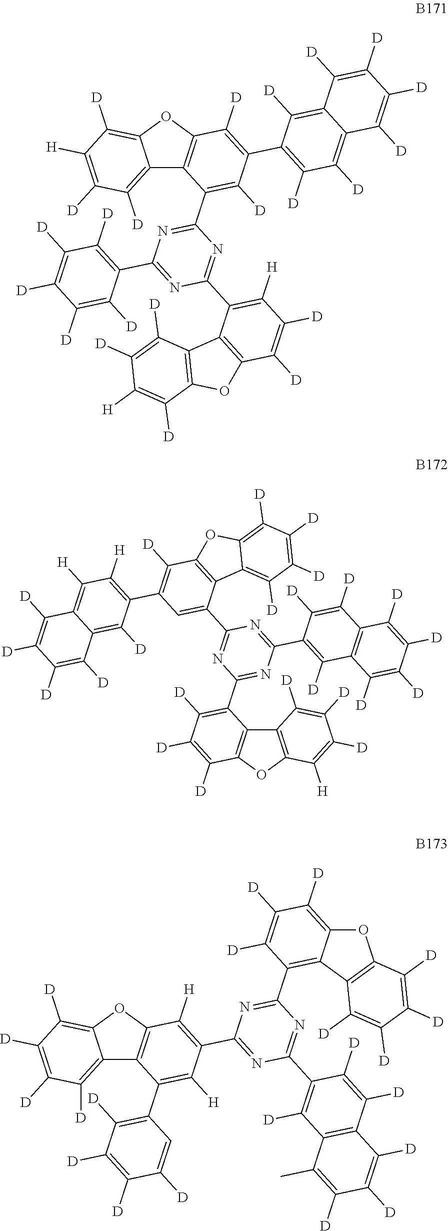

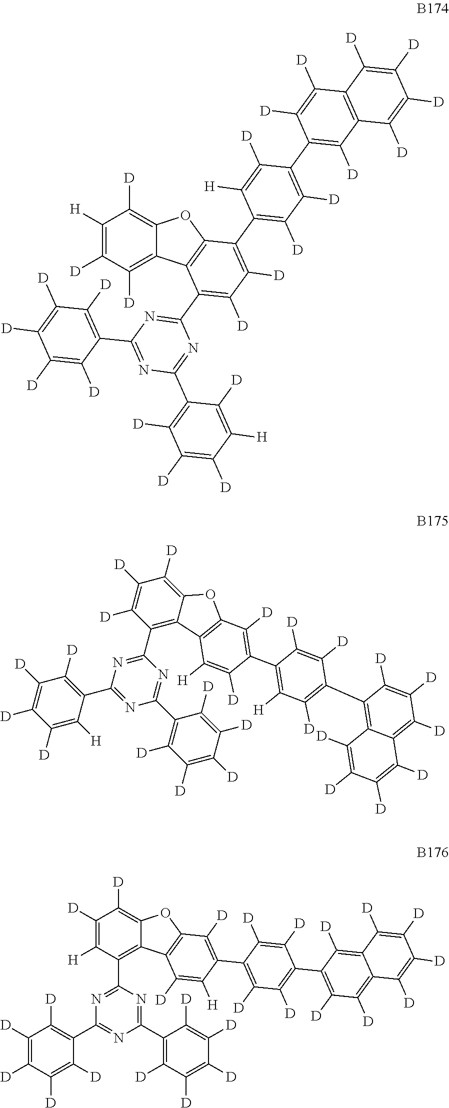

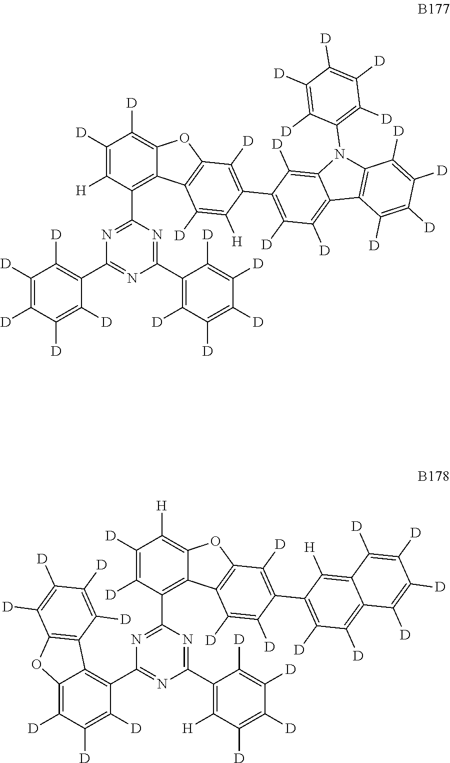 [SMILES: [2H]c1cc(-c2nc(-c3c([2H])c([2H])c([2H])c([2H])c3[2H])nc(-c3c([2H])c([2H])c([2H])c([2H])c3[2H])n2)c2c(oc3c([2H])c(-c4c([2H])c([2H])c5c6c([2H])c([2H])c([2H])c([2H])c6n(-c6c([2H])c([2H])c([2H])c([2H])c6[2H])c5c4[2H])cc([2H])c32)c1[2H] ; [2H]c1cc(-c2nc(-c3c([2H])c([2H])cc4oc5c([2H])c(-c6cc7c([2H])c([2H])c([2H])c([2H])c7c([2H])c6[2H])c([2H])c([2H])c5c34)nc(-c3c([2H])c([2H])c([2H])c4oc5c([2H])c([2H])c([2H])c([2H])c5c34)n2)c([2H])c([2H])c1[2H]]

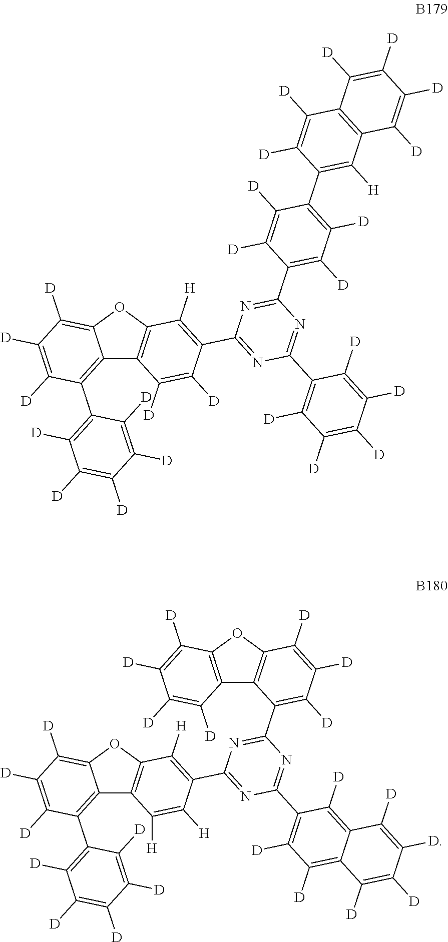

14. A composition for an organic layer of an organic light-emitting device, comprising:
the heterocyclic compound represented by Formula 1 according to claim 1; and
a compound represented by Formula 4 below: 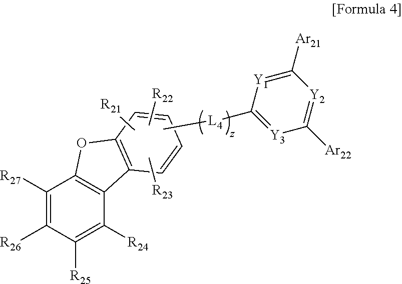
in Formula 4, any one of R₂₁ to R₂₇ is a substituted or unsubstituted aryl group having 6 to 60 carbon atoms, or a substituted or unsubstituted heteroaryl group having 2 to 60 carbon atoms,
the remaining groups of R₂₁ to R₂₇ are each independently selected from the group consisting of hydrogen, deuterium, a halogen, a cyano group, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted alkoxy group having 1 to 60 carbon atoms, a substituted or unsubstituted cycloalkyl group having 3 to 60 carbon atoms, a substituted or unsubstituted heterocycloalkyl group having 2 to 60 carbon atoms, a substituted or unsubstituted aryl group having 6 to 60 carbon atoms, a substituted or unsubstituted heteroaryl group having 2 to 60 carbon atoms, a phosphine oxide group, and a silyl group, or two or more adjacent groups are linked to form a substituted or unsubstituted aromatic hydrocarbon ring having 6 to 60 carbon atoms or a substituted or unsubstituted heterocycle having 2 to 60 carbon atoms,
L₄ is a direct linkage, a substituted or unsubstituted arylene group having 6 to 60 carbon atoms, or a substituted or unsubstituted heteroarylene group having 2 to 60 carbon atoms;
z is an integer of 0 to 5;
when z is an integer of 2 to 5, a plurality of L₄ groups are the same or different;
Y₁ to Y₃ are each independently CH, nitrogen (N), oxygen (O), or sulfur(S), and at least two of Y₁ to Y₃ are nitrogen; and
Ar₂₁ and Ar₂₂ are each independently a substituted or unsubstituted aryl group having 6 to 60 carbon atoms, or a substituted or unsubstituted heteroaryl group having 2 to 60 carbon atoms.

15. A composition for an organic layer of an organic light-emitting device according to claim 14, wherein the weight ratio of the compound represented by Formula 4 to the weight of the heterocyclic compound represented by Formula 1 in the total weight of the composition is 0.1 to 10.
